SEQUENCE LISTING (909 sequences)

SEQ ID NO: 1 - DNA, 4216 bp, synthetic construct
  misc_feature 1..4216: note = Synthetic
  misc_difference 2623: note = n is any nucleotide
  source 1..4216: mol_type = other DNA
gcatcacatcatcaccatcaccatcaccatggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccaggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccanggcacagccgaggccgctagcgccagcggcatgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcggcggcggctctggcggcggcggctccggcggcggcggctccagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgcc

SEQ ID NO: 2 - protein, 1406 aa, synthetic construct
  VAR_SEQ 875: note = Xaa is any amino acid
ASHHHHHHHHGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPXGTAEAASASGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 3 - DNA, 1587 bp, synthetic construct
  misc_feature 1..1587: note = Synthetic
  source 1..1587: mol_type = other DNA
atgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcggcggcggctctggcggcggcggctccggcggcggcggctccagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgcccatcatcaccatcaccatcaccat

SEQ ID NO: 4 - protein, 529 aa, synthetic construct
  REGION 1..529: note = Synthetic
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHHHH

SEQ ID NO: 5 - protein, 313 aa, synthetic construct
  REGION 1..313: note = Synthetic
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 6 - protein, 193 aa, synthetic construct
  REGION 1..193: note = Synthetic
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 7 - protein, 521 aa, synthetic construct
  REGION 1..521: note = Synthetic
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 8 - protein, 11 aa, synthetic construct
  REGION 1..11: note = Synthetic
YGRKKRRQRRR

SEQ ID NO: 9 - protein, 12 aa, synthetic construct
  REGION 1..12: note = Synthetic
RRQRRTSKLMKR

SEQ ID NO: 10 - protein, 27 aa, synthetic construct
  REGION 1..27: note = Synthetic
GWTLNSAGYLLGKINLKALAALAKKIL

SEQ ID NO: 11 - protein, 33 aa, synthetic construct
  REGION 1..33: note = Synthetic
KALAWEAKLAKALAKALAKHLAKALAKALKCEA

SEQ ID NO: 12 - protein, 16 aa, synthetic construct
  REGION 1..16: note = Synthetic
RQIKIWFQNRRMKWKK

SEQ ID NO: 13 - protein, 11 aa, synthetic construct
  REGION 1..11: note = Synthetic
YGRKKRRQRRR

SEQ ID NO: 14 - protein, 9 aa, synthetic construct
  REGION 1..9: note = Synthetic
RKKRRQRRR

SEQ ID NO: 15 - protein, 11 aa, synthetic construct
  REGION 1..11: note = Synthetic
YGRKKRRQRRR

SEQ ID NO: 16 - protein, 8 aa, synthetic construct
  REGION 1..8: note = Synthetic
RKKRRQRR

SEQ ID NO: 17 - protein, 11 aa, synthetic construct
  REGION 1..11: note = Synthetic
YARAAARQARA

SEQ ID NO: 18 - protein, 11 aa, synthetic construct
  REGION 1..11: note = Synthetic
THRLPRRRRRR

SEQ ID NO: 19 - protein, 11 aa, synthetic construct
  REGION 1..11: note = Synthetic
GGRRARRRRRR

SEQ ID NO: 20 - protein, 16 aa, synthetic construct
  REGION 1..16: note = Synthetic
LAGRSDNHSPLGLAGS

SEQ ID NO: 21 - protein, 16 aa, synthetic construct
  REGION 1..16: note = Synthetic
LAGRSDNHVPLSLSMG

SEQ ID NO: 22 - protein, 16 aa, synthetic construct
  REGION 1..16: note = Synthetic
LAGRSDNHEPLELVAG

SEQ ID NO: 23 - protein, 16 aa, synthetic construct
  REGION 1..16: note = Synthetic
ASGRSTNAGPSGLAGP

SEQ ID NO: 24 - protein, 16 aa, synthetic construct
  REGION 1..16: note = Synthetic
ASGRSTNAGPQGLAGQ

SEQ ID NO: 25 - protein, 16 aa, synthetic construct
  REGION 1..16: note = Synthetic
ASGRSTNAGPPGLTGP

SEQ ID NO: 26 - protein, 16 aa, synthetic construct
  REGION 1..16: note = Synthetic
ASSRGTNAGPAGLTGP

SEQ ID NO: 27 - protein, 16 aa, synthetic construct
  REGION 1..16: note = Synthetic
ASSRTTNTGPSTLTGP

SEQ ID NO: 28 - protein, 16 aa, synthetic construct
  REGION 1..16: note = Synthetic
AAGRSDNGTPLELVAP

SEQ ID NO: 29 - protein, 16 aa, synthetic construct
  REGION 1..16: note = Synthetic
EAGRSANHEPLGLVAT

SEQ ID NO: 30 - protein, 16 aa, synthetic construct
  REGION 1..16: note = Synthetic
ASGRGTNAGPAGLTGP

SEQ ID NO: 31 - protein, 16 aa, synthetic construct
  REGION 1..16: note = Synthetic
LFGRNDNHEPLELGGG

SEQ ID NO: 32 - protein, 16 aa, synthetic construct
  REGION 1..16: note = Synthetic
TAGRSDNLEPLGLVFG

SEQ ID NO: 33 - protein, 16 aa, synthetic construct
  REGION 1..16: note = Synthetic
LDGRSDNFHPPELVAG

SEQ ID NO: 34 - protein, 16 aa, synthetic construct
  REGION 1..16: note = Synthetic
LEGRSDNEEPENLVAG

SEQ ID NO: 35 - protein, 16 aa, synthetic construct
  REGION 1..16: note = Synthetic
LKGRSDNNAPLALVAG

SEQ ID NO: 36 - protein, 16 aa, synthetic construct
  REGION 1..16: note = Synthetic
VYSRGTNAGPHGLTGR

SEQ ID NO: 37 - protein, 16 aa, synthetic construct
  REGION 1..16: note = Synthetic
ANSRGTNKGFAGLIGP

SEQ ID NO: 38 - protein, 16 aa, synthetic construct
  REGION 1..16: note = Synthetic
ASSRLTNEAPAGLTIP

SEQ ID NO: 39 - protein, 16 aa, synthetic construct
  REGION 1..16: note = Synthetic
DQSRGTNAGPEGLTDP

SEQ ID NO: 40 - protein, 16 aa, synthetic construct
  REGION 1..16: note = Synthetic
ESSRGTNIGQGGLTGP

SEQ ID NO: 41 - protein, 16 aa, synthetic construct
  REGION 1..16: note = Synthetic
SSSRGTNQDPAGLTIP

SEQ ID NO: 42 - protein, 16 aa, synthetic construct
  REGION 1..16: note = Synthetic
ASSRGQNHSPMGLTGP

SEQ ID NO: 43 - protein, 16 aa, synthetic construct
  REGION 1..16: note = Synthetic
AYSRGPNAGPAGLEGR

SEQ ID NO: 44 - protein, 16 aa, synthetic construct
  REGION 1..16: note = Synthetic
ASERGNNAGPANLTGF

SEQ ID NO: 45 - protein, 16 aa, synthetic construct
  REGION 1..16: note = Synthetic
ASHRGTNPKPAILTGP

SEQ ID NO: 46 - protein, 16 aa, synthetic construct
  REGION 1..16: note = Synthetic
MSSRRTNANPAQLTGP

SEQ ID NO: 47 - protein, 16 aa, synthetic construct
  REGION 1..16: note = Synthetic
GAGRTDNHEPLELGAA

SEQ ID NO: 48 - protein, 16 aa, synthetic construct
  REGION 1..16: note = Synthetic
LAGRSENTAPLELTAG

SEQ ID NO: 49 - protein, 16 aa, synthetic construct
  REGION 1..16: note = Synthetic
LEGRPDNHEPLALVAS

SEQ ID NO: 50 - protein, 16 aa, synthetic construct
  REGION 1..16: note = Synthetic
LSGRSDNEEPLALPAG

SEQ ID NO: 51 - protein, 16 aa, synthetic construct
  REGION 1..16: note = Synthetic
EAGRTDNHEPLELSAP

SEQ ID NO: 52 - protein, 16 aa, synthetic construct
  REGION 1..16: note = Synthetic
EGGRSDNHGPLELVSG

SEQ ID NO: 53 - protein, 16 aa, synthetic construct
  REGION 1..16: note = Synthetic
LSGRSDNEAPLELEAG

SEQ ID NO: 54 - protein, 16 aa, synthetic construct
  REGION 1..16: note = Synthetic
LGGRADNHEPPELGAG

SEQ ID NO: 55 - protein, 16 aa, synthetic construct
  REGION 1..16: note = Synthetic
PPSRGTNAEPAGLTGE

SEQ ID NO: 56 - protein, 16 aa, synthetic construct
  REGION 1..16: note = Synthetic
ASTRGENAGPAGLEAP

SEQ ID NO: 57 - protein, 16 aa, synthetic construct
  REGION 1..16: note = Synthetic
ESSRGTNGAPEGLTGP

SEQ ID NO: 58 - protein, 16 aa, synthetic construct
  REGION 1..16: note = Synthetic
ASSRATNESPAGLTGE

SEQ ID NO: 59 - protein, 16 aa, synthetic construct
  REGION 1..16: note = Synthetic
ASSRGENPPPGGLTGP

SEQ ID NO: 60 - protein, 16 aa, synthetic construct
  REGION 1..16: note = Synthetic
AASRGTNTGPAELTGS

SEQ ID NO: 61 - protein, 16 aa, synthetic construct
  REGION 1..16: note = Synthetic
AGSRTTNAGPGGLEGP

SEQ ID NO: 62 - protein, 16 aa, synthetic construct
  REGION 1..16: note = Synthetic
APSRGENAGPATLTGA

SEQ ID NO: 63 - protein, 16 aa, synthetic construct
  REGION 1..16: note = Synthetic
ESGRAANTGPPTLTAP

SEQ ID NO: 64 - protein, 16 aa, synthetic construct
  REGION 1..16: note = Synthetic
NPGRAANEGPPGLPGS

SEQ ID NO: 65 - protein, 16 aa, synthetic construct
  REGION 1..16: note = Synthetic
ESSRAANLTPPELTGP

SEQ ID NO: 66 - protein, 16 aa, synthetic construct
  REGION 1..16: note = Synthetic
ASGRAANETPPGLTGA

SEQ ID NO: 67 - protein, 16 aa, synthetic construct
  REGION 1..16: note = Synthetic
NSGRGENLGAPGLTGT

SEQ ID NO: 68 - protein, 16 aa, synthetic construct
  REGION 1..16: note = Synthetic
TTGRAANLTPAGLTGP

SEQ ID NO: 69 - protein, 16 aa, synthetic construct
  REGION 1..16: note = Synthetic
EAGRSANHTPAGLTGP

SEQ ID NO: 70 - protein, 16 aa, synthetic construct
  REGION 1..16: note = Synthetic
ESGRAANTTPAGLTGP

SEQ ID NO: 71 - protein, 19 aa, synthetic construct
  REGION 1..19: note = Synthetic
TTGRATEAANLTPAGLTGP

SEQ ID NO: 72 - protein, 19 aa, synthetic construct
  REGION 1..19: note = Synthetic
TTGRAEEAANLTPAGLTGP

SEQ ID NO: 73 - protein, 19 aa, synthetic construct
  REGION 1..19: note = Synthetic
TTGRAGEAANLTPAGLTGP

SEQ ID NO: 74 - protein, 19 aa, synthetic construct
  REGION 1..19: note = Synthetic
TTGRATEAANATPAGLTGP

SEQ ID NO: 75 - protein, 19 aa, synthetic construct
  REGION 1..19: note = Synthetic
TTGRAGEAEGATSAGATGP

SEQ ID NO: 76 - protein, 19 aa, synthetic construct
  REGION 1..19: note = Synthetic
TTGEAGEAANATSAGATGP

SEQ ID NO: 77 - protein, 19 aa, synthetic construct
  REGION 1..19: note = Synthetic
TTGEAGEAAGLTPAGLTGP

SEQ ID NO: 78 - protein, 19 aa, synthetic construct
  REGION 1..19: note = Synthetic
TTGAAGEAANATPAGLTGP

SEQ ID NO: 79 - protein, 19 aa, synthetic construct
  REGION 1..19: note = Synthetic
TTGRAGEAAGLTPAGLTGP

SEQ ID NO: 80 - protein, 19 aa, synthetic construct
  REGION 1..19: note = Synthetic
TTGRAGEAANATSAGATGP

SEQ ID NO: 81 - protein, 19 aa, synthetic construct
  REGION 1..19: note = Synthetic
TTGEAGEAAGATSAGATGP

SEQ ID NO: 82 - protein, 16 aa, synthetic construct
  REGION 1..16: note = Synthetic
ESGRAANTEPPELGAG

SEQ ID NO: 83 - protein, 16 aa, synthetic construct
  REGION 1..16: note = Synthetic
ESGRAANTAPEGLTGP

SEQ ID NO: 84 - protein, 16 aa, synthetic construct
  REGION 1..16: note = Synthetic
EPGRAANHEPSGLTEG

SEQ ID NO: 85 - protein, 19 aa, synthetic construct
  REGION 1..19: note = Synthetic
ESGRAANHTGAPPGGLTGP

SEQ ID NO: 86 - protein, 19 aa, synthetic construct
  REGION 1..19: note = Synthetic
TTGRTGEGANATPGGLTGP

SEQ ID NO: 87 - protein, 19 aa, synthetic construct
  REGION 1..19: note = Synthetic
RTGRSGEAANETPEGLEGP

SEQ ID NO: 88 - protein, 19 aa, synthetic construct
  REGION 1..19: note = Synthetic
RTGRTGESANETPAGLGGP

SEQ ID NO: 89 - protein, 19 aa, synthetic construct
  REGION 1..19: note = Synthetic
STGRTGEPANETPAGLSGP

SEQ ID NO: 90 - protein, 19 aa, synthetic construct
  REGION 1..19: note = Synthetic
TTGRAGEPANATPTGLSGP

SEQ ID NO: 91 - protein, 19 aa, synthetic construct
  REGION 1..19: note = Synthetic
RTGRPGEGANATPTGLPGP

SEQ ID NO: 92 - protein, 19 aa, synthetic construct
  REGION 1..19: note = Synthetic
RTGRGGEAANATPSGLGGP

SEQ ID NO: 93 - protein, 19 aa, synthetic construct
  REGION 1..19: note = Synthetic
STGRSGESANATPGGLGGP

SEQ ID NO: 94 - protein, 19 aa, synthetic construct
  REGION 1..19: note = Synthetic
RTGRTGEEANATPAGLPGP

SEQ ID NO: 95 - protein, 19 aa, synthetic construct
  REGION 1..19: note = Synthetic
ATGRPGEPANTTPEGLEGP

SEQ ID NO: 96 - protein, 19 aa, synthetic construct
  REGION 1..19: note = Synthetic
STGRSGEPANATPGGLTGP

SEQ ID NO: 97 - protein, 19 aa, synthetic construct
  REGION 1..19: note = Synthetic
PTGRGGEGANTTPTGLPGP

SEQ ID NO: 98 - protein, 19 aa, synthetic construct
  REGION 1..19: note = Synthetic
PTGRSGEGANATPSGLTGP

SEQ ID NO: 99 - protein, 19 aa, synthetic construct
  REGION 1..19: note = Synthetic
TTGRASEGANSTPAPLTEP

SEQ ID NO: 100 - protein, 19 aa, synthetic construct
  REGION 1..19: note = Synthetic
TYGRAAEAANTTPAGLTAP

SEQ ID NO: 101 - protein, 19 aa, synthetic construct
  REGION 1..19: note = Synthetic
TTGRATEGANATPAELTEP

SEQ ID NO: 102 - protein, 19 aa, synthetic construct
  REGION 1..19: note = Synthetic
TVGRASEEANTTPASLTGP

SEQ ID NO: 103 - protein, 19 aa, synthetic construct
  REGION 1..19: note = Synthetic
TTGRAPEAANATPAPLTGP

SEQ ID NO: 104 - protein, 19 aa, synthetic construct
  REGION 1..19: note = Synthetic
TWGRATEPANATPAPLTSP

SEQ ID NO: 105 - protein, 19 aa, synthetic construct
  REGION 1..19: note = Synthetic
TVGRASESANATPAELTSP

SEQ ID NO: 106 - protein, 19 aa, synthetic construct
  REGION 1..19: note = Synthetic
TVGRAPEGANSTPAGLTGP

SEQ ID NO: 107 - protein, 19 aa, synthetic construct
  REGION 1..19: note = Synthetic
TWGRATEAPNLEPATLTTP

SEQ ID NO: 108 - protein, 19 aa, synthetic construct
  REGION 1..19: note = Synthetic
TTGRATEAPNLTPAPLTEP

SEQ ID NO: 109 - protein, 19 aa, synthetic construct
  REGION 1..19: note = Synthetic
TQGRATEAPNLSPAALTSP

SEQ ID NO: 110 - protein, 19 aa, synthetic construct
  REGION 1..19: note = Synthetic
TQGRAAEAPNLTPATLTAP

SEQ ID NO: 111 - protein, 19 aa, synthetic construct
  REGION 1..19: note = Synthetic
TSGRAPEATNLAPAPLTGP

SEQ ID NO: 112 - protein, 19 aa, synthetic construct
  REGION 1..19: note = Synthetic
TQGRAAEAANLTPAGLTEP

SEQ ID NO: 113 - protein, 19 aa, synthetic construct
  REGION 1..19: note = Synthetic
TTGRAGSAPNLPPTGLTTP

SEQ ID NO: 114 - protein, 19 aa, synthetic construct
  REGION 1..19: note = Synthetic
TTGRAGGAENLPPEGLTAP

SEQ ID NO: 115 - protein, 19 aa, synthetic construct
  REGION 1..19: note = Synthetic
TTSRAGTATNLTPEGLTAP

SEQ ID NO: 116 - protein, 19 aa, synthetic construct
  REGION 1..19: note = Synthetic
TTGRAGTATNLPPSGLTTP

SEQ ID NO: 117 - protein, 19 aa, synthetic construct
  REGION 1..19: note = Synthetic
TTARAGEAENLSPSGLTAP

SEQ ID NO: 118 - protein, 19 aa, synthetic construct
  REGION 1..19: note = Synthetic
TTGRAGGAGNLAPGGLTEP

SEQ ID NO: 119 - protein, 19 aa, synthetic construct
  REGION 1..19: note = Synthetic
TTGRAGTATNLPPEGLTGP

SEQ ID NO: 120 - protein, 19 aa, synthetic construct
  REGION 1..19: note = Synthetic
TTGRAGGAANLAPTGLTEP

SEQ ID NO: 121 - protein, 19 aa, synthetic construct
  REGION 1..19: note = Synthetic
TTGRAGTAENLAPSGLTTP

SEQ ID NO: 122 - protein, 19 aa, synthetic construct
  REGION 1..19: note = Synthetic
TTGRAGSATNLGPGGLTGP

SEQ ID NO: 123 - protein, 19 aa, synthetic construct
  REGION 1..19: note = Synthetic
TTARAGGAENLTPAGLTEP

SEQ ID NO: 124 - protein, 19 aa, synthetic construct
  REGION 1..19: note = Synthetic
TTARAGSAENLSPSGLTGP

SEQ ID NO: 125 - protein, 19 aa, synthetic construct
  REGION 1..19: note = Synthetic
TTARAGGAGNLAPEGLTTP

SEQ ID NO: 126 - protein, 19 aa, synthetic construct
  REGION 1..19: note = Synthetic
TTSRAGAAENLTPTGLTGP

SEQ ID NO: 127 - protein, 19 aa, synthetic construct
  REGION 1..19: note = Synthetic
TYGRTTTPGNEPPASLEAE

SEQ ID NO: 128 - protein, 19 aa, synthetic construct
  REGION 1..19: note = Synthetic
TYSRGESGPNEPPPGLTGP

SEQ ID NO: 129 - protein, 19 aa, synthetic construct
  REGION 1..19: note = Synthetic
AWGRTGASENETPAPLGGE

SEQ ID NO: 130 - protein, 19 aa, synthetic construct
  REGION 1..19: note = Synthetic
RWGRAETTPNTPPEGLETE

SEQ ID NO: 131 - protein, 19 aa, synthetic construct
  REGION 1..19: note = Synthetic
ESGRAANHTGAEPPELGAG

SEQ ID NO: 132 - protein, 19 aa, synthetic construct
  REGION 1..19: note = Synthetic
TTGRAGEAANLTPAGLTES

SEQ ID NO: 133 - protein, 19 aa, synthetic construct
  REGION 1..19: note = Synthetic
TTGRAGEAANLTPAALTES

SEQ ID NO: 134 - protein, 19 aa, synthetic construct
  REGION 1..19: note = Synthetic
TTGRAGEAANLTPAPLTES

SEQ ID NO: 135 - protein, 19 aa, synthetic construct
  REGION 1..19: note = Synthetic
TTGRAGEAANLTPEPLTES

SEQ ID NO: 136 - protein, 19 aa, synthetic construct
  REGION 1..19: note = Synthetic
TTGRAGEAANLTPAGLTGA

SEQ ID NO: 137 - protein, 19 aa, synthetic construct
  REGION 1..19: note = Synthetic
TTGRAGEAANLTPEGLTGA

SEQ ID NO: 138 - protein, 19 aa, synthetic construct
  REGION 1..19: note = Synthetic
TTGRAGEAANLTPEPLTGA

SEQ ID NO: 139 - protein, 19 aa, synthetic construct
  REGION 1..19: note = Synthetic
TTGRAGEAANLTPAGLTEA

SEQ ID NO: 140 - protein, 19 aa, synthetic construct
  REGION 1..19: note = Synthetic
TTGRAGEAANLTPEGLTEA

SEQ ID NO: 141 - protein, 19 aa, synthetic construct
  REGION 1..19: note = Synthetic
TTGRAGEAANLTPAPLTEA

SEQ ID NO: 142 - protein, 19 aa, synthetic construct
  REGION 1..19: note = Synthetic
TTGRAGEAANLTPEPLTEA

SEQ ID NO: 143 - protein, 19 aa, synthetic construct
  REGION 1..19: note = Synthetic
TTGRAGEAANLTPEPLTGP

SEQ ID NO: 144 - protein, 19 aa, synthetic construct
  REGION 1..19: note = Synthetic
TTGRAGEAANLTPAGLTGG

SEQ ID NO: 145 - protein, 19 aa, synthetic construct
  REGION 1..19: note = Synthetic
TTGRAGEAANLTPEGLTGG

SEQ ID NO: 146 - protein, 19 aa, synthetic construct
  REGION 1..19: note = Synthetic
TTGRAGEAANLTPEALTGG

SEQ ID NO: 147 - protein, 19 aa, synthetic construct
  REGION 1..19: note = Synthetic
TTGRAGEAANLTPEPLTGG

SEQ ID NO: 148 - protein, 19 aa, synthetic construct
  REGION 1..19: note = Synthetic
TTGRAGEAANLTPAGLTEG

SEQ ID NO: 149 - protein, 19 aa, synthetic construct
  REGION 1..19: note = Synthetic
TTGRAGEAANLTPEGLTEG

SEQ ID NO: 150 - protein, 19 aa, synthetic construct
  REGION 1..19: note = Synthetic
TTGRAGEAANLTPAPLTEG

SEQ ID NO: 151 - protein, 19 aa, synthetic construct
  REGION 1..19: note = Synthetic
TTGRAGEAANLTPEPLTEG

SEQ ID NO: 152 - protein, 177 aa, synthetic construct
  REGION 1..177: note = Synthetic
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 153 - protein, 175 aa, synthetic construct
  REGION 1..175: note = Synthetic
METCRCPLSYLISFLLFLPHSETACRLGKRPCRMQAFRIWDVNQKTFYLRNNQLVAGYLQGSNTKLEEKLDVVPVEPHAVFLGIHGGKLCLACVKSGDETRLQLEAVNITDLSKNKDQDKRFTFILSDSGPTTSFESAACPGWFLCTALEADRPVSLTNRPEEAMMVTKFYFQKE

SEQ ID NO: 154 - protein, 176 aa, synthetic construct
  REGION 1..176: note = Synthetic
MRPSRSTRRHLISLLLFLFHSETACRPSGKRPCRMQAFRIWDVNQKTFYLRNNQLVAGYLQGPNAKLEERIDVVPLEPQLLFLGIQRGKLCLSCVKSGDKMKLHLEAVNITDLGKNKEQDKRFTFIRSNSGPTTTFESASCPGWFLCTALEADQPVSLTNTPDDSIVVTKFYFQED

SEQ ID NO: 155 - protein, 178 aa, synthetic construct
  REGION 1..178: note = Synthetic
MEICRGPYSHLISLLLILLFRSESAGHIPAGKRPCKMQAFRIWDTNQKTFYLRNNQLIAGYLQGPNTKLEEKIDMVPIDFRNVFLGIHGGKLCLSCVKSGDDTKLQLEEVNITDLNKNKEEDKRFTFIRSETGPTTSFESLACPGWFLCTTLEADHPVSLTNTPKEPCTVTKFYFQED

SEQ ID NO: 156 - protein, 178 aa, synthetic construct
  REGION 1..178: note = Synthetic
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 157 - protein, 153 aa, synthetic construct
  REGION 1..153: note = Synthetic
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 158 - protein, 178 aa, synthetic construct
  REGION 1..178: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 159 - protein, 115 aa, synthetic construct
  REGION 1..115: note = Synthetic
EVQLLESGGGLVQPGGSLRLSCAASGFTFSSFSMSWVRQAPGKGLEWVSSISGSSGTTYYADSVGRFTISRDNSKNTLYLQMNSLRAEDTAVYYCAKPFPYFDYWGQGTLVTVSS

SEQ ID NO: 160 - protein, 108 aa, synthetic construct [sequence omitted]

SEQ ID NO: 161 - protein, 5 aa, synthetic construct
  REGION 1..5: note = Synthetic
SFSMS

SEQ ID NO: 162 - protein, 17 aa, synthetic construct
  REGION 1..17: note = Synthetic
SISGSSGTTYYADSVKG

SEQ ID NO: 163 - protein, 7 aa, synthetic construct
  REGION 1..7: note = Synthetic
PFPYFDY

SEQ ID NO: 164 - protein, 12 aa, synthetic construct
  REGION 1..12: note = Synthetic
RASQSVSSSFLA

SEQ ID NO: 165 - protein, 7 aa, synthetic construct
  REGION 1..7: note = Synthetic
YASSRAT

SEQ ID NO: 166 - protein, 9 aa, synthetic construct
  REGION 1..9: note = Synthetic
QQTGRIPPT

SEQ ID NO: 167-181 - skipped (15 entries)

SEQ ID NO: 182 - protein, 12 aa, synthetic construct
  REGION 1..12: note = Synthetic
GESPGGSSGSES

SEQ ID NO: 183 - protein, 12 aa, synthetic construct
  REGION 1..12: note = Synthetic
GSEGSSGPGESS

SEQ ID NO: 184 - protein, 12 aa, synthetic construct
  REGION 1..12: note = Synthetic
GSSESGSSEGGP

SEQ ID NO: 185 - protein, 12 aa, synthetic construct
  REGION 1..12: note = Synthetic
GSGGEPSESGSS

SEQ ID NO: 186 - protein, 12 aa, synthetic construct
  REGION 1..12: note = Synthetic
GSPAGSPTSTEE

SEQ ID NO: 187 - protein, 12 aa, synthetic construct
  REGION 1..12: note = Synthetic
GSEPATSGSETP

SEQ ID NO: 188 - protein, 12 aa, synthetic construct
  REGION 1..12: note = Synthetic
GTSESATPESGP

SEQ ID NO: 189 - protein, 12 aa, synthetic construct
  REGION 1..12: note = Synthetic
GTSTEPSEGSAP

SEQ ID NO: 190 - protein, 12 aa, synthetic construct
  REGION 1..12: note = Synthetic
GSTSESPSGTAP

SEQ ID NO: 191 - protein, 12 aa, synthetic construct
  REGION 1..12: note = Synthetic
GTSTPESGSASP

SEQ ID NO: 192 - protein, 12 aa, synthetic construct
  REGION 1..12: note = Synthetic
GTSPSGESSTAP

SEQ ID NO: 193 - protein, 12 aa, synthetic construct
  REGION 1..12: note = Synthetic
GSTSSTAESPGP

SEQ ID NO: 194 - protein, 12 aa, synthetic construct
  REGION 1..12: note = Synthetic
GTPGSGTASSSP

SEQ ID NO: 195 - protein, 12 aa, synthetic construct
  REGION 1..12: note = Synthetic
GSSTPSGATGSP

SEQ ID NO: 196 - protein, 12 aa, synthetic construct
  REGION 1..12: note = Synthetic
GSSPSASTGTGP

SEQ ID NO: 197 - protein, 12 aa, synthetic construct
  REGION 1..12: note = Synthetic
GASPGTSSTGSP

SEQ ID NO: 198 - protein, 12 aa, synthetic construct
  REGION 1..12: note = Synthetic
GEPAGSPTSTSE

SEQ ID NO: 199 - protein, 12 aa, synthetic construct
  REGION 1..12: note = Synthetic
GTGEPSSTPASE

SEQ ID NO: 200 - protein, 12 aa, synthetic construct
  REGION 1..12: note = Synthetic
GSGPSTESAPTE

SEQ ID NO: 201 - protein, 12 aa, synthetic construct
  REGION 1..12: note = Synthetic
GSETPSGPSETA

SEQ ID NO: 202 - protein, 12 aa, synthetic construct
  REGION 1..12: note = Synthetic
GPSETSTSEPGA

SEQ ID NO: 203 - protein, 12 aa, synthetic construct
  REGION 1..12: note = Synthetic
GSPSEPTEGTSA

SEQ ID NO: 204 - protein, 144 aa, synthetic construct
  REGION 1..144: note = Synthetic
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESATPESGPGSEPATSGSETPGTSTEPSEGSAP

SEQ ID NO: 205 - protein, 144 aa, synthetic construct
  REGION 1..144: note = Synthetic
GTSTPESGSASPGTSPSGESSTAPGTSPSGESSTAPGSTSSTAESPGPGSTSESPSGTAPGSTSSTAESPGPGTSPSGESSTAPGTSTPESGSASPGSTSSTAESPGPGTSPSGESSTAPGTSPSGESSTAPGTSPSGESSTAP

SEQ ID NO: 206 - protein, 288 aa, synthetic construct
  REGION 1..288: note = Synthetic
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 207 - protein, 504 aa, synthetic construct
  REGION 1..504: note = Synthetic
  VAR_SEQ 63: note = Xaa is any amino acid
  VAR_SEQ 207: note = Xaa is any amino acid
GASPGTSSTGSPGSSPSASTGTGPGSSPSASTGTGPGTPGSGTASSSPGSSTPSGATGSPGSXPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSXPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSP

SEQ ID NO: 208 - protein, 540 aa, synthetic construct
  REGION 1..540: note = Synthetic
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAP

SEQ ID NO: 209 - protein, 576 aa, synthetic construct
  REGION 1..576: note = Synthetic
GSSESGSSEGGPGSGGEPSESGSSGSSESGSSEGGPGSSESGSSEGGPGSSESGSSEGGPGSSESGSSEGGPGSSESGSSEGGPGESPGGSSGSESGSEGSSGPGESSGSSESGSSEGGPGSSESGSSEGGPGSSESGSSEGGPGSGGEPSESGSSGESPGGSSGSESGESPGGSSGSESGSGGEPSESGSSGSSESGSSEGGPGSGGEPSESGSSGSGGEPSESGSSGSEGSSGPGESSGESPGGSSGSESGSGGEPSESGSSGSGGEPSESGSSGSGGEPSESGSSGSSESGSSEGGPGESPGGSSGSESGESPGGSSGSESGESPGGSSGSESGESPGGSSGSESGESPGGSSGSESGSSESGSSEGGPGSGGEPSESGSSGSEGSSGPGESSGSSESGSSEGGPGSGGEPSESGSSGSSESGSSEGGPGSGGEPSESGSSGESPGGSSGSESGESPGGSSGSESGSSESGSSEGGPGSGGEPSESGSSGSSESGSSEGGPGSGGEPSESGSSGSGGEPSESGSSGESPGGSSGSESGSEGSSGPGESSGSSESGSSEGGPGSEGSSGPGESS

SEQ ID NO: 210 - protein, 576 aa, synthetic construct
  REGION 1..576: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 211 - protein, 576 aa, synthetic construct
  REGION 1..576: note = Synthetic
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAPGSTSSTAESPGPGTSTPESGSASPGTSTPESGSASP

SEQ ID NO: 212 - protein, 836 aa, synthetic construct
  REGION 1..836: note = Synthetic
GSSESGSSEGGPGSSESGSSEGGPGESPGGSSGSESGSGGEPSESGSSGESPGGSSGSESGESPGGSSGSESGSSESGSSEGGPGSSESGSSEGGPGSSESGSSEGGPGESPGGSSGSESGESPGGSSGSESGESPGGSSGSESGSSESGSSEGGPGSSESGSSEGGPGSSESGSSEGGPGSSESGSSEGGPGSSESGSSEGGPGSSESGSSEGGPGSGGEPSESGSSGESPGGSSGSESGESPGGSSGSESGSGGEPSESGSSGSEGSSGPGESSGSSESGSSEGGPGSGGEPSESGSSGSEGSSGPGESSGSSESGSSEGGPGSGGEPSESGSSGESPGGSSGSESGSGGEPSESGSSGSGGEPSESGSSGSSESGSSEGGPGSGGEPSESGSSGSGGEPSESGSSGSEGSSGPGESSGESPGGSSGSESGSEGSSGPGESSGSEGSSGPGESSGSGGEPSESGSSGSSESGSSEGGPGSSESGSSEGGPGESPGGSSGSESGSGGEPSESGSSGSEGSSGPGESSGESPGGSSGSESGSEGSSGPGSSESGSSEGGPGSGGEPSESGSSGSEGSSGPGESSGSEGSSGPGESSGSEGSSGPGESSGSGGEPSESGSSGSGGEPSESGSSGESPGGSSGSESGESPGGSSGSESGSGGEPSESGSSGSEGSSGPGESSGESPGGSSGSESGSSESGSSEGGPGSSESGSSEGGPGSSESGSSEGGPGSGGEPSESGSSGSSESGSSEGGPGESPGGSSGSESGSGGEPSESGSSGSSESGSSEGGPGESPGGSSGSESGSGGEPSESGSSGESPGGSSGSESGSGGEPSESGSS

SEQ ID NO: 213 - protein, 864 aa, synthetic construct
  REGION 1..864: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 214 - protein, 875 aa, synthetic construct
  REGION 1..875: note = Synthetic
  VAR_SEQ 430..432: note = Xaa is any amino acid
  VAR_SEQ 443..446: note = Xaa is any amino acid
GSTSESPSGTAPGTSPSGESSTAPGSTSESPSGTAPGSTSESPSGTAPGTSTPESGSASPGTSTPESGSASPGSTSESPSGTAPGSTSESPSGTAPGTSPSGESSTAPGSTSESPSGTAPGTSPSGESSTAPGTSPSGESSTAPGSTSSTAESPGPGTSPSGESSTAPGTSPSGESSTAPGSTSSTAESPGPGTSTPESGSASPGTSTPESGSASPGSTSESPSGTAPGSTSESPSGTAPGTSTPESGSASPGSTSSTAESPGPGTSTPESGSASPGSTSESPSGTAPGTSPSGESSTAPGSTSSTAESPGPGTSPSGESSTAPGTSTPESGSASPGSTSSTAESPGPGSTSSTAESPGPGSTSSTAESPGPGSTSSTAESPGPGTSPSGESSTAPGSTSESPSGTAPGSTSESPSGTAPGTSTPESGPXXXGASASGAPSTXXXXSESPSGTAPGSTSESPSGTAPGSTSESPSGTAPGSTSESPSGTAPGSTSESPSGTAPGSTSESPSGTAPGTSTPESGSASPGTSPSGESSTAPGTSPSGESSTAPGSTSSTAESPGPGTSPSGESSTAPGTSTPESGSASPGSTSESPSGTAPGSTSESPSGTAPGTSPSGESSTAPGSTSESPSGTAPGTSTPESGSASPGTSTPESGSASPGSTSESPSGTAPGTSTPESGSASPGSTSSTAESPGPGSTSESPSGTAPGSTSESPSGTAPGTSPSGESSTAPGSTSSTAESPGPGTSPSGESSTAPGTSTPESGSASPGTSPSGESSTAPGTSPSGESSTAPGTSPSGESSTAPGSTSSTAESPGPGSTSSTAESPGPGTSPSGESSTAPGSSPSASTGTGPGSSTPSGATGSPGSSTPSGATGSP

SEQ ID NO: 215 - protein, 864 aa, synthetic construct
  REGION 1..864: note = Synthetic
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 216 - protein, 875 aa, synthetic construct
  REGION 1..875: note = Synthetic
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 217 - protein, 913 aa, synthetic construct
  REGION 1..913: note = Synthetic
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 218 - protein, 924 aa, synthetic construct
  REGION 1..924: note = Synthetic
MAEPAGSPTSTEEGASPGTSSTGSPGSSTPSGATGSPGSSTPSGATGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 219 - protein, 1318 aa, synthetic construct
  REGION 1..1318: note = Synthetic
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGPEPTGPAPSGGSEPATSGSETPGTSESATPESGPGSPAGSPTSTEEGTSESATPESGPGSPAGSPTSTEEGSPAGSPTSTEEGTSESATPESGPGSPAGSPTSTEEGSPAGSPTSTEEGSTSSTAESPGPGSTSESPSGTAPGTSPSGESSTAPGSTSESPSGTAPGSTSESPSGTAPGTSPSGESSTAPGTSTEPSEGSAPGTSESATPESGPGTSESATPESGPGSEPATSGSETPGTSESATPESGPGTSESATPESGPGTSTEPSEGSAPGTSESATPESGPGTSTEPSEGSAPGTSPSGESSTAPGTSPSGESSTAPGTSPSGESSTAPGTSTEPSEGSAPGSPAGSPTSTEEGTSTEPSEGSAPGSSPSASTGTGPGSSTPSGATGSPGSSTPSGATGSPGSSTPSGATGSPGSSTPSGATGSPGASPGTSSTGSPGASASGAPSTGGTSPSGESSTAPGSTSSTAESPGPGTSPSGESSTAPGTSESATPESGPGTSTEPSEGSAPGTSTEPSEGSAPGSSPSASTGTGPGSSTPSGATGSPGASPGTSSTGSPGTSTPESGSASPGTSPSGESSTAPGTSPSGESSTAPGTSESATPESGPGSEPATSGSETPGTSTEPSEGSAPGSTSESPSGTAPGSTSESPSGTAPGTSTPESGSASPGSPAGSPTSTEEGTSESATPESGPGTSTEPSEGSAPGSPAGSPTSTEEGTSESATPESGPGSEPATSGSETPGSSTPSGATGSPGASPGTSSTGSPGSSTPSGATGSPGSTSESPSGTAPGTSPSGESSTAPGSTSSTAESPGPGSSTPSGATGSPGASPGTSSTGSPGTPGSGTASSSPGSPAGSPTSTEEGSPAGSPTSTEEGTSTEPSEGSAP

SEQ ID NO: 220 - protein, 864 aa, synthetic construct
  REGION 1..864: note = Synthetic
GTSTEPSEPGSAGTSTEPSEPGSAGSEPATSGTEPSGSGASEPTSTEPGSEPATSGTEPSGSEPATSGTEPSGSEPATSGTEPSGSGASEPTSTEPGTSTEPSEPGSAGSEPATSGTEPSGTSTEPSEPGSAGSEPATSGTEPSGSEPATSGTEPSGTSTEPSEPGSAGTSTEPSEPGSAGSEPATSGTEPSGSEPATSGTEPSGTSEPSTSEPGAGSGASEPTSTEPGTSEPSTSEPGAGSEPATSGTEPSGSEPATSGTEPSGTSTEPSEPGSAGTSTEPSEPGSAGSGASEPTSTEPGSEPATSGTEPSGSEPATSGTEPSGSEPATSGTEPSGSEPATSGTEPSGTSTEPSEPGSAGSEPATSGTEPSGSGASEPTSTEPGTSTEPSEPGSAGSEPATSGTEPSGSGASEPTSTEPGTSTEPSEPGSAGSGASEPTSTEPGSEPATSGTEPSGSGASEPTSTEPGSEPATSGTEPSGSGASEPTSTEPGTSTEPSEPGSAGSEPATSGTEPSGSGASEPTSTEPGTSTEPSEPGSAGSEPATSGTEPSGTSTEPSEPGSAGSEPATSGTEPSGTSTEPSEPGSAGTSTEPSEPGSAGTSTEPSEPGSAGTSTEPSEPGSAGTSTEPSEPGSAGTSTEPSEPGSAGTSEPSTSEPGAGSGASEPTSTEPGTSTEPSEPGSAGTSTEPSEPGSAGTSTEPSEPGSAGSEPATSGTEPSGSGASEPTSTEPGSEPATSGTEPSGSEPATSGTEPSGSEPATSGTEPSGSEPATSGTEPSGTSEPSTSEPGAGSEPATSGTEPSGSGASEPTSTEPGTSTEPSEPGSAGSEPATSGTEPSGSGASEPTSTEPGTSTEPSEPGSA

SEQ ID NO: 221 - protein, 864 aa, synthetic construct
  REGION 1..864: note = Synthetic
GSETATSGSETAGTSESATSESGAGSTAGSETSTEAGTSESATSESGAGSETATSGSETAGSETATSGSETAGTSTEASEGSASGTSTEASEGSASGTSESATSESGAGSETATSGSETAGTSTEASEGSASGSTAGSETSTEAGTSESATSESGAGTSESATSESGAGSETATSGSETAGTSESATSESGAGTSTEASEGSASGSETATSGSETAGSETATSGSETAGTSTEASEGSASGSTAGSETSTEAGTSESATSESGAGTSTEASEGSASGSETATSGSETAGSTAGSETSTEAGSTAGSETSTEAGSETATSGSETAGTSESATSESGAGTSESATSESGAGSETATSGSETAGTSESATSESGAGTSESATSESGAGSETATSGSETAGSETATSGSETAGTSTEASEGSASGSTAGSETSTEAGSETATSGSETAGTSESATSESGAGSTAGSETSTEAGSTAGSETSTEAGSTAGSETSTEAGTSTEASEGSASGSTAGSETSTEAGSTAGSETSTEAGTSTEASEGSASGSTAGSETSTEAGSETATSGSETAGTSTEASEGSASGTSESATSESGAGSETATSGSETAGTSESATSESGAGTSESATSESGAGSETATSGSETAGTSESATSESGAGSETATSGSETAGTSTEASEGSASGTSTEASEGSASGSTAGSETSTEAGSTAGSETSTEAGSETATSGSETAGTSESATSESGAGTSESATSESGAGSETATSGSETAGSETATSGSETAGSETATSGSETAGTSTEASEGSASGTSESATSESGAGSETATSGSETAGSETATSGSETAGTSESATSESGAGTSESATSESGAGSETATSGSETA

SEQ ID NO: 222-246 - skipped (25 entries)

SEQ ID NO: 247 - DNA, 432 bp, synthetic construct
  misc_feature 1..432: note = Synthetic
  source 1..432: mol_type = other DNA
ggtagcgaaccggcaacttccggctctgaaaccccaggtacttctgaaagcgctactcctgagtctggcccaggtagcgaacctgctacctctggctctgaaaccccaggtagcccggcaggctctccgacttccaccgaggaaggtacctctactgaaccttctgagggtagcgctccaggtagcgaaccggcaacctctggctctgaaaccccaggtagcgaacctgctacctccggctctgaaactccaggtagcgaaccggctacttccggttctgaaactccaggtacctctaccgaaccttccgaaggcagcgcaccaggtacttctgaaagcgcaacccctgaatccggtccaggtagcgaaccggctacttctggctctgagactccaggtacttctaccgaaccgtccgaaggtagcgcacca

SEQ ID NO: 248 - DNA, 432 bp, synthetic construct
  misc_feature 1..432: note = Synthetic
  source 1..432: mol_type = other DNA
ggtacttctactccggaaagcggttccgcatctccaggtacttctcctagcggtgaatcttctactgctccaggtacctctcctagcggcgaatcttctactgctccaggttctaccagctctaccgctgaatctcctggcccaggttctaccagcgaatccccgtctggcaccgcaccaggttctactagctctaccgcagaatctccgggtccaggtacttcccctagcggtgaatcttctactgctccaggtacctctactccggaaagcggctccgcatctccaggttctactagctctactgctgaatctcctggtccaggtacctcccctagcggcgaatcttctactgctccaggtacctctcctagcggcgaatcttctaccgctccaggtacctcccctagcggtgaatcttctaccgcacca

SEQ ID NO: 249 - DNA, 864 bp, synthetic construct
  misc_feature 1..864: note = Synthetic
  source 1..864: mol_type = other DNA
ggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 250 - DNA, 1728 bp, synthetic construct
  misc_feature 1..1728: note = Synthetic
  source 1..1728: mol_type = other DNA
ggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcacca

SEQ ID NO: 251 - DNA, 1728 bp, synthetic construct
  misc_feature 1..1728: note = Synthetic
  source 1..1728: mol_type = other DNA
ggttctactagctctaccgctgaatctcctggcccaggttccactagctctaccgcagaatctccgggcccaggttctactagcgaatccccttctggtaccgctccaggttctactagctctaccgctgaatctccgggtccaggttctaccagctctactgcagaatctcctggcccaggtacttctactccggaaagcggttccgcttctccaggttctaccagcgaatctccttctggcaccgctccaggtacctctcctagcggcgaatcttctaccgctccaggttctactagcgaatctccttctggcactgcaccaggttctaccagcgaatctccttctggcaccgctccaggtacctctcctagcggcgaatcttctaccgctccaggttctactagcgaatctccttctggcactgcaccaggttctaccagcgaatctccttctggcaccgctccaggtacctctcctagcggcgaatcttctaccgctccaggttctactagcgaatctccttctggcactgcaccaggttctactagcgaatctccttctggcactgcaccaggttctaccagcgaatctccgtctggcactgcaccaggtacctctacccctgaaagcggttccgcttctccaggttctactagcgaatctccttctggtaccgctccaggtacttctacccctgaaagcggctccgcttctccaggttccactagctctaccgctgaatctccgggtccaggttctactagctctactgcagaatctcctggcccaggtacctctactccggaaagcggctctgcatctccaggtacttctacccctgaaagcggttctgcatctccaggttctactagcgaatccccgtctggtaccgcaccaggtacttctaccccggaaagcggctctgcttctccaggtacttctaccccggaaagcggctccgcatctccaggttctactagcgaatctccttctggtaccgctccaggttctaccagcgaatccccgtctggtactgctccaggttctaccagcgaatctccttctggtactgcaccaggttctactagctctactgcagaatctcctggcccaggtacctctactccggaaagcggctctgcatctccaggtacttctacccctgaaagcggttctgcatctccaggttctactagcgaatctccttctggcactgcaccaggttctaccagcgaatctccgtctggcactgcaccaggtacctctacccctgaaagcggttccgcttctccaggttctactagcgaatctccttctggcactgcaccaggttctaccagcgaatctccgtctggcactgcaccaggtacctctacccctgaaagcggttccgcttctccaggtacttctccgagcggtgaatcttctaccgcaccaggttctactagctctaccgctgaatctccgggcccaggtacttctccgagcggtgaatcttctactgctccaggttccactagctctactgctgaatctcctggcccaggtacttctactccggaaagcggttccgcttctccaggttctactagcgaatctccgtctggcaccgcaccaggttctactagctctactgcagaatctcctggcccaggtacctctactccggaaagcggctctgcatctccaggtacttctacccctgaaagcggttctgcatctcca

SEQ ID NO: 252 - DNA, 2625 bp, synthetic construct
  misc_feature 1..2625: note = Synthetic
  source 1..2625: mol_type = other DNA
ggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcacca

SEQ ID NO: 253 - DNA, 2592 bp, synthetic construct
  misc_feature 1..2592: note = Synthetic
  source 1..2592: mol_type = other DNA
ggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 254 - DNA, 2624 bp, synthetic construct
  misc_feature 1..2624: note = Synthetic
  misc_feature 1287..1298: note = N can be an nucleotide
  misc_feature 1329..1337: note = N can be an nucleotide
  source 1..2624: mol_type = other DNA
ggttctaccagcgaatctccttctggcaccgctccaggtacctctcctagcggcgaatcttctaccgctccaggttctactagcgaatctccttctggcactgcaccaggttctactagcgaatccccgtctggtactgctccaggtacttctactcctgaaagcggttccgcttctccaggtacctctactccggaaagcggttctgcatctccaggttctaccagcgaatctccttctggcaccgctccaggttctactagcgaatccccgtctggtaccgcaccaggtacttctcctagcggcgaatcttctaccgcaccaggttctactagcgaatctccgtctggcactgctccaggtacttctcctagcggtgaatcttctaccgctccaggtacttcccctagcggcgaatcttctaccgctccaggttctactagctctactgcagaatctccgggcccaggtacctctcctagcggtgaatcttctaccgctccaggtacttctccgagcggtgaatcttctaccgctccaggttctactagctctactgcagaatctcctggcccaggtacctctactccggaaagcggctctgcatctccaggtacttctacccctgaaagcggttctgcatctccaggttctactagcgaatctccttctggcactgcaccaggttctaccagcgaatctccgtctggcactgcaccaggtacctctacccctgaaagcggttccgcttctccaggttctaccagctctaccgcagaatctcctggtccaggtacctctactccggaaagcggctctgcatctccaggttctactagcgaatctccttctggcactgcaccaggtacttctccgagcggtgaatcttctaccgcaccaggttctactagctctaccgctgaatctccgggcccaggtacttctccgagcggtgaatcttctactgctccaggtacctctactcctgaaagcggttctgcatctccaggttccactagctctaccgcagaatctccgggcccaggttctactagctctactgctgaatctcctggcccaggttctactagctctactgctgaatctccgggtccaggttctaccagctctactgctgaatctcctggtccaggtacctccccgagcggtgaatcttctactgcaccaggttctactagcgaatctccttctggcactgcaccaggttctaccagcgaatctccgtctggcactgcaccaggtacctctacccctgaaagcggtccnnnnnnnnnnntgcaagcgcaagcggcgcgccaagcacgggannnnnnnntagcgaatctccttctggtaccgctccaggttctaccagcgaatccccgtctggtactgctccaggttctaccagcgaatctccttctggtactgcaccaggttctactagcgaatctccttctggtaccgctccaggttctaccagcgaatccccgtctggtactgctccaggttctaccagcgaatctccttctggtactgcaccaggtacttctactccggaaagcggttccgcatctccaggtacttctcctagcggtgaatcttctactgctccaggtacctctcctagcggcgaatcttctactgctccaggttctaccagctctactgctgaatctccgggtccaggtacttccccgagcggtgaatcttctactgcaccaggtacttctactccggaaagcggttccgcttctccaggttctaccagcgaatctccttctggcaccgctccaggttctactagcgaatccccgtctggtaccgcaccaggtacttctcctagcggcgaatcttctaccgcaccaggttctactagcgaatccccgtctggtaccgcaccaggtacttctaccccggaaagcggctctgcttctccaggtacttctaccccggaaagcggctccgcatctccaggttctactagcgaatctccttctggtaccgctccaggtacttctacccctgaaagcggctccgcttctccaggttccactagctctaccgctgaatctccgggtccaggttctaccagcgaatctccttctggcaccgctccaggttctactagcgaatccccgtctggtaccgcaccaggtacttctcctagcggcgaatcttctaccgcaccaggttctaccagctctactgctgaatctccgggtccaggtacttccccgagcggtgaatcttctactgcaccaggtacttctactccggaaagcggttccgcttctccaggtacctcccctagcggcgaatcttctactgctccaggtacctctcctagcggcgaatcttctaccgctccaggtacctcccctagcggtgaatcttctaccgcaccaggttctactagctctactgctgaatctccgggtccaggttctaccagctctactgctgaatctcctggtccaggtacctccccgagcggtgaatcttctactgcaccaggttctagcccttctgcttccaccggtaccggcccaggtagctctactccgtctggtgcaactggctctccaggtagctctactccgtctggtgcaaccggctcccca

SEQ ID NO: 255 - DNA, 2592 bp, synthetic construct
  misc_feature 1..2592: note = Synthetic
  source 1..2592: mol_type = other DNA
ggtgcttccccgggcaccagctctactggttctccaggttctagcccgtctgcttctactggtactggtccaggttctagcccttctgcttccactggtactggtccaggtaccccgggtagcggtaccgcttcttcttctccaggtagctctactccgtctggtgctaccggctctccaggttctaacccttctgcatccaccggtaccggcccaggtgcttctccgggcaccagctctactggttctccaggtaccccgggcagcggtaccgcatcttcttctccaggtagctctactccttctggtgcaactggttctccaggtactcctggcagcggtaccgcttcttcttctccaggtgcttctcctggtactagctctactggttctccaggtgcttctccgggcactagctctactggttctccaggtaccccgggtagcggtactgcttcttcctctccaggtagctctaccccttctggtgcaaccggctctccaggtgcttctccgggcaccagctctaccggttctccaggtaccccgggtagcggtaccgcttcttcttctccaggtagctctactccgtctggtgctaccggctctccaggttctaacccttctgcatccaccggtaccggcccaggttctagcccttctgcttccaccggtactggcccaggtagctctaccccttctggtgctaccggctccccaggtagctctactccttctggtgcaactggctctccaggtgcatctccgggcactagctctactggttctccaggtgcatcccctggcactagctctactggttctccaggtgcttctcctggtaccagctctactggttctccaggtactcctggcagcggtaccgcttcttcttctccaggtgcttctcctggtactagctctactggttctccaggtgcttctccgggcactagctctactggttctccaggtgcttccccgggcactagctctaccggttctccaggttctagcccttctgcatctactggtactggcccaggtactccgggcagcggtactgcttcttcctctccaggtgcatctccgggcactagctctactggttctccaggtgcatcccctggcactagctctactggttctccaggtgcttctcctggtaccagctctactggttctccaggtagctctactccgtctggtgcaaccggttccccaggtagctctactccttctggtgctactggctccccaggtgcatcccctggcaccagctctaccggttctccaggtaccccgggcagcggtaccgcatcttcctctccaggtagctctaccccgtctggtgctaccggttccccaggtagctctaccccgtctggtgcaaccggctccccaggtagctctactccgtctggtgcaaccggctccccaggttctagcccgtctgcttccactggtactggcccaggtgcttccccgggcaccagctctactggttctccaggtgcatccccgggtaccagctctaccggttctccaggtactcctggcagcggtactgcatcttcctctccaggtgcttctccgggcaccagctctactggttctccaggtgcatctccgggcactagctctactggttctccaggtgcatcccctggcactagctctactggttctccaggtgcttctcctggtaccagctctactggttctccaggtacccctggtagcggtactgcttcttcctctccaggtagctctactccgtctggtgctaccggttctccaggtaccccgggtagcggtaccgcatcttcttctccaggtagctctaccccgtctggtgctactggttctccaggtactccgggcagcggtactgcttcttcctctccaggtagctctaccccttctggtgctactggctctccaggtagctctaccccgtctggtgctactggctccccaggttctagcccttctgcatccaccggtaccggtccaggttctagcccgtctgcatctactggtactggtccaggtgcatccccgggcactagctctaccggttctccaggtactcctggtagcggtactgcttcttcttctccaggtagctctactccttctggtgctactggttctccaggttctagcccttctgcatccaccggtaccggcccaggttctagcccgtctgcttctaccggtactggtccaggtgcttctccgggtactagctctactggttctccaggtgcatctcctggtactagctctactggttctccaggtagctctactccgtctggtgcaaccggctctccaggttctagcccttctgcatctaccggtactggtccaggtgcatcccctggtaccagctctaccggttctccaggttctagcccttctgcttctaccggtaccggtccaggtacccctggcagcggtaccgcatcttcctctccaggtagctctactccgtctggtgcaaccggttccccaggtagctctactccttctggtgctactggctccccaggtgcatcccctggcaccagctctaccggttctcca

SEQ ID NO: 256 - DNA, 2772 bp, synthetic construct
  misc_feature 1..2772: note = Synthetic
  source 1..2772: mol_type = other DNA
atggctgaacctgctggctctccaacctccactgaggaaggtgcatccccgggcaccagctctaccggttctccaggtagctctaccccgtctggtgctaccggctctccaggtagctctaccccgtctggtgctactggctctccaggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcacca

SEQ ID NO: 257 - DNA, 2739 bp, synthetic construct
  misc_feature 1..2739: note = Synthetic
  source 1..2739: mol_type = other DNA
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 258 - DNA, 3954 bp, synthetic construct
  misc_feature 1..3954: note = Synthetic
  source 1..3954: mol_type = other DNA
ggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtccagaaccaacggggccggccccaagcggaggtagcgaaccggcaacctccggctctgaaaccccaggtacctctgaaagcgctactcctgaatccggcccaggtagcccggcaggttctccgacttccactgaggaaggtacttctgaaagcgctactcctgagtccggcccaggtagcccggctggctctccgacttccaccgaggaaggtagcccggctggctctccaacttctactgaagaaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggttctaccagcgaatctccttctggcaccgctccaggttctactagcgaatccccgtctggtaccgcaccaggtacttctcctagcggcgaatcttctaccgcaccaggtacttctaccgaaccttccgagggcagcgcaccaggtacttctgaaagcgctacccctgagtccggcccaggtacttctgaaagcgctactcctgaatccggtccaggtagcgaaccggcaacctctggctctgaaaccccaggtacctctgaaagcgctactccggaatctggtccaggtacttctgaaagcgctactccggaatccggtccaggtacctctactgaaccttctgagggcagcgctccaggtacttctgaaagcgctaccccggagtccggtccaggtacttctactgaaccgtccgaaggtagcgcaccaggtacctcccctagcggcgaatcttctactgctccaggtacctctcctagcggcgaatcttctaccgctccaggtacctcccctagcggtgaatcttctaccgcaccaggtacttctaccgaaccgtccgagggtagcgcaccaggtagcccagcaggttctcctacctccaccgaggaaggtacttctaccgaaccgtccgagggtagcgcaccaggttctagcccttctgcttccaccggtaccggcccaggtagctctactccgtctggtgcaactggctctccaggtagctctactccgtctggtgcaaccggctccccaggtagctctaccccgtctggtgctaccggctctccaggtagctctaccccgtctggtgcaaccggctccccaggtgcatccccgggtactagctctaccggttctccaggtgcaagcgcaagcggcgcgccaagcacgggaggtacttctccgagcggtgaatcttctaccgcaccaggttctactagctctaccgctgaatctccgggcccaggtacttctccgagcggtgaatcttctactgctccaggtacctctgaaagcgctactccggagtctggcccaggtacctctactgaaccgtctgagggtagcgctccaggtacttctactgaaccgtccgaaggtagcgcaccaggttctagcccttctgcatctactggtactggcccaggtagctctactccttctggtgctaccggctctccaggtgcttctccgggtactagctctaccggttctccaggtacttctactccggaaagcggttccgcatctccaggtacttctcctagcggtgaatcttctactgctccaggtacctctcctagcggcgaatcttctactgctccaggtacttctgaaagcgcaacccctgaatccggtccaggtagcgaaccggctacttctggctctgagactccaggtacttctaccgaaccgtccgaaggtagcgcaccaggttctaccagcgaatccccttctggtactgctccaggttctaccagcgaatccccttctggcaccgcaccaggtacttctacccctgaaagcggctccgcttctccaggtagcccggcaggctctccgacctctactgaggaaggtacttctgaaagcgcaaccccggagtccggcccaggtacctctaccgaaccgtctgagggcagcgcaccaggtagccctgctggctctccaacctccaccgaagaaggtacctctgaaagcgcaacccctgaatccggcccaggtagcgaaccggcaacctccggttctgaaaccccaggtagctctaccccgtctggtgctaccggttccccaggtgcttctcctggtactagctctaccggttctccaggtagctctaccccgtctggtgctactggctctccaggttctactagcgaatccccgtctggtactgctccaggtacttcccctagcggtgaatcttctactgctccaggttctaccagctctaccgcagaatctccgggtccaggtagctctaccccttctggtgcaaccggctctccaggtgcatccccgggtaccagctctaccggttctccaggtactccgggtagcggtaccgcttcttcctctccaggtagccctgctggctctccgacttctactgaggaaggtagcccggctggttctccgacttctactgaggaaggtacttctaccgaaccttccgaaggtagcgctcca

SEQ ID NO: 259 - DNA, 2592 bp, synthetic construct
  misc_feature 1..2592: note = Synthetic
  source 1..2592: mol_type = other DNA
ggtacttccaccgaaccatccgaaccaggtagcgcaggtacttccaccgaaccatccgaacctggcagcgcaggtagcgaaccggcaacctctggtactgaaccatcaggtagcggcgcatccgagcctacctctactgaaccaggtagcgaaccggctacctccggtactgagccatcaggtagcgaaccggcaacttccggtactgaaccatcaggtagcgaaccggcaacttccggcactgaaccatcaggtagcggtgcatctgagccgacctctactgaaccaggtacttctactgaaccatctgagccgggcagcgcaggtagcgaaccagctacttctggcactgaaccatcaggtacttctactgaaccatccgaaccaggtagcgcaggtagcgaacctgctacctctggtactgagccatcaggtagcgaaccggctacctctggtactgaaccatcaggtacttctaccgaaccatccgagcctggtagcgcaggtacttctaccgaaccatccgagccaggcagcgcaggtagcgaaccggcaacctctggcactgagccatcaggtagcgaaccagcaacttctggtactgaaccatcaggtactagcgagccatctacttccgaaccaggtgcaggtagcggcgcatccgaacctacttccactgaaccaggtactagcgagccatccacctctgaaccaggtgcaggtagcgaaccggcaacttccggcactgaaccatcaggtagcgaaccggctacctctggtactgaaccatcaggtacttctaccgaaccatccgagcctggtagcgcaggtacttctaccgaaccatccgagccaggcagcgcaggtagcggtgcatccgagccgacctctactgaaccaggtagcgaaccagcaacttctggcactgagccatcaggtagcgaaccagctacctctggtactgaaccatcaggtagcgaaccggctacttccggcactgaaccatcaggtagcgaaccagcaacctccggtactgaaccatcaggtacttccactgaaccatccgaaccgggtagcgcaggtagcgaaccggcaacttccggcactgaaccatcaggtagcggtgcatctgagccgacctctactgaaccaggtacttctactgaaccatctgagccgggcagcgcaggtagcgaacctgcaacctccggcactgagccatcaggtagcggcgcatctgaaccaacctctactgaaccaggtacttccaccgaaccatctgagccaggcagcgcaggtagcggcgcatctgaaccaacctctactgaaccaggtagcgaaccagcaacttctggtactgaaccatcaggtagcggcgcatctgagcctacttccactgaaccaggtagcgaaccggcaacttccggcactgaaccatcaggtagcggtgcatctgagccgacctctactgaaccaggtacttctactgaaccatctgagccgggcagcgcaggtagcgaaccggcaacttccggcactgaaccatcaggtagcggtgcatctgagccgacctctactgaaccaggtacttctactgaaccatctgagccgggcagcgcaggtagcgaaccagctacttctggcactgaaccatcaggtacttctactgaaccatccgaaccaggtagcgcaggtagcgaacctgctacctctggtactgagccatcaggtacttctactgaaccatccgagccgggtagcgcaggtacttccactgaaccatctgaacctggtagcgcaggtacttccactgaaccatccgaaccaggtagcgcaggtacttctactgaaccatccgagccgggtagcgcaggtacttccactgaaccatctgaacctggtagcgcaggtacttccactgaaccatccgaaccaggtagcgcaggtactagcgaaccatccacctccgaaccaggcgcaggtagcggtgcatctgaaccgacttctactgaaccaggtacttccactgaaccatctgagccaggtagcgcaggtacttccaccgaaccatccgaaccaggtagcgcaggtacttccaccgaaccatccgaacctggcagcgcaggtagcgaaccggcaacctctggtactgaaccatcaggtagcggtgcatccgagccgacctctactgaaccaggtagcgaaccagcaacttctggcactgagccatcaggtagcgaaccagctacctctggtactgaaccatcaggtagcgaaccggcaacctctggcactgagccatcaggtagcgaaccagcaacttctggtactgaaccatcaggtactagcgagccatctacttccgaaccaggtgcaggtagcgaacctgcaacctccggcactgagccatcaggtagcggcgcatctgaaccaacctctactgaaccaggtacttccaccgaaccatctgagccaggcagcgcaggtagcgaacctgcaacctccggcactgagccatcaggtagcggcgcatctgaaccaacctctactgaaccaggtacttccaccgaaccatctgagccaggcagcgca

SEQ ID NO: 260 - DNA, 2592 bp, synthetic construct
  misc_feature 1..2592: note = Synthetic
  source 1..2592: mol_type = other DNA
ggtagcgaaactgctacttccggctctgagactgcaggtactagtgaatccgcaactagcgaatctggcgcaggtagcactgcaggctctgagacttccactgaagcaggtactagcgagtccgcaaccagcgaatccggcgcaggtagcgaaactgctacctctggctccgagactgcaggtagcgaaactgcaacctctggctctgaaactgcaggtacttccactgaagcaagtgaaggctccgcatcaggtacttccaccgaagcaagcgaaggctccgcatcaggtactagtgagtccgcaactagcgaatccggtgcaggtagcgaaaccgctacctctggttccgaaactgcaggtacttctaccgaggctagcgaaggttctgcatcaggtagcactgctggttccgagacttctactgaagcaggtactagcgaatctgctactagcgaatccggcgcaggtactagcgaatccgctaccagcgaatccggcgcaggtagcgaaactgcaacctctggttccgagactgcaggtactagcgagtccgctactagcgaatctggcgcaggtacttccactgaagctagtgaaggttctgcatcaggtagcgaaactgctacttctggttccgaaactgcaggtagcgaaaccgctacctctggttccgaaactgcaggtacttctaccgaggctagcgaaggttctgcatcaggtagcactgctggttccgagacttctactgaagcaggtactagcgagtccgctactagcgaatctggcgcaggtacttccactgaagctagtgaaggttctgcatcaggtagcgaaactgctacttctggttccgaaactgcaggtagcactgctggctccgagacttctaccgaagcaggtagcactgcaggttccgaaacttccactgaagcaggtagcgaaactgctacctctggctctgagactgcaggtactagcgaatctgctactagcgaatccggcgcaggtactagcgaatccgctaccagcgaatccggcgcaggtagcgaaactgcaacctctggttccgagactgcaggtactagcgaatctgctactagcgaatccggcgcaggtactagcgaatccgctaccagcgaatccggcgcaggtagcgaaactgcaacctctggttccgagactgcaggtagcgaaaccgctacctctggttccgaaactgcaggtacttctaccgaggctagcgaaggttctgcatcaggtagcactgctggttccgagacttctactgaagcaggtagcgaaactgctacttccggctctgagactgcaggtactagtgaatccgcaactagcgaatctggcgcaggtagcactgcaggctctgagacttccactgaagcaggtagcactgctggttccgaaacctctaccgaagcaggtagcactgcaggttctgaaacctccactgaagcaggtacttccactgaggctagtgaaggctctgcatcaggtagcactgctggttccgaaacctctaccgaagcaggtagcactgcaggttctgaaacctccactgaagcaggtacttccactgaggctagtgaaggctctgcatcaggtagcactgcaggttctgagacttccaccgaagcaggtagcgaaactgctacttctggttccgaaactgcaggtacttccactgaagctagtgaaggttccgcatcaggtactagtgagtccgcaaccagcgaatccggcgcaggtagcgaaaccgcaacctccggttctgaaactgcaggtactagcgaatccgcaaccagcgaatctggcgcaggtactagtgagtccgcaaccagcgaatccggcgcaggtagcgaaaccgcaacctccggttctgaaactgcaggtactagcgaatccgcaaccagcgaatctggcgcaggtagcgaaactgctacttccggctctgagactgcaggtacttccaccgaagcaagcgaaggttccgcatcaggtacttccaccgaggctagtgaaggctctgcatcaggtagcactgctggctccgagacttctaccgaagcaggtagcactgcaggttccgaaacttccactgaagcaggtagcgaaactgctacctctggctctgagactgcaggtactagcgaatctgctactagcgaatccggcgcaggtactagcgaatccgctaccagcgaatccggcgcaggtagcgaaactgcaacctctggttccgagactgcaggtagcgaaactgctacttccggctccgagactgcaggtagcgaaactgctacttctggctccgaaactgcaggtacttctactgaggctagtgaaggttccgcatcaggtactagcgagtccgcaaccagcgaatccggcgcaggtagcgaaactgctacctctggctccgagactgcaggtagcgaaactgcaacctctggctctgaaactgcaggtactagcgaatctgctactagcgaatccggcgcaggtactagcgaatccgctaccagcgaatccggcgcaggtagcgaaactgcaacctctggttccgagactgca

SEQ ID NO: 261 - DNA, 37 bp, synthetic construct
  misc_feature 1..37: note = Synthetic
  source 1..37: mol_type = other DNA
aggtgcaagcgcaagcggcgcgccaagcacgggaggt

SEQ ID NO: 262 - DNA, 37 bp, synthetic construct
  misc_feature 1..37: note = Synthetic
  source 1..37: mol_type = other DNA
aggtccagaaccaacggggccggccccaagcggaggt

SEQ ID NO: 263 - protein, 6 aa, synthetic construct
  REGION 1..6: note = Synthetic
HHHHHH

SEQ ID NO: 264 - protein, 4 aa, synthetic construct
  REGION 1..4: note = Synthetic
DDDK

SEQ ID NO: 265 - protein, 4 aa, synthetic construct
  REGION 1..4: note = Synthetic
IDGR

SEQ ID NO: 266 - protein, 6 aa, synthetic construct
  REGION 1..6: note = Synthetic
LVPRGS

SEQ ID NO: 267 - protein, 8 aa, synthetic construct
  REGION 1..8: note = Synthetic
LEVLFQGP

SEQ ID NO: 268 - protein, 7 aa, synthetic construct
  REGION 1..7: note = Synthetic
EQLYFQG

SEQ ID NO: 269 - protein, 7 aa, synthetic construct
  REGION 1..7: note = Synthetic
ETLFQGP

SEQ ID NO: 270 - protein, 4 aa, synthetic construct
  REGION 1..4: note = Synthetic
GGSG

SEQ ID NO: 271 - protein, 9 aa, synthetic construct
  REGION 1..9: note = Synthetic
FDKLPRTSG

SEQ ID NO: 272 - protein, 7 aa, synthetic construct
  REGION 1..7: note = Synthetic
FKLMVWY

SEQ ID NO: 273 - protein, 5 aa, synthetic construct
  REGION 1..5: note = Synthetic
GGGGS

SEQ ID NO: 274 - protein, 8 aa, synthetic construct
  REGION 1..8: note = Synthetic
KLTRVVGG

SEQ ID NO: 275 - protein, 8 aa, synthetic construct
  REGION 1..8: note = Synthetic
TMTRIVGG

SEQ ID NO: 276 - protein, 8 aa, synthetic construct
  REGION 1..8: note = Synthetic
SPFRSTGG

SEQ ID NO: 277 - protein, 8 aa, synthetic construct
  REGION 1..8: note = Synthetic
LQVRIVGG

SEQ ID NO: 278 - protein, 8 aa, synthetic construct
  REGION 1..8: note = Synthetic
PLGRIVGG

SEQ ID NO: 279 - protein, 8 aa, synthetic construct
  REGION 1..8: note = Synthetic
IEGRTVGG

SEQ ID NO: 280 - protein, 8 aa, synthetic construct
  REGION 1..8: note = Synthetic
LTPRSLLV

SEQ ID NO: 281 - protein, 8 aa, synthetic construct
  REGION 1..8: note = Synthetic
LGPVSGVP

SEQ ID NO: 282 - protein, 8 aa, synthetic construct
  REGION 1..8: note = Synthetic
VAGDSLEE

SEQ ID NO: 283 - protein, 8 aa, synthetic construct
  REGION 1..8: note = Synthetic
GPAGLGGA

SEQ ID NO: 284 - protein, 8 aa, synthetic construct
  REGION 1..8: note = Synthetic
GPAGLRGA

SEQ ID NO: 285 - protein, 8 aa, synthetic construct
  REGION 1..8: note = Synthetic
APLGLRLR

SEQ ID NO: 286 - protein, 8 aa, synthetic construct
  REGION 1..8: note = Synthetic
PALPLVAQ

SEQ ID NO: 287 - protein, 7 aa, synthetic construct
  REGION 1..7: note = Synthetic
ENLYFQG

SEQ ID NO: 288 - protein, 8 aa, synthetic construct
  REGION 1..8: note = Synthetic
DDDKIVGG

SEQ ID NO: 289 - protein, 12 aa, synthetic construct
  REGION 1..12: note = Synthetic
GSTSESPSGTAP

SEQ ID NO: 290 - protein, 12 aa, synthetic construct
  REGION 1..12: note = Synthetic
GTSTPESGSASP

SEQ ID NO: 291 - protein, 12 aa, synthetic construct
  REGION 1..12: note = Synthetic
GTSPSGESSTAP

SEQ ID NO: 292 - protein, 12 aa, synthetic construct
  REGION 1..12: note = Synthetic
GSTSSTAESPGP

SEQ ID NO: 293 - protein, 24 aa, synthetic construct
  REGION 1..24: note = Synthetic
GSPAGSPTSTEEGTSESATPESGP

SEQ ID NO: 294 - protein, 36 aa, synthetic construct
  REGION 1..36: note = Synthetic
GSPAGSPTSTEEGTSESATPESGPGTSTEPSEGSAP

SEQ ID NO: 295 - protein, 48 aa, synthetic construct
  REGION 1..48: note = Synthetic
GSPAGSPTSTEEGTSESATPESGPGTSTEPSEGSAPGSPAGSPTSTEE

SEQ ID NO: 296 - protein, 60 aa, synthetic construct
  REGION 1..60: note = Synthetic
GSPAGSPTSTEEGTSESATPESGPGTSTEPSEGSAPGSPAGSPTSTEEGTSTEPSEGSAP

SEQ ID NO: 297 - protein, 108 aa, synthetic construct
  REGION 1..108: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETP

SEQ ID NO: 298 - protein, 216 aa, synthetic construct
  REGION 1..216: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAP

SEQ ID NO: 299 - protein, 432 aa, synthetic construct
  REGION 1..432: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAP

SEQ ID NO: 300 - protein, 864 aa, synthetic construct; identical to SEQ ID NO: 213
  REGION 1..864: note = Synthetic

SEQ ID NO: 301 - protein, 576 aa, synthetic construct; identical to SEQ ID NO: 209
  REGION 1..576: note = Synthetic

SEQ ID NO: 302 - protein, 576 aa, synthetic construct; identical to SEQ ID NO: 210
  REGION 1..576: note = Synthetic

SEQ ID NO: 303 - protein, 540 aa, synthetic construct; identical to SEQ ID NO: 208
  REGION 1..540: note = Synthetic

SEQ ID NO: 304 - protein, 504 aa, synthetic construct
  REGION 1..504: note = Synthetic
GASPGTSSTGSPGSSPSASTGTGPGSSPSASTGTGPGTPGSGTASSSPGSSTPSGATGSPGSNPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSNPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSP

SEQ ID NO: 305 - protein, 864 aa, synthetic construct; identical to SEQ ID NO: 213
  REGION 1..864: note = Synthetic

SEQ ID NO: 306 - protein, 875 aa, synthetic construct; identical to SEQ ID NO: 214
  REGION 1..875: note = Synthetic
  VAR_SEQ 430..432: note = Xaa is any amino acid
  VAR_SEQ 443..446: note = Xaa is any amino acid

SEQ ID NO: 307 - protein, 868 aa, synthetic construct
  REGION 1..868: note = Synthetic
GGSPGASPGTSSTGSPGSSPSASTGTGPGSSPSASTGTGPGTPGSGTASSSPGSSTPSGATGSPGSNPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSNPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 308 - protein, 875 aa, synthetic construct; identical to SEQ ID NO: 216
  REGION 1..875: note = Synthetic

SEQ ID NO: 309 - protein, 1318 aa, synthetic construct; identical to SEQ ID NO: 219
  REGION 1..1318: note = Synthetic

SEQ ID NO: 310 - protein, 924 aa, synthetic construct; identical to SEQ ID NO: 218
  REGION 1..924: note = Synthetic

SEQ ID NO: 311 - protein, 913 aa, synthetic construct; identical to SEQ ID NO: 217
  REGION 1..913: note = Synthetic

SEQ ID NO: 312 - protein, 864 aa, synthetic construct; identical to SEQ ID NO: 220
  REGION 1..864: note = Synthetic

SEQ ID NO: 313 - protein, 84 aa, synthetic construct
  REGION 1..84: note = Synthetic
ASPAAPAPASPAAPAPSAPAAAPASPAPAAPSAPAPAAPSAASPAAPSAPPAAASPAAPSAPPAASAAAPAAASAAASAPSAAA

SEQ ID NO: 314 - protein, 288 aa, synthetic construct
  REGION 1..288: note = Synthetic
GSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEGGSGGEG

SEQ ID NO: 315 - protein, 288 aa, synthetic construct
  REGION 1..288: note = Synthetic
GEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEGGEGEGGGEG

SEQ ID NO: 316 - protein, 288 aa, synthetic construct
  REGION 1..288: note = Synthetic
SSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSESSSESSESSSSES

SEQ ID NO: 317 - protein, 288 aa, synthetic construct
  REGION 1..288: note = Synthetic
GEGSGEGSEGEGSEGSGEGEGSEGSGEGEGGSEGSEGEGGSEGSEGEGGSEGSEGEGSGEGSEGEGGSEGSEGEGSGEGSEGEGSEGGSEGEGGSEGSEGEGSGEGSEGEGGEGGSEGEGSEGSGEGEGSGEGSEGEGSEGSGEGEGSGEGSEGEGSEGSGEGEGSEGSGEGEGGSEGSEGEGSEGSGEGEGGEGSGEGEGSGEGSEGEGGGEGSEGEGSGEGGEGEGSEGGSEGEGGSEGGEGEGSEGSGEGEGSEGGSEGEGSEGGSEGEGSEGSGEGEGSEGSGE

SEQ ID NO: 318 - protein, 576 aa, synthetic construct
  REGION 1..576: note = Synthetic
GGKPGEGGKPEGGGGKPGGKPEGEGEGKPGGKPEGGGKPGGGEGGKPEGGKPEGEGKPGGGEGKPGGKPEGGGGKPEGEGKPGGGGGKPGGKPEGEGKPGGGEGGKPEGKPGEGGEGKPGGKPEGGGEGKPGGGKPGEGGKPGEGKPGGGEGGKPEGGKPEGEGKPGGGEGKPGGKPGEGGKPEGGGEGKPGGKPGEGGEGKPGGGKPEGEGKPGGGKPGGGEGGKPEGEGKPGGKPEGGGEGKPGGKPEGGGKPEGGGEGKPGGGKPGEGGKPGEGEGKPGGKPEGEGKPGGEGGGKPEGKPGGGEGGKPEGGKPGEGGKPEGGKPGEGGEGKPGGGKPGEGGKPEGGGKPEGEGKPGGGGKPGEGGKPEGGKPEGGGEGKPGGGKPEGEGKPGGGEGKPGGKPEGGGGKPGEGGKPEGGKPGGEGGGKPEGEGKPGGKPGEGGGGKPGGKPEGEGKPGEGGEGKPGGKPEGGGEGKPGGKPEGGGEGKPGGGKPGEGGKPEGGGKPGEGGKPGEGGKPEGEGKPGGGEGKPGGKPGEGGKPEGGGEGKPGGKPGGEGGGKPEGGKPGEGGKPEG

SEQ ID NO: 319 - protein, 558 aa, synthetic construct
  REGION 1..558: note = Synthetic
GEGKPGGKPGSGGGKPGEGGKPGSGEGKPGGKPGSGGSGKPGGKPGEGGKPEGGSGGKPGGGGKPGGKPGGEGSGKPGGKPEGGGKPEGGSGGKPGGKPEGGSGGKPGGKPGSGEGGKPGGGKPGGEGKPGSGKPGGEGSGKPGGKPEGGSGGKPGGKPEGGSGGKPGGSGKPGGKPGEGGKPEGGSGGKPGGSGKPGGKPEGGGSGKPGGKPGEGGKPGSGEGGKPGGGKPGGEGKPGSGKPGGEGSGKPGGKPGSGGEGKPGGKPEGGSGGKPGGGKPGGEGKPGSGGKPGEGGKPGSGGGKPGGKPGGEGEGKPGGKPGEGGKPGGEGSGKPGGGGKPGGKPGGEGGKPEGSGKPGGGSGKPGGKPEGGGGKPEGSGKPGGGGKPEGSGKPGGGKPEGGSGGKPGGSGKPGGKPGEGGGKPEGSGKPGGGSGKPGGKPEGGGKPEGGSGGKPGGKPEGGSGGKPGGKPGGEGSGKPGGKPGSGEGGKPGGKPGEGSGGKPGGKPEGGSGGKPGGSGKPGGKPEGGGSGKPGGKPGEGGKPGGEGSGKPGGSGKPG

SEQ ID NO: 320 - protein, 577 aa, synthetic construct
  REGION 1..577: note = Synthetic
GGSGKPGKPGGSGSGKPGSGKPGGGSGKPGSGKPGGGSGKPGSGKPGGGSGKPGSGKPGGGGKPGSGSGKPGGGKPGGSGGKPGGGSGKPGKPGSGGSGKPGSGKPGGGSGGKPGKPGSGGSGGKPGKPGSGGGSGKPGKPGSGGSGGKPGKPGSGGSGGKPGKPGSGGSGKPGSGKPGGGSGKPGSGKPGSGGSGKPGKPGSGGSGKPGSGKPGSGSGKPGSGKPGGGSGKPGSGKPGSGGSGKPGKPGSGGGKPGSGSGKPGGGKPGSGSGKPGGGKPGGSGGKPGGSGGKPGKPGSGGGSGKPGKPGSGGGSGKPGKPGGSGSGKPGSGKPGGGSGKPGSGKPGSGGSGKPGKPGSGGSGGKPGKPGSGGGKPGSGSGKPGGGKPGSGSGKPGGGKPGSGSGKPGGGKPGSGSGKPGGSGKPGSGKPGGGSGGKPGKPGSGGSGKPGSGKPGSGGSGKPGKPGGSGSGKPGSGKPGGGSGKPGSGKPGGGSGKPGSGKPGGGSGKPGSGKPGGGGKPGSGSGKPGGSGGKPGKPGSGGSGGKPGKPGSGGSGKPGSGKPGGGSGGKPGKPGSGG

SEQ ID NO: 321 - protein, 576 aa, synthetic construct
  REGION 1..576: note = Synthetic
GEGSGEGSEGEGSEGSGEGEGSEGSGEGEGGSEGSEGEGSEGSGEGEGGEGSGEGEGSGEGSEGEGGGEGSEGEGSGEGGEGEGSEGGSEGEGGSEGGEGEGSEGSGEGEGSEGGSEGEGSEGGSEGEGSEGSGEGEGSEGSGEGEGSEGSGEGEGSEGSGEGEGSEGGSEGEGGSEGSEGEGSGEGSEGEGGSEGSEGEGGGEGSEGEGSGEGSEGEGGSEGSEGEGGSEGSEGEGGEGSGEGEGSEGSGEGEGSGEGSEGEGSEGSGEGEGSEGSGEGEGGSEGSEGEGSGEGSEGEGSEGSGEGEGSEGSGEGEGGSEGSEGEGGSEGSEGEGGSEGSEGEGGEGSGEGEGSEGSGEGEGSGEGSEGEGSEGSGEGEGSEGSGEGEGGSEGSEGEGSEGSGEGEGGEGSGEGEGSGEGSEGEGGGEGSEGEGSEGSGEGEGSEGSGEGEGSEGGSEGEGGSEGSEGEGSEGGSEGEGSEGGSEGEGSEGSGEGEGSEGSGEGEGSGEGSEGEGGSEGGEGEGSEGGSEGEGSEGGSEGEGGEGSGEGEGGGEGSEGEGSEGSGEGEGSGEGSE

SEQ ID NO: 322 - protein, 576 aa, synthetic construct; identical to SEQ ID NO: 209
  REGION 1..576: note = Synthetic

SEQ ID NO: 323 - protein, 577 aa, synthetic construct
  REGION 1..577: note = Synthetic
AGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 324 - protein, 540 aa, synthetic construct; identical to SEQ ID NO: 208
  REGION 1..540: note = Synthetic

SEQ ID NO: 325 - protein, 504 aa, synthetic construct; identical to SEQ ID NO: 304
  REGION 1..504: note = Synthetic

SEQ ID NO: 326 - protein, 816 aa, synthetic construct
  REGION 1..816: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAP

SEQ ID NO: 327 - protein, 875 aa, synthetic construct; identical to SEQ ID NO: 214
  REGION 1..875: note = Synthetic
  VAR_SEQ 430..432: note = Xaa is any amino acid
  VAR_SEQ 443..446: note = Xaa is any amino acid

SEQ ID NO: 328 - protein, 868 aa, synthetic construct; identical to SEQ ID NO: 307
  REGION 1..868: note = Synthetic

SEQ ID NO: 329 - protein, 875 aa, synthetic construct; identical to SEQ ID NO: 216
  REGION 1..875: note = Synthetic

SEQ ID NO: 330 - protein, 1318 aa, synthetic construct; identical to SEQ ID NO: 219
  REGION 1..1318: note = Synthetic

SEQ ID NO: 331 - protein, 1018 aa, synthetic construct
  REGION 1..1018: note = Synthetic
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEGGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 332 - protein, 1029 aa, synthetic construct
  REGION 1..1029: note = Synthetic
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEGGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 333 - protein, 1042 aa, synthetic construct
  REGION 1..1042: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 334 - protein, 1053 aa, synthetic construct
  REGION 1..1053: note = Synthetic
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAPMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 335 - protein, 29 aa, synthetic construct
  REGION 1..29: note = Synthetic
GSAPGSAGGYAELRMGGAIATSGSETPGT

SEQ ID NO: 336 - protein, 29 aa, synthetic construct
  REGION 1..29: note = Synthetic
GSAPGTGGGYAPLRMGGGAATSGSETPGT

SEQ ID NO: 337 - protein, 29 aa, synthetic construct
  REGION 1..29: note = Synthetic
GSAPGAEGGYAALRMGGEIATSGSETPGT

SEQ ID NO: 338 - protein, 29 aa, synthetic construct
  REGION 1..29: note = Synthetic
GSAPGGPGGYALLRMGGPAATSGSETPGT

SEQ ID NO: 339 - protein, 29 aa, synthetic construct
  REGION 1..29: note = Synthetic
GSAPGEAGGYAFLRMGGSIATSGSETPGT

SEQ ID NO: 340 - protein, 29 aa, synthetic construct
  REGION 1..29: note = Synthetic
GSAPGPGGGYASLRMGGTAATSGSETPGT

SEQ ID NO: 341 - protein, 29 aa, synthetic construct
  REGION 1..29: note = Synthetic
GSAPGSEGGYATLRMGGAIATSGSETPGT

SEQ ID NO: 342 - protein, 29 aa, synthetic construct
  REGION 1..29: note = Synthetic
GSAPGTPGGYANLRMGGGAATSGSETPGT

SEQ ID NO: 343 - protein, 29 aa, synthetic construct
  REGION 1..29: note = Synthetic
GSAPGASGGYAHLRMGGEIATSGSETPGT

SEQ ID NO: 344 - protein, 29 aa, synthetic construct
  REGION 1..29: note = Synthetic
GSAPGGTGGYGELRMGGPAATSGSETPGT

SEQ ID NO: 345 - protein, 29 aa, synthetic construct
  REGION 1..29: note = Synthetic
GSAPGEAGGYPELRMGGSIATSGSETPGT

SEQ ID NO: 346 - protein, 29 aa, synthetic construct
  REGION 1..29: note = Synthetic
GSAPGPGGGYVELRMGGTAATSGSETPGT

SEQ ID NO: 347 - protein, 29 aa, synthetic construct
  REGION 1..29: note = Synthetic
GSAPGSEGGYLELRMGGAIATSGSETPGT

SEQ ID NO: 348 - protein, 29 aa, synthetic construct
  REGION 1..29: note = Synthetic
GSAPGTPGGYSELRMGGGAATSGSETPGT

SEQ ID NO: 349 - protein, 29 aa, synthetic construct
  REGION 1..29: note = Synthetic
GSAPGASGGYTELRMGGEIATSGSETPGT

SEQ ID NO: 350 - protein, 29 aa, synthetic construct
  REGION 1..29: note = Synthetic
GSAPGGTGGYQELRMGGPAATSGSETPGT

SEQ ID NO: 351 - protein, 29 aa, synthetic construct
  REGION 1..29: note = Synthetic
GSAPGEAGGYEELRMGGSIATSGSETPGT

SEQ ID NO: 352 - protein, 29 aa, synthetic construct
  REGION 1..29: note = Synthetic
GSAPGPGIGPAELRMGGTAATSGSETPGT

SEQ ID NO: 353 - protein, 29 aa, synthetic construct
  REGION 1..29: note = Synthetic
GSAPGSEIGAAELRMGGAIATSGSETPGT

SEQ ID NO: 354 - protein, 29 aa, synthetic construct
  REGION 1..29: note = Synthetic
GSAPGTPIGSAELRMGGGAATSGSETPGT

SEQ ID NO: 355 - protein, 29 aa, synthetic construct
  REGION 1..29: note = Synthetic
GSAPGASIGTAELRMGGEIATSGSETPGT

SEQ ID NO: 356 - protein, 29 aa, synthetic construct
  REGION 1..29: note = Synthetic
GSAPGGTIGNAELRMGGPAATSGSETPGT

SEQ ID NO: 357 - protein, 29 aa, synthetic construct
  REGION 1..29: note = Synthetic
GSAPGEAIGQAELRMGGSIATSGSETPGT

SEQ ID NO: 358 - protein, 29 aa, synthetic construct
  REGION 1..29: note = Synthetic
GSAPGPGGPYAELRMGGTAATSGSETPGT

SEQ ID NO: 359 - protein, 29 aa, synthetic construct
  REGION 1..29: note = Synthetic
GSAPGSEGAYAELRMGGAIATSGSETPGT

SEQ ID NO: 360 - protein, 29 aa, synthetic construct
  REGION 1..29: note = Synthetic
GSAPGTPGVYAELRMGGGAATSGSETPGT

SEQ ID NO: 361 - protein, 29 aa, synthetic construct
  REGION 1..29: note = Synthetic
GSAPGASGLYAELRMGGEIATSGSETPGT

SEQ ID NO: 362 - protein, 29 aa, synthetic construct
  REGION 1..29: note = Synthetic
GSAPGGTGIYAELRMGGPAATSGSETPGT

SEQ ID NO: 363 - protein, 29 aa, synthetic construct
  REGION 1..29: note = Synthetic
GSAPGEAGFYAELRMGGSIATSGSETPGT

SEQ ID NO: 364 - protein, 29 aa, synthetic construct
  REGION 1..29: note = Synthetic
GSAPGPGGYYAELRMGGTAATSGSETPGT

SEQ ID NO: 365 - protein, 29 aa, synthetic construct
  REGION 1..29: note = Synthetic
GSAPGSEGSYAELRMGGAIATSGSETPGT

SEQ ID NO: 366 - protein, 29 aa, synthetic construct
  REGION 1..29: note = Synthetic
GSAPGTPGNYAELRMGGGAATSGSETPGT

SEQ ID NO: 367 - protein, 29 aa, synthetic construct
  REGION 1..29: note = Synthetic
GSAPGASGEYAELRMGGEIATSGSETPGT

SEQ ID NO: 368 - protein, 29 aa, synthetic construct
  REGION 1..29: note = Synthetic
GSAPGGTGHYAELRMGGPAATSGSETPGT

SEQ ID NO: 369 - protein, 29 aa, synthetic construct
  REGION 1..29: note = Synthetic
GSAPGEAGGYAEARMGGSIATSGSETPGT

SEQ ID NO: 370 - protein, 29 aa, synthetic construct
  REGION 1..29: note = Synthetic
GSAPGPGGGYAEVRMGGTAATSGSETPGT

SEQ ID NO: 371 - protein, 29 aa, synthetic construct
  REGION 1..29: note = Synthetic
GSAPGSEGGYAEIRMGGAIATSGSETPGT

SEQ ID NO: 372 - protein, 29 aa, synthetic construct
  REGION 1..29: note = Synthetic
GSAPGTPGGYAEFRMGGGAATSGSETPGT

SEQ ID NO: 373 - protein, 29 aa, synthetic construct
  REGION 1..29: note = Synthetic
GSAPGASGGYAEYRMGGEIATSGSETPGT

SEQ ID NO: 374 - protein, 29 aa, synthetic construct
  REGION 1..29: note = Synthetic
GSAPGGTGGYAESRMGGPAATSGSETPGT

SEQ ID NO: 375 - protein, 29 aa, synthetic construct
  REGION 1..29: note = Synthetic
GSAPGEAGGYAETRMGGSIATSGSETPGT

SEQ ID NO: 376 - protein, 29 aa, synthetic construct
  REGION 1..29: note = Synthetic
GSAPGPGGGYAELAMGGTRATSGSETPGT

SEQ ID NO: 377 - protein, 29 aa, synthetic construct
  REGION 1..29: note = Synthetic
GSAPGSEGGYAELVMGGARATSGSETPGT

SEQ ID NO: 378 - protein, 29 aa, synthetic construct
  REGION 1..29: note = Synthetic
GSAPGTPGGYAELLMGGGRATSGSETPGT

SEQ ID NO: 379 - protein, 29 aa, synthetic construct
  REGION 1..29: note = Synthetic
GSAPGASGGYAELIMGGERATSGSETPGT

SEQ ID NO: 380 - protein, 29 aa, synthetic construct
  REGION 1..29: note = Synthetic
GSAPGGTGGYAELWMGGPRATSGSETPGT

SEQ ID NO: 381 - protein, 29 aa, synthetic construct
  REGION 1..29: note = Synthetic
GSAPGEAGGYAELSMGGSRATSGSETPGT

SEQ ID NO: 382 - protein, 29 aa, synthetic construct
  REGION 1..29: note = Synthetic
GSAPGPGGGYAELTMGGTRATSGSETPGT

SEQ ID NO: 383 - protein, 29 aa, synthetic construct
  REGION 1..29: note = Synthetic
GSAPGSEGGYAELQMGGARATSGSETPGT

SEQ ID NO: 384 - protein, 29 aa, synthetic construct
  REGION 1..29: note = Synthetic
GSAPGTPGGYAELNMGGGRATSGSETPGT

SEQ ID NO: 385 - protein, 29 aa, synthetic construct
  REGION 1..29: note = Synthetic
GSAPGASGGYAELEMGGERATSGSETPGT

SEQ ID NO: 386 - protein, 29 aa, synthetic construct
  REGION 1..29: note = Synthetic
GSAPGGTGGYAELRPGGPIATSGSETPGT

SEQ ID NO: 387 - protein, 29 aa, synthetic construct
  REGION 1..29: note = Synthetic
GSAPGEAGGYAELRAGGSAATSGSETPGT

SEQ ID NO: 388 - protein, 29 aa, synthetic construct
  REGION 1..29: note = Synthetic
GSAPGPGGGYAELRLGGTIATSGSETPGT

SEQ ID NO: 389 - protein, 29 aa, synthetic construct
  REGION 1..29: note = Synthetic
GSAPGSEGGYAELRIGGAAATSGSETPGT

SEQ ID NO: 390 - protein, 29 aa, synthetic construct
  REGION 1..29: note = Synthetic
GSAPGTPGGYAELRSGGGIATSGSETPGT

SEQ ID NO: 391 - protein, 29 aa, synthetic construct
  REGION 1..29: note = Synthetic
GSAPGASGGYAELRNGGEAATSGSETPGT

SEQ ID NO: 392 - protein, 29 aa, synthetic construct
  REGION 1..29: note = Synthetic
GSAPGGTGGYAELRQGGPIATSGSETPGT

SEQ ID NO: 393 - protein, 29 aa, synthetic construct
  REGION 1..29: note = Synthetic
GSAPGEAGGYAELRDGGSAATSGSETPGT

SEQ ID NO: 394 - protein, 29 aa, synthetic construct
  REGION 1..29: note = Synthetic
GSAPGPGGGYAELREGGTIATSGSETPGT

SEQ ID NO: 395 - protein, 29 aa, synthetic construct
  REGION 1..29: note = Synthetic
GSAPGSEGGYAELRHGGAAATSGSETPGT

SEQ ID NO: 396 - protein, 29 aa, synthetic construct
  REGION 1..29: note = Synthetic
GSAPGTPGGYAELRMPGGIATSGSETPGT

SEQ ID NO: 397 - protein, 29 aa, synthetic construct
  REGION 1..29: note = Synthetic
GSAPGASGGYAELRMAGEAATSGSETPGT

SEQ ID NO: 398 - protein, 29 aa, synthetic construct
  REGION 1..29: note = Synthetic
GSAPGGTGGYAELRMVGPIATSGSETPGT

SEQ ID NO: 399 - protein, 29 aa, synthetic construct
  REGION 1..29: note = Synthetic
GSAPGEAGGYAELRMLGSAATSGSETPGT

SEQ ID NO: 400 - protein, 29 aa, synthetic construct
  REGION 1..29: note = Synthetic
GSAPGPGGGYAELRMIGTIATSGSETPGT

SEQ ID NO: 401 - protein, 29 aa, synthetic construct
  REGION 1..29: note = Synthetic
GSAPGSEGGYAELRMYGAIATSGSETPGT

SEQ ID NO: 402 - protein, 29 aa, synthetic construct
  REGION 1..29: note = Synthetic
GSAPGTPGGYAELRMSGGAATSGSETPGT

SEQ ID NO: 403 - protein, 29 aa, synthetic construct
  REGION 1..29: note = Synthetic
GSAPGASGGYAELRMNGEIATSGSETPGT

SEQ ID NO: 404 - protein, 29 aa, synthetic construct
  REGION 1..29: note = Synthetic
GSAPGGTGGYAELRMQGPAATSGSETPGT

SEQ ID NO: 405 - protein, 29 aa, synthetic construct
  REGION 1..29: note = Synthetic
GSAPGANHTPAGLTGPGARATSGSETPGT

SEQ ID NO: 406 - protein, 29 aa, synthetic construct
  REGION 1..29: note = Synthetic
GSAPGANTAPEGLTGPSTRATSGSETPGT

SEQ ID NO: 407 - protein, 29 aa, synthetic construct
  REGION 1..29: note = Synthetic
GSAPGTGAPPGGLTGPGTRATSGSETPGT

SEQ ID NO: 408 - protein, 29 aa, synthetic construct
  REGION 1..29: note = Synthetic
GSAPGANHEPSGLTEGSPRATSGSETPGT

SEQ ID NO: 409 - protein, 29 aa, synthetic construct
  REGION 1..29: note = Synthetic
GSAPGANTEPPELGAGTERATSGSETPGT

SEQ ID NO: 410 - protein, 29 aa, synthetic construct
  REGION 1..29: note = Synthetic
GSAPGASGPPPGLTGPPGRATSGSETPGT

SEQ ID NO: 411 - protein, 29 aa, synthetic construct
  REGION 1..29: note = Synthetic
GSAPGASGTPAPLGGEPGRATSGSETPGT

SEQ ID NO: 412 - protein, 29 aa, synthetic construct
  REGION 1..29: note = Synthetic
GSAPGPAGPPEGLETEAGRATSGSETPGT

SEQ ID NO: 413 - protein, 29 aa, synthetic construct
  REGION 1..29: note = Synthetic
GSAPGPTSGQGGLTGPESRATSGSETPGT

SEQ ID NO: 414 - protein, 29 aa, synthetic construct
  REGION 1..29: note = Synthetic
GSAPGSAGGAANLVRGGAIATSGSETPGT

SEQ ID NO: 415 - protein, 29 aa, synthetic construct
  REGION 1..29: note = Synthetic
GSAPGTGGGAAPLVRGGGAATSGSETPGT

SEQ ID NO: 416 - protein, 29 aa, synthetic construct
  REGION 1..29: note = Synthetic
GSAPGAEGGAAALVRGGEIATSGSETPGT

SEQ ID NO: 417 - protein, 29 aa, synthetic construct
  REGION 1..29: note = Synthetic
GSAPGGPGGAALLVRGGPAATSGSETPGT

SEQ ID NO: 418 - protein, 29 aa, synthetic construct
  REGION 1..29: note = Synthetic
GSAPGEAGGAAFLVRGGSIATSGSETPGT

SEQ ID NO: 419 - protein, 29 aa, synthetic construct
  REGION 1..29: note = Synthetic
GSAPGPGGGAASLVRGGTAATSGSETPGT

SEQ ID NO: 420 - protein, 29 aa, synthetic construct
  REGION 1..29: note = Synthetic
GSAPGSEGGAATLVRGGAIATSGSETPGT

SEQ ID NO: 421 - protein, 29 aa, synthetic construct
  REGION 1..29: note = Synthetic
GSAPGTPGGAAGLVRGGGAATSGSETPGT

SEQ ID NO: 422 - protein, 29 aa, synthetic construct
  REGION 1..29: note = Synthetic
GSAPGASGGAADLVRGGEIATSGSETPGT

SEQ ID NO: 423 - protein, 29 aa, synthetic construct
  REGION 1..29: note = Synthetic
GSAPGGTGGAGNLVRGGPAATSGSETPGT

SEQ ID NO: 424 - protein, 29 aa, synthetic construct
  REGION 1..29: note = Synthetic
GSAPGEAGGAPNLVRGGSIATSGSETPGT

SEQ ID NO: 425 - protein, 29 aa, synthetic construct
  REGION 1..29: note = Synthetic
GSAPGPGGGAVNLVRGGTAATSGSETPGT

SEQ ID NO: 426 - protein, 29 aa, synthetic construct
  REGION 1..29: note = Synthetic
GSAPGSEGGALNLVRGGAIATSGSETPGT

SEQ ID NO: 427 - protein, 29 aa, synthetic construct
  REGION 1..29: note = Synthetic
GSAPGTPGGASNLVRGGGAATSGSETPGT

SEQ ID NO: 428 - protein, 29 aa, synthetic construct
  REGION 1..29: note = Synthetic
GSAPGASGGATNLVRGGEIATSGSETPGT

SEQ ID NO: 429 - protein, 29 aa, synthetic construct
  REGION 1..29: note = Synthetic
GSAPGGTGGAQNLVRGGPAATSGSETPGT

SEQ ID NO: 430 - protein, 29 aa, synthetic construct
  REGION 1..29: note = Synthetic
GSAPGEAGGAENLVRGGSIATSGSETPGT

SEQ ID NO: 431 - protein, 30 aa, synthetic construct
  REGION 1..30: note = Synthetic
GSAPEAGRSANHEPLGLVATATSGSETPGT

SEQ ID NO: 432 - protein, 30 aa, synthetic construct
  REGION 1..30: note = Synthetic
GSAPASGRSTNAGPSGLAGPATSGSETPGT

SEQ ID NO: 433 - protein, 30 aa, synthetic construct
  REGION 1..30: note = Synthetic
GSAPASGRSTNAGPQGLAGQATSGSETPGT

SEQ ID NO: 434 - protein, 30 aa, synthetic construct
  REGION 1..30: note = Synthetic
GSAPASGRSTNAGPPGLTGPATSGSETPGT

SEQ ID NO: 435 - protein, 30 aa, synthetic construct
  REGION 1..30: note = Synthetic
GSAPASSRGTNAGPAGLTGPATSGSETPGT

SEQ ID NO: 436 - protein, 30 aa, synthetic construct
  REGION 1..30: note = Synthetic
GSAPASSRTTNTGPSTLTGPATSGSETPGT

SEQ ID NO: 437 - protein, 30 aa, synthetic construct
  REGION 1..30: note = Synthetic
GSAPAAGRSDNGTPLELVAPATSGSETPGT

SEQ ID NO: 438 - protein, 30 aa, synthetic construct; identical to SEQ ID NO: 431
  REGION 1..30: note = Synthetic

SEQ ID NO: 439 - protein, 30 aa, synthetic construct
  REGION 1..30: note = Synthetic
GSAPASGRGTNAGPAGLTGPATSGSETPGT

SEQ ID NO: 440 - protein, 30 aa, synthetic construct
  REGION 1..30: note = Synthetic
GSAPLFGRNDNHEPLELGGGATSGSETPGT

SEQ ID NO: 441 - protein, 30 aa, synthetic construct
  REGION 1..30: note = Synthetic
GSAPTAGRSDNLEPLGLVFGATSGSETPGT

SEQ ID NO: 442 - protein, 30 aa, synthetic construct
  REGION 1..30: note = Synthetic
GSAPLDGRSDNFHPPELVAGATSGSETPGT

SEQ ID NO: 443 - protein, 30 aa, synthetic construct
  REGION 1..30: note = Synthetic
GSAPLEGRSDNEEPENLVAGATSGSETPGT

SEQ ID NO: 444 - protein, 30 aa, synthetic construct
  REGION 1..30: note = Synthetic
GSAPLKGRSDNNAPLALVAGATSGSETPGT

SEQ ID NO: 445 - protein, 30 aa, synthetic construct
  REGION 1..30: note = Synthetic
GSAPVYSRGTNAGPHGLTGRATSGSETPGT

SEQ ID NO: 446 - protein, 30 aa, synthetic construct
  REGION 1..30: note = Synthetic
GSAPANSRGTNKGFAGLIGPATSGSETPGT

SEQ ID NO: 447 - protein, 30 aa, synthetic construct
  REGION 1..30: note = Synthetic
GSAPASSRLTNEAPAGLTIPATSGSETPGT

SEQ ID NO: 448 - protein, 30 aa, synthetic construct
  REGION 1..30: note = Synthetic
GSAPDQSRGTNAGPEGLTDPATSGSETPGT

SEQ ID NO: 449 - protein, 30 aa, synthetic construct
  REGION 1..30: note = Synthetic
GSAPESSRGTNIGQGGLTGPATSGSETPGT

SEQ ID NO: 450 - protein, 30 aa, synthetic construct
  REGION 1..30: note = Synthetic
GSAPSSSRGTNQDPAGLTIPATSGSETPGT

SEQ ID NO: 451 - protein, 30 aa, synthetic construct
  REGION 1..30: note = Synthetic
GSAPASSRGQNHSPMGLTGPATSGSETPGT

SEQ ID NO: 452 - protein, 30 aa, synthetic construct
  REGION 1..30: note = Synthetic
GSAPAYSRGPNAGPAGLEGRATSGSETPGT

SEQ ID NO: 453 - protein, 30 aa, synthetic construct
  REGION 1..30: note = Synthetic
GSAPASERGNNAGPANLTGFATSGSETPGT

SEQ ID NO: 454 - protein, 30 aa, synthetic construct
  REGION 1..30: note = Synthetic
GSAPASHRGTNPKPAILTGPATSGSETPGT

SEQ ID NO: 455 - protein, 30 aa, synthetic construct
  REGION 1..30: note = Synthetic
GSAPMSSRRTNANPAQLTGPATSGSETPGT

SEQ ID NO: 456 - protein, 30 aa, synthetic construct
  REGION 1..30: note = Synthetic
GSAPGAGRTDNHEPLELGAAATSGSETPGT

SEQ ID NO: 457 - protein, 30 aa, synthetic construct
  REGION 1..30: note = Synthetic
GSAPLAGRSENTAPLELTAGATSGSETPGT

SEQ ID NO: 458 - protein, 30 aa, synthetic construct
  REGION 1..30: note = Synthetic
GSAPLEGRPDNHEPLALVASATSGSETPGT

SEQ ID NO: 459 - protein, 30 aa, synthetic construct
  REGION 1..30: note = Synthetic
GSAPLSGRSDNEEPLALPAGATSGSETPGT

SEQ ID NO: 460 - protein, 30 aa, synthetic construct
  REGION 1..30: note = Synthetic
GSAPEAGRTDNHEPLELSAPATSGSETPGT

SEQ ID NO: 461 - protein, 30 aa, synthetic construct
  REGION 1..30: note = Synthetic
GSAPEGGRSDNHGPLELVSGATSGSETPGT

SEQ ID NO: 462 - protein, 30 aa, synthetic construct
  REGION 1..30: note = Synthetic
GSAPLSGRSDNEAPLELEAGATSGSETPGT

SEQ ID NO: 463 - protein, 30 aa, synthetic construct
  REGION 1..30: note = Synthetic
GSAPLGGRADNHEPPELGAGATSGSETPGT

SEQ ID NO: 464 - protein, 30 aa, synthetic construct
  REGION 1..30: note = Synthetic
GSAPPPSRGTNAEPAGLTGEATSGSETPGT

SEQ ID NO: 465 - protein, 30 aa, synthetic construct
  REGION 1..30: note = Synthetic
GSAPASTRGENAGPAGLEAPATSGSETPGT

SEQ ID NO: 466 - protein, 30 aa, synthetic construct
  REGION 1..30: note = Synthetic
GSAPESSRGTNGAPEGLTGPATSGSETPGT

SEQ ID NO: 467 - protein, 30 aa, synthetic construct
  REGION 1..30: note = Synthetic
GSAPASSRATNESPAGLTGEATSGSETPGT

SEQ ID NO: 468 - protein, 30 aa, synthetic construct
  REGION 1..30: note = Synthetic
GSAPASSRGENPPPGGLTGPATSGSETPGT

SEQ ID NO: 469 - protein, 30 aa, synthetic construct
  REGION 1..30: note = Synthetic
GSAPAASRGTNTGPAELTGSATSGSETPGT

SEQ ID NO: 470 - protein, 30 aa, synthetic construct
  REGION 1..30: note = Synthetic
GSAPAGSRTTNAGPGGLEGPATSGSETPGT

SEQ ID NO: 471 - protein, 30 aa, synthetic construct
  REGION 1..30: note = Synthetic
GSAPAPSRGENAGPATLTGAATSGSETPGT

SEQ ID NO: 472 - protein, 30 aa, synthetic construct
  REGION 1..30: note = Synthetic
GSAPESGRAANTGPPTLTAPATSGSETPGT

SEQ ID NO: 473 - protein, 30 aa, synthetic construct
  REGION 1..30: note = Synthetic
GSAPNPGRAANEGPPGLPGSATSGSETPGT

SEQ ID NO: 474 - protein, 30 aa, synthetic construct
  REGION 1..30: note = Synthetic
GSAPESSRAANLTPPELTGPATSGSETPGT

SEQ ID NO: 475 - protein, 30 aa, synthetic construct
  REGION 1..30: note = Synthetic
GSAPASGRAANETPPGLTGAATSGSETPGT

SEQ ID NO: 476 - protein, 30 aa, synthetic construct
  REGION 1..30: note = Synthetic
GSAPNSGRGENLGAPGLTGTATSGSETPGT

SEQ ID NO: 477 - protein, 30 aa, synthetic construct
  REGION 1..30: note = Synthetic
GSAPTTGRAANLTPAGLTGPATSGSETPGT

SEQ ID NO: 478 - protein, 30 aa, synthetic construct
  REGION 1..30: note = Synthetic
GSAPEAGRSANHTPAGLTGPATSGSETPGT

SEQ ID NO: 479 - protein, 30 aa, synthetic construct
  REGION 1..30: note = Synthetic
GSAPESGRAANTTPAGLTGPATSGSETPGT

SEQ ID NO: 480 - protein, 33 aa, synthetic construct
  REGION 1..33: note = Synthetic
GSAPTTGRATEAANLTPAGLTGPATSGSETPGT

SEQ ID NO: 481 - protein, 33 aa, synthetic construct
  REGION 1..33: note = Synthetic
GSAPTTGRAEEAANLTPAGLTGPATSGSETPGT

SEQ ID NO: 482 - protein, 33 aa, synthetic construct
  REGION 1..33: note = Synthetic
GSAPTTGRAGEAANLTPAGLTGPATSGSETPGT

SEQ ID NO: 483 - protein, 33 aa, synthetic construct
  REGION 1..33: note = Synthetic
GSAPTTGRATEAANATPAGLTGPATSGSETPGT

SEQ ID NO: 484 - protein, 33 aa, synthetic construct
  REGION 1..33: note = Synthetic
GSAPTTGRAGEAEGATSAGATGPATSGSETPGT

SEQ ID NO: 485 - protein, 33 aa, synthetic construct
  REGION 1..33: note = Synthetic
GSAPTTGEAGEAANATSAGATGPATSGSETPGT

SEQ ID NO: 486 - protein, 33 aa, synthetic construct
  REGION 1..33: note = Synthetic
GSAPTTGEAGEAAGLTPAGLTGPATSGSETPGT

SEQ ID NO: 487 - protein, 33 aa, synthetic construct
  REGION 1..33: note = Synthetic
GSAPTTGAAGEAANATPAGLTGPATSGSETPGT

SEQ ID NO: 488 - protein, 33 aa, synthetic construct
  REGION 1..33: note = Synthetic
GSAPTTGRAGEAAGLTPAGLTGPATSGSETPGT

SEQ ID NO: 489 - protein, 33 aa, synthetic construct
  REGION 1..33: note = Synthetic
GSAPTTGRAGEAANATSAGATGPATSGSETPGT

SEQ ID NO: 490 - protein, 33 aa, synthetic construct
  REGION 1..33: note = Synthetic
GSAPTTGEAGEAAGATSAGATGPATSGSETPGT

SEQ ID NO: 491 - protein, 30 aa, synthetic construct
  REGION 1..30: note = Synthetic
GSAPESGRAANTEPPELGAGATSGSETPGT

SEQ ID NO: 492 - protein, 30 aa, synthetic construct
  REGION 1..30: note = Synthetic
GSAPESGRAANTAPEGLTGPATSGSETPGT

SEQ ID NO: 493 - protein, 30 aa, synthetic construct
  REGION 1..30: note = Synthetic
GSAPEPGRAANHEPSGLTEGATSGSETPGT

SEQ ID NO: 494 - protein, 33 aa, synthetic construct
  REGION 1..33: note = Synthetic
GSAPESGRAANHTGAPPGGLTGPATSGSETPGT

SEQ ID NO: 495 - protein, 33 aa, synthetic construct
  REGION 1..33: note = Synthetic
GSAPTTGRTGEGANATPGGLTGPATSGSETPGT

SEQ ID NO: 496 - protein, 33 aa, synthetic construct
  REGION 1..33: note = Synthetic
GSAPRTGRSGEAANETPEGLEGPATSGSETPGT

SEQ ID NO: 497 - protein, 33 aa, synthetic construct
  REGION 1..33: note = Synthetic
GSAPRTGRTGESANETPAGLGGPATSGSETPGT

SEQ ID NO: 498 - protein, 33 aa, synthetic construct
  REGION 1..33: note = Synthetic
GSAPSTGRTGEPANETPAGLSGPATSGSETPGT

SEQ ID NO: 499 - protein, 33 aa, synthetic construct
  REGION 1..33: note = Synthetic
GSAPTTGRAGEPANATPTGLSGPATSGSETPGT

SEQ ID NO: 500 - protein, 33 aa, synthetic construct
  REGION 1..33: note = Synthetic
GSAPRTGRPGEGANATPTGLPGPATSGSETPGT

SEQ ID NO: 501 - protein, 33 aa, synthetic construct
  REGION 1..33: note = Synthetic
GSAPRTGRGGEAANATPSGLGGPATSGSETPGT

SEQ ID NO: 502 - protein, 33 aa, synthetic construct
  REGION 1..33: note = Synthetic
GSAPSTGRSGESANATPGGLGGPATSGSETPGT

SEQ ID NO: 503 - protein, 33 aa, synthetic construct
  REGION 1..33: note = Synthetic
GSAPRTGRTGEEANATPAGLPGPATSGSETPGT

SEQ ID NO: 504 - protein, 33 aa, synthetic construct
  REGION 1..33: note = Synthetic
GSAPATGRPGEPANTTPEGLEGPATSGSETPGT

SEQ ID NO: 505 - protein, 33 aa, synthetic construct
  REGION 1..33: note = Synthetic
GSAPSTGRSGEPANATPGGLTGPATSGSETPGT

SEQ ID NO: 506 - protein, 33 aa, synthetic construct
  REGION 1..33: note = Synthetic
GSAPPTGRGGEGANTTPTGLPGPATSGSETPGT

SEQ ID NO: 507 - protein, 33 aa, synthetic construct
  REGION 1..33: note = Synthetic
GSAPPTGRSGEGANATPSGLTGPATSGSETPGT

SEQ ID NO: 508 - protein, 33 aa, synthetic construct
  REGION 1..33: note = Synthetic
GSAPTTGRASEGANSTPAPLTEPATSGSETPGT

SEQ ID NO: 509 - protein, 33 aa, synthetic construct
  REGION 1..33: note = Synthetic
GSAPTYGRAAEAANTTPAGLTAPATSGSETPGT

SEQ ID NO: 510 - protein, 33 aa, synthetic construct
  REGION 1..33: note = Synthetic
GSAPTTGRATEGANATPAELTEPATSGSETPGT

SEQ ID NO: 511 - protein, 33 aa, synthetic construct
  REGION 1..33: note = Synthetic
GSAPTVGRASEEANTTPASLTGPATSGSETPGT

SEQ ID NO: 512 - protein, 33 aa, synthetic construct
  REGION 1..33: note = Synthetic
GSAPTTGRAPEAANATPAPLTGPATSGSETPGT

SEQ ID NO: 513 - protein, 33 aa, synthetic construct
  REGION 1..33: note = Synthetic
GSAPTWGRATEPANATPAPLTSPATSGSETPGT

SEQ ID NO: 514 - protein, 33 aa, synthetic construct
  REGION 1..33: note = Synthetic
GSAPTVGRASESANATPAELTSPATSGSETPGT

SEQ ID NO: 515 - protein, 33 aa, synthetic construct
  REGION 1..33: note = Synthetic
GSAPTVGRAPEGANSTPAGLTGPATSGSETPGT

SEQ ID NO: 516 - protein, 33 aa, synthetic construct
  REGION 1..33: note = Synthetic
GSAPTWGRATEAPNLEPATLTTPATSGSETPGT

SEQ ID NO: 517 - protein, 33 aa, synthetic construct
  REGION 1..33: note = Synthetic
GSAPTTGRATEAPNLTPAPLTEPATSGSETPGT

SEQ ID NO: 518 - protein, 33 aa, synthetic construct
  REGION 1..33: note = Synthetic
GSAPTQGRATEAPNLSPAALTSPATSGSETPGT

SEQ ID NO: 519 - protein, 33 aa, synthetic construct
  REGION 1..33: note = Synthetic
GSAPTQGRAAEAPNLTPATLTAPATSGSETPGT

SEQ ID NO: 520 - protein, 33 aa, synthetic construct
  REGION 1..33: note = Synthetic
GSAPTSGRAPEATNLAPAPLTGPATSGSETPGT

SEQ ID NO: 521 - protein, 33 aa, synthetic construct
  REGION 1..33: note = Synthetic
GSAPTQGRAAEAANLTPAGLTEPATSGSETPGT

SEQ ID NO: 522 - protein, 33 aa, synthetic construct
  REGION 1..33: note = Synthetic
GSAPTTGRAGSAPNLPPTGLTTPATSGSETPGT

SEQ ID NO: 523 - protein, 33 aa, synthetic construct
  REGION 1..33: note = Synthetic
GSAPTTGRAGGAENLPPEGLTAPATSGSETPGT

SEQ ID NO: 524 - protein, 33 aa, synthetic construct
  REGION 1..33: note = Synthetic
GSAPTTSRAGTATNLTPEGLTAPATSGSETPGT

SEQ ID NO: 525 - protein, 33 aa, synthetic construct
  REGION 1..33: note = Synthetic
GSAPTTGRAGTATNLPPSGLTTPATSGSETPGT

SEQ ID NO: 526 - protein, 33 aa, synthetic construct
  REGION 1..33: note = Synthetic
GSAPTTARAGEAENLSPSGLTAPATSGSETPGT

SEQ ID NO: 527 - protein, 33 aa, synthetic construct
  REGION 1..33: note = Synthetic
GSAPTTGRAGGAGNLAPGGLTEPATSGSETPGT

SEQ ID NO: 528 - protein, 33 aa, synthetic construct
  REGION 1..33: note = Synthetic
GSAPTTGRAGTATNLPPEGLTGPATSGSETPGT

SEQ ID NO: 529 - protein, 33 aa, synthetic construct
  REGION 1..33: note = Synthetic
GSAPTTGRAGGAANLAPTGLTEPATSGSETPGT

SEQ ID NO: 530 - protein, 33 aa, synthetic construct
  REGION 1..33: note = Synthetic
GSAPTTGRAGTAENLAPSGLTTPATSGSETPGT

SEQ ID NO: 531 - protein, 33 aa, synthetic construct
  REGION 1..33: note = Synthetic
GSAPTTGRAGSATNLGPGGLTGPATSGSETPGT

SEQ ID NO: 532 - protein, 33 aa, synthetic construct
  REGION 1..33: note = Synthetic
GSAPTTARAGGAENLTPAGLTEPATSGSETPGT

SEQ ID NO: 533 - protein, 33 aa, synthetic construct
  REGION 1..33: note = Synthetic
GSAPTTARAGSAENLSPSGLTGPATSGSETPGT

SEQ ID NO: 534 - protein, 33 aa, synthetic construct
  REGION 1..33: note = Synthetic
GSAPTTARAGGAGNLAPEGLTTPATSGSETPGT

SEQ ID NO: 535 - protein, 33 aa, synthetic construct
  REGION 1..33: note = Synthetic
GSAPTTSRAGAAENLTPTGLTGPATSGSETPGT

SEQ ID NO: 536 - protein, 33 aa, synthetic construct
  REGION 1..33: note = Synthetic
GSAPTYGRTTTPGNEPPASLEAEATSGSETPGT

SEQ ID NO: 537 - protein, 33 aa, synthetic construct
  REGION 1..33: note = Synthetic
GSAPTYSRGESGPNEPPPGLTGPATSGSETPGT

SEQ ID NO: 538 - protein, 33 aa, synthetic construct
  REGION 1..33: note = Synthetic
GSAPAWGRTGASENETPAPLGGEATSGSETPGT

SEQ ID NO: 539 - protein, 33 aa, synthetic construct
  REGION 1..33: note = Synthetic
GSAPRWGRAETTPNTPPEGLETEATSGSETPGT

SEQ ID NO: 540 - protein, 33 aa, synthetic construct
  REGION 1..33: note = Synthetic
GSAPESGRAANHTGAEPPELGAGATSGSETPGT

SEQ ID NO: 541 - protein, 33 aa, synthetic construct
  REGION 1..33: note = Synthetic
GSAPTTGRAGEAANLTPAGLTESATSGSETPGT

SEQ ID NO: 542 - protein, 33 aa, synthetic construct
  REGION 1..33: note = Synthetic
GSAPTTGRAGEAANLTPAALTESATSGSETPGT

SEQ ID NO: 543 - protein, 33 aa, synthetic construct
  REGION 1..33: note = Synthetic
GSAPTTGRAGEAANLTPAPLTESATSGSETPGT

SEQ ID NO: 544 - protein, 33 aa, synthetic construct
  REGION 1..33: note = Synthetic
GSAPTTGRAGEAANLTPEPLTESATSGSETPGT

SEQ ID NO: 545 - protein, 33 aa, synthetic construct
  REGION 1..33: note = Synthetic
GSAPTTGRAGEAANLTPAGLTGAATSGSETPGT

SEQ ID NO: 546 - protein, 33 aa, synthetic construct
  REGION 1..33: note = Synthetic
GSAPTTGRAGEAANLTPEGLTGAATSGSETPGT

SEQ ID NO: 547 - protein, 33 aa, synthetic construct
  REGION 1..33: note = Synthetic
GSAPTTGRAGEAANLTPEPLTGAATSGSETPGT

SEQ ID NO: 548 - protein, 33 aa, synthetic construct
  REGION 1..33: note = Synthetic
GSAPTTGRAGEAANLTPAGLTEAATSGSETPGT

SEQ ID NO: 549 - protein, 33 aa, synthetic construct
  REGION 1..33: note = Synthetic
GSAPTTGRAGEAANLTPEGLTEAATSGSETPGT

SEQ ID NO: 550 - protein, 33 aa, synthetic construct
  REGION 1..33: note = Synthetic
GSAPTTGRAGEAANLTPAPLTEAATSGSETPGT

SEQ ID NO: 551 - protein, 33 aa, synthetic construct
  REGION 1..33: note = Synthetic
GSAPTTGRAGEAANLTPEPLTEAATSGSETPGT

SEQ ID NO: 552 - protein, 33 aa, synthetic construct
  REGION 1..33: note = Synthetic
GSAPTTGRAGEAANLTPEPLTGPATSGSETPGT

SEQ ID NO: 553 - protein, 33 aa, synthetic construct
  REGION 1..33: note = Synthetic
GSAPTTGRAGEAANLTPAGLTGGATSGSETPGT

SEQ ID NO: 554 - protein, 33 aa, synthetic construct
  REGION 1..33: note = Synthetic
GSAPTTGRAGEAANLTPEGLTGGATSGSETPGT

SEQ ID NO: 555 - protein, 33 aa, synthetic construct
  REGION 1..33: note = Synthetic
GSAPTTGRAGEAANLTPEALTGGATSGSETPGT

SEQ ID NO: 556 - protein, 33 aa, synthetic construct
  REGION 1..33: note = Synthetic
GSAPTTGRAGEAANLTPEPLTGGATSGSETPGT

SEQ ID NO: 557 - protein, 33 aa, synthetic construct
  REGION 1..33: note = Synthetic
GSAPTTGRAGEAANLTPAGLTEGATSGSETPGT

SEQ ID NO: 558 - protein, 33 aa, synthetic construct
  REGION 1..33: note = Synthetic
GSAPTTGRAGEAANLTPEGLTEGATSGSETPGT

SEQ ID NO: 559 - protein, 33 aa, synthetic construct
  REGION 1..33: note = Synthetic
GSAPTTGRAGEAANLTPAPLTEGATSGSETPGT

SEQ ID NO: 560 - protein, 33 aa, synthetic construct
  REGION 1..33: note = Synthetic
GSAPTTGRAGEAANLTPEPLTEGATSGSETPGT

SEQ ID NO: 561 - protein, 33 aa, synthetic construct; identical to SEQ ID NO: 484
  REGION 1..33: note = Synthetic

SEQ ID NO: 562 - protein, 30 aa, synthetic construct
  REGION 1..30: note = Synthetic
GSAPEAGRSAEATSAGATGPATSGSETPGT

SEQ ID NO: 563 - protein, 30 aa, synthetic construct
  REGION 1..30: note = Synthetic
GSAPSASGTYSRGESGPGSPATSGSETPGT

SEQ ID NO: 564 - protein, 30 aa, synthetic construct
  REGION 1..30: note = Synthetic
GSAPSASGEAGRTDTHPGSPATSGSETPGT

SEQ ID NO: 565 - protein, 30 aa, synthetic construct
  REGION 1..30: note = Synthetic
GSAPSASGEPGRAAEHPGSPATSGSETPGT

SEQ ID NO: 566 - protein, 30 aa, synthetic construct
  REGION 1..30: note = Synthetic
GSAPSPAGESSRGTTIAGSPATSGSETPGT

SEQ ID NO: 567 - protein, 33 aa, synthetic construct; identical to SEQ ID NO: 486
  REGION 1..33: note = Synthetic

SEQ ID NO: 568 - protein, 30 aa, synthetic construct
  REGION 1..30: note = Synthetic
GSAPEAGESAGATPAGLTGPATSGSETPGT

SEQ ID NO: 569 - protein, 30 aa, synthetic construct
  REGION 1..30: note = Synthetic
GSAPSASGAPLELEAGPGSPATSGSETPGT

SEQ ID NO: 570 - protein, 30 aa, synthetic construct
  REGION 1..30: note = Synthetic
GSAPSASGEPPELGAGPGSPATSGSETPGT

SEQ ID NO: 571 - protein, 30 aa, synthetic construct
  REGION 1..30: note = Synthetic
GSAPSASGEPSGLTEGPGSPATSGSETPGT

SEQ ID NO: 572 - protein, 30 aa, synthetic construct
  REGION 1..30: note = Synthetic
GSAPSASGTPAPLTEPPGSPATSGSETPGT

SEQ ID NO: 573 - protein, 30 aa, synthetic construct
  REGION 1..30: note = Synthetic
GSAPSASGTPAELTEPPGSPATSGSETPGT

SEQ ID NO: 574 - protein, 30 aa, synthetic construct
  REGION 1..30: note = Synthetic
GSAPSASGPPPGLTGPPGSPATSGSETPGT

SEQ ID NO: 575 - protein, 30 aa, synthetic construct
  REGION 1..30: note = Synthetic
GSAPSASGTPAPLGGEPGSPATSGSETPGT

SEQ ID NO: 576 - protein, 30 aa, synthetic construct
  REGION 1..30: note = Synthetic
GSAPSPAGAPEGLTGPAGSPATSGSETPGT

SEQ ID NO: 577 - protein, 30 aa, synthetic construct
  REGION 1..30: note = Synthetic
GSAPSPAGPPEGLETEAGSPATSGSETPGT

SEQ ID NO: 578 - protein, 30 aa, synthetic construct
  REGION 1..30: note = Synthetic
GSAPSPTSGQGGLTGPGSEPATSGSETPGT

SEQ ID NO: 579 - protein, 30 aa, synthetic construct
  REGION 1..30: note = Synthetic
GSAPSESAPPEGLETESTEPATSGSETPGT

SEQ ID NO: 580 - protein, 30 aa, synthetic construct
  REGION 1..30: note = Synthetic
GSAPSEGSEPLELGAASETPATSGSETPGT

SEQ ID NO: 581 - protein, 30 aa, synthetic construct
  REGION 1..30: note = Synthetic
GSAPSEGSGPAGLEAPSETPATSGSETPGT

SEQ ID NO: 582 - protein, 30 aa, synthetic construct
  REGION 1..30: note = Synthetic
GSAPSEPTPPASLEAEPGSPATSGSETPGT

SEQ ID NO: 583 - protein, 29 aa, synthetic construct
  REGION 1..29: note = Synthetic
GTAEAASASGGSAGGYAELRMGGAIPGSP

SEQ ID NO: 584 - protein, 29 aa, synthetic construct
  REGION 1..29: note = Synthetic
GTAEAASASGGTGGGYAPLRMGGGAPGSP

SEQ ID NO: 585 - protein, 29 aa, synthetic construct
  REGION 1..29: note = Synthetic
GTAEAASASGGAEGGYAALRMGGEIPGSP

SEQ ID NO: 586 - protein, 29 aa, synthetic construct
  REGION 1..29: note = Synthetic
GTAEAASASGGGPGGYALLRMGGPAPGSP

SEQ ID NO: 587 - protein, 29 aa, synthetic construct
  REGION 1..29: note = Synthetic
GTAEAASASGGEAGGYAFLRMGGSIPGSP

SEQ ID NO: 588 - protein, 29 aa, synthetic construct
  REGION 1..29: note = Synthetic
GTAEAASASGGPGGGYASLRMGGTAPGSP

SEQ ID NO: 589 - protein, 29 aa, synthetic construct
  REGION 1..29: note = Synthetic
GTAEAASASGGSEGGYATLRMGGAIPGSP

SEQ ID NO: 590 - protein, 29 aa, synthetic construct
  REGION 1..29: note = Synthetic
GTAEAASASGGTPGGYANLRMGGGAPGSP

SEQ ID NO: 591 - protein, 29 aa, synthetic construct
  REGION 1..29: note = Synthetic
GTAEAASASGGASGGYAHLRMGGEIPGSP

SEQ ID NO: 592 - protein, 29 aa, synthetic construct
  REGION 1..29: note = Synthetic
GTAEAASASGGGTGGYGELRMGGPAPGSP

SEQ ID NO: 593 - protein, 29 aa, synthetic construct
  REGION 1..29: note = Synthetic
GTAEAASASGGEAGGYPELRMGGSIPGSP

SEQ ID NO: 594 - protein, 29 aa, synthetic construct
  REGION 1..29: note = Synthetic
GTAEAASASGGPGGGYVELRMGGTAPGSP

SEQ ID NO: 595 - protein, 29 aa, synthetic construct
  REGION 1..29: note = Synthetic
GTAEAASASGGSEGGYLELRMGGAIPGSP

SEQ ID NO: 596 - protein, 29 aa, synthetic construct
  REGION 1..29: note = Synthetic
GTAEAASASGGTPGGYSELRMGGGAPGSP

SEQ ID NO: 597 - protein, 29 aa, synthetic construct
  REGION 1..29: note = Synthetic
GTAEAASASGGASGGYTELRMGGEIPGSP

SEQ ID NO: 598 - protein, 29 aa, synthetic construct
  REGION 1..29: note = Synthetic
GTAEAASASGGGTGGYQELRMGGPAPGSP

SEQ ID NO: 599 - protein, 29 aa, synthetic construct
  REGION 1..29: note = Synthetic
GTAEAASASGGEAGGYEELRMGGSIPGSP

SEQ ID NO: 600 - protein, 29 aa, synthetic construct
  REGION 1..29: note = Synthetic
GTAEAASASGGPGIGPAELRMGGTAPGSP

SEQ ID NO: 601 - protein, 29 aa, synthetic construct
  REGION 1..29: note = Synthetic
GTAEAASASGGSEIGAAELRMGGAIPGSP

SEQ ID NO: 602 - protein, 29 aa, synthetic construct
  REGION 1..29: note = Synthetic
GTAEAASASGGTPIGSAELRMGGGAPGSP

SEQ ID NO: 603 - protein, 29 aa, synthetic construct
  REGION 1..29: note = Synthetic
GTAEAASASGGASIGTAELRMGGEIPGSP

SEQ ID NO: 604 - protein, 29 aa, synthetic construct
  REGION 1..29: note = Synthetic
GTAEAASASGGGTIGNAELRMGGPAPGSP

SEQ ID NO: 605 - protein, 29 aa, synthetic construct
  REGION 1..29: note = Synthetic
GTAEAASASGGEAIGQAELRMGGSIPGSP

SEQ ID NO: 606 - protein, 29 aa, synthetic construct
  REGION 1..29: note = Synthetic
GTAEAASASGGPGGPYAELRMGGTAPGSP

SEQ ID NO: 607 - protein, 29 aa, synthetic construct
  REGION 1..29: note = Synthetic
GTAEAASASGGSEGAYAELRMGGAIPGSP

SEQ ID NO: 608 - protein, 29 aa, synthetic construct
  REGION 1..29: note = Synthetic
GTAEAASASGGTPGVYAELRMGGGAPGSP

SEQ ID NO: 609 - protein, 29 aa, synthetic construct
  REGION 1..29: note = Synthetic
GTAEAASASGGASGLYAELRMGGEIPGSP

SEQ ID NO: 610 - protein, 29 aa, synthetic construct
  REGION 1..29: note = Synthetic
GTAEAASASGGGTGIYAELRMGGPAPGSP

SEQ ID NO: 611 - protein, 29 aa, synthetic construct
  REGION 1..29: note = Synthetic
GTAEAASASGGEAGFYAELRMGGSIPGSP

SEQ ID NO: 612 - protein, 29 aa, synthetic construct
  REGION 1..29: note = Synthetic
GTAEAASASGGPGGYYAELRMGGTAPGSP

SEQ ID NO: 613 - protein, 29 aa, synthetic construct
  REGION 1..29: note = Synthetic
GTAEAASASGGSEGSYAELRMGGAIPGSP

SEQ ID NO: 614 - protein, 29 aa, synthetic construct
  REGION 1..29: note = Synthetic
GTAEAASASGGTPGNYAELRMGGGAPGSP

SEQ ID NO: 615 - protein, 29 aa, synthetic construct
  REGION 1..29: note = Synthetic
GTAEAASASGGASGEYAELRMGGEIPGSP

SEQ ID NO: 616 - protein, 29 aa, synthetic construct
  REGION 1..29: note = Synthetic
GTAEAASASGGGTGHYAELRMGGPAPGSP

SEQ ID NO: 617 - protein, 29 aa, synthetic construct
  REGION 1..29: note = Synthetic
GTAEAASASGGEAGGYAEARMGGSIPGSP

SEQ ID NO: 618 - protein, 29 aa, synthetic construct
  REGION 1..29: note = Synthetic
GTAEAASASGGPGGGYAEVRMGGTAPGSP

SEQ ID NO: 619 - protein, 29 aa, synthetic construct
  REGION 1..29: note = Synthetic
GTAEAASASGGSEGGYAEIRMGGAIPGSP

SEQ ID NO: 620 - protein, 29 aa, synthetic construct
  REGION 1..29: note = Synthetic
GTAEAASASGGTPGGYAEFRMGGGAPGSP

SEQ ID NO: 621 - protein, 29 aa, synthetic construct
  REGION 1..29: note = Synthetic
GTAEAASASGGASGGYAEYRMGGEIPGSP

SEQ ID NO: 622 - protein, 29 aa, synthetic construct
  REGION 1..29: note = Synthetic
GTAEAASASGGGTGGYAESRMGGPAPGSP

SEQ ID NO: 623 - protein, 29 aa, synthetic construct
  REGION 1..29: note = Synthetic
GTAEAASASGGEAGGYAETRMGGSIPGSP

SEQ ID NO: 624 - protein, 29 aa, synthetic construct
  REGION 1..29: note = Synthetic
GTAEAASASGGPGGGYAELAMGGTRPGSP

SEQ ID NO: 625 - protein, 29 aa, synthetic construct
  REGION 1..29: note = Synthetic
GTAEAASASGGSEGGYAELVMGGARPGSP

SEQ ID NO: 626 - protein, 29 aa, synthetic construct
  REGION 1..29: note = Synthetic
GTAEAASASGGTPGGYAELLMGGGRPGSP

SEQ ID NO: 627 - protein, 29 aa, synthetic construct
  REGION 1..29: note = Synthetic
GTAEAASASGGASGGYAELIMGGERPGSP

SEQ ID NO: 628 - protein, 29 aa, synthetic construct
  REGION 1..29: note = Synthetic
GTAEAASASGGGTGGYAELWMGGPRPGSP

SEQ ID NO: 629 - protein, 29 aa, synthetic construct
  REGION 1..29: note = Synthetic
GTAEAASASGGEAGGYAELSMGGSRPGSP

SEQ ID NO: 630 - protein, 29 aa, synthetic construct
  REGION 1..29: note = Synthetic
GTAEAASASGGPGGGYAELTMGGTRPGSP

SEQ ID NO: 631 - protein, 29 aa, synthetic construct
  REGION 1..29: note = Synthetic
GTAEAASASGGSEGGYAELQMGGARPGSP

SEQ ID NO: 632 - protein, 29 aa, synthetic construct
  REGION 1..29: note = Synthetic
GTAEAASASGGTPGGYAELNMGGGRPGSP

SEQ ID NO: 633 - protein, 29 aa, synthetic construct
  REGION 1..29: note = Synthetic
GTAEAASASGGASGGYAELEMGGERPGSP

SEQ ID NO: 634 - protein, 29 aa, synthetic construct
  REGION 1..29: note = Synthetic
GTAEAASASGGGTGGYAELRPGGPIPGSP

SEQ ID NO: 635 - protein, 29 aa, synthetic construct
  REGION 1..29: note = Synthetic
GTAEAASASGGEAGGYAELRAGGSAPGSP

SEQ ID NO: 636 - protein, 29 aa, synthetic construct
  REGION 1..29: note = Synthetic
GTAEAASASGGPGGGYAELRLGGTIPGSP

SEQ ID NO: 637 - protein, 29 aa, synthetic construct
  REGION 1..29: note = Synthetic
GTAEAASASGGSEGGYAELRIGGAAPGSP

SEQ ID NO: 638 - protein, 29 aa, synthetic construct
  REGION 1..29: note = Synthetic
GTAEAASASGGTPGGYAELRSGGGIPGSP

SEQ ID NO: 639 - protein, 29 aa, synthetic construct
  REGION 1..29: note = Synthetic
GTAEAASASGGASGGYAELRNGGEAPGSP

SEQ ID NO: 640 - protein, 29 aa, synthetic construct
  REGION 1..29: note = Synthetic
GTAEAASASGGGTGGYAELRQGGPIPGSP

SEQ ID NO: 641 - protein, 29 aa, synthetic construct
  REGION 1..29: note = Synthetic
GTAEAASASGGEAGGYAELRDGGSAPGSP

SEQ ID NO: 642 - protein, 29 aa, synthetic construct
  REGION 1..29: note = Synthetic
GTAEAASASGGPGGGYAELREGGTIPGSP

SEQ ID NO: 643 - protein, 29 aa, synthetic construct
  REGION 1..29: note = Synthetic
GTAEAASASGGSEGGYAELRHGGAAPGSP

SEQ ID NO: 644 - protein, 29 aa, synthetic construct
  REGION 1..29: note = Synthetic
GTAEAASASGGTPGGYAELRMPGGIPGSP

SEQ ID NO: 645 - protein, 29 aa, synthetic construct
  REGION 1..29: note = Synthetic
GTAEAASASGGASGGYAELRMAGEAPGSP

SEQ ID NO: 646 - protein, 29 aa, synthetic construct
  REGION 1..29: note = Synthetic
GTAEAASASGGGTGGYAELRMVGPIPGSP

SEQ ID NO: 647 - protein, 29 aa, synthetic construct
  REGION 1..29: note = Synthetic
GTAEAASASGGEAGGYAELRMLGSAPGSP

SEQ ID NO: 648 - protein, 29 aa, synthetic construct
  REGION 1..29: note = Synthetic
GTAEAASASGGPGGGYAELRMIGTIPGSP

SEQ ID NO: 649 - protein, 29 aa, synthetic construct
  REGION 1..29: note = Synthetic
GTAEAASASGGSEGGYAELRMYGAIPGSP

SEQ ID NO: 650 - protein, 29 aa, synthetic construct
  REGION 1..29: note = Synthetic
GTAEAASASGGTPGGYAELRMSGGAPGSP

SEQ ID NO: 651 - protein, 29 aa, synthetic construct
  REGION 1..29: note = Synthetic
GTAEAASASGGASGGYAELRMNGEIPGSP

SEQ ID NO: 652 - protein, 29 aa, synthetic construct
  REGION 1..29: note = Synthetic
GTAEAASASGGGTGGYAELRMQGPAPGSP

SEQ ID NO: 653 - protein, 29 aa, synthetic construct
  REGION 1..29: note = Synthetic
GTAEAASASGGANHTPAGLTGPGARPGSP

SEQ ID NO: 654 - protein, 29 aa, synthetic construct
  REGION 1..29: note = Synthetic
GTAEAASASGGANTAPEGLTGPSTRPGSP

SEQ ID NO: 655 - protein, 29 aa, synthetic construct
  REGION 1..29: note = Synthetic
GTAEAASASGGTGAPPGGLTGPGTRPGSP

SEQ ID NO: 656 - protein, 29 aa, synthetic construct
  REGION 1..29: note = Synthetic
GTAEAASASGGANHEPSGLTEGSPRPGSP

SEQ ID NO: 657 - protein, 29 aa, synthetic construct
  REGION 1..29: note = Synthetic
GTAEAASASGGANTEPPELGAGTERPGSP

SEQ ID NO: 658 - protein, 29 aa, synthetic construct
  REGION 1..29: note = Synthetic
GTAEAASASGGASGPPPGLTGPPGRPGSP

SEQ ID NO: 659 - protein, 29 aa, synthetic construct
  REGION 1..29: note = Synthetic
GTAEAASASGGASGTPAPLGGEPGRPGSP

SEQ ID NO: 660 - protein, 29 aa, synthetic construct
  REGION 1..29: note = Synthetic
GTAEAASASGGPAGPPEGLETEAGRPGSP

SEQ ID NO: 661 - protein, 29 aa, synthetic construct
  REGION 1..29: note = Synthetic
GTAEAASASGGPTSGQGGLTGPESRPGSP

SEQ ID NO: 662 - protein, 29 aa, synthetic construct
  REGION 1..29: note = Synthetic
GTAEAASASGGSAGGAANLVRGGAIPGSP

SEQ ID NO: 663 - protein, 29 aa, synthetic construct
  REGION 1..29: note = Synthetic
GTAEAASASGGTGGGAAPLVRGGGAPGSP

SEQ ID NO: 664 - protein, 29 aa, synthetic construct
  REGION 1..29: note = Synthetic
GTAEAASASGGAEGGAAALVRGGEIPGSP

SEQ ID NO: 665 - protein, 29 aa, synthetic construct
  REGION 1..29: note = Synthetic
GTAEAASASGGGPGGAALLVRGGPAPGSP

SEQ ID NO: 666 - protein, 29 aa, synthetic construct
  REGION 1..29: note = Synthetic
GTAEAASASGGEAGGAAFLVRGGSIPGSP

SEQ ID NO: 667 - protein, 29 aa, synthetic construct
  REGION 1..29: note = Synthetic
GTAEAASASGGPGGGAASLVRGGTAPGSP

SEQ ID NO: 668 - protein, 29 aa, synthetic construct
  REGION 1..29: note = Synthetic
GTAEAASASGGSEGGAATLVRGGAIPGSP

SEQ ID NO: 669 - protein, 29 aa, synthetic construct
  REGION 1..29: note = Synthetic
GTAEAASASGGTPGGAAGLVRGGGAPGSP

SEQ ID NO: 670 - protein, 29 aa, synthetic construct
  REGION 1..29: note = Synthetic
GTAEAASASGGASGGAADLVRGGEIPGSP

SEQ ID NO: 671 - protein, 29 aa, synthetic construct
  REGION 1..29: note = Synthetic
GTAEAASASGGGTGGAGNLVRGGPAPGSP

SEQ ID NO: 672 - protein, 29 aa, synthetic construct
  REGION 1..29: note = Synthetic
GTAEAASASGGEAGGAPNLVRGGSIPGSP

SEQ ID NO: 673 - protein, 29 aa, synthetic construct
  REGION 1..29: note = Synthetic
GTAEAASASGGPGGGAVNLVRGGTAPGSP

SEQ ID NO: 674 - protein, 29 aa, synthetic construct
  REGION 1..29: note = Synthetic
GTAEAASASGGSEGGALNLVRGGAIPGSP

SEQ ID NO: 675 - protein, 29 aa, synthetic construct
  REGION 1..29: note = Synthetic
GTAEAASASGGTPGGASNLVRGGGAPGSP

SEQ ID NO: 676 - protein, 29 aa, synthetic construct
  REGION 1..29: note = Synthetic
GTAEAASASGGASGGATNLVRGGEIPGSP

SEQ ID NO: 677 - protein, 29 aa, synthetic construct
  REGION 1..29: note = Synthetic
GTAEAASASGGGTGGAQNLVRGGPAPGSP

SEQ ID NO: 678 - protein, 29 aa, synthetic construct
  REGION 1..29: note = Synthetic
GTAEAASASGGEAGGAENLVRGGSIPGSP

SEQ ID NO: 679 - protein, 30 aa, synthetic construct
  REGION 1..30: note = Synthetic
GTAEAASASGEAGRSANHEPLGLVATPGSP

SEQ ID NO: 680 - protein, 30 aa, synthetic construct
  REGION 1..30: note = Synthetic
GTAEAASASGASGRSTNAGPSGLAGPPGSP

SEQ ID NO: 681 - protein, 30 aa, synthetic construct
  REGION 1..30: note = Synthetic
GTAEAASASGASGRSTNAGPQGLAGQPGSP

SEQ ID NO: 682 - protein, 30 aa, synthetic construct
  REGION 1..30: note = Synthetic
GTAEAASASGASGRSTNAGPPGLTGPPGSP

SEQ ID NO: 683 - protein, 30 aa, synthetic construct
  REGION 1..30: note = Synthetic
GTAEAASASGASSRGTNAGPAGLTGPPGSP

SEQ ID NO: 684 - protein, 30 aa, synthetic construct
  REGION 1..30: note = Synthetic
GTAEAASASGASSRTTNTGPSTLTGPPGSP

SEQ ID NO: 685 - protein, 30 aa, synthetic construct
  REGION 1..30: note = Synthetic
GTAEAASASGAAGRSDNGTPLELVAPPGSP

SEQ ID NO: 686 - protein, 30 aa, synthetic construct; identical to SEQ ID NO: 679
  REGION 1..30: note = Synthetic

SEQ ID NO: 687 - protein, 30 aa, synthetic construct
  REGION 1..30: note = Synthetic
GTAEAASASGASGRGTNAGPAGLTGPPGSP

SEQ ID NO: 688 - protein, 30 aa, synthetic construct
  REGION 1..30: note = Synthetic
GTAEAASASGLFGRNDNHEPLELGGGPGSP

SEQ ID NO: 689 - protein, 30 aa, synthetic construct
  REGION 1..30: note = Synthetic
GTAEAASASGTAGRSDNLEPLGLVFGPGSP

SEQ ID NO: 690 - protein, 30 aa, synthetic construct
  REGION 1..30: note = Synthetic
GTAEAASASGLDGRSDNFHPPELVAGPGSP

SEQ ID NO: 691 - protein, 30 aa, synthetic construct
  REGION 1..30: note = Synthetic
GTAEAASASGLEGRSDNEEPENLVAGPGSP

SEQ ID NO: 692 - protein, 30 aa, synthetic construct
  REGION 1..30: note = Synthetic
GTAEAASASGLKGRSDNNAPLALVAGPGSP

SEQ ID NO: 693 - protein, 30 aa, synthetic construct
  REGION 1..30: note = Synthetic
GTAEAASASGVYSRGTNAGPHGLTGRPGSP

SEQ ID NO: 694 - protein, 30 aa, synthetic construct
  REGION 1..30: note = Synthetic
GTAEAASASGANSRGTNKGFAGLIGPPGSP

SEQ ID NO: 695 - protein, 30 aa, synthetic construct
  REGION 1..30: note = Synthetic
GTAEAASASGASSRLTNEAPAGLTIPPGSP

SEQ ID NO: 696 - protein, 30 aa, synthetic construct
  REGION 1..30: note = Synthetic
GTAEAASASGDQSRGTNAGPEGLTDPPGSP

SEQ ID NO: 697 - protein, 30 aa, synthetic construct
  REGION 1..30: note = Synthetic
GTAEAASASGESSRGTNIGQGGLTGPPGSP

SEQ ID NO: 698 - protein, 30 aa, synthetic construct
  REGION 1..30: note = Synthetic
GTAEAASASGSSSRGTNQDPAGLTIPPGSP

SEQ ID NO: 699 - protein, 30 aa, synthetic construct
  REGION 1..30: note = Synthetic
GTAEAASASGASSRGQNHSPMGLTGPPGSP

SEQ ID NO: 700 - protein, 30 aa, synthetic construct
  REGION 1..30: note = Synthetic
GTAEAASASGAYSRGPNAGPAGLEGRPGSP

SEQ ID NO: 701 - protein, 30 aa, synthetic construct
  REGION 1..30: note = Synthetic
GTAEAASASGASERGNNAGPANLTGFPGSP

SEQ ID NO: 702 - protein, 30 aa, synthetic construct
  REGION 1..30: note = Synthetic
GTAEAASASGASHRGTNPKPAILTGPPGSP

SEQ ID NO: 703 - protein, 30 aa, synthetic construct
  REGION 1..30: note = Synthetic
GTAEAASASGMSSRRTNANPAQLTGPPGSP

SEQ ID NO: 704 - protein, 30 aa, synthetic construct
  REGION 1..30: note = Synthetic
GTAEAASASGGAGRTDNHEPLELGAAPGSP

SEQ ID NO: 705 - protein, 30 aa, synthetic construct
  REGION 1..30: note = Synthetic
GTAEAASASGLAGRSENTAPLELTAGPGSP

SEQ ID NO: 706 - protein, 30 aa, synthetic construct
  REGION 1..30: note = Synthetic
GTAEAASASGLEGRPDNHEPLALVASPGSP

SEQ ID NO: 707 - protein, 30 aa, synthetic construct
  REGION 1..30: note = Synthetic
GTAEAASASGLSGRSDNEEPLALPAGPGSP

SEQ ID NO: 708 - protein, 30 aa, synthetic construct
  REGION 1..30: note = Synthetic
GTAEAASASGEAGRTDNHEPLELSAPPGSP

SEQ ID NO: 709 - protein, 30 aa, synthetic construct
  REGION 1..30: note = Synthetic
GTAEAASASGEGGRSDNHGPLELVSGPGSP

SEQ ID NO: 710 - protein, 30 aa, synthetic construct
  REGION 1..30: note = Synthetic
GTAEAASASGLSGRSDNEAPLELEAGPGSP

SEQ ID NO: 711 - protein, 30 aa, synthetic construct
  REGION 1..30: note = Synthetic
GTAEAASASGLGGRADNHEPPELGAGPGSP

SEQ ID NO: 712 - protein, 30 aa, synthetic construct
  REGION 1..30: note = Synthetic
GTAEAASASGPPSRGTNAEPAGLTGEPGSP

SEQ ID NO: 713 - protein, 30 aa, synthetic construct
  REGION 1..30: note = Synthetic
GTAEAASASGASTRGENAGPAGLEAPPGSP

SEQ ID NO: 714 - protein, 30 aa, synthetic construct
  REGION 1..30: note = Synthetic
GTAEAASASGESSRGTNGAPEGLTGPPGSP

SEQ ID NO: 715 - protein, 30 aa, synthetic construct
  REGION 1..30: note = Synthetic
GTAEAASASGASSRATNESPAGLTGEPGSP

SEQ ID NO: 716 - protein, 30 aa, synthetic construct
  REGION 1..30: note = Synthetic
GTAEAASASGASSRGENPPPGGLTGPPGSP

SEQ ID NO: 717 - protein, 30 aa, synthetic construct
  REGION 1..30: note = Synthetic
GTAEAASASGAASRGTNTGPAELTGSPGSP

SEQ ID NO: 718 - protein, 30 aa, synthetic construct
  REGION 1..30: note = Synthetic
GTAEAASASGAGSRTTNAGPGGLEGPPGSP

SEQ ID NO: 719 - protein, 30 aa, synthetic construct
  REGION 1..30: note = Synthetic
GTAEAASASGAPSRGENAGPATLTGAPGSP

SEQ ID NO: 720 - protein, 30 aa, synthetic construct
  REGION 1..30: note = Synthetic
GTAEAASASGESGRAANTGPPTLTAPPGSP

SEQ ID NO: 721 - protein, 30 aa, synthetic construct
  REGION 1..30: note = Synthetic
GTAEAASASGNPGRAANEGPPGLPGSPGSP

SEQ ID NO: 722 - protein, 30 aa, synthetic construct
  REGION 1..30: note = Synthetic
GTAEAASASGESSRAANLTPPELTGPPGSP

SEQ ID NO: 723 - protein, 30 aa, synthetic construct
  REGION 1..30: note = Synthetic
GTAEAASASGASGRAANETPPGLTGAPGSP

SEQ ID NO: 724 - protein, 30 aa, synthetic construct
  REGION 1..30: note = Synthetic
GTAEAASASGNSGRGENLGAPGLTGTPGSP

SEQ ID NO: 725 - protein, 30 aa, synthetic construct
  REGION 1..30: note = Synthetic
GTAEAASASGTTGRAANLTPAGLTGPPGSP

SEQ ID NO: 726 - protein, 30 aa, synthetic construct
  REGION 1..30: note = Synthetic
GTAEAASASGEAGRSANHTPAGLTGPPGSP

SEQ ID NO: 727 - protein, 30 aa, synthetic construct
  REGION 1..30: note = Synthetic
GTAEAASASGESGRAANTTPAGLTGPPGSP

SEQ ID NO: 728 - protein, 33 aa, synthetic construct
  REGION 1..33: note = Synthetic
GTAEAASASGTTGRATEAANLTPAGLTGPPGSP

SEQ ID NO: 729 - protein, 33 aa, synthetic construct
  REGION 1..33: note = Synthetic
GTAEAASASGTTGRAEEAANLTPAGLTGPPGSP

SEQ ID NO: 730 - protein, 33 aa, synthetic construct
  REGION 1..33: note = Synthetic
GTAEAASASGTTGRAGEAANLTPAGLTGPPGSP

SEQ ID NO: 731 - protein, 33 aa, synthetic construct
  REGION 1..33: note = Synthetic
GTAEAASASGTTGRATEAANATPAGLTGPPGSP

SEQ ID NO: 732 - protein, 33 aa, synthetic construct
  REGION 1..33: note = Synthetic
GTAEAASASGTTGRAGEAEGATSAGATGPPGSP

SEQ ID NO: 733 - protein, 33 aa, synthetic construct
  REGION 1..33: note = Synthetic
GTAEAASASGTTGEAGEAANATSAGATGPPGSP

SEQ ID NO: 734 - protein, 33 aa, synthetic construct
  REGION 1..33: note = Synthetic
GTAEAASASGTTGEAGEAAGLTPAGLTGPPGSP

SEQ ID NO: 735 - protein, 33 aa, synthetic construct
  REGION 1..33: note = Synthetic
GTAEAASASGTTGAAGEAANATPAGLTGPPGSP

SEQ ID NO: 736 - protein, 33 aa, synthetic construct
  REGION 1..33: note = Synthetic
GTAEAASASGTTGRAGEAAGLTPAGLTGPPGSP

SEQ ID NO: 737 - protein, 33 aa, synthetic construct
  REGION 1..33: note = Synthetic
GTAEAASASGTTGRAGEAANATSAGATGPPGSP

SEQ ID NO: 738 - protein, 33 aa, synthetic construct
  REGION 1..33: note = Synthetic
GTAEAASASGTTGEAGEAAGATSAGATGPPGSP

SEQ ID NO: 739 - protein, 30 aa, synthetic construct
  REGION 1..30: note = Synthetic
GTAEAASASGESGRAANTEPPELGAGPGSP

SEQ ID NO: 740 - protein, 30 aa, synthetic construct
  REGION 1..30: note = Synthetic
GTAEAASASGESGRAANTAPEGLTGPPGSP

SEQ ID NO: 741 - protein, 30 aa, synthetic construct
  REGION 1..30: note = Synthetic
GTAEAASASGEPGRAANHEPSGLTEGPGSP

SEQ ID NO: 742 - protein, 33 aa, synthetic construct
  REGION 1..33: note = Synthetic
GTAEAASASGESGRAANHTGAPPGGLTGPPGSP

SEQ ID NO: 743 - protein, 33 aa, synthetic construct
  REGION 1..33: note = Synthetic
GTAEAASASGTTGRTGEGANATPGGLTGPPGSP

SEQ ID NO: 744 - protein, 33 aa, synthetic construct
  REGION 1..33: note = Synthetic
GTAEAASASGRTGRSGEAANETPEGLEGPPGSP

SEQ ID NO: 745 - protein, 33 aa, synthetic construct
  REGION 1..33: note = Synthetic
GTAEAASASGRTGRTGESANETPAGLGGPPGSP

SEQ ID NO: 746 - protein, 33 aa, synthetic construct
  REGION 1..33: note = Synthetic
GTAEAASASGSTGRTGEPANETPAGLSGPPGSP

SEQ ID NO: 747 - protein, 33 aa, synthetic construct
  REGION 1..33: note = Synthetic
GTAEAASASGTTGRAGEPANATPTGLSGPPGSP

SEQ ID NO: 748 - protein, 33 aa, synthetic construct
  REGION 1..33: note = Synthetic
GTAEAASASGRTGRPGEGANATPTGLPGPPGSP

SEQ ID NO: 749 - protein, 33 aa, synthetic construct
  REGION 1..33: note = Synthetic
GTAEAASASGRTGRGGEAANATPSGLGGPPGSP

SEQ ID NO: 750 - protein, 33 aa, synthetic construct
  REGION 1..33: note = Synthetic
GTAEAASASGSTGRSGESANATPGGLGGPPGSP

SEQ ID NO: 751 - protein, 33 aa, synthetic construct
  REGION 1..33: note = Synthetic
GTAEAASASGRTGRTGEEANATPAGLPGPPGSP

SEQ ID NO: 752 - protein, 33 aa, synthetic construct
  REGION 1..33: note = Synthetic
GTAEAASASGATGRPGEPANTTPEGLEGPPGSP

SEQ ID NO: 753 - protein, 33 aa, synthetic construct
  REGION 1..33: note = Synthetic
GTAEAASASGSTGRSGEPANATPGGLTGPPGSP

SEQ ID NO: 754 - protein, 33 aa, synthetic construct
  REGION 1..33: note = Synthetic
GTAEAASASGPTGRGGEGANTTPTGLPGPPGSP

SEQ ID NO: 755 - protein, 33 aa, synthetic construct
  REGION 1..33: note = Synthetic
GTAEAASASGPTGRSGEGANATPSGLTGPPGSP

SEQ ID NO: 756 - protein, 33 aa, synthetic construct
  REGION 1..33: note = Synthetic
GTAEAASASGTTGRASEGANSTPAPLTEPPGSP

SEQ ID NO: 757 - protein, 33 aa, synthetic construct
  REGION 1..33: note = Synthetic
GTAEAASASGTYGRAAEAANTTPAGLTAPPGSP

SEQ ID NO: 758 - protein, 33 aa, synthetic construct
  REGION 1..33: note = Synthetic
GTAEAASASGTTGRATEGANATPAELTEPPGSP

SEQ ID NO: 759 - protein, 33 aa, synthetic construct
  REGION 1..33: note = Synthetic
GTAEAASASGTVGRASEEANTTPASLTGPPGSP

SEQ ID NO: 760 - protein, 33 aa, synthetic construct
  REGION 1..33: note = Synthetic
GTAEAASASGTTGRAPEAANATPAPLTGPPGSP

SEQ ID NO: 761 - protein, 33 aa, synthetic construct
  REGION 1..33: note = Synthetic
GTAEAASASGTWGRATEPANATPAPLTSPPGSP

SEQ ID NO: 762 - protein, 33 aa, synthetic construct
  REGION 1..33: note = Synthetic
GTAEAASASGTVGRASESANATPAELTSPPGSP

SEQ ID NO: 763 - protein, 33 aa, synthetic construct
  REGION 1..33: note = Synthetic
GTAEAASASGTVGRAPEGANSTPAGLTGPPGSP

SEQ ID NO: 764 - protein, 33 aa, synthetic construct
  REGION 1..33: note = Synthetic
GTAEAASASGTWGRATEAPNLEPATLTTPPGSP

SEQ ID NO: 765 - protein, 33 aa, synthetic construct
  REGION 1..33: note = Synthetic
GTAEAASASGTTGRATEAPNLTPAPLTEPPGSP

SEQ ID NO: 766 - protein, 33 aa, synthetic construct
  REGION 1..33: note = Synthetic
GTAEAASASGTQGRATEAPNLSPAALTSPPGSP

SEQ ID NO: 767 - protein, 33 aa, synthetic construct
  REGION 1..33: note = Synthetic
GTAEAASASGTQGRAAEAPNLTPATLTAPPGSP

SEQ ID NO: 768 - protein, 33 aa, synthetic construct
  REGION 1..33: note = Synthetic
GTAEAASASGTSGRAPEATNLAPAPLTGPPGSP

SEQ ID NO: 769 - protein, 33 aa, synthetic construct
  REGION 1..33: note = Synthetic
GTAEAASASGTQGRAAEAANLTPAGLTEPPGSP

SEQ ID NO: 770 - protein, 33 aa, synthetic construct
  REGION 1..33: note = Synthetic
GTAEAASASGTTGRAGSAPNLPPTGLTTPPGSP

SEQ ID NO: 771 - protein, 33 aa, synthetic construct
  REGION 1..33: note = Synthetic
GTAEAASASGTTGRAGGAENLPPEGLTAPPGSP

SEQ ID NO: 772 - protein, 33 aa, synthetic construct
  REGION 1..33: note = Synthetic
GTAEAASASGTTSRAGTATNLTPEGLTAPPGSP

SEQ ID NO: 773 - protein, 33 aa, synthetic construct
  REGION 1..33: note = Synthetic
GTAEAASASGTTGRAGTATNLPPSGLTTPPGSP

SEQ ID NO: 774 - protein, 33 aa, synthetic construct
  REGION 1..33: note = Synthetic
GTAEAASASGTTARAGEAENLSPSGLTAPPGSP

SEQ ID NO: 775 - protein, 33 aa, synthetic construct
  REGION 1..33: note = Synthetic
GTAEAASASGTTGRAGGAGNLAPGGLTEPPGSP

SEQ ID NO: 776 - protein, 33 aa, synthetic construct
  REGION 1..33: note = Synthetic
GTAEAASASGTTGRAGTATNLPPEGLTGPPGSP

SEQ ID NO: 777 - protein, 33 aa, synthetic construct
  REGION 1..33: note = Synthetic
GTAEAASASGTTGRAGGAANLAPTGLTEPPGSP

SEQ ID NO: 778 - protein, 33 aa, synthetic construct
  REGION 1..33: note = Synthetic
GTAEAASASGTTGRAGTAENLAPSGLTTPPGSP

SEQ ID NO: 779 - protein, 33 aa, synthetic construct
  REGION 1..33: note = Synthetic
GTAEAASASGTTGRAGSATNLGPGGLTGPPGSP

SEQ ID NO: 780 - protein, 33 aa, synthetic construct
  REGION 1..33: note = Synthetic
GTAEAASASGTTARAGGAENLTPAGLTEPPGSP

SEQ ID NO: 781 - protein, 33 aa, synthetic construct
  REGION 1..33: note = Synthetic
GTAEAASASGTTARAGSAENLSPSGLTGPPGSP

SEQ ID NO: 782 - protein, 33 aa, synthetic construct
  REGION 1..33: note = Synthetic
GTAEAASASGTTARAGGAGNLAPEGLTTPPGSP

SEQ ID NO: 783 - protein, 33 aa, synthetic construct
  REGION 1..33: note = Synthetic
GTAEAASASGTTSRAGAAENLTPTGLTGPPGSP

SEQ ID NO: 784 - protein, 33 aa, synthetic construct
  REGION 1..33: note = Synthetic
GTAEAASASGTYGRTTTPGNEPPASLEAEPGSP

SEQ ID NO: 785 - protein, 33 aa, synthetic construct
  REGION 1..33: note = Synthetic
GTAEAASASGTYSRGESGPNEPPPGLTGPPGSP

SEQ ID NO: 786 - protein, 33 aa, synthetic construct
  REGION 1..33: note = Synthetic
GTAEAASASGAWGRTGASENETPAPLGGEPGSP

SEQ ID NO: 787 - protein, 33 aa, synthetic construct
  REGION 1..33: note = Synthetic
GTAEAASASGRWGRAETTPNTPPEGLETEPGSP

SEQ ID NO: 788 - protein, 33 aa, synthetic construct
  REGION 1..33: note = Synthetic
GTAEAASASGESGRAANHTGAEPPELGAGPGSP

SEQ ID NO: 789 - protein, 33 aa, synthetic construct
  REGION 1..33: note = Synthetic
GTAEAASASGTTGRAGEAANLTPAGLTESPGSP

SEQ ID NO: 790 - protein, 33 aa, synthetic construct
  REGION 1..33: note = Synthetic
GTAEAASASGTTGRAGEAANLTPAALTESPGSP

SEQ ID NO: 791 - protein, 33 aa, synthetic construct
  REGION 1..33: note = Synthetic
GTAEAASASGTTGRAGEAANLTPAPLTESPGSP

SEQ ID NO: 792 - protein, 33 aa, synthetic construct
  REGION 1..33: note = Synthetic
GTAEAASASGTTGRAGEAANLTPEPLTESPGSP

SEQ ID NO: 793 - protein, 33 aa, synthetic construct
  REGION 1..33: note = Synthetic
GTAEAASASGTTGRAGEAANLTPAGLTGAPGSP

SEQ ID NO: 794 - protein, 33 aa, synthetic construct
  REGION 1..33: note = Synthetic
GTAEAASASGTTGRAGEAANLTPEGLTGAPGSP

SEQ ID NO: 795 - protein, 33 aa, synthetic construct
  REGION 1..33: note = Synthetic
GTAEAASASGTTGRAGEAANLTPEPLTGAPGSP

SEQ ID NO: 796 - protein, 33 aa, synthetic construct
  REGION 1..33: note = Synthetic
GTAEAASASGTTGRAGEAANLTPAGLTEAPGSP

SEQ ID NO: 797 - protein, 33 aa, synthetic construct
  REGION 1..33: note = Synthetic
GTAEAASASGTTGRAGEAANLTPEGLTEAPGSP

SEQ ID NO: 798 - protein, 33 aa, synthetic construct
  REGION 1..33: note = Synthetic
GTAEAASASGTTGRAGEAANLTPAPLTEAPGSP

SEQ ID NO: 799 - protein, 33 aa, synthetic construct
  REGION 1..33: note = Synthetic
GTAEAASASGTTGRAGEAANLTPEPLTEAPGSP

SEQ ID NO: 800 - protein, 33 aa, synthetic construct
  REGION 1..33: note = Synthetic
GTAEAASASGTTGRAGEAANLTPEPLTGPPGSP

SEQ ID NO: 801 - protein, 33 aa, synthetic construct
  REGION 1..33: note = Synthetic
GTAEAASASGTTGRAGEAANLTPAGLTGGPGSP

SEQ ID NO: 802 - protein, 33 aa, synthetic construct
  REGION 1..33: note = Synthetic
GTAEAASASGTTGRAGEAANLTPEGLTGGPGSP

SEQ ID NO: 803 - protein, 33 aa, synthetic construct
  REGION 1..33: note = Synthetic
GTAEAASASGTTGRAGEAANLTPEALTGGPGSP

SEQ ID NO: 804 - protein, 33 aa, synthetic construct
  REGION 1..33: note = Synthetic
GTAEAASASGTTGRAGEAANLTPEPLTGGPGSP

SEQ ID NO: 805 - protein, 33 aa, synthetic construct
  REGION 1..33: note = Synthetic
GTAEAASASGTTGRAGEAANLTPAGLTEGPGSP

SEQ ID NO: 806 - protein, 33 aa, synthetic construct
  REGION 1..33: note = Synthetic
GTAEAASASGTTGRAGEAANLTPEGLTEGPGSP

SEQ ID NO: 807 - protein, 33 aa, synthetic construct
  REGION 1..33: note = Synthetic
GTAEAASASGTTGRAGEAANLTPAPLTEGPGSP

SEQ ID NO: 808 - protein, 33 aa, synthetic construct
  REGION 1..33: note = Synthetic
GTAEAASASGTTGRAGEAANLTPEPLTEGPGSP

SEQ ID NO: 809 - protein, 33 aa, synthetic construct; identical to SEQ ID NO: 732
  REGION 1..33: note = Synthetic

SEQ ID NO: 810 - protein, 30 aa, synthetic construct
  REGION 1..30: note = Synthetic
GTAEAASASGEAGRSAEATSAGATGPPGSP

SEQ ID NO: 811 - protein, 30 aa, synthetic construct
  REGION 1..30: note = Synthetic
GTAEAASASGSASGTYSRGESGPGSPPGSP

SEQ ID NO: 812 - protein, 30 aa, synthetic construct
  REGION 1..30: note = Synthetic
GTAEAASASGSASGEAGRTDTHPGSPPGSP

SEQ ID NO: 813 - protein, 30 aa, synthetic construct
  REGION 1..30: note = Synthetic
GTAEAASASGSASGEPGRAAEHPGSPPGSP

SEQ ID NO: 814 - protein, 30 aa, synthetic construct
  REGION 1..30: note = Synthetic
GTAEAASASGSPAGESSRGTTIAGSPPGSP

SEQ ID NO: 815 - protein, 33 aa, synthetic construct; identical to SEQ ID NO: 734
  REGION 1..33: note = Synthetic

SEQ ID NO: 816 - protein, 30 aa, synthetic construct
  REGION 1..30: note = Synthetic
GTAEAASASGEAGESAGATPAGLTGPPGSP

SEQ ID NO: 817 - protein, 30 aa, synthetic construct
  REGION 1..30: note = Synthetic
GTAEAASASGSASGAPLELEAGPGSPPGSP

SEQ ID NO: 818 - protein, 30 aa, synthetic construct
  REGION 1..30: note = Synthetic
GTAEAASASGSASGEPPELGAGPGSPPGSP

SEQ ID NO: 819 - protein, 30 aa, synthetic construct
  REGION 1..30: note = Synthetic
GTAEAASASGSASGEPSGLTEGPGSPPGSP

SEQ ID NO: 820 - protein, 30 aa, synthetic construct
  REGION 1..30: note = Synthetic
GTAEAASASGSASGTPAPLTEPPGSPPGSP

SEQ ID NO: 821 - protein, 30 aa, synthetic construct
  REGION 1..30: note = Synthetic
GTAEAASASGSASGTPAELTEPPGSPPGSP

SEQ ID NO: 822 - protein, 30 aa, synthetic construct
  REGION 1..30: note = Synthetic
GTAEAASASGSASGPPPGLTGPPGSPPGSP

SEQ ID NO: 823 - protein, 30 aa, synthetic construct
  REGION 1..30: note = Synthetic
GTAEAASASGSASGTPAPLGGEPGSPPGSP

SEQ ID NO: 824 - protein, 30 aa, synthetic construct
  REGION 1..30: note = Synthetic
GTAEAASASGSPAGAPEGLTGPAGSPPGSP

SEQ ID NO: 825 - protein, 30 aa, synthetic construct
  REGION 1..30: note = Synthetic
GTAEAASASGSPAGPPEGLETEAGSPPGSP

SEQ ID NO: 826 - protein, 30 aa, synthetic construct
  REGION 1..30: note = Synthetic
GTAEAASASGSPTSGQGGLTGPGSEPPGSP

SEQ ID NO: 827 - protein, 30 aa, synthetic construct
  REGION 1..30: note = Synthetic
GTAEAASASGSESAPPEGLETESTEPPGSP

SEQ ID NO: 828 - protein, 30 aa, synthetic construct
  REGION 1..30: note = Synthetic
GTAEAASASGSEGSEPLELGAASETPPGSP

SEQ ID NO: 829 - protein, 30 aa, synthetic construct
  REGION 1..30: note = Synthetic
GTAEAASASGSEGSGPAGLEAPSETPPGSP

SEQ ID NO: 830 - protein, 30 aa, synthetic construct
  REGION 1..30: note = Synthetic
GTAEAASASGSEPTPPASLEAEPGSPPGSP

SEQ ID NO: 831 - DNA, 4134 bp, synthetic construct
  misc_feature 1..4134: note = Synthetic
  source 1..4134: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgtgctgcagagcccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagagatcgtcctgacccaatcccccgggaccctcagcctgagcccaggcgagcgtgccactttgagctgtcgtgcatcacagagtgtgagttcctcattcctggcttggtaccagcaaaagcccggtcaggccccgagacttttgatttactatgcttccagccgcgctaccgggatcccagatagattttctgggagcggttctggtaccgatttcactctgaccatctctagactcgaaccagaagactttgcagtatattactgccaacagaccggtcggatccctccaactttcggacagggtaccaaggttgagatcaagggtgcaacgcctccggagactggtgctgaaactgagtccccgggcgagacgaccggtggctctgctgaatccgaaccaccgggcgaaggcgaggtccagctgttggagagcggcggtggactcgtgcagccgggcggttcacttcgtctcagttgtgctgcctcaggcttcacctttagctcattctcaatgagttgggtgagacaggcgcccggcaagggccttgagtgggttagttccatttccggctccagcggcactacctactatgccgactcagtcaaaggtagatttaccatctcccgcgataactctaagaacaccctgtacctgcagatgaactccctcagggcagaggataccgccgtgtactattgcgcgaagcccttcccatacttcgactactggggtcagggcaccctggtcactgtcagttccggcacagccgaggccgctagcgccagcggcgaggccggccggagcgccaaccacacccccgccggcctgaccggccctggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccaggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcacca

SEQ ID NO: 832 - DNA, 3609 bp, synthetic construct
  misc_feature 1..3609: note = Synthetic
  source 1..3609: mol_type = other DNA
atgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcacagccgaggccgctagcgccagcggcgaggccggccggagcgccaaccacacccccgccggcctgaccggccctggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccaggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 833 - DNA, 2562 bp, synthetic construct
  misc_feature 1..2562: note = Synthetic
  source 1..2562: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagaggccggccggagcgccaaccacacccccgccggcctgaccggccctggcacagccgaggccgctagcgccagcggcatgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccacat

SEQ ID NO: 834 - DNA, 3087 bp, synthetic construct
  misc_feature 1..3087: note = Synthetic
  source 1..3087: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaggccggccggagcgccaaccacacccccgccggcctgaccggccctggcacagccgaggccgctagcgccagcggcatgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 835 - DNA, 2823 bp, synthetic construct
  misc_feature 1..2823: note = Synthetic
  source 1..2823: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcatgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 836 - DNA, 3687 bp, synthetic construct
  misc_feature 1..3687: note = Synthetic
  source 1..3687: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccagaggccggccggagcgccaaccacacccccgccggcctgaccggccctggcacagccgaggccgctagcgccagcggcatgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcacagccgaggccgctagcgccagcggcgaggccggccggagcgccaaccacacccccgccggcctgaccggccctggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 837 - DNA, 3003 bp, synthetic construct
  misc_feature 1..3003: note = Synthetic
  source 1..3003: mol_type = other DNA
gcaagctccgccacccccgagtctggaccaggcaccagcacagagccttctgagggaagcgccccaggcacaagcgagtccgccacccctgagtccggaccaggatctggaccagccacctctgagagcgccacacctggcacctccgagtctgccacacctgagagcggaccaggatccgagccagccaccagcggctccgagacaccaggcacctctgaaagcgccactcctgagtccggaccaggcacctctacagagccttccgagggatctgccccaggaagcccagcaggcagcccaacctccacagaggagggcacatccgagtctgccactcctgagtctggacctggaagcgagccagccacaagcggaagcgaaacaccaggcacctctgagagcgccacgcctgagtccggacctggatctccagccggctctcctaccagcacagaggagggatccccagcaggatcccctacctctacagaggagggcaccagcacagagccaagcgagggatccgcccctggcacatccgaatctgccaccccagagtccggacctggcacaagcgaatccgccacccctgagagcggaccaggcacatctgagagcgccaccccagagagcggacctggatccgagccagccacatccggatctgagaccccaggatccgagcctgccacaagcggatccgagaccccaggaagccctgcaggatctcccaccagcaccgaagaaggcaccagcaccgagcccagcgaaggatctgcccctggcaccagcaccgagcctagcgagggatccgcccccggctccgagccagccacctctggaagtgaaacaccaggcacctccgaatctgccacaccagaggcaggccggtccgccaaccacaccccagcaggactgacaggaccaggcaccagcgaatccgccactccagagagcatgtgggagctggagaaggacgtgtacgtggtggaggtggactggacaccagatgcccccggcgagaccgtgaatctgacatgcgacacccccgaggaggacgatatcacctggacatccgatcagagacacggcgtgatcggctctggcaagaccctgacaatcaccgtgaaggagttcctggatgccggccagtacacatgtcacaagggcggcgagaccctgtctcacagccacctgctgctgcacaagaaggagaacggcatctggtccacagagatcctgaagaacttcaagaataagacctttctgaagtgcgaggccccaaattatagcggccggttcacctgttcctggctggtgcagagaaacatggacctgaagtttaatatcaagtctagctcctctagcccagatagcagggcagtgacatgcggaatggcatccctgtctgccgagaaggtgaccctggaccagagagattacgagaagtatagcgtgtcctgccaggaggacgtgacatgtcctaccgccgaggagaccctgccaatcgagctggccctggaggccaggcagcagaacaagtacgagaattattctaccagcttctttatccgcgacatcatcaagccagatccccctaagaacctgcagatgaagcccctgaagaatagccaggtcgaggtgtcctgggagtaccctgactcctggtctaccccacactcttatttcagcctgaagttctttgtgaggatccagcgcaagaaggagaagatgaaggagaccgaggagggctgcaaccagaagggcgcctttctggtggagaagacatccaccgaggtgcagtgcaagggaggaaacgtgtgcgtgcaggcacaggataggtactataattcctcttgtagcaagtgggcctgcgtgccctgtcgggtgagatctggcacagctactccagaaagcggaccaggagaggcaggccgcagcgccaatcatactcctgccggactgacaggacctgcaactcctgagtctggacccggcagccctgcaggatcccccacatctaccgaagaaggatccccagcaggaagccctacatccaccgaggagggaagcccagcaggatctcccacaagcaccgaggagggcacaagcgagtccgccacgcctgagtctggaccaggcacaagcaccgagccatccgagggatctgcccctggcacatctgaaagcgccactcccgaaagcggacctggatctgagccagccacctccggatctgagacaccaggcaccagcgagtccgccacacccgaatccggcccaggcagcgaacctgccacctctggaagcgagaccccaggcacctccgagtctgccacgcccgaatccggacctggcacatctaccgaaccttccgaaggatccgcccctggcagcccagcaggatctcctacaagcactgaagagggcacaagcgagtccgccactccagagtctggaccaggaagcgagcctgccacctctggcagcgagacccccggcacctccgagtctgccacccctgaatctggccctggatctccagccgggtcccccacatctaccgaggaaggctccccagcaggaagccccacatccactgaagaaggcacaagcactgaaccatccgaaggcagcgcccctggcacaagcgagtccgccacaccagagtcaggccctggcacatctgagagcgccacgccagagagcggacctggcacatccccatctgccactcctgagagtggccccgggtctgaaccagccacaagcggcagcgaaactcctggctccgagcctgccacatctgggtccgaaactcctggctccccagccggcagccccacatctactgaggagggcacaagcactgaaccctccgagggatccgccccaggcacatctaccgagccctccgaaggaagcgccccaggaagcgaacctgccacctccggctccgaaacccctggcaccagcgaatccgccggagagcctgaggcc

SEQ ID NO: 838 - DNA, 2211 bp, synthetic construct
  misc_feature 1..2211: note = Synthetic
  source 1..2211: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtgaacctgaggcc

SEQ ID NO: 839 - DNA, 2739 bp, synthetic construct
  misc_feature 1..2739: note = Synthetic
  source 1..2739: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgaggccggccggagcgccaaccacacccccgccggcctgaccggccctgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagaacctgaggcc

SEQ ID NO: 840 - DNA, 2214 bp, synthetic construct
  misc_feature 1..2214: note = Synthetic
  source 1..2214: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtgaacctgaggcctaa

SEQ ID NO: 841 - DNA, 1524 bp, synthetic construct
  misc_feature 1..1524: note = Synthetic
  source 1..1524: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgcctaa

SEQ ID NO: 842 - DNA, 2508 bp, synthetic construct
  misc_feature 1..2508: note = Synthetic
  source 1..2508: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgtgctgcagagcccaggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 843 - DNA, 2463 bp, synthetic construct
  misc_feature 1..2463: note = Synthetic
  source 1..2463: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgaggccggccggagcgccaaccacacccccgccggcctgaccggccctggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 844 - DNA, 3327 bp, synthetic construct
  misc_feature 1..3327: note = Synthetic
  source 1..3327: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccagaggccggccggagcgccaaccacacccccgccggcctgaccggccctggcacagccgaggccgctagcgccagcggcagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgaggccggccggagcgccaaccacacccccgccggcctgaccggccctggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 845 - DNA, 2163 bp, synthetic construct
  misc_feature 1..2163: note = Synthetic
  source 1..2163: mol_type = other DNA
gcaagctccgccacccctgagtctggaccaggcaccagcacagagccttccgagggatctgccccaggcaccagcgagtccgccacaccagagtccggacctggatctggaccaggcacctctgagagcgccaccccaggcacatccgagtctgccaccccagagagcggacctggatccgagccagccacaagcggatccgagaccccaggcacatctgaaagcgccactccagagtccggacctggcacctctacagagcctagcgagggatccgcccctggaagcccagccggctctcctaccagcacagaggagggcacctccgagtctgccacaccagagtctggaccaggaagcgagcctgccaccagcggcagcgaaactccaggcacatctgagagcgccacccctgagtccggaccaggatctcctgcaggatcccctacctctacagaggagggaagcccagcaggaagccccacctccaccgaagagggcacctccacagagccatctgagggaagcgcccctggcacctccgaatctgccacacctgagtccggacccggcaccagcgaatccgccacccccgagtctggacctggcacctctgaaagcgccacaccagagagcggaccaggatccgagcctgccacctccggatctgagacaccaggaagcgagccagccaccagcggatccgagacaccaggctcccccgccggctcccccacctctacagaggagggcaccagcaccgaaccttccgagggatccgcccccggcaccagcaccgagccttccgaaggaagcgccccaggctccgagccagccacctctggaagtgaaactcctggcacatccgaatctgccaccccagaggcaggcaggtccgccaaccacacaccagcaggactgaccggaccaggcacaagcgagtccgccaccccagagagccgcgtgatccccgtgtccggacctgcaaggtgcctgtctcagagcagaaatctgctgaagaccacagacgatatggtgaagaccgcccgggagaagctgaagcactacagctgtacagccgaggacatcgatcacgaggacatcaccagagatcagaccagcacactgaagacatgcctgcccctggagctgcacaagaacgagtcctgtctggccacccgggagacatctagcaccacaagaggctcttgcctgccccctcagaagaccagcctgatgatgaccctgtgcctgggcagcatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccataccgcgtgaagatgaagctgtgcatcctgctgcacgccttttccaccagggtggtgacaatcaaccgcgtgatgggctatctgtcctctgccggcactgctacaccagagtccggaccaggagaggcaggccggtctgccaatcacacccctgccggactgaccggacctgcaacaccagagtctggacctggctctgagccagccacctccggctccgagacccctggcacaagcgaatccgccaccccagaaagcggccctggctccccagccggcagccctacctctaccgaagaaggcagcccagcaggaagccctacctccaccgaggaaggcacctccacagagccttctgagggaagcgcccccggcacctctgaaagcgccacgccagaatccggaccaggcacctccgagtctgccacgcctgagtccggaccaggcaccagcgcctccgccacacccgagagcggcccagggagcgaaccagccacctctggaagcgaaacccctggcagtgaaccagccacctccggctctgagacaccaggatccccagccggctcacctacctctaccgaggagggcaccagcactgaacctagtgagggatccgccccaggcacctctaccgaacctagcgaaggcagcgcccctggctcagagccagccaccagcggcagcgagactcctggcacatctgaaagcgccggc

SEQ ID NO: 846 - DNA, 4233 bp, synthetic construct
  misc_feature 1..4233: note = Synthetic
  source 1..4233: mol_type = other DNA
atgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcggcggcggctctggcggcggcggctccggcggcggcggctccagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgccggcacagccgaggccgctagcgccagcggcgaggccggccggagcgccaaccacacccccgccggcctgaccggccctggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccaggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 847 - DNA, 4233 bp, synthetic construct
  misc_feature 1..4233: note = Synthetic
  source 1..4233: mol_type = other DNA
ggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaggtacaagcaccgaacctagcgagggcagcgctcccggcagccctgccggcagcccaacctcaactgaggagggcaccagtactgagcccagcgagggatcagcacctggcaccagcaccgaacctagcgaggggagcgcccctgggactagcgagtcagctacaccagagagcgggcctggaacttctaccgaacccagtgagggatccgctccaggcacctccgaatccgcaacccccgaatccggacctggctcagagcccgccaccagcgggagcgaaacccctggcacatccaccgagcctagcgaagggtccgcacccggcaccagtacagagcctagcgagggatcagcacctggcaccagtgaatctgctacaccagagagcggccctggaacctccgagtccgctacccccgagagcgggccaggttctcctgctggctcccccacctcaacagaagaggggacaagcgaaagcgctacgcctgagagtggccctggctctgagccagccacctccggctctgaaacccctggcactagtgagtctgccacgcctgagtccggacccgggacctctactgagccctcggaggggagcgctcctggcacgagtacagaaccttccgaaggaagtgcaccgggcacaagcaccgagccttccgaaggctctgctcccggaacctctaccgaaccctctgaagggtctgcacccggcacgagcaccgaacccagcgaagggtcagcgcctgggacctcaacagagccctcggaaggatcagcgcctggaagccctgcagggagtccaacttccacggaagaaggaacgtctacagagccatcagaggggtccgcaccaggtaccagcgaatccgctactcccgaatctggccctgggtccgaacctgccacctccggctctgaaactccagggacctccgaatctgccacacccgagagcggccctggctccgagcccgcaacatctggcagcgagacacctggcacctccgagagcgcaacacccgagagcggccctggcaccagcaccgagccatccgagggatccgccccaggcacttctgagtcagccacacccgaaagcggaccaggatcacccgctggctcccccaccagtaccgaggaggggtcccccgctggaagtccaacaagcactgaggaagggtcccctgccggctcccccacaagtaccgaagagggcacaagtgagagcgccactcccgagtccgggcctggcaccagcacagagccttccgaggggtccgcaccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagaggccggccggagcgccaaccacacccccgccggcctgaccggccctggcacagccgaggccgctagcgccagcggcatgtgggagctggagaaggacgtgtacgtggtggaggtggactggacaccagatgcccccggcgagaccgtgaacctgacatgcgacacccccgaggaggacgatatcacctggacatctgatcagaggcacggcgtgatcggaagcggcaagaccctgacaatcaccgtgaaggagttcctggatgccggccagtacacatgtcacaagggcggcgagaccctgtcccactctcacctgctgctgcacaagaaggagaacggcatctggtccacagagatcctgaagaacttcaagaataagacctttctgaagtgcgaggcccctaattatagcggccggttcacctgttcctggctggtgcagagaaacatggacctgaagtttaatatcaagagctcctctagctccccagatagccgggcagtgacatgcggaatggccagcctgtccgccgagaaggtgaccctggaccagagagattacgagaagtattctgtgagctgccaggaggacgtgacatgtcccaccgccgaggagacactgcctatcgagctggccctggaggccaggcagcagaacaagtacgagaattattccacctctttctttatccgcgacatcatcaagccagatccccctaagaacctgcagatgaagcccctgaagaattcccaggtcgaggtgtcttgggagtaccctgacagctggtccacaccacactcttatttcagcctgaagttctttgtgaggatccagcgcaagaaggagaagatgaaggagaccgaggagggctgcaatcagaagggcgcctttctggtggagaagacatccaccgaggtgcagtgcaagggaggaaacgtgtgcgtgcaggcacaggatcggtactataattctagctgttccaagtgggcctgcgtgccttgtcgggtgagatctggcggcggcggctctggcggcggcggctccggcggcggcggctccagagtgatccccgtgagcggaccagcaaggtgcctgtcccagagccggaacctgctgaagaccacagacgatatggtgaagaccgcccgggagaagctgaagcactactcttgtacagccgaggacatcgatcacgaggacatcacccgggatcagacctctacactgaagacatgcctgcccctggagctgcacaagaacgagagctgtctggccacccgggagacaagctccaccacaagaggcagctgcctgccccctcagaagacctccctgatgatgaccctgtgcctgggctctatctacgaggacctgaagatgtatcagaccgagttccaggccatcaatgccgccctgcagaaccacaatcaccagcagatcatcctggacaagggcatgctggtggccatcgatgagctgatgcagagcctgaaccacaatggcgagaccctgaggcagaagccaccagtgggagaggcagatccttacagggtgaagatgaagctgtgcatcctgctgcacgccttttccaccagggtggtgacaatcaatcgcgtgatgggctatctgtctagcgcc

SEQ ID NO: 848 - DNA, 5169 bp, synthetic construct
  misc_feature 1..5169: note = Synthetic
  source 1..5169: mol_type = other DNA
gcaagctccgccaccccagagtccggacctggcacctctacagagccaagcgagggatccgccccaggcacaagcgagtccgccaccccagagtctggaccaggaagcggacctgccacctctgagagcgccacaccaggcacctccgagtctgccacaccagagtccggaccaggatctgagcctgccaccagcggatccgagacacctggcacctctgaaagcgccactccagagagcggaccaggcacctccaccgagccttctgagggaagcgccccaggaagccctgcaggatccccaacctctacagaggagggcacatccgagtctgccacccctgagagcggaccaggatccgagccagccacaagcggatccgagacaccaggcacctctgagagcgccacgcctgaatccggaccaggaagcccagcaggaagccccacctccacagaggagggatcccctgcaggatctccaaccagcacagaggagggcaccagcacagagccttccgagggctctgccccaggcacatccgaatctgccactcctgagtctggacctggcacaagcgaatccgccacccccgaaagcggaccaggcacatctgagagcgccacccctgagtctggcccaggatctgagccagccacatccggctctgagacccctggcagcgaacctgccacaagcggcagcgagacccctggaagcccagcaggctcccccacctccaccgaagaaggcaccagcacagagccatctgagggaagcgcccctggcaccagcaccgaaccatccgagggatctgccccaggatccgagcctgccacctctggcagtgaaacccctggcacctccgaatctgccacacccgaggcaggccggtccgccaaccacaccccagccggcctgacaggacctggcaccagcgaatccgccactccagagagcatgtgggagctggagaaggacgtgtacgtggtggaggtggactggacacccgatgcccctggcgagaccgtgaatctgacatgcgacacccctgaggaggacgatatcacctggacatccgatcagagacacggcgtgatcggctctggcaagaccctgacaatcaccgtgaaggagttcctggatgccggccagtacacatgtcacaagggcggcgagaccctgtctcacagccacctgctgctgcacaagaaggagaacggcatctggtccacagagatcctgaagaacttcaagaataagacctttctgaagtgcgaggcccccaattatagcggcaggttcacctgttcctggctggtgcagcgcaacatggacctgaagtttaatatcaagtctagctcctctagccctgatagcagggcagtgacatgcggaatggcatccctgtctgccgagaaggtgaccctggaccagagagattacgagaagtatagcgtgtcctgccaggaggacgtgacatgtcctaccgccgaggagaccctgccaatcgagctggccctggaggccaggcagcagaacaagtacgagaattattctaccagcttctttatccgcgacatcatcaagccagatccccctaagaacctgcagatgaagcccctgaagaattcccaggtggaggtgagctgggagtacccagactcctggtctaccccccacagctatttctccctgaagttctttgtgaggatccagcgcaagaaggagaagatgaaggagaccgaggagggctgcaaccagaagggcgcctttctggtggagaagacatccaccgaggtgcagtgcaagggaggaaacgtgtgcgtgcaggcacaggataggtactataattcctcttgtagcaagtgggcatgcgtgccatgtcgggtgagatccggcacagctactcctgaatctggaccaggagaggcaggccgcagcgccaaccacacccctgcaggactgacaggaccagcaaccccagagagcggacctggatccccagccggctctccaacaagcaccgaagaaggatctccagcaggatccccaacatctaccgaggagggctccccagcaggaagccctacatccaccgaggagggcacaagcgagtccgccacgccagagtccggaccaggcacatctaccgaaccaagcgaaggaagcgcccctggcacatctgaaagcgccactcccgaaagcggaccaggaagcgagccagccacctccggatctgagacaccaggcaccagcgagtccgccacacctgagtctgggcctggctctgagccagccacctctggaagtgaaacccccggcacctccgagtctgccacgcctgagagcggaccaggcacatccaccgagcctagcgaaggctctgcccctggcagccctgccggctcccctacatccactgaggagggcacaagcgagtccgccactcctgaaagcggacctggatccgaacctgccacctctggaagtgagacccctggcacctccgagtctgccacccccgaatctggccctggctccccagcaggctctcccacaagcaccgaggagggatccccagcaggatcccctacatctactgaagagggcacaagcaccgaacctagcgagggatccgcccctggcacaagcgagtccgccacacccgaatctggccccggcacatctgaaagcgccacgccagaatccggcccaggcacatccccatctgccacccctgagtctgggcctgggtctgaacctgccacaagcgggagcgagacccctggcagcgagccagccacatctggatccgaaactccaggctccccagcaggatcccccacaagcactgaagaaggcacaagcaccgagcctagcgaggggtctgcccctggcacatctaccgagccctccgaaggctccgccccaggaagcgagcctgccacctccggctctgagacacctggcaccagcgagtccgccggagcctcctccgccactcctgaatccggacctggcacaagcactgaaccttccgaaggaagcgcccccggcacatctgagagcgccactccagaatccggaccaggatccggccccggcacctccgagtctgccactcccggcaccagcgaatccgccacgcctgagtccggccctgggagcgaacccgccacctctggaagcgaaaccccaggcacctccgaatctgccacgcctgagtctggcccaggcacatctactgaacctagcgaagggtctgcccctgggagccctgcaggcagccccacatccacagaagaaggcacaagcgaatccgccacacctgagtccggacctggatccgagcccgccacctctggctccgaaactcctggcacctccgagtctgccacgccggaatctggaccaggatctcctgccggatcccccacaagcacagaagaagggagccctgccggatcccctacatctacagaagagggcacaagcactgagccctccgaagggtccgcccccggcacaagcgagtccgccacgccggaaagtggccctggcacatctgagagcgccacacccgagtctgggccaggcacatccgagtctgccacgccagagtctggacctggaagtgaacccgccacaagcggctccgagactcctggcagcgagcctgccacatctggatccgagactcctggaagcccagcaggatcacccacaagcactgaggagggcacatccaccgagcccagcgagggatctgcccctggcacatccacagaaccttccgaaggatccgcccctggctccgaacctgccacctccgggagcgaaaccccaggcaccagcgaatccgccaccccagaggcaggccggagcgccaaccacacccccgctggactgaccggccctggcacctctgagagcgccaccccagagtctagagtgatccctgtgagcggaccagcaaggtgcctgtcccagtctagaaatctgctgaagaccacagacgatatggtgaagacagccagggagaagctgaagcactacagctgtaccgccgaggacatcgatcacgaggacatcacacgcgatcagacatccaccctgaagacctgcctgcccctggagctgcacaagaacgagagctgtctggccacacgggagacctctagcaccacaagaggcagctgcctgccaccccagaagacatccctgatgatgaccctgtgcctgggcagcatctacgaggacctgaagatgtatcagaccgagttccaggccatcaatgccgccctgcagaaccacaatcaccagcagatcatcctggacaagggcatgctggtggccatcgatgagctgatgcagtccctgaaccacaatggcgagaccctgaggcagaagcctccagtgggagaggccgatccctacagagtgaagatgaagctgtgcatcctgctgcacgcctttagcacaagggtggtgaccatcaaccgcgtgatgggctatctgtcctctgccggaacagcaacccctgaatctggacctggagaggcaggcaggagcgccaatcacaccccagccgggctgaccggcccagcaacccctgagtccggcccagggtccgagccagccaccagcggcagcgaaactccaggcacctctgagagcgccactcctgagtccgggccaggatccccagcaggatctcctacaagcactgaagaagggtctcccgccggcagcccaacatctactgaggaaggcacaagcactgaaccctccgaaggatccgcccccggcacatccgagtctgccactcctgagagcggacccggcacaagcgagtccgccacgcctgaaagtggaccaggcacatctgccagcgccactccagaaagcggccctggaagcgaacctgccacatccggctccgagacccccggctctgaaccagccacaagcggcagcgaaactcccggaagcccagcaggatctcccacaagcactgaagagggcacaagcacggagcctagcgaaggatctgcccccggcacaagcactgaacccagtgaaggatccgccccaggcagcgaaccagccacctctggaagcgagacccctggcacctccgagtctgccggagagcctgaggcctga

SEQ ID NO: 849 - protein, 1378 aa, synthetic construct
  REGION 1..1378: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTAEAASASGVLQSPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGATPPETGAETESPGETTGGSAESEPPGEGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGTAEAASASGEAGRSANHTPAGLTGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 850 - protein, 1203 aa, synthetic construct
  REGION 1..1203: note = Synthetic
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTAEAASASGEAGRSANHTPAGLTGPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 851 - protein, 853 aa, synthetic construct
  REGION 1..853: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPEAGRSANHTPAGLTGPGTAEAASASGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTAEAASASGEAGRSANHTPAGLTGPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 852 - protein, 1029 aa, synthetic construct
  REGION 1..1029: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEAGRSANHTPAGLTGPGTAEAASASGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTAEAASASGEAGRSANHTPAGLTGPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 853 - protein, 941 aa, synthetic construct
  REGION 1..941: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTAEAASASGEAGRSANHTPAGLTGPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 854 - protein, 1229 aa, synthetic construct
  REGION 1..1229: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPEAGRSANHTPAGLTGPGTAEAASASGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTAEAASASGEAGRSANHTPAGLTGPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 855 - protein, 1001 aa, synthetic construct
  REGION 1..1001: note = Synthetic
ASSATPESGPGTSTEPSEGSAPGTSESATPESGPGSGPATSESATPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPEAGRSANHTPAGLTGPGTSESATPESMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTATPESGPGEAGRSANHTPAGLTGPATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSPSATPESGPGSEPATSGSETPGSEPATSGSETPGSPAGSPTSTEEGTSTEPSEGSAPGTSTEPSEGSAPGSEPATSGSETPGTSESAGEPEA

SEQ ID NO: 856 - protein, 737 aa, synthetic construct
  REGION 1..737: note = Synthetic
EPEAGTAEAASASGGTAEAASASGEAGRSANHTPAGLTGPEAGRSANHTPAGLTGPRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGP

SEQ ID NO: 857 - protein, 913 aa, synthetic construct
  REGION 1..913: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTAEAASASGEAGRSANHTPAGLTGPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPEPEA

SEQ ID NO: 858 - protein, 737 aa, synthetic construct
  REGION 1..737: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTAEAASASGEAGRSANHTPAGLTGPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGEPEA

SEQ ID NO: 859 - protein, 507 aa, synthetic construct
  REGION 1..507: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 860 - protein, 836 aa, synthetic construct
  REGION 1..836: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTAEAASASGVLQSPGTAEAASASGEAGRSANHTPAGLTGPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 861 - protein, 821 aa, synthetic construct
  REGION 1..821: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTAEAASASGEAGRSANHTPAGLTGPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 862 - protein, 1109 aa, synthetic construct
  REGION 1..1109: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPEAGRSANHTPAGLTGPGTAEAASASGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTAEAASASGEAGRSANHTPAGLTGPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 863 - protein, 721 aa, synthetic construct
  REGION 1..721: note = Synthetic
ASSATPESGPGTSTEPSEGSAPGTSESATPESGPGSGPGTSESATPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPEAGRSANHTPAGLTGPGTSESATPESRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTATPESGPGEAGRSANHTPAGLTGPATPESGPGSEPATSGSETPGTSESATPESGPGSPAGSPTSTEEGSPAGSPTSTEEGTSTEPSEGSAPGTSESATPESGPGTSESATPESGPGTSASATPESGPGSEPATSGSETPGSEPATSGSETPGSPAGSPTSTEEGTSTEPSEGSAPGTSTEPSEGSAPGSEPATSGSETPGTSESAG

SEQ ID NO: 864 - protein, 1203 aa, synthetic construct; identical to SEQ ID NO: 850
  REGION 1..1203: note = Synthetic

SEQ ID NO: 865 - protein, 1411 aa, synthetic construct
  REGION 1..1411: note = Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPEAGRSANHTPAGLTGPGTAEAASASGMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 866 - protein, 1722 aa, synthetic construct
  REGION 1..1722: note = Synthetic
ASSATPESGPGTSTEPSEGSAPGTSESATPESGPGSGPATSESATPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPEAGRSANHTPAGLTGPGTSESATPESMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGTATPESGPGEAGRSANHTPAGLTGPATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSPSATPESGPGSEPATSGSETPGSEPATSGSETPGSPAGSPTSTEEGTSTEPSEGSAPGTSTEPSEGSAPGSEPATSGSETPGTSESAGASSATPESGPGTSTEPSEGSAPGTSESATPESGPGSGPGTSESATPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPEAGRSANHTPAGLTGPGTSESATPESRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGTATPESGPGEAGRSANHTPAGLTGPATPESGPGSEPATSGSETPGTSESATPESGPGSPAGSPTSTEEGSPAGSPTSTEEGTSTEPSEGSAPGTSESATPESGPGTSESATPESGPGTSASATPESGPGSEPATSGSETPGSEPATSGSETPGSPAGSPTSTEEGTSTEPSEGSAPGTSTEPSEGSAPGSEPATSGSETPGTSESAGEPEA

SEQ ID NO: 867 - protein, 8 aa, synthetic construct
  REGION 1..8: note = Synthetic
LEVLFQGP

SEQ ID NO: 868 - protein, 8 aa, synthetic construct
  REGION 1..8: note = Synthetic
LPKTGSES

SEQ ID NO: 869-875 - skipped (7 entries)

SEQ ID NO: 876 - protein, 8 aa, synthetic construct
  REGION 1..8: note = Synthetic
  VAR_SEQ 2: note = Xaa is P or A
  VAR_SEQ 3: note = Xaa is any amino acid
  VAR_SEQ 6: note = Xaa is any amino acid
  VAR_SEQ 8: note = Xaa is any amino acid
GXXGLXGX

SEQ ID NO: 877 - protein, 8 aa, synthetic construct
  VAR_SEQ 3: note = Xaa is any amino acid
  VAR_SEQ 6: note = Xaa is any amino acid
  VAR_SEQ 7: note = Xaa is G or A
  VAR_SEQ 8: note = Xaa is any amino acid
GPXGLXXX

SEQ ID NO: 878 - skipped

SEQ ID NO: 879 - protein, 7 aa, synthetic construct
  REGION 1..7: note = Synthetic
  VAR_SEQ 7: note = Xaa is G or S
ENLYGQX

SEQ ID NO: 880 - protein, 8 aa, synthetic construct
  REGION 1..8: note = Synthetic
  VAR_SEQ 3: note = Xaa is K, E, A or D
  VAR_SEQ 6: note = Xaa is any amino acid
  VAR_SEQ 7: note = Xaa is E, K or S
LPXTGXXS

SEQ ID NO: 881 - protein, 12 aa, synthetic construct
  REGION 1..12: note = Synthetic
GSGASEPTSTEP

SEQ ID NO: 882 - protein, 12 aa, synthetic construct
  REGION 1..12: note = Synthetic
GSEPATSGTEPS

SEQ ID NO: 883 - protein, 12 aa, synthetic construct
  REGION 1..12: note = Synthetic
GTSEPSTSEPGA

SEQ ID NO: 884 - protein, 12 aa, synthetic construct
  REGION 1..12: note = Synthetic
GTSTEPSEPGSA

SEQ ID NO: 885 - protein, 12 aa, synthetic construct
  REGION 1..12: note = Synthetic
GSTAGSETSTEA

SEQ ID NO: 886 - protein, 12 aa, synthetic construct
  REGION 1..12: note = Synthetic
GSETATSGSETA

SEQ ID NO: 887 - protein, 12 aa, synthetic construct
  REGION 1..12: note = Synthetic
GTSESATSESGA

SEQ ID NO: 888 - protein, 12 aa, synthetic construct
  REGION 1..12: note = Synthetic
GTSTEASEGSAS

SEQ ID NO: 889 - protein, 874 aa, synthetic construct
  REGION 1..874: note = Synthetic
PGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGFTABTSESATPESGPGSEPATSGPTESGSEPATSGSETPGSPAGSPTSTEEGTSTEPSEGSAPGTESTPSEGSAPGSEPATSGSETPGTSESATPESGPGTSTEPSEGSAPGEPEA

SEQ ID NO: 890 - protein, 870 aa, synthetic construct
  REGION 1..870: note = Synthetic
PGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGPTESGSEPATSGSETPGSPAGSPTSTEEGTSTEPSEGSAPGTSTEPSEGSAPGSEPATSGSETPGTSESATPESGPGTSTEPSEGSAPGEPEA

SEQ ID NO: 891 - protein, 870 aa, synthetic construct
  REGION 1..870: note = Synthetic
PGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTESTPSEGSAPGSEPATSGSETPGTSESATPESGPGTSTEPSEGSAPGEPEA

SEQ ID NO: 892 - protein, 294 aa, synthetic construct
  REGION 1..294: note = Synthetic
ASSPAGSPTSTESGTSESATPESGPGTETEPSEGSAPGTSESATPESGPGSEPATSGSETPGTSESATPESGPGSTPAESGSETPGTSESATPESGPGTSTEPSEGSAPGSPAGSPTSTEEGTSESATPESGPGESPATSGSTPEGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGGSAP

SEQ ID NO: 893 - protein, 294 aa, synthetic construct
  REGION 1..294: note = Synthetic
ASSPAGSPTSTESGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGESPATSGSTPEGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGGSAP

SEQ ID NO: 894 - protein, 294 aa, synthetic construct
  REGION 1..294: note = Synthetic
ASSPAGSPTSTESGTSESATPESGPGTSTEPSEGSAPGTSESATPESGPGSEPATSGSETPGTSESATPESGPGSTPAESGSETPGTSESATPESGPGTSTEPSEGSAPGSPAGSPTSTEEGTSESATPESGPGEEPATSGSTPEGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGGSAP

SEQ ID NO: 895 - protein, 294 aa, synthetic construct
  REGION 1..294: note = Synthetic
ASSPAGSPTSTESGTSESATPESGPGTSTEPSEGSAPGTSESATPESGPGSEPATSGSETPGTSESATPESGPGSTPAESGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGGSAP

SEQ ID NO: 896 - protein, 866 aa, synthetic construct
  REGION 1..866: note = Synthetic
PGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTESTPSEGSAPGSEPATSGSETPGTSESATPESGPGTSTEPSEGSAPG

SEQ ID NO: 897 - protein, 578 aa, synthetic construct
  REGION 1..578: note = Synthetic
PGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTESTPSEGSAPGSEPATSGSETPGTSESATPESGPGTSTEPSEGSAPG

SEQ ID NO: 898 - protein, 1152 aa, synthetic construct
  REGION 1..1152: note = Synthetic
SAGSPGSPAGSPTSTEEGTSESATPESGPGTSTEPSEGSAPGSPAGSPTSTEEGTSTEPSEGSAPGTSTEPSEGSAPGTSESATPESGPGSTPAESGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSTETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTESAS

SEQ ID NO: 899 - protein, 1153 aa, synthetic construct
  REGION 1..1153: note = Synthetic
SAGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSTETPGSPAGSPTSTEEGTSESATPESGPGTSTEPSEGSAPGTSTEPSEGSAPGSPAGSPTSTEEGTSTEPSEGSAPGTATESPEGSAPGTSESATPESGPGTSTEPSEGSAPGTSAESATPESGPGSEPATSGSETPGTSTEPSEGSAPGTSTEPSEGSAPGTSESATPESGPGTESAS

SEQ ID NO: 900 - protein, 864 aa, synthetic construct
  REGION 1..864: note = Synthetic
GSPAGSPTSTEEGTSESATPESGPGTSTEPSEGSAPGSPAGSPTSTEEGTSTEPSEGSAPGTSTEPSEGSAPATSESATPESGPGSEPATSGSETPGSEPATSGSETPGSPAGSPTSTEEGTSESASPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 901 - protein, 864 aa, synthetic construct
  REGION 1..864: note = Synthetic
GSPAGSPTSTEEGTSESATPESGPGTSTEPSEGSAPGSPAGSPTSTEEGTSTEPSEGSAPGTSTEPSEGSAPGTSESATPESGPGSESATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 902 - protein, 292 aa, synthetic construct
  REGION 1..292: note = Synthetic
SPAGSPTSTESGTSESATPESGPGTSTEPSEGSAPGTSESATPESGPGSEPATSGSETPGTSESATPESGPGSTPAESGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGGSAP

SEQ ID NO: 903 - protein, 582 aa, synthetic construct
  REGION 1..582: note = Synthetic
PGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTESTPSEGSAPGSEPATSGSETPGTSESATPESGPGTSTEPSEGSAPGEPEA

SEQ ID NO: 904 - protein, 576 aa, synthetic construct
  REGION 1..576: note = Synthetic
TPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSESATSGSETPGSPAGSPTSTEEGTSTEPSEGSAPGTSTEPSEGSAPGSEPATSGSETPGTSESA

SEQ ID NO: 905 - protein, 576 aa, synthetic construct
  REGION 1..576: note = Synthetic
GTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESASPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGP

SEQ ID NO: 906 - protein, 576 aa, synthetic construct
  REGION 1..576: note = Synthetic
GSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSTETGTSESATPESGPGSEPATSGSETPGTSESATPESGPGTSTEPSEGSAPGSPAGSPTSTEEGTSESATPESGPGSEPATS

SEQ ID NO: 907 - protein, 576 aa, synthetic construct
  REGION 1..576: note = Synthetic
EGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESASPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESAT

SEQ ID NO: 908 - protein, 294 aa, synthetic construct; identical to SEQ ID NO: 895
  REGION 1..294: note = Synthetic

SEQ ID NO: 909 - protein, 5 aa, synthetic construct
  REGION 1..5: note = Sortase A
LPETG

(19) United States
(12) Patent Application Publication
(10) Pub. No.: US 2023/0265146 A1
(43) Pub. Date: Aug. 24, 2023

(54) CYTOKINE CONJUGATES

(71) Applicant: AMUNIX PHARMACEUTICALS, INC., South San Francisco, CA (US)

(72) Inventors: Volker SCHELLENBERGER, Palo Alto, CA (US); Eric JOHANSEN, Oakland, CA (US); Angela HENKENSIEFKEN, San Jose, CA (US); Bryan IRVING, Woodside, CA (US); Tracy YOUNG, Belmont, CA (US); Vibha CHAUHAN, Saratoga, CA (US); Valentine YEUNG, Belmont, CA (US)

(21) Appl. No.: 18/073,935

(22) Filed: Dec. 2, 2022

Related U.S. Application Data
(63) Continuation of application No. PCT/US2021/038909, filed on Jun. 24, 2021.
(60) Provisional application No. 63/197,944, filed on Jun. 7, 2021.
(60) Provisional application No. 63/197,875, filed on Jun. 7, 2021.
(60) Provisional application No. 63/044,335, filed on Jun. 25, 2020.

(51) Int. Cl. C07K 14/54; C07K 14/00; A61P 35/00

(52) U.S. Cl. CPC C07K 14/5434; C07K 14/001; A61P 35/00; C07K 2319/33

(57) ABSTRACT

The present invention relates to compositions comprising biologically active proteins, such as cytokines, linked to extended recombinant polypeptide (XTEN), isolated nucleic acids encoding the compositions and vectors and host cells containing the same, and methods of using such compositions in treatment of related disorders and conditions.

### RELATED APPLICATIONS

This application is a continuation of International Patent Application No. PCT/US2021/038909, filed Jun. 24, 2021, which claims priority to U.S. Provisional Patent Application Nos. 63/044,335, filed Jun. 25, 2020; 63/197,875, filed Jun. 7, 2021; and 63/197,944 filed Jun. 7, 2021, the entire disclosures of which are hereby incorporated herein by reference.

### BACKGROUND

Cytokines can be used to treat a variety of diseases or conditions, such as cancer, inflammatory conditions, autoimmune conditions, rheumatoid arthritis, multiple sclerosis, myasthenia gravis, systemic lupus erythematosus, Alzheimer's disease, Schizophrenia, viral infections, (e.g., chronic hepatitis C, AIDS), allergic asthma, retinal neurodegenerative processes, metabolic disorder, insulin resistance, and diabetic cardiomyopathy. However, the therapeutic utility of cytokines can be limited due to the cellular toxicity, short half-life, need for repetitive or frequent dosing, and the potential to elicit undesired immune response in the patients.

Most cytokine products in the clinical setting are extremely potent. Interleukins, such as IL-2 and IL-12, and IFN-α are cytokines, produced primarily by cells of the immune system to signal and organize the immune response. In cancer, cytokines facilitate the ability of the immune system to recognize tumor cells as abnormal and harmful to the host. Cytokines further increase the proliferation of, enhance the survival of, and direct a variety of immune cell types to infiltrate the TME and promote potent anti-tumor immune responses resulting in tumor cell killing and tumor clearance. This limits the practical applications of cytokines in a therapeutic setting, particularly in anti-cancer indications.

Interleukin-12 (IL12) in particular, has been recognized as having potential to be an ideal payload for tumor immunotherapy. It can activate both the innate and the adaptive components of the immune system. IL12 stimulates the production of IFN-γ and activates NK cells, as well as CD8+ and CD4+ T cells. In addition, this cytokine also induces antiangiogenic chemokines, remodeling of the tumor extracellular matrix and stimulation of MHC class I molecules expression, making it an extremely attractive anticancer candidate. However, while researchers have shown encouraging preclinical data, the severe toxicity profile of this cytokine has prevented dose escalation and significantly curbed clinical potential as an anticancer agent. Although multiple clinical trials have been on-going since the first human clinical trial of IL12 in 1996, an FDA-approved IL12 product remains elusive.

This presents a significant unmet need for new strategies that can overcome therapeutic index challenges for use of cytokines as anticancer agents. If the potency of cytokines like IL12 could be safely harnessed and the toxicity challenges could be controlled, these agents could serve as powerful therapeutics for potential use against a broad spectrum of cancers.

### SUMMARY

The present disclosure includes cytokine-related compositions and related methods that may address one or more drawback, or may provide one or more advantages. In one aspect, disclosed herein is a fusion protein comprising:
(a) an extended recombinant polypeptide (XTEN) characterized in that:
   i. it comprises at least 12 amino acids;
   ii. at least 90%, 91%, 92%, 93%, 94%, 95%, 96%, 97%, 98%, 99%, or 100% of the amino acid residues of the XTEN sequence are selected from glycine (G), alanine (A), serine (S), threonine (T), glutamate (E) and proline (P); and
   iii. it has 4-6 different amino acids selected from G, A, S, T, E and P; and
(b) a cytokine linked to the XTEN.

In some embodiments, the fusion protein further comprises a release segment, wherein the release segment (RS) has at least 88%, at least 94%, or 100% sequence identity to a sequence selected from the sequences set forth in Tables 6-7. In some embodiments, the fusion protein has a structural arrangement, from N- to C-terminus of XTEN-RS-cytokine or cytokine-RS-XTEN.

In some embodiments, the cytokine is selected from a group consisting of interleukins, chemokines, interferons, tumor necrosis factors, colony-stimulating factors, or TGF-Beta superfamily members. In some embodiments, the cytokine is an interleukin selected from the group consisting of IL1, IL2, IL3, IL4, IL5, IL6, IL7, IL8, IL9, IL10, IL11, IL12, IL13, IL14, IL15, IL16, and IL17. In some embodiments, the cytokine has at least 90% sequence identity to a sequence selected from Table 3 or Table A. In some embodiments, the cytokine is IL-12 or an IL-12 variant. In some embodiments, the cytokine comprises a first cytokine fragment (Cy1) and a second cytokine fragment (Cy2). In some embodiments, one of the Cy1 and the Cy2 comprises an amino acid sequence having at least 70%, 75%, 80%, 85%, 90%, 91%, 92%, 93%, 94%, 95%, 96%, 97%, 98%, 99%, or 100% sequence identity to an interleukin-12 subunit beta. In some embodiments, the other one of the Cy1 and the Cy2 comprises an amino acid sequence having at least 70%, 75%, 80%, 85%, 90%, 91%, 92%, 93%, 94%, 95%, 96%, 97%, 98%, 99%, or 100% sequence identity to an interleukin-12 subunit alpha. In some embodiments, the first cytokine fragment (Cy1) comprises an amino acid sequence having at least 70%, 75%, 80%, 85%, 90%, 91%, 92%, 93%, 94%, 95%, 96%, 97%, 98%, 99%, or 100% sequence identity to a sequence of SEQ ID NO. 5. In some embodiments, the second cytokine fragment (Cy2) comprises an amino acid sequence having at least 70%, 75%, 80%, 85%, 90%, 91%, 92%, 93%, 94%, 95%, 96%, 97%, 98%, 99%, or 100% sequence identity to a sequence of SEQ ID NO. 6. In some embodiments, the cytokine comprises a linker positioned between the first cytokine fragment (Cy1) and the second cytokine fragment (Cy2). In some embodiments, the cytokine is an IL-12 variant comprising an amino acid sequence having at least 90%, 91%, 92%, 93%, 94%, 95%, 96%, 97%, 98%, 99%, or 100% sequence identity to SEQ ID NO. 7.

In some embodiments, the XTEN sequence consists of multiple non-overlapping sequence motifs, wherein the sequence motifs are selected from the sequence motifs of Tables 2a-2b. In some embodiments, the XTEN has from 40 to 3000 amino acids, or from 100 to 3000 amino acids. In some embodiments, the XTEN has at least 90%, at least 91%, at least 92%, at least 93%, at least 94%, at least 95%, at least 96%, at least 97%, at least 98%, or at least 99%, or 100% sequence identity to a sequence set forth in Tables 2a-2b.

In some embodiments, a binding activity of the cytokine, when linked to the XTEN in the fusion protein, to a corresponding cytokine receptor can be characterized by a half maximal effective concentration (EC50) at least 1.2 fold greater, at least 1.4 fold greater, at least 1.6 fold greater, at least 1.8 fold greater, at least 2.0 fold greater, at least 3.0 fold greater, at least 4.0 fold greater, at least 5.0 fold greater, at least 6.0 fold greater, at least 7.0 fold greater, at least 8.0 fold greater, at least 9.0 fold greater, or at least 10.0 fold greater than an EC50 characterizing a corresponding binding activity of the cytokine, when not linked to the XTEN, as determined in an in vitro binding assay. In some embodiments, the cytokine can be interleukin 12 (IL-12) and the corresponding cytokine receptor can be an interleukin 12 receptor (IL-12R). In some embodiments, the in vitro binding assay can utilize a genetically engineered reporter gene cell line configured to respond to binding of the cytokine to the corresponding cytokine receptor with a proportional expression of a reporter protein. In some embodiments, the in vitro binding assay can be a reporter gene activity assay.

In another aspect, the present disclosure provides a composition, comprising the fusion protein disclosed herein and at least one pharmaceutically acceptable carrier. In yet another aspect, the present disclosure provides uses of the subject composition in the preparation of a medicament for treating a disease in a subject in need thereof.

In a related aspect, the present disclosure provides a method of treating or preventing a disease or condition in a subject, the method comprising administering to a subject a therapeutically effective amount of a fusion protein or a composition comprising the fusion protein, all of which are disclosed herein. In some embodiments, the disease or condition can be a cancer, or a cancer-related disease or condition, or an inflammatory or autoimmune disease. In some embodiments, the disease or condition can be a cancer, or a cancer-related disease or condition. The diseases or conditions that can be treated with the subject fusion and composition include without limitation cancer, rheumatoid arthritis, multiple sclerosis, myasthenia gravis, systemic lupus erythematosus, Alzheimer's disease, Schizophrenia, viral infections, allergic asthma, retinal neurodegenerative processes, metabolic disorder, insulin resistance, and diabetic cardiomyopathy. In some embodiments, the disease or condition can be a cancer or a cancer-related disease or condition. Where desired, the subject fusion and composition can be used in conjunction with a therapeutically effective amount of at least one immune checkpoint inhibitor. Where desired, the mode of administration can be delivered intravenously, subcutaneously, or orally.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features and advantages of the invention may be further explained by reference to the following detailed description and accompanying drawings that sets forth illustrative embodiments.

FIG. 1A-FIG. 1G show schematic representations of exemplary BPXTEN fusion proteins (FIGS. 1A-G), all depicted in an N- to C-terminus orientation. FIG. 1A shows two different configurations of BPXTEN fusion proteins (100), each comprising a single biologically active protein (BP) and an XTEN, the first of which has an XTEN molecule (102) attached to the C-terminus of a BP (103), and the second of which has an XTEN molecule attached to the N-terminus of a BP (103). FIG. 1B shows two different configurations of BPXTEN fusion proteins (100), each comprising a single BP, a spacer sequence and an XTEN, the first of which has an XTEN molecule (102) attached to the C-terminus of a spacer sequence (104) and the spacer sequence attached to the C-terminus of a BP (103) and the second of which has an XTEN molecule attached to the N-terminus of a spacer sequence (104) and the spacer sequence attached to the N-terminus of a BP (103). FIG. 1C shows two different configurations of BPXTEN fusion proteins (101), each comprising two molecules of a single BP and one molecule of an XTEN, the first of which has an XTEN linked to the C-terminus of a first BP and that BP is linked to the C-terminus of a second BP, and the second of which is in the opposite orientation in which the XTEN is linked to the N-terminus of a first BP and that BP is linked to the N-terminus of a second BP. FIG. 1D shows two different configurations of BPXTEN fusion proteins (101), each comprising two molecules of a single BP, a spacer sequence and one molecule of an XTEN, the first of which has an XTEN linked to the C-terminus of a spacer sequence and the spacer sequence linked to the C-terminus of a first BP which is linked to the C-terminus of a second BP, and the second of which is in the opposite orientation in which the XTEN is linked to the N-terminus of a spacer sequence and the spacer sequence is linked to the N-terminus of a first BP that that BP is linked to the N-terminus of a second BP. FIG. 1E shows two different configurations of BPXTEN fusion proteins (101), each comprising two molecules of a single BP, a spacer sequence and one molecule of an XTEN, the first of which has an XTEN linked to the C-terminus of a first BP and the first BP linked to the C-terminus of a spacer sequence which is linked to the C-terminus of a second BP molecule, and the second of which is in the opposite configuration of XTEN linked to the N-terminus of a first BP which is linked to the N-terminus of a spacer sequence which in turn is linked to the N-terminus of a second molecule of BP. FIG. 1F shows two different configurations of BPXTEN fusion proteins (105), each comprising two molecules of a single BP, and two molecules of an XTEN, the first of which has a first XTEN linked to the C-terminus of a first BP which is linked to the C-terminus of a second XTEN that is linked to the C-terminus of a second molecule of BP, and the second of which is in the opposite configuration of XTEN linked to the N-terminus of a first BP linked to the N-terminus of a second XTEN linked to the N-terminus of a second BP. FIG. 1G shows a configuration (106) of a single BP linked to two XTEN at the N- and C-termini of the BP.

FIG. 2A-FIG. 2G is a schematic illustration of exemplary polynucleotide constructs of BPXTEN genes that encode the corresponding BPXTEN polypeptides of FIG. 1A-FIG. 1G; all depicted in a 5′ to 3′ orientation. In these illustrative examples the genes encode BPXTEN fusion proteins with one BP and XTEN (100); or two BP, one spacer sequence and one XTEN (201); two BP and two XTEN (205); or one BP and two XTEN (206). In these depictions, the polynucleotides encode the following components: XTEN (202), BP (203), and spacer amino acids that can include a cleavage sequence (204), with all sequences linked in frame.

FIG. 3A-FIG. 3E is a schematic illustration of an exemplary monomeric BPXTEN acted upon by an endogenously available protease and the ability of the monomeric fusion protein or the reaction products to bind to a target receptor on a cell surface, with subsequent cell signaling. FIG. 3A shows a BPXTEN fusion protein (101) in which a BP (103) and an XTEN (102) are linked by spacer sequences that contain a cleavable sequence (104), the latter being susceptible to MMP-13 protease (105). FIG. 3B shows the reaction products of a free BP, spacer sequence and XTEN. FIG. 3C shows the interaction of the reaction product free BP (103) or BPXTEN fusion protein (101) with target receptors (106) to BP on a cell surface (107). In this case, desired binding to the receptor is exhibited when BP has a free C-terminus, as evidenced by the binding of free BP (103) to the receptor while uncleaved fusion protein does not bind tightly to the receptor. FIG. 3D shows that the free BP (103), with high binding affinity, remains bound to the receptor (106), while an intact BPXTEN (101) is released from the receptor. FIG. 3E shows the bound BP has been internalized into an endosome (108) within the cell (107), illustrating receptor-mediated clearance of the bound BP and triggering cell signaling (109), portrayed as stippled cytoplasm.

FIG. 4 is a schematic flowchart of representative steps in the assembly, production and the evaluation of a XTEN.

FIG. 5 is a schematic flowchart of representative steps in the assembly of a BP-XTEN polynucleotide construct encoding a fusion protein. Individual oligonucleotides 501 are annealed into sequence motifs 502 such as a 12 amino acid motif (“12-mer”), which is subsequently ligated with an oligo containing BbsI, and KpnI restriction sites 503. Additional sequence motifs from a library are annealed to the 12-mer until the desired length of the XTEN gene 504 is achieved. The XTEN gene is cloned into a stuffer vector. The vector encodes a Flag sequence 506 followed by a stopper sequence that is flanked by BsaI, BbsI, and KpnI sites 507 and a cytokine gene 508, resulting in the gene 500 encoding a BP-XTEN fusion for incorporation into a BPXTEN combination.

FIG. 6 is a schematic flowchart of representative steps in the assembly of a gene encoding fusion protein comprising a biologically active protein (BP) and XTEN, its expression and recovery as a fusion protein, and its evaluation as a candidate BPXTEN product.

FIG. 7 illustrates the structural configuration of an exemplified XTENylated cytokine (i.e. a “XTENylated IL12” construct), having an amino acid sequence of SEQ ID NO: 2 (see Table B). The exemplified “XTENylated IL12” construct comprises a cleavage sequence capable of being cleaved by a mammalian protease. Upon the protease cleavage of the exemplified “XTENylated IL12” construct, a corresponding “de-XTENylated IL12” fragment and an “XTEN fragment” are released. Also illustrated is a reference cytokine construct (i.e. a “Reference IL12” construct), having an amino acid sequence of SEQ ID NO: 4 (see Table B), which contains the same IL12 moiety.

FIG. 8 illustrates reduced cytokine activity due to XTENylation. For example, an XTENylated (masked) interleukin-12 (IL12) composition (SEQ ID NO: 2) is at least 2-fold less active in inducing signal transducer and activator of transcription 4 (STAT-4) in 293 HEK IL-12 reporter cells relative to the corresponding protease-activated, de-XTENylated (unmasked) IL-12 composition. The protease treatment to de-XTENylate an XTENylated cytokine composition is illustrated in FIG. 7. The EC50 of the XTENylated IL12 (having a value of 167.0) is greater than the EC50 of the corresponding de-XTENylated IL12 (having a value of 79.4), indicating the masking ability of XTEN on IL12 proteins and, more generally, on cytokines.

FIG. 9A-FIG. 9B illustrate XTENylation-mediated reduction in cytokine binding. For example, FIG. 9A illustrates binding of an “XTENylated IL12” composition (SEQ ID NO: 2) and a “Reference IL12” composition without XTENylation (SEQ ID NO: 4) to 293 HEK-IL-12 reporter cells (HEK-Blue™ IL-12 cells (Invivogen, San Diego, Calif.)). The EC50 of the “XTENylated IL12” (having a value of ˜11.8) is greater than the EC50 of the “Reference IL12” (having a value of ˜4.5), indicating the ability (i.e. the masking effect) of an XTEN in interfering with the binding between the IL12 and the corresponding IL12 receptor. FIG. 9B illustrates the lack of binding of the “XTENylated IL12” and the “Reference IL12” compositions with IL12 receptor negative 293 HEK cells (control). As a further control, no binding was observed for the corresponding XTEN fragment (see FIG. 7) with either the IL12 reporter cells or the IL12-negative control cells.

FIG. 10A-10C. IL12-XPAC-4X structure and activity assays. FIG. 10A shows schematic structure of an exemplary IL12-XPAC-4X in which there are 4 XTEN chains on the IL-12 subunits. FIG. 10B shows schematic of IL12-XPAC-4X shown in FIG. 10A in which a transglutaminase tag (TG) tag is added. The TG tag is shown by the arrow. FIG. 10C HEK Blue activity assay for the PAC and XPACs of the two constructs from FIG. 10A and FIG. 10B.

FIG. 11A-11C. All XTENs mask activity. FIG. 11A shows activity with an exemplary construct that contains four XTEN moieties (AP2446). FIG. 11B shows activity with an exemplary construct that contains three XTEN moieties (AP2447). FIG. 11C shows activity with an exemplary construct that contains one XTEN moiety (AP2450).

FIG. 12A-12C. Design of three exemplary IL12-XPAC-4X constructs. FIG. 12A design of AC2582/AC2585, FIG. 12B design of AC3244/AC3247. FIG. 12C design of AC3245/AC3246.

FIG. 13 shows schematic of an exemplary XPAC further comprising a tumor binding domain.

FIG. 14 shows tumor regression results from an in vivo efficacy study performed in C57/Blk6 mice bearing MC38 tumors. Once established the tumors were treated with either diluent, rIL-12 at three different concentrations or IL-12-XPAC at two different concentrations. The data shown support the efficacy of IL-12 XPACs in producing tumor regression.

FIG. 15A shows the toxicity/body weight data obtained from the tumor-bearing mouse study shown in FIG. 14. FIG. 15B shows the effects of rIL12 and IL12 XPAC on the body weight of non-tumor bearing mice. These data demonstrate XPAC safety.

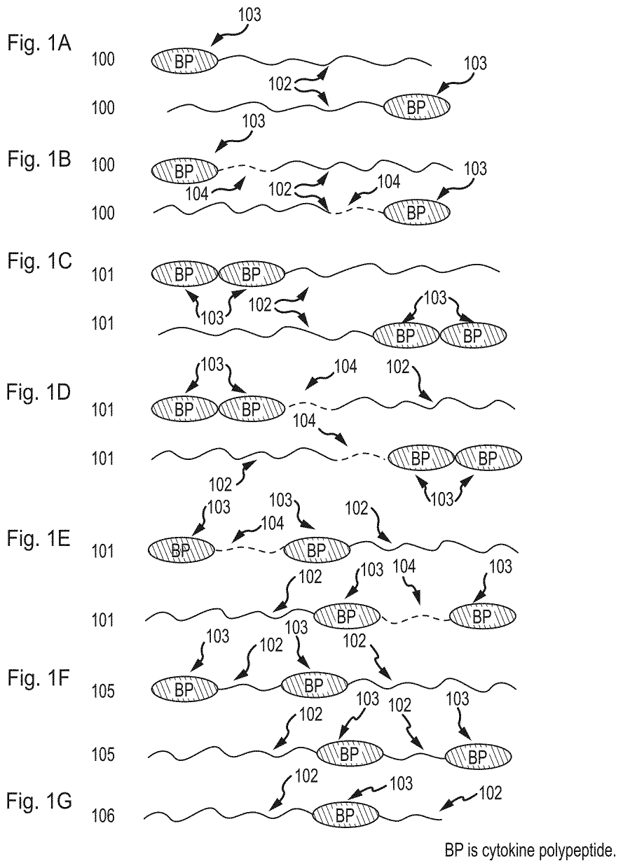

### DETAILED DESCRIPTION

While cytokines have potential to be potent therapeutics, even at low concentrations, these agents produce side effects that limit their practical application in a clinical setting. The present disclosure harnesses the therapeutic potential of cytokine-related compositions and related methods while controlling the deleterious effects of those powerful compounds. More specifically, the present disclosure relates to specific BPXTEN molecules known as Xtenylated Protease Activated Cytokines (XPACs) that are conditionally activated in the presence of proteases present in the tumor microenvironment. The present application is directed to methods and compositions for the preparation of XPACs. While the present disclosure presents certain examples with IL12, it should be understood that this disclosure is broadly applicable to any cytokine whose activity should preferably be attenuated until such a time that it is presented at the site of action. XPACs provide an effective method for overcoming tumor-induced immune suppression that can result from the role of IL12 in T- and NK-cell-mediated inflammatory responses.

As noted above, cytokines are potent immune agonists, however, the relatively narrow therapeutic window of this powerful class of compounds has limited their promise in a therapeutic setting. They have a short half-life, are extremely potent, and produce significant undesirable systemic effects and toxicities. In addition, the therapeutic window was further narrowed by the need to administer large quantities of cytokine in order to achieve the desired levels of cytokine at the intended site of cytokine action in the tumor or tumor microenvironment. As such, cytokines have until now failed to reach their potential in the clinical setting for the treatment of tumors.

The present invention overcomes the toxicity and short half-life shortcomings that have hampered the clinical use of cytokines in oncology. The XPACs of the present invention contain cytokine polypeptides that have receptor agonist activity. But in the context of the XPAC, the cytokine receptor agonist activity is attenuated and the circulating half-life is extended. The XPACs include protease cleave sites, which are cleaved by proteases that are associated with a desired site of cytokine activity (e.g., a tumor), and are typically enriched or selectively present at the site of desired activity. Thus, the XPACs are preferentially (or selectively) and efficiently cleaved at the desired site of action. This limits the cytokine activity substantially to the desired site of activity, such as the tumor microenvironment. Protease cleavage at the desired site of activity, such as in a tumor microenvironment, releases a form of the cytokine from the XPAC that is much more active as a cytokine receptor agonist than the XPAC which has the XTEN molecule attached. The form of the cytokine that is released upon cleavage of XTEN from the XPAC typically has a short half-life, which is often substantially similar to the half-life of the naturally occurring cytokine. This advantageously limits the cytokine activity to the tumor microenvironment. Even though the half-life of the XPAC is extended, toxicity is dramatically reduced or eliminated because the circulating XPAC is attenuated and active cytokine is targeted to the tumor microenvironment. The XPACs described herein, for the first time, enable the administration of an effective therapeutic dose of a cytokine to treat tumors with the activity of the cytokine substantially limited to the tumor microenvironment, and dramatically reduces or eliminates unwanted systemic effects and toxicity of the cytokine.

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

As used herein, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

The term “cytokine” is well-known to those of skill in the art and refers to any of a class of immunoregulatory proteins that are secreted by cells especially of the immune system and are immunomodulators. Cytokine polypeptides that can be used in the XPACs disclosed herein include, but are not limited to interleukins, such as IL-1, IL-1.alpha., IL-2, IL-3, IL-4, IL-5, IL-6, IL-7, IL-8, IL-9, IL-10, IL-11, IL-12, IL-13, IL-14, IL-15, IL-16, IL-17, IL-18, IL-21 and IL-25, transforming growth factors, such as TGF-.alpha. and TGF-.beta. (e.g., TGFbeta1, TGFbeta2, TGFbeta3); interferons, such as interferon-.alpha., interferon-.beta., interferon-.gamma., interferon-kappa and interferon-omega; tumor necrosis factors, such as tumor necrosis factor alpha and lymphotoxin; chemokines (e.g., C-X-C motif chemokine 10 (CXCL10), CCL19, CCL20, CCL21), and granulocyte macrophage-colony stimulating factor (GM-CS), as well as functional fragments thereof that retain receptor agonist activity. “Chemokine” is a term of art that refers to any of a family of small cytokines with the ability to induce directed chemotaxis in nearby responsive cells.

As used herein, the terms “activatable,” “activate,” “induce,” and “inducible” refer to the ability of a protein, i.e. a cytokine, that is part of a XPAC, to bind its receptor and effectuate activity upon cleavage of the XTEN from the XPAC.

Those of skill in the art understand the term “half-life extension” is used to mean that as compared to a cytokine that is part of the XPAC, the XPAC that increases the serum half-life and improves pK, for example, by altering its size (e.g., to be above the kidney filtration cutoff), shape, hydrodynamic radius, charge, or parameters of absorption, biodistribution, metabolism, and elimination.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide may share no more than 99%, 98%, 95%, 90%, 80%, 70%, 60%, 50% or even less amino acid sequence identity as compared to a natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

A “fragment” is a truncated form of a native biologically active protein that retains at least a portion of the therapeutic and/or biological activity. A “variant” is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity with the reference biologically active protein. As used herein, the term “biologically active protein moiety” includes proteins modified deliberately, as for example, by site directed mutagenesis, insertions, or accidentally through mutations.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two more chemical elements or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature). The terms “link,” “linked,” and “linking” are used in the broadest sense, and are specifically intended to include both covalent and non-covalent attachment of a moiety of the therapeutic agent to another moiety of the therapeutic agent in a direct or indirect way. The term “linked directly,” as used herein in the context of a therapeutic agent, generally refers to a structure in which a moiety is connected with or attached to another moiety without an intervening tether. The term “linked indirectly,” as used herein in the context of a therapeutic agent, generally refers to a structure in which a moiety of the therapeutic agent is connected with, or attached to, another moiety of the therapeutic agent via an intervening tether.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples ofpolynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60 to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point © for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2 and chapter 9. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, for example, as defined by a particular SEQ ID number, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “non-repetitiveness” as used herein in the context of a polypeptide refers to a lack or limited degree of internal homology in a peptide or polypeptide sequence. The term “substantially non-repetitive” can mean, for example, that there are few or no instances of four contiguous amino acids in the sequence that are identical amino acid types or that the polypeptide has a subsequence score (defined infra) of 10 or less or that there isn't a pattern in the order, from N- to C-terminus, of the sequence motifs that constitute the polypeptide sequence. The term “repetitiveness” as used herein in the context of a polypeptide refers to the degree of internal homology in a peptide or polypeptide sequence. In contrast, a “repetitive” sequence may contain multiple identical copies of short amino acid sequences. For instance, a polypeptide sequence of interest may be divided into n-mer sequences and the number of identical sequences can be counted. Highly repetitive sequences contain a large fraction of identical sequences while non-repetitive sequences contain few identical sequences. In the context of a polypeptide, a sequence can contain multiple copies of shorter sequences of defined or variable length, or motifs, in which the motifs themselves have non-repetitive sequences, rendering the full-length polypeptide substantially non-repetitive. The length of polypeptide within which the non-repetitiveness is measured can vary from 3 amino acids to about 200 amino acids, about from 6 to about 50 amino acids, or from about 9 to about 14 amino acids. “Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The term “t_{1/2}” as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t_{1/2}”, “terminal half-life”, “elimination half-life” and “circulating half-life” are used interchangeably herein.

“Apparent Molecular Weight Factor” or “Apparent Molecular Weight” are related terms referring to a measure of the relative increase or decrease in apparent molecular weight exhibited by a particular amino acid sequence. The Apparent Molecular Weight is determined using size exclusion chromatography (SEC) and similar methods compared to globular protein standards and is measured in “apparent kD” units. The Apparent Molecular Weight Factor is the ratio between the Apparent Molecular Weight and the actual molecular weight; the latter predicted by adding, based on amino acid composition, the calculated molecular weight of each type of amino acid in the composition.

The “hydrodynamic radius” or “Stokes radius” is the effective radius (Rₕ in nm) of a molecule in a solution measured by assuming that it is a body moving through the solution and resisted by the solution's viscosity. In the embodiments of the invention, the hydrodynamic radius measurements of the XTEN fusion proteins correlate with the ‘Apparent Molecular Weight Factor’, which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refer to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 25° C. to about 38° C., and preferably from about 35° C. to about 37° C.

A “reactive group” is a chemical structure that can be coupled to a second reactive group. Examples for reactive groups are amino groups, carboxyl groups, sulfhydryl groups, hydroxyl groups, aldehyde groups, azide groups. Some reactive groups can be activated to facilitate coupling with a second reactive group. Examples for activation are the reaction of a carboxyl group with carbodiimide, the conversion of a carboxyl group into an activated ester, or the conversion of a carboxyl group into an azide function.

“Controlled release agent”, “slow release agent”, “depot formulation” or “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The terms “antigen”, “target antigen” or “immunogen” are used interchangeably herein to refer to the structure or binding determinant that an antibody fragment or an antibody fragment-based therapeutic binds to or has specificity against.

The term “payload” as used herein refers to a protein or peptide sequence that has biological or therapeutic activity; the counterpart to the pharmacophore of small molecules. Examples of payloads include, but are not limited to, cytokines, enzymes, hormones and blood and growth factors. Payloads can further comprise genetically fused or chemically conjugated moieties such as chemotherapeutic agents, antiviral compounds, toxins, or contrast agents. These conjugated moieties can be joined to the rest of the polypeptide via a linker which may be cleavable or non-cleavable.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” or “palliating” or “ameliorating” is used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Thus, for example, treatment refers to a method of reducing the effects of a disease or condition or symptom of the disease or condition. Thus, in the disclosed method, treatment can refer to at least about 10%, at least about 20%, at least about 30%, at least about 40%, at least about 50%, at least about 60%, at least about 70%, at least about 80%, at least about 90%, or substantially complete reduction in the severity of an established disease or condition or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. Also, a therapeutic benefit is achieved with the eradication or amelioration of one or more of the physiological symptoms associated with the underlying disease condition such that an improvement is observed in the subject, notwithstanding that the subject may still be afflicted with the underlying disorder. For prophylactic benefit, the compositions may be administered to a subject at risk of developing a particular disease or condition, or to a subject reporting one or more of the physiological symptoms of a disease, even though a diagnosis of this disease may not have been made. It is understood that treatment does not necessarily refer to a cure or complete ablation of the disease, condition, or symptoms of the disease or condition.

A “therapeutic effect”, as used herein, refers to a physiologic effect, including but not limited to the cure, mitigation, amelioration, or prevention of disease or condition in humans or other animals, or to otherwise enhance physical or mental wellbeing of humans or animals, caused by a fusion polypeptide of the invention other than the ability to induce the production of an antibody against an antigenic epitope possessed by the biologically active protein. Determination of a therapeutically effective amount is well within the capability of those skilled in the art, especially in light of the detailed disclosure provided herein.

The terms “therapeutically effective amount” and “therapeutically effective dose”, as used herein, refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. The disease or condition can refer to a disorder or a disease.

The term “therapeutically effective dose regimen”, as used herein, refers to a schedule for consecutively administered doses of a biologically active protein, either alone or as a part of a fusion protein composition, wherein the doses are given in therapeutically effective amounts to result in sustained beneficial effect on any symptom, aspect, measured parameter or characteristics of a disease state or condition.

As used herein, the terms “prevent”, “preventing”, and “prevention” of a disease or disorder refers to an action, for example, administration of the chimeric polypeptide or nucleic acid sequence encoding the chimeric polypeptide, that occurs before or at about the same time a subject begins to show one or more symptoms of the disease or disorder, which inhibits or delays onset or exacerbation of one or more symptoms of the disease or disorder.

As used herein, references to “decreasing”, “reducing”, or “inhibiting” include a change of at least about 10%, of at least about 20%, of at least about 30%, of at least about 40%, of at least about 50%, of at least about 60%, of at least about 70%, of at least about 80%, of at least about 90% or greater as compared to a suitable control level. Such terms can include but do not necessarily include complete elimination of a function or property, such as agonist activity.

An “attenuated cytokine receptor agonist” is a cytokine receptor agonist that has decreased receptor agonist activity as compared to the cytokine receptor's naturally occurring agonist. An attenuated cytokine agonist may have at least about 10 times, at least about 50 times, at least about 100 times, at least about 250 times, at least about 500 times, at least about 1000 times or less agonist activity as compared to the receptor's naturally occurring agonist. When a XPAC that contains a cytokine polypeptide as described herein is described as “attenuated” or having “attenuated activity”, it is meant that the XPAC is an attenuated cytokine receptor agonist.

### General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4^{th} edition, John Wiley & Sons, Somerset, N J, 2000, the contents of which are incorporated in their entirety herein by reference.

### Cytokines for Use in XPACs

In general, the therapeutic use of cytokines is strongly limited by their systemic toxicity. TNF, for example, was originally discovered for its capacity of inducing the hemorrhagic necrosis of some tumors, and for its in vitro cytotoxic effect on different tumoral lines, but it subsequently proved to have strong pro-inflammatory activity, which can, in case of overproduction conditions, dangerously affect the human body. As the systemic toxicity is a fundamental problem with the use of pharmacologically active amounts of cytokines in humans, novel derivatives and therapeutic strategies are now under evaluation, aimed at reducing the toxic effects of this class of biological effectors while keeping their therapeutic efficacy.

A preferred cytokine for use in production of XPACs is Interleukin-12 (IL-12). IL-12 is a disulfide-linked heterodimer of two separately encoded subunits (p35 and p40), which are linked covalently to give rise to the so-called bioactive heterodimeric (p70) molecule. Apart from forming heterodimers (IL-12 and IL-23), the p40 subunit is also secreted as a monomer (p40) and a homodimer (p40₂). It is known in the art that synthesis of the heterodimer as a single chain with a linker connecting the p35 to the p40 subunit preserves the full biological activity of the heterodimer. IL-12 plays a critical role in the early inflammatory response to infection and in the generation of Th1 cells, which favor cell-mediated immunity. It has been found that overproduction of IL-12 can be dangerous to the host because it is involved in the pathogenesis of a number of autoimmune inflammatory diseases (e.g. MS, arthritis, type 1 diabetes).

The IL-12 receptor (IL-12R) is a heterodimeric complex consisting of IL-12Rβ1 and IL-12Rβ2 chains expressed on the surface of activated T-cells and natural killer cells. The IL-12Rβ1 chain binds to the IL-12p40 subunit, whereas IL-12p35 in association with IL-12Rβ2 confers an intracellular signaling ability. Signal transduction through IL-12R induces phosphorylation of Janus kinase (Jak2) and tyrosine kinase (Tyk2), that phosphorylate and activate signal transducer and activator of transcription (STAT)1, STAT3, STAT4, and STATS. The specific cellular effects of IL-12 are due mainly to activation of STAT4. IL-12 induces natural killer and T-cells to produce cytokines, in particular interferon (IFN)γ, that mediate many of the proinflammatory activities of IL-12, including CD4+ T-cell differentiation toward the Th1 phenotype.

IL-2 exerts both stimulatory and regulatory functions in the immune system and is, along with other members of the common γ-chain cytokine family, central to immune homeostasis. IL-2 mediates its action by binding to IL-2 receptors (IL-2R), consisting of either trimeric receptors made of IL-2Rα(CD25), IL-2Rβ (CD122), and IL-2R-γ (γ-c, CD132) chains or dimeric β γ IL-2Rs. Both IL-2R variants are able to transmit signal upon IL-2 binding. However, trimeric αβγ IL-2Rs have a roughly 10-100 times higher affinity for IL-2 than dimeric βγ IL-2Rs (3), implicating that CD25 confers high-affinity binding of IL-2 to its receptor but is not crucial for signal transduction. Trimeric IL-2Rs are found on activated T cells and CD4+ forkhead box P3 (FoxP3)+T regulatory cells (Treg), which are sensitive to IL-2 in vitro and in vivo. Conversely, antigen-experienced (memory) CD8+, CD44 high memory-phenotype (MP) CD8+, and natural killer (NK) cells are endowed with high levels of dimeric βγ IL-2Rs, and these cells also respond vigorously to IL-2 in vitro and in vivo.

Expression of the high-affinity IL-2R is critical for endowing T cells to respond to low concentrations of IL-2 that is transiently available in vivo. IL-2Ra expression is absent on naive and memory T cells but is induced after antigen activation. IL-2RP is constitutively expressed by NK, NKT, and memory CD8+ T cells but is also induced on naive T cells after antigen activation. γ-chain is much less stringently regulated and is constitutively expressed by all lymphoid cells. Once the high-affinity IL-2R is induced by antigen, IL-2R signaling upregulates the expression of IL-2Ra in part through Stat5-dependent regulation of Il2ra transcription. This process represents a mechanism to maintain expression of the high-affinity IL-2R and sustain IL-2 signaling while there remains a source of IL-2.

Interleukin-15 (IL-15), another member of the 4-alpha-helix bundle family of cytokines, has also emerged as an immunomodulator for the treatment of cancer. IL-15 is initially captured via IL-15Ra, which is expressed on antigen-presenting dendritic cells, monocytes and macrophages. IL-15 exhibits broad activity and induces the differentiation and proliferation of T, B and natural killer (NK) cells via signaling through the IL-15/IL-2-R-β (CD122) and the common γ chain (CD132). It also enhances cytolytic activity of CD8+ T cells and induces long-lasting antigen-experienced CD8+CD44 memory T cells. IL-15 stimulates differentiation and immunoglobulin synthesis by B cells and induces maturation of dendritic cells. It does not stimulate immunosuppressive T regulatory cells (Tregs). Thus, boosting IL-15 activity selectively in the tumor microenvironment could enhance innate and specific immunity and fight tumors.

Interleukin-7 (IL-7), also of the IL-2/IL-15 family, is a well-characterized pleiotropic cytokine, and is expressed by stromal cells, epithelial cells, endothelial cells, fibroblasts, smooth muscle cells and keratinocytes, and following activation, by dendritic cells (Alpdogan et al., 2005). Although it was originally described as a growth and differentiation factor for precursor B lymphocytes, subsequent studies have shown that IL-7 is critically involved in T-lymphocyte development and differentiation. Interleukin-7 signaling is essential for optimal CD8 T-cell function, homeostasis and establishment of memory (Schluns et al., 2000); it is required for the survival of most T-cell subsets, and its expression has been proposed to be important for regulating T-cell numbers.

IL-7 has a potential role in enhancing immune reconstitution in cancer patients following cytotoxic chemotherapy. IL-7 therapy enhances immune reconstitution and can augment even limited thymic function by facilitating peripheral expansion of even small numbers of recent thymic emigrants. Therefore, IL-7 therapy could potentially repair the immune system of patients who have been depleted by cytotoxic chemotherapy and may be an attractive candidate for XPAC production.

Regulatory T cells actively suppress activation of the immune system and prevent pathological self-reactivity and consequent autoimmune disease. Developing drugs and methods to selectively activate regulatory T cells for the treatment of autoimmune disease is the subject of intense research and, until the development of the present invention, which can selectively deliver active interleukins at the site of inflammation, has been largely unsuccessful. Regulatory T cells (Treg) are a class of CD4+CD25+ T cells that suppress the activity of other immune cells. Treg are central to immune system homeostasis, and play a major role in maintaining tolerance to self-antigens and in modulating the immune response to foreign antigens. Multiple autoimmune and inflammatory diseases, including Type 1 Diabetes (T1D), Systemic Lupus Erythematosus (SLE), and Graft-versus-Host Disease (GVHD) have been shown to have a deficiency of Treg cell numbers or Treg function.

As such, there is great interest in the development of therapies that boost the numbers and/or function of Treg cells. One approach is treatment with low dose Interleukin-2 (IL-2). Treg cells characteristically express high constitutive levels of the high affinity IL-2 receptor, IL2Rαβγ which is composed of the subunits IL2Rα (CD25), IL2Rβ (CD122), and IL2Rγ (CD132), and Treg cell growth has been shown to be dependent on IL-2. Conversely, immune activation has also been achieved using IL-2, and recombinant IL-2 (Proleukin®) has been approved to treat certain cancers. High-dose IL-2 is used for the treatment of patients with metastatic melanoma and metastatic renal cell carcinoma with a long-term impact on overall survival.

Clinical trials of low-dose IL-2 treatment of chronic GVHD and HCV-associated autoimmune vasculitis patients demonstrated increased Treg levels and signs of clinical efficacy. The rationale for using so-called low dose IL-2 was to exploit the high IL-2 affinity of the trimeric IL-2 receptor which is constitutively expressed on Tregs while leaving other T cells which do not express the high affinity receptor in the inactivated state. Proleukin® (Prometheus Laboratories, San Diego, Calif.), the recombinant form of IL-2 used in these trials, is associated with high toxicity. Aldesleukin, at high doses, is approved for the treatment of metastatic melanoma and metastatic renal cancer, but its side effects are so severe that its use is only recommended in a hospital setting with access to intensive care.

The clinical trials of IL-2 in autoimmune diseases have employed lower doses of IL-2 in order to target Treg cells, because Treg cells respond to lower concentrations of IL-2 than many other immune cell types due to their expression of IL2R alpha. However, even these lower doses resulted in safety and tolerability issues, and the treatments used have employed daily subcutaneous injections, either chronically or in intermittent 5-day treatment courses. Therefore, there is a need for an autoimmune disease therapy that potentiates Treg cell numbers and function, that targets Treg cells more specifically than IL-2, that is safer and more tolerable, and that is administered less frequently. This low therapeutic window for IL-2 is played out across other cytokine therapies.

One approach for improving the therapeutic index of cytokine-based therapy for autoimmune diseases was to use variants of IL-2 that are selective for Treg cells relative to other immune cells. IL-2 receptors are expressed on a variety of different immune cell types, including T cells, NK cells, eosinophils, and monocytes, and this broad expression pattern likely contributes to its pleiotropic effect on the immune system and high systemic toxicity. In particular, activated T effector cells express IL2Rββγ, as do pulmonary epithelial cells. But, activating T effector cells runs directly counter to the goal of down-modulating and controlling an immune response, and activating pulmonary epithelial cells leads to known dose-limiting side effects of IL-2 including pulmonary edema. In fact, the major side effect of high-dose IL-2 immunotherapy is vascular leak syndrome (VLS), which leads to accumulation of intravascular fluid in organs such as lungs and liver with subsequent pulmonary edema and liver cell damage. There is no treatment of VLS other than withdrawal of IL-2. Low-dose IL-2 regimens have been tested in patients to avoid VLS, however, at the expense of suboptimal therapeutic results.

Treatment with interleukin cytokines other than IL-2 has been even more limited. IL-15 displays immune cell stimulatory activity similar to that of IL-2 but without the same inhibitory effects, thus making it a promising immunotherapeutic candidate. Clinical trials of recombinant human IL-15 for the treatment of metastatic malignant melanoma or renal cell cancer demonstrated appreciable changes in immune cell distribution, proliferation, and activation and suggested potential antitumor activity. IL-15 therapy is known to be associated with undesired and toxic effects, such as exacerbating certain leukemias, graft-versus-host disease, hypotension, thrombocytopenia, and liver injury.

IL-7 promotes lymphocyte development in the thymus and maintains survival of naive and memory T cell homeostasis in the periphery. Moreover, it is important for the organogenesis of lymph nodes (LN) and for the maintenance of activated T cells recruited into the secondary lymphoid organs (SLOs). In clinical trials of IL-7, patients receiving IL-7 showed increases in both CD4+ and CD8+ T cells, with no significant increase in regulatory T cell numbers as monitored by FoxP3 expression. In clinical trials reported in 2006, 2008 and 2010, patients with different kinds of cancers such as metastatic melanoma or sarcoma were injected subcutaneously with different doses of IL-7. Little toxicity was seen except for transient fevers and mild erythema. Circulating levels of both CD4+ and CD8+ T cells increased significantly and the number of Treg reduced. TCR repertoire diversity increased after IL-7 therapy. However, the anti-tumor activity of IL-7 was not well evaluated. Results suggest that IL-7 therapy could enhance and broaden immune responses.

IL-12 is a pleiotropic cytokine, that creates an interconnection between the innate and adaptive immunity. IL-12 was first described as a factor secreted from PMA-induced EBV-transformed B-cell lines. Based on its actions, IL-12 has been designated as cytotoxic lymphocyte maturation factor and natural killer cell stimulatory factor. Due to bridging the innate and adaptive immunity and potently stimulating the production of IFNgamma., a cytokine coordinating natural mechanisms of anticancer defense, IL-12 seemed ideal candidate for tumor immunotherapy in humans. However, severe side effects associated with systemic administration of IL-12 in clinical investigations and the very narrow therapeutic index of this cytokine markedly hampered the use of this cytokine in cancer patients. Approaches to IL-12 therapy in which delivery of the cytokine is tumor-targeted, which may diminish some of the previous issues with IL-12 therapy, are currently in clinical trials for cancers.

The direct use of IL-2 as an agonist to bind the IL-2R and modulate immune responses therapeutically has been problematic due its well-documented therapeutic risks, e.g., its short serum half-life and high toxicity. These risks have also limited the therapeutic development and use of other cytokines. New forms of cytokines that reduce these risks are needed. Disclosed herein are compositions and methods comprising conditionally active IL-12 and other cytokines designed to address the risks associated with conventional cytokine therapy and provide much needed immunomodulatory therapeutics.

Cytokines, including interleukins (e.g., IL-2, IL-7, IL-12, IL-15, IL-18, IL-21 IL-23), interferons (IFNs, including IFNalpha, IFNbeta and IFNgamma), tumor necrosis factors (e.g., TNFalpha, lymphotoxin), transforming growth factors (e.g., TGFbeta1l, TGFbeta2, TGFbeta3), chemokines (C-X-C motif chemokine 10 (CXCL10), CCL19, CCL20, CCL21), and granulocyte macrophage-colony stimulating factor (GM-CS) are highly potent when administered to patients. Forming XPACs with these molecules could make them more readily amenable for use in a therapeutic setting.

As used herein, “chemokine” means a family of small cytokines with the ability to induce directed chemotaxis in nearby responsive cells Cytokines can provide powerful therapy, but are accompanied by undesired effects that are difficult to control clinically and which have limited the clinical use of cytokines. This disclosure relates to new forms of cytokines that can be used in patients with reduced or eliminated undesired effects. In particular, this disclosure relates to pharmaceutical compositions including chimeric polypeptides (XPACs), nucleic acids encoding XPACs and pharmaceutical formulations of the foregoing that contain cytokines or active fragments or muteins of cytokines that have decreased cytokine receptor activating activity in comparison to the corresponding cytokine. However, under selected conditions or in a selected biological environment the chimeric polypeptides activate their cognate receptors, often with the same or higher potency as the corresponding naturally occurring cytokine. As described herein, this is typically achieved using a cytokine blocking moiety that blocks or inhibits the receptor activating function of the cytokine, active fragment or mutein thereof under general conditions but not under selected conditions, such as those present at the desired site of cytokine activity (e.g., an inflammatory site or a tumor).

While the present application is exemplified using IL-12 as the exemplary cytokine, those of skill in the art will understand that the teachings provided herein may readily be adapted for and describe and enable the use of XPACs formed from other cytokines, fragments and muteins, such as IL-2, IL-7, IL-12, IL-15, IL-18, IL-21 IL-23, IFNalpha, IFNbeta, IFNgamma, TNFalpha, lymphotoxin, TGF-beta1, TGFbeta2, TGFbeta3, GM-CSF, CXCL10, CCL19, CCL20, CCL21 and functional fragments or muteins of any of the foregoing.

Various elements ensure the delivery and activity of the cytokine in the XPACs of the invention preferentially at the site of desired cytokine activity and to severely limit systemic exposure to the cytokine via XTENylation which allows serum half-life extension for the cytokine of interest. In this serum half-life extension strategy, the XPAC may circulates for extended times (preferentially 1-2 or more weeks) but the activated version from which the XTEN sequence has been cleaved has the typical serum half-life of the cytokine.

By comparison to an XPAC, the serum half-life of the underlying cytokine administered intravenously is only about 10 minutes due to distribution into the total body extracellular space. Subsequently, the cytokine is metabolized by the kidneys with a half-life of 2.5 hours.

In some embodiments of this invention, the XPAC comprises a release segment which is cleaved at the site of action (e.g., by inflammation-specific or tumor-specific proteases) thereby releasing the cytokine's full activity at the desired site and also separating it from the half-life extension of the uncleaved (XPAC) version. In such embodiments, the fully active and free cytokine would have very different pharmacokinetic (pK) properties--a half-life of hours instead of weeks. In addition, exposure to active cytokine is limited to the site of desired cytokine activity (e.g., an inflammatory site or the tumor microenvironment) and systemic exposure to active cytokine, and associated toxicity and side effects, are reduced.

Creating XPACs from cytokines is an elegant mechanism by which to improve the use of cytokines, as immunostimulatory agents, for example for treating cancer. For example, in this aspect, the pharmacokinetics and/or pharmacodynamics of the cytokine (e.g., IL-2, IL-7, IL-12, IL-15, IL-18, IL-21 IL-23, IFNalpha, IFNbeta and IFNgamma, TNFalpha, lymphotoxin, TGFbeta1, TGFbeta2, TGFbeta3 GM-CSF, CXCL10, CCL19, CCL20, and CCL21 can be tailored to maximally activate effector cells (e.g., effect T cells, NK cells) and/or cytotoxic immune response promoting cells (e.g., induce dendritic cell maturation) at a site of desired activity, such as in a tumor or tumor microenvironment, but preferably not systemically.

Thus, provided herein are pharmaceutical compositions comprising XPAcs that are comprised of at least one cytokine polypeptide, such as interleukins (e.g., IL-2, IL-7, IL-12, IL-15, IL-18, IL-21, IL-23), interferons (IFNs, including IFNalpha, IFNbeta and IFNgamma), tumor necrosis factors (e.g., TNFalpha, lymphotoxin), transforming growth factors (e.g., TGFbeta1, TGFbeta2, TGFbeta3), chemokines (e.g. CXCL10, CCL19, CCL20, CCL21) and granulocyte macrophage-colony stimulating factor (GM-CS) or a functional fragment or mutein of any of the foregoing.

Preferably, the cytokine polypeptides (including functional fragments) that are included in the XPACs disclosed herein are not mutated or engineered to alter the properties of the naturally occurring cytokine, including receptor binding affinity and specificity or serum half-life. However, changes in amino acid sequence from naturally occurring (including wild type) cytokine are acceptable to facilitate cloning and to achieve desired expression levels.

### Extended Recombinant Polypeptides

The present invention provides compositions comprising extended recombinant polypeptides (“XTEN” or “XTENs”). In some embodiments, XTEN are generally extended length polypeptides with non-naturally occurring, substantially non-repetitive sequences that are composed mainly of small hydrophilic amino acids, with the sequence having a low degree or no secondary or tertiary structure under physiologic conditions.

In one aspect of the invention, XTEN polypeptide compositions are disclosed that are useful as fusion partners that can be linked to biologically active proteins (“BP”), resulting in a BPXTEN fusion proteins (e.g., monomeric fusions). XTENs can have utility as fusion protein partners in that they can confer certain chemical and pharmaceutical properties when linked to a biologically active protein to a create a fusion protein. Such desirable properties include but are not limited to enhanced pharmacokinetic parameters and solubility characteristics, amongst other properties described below. Such fusion protein compositions may have utility to treat certain diseases, disorders or conditions, as described herein. As used herein, “XTEN” specifically excludes antibodies or antibody fragments such as single-chain antibodies, Fc fragments of a light chain or a heavy chain.

In some embodiments, XTEN are long polypeptides having greater than about 100 to about 3000 amino acid residues, preferably greater than 400 to about 3000 residues when used as a single sequence, and cumulatively have greater than about 400 to about 3000 amino acid residues when more than one XTEN unit is used in a single fusion protein or conjugate. In other cases, where an increase in half-life of the fusion protein is not needed but where an increase in solubility or other physico/chemical property for the biologically active protein fusion partner is desired, an XTEN sequence shorter than 100 amino acid residues, such as about 96, or about 84, or about 72, or about 60, or about 48, or about 36 amino acid residues may be incorporated into a fusion protein composition with the BP to effect the property.

The selection criteria for the XTEN to be linked to the biologically active proteins to create the inventive fusion proteins generally relate to attributes of physical/chemical properties and conformational structure of the XTEN that can be, in turn, used to confer enhanced pharmaceutical and pharmacokinetic properties to the fusion proteins. The XTEN of the present invention may exhibit one or more of the following advantageous properties: conformational flexibility, enhanced aqueous solubility, high degree of protease resistance, low immunogenicity, low binding to mammalian receptors, and increased hydrodynamic (or Stokes) radii; properties that can make them particularly useful as fusion protein partners. Non-limiting examples of the properties of the fusion proteins comprising BP that may be enhanced by XTEN include increases in the overall solubility and/or metabolic stability, reduced susceptibility to proteolysis, reduced immunogenicity, reduced rate of absorption when administered subcutaneously or intramuscularly, and enhanced pharmacokinetic properties such as terminal half-life and area under the curve (AUC), slower absorption after subcutaneous or intramuscular injection (compared to BP not linked to XTEN) such that the Cₘₐₓ is lower, which may, in turn, result in reductions in adverse effects of the BP that, collectively, can result in an increased period of time that a fusion protein of a BPXTEN composition administered to a subject remains within a therapeutic window, compared to the corresponding BP component not linked to XTEN.

A variety of methods and assays are known in the art for determining the physical/chemical properties of proteins such as the fusion protein compositions comprising the inventive XTEN; properties such as secondary or tertiary structure, solubility, protein aggregation, melting properties, contamination and water content. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Arnau et al, Prot Expr and Purif (2006) 48, 1-13. Application of these methods to the invention would be within the grasp of a person skilled in the art.

Typically, the XTEN component of the fusion proteins are designed to behave like denatured peptide sequences under physiological conditions, despite the extended length of the polymer. Denatured describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some cases, the invention provides XTEN sequences that, under physiologic conditions, can resemble denatured sequences largely devoid in secondary structure. In other cases, the XTEN sequences can be substantially devoid of secondary structure under physiologic conditions. “Largely devoid,” as used in this context, means that less than 50% of the XTEN amino acid residues of the XTEN sequence contribute to secondary structure as measured or determined by the means described herein. “Substantially devoid,” as used in this context, means that at least about 60%, or about 70%, or about 80%, or about 90%, or about 95%, or at least about 99% of the XTEN amino acid residues of the XTEN sequence do not contribute to secondary structure, as measured or determined by the means described herein.

A variety of methods have been established in the art to discern the presence or absence of secondary and tertiary structures in a given polypeptide. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra. Secondary structure can also be predicted for a polypeptide sequence via certain computer programs or algorithms, such as the well-known Chou-Fasman algorithm (Chou, P. Y., et al. (1974) Biochemistry, 13: 222-45) and the Gamier-Osguthorpe-Robson (“GOR”) algorithm (Gamier J, Gibrat J F, Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure).

In some cases, the XTEN sequences used in the inventive fusion protein compositions can have an alpha-helix percentage ranging from 0% to less than about 5% as determined by a Chou-Fasman algorithm. In other cases, the XTEN sequences of the fusion protein compositions can have a beta-sheet percentage ranging from 0% to less than about 5% as determined by a Chou-Fasman algorithm. In some cases, the XTEN sequences of the fusion protein compositions can have an alpha-helix percentage ranging from 0% to less than about 5% and a beta-sheet percentage ranging from 0% to less than about 5% as determined by a Chou-Fasman algorithm. In preferred embodiments, the XTEN sequences of the fusion protein compositions will have an alpha-helix percentage less than about 2% and a beta-sheet percentage less than about 2%. In other cases, the XTEN sequences of the fusion protein compositions can have a high degree of random coil percentage, as determined by a GOR algorithm. In some embodiments, an XTEN sequence can have at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, and most preferably at least about 99% random coil, as determined by a GOR algorithm.

### Non-Repetitive Sequences

XTEN sequences of the subject compositions can be substantially non-repetitive. In general, repetitive amino acid sequences have a tendency to aggregate or form higher order structures, as exemplified by natural repetitive sequences such as collagens and leucine zippers, or form contacts resulting in crystalline or pseudocrystaline structures. In contrast, the low tendency of non-repetitive sequences to aggregate enables the design of long-sequence XTENs with a relatively low frequency of charged amino acids that would be likely to aggregate if the sequences were otherwise repetitive. Typically, the BPXTEN fusion proteins comprise XTEN sequences of greater than about 100 to about 3000 amino acid residues, preferably greater than 400 to about 3000 residues, wherein the sequences are substantially non-repetitive. In one embodiment, the XTEN sequences can have greater than about 100 to about 3000 amino acid residues, preferably greater than 400 to about 3000 amino acid residues, in which no three contiguous amino acids in the sequence are identical amino acid types unless the amino acid is serine, in which case no more than three contiguous amino acids are serine residues. In the foregoing embodiment, the XTEN sequence would be substantially non-repetitive.

The degree of repetitiveness of a polypeptide or a gene can be measured by computer programs or algorithms or by other means known in the art. Repetitiveness in a polypeptide sequence can, for example, be assessed by determining the number of times shorter sequences of a given length occur within the polypeptide. For example, a polypeptide of 200 amino acid residues has 192 overlapping 9-amino acid sequences (or 9-mer “frames”) and 198 3-mer frames, but the number of unique 9-mer or 3-mer sequences will depend on the amount of repetitiveness within the sequence. A score can be generated (hereinafter “subsequence score”) that is reflective of the degree of repetitiveness of the subsequences in the overall polypeptide sequence. In the context of the present invention, “subsequence score” means the sum of occurrences of each unique 3-mer frame across a 200 consecutive amino acid sequence of the polypeptide divided by the absolute number of unique 3-mer subsequences within the 200 amino acid sequence. In some embodiments, the present invention provides BPXTEN each comprising XTEN in which the XTEN can have a subsequence score less than 12, more preferably less than 10, more preferably less than 9, more preferably less than 8, more preferably less than 7, more preferably less than 6, and most preferably less than 5. In the embodiments hereinabove described in this paragraph, an XTEN with a subsequence score less than about 10 (e.g., 9, 8, 7, etc.) would be “substantially non-repetitive.”

The non-repetitive characteristic of XTEN can impart to fusion proteins with BP(s) a greater degree of solubility and less tendency to aggregate compared to polypeptides having repetitive sequences. These properties can facilitate the formulation of XTEN-comprising pharmaceutical preparations containing extremely high drug concentrations, in some cases exceeding 100 mg/ml.

Furthermore, the XTEN polypeptide sequences of the embodiments are designed to have a low degree of internal repetitiveness in order to reduce or substantially eliminate immunogenicity when administered to a mammal. Polypeptide sequences composed of short, repeated motifs largely limited to three amino acids, such as glycine, serine and glutamate, may result in relatively high antibody titers when administered to a mammal despite the absence of predicted T-cell epitopes in these sequences. This may be caused by the repetitive nature of polypeptides, as it has been shown that immunogens with repeated epitopes, including protein aggregates, cross-linked immunogens, and repetitive carbohydrates are highly immunogenic and can, for example, result in the cross-linking of B-cell receptors causing B-cell activation. (Johansson, J., et al. (2007) Vaccine, 25:1676-82; Yankai, Z., et al. (2006) Biochem Biophys Res Commun, 345:1365-71; Hsu, C. T., et al. (2000) Cancer Res, 60:3701-5); Bachmann M F, et al. Eur J Immunol. (1995) 25(12):3445-3451).

### Exemplary Sequence Motifs

The present invention encompasses XTEN that can comprise multiple units of shorter sequences, or motifs, in which the amino acid sequences of the motifs are non-repetitive. In designing XTEN sequences, it was discovered that the non-repetitive criterion may be met despite the use of a “building block” approach using a library of sequence motifs that are multimerized to create the XTEN sequences. Thus, while an XTEN sequence may consist of multiple units of as few as four different types of sequence motifs, because the motifs themselves generally consist of non-repetitive amino acid sequences, the overall XTEN sequence is rendered substantially non-repetitive.

In one embodiment, XTEN can have a non-repetitive sequence of greater than about 100 to about 3000 amino acid residues, preferably greater than 400 to about 3000 residues, wherein at least about 80%, or at least about 85%, or at least about 90%, or at least about 95%, or at least about 97%, or about 100% of the XTEN sequence consists of non-overlapping sequence motifs, wherein each of the motifs has about 9 to 36 amino acid residues. In other embodiments, at least about 80%, or at least about 85%, or at least about 90%, or at least about 95%, or at least about 97%, or about 100% of the XTEN sequence consists of non-overlapping sequence motifs wherein each of the motifs has 9 to 14 amino acid residues. In still other embodiments, at least about 80%, or at least about 85%, or at least about 90%, or at least about 95%, or at least about 97%, or about 100% of the XTEN sequence component consists of non-overlapping sequence motifs wherein each of the motifs has 12 amino acid residues. In these embodiments, it is preferred that the sequence motifs be composed mainly of small hydrophilic amino acids, such that the overall sequence has an unstructured, flexible characteristic. Examples of amino acids that can be included in XTEN, are, e.g., arginine, lysine, threonine, alanine, asparagine, glutamine, aspartate, glutamate, serine, and glycine. As a result of testing variables such as codon optimization, assembly polynucleotides encoding sequence motifs, expression of protein, charge distribution and solubility of expressed protein, and secondary and tertiary structure, it was discovered that XTEN compositions with enhanced characteristics mainly include glycine (G), alanine (A), serine (S), threonine (T), glutamate (E) and proline (P) residues wherein the sequences are designed to be substantially non-repetitive. In a preferred embodiment, XTEN sequences have predominately four to six types of amino acids selected from glycine (G), alanine (A), serine (S), threonine (T), glutamate (E) or proline (P) that are arranged in a substantially non-repetitive sequence that is greater than about 100 to about 3000 amino acid residues, preferably greater than 400 to about 3000 residues in length. In some embodiments, XTEN can have sequences of greater than about 100 to about 3000 amino acid residues, preferably greater than 400 to about 3000 residues, wherein at least about 80% of the sequence consists of non-overlapping sequence motifs wherein each of the motifs has 9 to 36 amino acid residues wherein each of the motifs consists of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 9 to 36 amino acid residues wherein the motifs consist of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 12 amino acid residues consisting of 4 to 6 types of amino acids selected from glycine (G), alanine (A), serine (S), threonine (T), glutamate (E) and proline (P), and wherein the content of any one amino acid type in the full-length XTEN does not exceed 30%. In yet other embodiments, at least about 90%, or about 91%, or about 92%, or about 93%, or about 94%, or about 95%, or about 96%, or about 97%, or about 98%, or about 99%, to about 100% of the XTEN sequence consists of non-overlapping sequence motifs wherein each of the motifs has 12 amino acid residues consisting of glycine (G), alanine (A), serine (S), threonine (T), glutamate (E) and proline (P), and wherein in the content of any one amino acid type in the full-length XTEN does not exceed 30%.

In still other embodiments, XTENs comprise non-repetitive sequences of greater than about 100 to about 3000 amino acid residues, preferably greater than 400 to about 3000 amino acid residues wherein at least about 80%, or at least about 90%, or about 91%, or about 92%, or about 93%, or about 94%, or about 95%, or about 96%, or about 97%, or about 98%, or about 99% of the sequence consists of non-overlapping sequence motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one sequence motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of glycine (G), alanine (A), serine (S), threonine (T), glutamate (E) and proline (P), and wherein the sequence of any two contiguous amino acid residues in any one sequence motif is not repeated more than twice in the sequence motif. In yet other embodiments, XTENs consist of 12 amino acid sequence motifs wherein the amino acids are selected from glycine (G), alanine (A), serine (S), threonine (T), glutamate (E) and proline (P), and wherein the sequence of any two contiguous amino acid residues in any one sequence motif is not repeated more than twice in the sequence motif, and wherein the content of any one amino acid type in the full-length XTEN does not exceed 30%. In the foregoing embodiments hereinabove described in this paragraph, the XTEN sequences would be substantially non-repetitive.

In some cases, the invention provides compositions comprising a non-repetitive XTEN sequence of greater than about 100 to about 3000 amino acid residues, preferably greater than 400 to about 3000 residues, wherein at least about 80%, or at least about 90%, or about 91%, or about 92%, or about 93%, or about 94%, or about 95%, or about 96%, or about 97%, or about 98%, or about 99% to about 100% of the sequence consists of multiple units of two or more non-overlapping sequence motifs selected from the amino acid sequences of Table 1. In some cases, the XTEN comprises non-overlapping sequence motifs in which about 80%, or at least about 90%, or about 91%, or about 92%, or about 93%, or about 94%, or about 95%, or about 96%, or about 97%, or about 98%, or about 99% to about 100% of the sequence consists of two or more non-overlapping sequences selected from a single motif family of Table 1, resulting in a “family” sequence in which the overall sequence remains substantially non-repetitive. Accordingly, in these embodiments, an XTEN sequence can comprise multiple units of non-overlapping sequence motifs of the AD motif family, or the AE motif family, or the AF motif family, or the AG motif family, or the AM motif family, or the AQ motif family, or the BC family, or the BD family of sequences of Table 1. In other cases, the XTEN comprises motif sequences from two or more of the motif families of Table 1.

In some embodiments, where the composition of this disclosure (for example, a fusion protein) comprises an extended recombinant polypeptide (XTEN), the XTEN can be characterized in that: (i). it comprises at least 12 amino acids; (ii). at least 90%, 91%, 92%, 93%, 94%, 95%, 96%, 97%, 98%, 99% or 100% of the amino acid residues of the XTEN sequence are selected from glycine (G), alanine (A), serine (S), threonine (T), glutamate (E) and proline (P); and (iii). it has 4-6 different amino acids selected from G, A, S, T, E and P. In some embodiments, the XTEN sequence can consist of multiple non-overlapping sequence motifs, wherein the sequence motifs are (e.g., each independently) selected from the sequence motifs of Tables 2a-2b. In some embodiments, the XTEN can have from 40 to 3,000 amino acids, or from 100 to 3,000 amino acids. The XTEN can (e.g., each independently) have at least (about) 40, at least (about) 50, at least (about) 100, at least (about) 150, at least (about) 200, at least (about) 300, at least (about) 400, at least (about) 500, at least (about) 600, at least (about) 700, at least (about) 800, at least (about) 900, at least (about) 1,000 amino acids, at least (about) 1,500 amino acids, at least (about) 2,000 amino acids, at least (about) 2,500 amino acids, at least (about) 3,000 amino acids, or a range between any of the foregoing. In some embodiments, the XTEN can have at least 90%, at least 91%, at least 92%, at least 93%, at least 94%, at least 95%, at least 96%, at least 97%, at least 98%, or at least 99%, or 100% sequence identity to a sequence set forth in Tables 2a-2b.

TABLE 1
XTEN Sequence Motifs of 12 Amino Acids and Motif
Families
*Denotes individual motif sequences that, when
used together in various permutations, results
in a “family sequence”

| Motif Family* | SEQ ID NO: | Motif Sequence |
|---|---|---|
| AD | 182 | GESPGGSSGSES |
| AD | 183 | GSEGSSGPGESS |
| AD | 184 | GSSESGSSEGGP |
| AD | 185 | GSGGEPSESGSS |
| AE, AM | 186 | GSPAGSPTSTEE |
| AE, AM, AQ | 187 | GSEPATSGSETP |
| AE, AM, AQ | 188 | GTSESATPESGP |
| AE, AM, AQ | 189 | GTSTEPSEGSAP |
| AF, AM | 190 | GSTSESPSGTAP |
| AF, AM | 191 | GTSTPESGSASP |
| AF, AM | 192 | GTSPSGESSTAP |
| AF, AM | 193 | GSTSSTAESPGP |
| AG, AM | 194 | GTPGSGTASSSP |
| AG, AM | 195 | GSSTPSGATGSP |
| AG, AM | 196 | GSSPSASTGTGP |
| AG, AM | 197 | GASPGTSSTGSP |
| AQ | 198 | GEPAGSPTSTSE |
| AQ | 199 | GTGEPSSTPASE |
| AQ | 200 | GSGPSTESAPTE |
| AQ | 201 | GSETPSGPSETA |
| AQ | 202 | GPSETSTSEPGA |
| AQ | 203 | GSPSEPTEGTSA |
| BC | 881 | GSGASEPTSTEP |
| BC | 882 | GSEPATSGTEPS |
| BC | 883 | GTSEPSTSEPGA |
| BC | 884 | GTSTEPSEPGSA |
| BD | 885 | GSTAGSETSTEA |
| BD | 886 | GSETATSGSETA |
| BD | 887 | GTSESATSESGA |
| BD | 888 | GTSTEASEGSAS |

In those embodiments wherein the XTEN component of the BPXTEN fusion protein has less than 100% of its amino acids consisting of four to six amino acid selected from glycine (G), alanine (A), serine (S), threonine (T), glutamate (E) and proline (P), or less than 100% of the sequence consisting of the sequence motifs of Tables 1 or less than 100% sequence identity with an XTEN from Tables 2a-2b, the other amino acid residues can be selected from any other of the 14 natural L-amino acids. The other amino acids may be interspersed throughout the XTEN sequence, may be located within or between the sequence motifs, or may be concentrated in one or more short stretches of the XTEN sequence. In such cases where the XTEN component of the BPXTEN comprises amino acids other than glycine (G), alanine (A), serine (S), threonine (T), glutamate (E) and proline (P), it is preferred that the amino acids not be hydrophobic residues and should not substantially confer secondary structure of the XTEN component. Thus, in a preferred embodiment of the foregoing, the XTEN component of the BPXTEN fusion protein comprising other amino acids in addition to glycine (G), alanine (A), serine (S), threonine (T), glutamate (E) and proline (P) would have a sequence with less than 5% of the residues contributing to alpha-helices and beta-sheets as measured by Chou-Fasman algorithm and would have at least 90% random coil formation as measured by GOR algorithm.

### Length of Sequence

In a particular feature, the invention encompasses BPXTEN compositions comprising XTEN polypeptides with extended length sequences. The present invention makes use of the discovery that increasing the length of non-repetitive, unstructured polypeptides enhances the unstructured nature of the XTENs and the biological and pharmacokinetic properties of fusion proteins comprising the XTEN. As described more fully in the Examples, proportional increases in the length of the XTEN, even if created by a fixed repeat order of single family sequence motifs (e.g., the four AE motifs of Table 1), can result in a sequence with a higher percentage of random coil formation, as determined by GOR algorithm, compared to shorter XTEN lengths. In addition, it was discovered that increasing the length of the unstructured polypeptide fusion partner can, as described in the Examples, result in a fusion protein with a disproportional increase in terminal half-life compared to fusion proteins with unstructured polypeptide partners with shorter sequence lengths.

Non-limiting examples of XTEN contemplated for inclusion in the BPXTEN of the invention are presented in Tables 2a-2b. Accordingly, the invention provides BPXTEN compositions wherein the XTEN sequence length of the fusion protein(s) is greater than about 100 to about 3000 amino acid residues, and in some cases is greater than 400 to about 3000 amino acid residues, wherein the XTEN confers enhanced pharmacokinetic properties on the BPXTEN in comparison to payloads not linked to XTEN. In some cases, the XTEN sequences of the BPXTEN compositions of the present invention can be about 100, or about 144, or about 288, or about 401, or about 500, or about 600, or about 700, or about 800, or about 900, or about 1000, or about 1500, or about 2000, or about 2500 or up to about 3000 amino acid residues in length. In other cases, the XTEN sequences can be about 100 to 150, about 150 to 250, about 250 to 400, 401 to about 500, about 500 to 900, about 900 to 1500, about 1500 to 2000, or about 2000 to about 3000 amino acid residues in length. In one embodiment, the BPXTEN can comprise an XTEN sequence wherein the sequence exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a XTEN selected from Tables 2a-2b. In some cases, the XTEN sequence is designed for optimized expression as the N-terminal component of the BPXTEN. In one embodiment of the foregoing, the XTEN sequence has at least 90% sequence identity to the sequence of AE912 or AM923. In another embodiment of the foregoing, the XTEN has the N-terminal residues described in Examples 14-17.

In other cases, the BPXTEN fusion protein can comprise a first and a second XTEN sequence, wherein the cumulative total of the residues in the XTEN sequences is greater than about 400 to about 3000 amino acid residues. In embodiments of the foregoing, the BPXTEN fusion protein can comprise a first and a second XTEN sequence wherein the sequences each exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to at least a first or additionally a second XTEN selected from Tables 2a-2b. Examples where more than one XTEN is used in a BPXTEN composition include, but are not limited to constructs with an XTEN linked to both the N- and C-termini of at least one BP.

As described more fully below, the invention provides methods in which the BPXTEN is designed by selecting the length of the XTEN to confer a target half-life on a fusion protein administered to a subject. In some cases, the BPXTEN can be designed by selecting the length of the XTEN to confer a target masking effect on the biological polypeptide for administration to a subject. In general, longer XTEN lengths incorporated into the BPXTEN compositions result in longer half-life compared to shorter XTEN. However, in another embodiment, BPXTEN fusion proteins can be designed to comprise XTEN with a longer sequence length that is selected to confer slower rates of systemic absorption after subcutaneous or intramuscular administration to a subject. In such cases, the Cₘₐₓ is reduced in comparison to a comparable dose of a BP not linked to XTEN, thereby contributing to the ability to keep the BPXTEN within the therapeutic window for the composition. Thus, the XTEN confers the property of a depot to the administered BPXTEN, in addition to the other physical/chemical properties described herein.

TABLE 2A
Exemplary XTEN Polypeptides

| XTEN | SEQ ID | |
|---|---|---|
| Name | NO: | Amino Acid Sequence |
| AE144 | 204 | GSEPATSGSETPGTSESATPESGPGSEPATSGSETPGSPAGSPTSTEEGTSTEPS |
| | | EGSAPGSEPATSGSETPGSEPATSGSETPGSEPATSGSETPGTSTEPSEGSAPGT |
| | | SESATPESGPGSEPATSGSETPGTSTEPSEGSAP |
| AF144 | 205 | GTSTPESGSASPGTSPSGESSTAPGTSPSGESSTAPGSTSSTAESPGPGSTSESP |
| | | SGTAPGSTSSTAESPGPGTSPSGESSTAPGTSTPESGSASPGSTSSTAESPGPGT |
| | | SPSGESSTAPGTSPSGESSTAPGTSPSGESSTAP |
| AE288 | 206 | GTSESATPESGPGSEPATSGSETPGTSESATPESGPGSEPATSGSETPGTSESAT |
| | | PESGPGTSTEPSEGSAPGSPAGSPTSTEEGTSESATPESGPGSEPATSGSETPGT |
| | | SESATPESGPGSPAGSPTSTEEGSPAGSPTSTEEGTSTEPSEGSAPGTSESATPE |
| | | SGPGTSESATPESGPGTSESATPESGPGSEPATSGSETPGSEPATSGSETPGSPA |
| | | GSPTSTEEGTSTEPSEGSAPGTSTEPSEGSAPGSEPATSGSETPGTSESATPESG |
| | | PGTSTEPSEGSAP |
| AF504 | 207 | GASPGTSSTGSPGSSPSASTGTGPGSSPSASTGTGPGTPGSGTASSSPGSSTPSG |
| | | ATGSPGSXPSASTGTGPGASPGTSSTGSPGTPGSGTASSSPGSSTPSGATGSPGT |
| | | PGSGTASSSPGASPGTSSTGSPGASPGTSSTGSPGTPGSGTASSSPGSSTPSGAT |
| | | GSPGASPGTSSTGSPGTPGSGTASSSPGSSTPSGATGSPGSXPSASTGTGPGSSP |
| | | SASTGTGPGSSTPSGATGSPGSSTPSGATGSPGASPGTSSTGSPGASPGTSSTGS |
| | | PGASPGTSSTGSPGTPGSGTASSSPGASPGTSSTGSPGASPGTSSTGSPGASPGT |
| | | SSTGSPGSSPSASTGTGPGTPGSGTASSSPGASPGTSSTGSPGASPGTSSTGSPG |
| | | ASPGTSSTGSPGSSTPSGATGSPGSSTPSGATGSPGASPGTSSTGSPGTPGSGTA |
| | | SSSPGSSTPSGATGSPGSSTPSGATGSPGSSTPSGATGSPGSSPSASTGTGPGAS |
| | | PGTSSTGSP |
| AF540 | 208 | GSTSSTAESPGPGSTSSTAESPGPGSTSESPSGTAPGSTSSTAESPGPGSTSSTA |
| | | ESPGPGTSTPESGSASPGSTSESPSGTAPGTSPSGESSTAPGSTSESPSGTAPGS |
| | | TSESPSGTAPGTSPSGESSTAPGSTSESPSGTAPGSTSESPSGTAPGTSPSGESS |
| | | TAPGSTSESPSGTAPGSTSESPSGTAPGSTSESPSGTAPGTSTPESGSASPGSTS |
| | | ESPSGTAPGTSTPESGSASPGSTSSTAESPGPGSTSSTAESPGPGTSTPESGSAS |
| | | PGTSTPESGSASPGSTSESPSGTAPGTSTPESGSASPGTSTPESGSASPGSTSES |
| | | PSGTAPGSTSESPSGTAPGSTSESPSGTAPGSTSSTAESPGPGTSTPESGSASPG |
| | | TSTPESGSASPGSTSESPSGTAPGSTSESPSGTAPGTSTPESGSASPGSTSESPS |
| | | GTAPGSTSESPSGTAPGTSTPESGSASPGTSPSGESSTAPGSTSSTAESPGPGTS |
| | | PSGESSTAPGSTSSTAESPGPGTSTPESGSASPGSTSESPSGTAP |
| AD576 | 209 | GSSESGSSEGGPGSGGEPSESGSSGSSESGSSEGGPGSSESGSSEGGPGSSESGS |
| | | SEGGPGSSESGSSEGGPGSSESGSSEGGPGESPGGSSGSESGSEGSSGPGESSGS |
| | | SESGSSEGGPGSSESGSSEGGPGSSESGSSEGGPGSGGEPSESGSSGESPGGSSG |
| | | SESGESPGGSSGSESGSGGEPSESGSSGSSESGSSEGGPGSGGEPSESGSSGSGG |
| | | EPSESGSSGSEGSSGPGESSGESPGGSSGSESGSGGEPSESGSSGSGGEPSESGS |
| | | SGSGGEPSESGSSGSSESGSSEGGPGESPGGSSGSESGESPGGSSGSESGESPGG |
| | | SSGSESGESPGGSSGSESGESPGGSSGSESGSSESGSSEGGPGSGGEPSESGSSG |
| | | SEGSSGPGESSGSSESGSSEGGPGSGGEPSESGSSGSSESGSSEGGPGSGGEPSE |
| | | SGSSGESPGGSSGSESGESPGGSSGSESGSSESGSSEGGPGSGGEPSESGSSGSS |
| | | ESGSSEGGPGSGGEPSESGSSGSGGEPSESGSSGESPGGSSGSESGSEGSSGPGE |
| | | SSGSSESGSSEGGPGSEGSSGPGESS |
| AE576 | 210 | GSPAGSPTSTEEGTSESATPESGPGTSTEPSEGSAPGSPAGSPTSTEEGTSTEPS |
| | | EGSAPGTSTEPSEGSAPGTSESATPESGPGSEPATSGSETPGSEPATSGSETPGS |
| | | PAGSPTSTEEGTSESATPESGPGTSTEPSEGSAPGTSTEPSEGSAPGSPAGSPTS |
| | | TEEGTSTEPSEGSAPGTSTEPSEGSAPGTSESATPESGPGTSTEPSEGSAPGTSE |
| | | SATPESGPGSEPATSGSETPGTSTEPSEGSAPGTSTEPSEGSAPGTSESATPESG |
| | | PGTSESATPESGPGSPAGSPTSTEEGTSESATPESGPGSEPATSGSETPGTSESA |
| | | TPESGPGTSTEPSEGSAPGTSTEPSEGSAPGTSTEPSEGSAPGTSTEPSEGSAPG |
| | | TSTEPSEGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPESGPGTS |
| | | TEPSEGSAPGTSESATPESGPGSPAGSPTSTEEGSPAGSPTSTEEGSPAGSPTST |
| | | EEGTSESATPESGPGTSTEPSEGSAP |
| AF576 | 211 | GSTSSTAESPGPGSTSSTAESPGPGSTSESPSGTAPGSTSSTAESPGPGSTSSTA |
| | | ESPGPGTSTPESGSASPGSTSESPSGTAPGTSPSGESSTAPGSTSESPSGTAPGS |
| | | TSESPSGTAPGTSPSGESSTAPGSTSESPSGTAPGSTSESPSGTAPGTSPSGESS |
| | | TAPGSTSESPSGTAPGSTSESPSGTAPGSTSESPSGTAPGTSTPESGSASPGSTS |
| | | ESPSGTAPGTSTPESGSASPGSTSSTAESPGPGSTSSTAESPGPGTSTPESGSAS |
| | | PGTSTPESGSASPGSTSESPSGTAPGTSTPESGSASPGTSTPESGSASPGSTSES |
| | | PSGTAPGSTSESPSGTAPGSTSESPSGTAPGSTSSTAESPGPGTSTPESGSASPG |
| | | TSTPESGSASPGSTSESPSGTAPGSTSESPSGTAPGTSTPESGSASPGSTSESPS |
| | | GTAPGSTSESPSGTAPGTSTPESGSASPGTSPSGESSTAPGSTSSTAESPGPGTS |
| | | PSGESSTAPGSTSSTAESPGPGTSTPESGSASPGSTSESPSGTAPGSTSSTAESP |
| | | GPGTSTPESGSASPGTSTPESGSASP |
| AD836 | 212 | GSSESGSSEGGPGSSESGSSEGGPGESPGGSSGSESGSGGEPSESGSSGESPGGS |
| | | SGSESGESPGGSSGSESGSSESGSSEGGPGSSESGSSEGGPGSSESGSSEGGPGE |
| | | SPGGSSGSESGESPGGSSGSESGESPGGSSGSESGSSESGSSEGGPGSSESGSSE |
| | | GGPGSSESGSSEGGPGSSESGSSEGGPGSSESGSSEGGPGSSESGSSEGGPGSGG |
| | | EPSESGSSGESPGGSSGSESGESPGGSSGSESGSGGEPSESGSSGSEGSSGPGES |
| | | SGSSESGSSEGGPGSGGEPSESGSSGSEGSSGPGESSGSSESGSSEGGPGSGGEP |
| | | SESGSSGESPGGSSGSESGSGGEPSESGSSGSGGEPSESGSSGSSESGSSEGGPG |
| | | SGGEPSESGSSGSGGEPSESGSSGSEGSSGPGESSGESPGGSSGSESGSEGSSGP |
| | | GESSGSEGSSGPGESSGSGGEPSESGSSGSSESGSSEGGPGSSESGSSEGGPGES |
| | | PGGSSGSESGSGGEPSESGSSGSEGSSGPGESSGESPGGSSGSESGSEGSSGPGS |
| | | SESGSSEGGPGSGGEPSESGSSGSEGSSGPGESSGSEGSSGPGESSGSEGSSGPG |
| | | ESSGSGGEPSESGSSGSGGEPSESGSSGESPGGSSGSESGESPGGSSGSESGSGG |
| | | EPSESGSSGSEGSSGPGESSGESPGGSSGSESGSSESGSSEGGPGSSESGSSEGG |
| | | PGSSESGSSEGGPGSGGEPSESGSSGSSESGSSEGGPGESPGGSSGSESGSGGEP |
| | | SESGSSGSSESGSSEGGPGESPGGSSGSESGSGGEPSESGSSGESPGGSSGSESG |
| | | SGGEPSESGSS |
| AE864 | 213 | GSPAGSPTSTEEGTSESATPESGPGTSTEPSEGSAPGSPAGSPTSTEEGTSTEPS |
| | | EGSAPGTSTEPSEGSAPGTSESATPESGPGSEPATSGSETPGSEPATSGSETPGS |
| | | PAGSPTSTEEGTSESATPESGPGTSTEPSEGSAPGTSTEPSEGSAPGSPAGSPTS |
| | | TEEGTSTEPSEGSAPGTSTEPSEGSAPGTSESATPESGPGTSTEPSEGSAPGTSE |
| | | SATPESGPGSEPATSGSETPGTSTEPSEGSAPGTSTEPSEGSAPGTSESATPESG |
| | | PGTSESATPESGPGSPAGSPTSTEEGTSESATPESGPGSEPATSGSETPGTSESA |
| | | TPESGPGTSTEPSEGSAPGTSTEPSEGSAPGTSTEPSEGSAPGTSTEPSEGSAPG |
| | | TSTEPSEGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPESGPGTS |
| | | TEPSEGSAPGTSESATPESGPGSPAGSPTSTEEGSPAGSPTSTEEGSPAGSPTST |
| | | EEGTSESATPESGPGTSTEPSEGSAPGTSESATPESGPGSEPATSGSETPGTSES |
| | | ATPESGPGSEPATSGSETPGTSESATPESGPGTSTEPSEGSAPGSPAGSPTSTEE |
| | | GTSESATPESGPGSEPATSGSETPGTSESATPESGPGSPAGSPTSTEEGSPAGSP |
| | | TSTEEGTSTEPSEGSAPGTSESATPESGPGTSESATPESGPGTSESATPESGPGS |
| | | EPATSGSETPGSEPATSGSETPGSPAGSPTSTEEGTSTEPSEGSAPGTSTEPSEG |
| | | SAPGSEPATSGSETPGTSESATPESGPGTSTEPSEGSAP |
| AF864 | 214 | GSTSESPSGTAPGTSPSGESSTAPGSTSESPSGTAPGSTSESPSGTAPGTSTPES |
| | | GSASPGTSTPESGSASPGSTSESPSGTAPGSTSESPSGTAPGTSPSGESSTAPGS |
| | | TSESPSGTAPGTSPSGESSTAPGTSPSGESSTAPGSTSSTAESPGPGTSPSGESS |
| | | TAPGTSPSGESSTAPGSTSSTAESPGPGTSTPESGSASPGTSTPESGSASPGSTS |
| | | ESPSGTAPGSTSESPSGTAPGTSTPESGSASPGSTSSTAESPGPGTSTPESGSAS |
| | | PGSTSESPSGTAPGTSPSGESSTAPGSTSSTAESPGPGTSPSGESSTAPGTSTPE |
| | | SGSASPGSTSSTAESPGPGSTSSTAESPGPGSTSSTAESPGPGSTSSTAESPGPG |
| | | TSPSGESSTAPGSTSESPSGTAPGSTSESPSGTAPGTSTPESGPXXXGASASGAP |
| | | STXXXXSESPSGTAPGSTSESPSGTAPGSTSESPSGTAPGSTSESPSGTAPGSTS |
| | | ESPSGTAPGSTSESPSGTAPGTSTPESGSASPGTSPSGESSTAPGTSPSGESSTA |
| | | PGSTSSTAESPGPGTSPSGESSTAPGTSTPESGSASPGSTSESPSGTAPGSTSES |
| | | PSGTAPGTSPSGESSTAPGSTSESPSGTAPGTSTPESGSASPGTSTPESGSASPG |
| | | STSESPSGTAPGTSTPESGSASPGSTSSTAESPGPGSTSESPSGTAPGSTSESPS |
| | | GTAPGTSPSGESSTAPGSTSSTAESPGPGTSPSGESSTAPGTSTPESGSASPGTS |
| | | PSGESSTAPGTSPSGESSTAPGTSPSGESSTAPGSTSSTAESPGPGSTSSTAESP |
| | | GPGTSPSGESSTAPGSSPSASTGTGPGSSTPSGATGSPGSSTPSGATGSP |
| AG864 | 215 | GASPGTSSTGSPGSSPSASTGTGPGSSPSASTGTGPGTPGSGTASSSPGSSTPSG |
| | | ATGSPGSSPSASTGTGPGASPGTSSTGSPGTPGSGTASSSPGSSTPSGATGSPGT |
| | | PGSGTASSSPGASPGTSSTGSPGASPGTSSTGSPGTPGSGTASSSPGSSTPSGAT |
| | | GSPGASPGTSSTGSPGTPGSGTASSSPGSSTPSGATGSPGSSPSASTGTGPGSSP |
| | | SASTGTGPGSSTPSGATGSPGSSTPSGATGSPGASPGTSSTGSPGASPGTSSTGS |
| | | PGASPGTSSTGSPGTPGSGTASSSPGASPGTSSTGSPGASPGTSSTGSPGASPGT |
| | | SSTGSPGSSPSASTGTGPGTPGSGTASSSPGASPGTSSTGSPGASPGTSSTGSPG |
| | | ASPGTSSTGSPGSSTPSGATGSPGSSTPSGATGSPGASPGTSSTGSPGTPGSGTA |
| | | SSSPGSSTPSGATGSPGSSTPSGATGSPGSSTPSGATGSPGSSPSASTGTGPGAS |
| | | PGTSSTGSPGASPGTSSTGSPGTPGSGTASSSPGASPGTSSTGSPGASPGTSSTG |
| | | SPGASPGTSSTGSPGASPGTSSTGSPGTPGSGTASSSPGSSTPSGATGSPGTPGS |
| | | GTASSSPGSSTPSGATGSPGTPGSGTASSSPGSSTPSGATGSPGSSTPSGATGSP |
| | | GSSPSASTGTGPGSSPSASTGTGPGASPGTSSTGSPGTPGSGTASSSPGSSTPSG |
| | | ATGSPGSSPSASTGTGPGSSPSASTGTGPGASPGTSSTGSPGASPGTSSTGSPGS |
| | | STPSGATGSPGSSPSASTGTGPGASPGTSSTGSPGSSPSASTGTGPGTPGSGTAS |
| | | SSPGSSTPSGATGSPGSSTPSGATGSPGASPGTSSTGSP |
| AM875 | 216 | GTSTEPSEGSAPGSEPATSGSETPGSPAGSPTSTEEGSTSSTAESPGPGTSTPES |
| | | GSASPGSTSESPSGTAPGSTSESPSGTAPGTSTPESGSASPGTSTPESGSASPGS |
| | | EPATSGSETPGTSESATPESGPGSPAGSPTSTEEGTSTEPSEGSAPGTSESATPE |
| | | SGPGTSTEPSEGSAPGTSTEPSEGSAPGSPAGSPTSTEEGTSTEPSEGSAPGTST |
| | | EPSEGSAPGTSESATPESGPGTSESATPESGPGTSTEPSEGSAPGTSTEPSEGSA |
| | | PGTSESATPESGPGTSTEPSEGSAPGSEPATSGSETPGSPAGSPTSTEEGSSTPS |
| | | GATGSPGTPGSGTASSSPGSSTPSGATGSPGTSTEPSEGSAPGTSTEPSEGSAPG |
| | | SEPATSGSETPGSPAGSPTSTEEGSPAGSPTSTEEGTSTEPSEGSAPGASASGAP |
| | | STGGTSESATPESGPGSPAGSPTSTEEGSPAGSPTSTEEGSTSSTAESPGPGSTS |
| | | ESPSGTAPGTSPSGESSTAPGTPGSGTASSSPGSSTPSGATGSPGSSPSASTGTG |
| | | PGSEPATSGSETPGTSESATPESGPGSEPATSGSETPGSTSSTAESPGPGSTSST |
| | | AESPGPGTSPSGESSTAPGSEPATSGSETPGSEPATSGSETPGTSTEPSEGSAPG |
| | | STSSTAESPGPGTSTPESGSASPGSTSESPSGTAPGTSTEPSEGSAPGTSTEPSE |
| | | GSAPGTSTEPSEGSAPGSSTPSGATGSPGSSPSASTGTGPGASPGTSSTGSPGSE |
| | | PATSGSETPGTSESATPESGPGSPAGSPTSTEEGSSTPSGATGSPGSSPSASTGT |
| | | GPGASPGTSSTGSPGTSESATPESGPGTSTEPSEGSAPGTSTEPSEGSAP |
| AE912 | 217 | MAEPAGSPTSTEEGTPGSGTASSSPGSSTPSGATGSPGASPGTSSTGSPGSPAGS |
| | | PTSTEEGTSESATPESGPGTSTEPSEGSAPGSPAGSPTSTEEGTSTEPSEGSAPG |
| | | TSTEPSEGSAPGTSESATPESGPGSEPATSGSETPGSEPATSGSETPGSPAGSPT |
| | | STEEGTSESATPESGPGTSTEPSEGSAPGTSTEPSEGSAPGSPAGSPTSTEEGTS |
| | | TEPSEGSAPGTSTEPSEGSAPGTSESATPESGPGTSTEPSEGSAPGTSESATPES |
| | | GPGSEPATSGSETPGTSTEPSEGSAPGTSTEPSEGSAPGTSESATPESGPGTSES |
| | | ATPESGPGSPAGSPTSTEEGTSESATPESGPGSEPATSGSETPGTSESATPESGP |
| | | GTSTEPSEGSAPGTSTEPSEGSAPGTSTEPSEGSAPGTSTEPSEGSAPGTSTEPS |
| | | EGSAPGTSTEPSEGSAPGSPAGSPTSTEEGTSTEPSEGSAPGTSESATPESGPGS |
| | | EPATSGSETPGTSESATPESGPGSEPATSGSETPGTSESATPESGPGTSTEPSEG |
| | | SAPGTSESATPESGPGSPAGSPTSTEEGSPAGSPTSTEEGSPAGSPTSTEEGTSE |
| | | SATPESGPGTSTEPSEGSAPGTSESATPESGPGSEPATSGSETPGTSESATPESG |
| | | PGSEPATSGSETPGTSESATPESGPGTSTEPSEGSAPGSPAGSPTSTEEGTSESA |
| | | TPESGPGSEPATSGSETPGTSESATPESGPGSPAGSPTSTEEGSPAGSPTSTEEG |
| | | TSTEPSEGSAPGTSESATPESGPGTSESATPESGPGTSESATPESGPGSEPATSG |
| | | SETPGSEPATSGSETPGSPAGSPTSTEEGTSTEPSEGSAPGTSTEPSEGSAPGSE |
| | | PATSGSETPGTSESATPESGPGTSTEPSEGSAP |
| AM923 | 218 | MAEPAGSPTSTEEGASPGTSSTGSPGSSTPSGATGSPGSSTPSGATGSPGTSTEP |
| | | SEGSAPGSEPATSGSETPGSPAGSPTSTEEGSTSSTAESPGPGTSTPESGSASPG |
| | | STSESPSGTAPGSTSESPSGTAPGTSTPESGSASPGTSTPESGSASPGSEPATSG |
| | | SETPGTSESATPESGPGSPAGSPTSTEEGTSTEPSEGSAPGTSESATPESGPGTS |
| | | TEPSEGSAPGTSTEPSEGSAPGSPAGSPTSTEEGTSTEPSEGSAPGTSTEPSEGS |
| | | APGTSESATPESGPGTSESATPESGPGTSTEPSEGSAPGTSTEPSEGSAPGTSES |
| | | ATPESGPGTSTEPSEGSAPGSEPATSGSETPGSPAGSPTSTEEGSSTPSGATGSP |
| | | GTPGSGTASSSPGSSTPSGATGSPGTSTEPSEGSAPGTSTEPSEGSAPGSEPATS |
| | | GSETPGSPAGSPTSTEEGSPAGSPTSTEEGTSTEPSEGSAPGASASGAPSTGGTS |
| | | ESATPESGPGSPAGSPTSTEEGSPAGSPTSTEEGSTSSTAESPGPGSTSESPSGT |
| | | APGTSPSGESSTAPGTPGSGTASSSPGSSTPSGATGSPGSSPSASTGTGPGSEPA |
| | | TSGSETPGTSESATPESGPGSEPATSGSETPGSTSSTAESPGPGSTSSTAESPGP |
| | | GTSPSGESSTAPGSEPATSGSETPGSEPATSGSETPGTSTEPSEGSAPGSTSSTA |
| | | ESPGPGTSTPESGSASPGSTSESPSGTAPGTSTEPSEGSAPGTSTEPSEGSAPGT |
| | | STEPSEGSAPGSSTPSGATGSPGSSPSASTGTGPGASPGTSSTGSPGSEPATSGS |
| | | ETPGTSESATPESGPGSPAGSPTSTEEGSSTPSGATGSPGSSPSASTGTGPGASP |
| | | GTSSTGSPGTSESATPESGPGTSTEPSEGSAPGTSTEPSEGSAP |
| AM1296 | 219 | GTSTEPSEGSAPGSEPATSGSETPGSPAGSPTSTEEGSTSSTAESPGPGTSTPES |
| | | GSASPGSTSESPSGTAPGSTSESPSGTAPGTSTPESGSASPGTSTPESGSASPGS |
| | | EPATSGSETPGTSESATPESGPGSPAGSPTSTEEGTSTEPSEGSAPGTSESATPE |
| | | SGPGTSTEPSEGSAPGTSTEPSEGSAPGSPAGSPTSTEEGTSTEPSEGSAPGTST |
| | | EPSEGSAPGTSESATPESGPGTSESATPESGPGTSTEPSEGSAPGTSTEPSEGSA |
| | | PGTSESATPESGPGTSTEPSEGSAPGSEPATSGSETPGSPAGSPTSTEEGSSTPS |
| | | GATGSPGTPGSGTASSSPGSSTPSGATGSPGTSTEPSEGSAPGTSTEPSEGSAPG |
| | | SEPATSGSETPGSPAGSPTSTEEGSPAGSPTSTEEGTSTEPSEGSAPGPEPTGPA |
| | | PSGGSEPATSGSETPGTSESATPESGPGSPAGSPTSTEEGTSESATPESGPGSPA |
| | | GSPTSTEEGSPAGSPTSTEEGTSESATPESGPGSPAGSPTSTEEGSPAGSPTSTE |
| | | EGSTSSTAESPGPGSTSESPSGTAPGTSPSGESSTAPGSTSESPSGTAPGSTSES |
| | | PSGTAPGTSPSGESSTAPGTSTEPSEGSAPGTSESATPESGPGTSESATPESGPG |
| | | SEPATSGSETPGTSESATPESGPGTSESATPESGPGTSTEPSEGSAPGTSESATP |
| | | ESGPGTSTEPSEGSAPGTSPSGESSTAPGTSPSGESSTAPGTSPSGESSTAPGTS |
| | | TEPSEGSAPGSPAGSPTSTEEGTSTEPSEGSAPGSSPSASTGTGPGSSTPSGATG |
| | | SPGSSTPSGATGSPGSSTPSGATGSPGSSTPSGATGSPGASPGTSSTGSPGASAS |
| | | GAPSTGGTSPSGESSTAPGSTSSTAESPGPGTSPSGESSTAPGTSESATPESGPG |
| | | TSTEPSEGSAPGTSTEPSEGSAPGSSPSASTGTGPGSSTPSGATGSPGASPGTSS |
| | | TGSPGTSTPESGSASPGTSPSGESSTAPGTSPSGESSTAPGTSESATPESGPGSE |
| | | PATSGSETPGTSTEPSEGSAPGSTSESPSGTAPGSTSESPSGTAPGTSTPESGSA |
| | | SPGSPAGSPTSTEEGTSESATPESGPGTSTEPSEGSAPGSPAGSPTSTEEGTSES |
| | | ATPESGPGSEPATSGSETPGSSTPSGATGSPGASPGTSSTGSPGSSTPSGATGSP |
| | | GSTSESPSGTAPGTSPSGESSTAPGSTSSTAESPGPGSSTPSGATGSPGASPGTS |
| | | STGSPGTPGSGTASSSPGSPAGSPTSTEEGSPAGSPTSTEEGTSTEPSEGSAP |
| BC 864 | 220 | GTSTEPSEPGSAGTSTEPSEPGSAGSEPATSGTEPSGSGASEPTSTEPGSEPATS |
| | | GTEPSGSEPATSGTEPSGSEPATSGTEPSGSGASEPTSTEPGTSTEPSEPGSAGS |
| | | EPATSGTEPSGTSTEPSEPGSAGSEPATSGTEPSGSEPATSGTEPSGTSTEPSEP |
| | | GSAGTSTEPSEPGSAGSEPATSGTEPSGSEPATSGTEPSGTSEPSTSEPGAGSGA |
| | | SEPTSTEPGTSEPSTSEPGAGSEPATSGTEPSGSEPATSGTEPSGTSTEPSEPGS |
| | | AGTSTEPSEPGSAGSGASEPTSTEPGSEPATSGTEPSGSEPATSGTEPSGSEPAT |
| | | SGTEPSGSEPATSGTEPSGTSTEPSEPGSAGSEPATSGTEPSGSGASEPTSTEPG |
| | | TSTEPSEPGSAGSEPATSGTEPSGSGASEPTSTEPGTSTEPSEPGSAGSGASEPT |
| | | STEPGSEPATSGTEPSGSGASEPTSTEPGSEPATSGTEPSGSGASEPTSTEPGTS |
| | | TEPSEPGSAGSEPATSGTEPSGSGASEPTSTEPGTSTEPSEPGSAGSEPATSGTE |
| | | PSGTSTEPSEPGSAGSEPATSGTEPSGTSTEPSEPGSAGTSTEPSEPGSAGTSTE |
| | | PSEPGSAGTSTEPSEPGSAGTSTEPSEPGSAGTSTEPSEPGSAGTSEPSTSEPGA |
| | | GSGASEPTSTEPGTSTEPSEPGSAGTSTEPSEPGSAGTSTEPSEPGSAGSEPATS |
| | | GTEPSGSGASEPTSTEPGSEPATSGTEPSGSEPATSGTEPSGSEPATSGTEPSGS |
| | | EPATSGTEPSGTSEPSTSEPGAGSEPATSGTEPSGSGASEPTSTEPGTSTEPSEP |
| | | GSAGSEPATSGTEPSGSGASEPTSTEPGTSTEPSEPGSA |
| BD864 | 221 | GSETATSGSETAGTSESATSESGAGSTAGSETSTEAGTSESATSESGAGSETATS |
| | | GSETAGSETATSGSETAGTSTEASEGSASGTSTEASEGSASGTSESATSESGAGS |
| | | ETATSGSETAGTSTEASEGSASGSTAGSETSTEAGTSESATSESGAGTSESATSE |
| | | SGAGSETATSGSETAGTSESATSESGAGTSTEASEGSASGSETATSGSETAGSET |
| | | ATSGSETAGTSTEASEGSASGSTAGSETSTEAGTSESATSESGAGTSTEASEGSA |
| | | SGSETATSGSETAGSTAGSETSTEAGSTAGSETSTEAGSETATSGSETAGTSESA |
| | | TSESGAGTSESATSESGAGSETATSGSETAGTSESATSESGAGTSESATSESGAG |
| | | SETATSGSETAGSETATSGSETAGTSTEASEGSASGSTAGSETSTEAGSETATSG |
| | | SETAGTSESATSESGAGSTAGSETSTEAGSTAGSETSTEAGSTAGSETSTEAGTS |
| | | TEASEGSASGSTAGSETSTEAGSTAGSETSTEAGTSTEASEGSASGSTAGSETST |
| | | EAGSETATSGSETAGTSTEASEGSASGTSESATSESGAGSETATSGSETAGTSES |
| | | ATSESGAGTSESATSESGAGSETATSGSETAGTSESATSESGAGSETATSGSETA |
| | | GTSTEASEGSASGTSTEASEGSASGSTAGSETSTEAGSTAGSETSTEAGSETATS |
| | | GSETAGTSESATSESGAGTSESATSESGAGSETATSGSETAGSETATSGSETAGS |
| | | ETATSGSETAGTSTEASEGSASGTSESATSESGAGSETATSGSETAGSETATSGS |
| | | ETAGTSESATSESGAGTSESATSESGAGSETATSGSETA |

TABLE 2B
Exemplary XTEN polypeptides

| SEQ ID | Exemplary | |
|---|---|---|
| NO. | Use | Amino Acid Sequence |
| 889 | C-terminal | PGSPAGSPTSTEEGTSESATPESGPGTSTEPSEGSAPGSPAGSPTSTEEGTST |
| (previously | XTEN | EPSEGSAPGTSTEPSEGSAPGTSESATPESGPGSEPATSGSETPGSEPATSGS |
| 8001) | | ETPGSPAGSPTSTEEGTSESATPESGPGTSTEPSEGSAPGTSTEPSEGSAPGS |
| | | PAGSPTSTEEGTSTEPSEGSAPGTSTEPSEGSAPGTSESATPESGPGTSTEPS |
| | | EGSAPGTSESATPESGPGSEPATSGSETPGTSTEPSEGSAPGTSTEPSEGSAP |
| | | GTSESATPESGPGTSESATPESGPGSPAGSPTSTEEGTSESATPESGPGSEPA |
| | | TSGSETPGTSESATPESGPGTSTEPSEGSAPGTSTEPSEGSAPGTSTEPSEGS |
| | | APGTSTEPSEGSAPGTSTEPSEGSAPGTSTEPSEGSAPGSPAGSPTSTEEGTS |
| | | TEPSEGSAPGTSESATPESGPGSEPATSGSETPGTSESATPESGPGSEPATSG |
| | | SETPGTSESATPESGPGTSTEPSEGSAPGTSESATPESGPGSPAGSPTSTEEG |
| | | SPAGSPTSTEEGSPAGSPTSTEEGTSESATPESGPGTSTEPSEGSAPGTSESA |
| | | TPESGPGSEPATSGSETPGTSESATPESGPGSEPATSGSETPGTSESATPESG |
| | | PGTSTEPSEGSAPGSPAGSPTSTEEGTSESATPESGPGSEPATSGSETPGTSE |
| | | SATPESGPGSPAGSPTSTEEGSPAGSPTSTEEGTSTEPSEGSAPGTSESATPE |
| | | SGPGTSESATPESGPGftabTSESATPESGPGSEPATSGPTESGSEPATSGSE |
| | | TPGSPAGSPTSTEEGTSTEPSEGSAPGTESTPSEGSAPGSEPATSGSETPGTS |
| | | ESATPESGPGTSTEPSEGSAPGEPEA |
| 890 | C-terminal | PGSPAGSPTSTEEGTSESATPESGPGTSTEPSEGSAPGSPAGSPTSTEEGTST |
| (previously | XTEN | EPSEGSAPGTSTEPSEGSAPGTSESATPESGPGSEPATSGSETPGSEPATSGS |
| 8002) | | ETPGSPAGSPTSTEEGTSESATPESGPGTSTEPSEGSAPGTSTEPSEGSAPGS |
| | | PAGSPTSTEEGTSTEPSEGSAPGTSTEPSEGSAPGTSESATPESGPGTSTEPS |
| | | EGSAPGTSESATPESGPGSEPATSGSETPGTSTEPSEGSAPGTSTEPSEGSAP |
| | | GTSESATPESGPGTSESATPESGPGSPAGSPTSTEEGTSESATPESGPGSEPA |
| | | TSGSETPGTSESATPESGPGTSTEPSEGSAPGTSTEPSEGSAPGTSTEPSEGS |
| | | APGTSTEPSEGSAPGTSTEPSEGSAPGTSTEPSEGSAPGSPAGSPTSTEEGTS |
| | | TEPSEGSAPGTSESATPESGPGSEPATSGSETPGTSESATPESGPGSEPATSG |
| | | SETPGTSESATPESGPGTSTEPSEGSAPGTSESATPESGPGSPAGSPTSTEEG |
| | | SPAGSPTSTEEGSPAGSPTSTEEGTSESATPESGPGTSTEPSEGSAPGTSESA |
| | | TPESGPGSEPATSGSETPGTSESATPESGPGSEPATSGSETPGTSESATPESG |
| | | PGTSTEPSEGSAPGSPAGSPTSTEEGTSESATPESGPGSEPATSGSETPGTSE |
| | | SATPESGPGSPAGSPTSTEEGSPAGSPTSTEEGTSTEPSEGSAPGTSESATPE |
| | | SGPGTSESATPESGPGTSESATPESGPGSEPATSGPTESGSEPATSGSETPGS |
| | | PAGSPTSTEEGTSTEPSEGSAPGTSTEPSEGSAPGSEPATSGSETPGTSESAT |
| | | PESGPGTSTEPSEGSAPGEPEA |
| 891 | C-terminal | PGSPAGSPTSTEEGTSESATPESGPGTSTEPSEGSAPGSPAGSPTSTEEGTST |
| (previously | XTEN | EPSEGSAPGTSTEPSEGSAPGTSESATPESGPGSEPATSGSETPGSEPATSGS |
| 8003) | | ETPGSPAGSPTSTEEGTSESATPESGPGTSTEPSEGSAPGTSTEPSEGSAPGS |
| | | PAGSPTSTEEGTSTEPSEGSAPGTSTEPSEGSAPGTSESATPESGPGTSTEPS |
| | | EGSAPGTSESATPESGPGSEPATSGSETPGTSTEPSEGSAPGTSTEPSEGSAP |
| | | GTSESATPESGPGTSESATPESGPGSPAGSPTSTEEGTSESATPESGPGSEPA |
| | | TSGSETPGTSESATPESGPGTSTEPSEGSAPGTSTEPSEGSAPGTSTEPSEGS |
| | | APGTSTEPSEGSAPGTSTEPSEGSAPGTSTEPSEGSAPGSPAGSPTSTEEGTS |
| | | TEPSEGSAPGTSESATPESGPGSEPATSGSETPGTSESATPESGPGSEPATSG |
| | | SETPGTSESATPESGPGTSTEPSEGSAPGTSESATPESGPGSPAGSPTSTEEG |
| | | SPAGSPTSTEEGSPAGSPTSTEEGTSESATPESGPGTSTEPSEGSAPGTSESA |
| | | TPESGPGSEPATSGSETPGTSESATPESGPGSEPATSGSETPGTSESATPESG |
| | | PGTSTEPSEGSAPGSPAGSPTSTEEGTSESATPESGPGSEPATSGSETPGTSE |
| | | SATPESGPGSPAGSPTSTEEGSPAGSPTSTEEGTSTEPSEGSAPGTSESATPE |
| | | SGPGTSESATPESGPGTSESATPESGPGSEPATSGSETPGSEPATSGSETPGS |
| | | PAGSPTSTEEGTSTEPSEGSAPGTESTPSEGSAPGSEPATSGSETPGTSESAT |
| | | PESGPGTSTEPSEGSAPGEPEA |
| 892 | N-terminal | ASSPAGSPTSTESGTSESATPESGPGTETEPSEGSAPGTSESATPESGPGSEP |
| (previously | XTEN | ATSGSETPGTSESATPESGPGSTPAESGSETPGTSESATPESGPGTSTEPSEG |
| 8004) | | SAPGSPAGSPTSTEEGTSESATPESGPGESPATSGSTPEGTSESATPESGPGS |
| | | PAGSPTSTEEGSPAGSPTSTEEGTSTEPSEGSAPGTSESATPESGPGTSESAT |
| | | PESGPGTSESATPESGPGSEPATSGSETPGSEPATSGSETPGSPAGSPTSTEE |
| | | GTSTEPSEGSAPGTSTEPSEGSAPGGSAP |
| 893 | N-terminal | ASSPAGSPTSTESGTSESATPESGPGTSTEPSEGSAPGTSESATPESGPGSEP |
| (previously | XTEN | ATSGSETPGTSESATPESGPGSEPATSGSETPGTSESATPESGPGTSTEPSEG |
| 8005) | | SAPGSPAGSPTSTEEGTSESATPESGPGESPATSGSTPEGTSESATPESGPGS |
| | | PAGSPTSTEEGSPAGSPTSTEEGTSTEPSEGSAPGTSESATPESGPGTSESAT |
| | | PESGPGTSESATPESGPGSEPATSGSETPGSEPATSGSETPGSPAGSPTSTEE |
| | | GTSTEPSEGSAPGTSTEPSEGSAPGGSAP |
| 894 | N-terminal | ASSPAGSPTSTESGTSESATPESGPGTSTEPSEGSAPGTSESATPESGPGSEP |
| (previously | XTEN | ATSGSETPGTSESATPESGPGSTPAESGSETPGTSESATPESGPGTSTEPSEG |
| 8006) | | SAPGSPAGSPTSTEEGTSESATPESGPGEEPATSGSTPEGTSESATPESGPGS |
| | | PAGSPTSTEEGSPAGSPTSTEEGTSTEPSEGSAPGTSESATPESGPGTSESAT |
| | | PESGPGTSESATPESGPGSEPATSGSETPGSEPATSGSETPGSPAGSPTSTEE |
| | | GTSTEPSEGSAPGTSTEPSEGSAPGGSAP |
| 895 | N-terminal | ASSPAGSPTSTESGTSESATPESGPGTSTEPSEGSAPGTSESATPESGPGSEP |
| (previously | XTEN | ATSGSETPGTSESATPESGPGSTPAESGSETPGTSESATPESGPGTSTEPSEG |
| 8007) | | SAPGSPAGSPTSTEEGTSESATPESGPGSEPATSGSETPGTSESATPESGPGS |
| | | PAGSPTSTEEGSPAGSPTSTEEGTSTEPSEGSAPGTSESATPESGPGTSESAT |
| | | PESGPGTSESATPESGPGSEPATSGSETPGSEPATSGSETPGSPAGSPTSTEE |
| | | GTSTEPSEGSAPGTSTEPSEGSAPGGSAP |
| 896 | C-terminal | PGSPAGSPTSTEEGTSESATPESGPGTSTEPSEGSAPGSPAGSPTSTEEGTST |
| (previously | XTEN | EPSEGSAPGTSTEPSEGSAPGTSESATPESGPGSEPATSGSETPGSEPATSGS |
| 8008) | | ETPGSPAGSPTSTEEGTSESATPESGPGTSTEPSEGSAPGTSTEPSEGSAPGS |
| | | PAGSPTSTEEGTSTEPSEGSAPGTSTEPSEGSAPGTSESATPESGPGTSTEPS |
| | | EGSAPGTSESATPESGPGSEPATSGSETPGTSTEPSEGSAPGTSTEPSEGSAP |
| | | GTSESATPESGPGTSESATPESGPGSPAGSPTSTEEGTSESATPESGPGSEPA |
| | | TSGSETPGTSESATPESGPGTSTEPSEGSAPGTSTEPSEGSAPGTSTEPSEGS |
| | | APGTSTEPSEGSAPGTSTEPSEGSAPGTSTEPSEGSAPGSPAGSPTSTEEGTS |
| | | TEPSEGSAPGTSESATPESGPGSEPATSGSETPGTSESATPESGPGSEPATSG |
| | | SETPGTSESATPESGPGTSTEPSEGSAPGTSESATPESGPGSPAGSPTSTEEG |
| | | SPAGSPTSTEEGSPAGSPTSTEEGTSESATPESGPGTSTEPSEGSAPGTSESA |
| | | TPESGPGSEPATSGSETPGTSESATPESGPGSEPATSGSETPGTSESATPESG |
| | | PGTSTEPSEGSAPGSPAGSPTSTEEGTSESATPESGPGSEPATSGSETPGTSE |
| | | SATPESGPGSPAGSPTSTEEGSPAGSPTSTEEGTSTEPSEGSAPGTSESATPE |
| | | SGPGTSESATPESGPGTSESATPESGPGSEPATSGSETPGSEPATSGSETPGS |
| | | PAGSPTSTEEGTSTEPSEGSAPGTESTPSEGSAPGSEPATSGSETPGTSESAT |
| | | PESGPGTSTEPSEGSAPG |
| 897 | C-terminal | PGSPAGSPTSTEEGTSESATPESGPGSEPATSGSETPGTSESATPESGPGTST |
| (previously | XTEN | EPSEGSAPGTSTEPSEGSAPGTSTEPSEGSAPGTSTEPSEGSAPGTSTEPSEG |
| 8009) | | SAPGTSTEPSEGSAPGSPAGSPTSTEEGTSTEPSEGSAPGTSESATPESGPGS |
| | | EPATSGSETPGTSESATPESGPGSEPATSGSETPGTSESATPESGPGTSTEPS |
| | | EGSAPGTSESATPESGPGSPAGSPTSTEEGSPAGSPTSTEEGSPAGSPTSTEE |
| | | GTSESATPESGPGTSTEPSEGSAPGTSESATPESGPGSEPATSGSETPGTSES |
| | | ATPESGPGSEPATSGSETPGTSESATPESGPGTSTEPSEGSAPGSPAGSPTST |
| | | EEGTSESATPESGPGSEPATSGSETPGTSESATPESGPGSPAGSPTSTEEGSP |
| | | AGSPTSTEEGTSTEPSEGSAPGTSESATPESGPGTSESATPESGPGTSESATP |
| | | ESGPGSEPATSGSETPGSEPATSGSETPGSPAGSPTSTEEGTSTEPSEGSAPG |
| | | TESTPSEGSAPGSEPATSGSETPGTSESATPESGPGTSTEPSEGSAPG |
| 898 | N-terminal | SAGSPGSPAGSPTSTEEGTSESATPESGPGTSTEPSEGSAPGSPAGSPTSTEE |
| (previously | XTEN | GTSTEPSEGSAPGTSTEPSEGSAPGTSESATPESGPGSTPAESGSETPGSEPA |
| 8010) | | TSGSETPGSPAGSPTSTEEGTSESATPESGPGTSTEPSEGSAPGTSTEPSEGS |
| | | APGSPAGSPTSTEEGTSTEPSEGSAPGTSTEPSEGSAPGTSESATPESGPGTS |
| | | TEPSEGSAPGTSESATPESGPGSEPATSGSTETPGTSTEPSEGSAPGTSTEPS |
| | | EGSAPGTSESATPESGPGTSESATPESGPGSPAGSPTSTEEGTSESATPESGP |
| | | GSEPATSGSETPGTSESATPESGPGTSTEPSEGSAPGTSTEPSEGSAPGTSTE |
| | | PSEGSAPGTSTEPSEGSAPGTSTEPSEGSAPGTSTEPSEGSAPGSPAGSPTST |
| | | EEGTSTEPSEGSAPGTSESATPESGPGSEPATSGSETPGTSESATPESGPGSE |
| | | PATSGSETPGTSESATPESGPGTSTEPSEGSAPGTSESATPESGPGSPAGSPT |
| | | STEEGSPAGSPTSTEEGSPAGSPTSTEEGTSESATPESGPGTSTEPSEGSAPG |
| | | TSESATPESGPGSEPATSGSETPGTSESATPESGPGSEPATSGSETPGTSESA |
| | | TPESGPGTSTEPSEGSAPGSPAGSPTSTEEGTSESATPESGPGSEPATSGSET |
| | | PGTSESATPESGPGSPAGSPTSTEEGSPAGSPTSTEEGTSTEPSEGSAPGTSE |
| | | SATPESGPGTSESATPESGPGTSESATPESGPGSEPATSGSETPGSEPATSGS |
| | | ETPGSPAGSPTSTEEGTSTEPSEGSAPGTSTEPSEGSAPGSEPATSGSETPGT |
| | | SESATPESGPGTSTEPSEGSAPGSPAGSPTSTEEGTSESATPESGPGTSTEPS |
| | | EGSAPGSPAGSPTSTEEGTSTEPSEGSAPGTSTEPSEGSAPGTSESATPESGP |
| | | GSEPATSGSETPGSEPATSGSETPGSPAGSPTSTEEGTSESATPESGPGTSTE |
| | | PSEGSAPGTSTEPSEGSAPGSPAGSPTSTEEGTSTEPSEGSAPGTSTEPSEGS |
| | | APGTSESATPESGPGTSTEPSEGSAPGTSESATPESGPGSEPATSGSETPGTS |
| | | TEPSEGSAPGTSTEPSEGSAPGTSESATPESGPGTESAS |
| 899 | C-terminal | SAGSPGSPAGSPTSTEEGTSESATPESGPGTSTEPSEGSAPGSPAGSPTSTEE |
| (previously | XTEN | GTSTEPSEGSAPGTSTEPSEGSAPGTSESATPESGPGSEPATSGSETPGSEPA |
| 8011) | | TSGSETPGSPAGSPTSTEEGTSESATPESGPGTSTEPSEGSAPGTSTEPSEGS |
| | | APGSPAGSPTSTEEGTSTEPSEGSAPGTSTEPSEGSAPGTSESATPESGPGTS |
| | | TEPSEGSAPGTSESATPESGPGSEPATSGSETPGTSTEPSEGSAPGTSTEPSE |
| | | GSAPGTSESATPESGPGTSESATPESGPGSPAGSPTSTEEGTSESATPESGPG |
| | | SEPATSGSETPGTSESATPESGPGTSTEPSEGSAPGTSTEPSEGSAPGTSTEP |
| | | SEGSAPGTSTEPSEGSAPGTSTEPSEGSAPGTSTEPSEGSAPGSPAGSPTSTE |
| | | EGTSTEPSEGSAPGTSESATPESGPGSEPATSGSETPGTSESATPESGPGSEP |
| | | ATSGSETPGTSESATPESGPGTSTEPSEGSAPGTSESATPESGPGSPAGSPTS |
| | | TEEGSPAGSPTSTEEGSPAGSPTSTEEGTSESATPESGPGTSTEPSEGSAPGT |
| | | SESATPESGPGSEPATSGSETPGTSESATPESGPGSEPATSGSETPGTSESAT |
| | | PESGPGTSTEPSEGSAPGSPAGSPTSTEEGTSESATPESGPGSEPATSGSETP |
| | | GTSESATPESGPGSPAGSPTSTEEGSPAGSPTSTEEGTSTEPSEGSAPGTSES |
| | | ATPESGPGTSESATPESGPGTSESATPESGPGSEPATSGSETPGSEPATSGSE |
| | | TPGSPAGSPTSTEEGTSTEPSEGSAPGTSTEPSEGSAPGSEPATSGSETPGTS |
| | | ESATPESGPGTSTEPSEGSAPGSPAGSPTSTEEGTSESATPESGPGTSTEPSE |
| | | GSAPGSPAGSPTSTEEGTSTEPSEGSAPGTSTEPSEGSAPGTSESATPESGPG |
| | | SEPATSGSETPGSEPATSGSTETPGSPAGSPTSTEEGTSESATPESGPGTSTE |
| | | PSEGSAPGTSTEPSEGSAPGSPAGSPTSTEEGTSTEPSEGSAPGTATESPEGS |
| | | APGTSESATPESGPGTSTEPSEGSAPGTSAESATPESGPGSEPATSGSETPGT |
| | | STEPSEGSAPGTSTEPSEGSAPGTSESATPESGPGTESAS |
| 900 | N-terminal | GSPAGSPTSTEEGTSESATPESGPGTSTEPSEGSAPGSPAGSPTSTEEGTSTE |
| (previously | XTEN | PSEGSAPGTSTEPSEGSAPATSESATPESGPGSEPATSGSETPGSEPATSGSE |
| 8012) | | TPGSPAGSPTSTEEGTSESASPESGPGTSTEPSEGSAPGTSTEPSEGSAPGSP |
| | | AGSPTSTEEGTSTEPSEGSAPGTSTEPSEGSAPGTSESATPESGPGTSTEPSE |
| | | GSAPGTSESATPESGPGSEPATSGSETPGTSTEPSEGSAPGTSTEPSEGSAPG |
| | | TSESATPESGPGTSESATPESGPGSPAGSPTSTEEGTSESATPESGPGSEPAT |
| | | SGSETPGTSESATPESGPGTSTEPSEGSAPGTSTEPSEGSAPGTSTEPSEGSA |
| | | PGTSTEPSEGSAPGTSTEPSEGSAPGTSTEPSEGSAPGSPAGSPTSTEEGTST |
| | | EPSEGSAPGTSESATPESGPGSEPATSGSETPGTSESATPESGPGSEPATSGS |
| | | ETPGTSESATPESGPGTSTEPSEGSAPGTSESATPESGPGSPAGSPTSTEEGS |
| | | PAGSPTSTEEGSPAGSPTSTEEGTSESATPESGPGTSTEPSEGSAPGTSESAT |
| | | PESGPGSEPATSGSETPGTSESATPESGPGSEPATSGSETPGTSESATPESGP |
| | | GTSTEPSEGSAPGSPAGSPTSTEEGTSESATPESGPGSEPATSGSETPGTSES |
| | | ATPESGPGSPAGSPTSTEEGSPAGSPTSTEEGTSTEPSEGSAPGTSESATPES |
| | | GPGTSESATPESGPGTSESATPESGPGSEPATSGSETPGSEPATSGSETPGSP |
| | | AGSPTSTEEGTSTEPSEGSAPGTSTEPSEGSAPGSEPATSGSETPGTSESATP |
| | | ESGPGTSTEPSEGSAP |
| 901 | N-terminal | GSPAGSPTSTEEGTSESATPESGPGTSTEPSEGSAPGSPAGSPTSTEEGTSTE |
| (previously | XTEN | PSEGSAPGTSTEPSEGSAPGTSESATPESGPGSESATSGSETPGSEPATSGSE |
| 8013) | | TPGSPAGSPTSTEEGTSESATPESGPGTSTEPSEGSAPGTSTEPSEGSAPGSP |
| | | AGSPTSTEEGTSTEPSEGSAPGTSTEPSEGSAPGTSESATPESGPGTSTEPSE |
| | | GSAPGTSESATPESGPGSEPATSGSETPGTSTEPSEGSAPGTSTEPSEGSAPG |
| | | TSESATPESGPGTSESATPESGPGSPAGSPTSTEEGTSESATPESGPGSEPAT |
| | | SGSETPGTSESATPESGPGTSTEPSEGSAPGTSTEPSEGSAPGTSTEPSEGSA |
| | | PGTSTEPSEGSAPGTSTEPSEGSAPGTSTEPSEGSAPGSPAGSPTSTEEGTST |
| | | EPSEGSAPGTSESATPESGPGSEPATSGSETPGTSESATPESGPGSEPATSGS |
| | | ETPGTSESATPESGPGTSTEPSEGSAPGTSESATPESGPGSPAGSPTSTEEGS |
| | | PAGSPTSTEEGSPAGSPTSTEEGTSESATPESGPGTSTEPSEGSAPGTSESAT |
| | | PESGPGSEPATSGSETPGTSESATPESGPGSEPATSGSETPGTSESATPESGP |
| | | GTSTEPSEGSAPGSPAGSPTSTEEGTSESATPESGPGSEPATSGSETPGTSES |
| | | ATPESGPGSPAGSPTSTEEGSPAGSPTSTEEGTSTEPSEGSAPGTSESATPES |
| | | GPGTSESATPESGPGTSESATPESGPGSEPATSGSETPGSEPATSGSETPGSP |
| | | AGSPTSTEEGTSTEPSEGSAPGTSTEPSEGSAPGSEPATSGSETPGTSESATP |
| | | ESGPGTSTEPSEGSAP |
| 902 | N-terminal | SPAGSPTSTESGTSESATPESGPGTSTEPSEGSAPGTSESATPESGPGSEPAT |
| (previously | XTEN (with | SGSETPGTSESATPESGPGSTPAESGSETPGTSESATPESGPGTSTEPSEGSA |
| 8014) | His-tag) | PGSPAGSPTSTEEGTSESATPESGPGSEPATSGSETPGTSESATPESGPGSPA |
| | | GSPTSTEEGSPAGSPTSTEEGTSTEPSEGSAPGTSESATPESGPGTSESATPE |
| | | SGPGTSESATPESGPGSEPATSGSETPGSEPATSGSETPGSPAGSPTSTEEGT |
| | | STEPSEGSAPGTSTEPSEGSAPGGSAP |
| 903 | C-terminal | PGSPAGSPTSTEEGTSESATPESGPGSEPATSGSETPGTSESATPESGPGTST |
| (previously | XTEN | EPSEGSAPGTSTEPSEGSAPGTSTEPSEGSAPGTSTEPSEGSAPGTSTEPSEG |
| 8015) | | SAPGTSTEPSEGSAPGSPAGSPTSTEEGTSTEPSEGSAPGTSESATPESGPGS |
| | | EPATSGSETPGTSESATPESGPGSEPATSGSETPGTSESATPESGPGTSTEPS |
| | | EGSAPGTSESATPESGPGSPAGSPTSTEEGSPAGSPTSTEEGSPAGSPTSTEE |
| | | GTSESATPESGPGTSTEPSEGSAPGTSESATPESGPGSEPATSGSETPGTSES |
| | | ATPESGPGSEPATSGSETPGTSESATPESGPGTSTEPSEGSAPGSPAGSPTST |
| | | EEGTSESATPESGPGSEPATSGSETPGTSESATPESGPGSPAGSPTSTEEGSP |
| | | AGSPTSTEEGTSTEPSEGSAPGTSESATPESGPGTSESATPESGPGTSESATP |
| | | ESGPGSEPATSGSETPGSEPATSGSETPGSPAGSPTSTEEGTSTEPSEGSAPG |
| | | TESTPSEGSAPGSEPATSGSETPGTSESATPESGPGTSTEPSEGSAPGEPEA |
| 904 | C-terminal | TPESGPGTSESATPESGPGSPAGSPTSTEEGTSESATPESGPGSEPATSGSET |
| (previously | XTEN | PGTSESATPESGPGTSTEPSEGSAPGTSTEPSEGSAPGTSTEPSEGSAPGTST |
| 8016) | | EPSEGSAPGTSTEPSEGSAPGTSTEPSEGSAPGSPAGSPTSTEEGTSTEPSEG |
| | | SAPGTSESATPESGPGSEPATSGSETPGTSESATPESGPGSEPATSGSETPGT |
| | | SESATPESGPGTSTEPSEGSAPGTSESATPESGPGSPAGSPTSTEEGSPAGSP |
| | | TSTEEGSPAGSPTSTEEGTSESATPESGPGTSTEPSEGSAPGTSESATPESGP |
| | | GSEPATSGSETPGTSESATPESGPGSEPATSGSETPGTSESATPESGPGTSTE |
| | | PSEGSAPGSPAGSPTSTEEGTSESATPESGPGSEPATSGSETPGTSESATPES |
| | | GPGSPAGSPTSTEEGSPAGSPTSTEEGTSTEPSEGSAPGTSESATPESGPGTS |
| | | ESATPESGPGTSESATPESGPGSEPATSGSETPGSESATSGSETPGSPAGSPT |
| | | STEEGTSTEPSEGSAPGTSTEPSEGSAPGSEPATSGSETPGTSESA |
| 905 | C-terminal | GTSTEPSEGSAPGTSESATPESGPGSEPATSGSETPGSEPATSGSETPGSPAG |
| (previously | XTEN | SPTSTEEGTSESATPESGPGTSTEPSEGSAPGTSTEPSEGSAPGSPAGSPTST |
| 8017) | | EEGTSTEPSEGSAPGTSTEPSEGSAPGTSESATPESGPGTSTEPSEGSAPGTS |
| | | ESATPESGPGSEPATSGSETPGTSTEPSEGSAPGTSTEPSEGSAPGTSESATP |
| | | ESGPGTSESATPESGPGSPAGSPTSTEEGTSESATPESGPGSEPATSGSETPG |
| | | TSESATPESGPGTSTEPSEGSAPGTSTEPSEGSAPGTSTEPSEGSAPGTSTEP |
| | | SEGSAPGTSTEPSEGSAPGTSTEPSEGSAPGSPAGSPTSTEEGTSTEPSEGSA |
| | | PGTSESATPESGPGSEPATSGSETPGTSESATPESGPGSEPATSGSETPGTSE |
| | | SATPESGPGTSTEPSEGSAPGTSESASPESGPGSPAGSPTSTEEGSPAGSPTS |
| | | TEEGSPAGSPTSTEEGTSESATPESGPGTSTEPSEGSAPGTSESATPESGPGS |
| | | EPATSGSETPGTSESATPESGPGSEPATSGSETPGTSESATPESGP |
| 906 | C-terminal | GSETPGSPAGSPTSTEEGTSESATPESGPGTSTEPSEGSAPGTSTEPSEGSAP |
| (previously | XTEN | GSPAGSPTSTEEGTSTEPSEGSAPGTSTEPSEGSAPGTSESATPESGPGTSTE |
| 8018) | | PSEGSAPGTSESATPESGPGSEPATSGSETPGTSTEPSEGSAPGTSTEPSEGS |
| | | APGTSESATPESGPGTSESATPESGPGSPAGSPTSTEEGTSESATPESGPGSE |
| | | PATSGSETPGTSESATPESGPGTSTEPSEGSAPGTSTEPSEGSAPGTSTEPSE |
| | | GSAPGTSTEPSEGSAPGTSTEPSEGSAPGTSTEPSEGSAPGSPAGSPTSTEEG |
| | | TSTEPSEGSAPGTSESATPESGPGSEPATSGSETPGTSESATPESGPGSEPAT |
| | | SGSETPGTSESATPESGPGTSTEPSEGSAPGTSESATPESGPGSPAGSPTSTE |
| | | EGSPAGSPTSTEEGSPAGSPTSTEEGTSESATPESGPGTSTEPSEGSAPGTSE |
| | | SATPESGPGSEPATSGSTETGTSESATPESGPGSEPATSGSETPGTSESATPE |
| | | SGPGTSTEPSEGSAPGSPAGSPTSTEEGTSESATPESGPGSEPATS |
| 907 | C-terminal | EGSAPGTSTEPSEGSAPGTSESATPESGPGTSTEPSEGSAPGTSESATPESGP |
| (previously | XTEN | GSEPATSGSETPGTSTEPSEGSAPGTSTEPSEGSAPGTSESATPESGPGTSES |
| 8019) | | ATPESGPGSPAGSPTSTEEGTSESATPESGPGSEPATSGSETPGTSESATPES |
| | | GPGTSTEPSEGSAPGTSTEPSEGSAPGTSTEPSEGSAPGTSTEPSEGSAPGTS |
| | | TEPSEGSAPGTSTEPSEGSAPGSPAGSPTSTEEGTSTEPSEGSAPGTSESATP |
| | | ESGPGSEPATSGSETPGTSESATPESGPGSEPATSGSETPGTSESATPESGPG |
| | | TSTEPSEGSAPGTSESATPESGPGSPAGSPTSTEEGSPAGSPTSTEEGSPAGS |
| | | PTSTEEGTSESATPESGPGTSTEPSEGSAPGTSESATPESGPGSEPATSGSET |
| | | PGTSESATPESGPGSEPATSGSETPGTSESASPESGPGTSTEPSEGSAPGSPA |
| | | GSPTSTEEGTSESATPESGPGSEPATSGSETPGTSESATPESGPGSPAGSPTS |
| | | TEEGSPAGSPTSTEEGTSTEPSEGSAPGTSESATPESGPGTSESAT |
| 908 | N-terminal | ASSPAGSPTSTESGTSESATPESGPGTSTEPSEGSAPGTSESATPESGPGSEP |
| (previously | | ATSGSETPGTSESATPESGPGSTPAESGSETPGTSESATPESGPGTSTEPSEG |
| 8020) | | SAPGSPAGSPTSTEEGTSESATPESGPGSEPATSGSETPGTSESATPESGPGS |
| | | PAGSPTSTEEGSPAGSPTSTEEGTSTEPSEGSAPGTSESATPESGPGTSESAT |
| | | PESGPGTSESATPESGPGSEPATSGSETPGSEPATSGSETPGSPAGSPTSTEE |
| | | GTSTEPSEGSAPGTSTEPSEGSAPGGSAP |

### Net Charge

In other cases, the XTEN polypeptides can have an unstructured characteristic imparted by incorporation of amino acid residues with a net charge and/or reducing the proportion of hydrophobic amino acids in the XTEN sequence. The overall net charge and net charge density may be controlled by modifying the content of charged amino acids in the XTEN sequences. In some cases, the net charge density of the XTEN of the compositions may be above +0.1 or below −0.1 charges/residue. In other cases, the net charge of a XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more.

Since most tissues and surfaces in a human or animal have a net negative charge, the XTEN sequences can be designed to have a net negative charge to minimize non-specific interactions between the XTEN containing compositions and various surfaces such as blood vessels, healthy tissues, or various receptors. Not to be bound by a particular theory, the XTEN can adopt open conformations due to electrostatic repulsion between individual amino acids of the XTEN polypeptide that individually carry a high net negative charge and that are distributed across the sequence of the XTEN polypeptide. Such a distribution of net negative charge in the extended sequence lengths of XTEN can lead to an unstructured conformation that, in turn, can result in an effective increase in hydrodynamic radius. Accordingly, in one embodiment the invention provides XTEN in which the XTEN sequences contain about 8, 10, 15, 20, 25, or even about 30% glutamic acid. The XTEN of the compositions of the present invention generally have no or a low content of positively charged amino acids. In some cases the XTEN may have less than about 10% amino acid residues with a positive charge, or less than about 7%, or less than about 5%, or less than about 2% amino acid residues with a positive charge. However, the invention contemplates constructs where a limited number of amino acids with a positive charge, such as lysine, may be incorporated into XTEN to permit conjugation between the epsilon amine of the lysine and a reactive group on a peptide, a linker bridge, or a reactive group on a drug or small molecule to be conjugated to the XTEN backbone. In the foregoing, a fusion proteins can be constructed that comprises XTEN, a biologically active protein, plus a chemotherapeutic agent useful in the treatment of diseases or disorders, wherein the maximum number of molecules of the agent incorporated into the XTEN component is determined by the numbers of lysines or other amino acids with reactive side chains (e.g., cysteine) incorporated into the XTEN.

In some cases, an XTEN sequence may comprise charged residues separated by other residues such as serine or glycine, which may lead to better expression or purification behavior. Based on the net charge, XTENs of the subject compositions may have an isoelectric point (pI) of 1.0, 1.5, 2.0, 2.5, 3.0, 3.5, 4.0, 4.5, 5.0, 5.5, 6.0, or even 6.5. In preferred embodiments, the XTEN will have an isoelectric point between 1.5 and 4.5. In these embodiments, the XTEN incorporated into the BPXTEN fusion protein compositions of the present invention would carry a net negative charge under physiologic conditions that may contribute to the unstructured conformation and reduced binding of the XTEN component to mammalian proteins and tissues.

As hydrophobic amino acids can impart structure to a polypeptide, the invention provides that the content of hydrophobic amino acids in the XTEN will typically be less than 5%, or less than 2%, or less than 10% hydrophobic amino acid content. In one embodiment, the amino acid content of methionine and tryptophan in the XTEN component of a BPXTEN fusion protein is typically less than 5%, or less than 2%, and most preferably less than 10%. In another embodiment, the XTEN will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 10% of the total XTEN sequence.

### Low Immunogenicity

In another aspect, the invention provides compositions in which the XTEN sequences have a low degree of immunogenicity or are substantially non-immunogenic. Several factors can contribute to the low immunogenicity of XTEN, e.g., the non-repetitive sequence, the unstructured conformation, the high degree of solubility, the low degree or lack of self-aggregation, the low degree or lack of proteolytic sites within the sequence, and the low degree or lack of conformational epitopes in the XTEN sequence.

Conformational epitopes are formed by regions of the protein surface that are composed of multiple discontinuous amino acid sequences of the protein antigen. The precise folding of the protein brings these sequences into a well-defined, stable spatial configurations, or epitopes, that can be recognized as “foreign” by the host humoral immune system, resulting in the production of antibodies to the protein or triggering a cell-mediated immune response. In the latter case, the immune response to a protein in an individual is heavily influenced by T-cell epitope recognition that is a function of the peptide binding specificity of that individual's HLA-DR allotype. Engagement of an MHC Class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

The ability of a peptide to bind a given MHC Class II molecule for presentation on the surface of an APC (antigen presenting cell) is dependent on a number of factors; most notably its primary sequence. In one embodiment, a lower degree of immunogenicity may be achieved by designing XTEN sequences that resist antigen processing in antigen presenting cells, and/or choosing sequences that do not bind MHC receptors well. The invention provides BPXTEN fusion proteins with substantially non-repetitive XTEN polypeptides designed to reduce binding with MHC II receptors, as well as avoiding formation of epitopes for T-cell receptor or antibody binding, resulting in a low degree of immunogenicity. Avoidance of immunogenicity is, in part, a direct result of the conformational flexibility of XTEN sequences; e.g., the lack of secondary structure due to the selection and order of amino acid residues. For example, of particular interest are sequences having a low tendency to adapt compactly folded conformations in aqueous solution or under physiologic conditions that could result in conformational epitopes. The administration of fusion proteins comprising XTEN, using conventional therapeutic practices and dosing, would generally not result in the formation of neutralizing antibodies to the XTEN sequence, and may also reduce the immunogenicity of the BP fusion partner in the BPXTEN compositions.

In one embodiment, the XTEN sequences utilized in the subject fusion proteins can be substantially free of epitopes recognized by human T cells. The elimination of such epitopes for the purpose of generating less immunogenic proteins has been disclosed previously; see for example WO 98/52976, WO 02/079232, and WO 00/3317 which are incorporated by reference herein. Assays for human T cell epitopes have been described (Stickler, M., et al. (2003) J Immunol Methods, 281: 95-108). Of particular interest are peptide sequences that can be oligomerized without generating T cell epitopes or non-human sequences. This can be achieved by testing direct repeats of these sequences for the presence of T-cell epitopes and for the occurrence of 6 to 15-mer and, in particular, 9-mer sequences that are not human, and then altering the design of the XTEN sequence to eliminate or disrupt the epitope sequence. In some cases, the XTEN sequences are substantially non-immunogenic by the restriction of the numbers of epitopes of the XTEN predicted to bind MHC receptors. With a reduction in the numbers of epitopes capable of binding to MHC receptors, there is a concomitant reduction in the potential for T cell activation as well as T cell helper function, reduced B cell activation or upregulation and reduced antibody production. The low degree of predicted T-cell epitopes can be determined by epitope prediction algorithms such as, e.g., TEPITOPE (Sturniolo, T., et al. (1999) Nat Biotechnol, 17: 555-61). The TEPITOPE score of a given peptide frame within a protein is the log of the K_{d} (dissociation constant, affinity, off-rate) of the binding of that peptide frame to multiple of the most common human MHC alleles, as disclosed in Sturniolo, T. et al. (1999) Nature Biotechnology 17:555). The score ranges over at least 20 logs, from about 10 to about −10 (corresponding to binding constraints of 10ₑ¹⁰ K_{d} to 10e⁻¹⁰ K_{d}), and can be reduced by avoiding hydrophobic amino acids that can serve as anchor residues during peptide display on MHC, such as M, I, L, V, F. In some embodiments, an XTEN component incorporated into a BPXTEN does not have a predicted T-cell epitope at a TEPITOPE score of about −5 or greater, or −6 or greater, or −7 or greater, or −8 or greater, or at a TEPITOPE score of −9 or greater. As used herein, a score of “−9 or greater” would encompass TEPITOPE scores of 10 to −9, inclusive, but would not encompass a score of −10, as −10 is less than −9.

In another embodiment, the inventive XTEN sequences, including those incorporated into the subject BPXTEN fusion proteins, can be rendered substantially non-immunogenic by the restriction of known proteolytic sites from the sequence of the XTEN, reducing the processing of XTEN into small peptides that can bind to MHC II receptors. In another embodiment, the XTEN sequence can be rendered substantially non-immunogenic by the use a sequence that is substantially devoid of secondary structure, conferring resistance to many proteases due to the high entropy of the structure. Accordingly, the reduced TEPITOPE score and elimination of known proteolytic sites from the XTEN may render the XTEN compositions, including the XTEN of the BPXTEN fusion protein compositions, substantially unable to be bound by mammalian receptors, including those of the immune system. In one embodiment, an XTEN of a BPXTEN fusion protein can have >100 nM K_{d} binding to a mammalian receptor, or greater than 500 nM K_{d}, or greater than 1 μM K_{d} towards a mammalian cell surface or circulating polypeptide receptor.

Additionally, the non-repetitive sequence and corresponding lack of epitopes of XTEN can limit the ability of B cells to bind to or be activated by XTEN. A repetitive sequence is recognized and can form multivalent contacts with even a few B cells and, as a consequence of the cross-linking of multiple T-cell independent receptors, can stimulate B cell proliferation and antibody production. In contrast, while a XTEN can make contacts with many different B cells over its extended sequence, each individual B cell may only make one or a small number of contacts with an individual XTEN due to the lack of repetitiveness of the sequence. As a result, XTENs typically may have a much lower tendency to stimulate proliferation of B cells and thus an immune response. In one embodiment, the BPXTEN may have reduced immunogenicity as compared to the corresponding BP that is not fused. In one embodiment, the administration of up to three parenteral doses of a BPXTEN to a mammal may result in detectable anti-BPXTEN IgG at a serum dilution of 1:100 but not at a dilution of 1:1000. In another embodiment, the administration of up to three parenteral doses of a BPXTEN to a mammal may result in detectable anti-BP IgG at a serum dilution of 1:100 but not at a dilution of 1:1000. In another embodiment, the administration of up to three parenteral doses of a BPXTEN to a mammal may result in detectable anti-XTEN IgG at a serum dilution of 1:100 but not at a dilution of 1:1000. In the foregoing embodiments, the mammal can be a mouse, a rat, a rabbit, or a cynomolgus monkey.

An additional feature of XTENs with non-repetitive sequences relative to sequences with a high degree of repetitiveness can be that non-repetitive XTENs form weaker contacts with antibodies. Antibodies are multivalent molecules. For instance, IgGs have two identical binding sites and IgMs contain 10 identical binding sites. Thus antibodies against repetitive sequences can form multivalent contacts with such repetitive sequences with high avidity, which can affect the potency and/or elimination of such repetitive sequences. In contrast, antibodies against non-repetitive XTENs may yield monovalent interactions, resulting in less likelihood of immune clearance such that the BPXTEN compositions can remain in circulation for an increased period of time.

### Increased Hydrodynamic Radius

In another aspect, the present invention provides XTEN in which the XTEN polypeptides can have a high hydrodynamic radius that confers a corresponding increased Apparent Molecular Weight to the BPXTEN fusion protein incorporating the XTEN. The linking of XTEN to BP sequences can result in BPXTEN compositions that can have increased hydrodynamic radii, increased Apparent Molecular Weight, and increased Apparent Molecular Weight Factor compared to a BP not linked to an XTEN. For example, in therapeutic applications in which prolonged half-life is desired, compositions in which a XTEN with a high hydrodynamic radius is incorporated into a fusion protein comprising one or more BP can effectively enlarge the hydrodynamic radius of the composition beyond the glomerular pore size of approximately 3-5 nm (corresponding to an apparent molecular weight of about 70 kDA) (Caliceti. 2003. Pharmacokinetic and biodistribution properties of poly(ethylene glycol)-protein conjugates. Adv Drug Deliv Rev 55:1261-1277), resulting in reduced renal clearance of circulating proteins. The hydrodynamic radius of a protein is determined by its molecular weight as well as by its structure, including shape and compactness. Not to be bound by a particular theory, the XTEN can adopt open conformations due to electrostatic repulsion between individual charges of the peptide or the inherent flexibility imparted by the particular amino acids in the sequence that lack potential to confer secondary structure. The open, extended and unstructured conformation of the XTEN polypeptide can have a greater proportional hydrodynamic radius compared to polypeptides of a comparable sequence length and/or molecular weight that have secondary and/or tertiary structure, such as typical globular proteins. Methods for determining the hydrodynamic radius are well known in the art, such as by the use of size exclusion chromatography (SEC), as described in U.S. Pat. Nos. 6,406,632 and 7,294,513. The addition of increasing lengths of XTEN results in proportional increases in the parameters of hydrodynamic radius, Apparent Molecular Weight, and Apparent Molecular Weight Factor, permitting the tailoring of BPXTEN to desired characteristic cut-off Apparent Molecular Weights or hydrodynamic radii. Accordingly, in certain embodiments, the BPXTEN fusion protein can be configured with an XTEN such that the fusion protein can have a hydrodynamic radius of at least about 5 nm, or at least about 8 nm, or at least about 10 nm, or 12 nm, or at least about 15 nm. In the foregoing embodiments, the large hydrodynamic radius conferred by the XTEN in a BPXTEN fusion protein can lead to reduced renal clearance of the resulting fusion protein, leading to a corresponding increase in terminal half-life, an increase in mean residence time, and/or a decrease in renal clearance rate.

In another embodiment, an XTEN of a chosen length and sequence can be selectively incorporated into a BPXTEN to create a fusion protein that will have, under physiologic conditions, an Apparent Molecular Weight of at least about 100 kDa, at least about 150 kDa, or at least about 300 kDa, or at least about 400 kDa, or at least about 500 kDA, or at least about 600 kDa, or at least about 700 kDA, or at least about 800 kDa, or at least about 900 kDa, or at least about 1000 kDa, or at least about 1200 kDa, or at least about 1500 kDa, or at least about 1800 kDa, or at least about 2000 kDa, or at least about 2300 kDa or more. In another embodiment, an XTEN of a chosen length and sequence can be selectively linked to a BP to result in a BPXTEN fusion protein that has, under physiologic conditions, an Apparent Molecular Weight Factor of at least three, alternatively of at least four, alternatively of at least five, alternatively of at least six, alternatively of at least eight, alternatively of at least 10, alternatively of at least 15, or an Apparent Molecular Weight Factor of at least 20 or greater. In another embodiment, the BPXTEN fusion protein has, under physiologic conditions, an Apparent Molecular Weight Factor that is about 4 to about 20, or is about 6 to about 15, or is about 8 to about 12, or is about 9 to about 10 relative to the actual molecular weight of the fusion protein.

### Biologically Active Proteins of the BPXTEN Fusion Protein Compositions

The present invention relates in part to fusion protein compositions comprising biologically active proteins and XTEN and the uses thereof for the treatment of diseases, disorders or conditions of a subject.

In one aspect, the invention provides at least a first biologically active protein (hereinafter “BP”) covalently linked to a fusion protein comprising one or more extended recombinant polypeptides (“XTEN”), resulting in an XTEN fusion protein composition (hereinafter “BPXTEN”). As described more fully below, the fusion proteins can optionally include spacer sequences that can further comprise cleavage sequences to release the BP from the fusion protein when acted on by a protease.

The term “BPXTEN”, as used herein, is meant to encompass fusion polypeptides that comprise one or two payload regions each comprising a biologically active protein that mediates one or more biological or therapeutic activities and at least one other region comprising at least one XTEN polypeptide.

The BP of the subject compositions, particularly those disclosed in Tables 6, together with their corresponding nucleic acid and amino acid sequences, are well known in the art and descriptions and sequences are available in public databases such as Chemical Abstracts Services Databases (e.g., the CAS Registry), GenBank, The Universal Protein Resource (UniProt) and subscription provided databases such as GenSeq (e.g., Derwent). Polynucleotide sequences may be a wild type polynucleotide sequence encoding a given BP (e.g., either full length or mature), or in some instances the sequence may be a variant of the wild type polynucleotide sequence (e.g., a polynucleotide which encodes the wild type biologically active protein, wherein the DNA sequence of the polynucleotide has been optimized, for example, for expression in a particular species; or a polynucleotide encoding a variant of the wild type protein, such as a site directed mutant or an allelic variant. It is well within the ability of the skilled artisan to use a wild-type or consensus cDNA sequence or a codon-optimized variant of a BP to create BPXTEN constructs contemplated by the invention using methods known in the art and/or in conjunction with the guidance and methods provided herein, and described more fully in the Examples.

The BP for inclusion in the BPXTEN of the invention can include any protein of biologic, therapeutic, prophylactic, or diagnostic interest or function, or that is useful for mediating a biological activity or preventing or ameliorating a disease, disorder or conditions when administered to a subject. Of particular interest are BP for which an increase in a pharmacokinetic parameter, increased solubility, increased stability, or some other enhanced pharmaceutical property is sought, or those BP for which increasing the terminal half-life would improve efficacy, safety, or result in reduce dosing frequency and/or improve patient compliance. Thus, the BPXTEN fusion protein compositions are prepared with various objectives in mind, including improving the therapeutic efficacy of the bioactive compound by, for example, increasing the in vivo exposure or the length that the BPXTEN remains within the therapeutic window when administered to a subject, compared to a BP not linked to XTEN.

A BP of the invention can be a native, full-length protein or can be a fragment or a sequence variant of a biologically active protein that retains at least a portion of the biological activity of the native protein.

In one embodiment, the BP incorporated into the subject compositions can be a recombinant polypeptide with a sequence corresponding to a protein found in nature. In another embodiment, the BP can be sequence variants, fragments, homologs, and mimetics of a natural sequence that retain at least a portion of the biological activity of the native BP. In non-limiting examples, a BP can be a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a protein sequence selected from Tables 6. In one embodiment, a BPXTEN fusion protein can comprise a single BP molecule linked to an XTEN (as described more fully below). In another embodiment, the BPXTEN can comprise a first BP and a second molecule of the same BP, resulting in a fusion protein comprising the two BP linked to one or more XTEN (for example, two molecules of IL-Ira, or two molecules of IL-10). Biologically active proteins including those as therapeutics are typically labile molecules exhibiting short shelf-lives, particularly when formulated in aqueous solutions. In addition, many biologically active peptides and proteins have limited solubility, or become aggregated during recombinant productions, requiring complex solubilization and refolding procedures. Various chemical polymers can be attached to such proteins to modify their properties. Of particular interest are hydrophilic polymers that have flexible conformations and are well hydrated in aqueous solutions. A frequently used polymer is polyethylene glycol (PEG). These polymers tend to have large hydrodynamic radii relative to their molecular weight (Kubetzko, S., et al. (2005) Mol Pharmacol, 68: 1439-54), and can result in enhanced pharmacokinetic properties. Depending on the points of attachment, the polymers tend to have limited interactions with the protein that they have been attached to such that the polymer-modified protein retains its relevant functions. However, the chemical conjugation of polymers to proteins requires complex multi-step processes. Typically, the protein component needs to be produced and purified prior to the chemical conjugation step. In addition, the conjugation step can result in the formation of heterogeneous product mixtures that need to be separated, leading to significant product loss. Alternatively, such mixtures can be used as the final pharmaceutical product, but are difficult to standardize. Some examples are currently marketed PEGylated Interferon-alpha products that are used as mixtures (Wang, B. L., et al. (1998) J Submicrosc Cytol Pathol, 30: 503-9; Dhalluin, C., et al. (2005) Bioconjug Chem, 16: 504-17). Such mixtures are difficult to reproducibly manufacture and characterize as they contain isomers with reduced or no therapeutic activity.

In general, BP will exhibit a binding specificity to a given target or another desired biological characteristic when used in vivo or when utilized in an in vitro assay. For example, the BP can be an agonist, a receptor, a ligand, an antagonist, an enzyme, or a hormone. Of particular interest are BP used or known to be useful for a disease or disorder wherein the native BP have a relatively short terminal half-life and for which an enhancement of a pharmacokinetic parameter (which optionally could be released from the fusion protein by cleavage of a spacer sequence) would permit less frequent dosing or an enhanced pharmacologic effect. Also of interest are BP that have a narrow therapeutic window between the minimum effective dose or blood concentration (Cₘᵢₙ) and the maximum tolerated dose or blood concentration (Cₘₐₓ). In such cases, the linking of the BP to a fusion protein comprising a select XTEN sequence(s) can result in an improvement in these properties, making them more useful as therapeutic or preventive agents compared to BP not linked to XTEN.

The BP can be a cytokine. Cytokines encompassed by the inventive compositions can have utility in the treatment in various therapeutic or disease categories, including but not limited to cancer, rheumatoid arthritis, multiple sclerosis, myasthenia gravis, systemic lupus erythematosus, Alzheimer's disease, Schizophrenia, viral infections (e.g., chronic hepatitis C, AIDS), allergic asthma, retinal neurodegenerative processes, metabolic disorder, insulin resistance, and diabetic cardiomyopathy. Cytokines can be especially useful in treating inflammatory conditions and autoimmune conditions.

The BP can be one or more cytokines. The cytokines refer to proteins (e.g., chemokines, interferons, lymphokines, interleukins, and tumor necrosis factors) released by cells which can affect cell behavior. Cytokines can be produced by a broad range of cells, including but not limited to immune cells such as macrophages, B lymphocytes, T lymphocytes, microglia cells, and mast cells, as well as endothelial cells, fibroblasts, and various stromal cells. A given cytokine can be produced by more than one type of cell. Cytokines can be involved in producing systemic or local immunomodulatory effects.

Certain cytokines can function as pro-inflammatory cytokines. Pro-inflammatory cytokines refer to cytokines involved in inducing or amplifying an inflammatory reaction. Pro-inflammatory cytokines can work with various cells of the immune system, such as neutrophils and leukocytes, to generate an immune response. Certain cytokines can function as anti-inflammatory cytokines. Anti-inflammatory cytokines refer to cytokines involved in the reduction of an inflammatory reaction. Anti-inflammatory cytokines, in some cases, can regulate a pro-inflammatory cytokine response. Some cytokines can function as both pro- and anti-inflammatory cytokines.

Examples of cytokines that are regulatable by systems and compositions of the present disclosure include, but are not limited to lymphokines, monokines, and traditional polypeptide hormones except for human growth hormone. Included among the cytokines are parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha, TGF-beta, TGF-beta1, TGF-beta2, and TGF-beta3; insulin-like growth factor-I and -II; erythropoietin (EPO); Flt-3L; stem cell factor (SCF); osteoinductive factors; interferons (IFNs) such as IFN-α, IFN-β, IFN-γ; colony stimulating factors (CSFs) such as macrophage-CSF (M-CSF); granulocyte-macrophage-CSF (GM-CSF); granulocyte-CSF (G-CSF); macrophage stimulating factor (MSP); interleukins (ILs) such as IL-1, IL-1a, IL-1b, IL-1RA, IL-18, IL-2, IL-3, IL-4, IL-5, IL-6, IL-7, IL-8, IL-9, IL-10, IL-11, IL-12, IL-12b, IL-13, IL-14, IL-15, IL-16, IL-17, IL-20; a tumor necrosis factor such as CD154, LT-beta, TNF-alpha, TNF-beta, 4-1BBL, APRIL, CD70, CD153, CD178, GITRL, LIGHT, OX40L, TALL-1, TRAIL, TWEAK, TRANCE; and other polypeptide factors including LIF, oncostatin M (OSM) and kit ligand (KL). Cytokine receptors refer to the receptor proteins which bind cytokines. Cytokine receptors may be both membrane-bound and soluble.

The target polynucleotide can encode for a cytokine. Non-limiting examples of cytokines include 4-1BBL, activin βA, activin βB, activin βC, activin βE, artemin (ARTN), BAFF/BLyS/TNFSF138, BMP10, BMP15, BMP2, BMP3, BMP4, BMP5, BMP6, BMP7, BMP8a, BMP8b, bone morphogenetic protein 1 (BMP1), CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CD153/CD30L/TNFSF8, CD40L/CD154/TNFSF5, CD40LG, CD70, CD70/CD27L/TNFSF7, CLCF1, c-MPL/CD110/TPOR, CNTF, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, EDA-A1, FAM19A1, FAM19A2, FAM19A3, FAM19A4, FAM19A5, Fas Ligand/FASLG/CD95L/CD178, GDF10, GDF11, GDF15, GDF2, GDF3, GDF4, GDF5, GDF6, GDF7, GDF8, GDF9, glial cell line-derived neurotrophic factor (GDNF), growth differentiation factor 1 (GDF1), IFNA1, IFNA10, IFNA13, IFNA14, IFNA2, IFNA4, IFNA5/IFNaG, IFNA7, IFNA8, IFNB1, IFNE, IFNG, IFNZ, IFNω/IFNW1, IL11, IL18, IL18BP, ILIA, IL1B, IL1F10, IL1F3/IL1RA, IL1F5, IL1F6, IL1F7, IL1F8, IL1F9, IL1RL2, IL31, IL33, IL6, IL8/CXCL8, inhibin-A, inhibin-B, Leptin, LIF, LTA/TNFB/TNFSF1, LTB/TNFC, neurturin (NRTN), OSM, OX-40L/TNFSF4/CD252, persephin (PSPN), RANKL/OPGL/TNFSFII(CD254), TL1A/TNFSF15, TNFA, TNF-alpha/TNFA, TNFSF10/TRAIL/APO-2L(CD253), TNFSF12, TNFSF13, TNFSF14/LIGHT/CD258, XCL1, and XCL2. In some embodiments, the target gene encodes for an immune checkpoint inhibitor. Non-limiting examples of such immune checkpoint inhibitors include PD-1, CTLA-4, LAG3, TIM-3, A2AR, B7-H3, B7-H4, BTLA, IDO, KIR, and VISTA. In some embodiments, the target gene encodes for a T cell receptor (TCR) alpha, beta, gamma, and/or delta chain.

In some cases, the cytokine can be a chemokine. The chemokine can be selected from a group including, but not limited to, ARMCX2, BCA-1/CXCL13, CCL11, CCL12/MCP-5, CCL13/MCP-4, CCL15/MIP-5/MIP-1 delta, CCL16/HCC-4/NCC4, CCL17/TARC, CCL18/PARC/MIP-4, CCL19/MIP-3b, CCL2/MCP-1, CCL20/MIP-3 alpha/MIP3A, CCL21/6Ckine, CCL22/MDC, CCL23/MIP3, CCL24/Eotaxin-2/MPIF-2, CCL25/TECK, CCL26/Eotaxin-3, CCL27/CTACK, CCL28, CCL3/Mip1a, CCL4/MIP1B, CCL4L1/LAG-1, CCL5/RANTES, CCL6/C10, CCL8/MCP-2, CCL9, CML5, CXCL1, CXCL10/Crg-2, CXCL12/SDF-1 beta, CXCL14/BRAK, CXCL15/Lungkine, CXCL16/SR-PSOX, CXCL17, CXCL2/MIP-2, CXCL3/GRO gamma, CXCL4/PF4, CXCL5, CXCL6/GCP-2, CXCL9/MIG, FAM19A1, FAM19A2, FAM19A3, FAM19A4/TAFA4, FAM19A5, Fractalkine/CX3CL1, I-309/CCL1/TCA-3, IL-8/CXCL8, MCP-3/CCL7, NAP-2/PPBP/CXCL7, XCL2, and Armo IL10.

Table 3 provides a non-limiting list of such sequences of BPs that are encompassed by the BPXTEN fusion proteins of the invention. Metabolic proteins of the inventive BPXTEN compositions can be a protein that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a protein sequence selected from Table 3.

TABLE 3
Cytokines for Conjugation

| Name of Protein | |
|---|---|
| (Synonym) | Sequence |
| Anti-CD3 | See U.S. Pat. Nos. 5,885,573 and 6,491,916 |
| IL-1ra, human | MEICRGLRSHLITLLLFLFHSETICRPSGRKSSKMQAFRIWDVNQKTFYLRNNQLVAGYLQGPNV |
| full length | NLEEKIDVVPIEPHALFLGIHGGKMCLSCVKSGDETRLQLEAVNITDLSENRKQDKRFAFIRSDS |
| | GPTTSFESAACPGWFLCTAMEADQPVSLTNMPDEGVMVTKFYFQEDE (SEQ ID NO: 152) |
| IL-1ra, Dog | METCRCPLSYLISFLLFLPHSETACRLGKRPCRMQAFRIWDVNQKTFYLRNNQLVAGYLQGSNTK |
| | LEEKLDVVPVEPHAVFLGIHGGKLCLACVKSGDETRLQLEAVNITDLSKNKDQDKRFTFILSDSG |
| | PTTSFESAACPGWFLCTALEADRPVSLTNRPEEAMMVTKFYFQKE (SEQ ID NO: 153) |
| IL-1ra, Rabbit | MRPSRSTRRHLISLLLFLFHSETACRPSGKRPCRMQAFRIWDVNQKTFYLRNNQLVAGYLQGPNA |
| | KLEERIDVVPLEPQLLFLGIQRGKLCLSCVKSGDKMKLHLEAVNITDLGKNKEQDKRFTFIRSNS |
| | GPTTTFESASCPGWFLCTALEADQPVSLTNTPDDSIVVTKFYFQED (SEQ ID NO: 154) |
| IL-1ra, Rat | MEICRGPYSHLISLLLILLFRSESAGHIPAGKRPCKMQAFRIWDTNQKTFYLRNNQLIAGYLQGP |
| | NTKLEEKIDMVPIDFRNVFLGIHGGKLCLSCVKSGDDTKLQLEEVNITDLNKNKEEDKRFTFIRS |
| | ETGPTTSFESLACPGWFLCTTLEADHPVSLINTPKEPCTVTKFYFQED (SEQ ID NO: 155) |
| IL-1ra, Mouse | MEICWGPYSHLISLLLILLFHSEAACRPSGKRPCKMQAFRIWDTNQKTFYLRNNQLIAGYLQGPN |
| | IKLEEKIDMVPIDLHSVFLGIHGGKLCLSCAKSGDDIKLQLEEVNITDLSKNKEEDKRFTFIRSE |
| | KGPTTSFESAACPGWFLCTTLEADRPVSLINTPEEPLIVTKFYFQEDQ (SEQ ID NO: 156) |
| Anakinra | MRPSGRKSSKMQAFRIWDVNQKTFYLRNNQLVAGYLQGPNVNLEEKIDVVPIEPHALFLGIHGGK |
| | MCLSCVKSGDETRLQLEAVNITDLSENRKQDKRFAFIRSDSGPTTSFESAACPGWFLCTAMEADQ |
| | PVSLTNMPDEGVMVTKFYFQEDE (SEQ ID NO: 157) |
| IL-10 | MHSSALLCCLVLLTGVRASPGQGTQSENSCTHFPGNLPNMLRDLRDAFSRVKTFFQMKDQLDNLL |
| | LKESLLEDFKGYLGCQALSEMIQFYLEEVMPQAENQDPDIKAHVNSLGENLKTLRLRLRRCHRFL |
| | PCENKSKAVEQVKNAFNKLQEKGIYKAMSEFDIFINYIEAYMTMKIRN (SEQ ID NO: 158) |

TABLE A
Amino acid sequences of exemplary interleukin-12 (IL-12) or fragments thereof

| | SEQ ID | |
|---|---|---|
| Name | NO. | Amino Acid Sequence |
| Interleukin- | 5 | MWELEKDVYVVEVDWTPDAPGETVNLTCDTPEEDDITWTSDQRHGVIGSGKTLT |
| 12 subunit | | ITVKEFLDAGQYTCHKGGETLSHSHLLLHKKENGIWSTEILKNFKNKTFLKCEA |
| beta (IL-12 | | PNYSGRFTCSWLVQRNMDLKFNIKSSSSSPDSRAVTCGMASLSAEKVTLDQRDY |
| p40) | | EKYSVSCQEDVTCPTAEETLPIELALEARQQNKYENYSTSFFIRDIIKPDPPKN |
| | | LQMKPLKNSQVEVSWEYPDSWSTPHSYFSLKFFVRIQRKKEKMKETEEGCNQKG |
| | | AFLVEKTSTEVQCKGGNVCVQAQDRYYNSSCSKWACVPCRVRS |
| Interleukin- | 6 | RVIPVSGPARCLSQSRNLLKTTDDMVKTAREKLKHYSCTAEDIDHEDITRDQTS |
| 12 subunit | | TLKTCLPLELHKNESCLATRETSSTTRGSCLPPQKTSLMMTLCLGSIYEDLKMY |
| alpha (IL-12 | | QTEFQAINAALQNHNHQQIILDKGMLVAIDELMQSLNHNGETLRQKPPVGEADP |
| p35) | | YRVKMKLCILLHAFSTRVVTINRVMGYLSSA |
| IL-12 variant | 7 | MWELEKDVYVVEVDWTPDAPGETVNLTCDTPEEDDITWTSDQRHGVIGSGKTLT |
| | | ITVKEFLDAGQYTCHKGGETLSHSHLLLHKKENGIWSTEILKNFKNKTFLKCEA |
| | | PNYSGRFTCSWLVQRNMDLKFNIKSSSSSPDSRAVTCGMASLSAEKVTLDQRDY |
| | | EKYSVSCQEDVTCPTAEETLPIELALEARQQNKYENYSTSFFIRDIIKPDPPKN |
| | | LQMKPLKNSQVEVSWEYPDSWSTPHSYFSLKFFVRIQRKKEKMKETEEGCNQKG |
| | | AFLVEKTSTEVQCKGGNVCVQAQDRYYNSSCSKWACVPCRVRSGGGGSGGGGSG |
| | | GGGSRVIPVSGPARCLSQSRNLLKTTDDMVKTAREKLKHYSCTAEDIDHEDITR |
| | | DQTSTLKTCLPLELHKNESCLATRETSSTTRGSCLPPQKTSLMMTLCLGSIYED |
| | | LKMYQTEFQAINAALQNHNHQQIILDKGMLVAIDELMQSLNHNGETLRQKPPVG |
| | | EADPYRVKMKLCILLHAFSTRVVTINRVMGYLSSA |

Table A provides a non-limiting list of interleukin-12 sequences (or fragments thereof). The inventive BPXTEN compositions of this disclosure can contain an amino acid sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a protein sequence selected from Table A.

In some embodiments, where the composition of this disclosure (such as a fusion protein) comprises a cytokine, the cytokine can be selected from a group consisting of interleukins, chemokines, interferons, tumor necrosis factors, colony-stimulating factors, or transforming growth factor beta (TGF-beta) superfamily members. In some embodiments, the cytokine can be an interleukin selected from the group consisting of IL1, IL2, IL3, IL4, IL5, IL6, IL7, IL8, IL9, IL10, IL11, IL12, IL13, IL14, IL15, IL16, and IL17. In some embodiments, the cytokine can have at least (about) 80%, at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence selected from Table 3 or Table A. In some embodiments, the cytokine can have at least (about) 80%, at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence selected from Table 3. In some embodiments, the cytokine can have at least (about) 80%, at least (about) 81%, at least (about) 82%, at least (about) 83%, at least (about) 84%, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence selected from Table A. In some embodiments, the cytokine can be IL-12 or an IL-12 variant. In some embodiments, the cytokine can comprise a first cytokine fragment (Cy1) and a second cytokine fragment (Cy2). In some embodiments, one of the Cy1 and the Cy2 can comprise an amino acid sequence having at least 70% sequence identity to an interleukin-12 subunit beta. In some embodiments, the other one of the Cy1 and the Cy2 can comprise an amino acid sequence having at least (about) 70%, at least (about) 75%, at least (about) 80%, at least (about) 85%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to an interleukin-12 subunit alpha. In some embodiments, the first cytokine fragment (Cy1) can comprise an amino acid sequence having at least (about) 70%, at least (about) 75%, at least (about) 80%, at least (about) 85%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence of SEQ ID NO. 5. In some embodiments, the second cytokine fragment (Cy2) can comprise an amino acid sequence having at least (about) 70%, at least (about) 75%, at least (about) 80%, at least (about) 85%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to a sequence of SEQ ID NO. 6. In some embodiments, the cytokine can comprise a linker positioned between the first cytokine fragment (Cy1) and the second cytokine fragment (Cy2). In some embodiments, the cytokine can be an IL-12 variant comprising an amino acid sequence having at least (about) 70%, at least (about) 75%, at least (about) 80%, at least (about) 85%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or 100% sequence identity to SEQ ID NO. 7. The linker can be a GS linker (such as (GGGGS)₁(SEQ ID NO: 273), (GGGGS)₂(SEQ ID NO: 273), (GGGGS)₃(SEQ ID NO: 273), (GGGGS)₄(SEQ ID NO: 273), (GGGGS)s(SEQ ID NO: 273), etc.).

“IL-Ira” means the human IL-1 receptor antagonist protein and species and sequence variants thereof, including the sequence variant anakinra (Kineret®), having at least a portion of the biological activity of nature IL-1ra. Human IL-1ra is a mature glycoprotein of 152 amino acid residues. The inhibitory action of IL-Ira results from its binding to the type I IL-1 receptor. The protein has a native molecular weight of 25 kDa, and the molecule shows limited sequence homology to IL-1α (19%) and IL-1ß (26%). Anakinra is a nonglycosylated, recombinant human IL-Ira and differs from endogenous human IL-Ira by the addition of an N-terminal methionine. A commercialized version of anakinra is marketed as Kineret®. It binds with the same avidity to IL-1 receptor as native IL-1ra and IL-1b, but does not result in receptor activation (signal transduction), an effect attributed to the presence of only one receptor binding motif on IL-Ira versus two such motifs on IL-1α and IL-1ß. Anakinra has 153 amino acids and 17.3 kD in size, and has a reported half-life of approximately 4-6 hours.

Increased IL-1 production has been reported in patients with various viral, bacterial, fungal, and parasitic infections; intravascular coagulation; high-dose IL-2 therapy; solid tumors; leukemias; Alzheimer's disease; HIV-1 infection; autoimmune disorders; trauma (surgery); hemodialysis; ischemic diseases (myocardial infarction); noninfectious hepatitis; asthma; UV radiation; closed head injury; pancreatitis; peritonitis; graft-versus-host disease; transplant rejection; and in healthy subjects after strenuous exercise. There is an association of increased IL-1b production in patients with Alzheimer's disease and a possible role for IL1 in the release of the amyloid precursor protein. IL-1 has also been associated with diseases such as type 2 diabetes, obesity, hyperglycemia, hyperinsulinemia, type 1 diabetes, insulin resistance, retinal neurodegenerative processes, disease states and conditions characterized by insulin resistance, acute myocardial infarction (AMI), acute coronary syndrome (ACS), atherosclerosis, chronic inflammatory disorders, rheumatoid arthritis, degenerative intervertebral disc disease, sarcoidosis, Crohn's disease, ulcerative colitis, gestational diabetes, excessive appetite, insufficient satiety, metabolic disorders, glucagonomas, secretory disorders of the airway, osteoporosis, central nervous system disease, restenosis, neurodegenerative disease, renal failure, congestive heart failure, nephrotic syndrome, cirrhosis, pulmonary edema, hypertension, disorders wherein the reduction of food intake is desired, irritable bowel syndrome, myocardial infarction, stroke, post-surgical catabolic changes, hibernating myocardium, diabetic cardiomyopathy, insufficient urinary sodium excretion, excessive urinary potassium concentration, conditions or disorders associated with toxic hypervolemia, polycystic ovary syndrome, respiratory distress, chronic skin ulcers, nephropathy, left ventricular systolic dysfunction, gastrointestinal diarrhea, postoperative dumping syndrome, irritable bowel syndrome, critical illness polyneuropathy (CIPN), systemic inflammatory response syndrome (SIRS), dyslipidemia, reperfusion injury following ischemia, and coronary heart disease risk factor (CHDRF) syndrome. IL-1ra-containing fusion proteins of the invention may find particular use in the treatment of any of the foregoing diseases and disorders. IL-1ra has been cloned, as described in U.S. Pat. Nos. 5,075,222 and 6,858,409.

In some cases, the BP can be IL-10. IL-10 can be an effective anti-inflammatory cytokine that represses the production of the proinflammatory cytokines and chemokines. IL-10 is the one of the major TH2-type cytokine that increases humoral immune responses and lowers cell-mediated immune reactions. IL-10 can be useful for the treatment of autoimmune diseases and inflammatory diseases such as rheumatoid arthritis, multiple sclerosis, myasthenia gravis, systemic lupus erythematosus, Alzheimer's disease, Schizophrenia, allergic asthma, retinal neurodegenerative processes, and diabetes.

In some cases, IL-10 can be modified to improve stability and decrease prolytic degradation. The modification can be one or more amide bond substitution. In some cases, one or more amide bonds within backbone of IL-10 can be substituted to achieve the abovementioned effects. The one or more amide linkages (—CO—NH—) in IL-10 can be replaced with a linkage which is an isostere of an amide linkage, such as —CH2NH—, —CH₂S—, —CH2CH2-, —CH CH-(cis and trans), —COCH2-, —CH(OH)CH2— or —CH2SO—. Furthermore, the amide linkages in IL-10 can also be replaced by a reduced isostere pseudopeptide bond. See Couder et al. (1993) Int. J. Peptide Protein Res. 41:181-184, which is hereby incorporated by reference in its entirety.

The one or more acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids; and side chain amide residues such as asparagine, glutamine, and alkyl or aromatic substituted derivatives of asparagine or glutamine; as well as hydroxyl-containing amino acids, including serine, threonine, homoserine, 2,3-diaminopropionic acid, and alkyl or aromatic substituted derivatives of serine or threonine can be substituted.

The one or more hydrophobic amino acids in IL-10 such as alanine, leucine, isoleucine, valine, norleucine, (S)-2-aminobutyric acid, (S)-cyclohexylalanine or other simple alpha-amino acids can be substituted with amino acids including, but not limited to, an aliphatic side chain from C1-C10 carbons including branched, cyclic and straight chain alkyl, alkenyl or alkynyl substitutions

In some cases, the one or more hydrophobic amino acids in IL-10 such as can be substituted substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C1-C4)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

The one or more hydrophobic amino acids in IL-10 such as phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkox can be substituted by aromatic amino acids including: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine

The amino acids comprising basic side chains, including arginine, lysine, histidine, ornithine, 2,3-diaminopropionic acid, homoarginine, including alkyl, alkenyl, or aryl-substituted derivatives of the previous amino acids, can be substituted. Examples are N-epsilon-isopropyl-lysine, 3-(4-tetrahydropyridyl)-glycine, 3-(4-tetrahydropyridyl)-alanine, N,N-gamma,gamma′-diethyl-homoarginine, alpha-methyl-arginine, alpha-methyl-2,3-diaminopropionic acid, alpha-methyl-histidine, and alpha-methyl-ornithine where the alkyl group occupies the pro-R position of the alpha-carbon. The modified IL-10 can comprise amides formed from any combination of alkyl, aromatic, heteroaromatic, ornithine, or 2,3-diaminopropionic acid, carboxylic acids or any of the many well-known activated derivatives such as acid chlorides, active esters, active azolides and related derivatives, lysine, and ornithine.

In some cases, IL-10 comprises can comprise one or more naturally occurring L-amino acids, synthetic L-amino acids, and/or D-enantiomers of an amino acid. The IL-10 polypeptide can comprise one or more of the following amino acids: ω-aminodecanoic acid, ω-aminotetradecanoic acid, cyclohexylalanine, α,γ-diaminobutyric acid, α,β-diaminopropionic acid, δ-amino valeric acid, t-butylalanine, t-butylglycine, N-methylisoleucine, phenylglycine, cyclohexylalanine, norleucine, naphthylalanine, ornithine, citrulline, 4-chlorophenylalanine, 2-fluorophenylalanine, pyridylalanine 3-benzothienyl alanine, hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine(sarcosine), 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, F-amino hexanoic acid, ω-aminohexanoic acid, ω-aminoheptanoic acid, ω-aminooctanoic acid, and 2,3-diaminobutyric acid.

IL-10 can comprise a cysteine residue or a cysteine which can act as linker to another peptide via a disulfide linkage or to provide for cyclization of the IL-10 polypeptide. Methods of introducing a cysteine or cysteine analog are known in the art; see, e.g., U.S. Pat. No. 8,067,532. An IL-10 polypeptide can be cyclized. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)n-CO— or —(CH2)n-C6H4-CO—) with functional groups which allow for the introduction of a bridge. Some examples are disulfides, disulfide mimetics such as the —(CH2)n-carba bridge, thioacetal, thioether bridges (cystathionine or lanthionine) and bridges containing esters and ethers.

The IL-10 can be substituted with an N-alkyl, aryl, or backbone crosslinking to construct lactams and other cyclic structures, C-terminal hydroxymethyl derivatives, o-modified derivatives, N-terminally modified derivatives including substituted amides such as alkylamides and hydrazides. In some cases, an IL-10 polypeptide is a retroinverso analog.

IL-10 can be IL-10 can be native protein, peptide fragment IL-10, or modified peptide, having at least a portion of the biological activity of native IL-10. IL-10 can be modified to improve intracellular uptake. One such modification can be attachment of a protein transduction domain. The protein transduction domain can be attached to the C-terminus of the IL-10. Alternatively, the protein transduction domain can be attached to the N-terminus of the IL-10. The protein transduction domain can be attached to IL-10 via covalent bond. The protein transduction domain can be chosen from any of the sequences listed in Table 9.

TABLE 9
Exemplary protein transduction domains
Amino Acid Sequence

YGRKKRRQRRR (SEQ ID NO: 8)
RRQRRTSKLMKR (SEQ ID NO: 9)
GWTLNSAGYLLGKINLKALAALAKKIL (SEQ ID NO: 10)
KALAWEAKLAKALAKALAKHLAKALAKALKCEA
(SEQ ID NO: 11)
RQIKIWFQNRRMKWKK (SEQ ID NO: 12)
YGRKKRRQRRR (SEQ ID NO: 13)
RKKRRQRRR (SEQ ID NO: 14)
YGRKKRRQRRR (SEQ ID NO: 15)
RKKRRQRR (SEQ ID NO: 16)
YARAAARQARA (SEQ ID NO: 17)
THRLPRRRRRR (SEQ ID NO: 18)
GGRRARRRRRR (SEQ ID NO: 19)

### BPXTEN Structural Configurations and Properties

The BP of the subject compositions are not limited to native, full-length polypeptides, but also include recombinant versions as well as biologically and/or pharmacologically active variants or fragments thereof. For example, it will be appreciated that various amino acid substitutions can be made in the GP to create variants without departing from the spirit of the invention with respect to the biological activity or pharmacologic properties of the BP. Examples of conservative substitutions for amino acids in polypeptide sequences are shown in Table 4. However, in embodiments of the BPXTEN in which the sequence identity of the BP is less than 100% compared to a specific sequence disclosed herein, the invention contemplates substitution of any of the other 19 natural L-amino acids for a given amino acid residue of the given BP, which may be at any position within the sequence of the BP, including adjacent amino acid residues. If any one substitution results in an undesirable change in biological activity, then one of the alternative amino acids can be employed and the construct evaluated by the methods described herein, or using any of the techniques and guidelines for conservative and non-conservative mutations set forth, for instance, in U.S. Pat. No. 5,364,934, the contents of which is incorporated by reference in its entirety, or using methods generally known to those of skill in the art. In addition, variants can also include, for instance, polypeptides wherein one or more amino acid residues are added or deleted at the N- or C-terminus of the full-length native amino acid sequence of a BP that retains at least a portion of the biological activity of the native peptide.

TABLE 4
Exemplary conservative amino acid substitutions

| Original Residue | Exemplary Substitutions |
|---|---|
| Ala (A) | val; leu; ile |
| Arg (R) | lys; gin; asn |
| Asn (N) | gin; his; Iys; arg |
| Asp (D) | glu |
| Cys (C) | ser |
| Gln (Q) | asn |
| Glu (E) | asp |
| Gly (G) | pro |
| His (H) | asn: gin: Iys: arg |
| xIle (I) | leu; val; met; ala; phe: norleucine |
| Leu (L) | norleucine: ile: val; met; ala: phe |
| Lys (K) | arg: gin: asn |
| Met (M) | leu; phe; ile |
| Phe (F) | leu: val: ile; ala |
| Pro (P) | gly |
| Ser (S) | thr |
| Thr (T) | ser |
| Trp (W) | tyr |
| Tyr(Y) | trp: phe: thr: ser |
| Val (V) | ile; leu; met; phe; ala; norleucine |

### BPXTEN Fusion Protein Configurations

The invention provides BPXTEN fusion protein compositions comprising BP linked to one or more XTEN polypeptides useful for preventing, treating, mediating, or ameliorating a disease, disorder or condition related to glucose homeostasis, insulin resistance, or obesity. In some cases, the BPXTEN is a monomeric fusion protein with a BP linked to one or more XTEN polypeptides. In other cases, the BPXTEN composition can include two BP molecules linked to one or more XTEN polypeptides. The invention contemplates BPXTEN comprising, but not limited to BP selected from Table 3 or Table A (or fragments or sequence variants thereof), and XTEN selected from Tables 2a-2b or sequence variants thereof. In some cases, at least a portion of the biological activity of the respective BP is retained by the intact BPXTEN. In other cases, the BP component either becomes biologically active or has an increase in activity upon its release from the XTEN by cleavage of an optional cleavage sequence incorporated within spacer sequences into the BPXTEN, described more fully below.

In some embodiments, the BPXTEN fusion protein composition comprises (a) an XTEN (such as one disclosed herein) and (b) a cytokine linked to the XTEN.

In one embodiment of the BPXTEN composition, the invention provides a fusion protein of formula I:

(BP)-(S)ₓ-(XTEN) I

wherein independently for each occurrence, BP is a is a biologically active protein as described hereinabove; S is a spacer sequence having between 1 to about 50 amino acid residues that can optionally include a cleavage sequence (as described more fully below); x is either 0 or 1; and XTEN is an extended recombinant polypeptide as described hereinabove. The embodiment has particular utility where the BP requires a free N-terminus for desired biological activity, or where linking of the C-terminus of the BP to the fusion protein reduces biological activity and it is desired to reduce the biological activity and/or side effects of the administered BPXTEN.

In another embodiment of the BPXTEN composition, the invention provides a fusion protein of formula II (components as described above):

(XTEN)-(S)ₓ-(BP) II

wherein independently for each occurrence, BP is a is a biologically active protein as described hereinabove; S is a spacer sequence having between 1 to about 50 amino acid residues that can optionally include a cleavage sequence (as described more fully below); x is either 0 or 1; and XTEN is an extended recombinant polypeptide as described hereinabove. The embodiment has particular utility where the BP requires a free C-terminus for desired biological activity, or where linking of the N-terminus of the BP to the fusion protein reduces biological activity and it is desired to reduce the biological activity and/or side effects of the administered BPXTEN.

Thus, the BPXTEN having a single BP and a single XTEN can have at least the following permutations of configurations, each listed in an N- to C-terminus orientation: BP-XTEN; XTEN-BP; BP-S-XTEN; or XTEN-S-BP.

In another embodiment, the invention provides an isolated fusion protein, wherein the fusion protein is of formula III:

(BP)-(S)ₓ-(XTEN)-(S)_{y}-(BP)-(S)_{z}-(XTEN)_{z} III

wherein independently for each occurrence, BP is a is a biologically active protein as described hereinabove; S is a spacer sequence having between 1 to about 50 amino acid residues that can optionally include a cleavage sequence (as described more fully below); x is either 0 or 1; y is either 0 or 1; z is either 0 or 1; and XTEN is an extended recombinant polypeptide as described hereinabove.

In another embodiment, the invention provides an isolated fusion protein, wherein the fusion protein is of formula IV (components as described above):

(XTEN)ₓ-(S)_{y}-(BP)-(S)_{z}-(XTEN)-(BP) IV

In another embodiment, the invention provides an isolated fusion protein, wherein the fusion protein is of formula V (components as described above):

(BP)ₓ-(S)ₓ-(BP)-(S)_{y}-(XTEN) V

In another embodiment, the invention provides an isolated fusion protein, wherein the fusion protein is of formula VI (components as described above):

(XTEN)-(S)ₓ-(BP)-(S)_{y}-(BP) VI

In another embodiment, the invention provides an isolated fusion protein, wherein the fusion protein is of formula VII (components as described above):

(XTEN)-(S)ₓ-(BP)-(S)_{y}-(BP)-(XTEN) VII

In some cases, the BP can comprise a first fragment and a second cytokine fragment, and the XTEN is positioned between the first fragment and the second fragment. When desired, the BP can be cytokine. In some cases, the cytokine can be IL-10.

In the foregoing embodiments of fusion proteins of formulas I-VII, administration of a therapeutically effective dose of a fusion protein of an embodiment to a subject in need thereof can result in a gain in time of at least two-fold, or at least three-fold, or at least four-fold, or at least five-fold or more spent within a therapeutic window for the fusion protein compared to the corresponding BP not linked to the XTEN of and administered at a comparable dose to a subject.

Any spacer sequence group is optional in the fusion proteins encompassed by the invention. The spacer may be provided to enhance expression of the fusion protein from a host cell or to decrease steric hindrance such that the BP component may assume its desired tertiary structure and/or interact appropriately with its target molecule. For spacers and methods of identifying desirable spacers, see, for example, George, et al. (2003) Protein Engineering 15:871-879, specifically incorporated by reference herein. In one embodiment, the spacer comprises one or more peptide sequences that are between 1-50 amino acid residues in length, or about 1-25 residues, or about 1-10 residues in length. Spacer sequences, exclusive of cleavage sites, can comprise any of the 20 natural L amino acids, and will preferably comprise hydrophilic amino acids that are sterically unhindered that can include, but not be limited to, glycine (G), alanine (A), serine (S), threonine (T), glutamate (E) and proline (P). In some cases, the spacer can be polyglycines or polyalanines, or is predominately a mixture of combinations of glycine and alanine residues. The spacer polypeptide exclusive of a cleavage sequence is largely to substantially devoid of secondary structure. In one embodiment, one or both spacer sequences in a BPXTEN fusion protein composition may each further contain a cleavage sequence, which may be identical or may be different, wherein the cleavage sequence may be acted on by a protease to release the BP from the fusion protein.

In some cases, the incorporation of the cleavage sequence into the BPXTEN is designed to permit release of a BP that becomes active or more active upon its release from the XTEN. The cleavage sequences are located sufficiently close to the BP sequences, generally within 18, or within 12, or within 6, or within 2 amino acids of the BP sequence terminus, such that any remaining residues attached to the BP after cleavage do not appreciably interfere with the activity (e.g., such as binding to a receptor) of the BP, yet provide sufficient access to the protease to be able to effect cleavage of the cleavage sequence. In some embodiments, the cleavage site is a sequence that can be cleaved by a protease endogenous to the mammalian subject such that the BPXTEN can be cleaved after administration to a subject. In such cases, the BPXTEN can serve as a prodrug or a circulating depot for the BP. Examples of cleavage sites contemplated by the invention include, but are not limited to, a polypeptide sequence cleavable by a mammalian endogenous protease selected from FXIa, FXIIa, kallikrein, FVIIa, FIXa, FXa, FIIa (thrombin), Elastase-2, granzyme B, MMP-12, MMP-13, MMP-17 or MMP-20, or by non-mammalian proteases such as TEV, enterokinase, PreScission™ protease (rhinovirus 3C protease), and sortase A. Sequences known to be cleaved by the foregoing proteases are known in the art. Exemplary cleavage sequences and cut sites within the sequences are presented in Table 5, as well as sequence variants. For example, thrombin (activated clotting factor II) acts on the sequence LTPRSLLV (SEQ ID NO: 230) [Rawlings N. D., et al. (2008) Nucleic Acids Res., 36: D320], which would be cut after the arginine at position 4 in the sequence. Similarly, incorporation of other sequences into BPXTEN that are acted upon by endogenous proteases would provide for sustained release of BP that may, in certain cases, provide a higher degree of activity for the BP from the “prodrug” form of the BPXTEN.

In some cases, only the two or three amino acids flanking both sides of the cut site (four to six amino acids total) would be incorporated into the cleavage sequence. In other cases, the known cleavage sequence can have one or more deletions or insertions or one or two or three amino acid substitutions for any one or two or three amino acids in the known sequence, wherein the deletions, insertions or substitutions result in reduced or enhanced susceptibility but not an absence of susceptibility to the protease, resulting in an ability to tailor the rate of release of the BP from the XTEN. Exemplary substitutions are shown in Table 5.

TABLE 5
Protease Cleavage Sequences

| | | Exemplary | | |
|---|---|---|---|---|
| Protease Acting | SEQ ID | Cleavage | SEQ ID | |
| Upon Sequence | NO | Sequence | NO | Minimal Cut Site* |
| FXIa | 274 | KLTR↓VVGG | 869 | KD/FL/T/R↓VA/VE/GT/GV |
| FXIIa | 275 | TMTR↓IVGG | NA | NA |
| Kallikrein | 276 | SPFR↓STGG | 870 | -/-/FL/RY↓SR/RT/-/- |
| FVIIa | 277 | LQVR↓IVGG | NA | NA |
| FIXa | 278 | PLGR↓IVGG | 871 | -/-/G/R↓-/-/-/- |
| FXa | 279 | IEGR↓TVGG | 872 | IA/E/GFP/R↓STI/VFS/-/G |
| FIIa (thrombin) | 280 | LTPR↓SLLV | 873 | -/-/PLA/R↓SAG/-/-/- |
| Elastase-2 | 281 | LGPV↓SGVP | 874 | -/-/-/VIAT↓-/-/-/- |
| Granzyme-B | 282 | VAGD↓SLEE | 875 | V/-/-/D↓-/-/-/- |
| MMP-12 | 283 | GPAG↓LGGA | 876 | G/PA/-/G↓L/-/G/- |
| MMP-13 | 284 | GPAG↓LRGA | 877 | G/P/-/G↓L/-/GA/- |
| MMP-17 | 285 | APLG↓LRLR | 878 | -/PS/-/-↓LQ/-/LT/- |
| MMP-20 | 286 | PALP↓LVAQ | NA | NA |
| TEV | 287 | ENLYFQ↓G | 879 | ENLYFQ↓/GS |
| Enterokinase | 288 | DDDK↓IVGG | 288 | DDDK↓IVGG |
| Protease 3C | 867 | LEVLFQ↓GP | 867 | LEVLFQ↓GP |
| (PreScission ™) | | | | |
| Sortase A | 868 | LPKT↓GSES | 880 | L/P/KEAD/T↓G/-/EKS/S |

↓indicates cleavage site
NA: not applicable
*the listing of multiple amino acids before, between, or after a slash indicate alternative amino acids that can be substituted at the position; ″-″ indicates that any amino acid may be substituted for the corresponding amino acid indicated in the middle column

In another aspect, the disclosure provides fusion protein comprising multiple release segment (RS) wherein each RS sequence is selected from the group of sequences set forth in Table 6 and the RS are linked to each other by 1 to 6 amino acids selected from glycine, serine, alanine, and threonine. In one embodiment, the fusion protein comprises a first RS and a second RS different from the first RS wherein each RS sequence is selected from the group of sequences set forth in Table 6 and the RS are linked to each other by 1 to 6 amino acids selected from glycine, serine, alanine, and threonine. In another embodiment, the fusion protein comprises a first RS, a second RS different from the first RS, and a third RS different from the first and the second RS wherein each sequence is selected from the group of sequences set forth in Table 6 and the first and the second and the third RS are linked to each other by 1 to 6 amino acids selected from glycine, serine, alanine, and threonine. It is specifically intended that the multiple RS of the fusion protein can be concatenated to form a sequence that can be cleaved by multiple proteases at different rates or efficiency of cleavage. In another embodiment, the disclosure provides fusion protein comprising an RS1 and an RS2 selected from the group of sequences set forth in Tables 6 and 7 and an XTEN1 and XTEN2 selected from current disclosure wherein the RS1 is fused between the XTEN1 and the binding moieties and the RS2 is fused between the XTEN2 and the binding moieties. It is contemplated that such compositions would be more readily cleaved by diseased target tissues that express multiple proteases, compared with healthy tissues or when in the normal circulation, with the result that the resulting fragments bearing the binding moieties would more readily penetrate the target tissue; e.g., a tumor, and have an enhanced ability to bind and link the target cell and the effector cell (or just the target cell in the case of fusion protein designed with a single binding moiety. In some embodiments, where the composition of this disclosure (such as a fusion protein) comprises a release segment, the release segment (RS) can have at least 82%, at least 88%, at least 94%, or 100% sequence identity to a sequence selected from the sequences set forth in Tables 6-7. In some embodiments, the composition of this disclosure (such as a fusion protein) can have a structural arrangement, from N- to C-terminus of XTEN-RS-cytokine or cytokine-RS-XTEN.

TABLE 6
Release Segment Sequences.

| Name | Construct ID | Amino Acid Sequence |
|---|---|---|
| BSRS-4 | AC1602 | LAGRSDNHSPLGLAGS (SEQ ID NO: 20) |
| BSRS-5 | AC1603 | LAGRSDNHVPLSLSMG (SEQ ID NO: 21) |
| BSRS-6 | AC1604 | LAGRSDNHEPLELVAG (SEQ ID NO: 22) |
| BSRS-A1-1 | AC1605 | ASGRSTNAGPSGLAGP (SEQ ID NO: 23) |
| BSRS-A2-1 | AC1606 | ASGRSTNAGPQGLAGQ (SEQ ID NO: 24) |
| BSRS-A3-1 | AC1607 | ASGRSTNAGPPGLTGP (SEQ ID NO: 25) |
| VP-1 | AC1608 | ASSRGTNAGPAGLTGP (SEQ ID NO: 26) |
| RSR-1752 | AC1609 | ASSRTTNTGPSTLTGP (SEQ ID NO: 27) |
| RSR-1512 | AC1610 | AAGRSDNGTPLELVAP (SEQ ID NO: 28) |
| RSR-1517 | AC1611 | EAGRSANHEPLGLVAT (SEQ ID NO: 29) |
| VP-2 | AC1612 | ASGRGTNAGPAGLTGP (SEQ ID NO: 30) |
| RSR-1018 | AC1613 | LFGRNDNHEPLELGGG (SEQ ID NO: 31) |
| RSR-1053 | AC1614 | TAGRSDNLEPLGLVFG (SEQ ID NO: 32) |
| RSR-1059 | AC1615 | LDGRSDNFHPPELVAG (SEQ ID NO: 33) |
| RSR-1065 | AC1616 | LEGRSDNEEPENLVAG (SEQ ID NO: 34) |
| RSR-1167 | AC1617 | LKGRSDNNAPLALVAG (SEQ ID NO: 35) |
| RSR-1201 | AC1618 | VYSRGTNAGPHGLTGR (SEQ ID NO: 36) |
| RSR-1218 | AC1619 | ANSRGTNKGFAGLIGP (SEQ ID NO: 37) |
| RSR-1226 | AC1620 | ASSRLINEAPAGLTIP (SEQ ID NO: 38) |
| RSR-1254 | AC1621 | DOSRGTNAGPEGLTDP (SEQ ID NO: 39) |
| RSR-1256 | AC1622 | ESSRGTNIGQGGLTGP (SEQ ID NO: 40) |
| RSR-1261 | AC1623 | SSSRGTNQDPAGLTIP (SEQ ID NO: 41) |
| RSR-1293 | AC1624 | ASSRGONHSPMGLTGP (SEQ ID NO: 42) |
| RSR-1309 | AC1625 | AYSRGPNAGPAGLEGR (SEQ ID NO: 43) |
| RSR-1326 | AC1626 | ASERGNNAGPANLTGF (SEQ ID NO: 44) |
| RSR-1345 | AC1627 | ASHRGTNPKPAILTGP (SEQ ID NO: 45) |
| RSR-1354 | AC1628 | MSSRRTNANPAQLTGP (SEQ ID NO: 46) |
| RSR-1426 | AC1629 | GAGRTDNHEPLELGAA (SEQ ID NO: 47) |
| RSR-1478 | AC1630 | LAGRSENTAPLELTAG (SEQ ID NO: 48) |
| RSR-1479 | AC1631 | LEGRPDNHEPLALVAS (SEQ ID NO: 49) |
| RSR-1496 | AC1632 | LSGRSDNEEPLALPAG (SEQ ID NO: 50) |
| RSR-1508 | AC1633 | EAGRTDNHEPLELSAP (SEQ ID NO: 51) |
| RSR-1513 | AC1634 | EGGRSDNHGPLELVSG (SEQ ID NO: 52) |
| RSR-1516 | AC1635 | LSGRSDNEAPLELEAG (SEQ ID NO: 53) |
| RSR-1524 | AC1636 | LGGRADNHEPPELGAG (SEQ ID NO: 54) |
| RSR-1622 | AC1637 | PPSRGTNAEPAGLIGE (SEQ ID NO: 55) |
| RSR-1629 | AC1638 | ASTRGENAGPAGLEAP (SEQ ID NO: 56) |
| RSR-1664 | AC1639 | ESSRGTNGAPEGLTGP (SEQ ID NO: 57) |
| RSR-1667 | AC1640 | ASSRATNESPAGLTGE (SEQ ID NO: 58) |
| RSR-1709 | AC1641 | ASSRGENPPPGGLTGP (SEQ ID NO: 59) |
| RSR-1712 | AC1642 | AASRGTNTGPAELTGS (SEQ ID NO: 60) |
| RSR-1727 | AC1643 | AGSRTTNAGPGGLEGP (SEQ ID NO: 61) |
| RSR-1754 | AC1644 | APSRGENAGPATLIGA (SEQ ID NO: 62) |
| RSR-1819 | AC1645 | ESGRAANTGPPTLTAP (SEQ ID NO: 63) |
| RSR-1832 | AC1646 | NPGRAANEGPPGLPGS (SEQ ID NO: 64) |
| RSR-1855 | AC1647 | ESSRAANLTPPELTGP (SEQ ID NO: 65) |
| RSR-1911 | AC1648 | ASGRAANETPPGLTGA (SEQ ID NO: 66) |
| RSR-1929 | AC1649 | NSGRGENLGAPGLIGT (SEQ ID NO: 67) |
| RSR-1951 | AC1650 | TTGRAANLTPAGLTGP (SEQ ID NO: 68) |
| RSR-2295 | AC1761 | EAGRSANHTPAGLTGP (SEQ ID NO: 69) |
| RSR-2298 | AC1762 | ESGRAANTTPAGLTGP (SEQ ID NO: 70) |
| RSR-2038 | AC1679 | TTGRATEAANLTPAGLTGP (SEQ ID NO: 71) |
| RSR-2072 | AC1680 | TTGRAEEAANLTPAGLTGP (SEQ ID NO: 72) |
| RSR-2089 | AC1681 | TTGRAGEAANLTPAGLTGP (SEQ ID NO: 73) |
| RSR-2302 | AC1682 | TTGRATEAANATPAGLTGP (SEQ ID NO: 74) |
| RSR-3047 | AC1697 | TTGRAGEAEGATSAGATGP (SEQ ID NO: 75) |
| RSR-3052 | AC1698 | TTGEAGEAANATSAGATGP (SEQ ID NO: 76) |
| RSR-3043 | AC1699 | TTGEAGEAAGLTPAGLTGP (SEQ ID NO: 77) |
| RSR-3041 | AC1700 | TTGAAGEAANATPAGLTGP (SEQ ID NO: 78) |
| RSR-3044 | AC1701 | TTGRAGEAAGLTPAGLTGP (SEQ ID NO: 79) |
| RSR-3057 | AC1702 | TTGRAGEAANATSAGATGP (SEQ ID NO: 80) |
| RSR-3058 | AC1703 | TTGEAGEAAGATSAGATGP (SEQ ID NO: 81) |
| RSR-2485 | AC1763 | ESGRAANTEPPELGAG (SEQ ID NO: 82) |
| RSR-2486 | AC1764 | ESGRAANTAPEGLTGP (SEQ ID NO: 83) |
| RSR-2488 | AC1688 | EPGRAANHEPSGLTEG (SEQ ID NO: 84) |
| RSR-2599 | AC1706 | ESGRAANHTGAPPGGLTGP (SEQ ID NO: 85) |
| RSR-2706 | AC1716 | TTGRTGEGANATPGGLTGP (SEQ ID NO: 86) |
| RSR-2707 | AC1717 | RTGRSGEAANETPEGLEGP (SEQ ID NO: 87) |
| RSR-2708 | AC1718 | RTGRTGESANETPAGLGGP (SEQ ID NO: 88) |
| RSR-2709 | AC1719 | STGRTGEPANETPAGLSGP (SEQ ID NO: 89) |
| RSR-2710 | AC1720 | TTGRAGEPANATPTGLSGP (SEQ ID NO: 90) |
| RSR-2711 | AC1721 | RTGRPGEGANATPTGLPGP (SEQ ID NO: 91) |
| RSR-2712 | AC1722 | RTGRGGEAANATPSGLGGP (SEQ ID NO: 92) |
| RSR-2713 | AC1723 | STGRSGESANATPGGLGGP (SEQ ID NO: 93) |
| RSR-2714 | AC1724 | RTGRTGEEANATPAGLPGP (SEQ ID NO: 94) |
| RSR-2715 | AC1725 | ATGRPGEPANTTPEGLEGP (SEQ ID NO: 95) |
| RSR-2716 | AC1726 | STGRSGEPANATPGGLTGP (SEQ ID NO: 96) |
| RSR-2717 | AC1727 | PTGRGGEGANTTPTGLPGP (SEQ ID NO: 97) |
| RSR-2718 | AC1728 | PTGRSGEGANATPSGLTGP (SEQ ID NO: 98) |
| RSR-2719 | AC1729 | TTGRASEGANSTPAPLTEP (SEQ ID NO: 99) |
| RSR-2720 | AC1730 | TYGRAAEAANTTPAGLTAP (SEQ ID NO: 100) |
| RSR-2721 | AC1731 | TTGRATEGANATPAELTEP (SEQ ID NO: 101) |
| RSR-2722 | AC1732 | TVGRASEEANTTPASLTGP (SEQ ID NO: 102) |
| RSR-2723 | AC1733 | TTGRAPEAANATPAPLTGP (SEQ ID NO: 103) |
| RSR-2724 | AC1734 | TWGRATEPANATPAPLTSP (SEQ ID NO: 104) |
| RSR-2725 | AC1735 | TVGRASESANATPAELTSP (SEQ ID NO: 105) |
| RSR-2726 | AC1736 | TVGRAPEGANSTPAGLTGP (SEQ ID NO: 106) |
| RSR-2727 | AC1737 | TWGRATEAPNLEPATLTTP (SEQ ID NO: 107) |
| RSR-2728 | AC1738 | TTGRATEAPNLTPAPLTEP (SEQ ID NO: 108) |
| RSR-2729 | AC1739 | TOGRATEAPNLSPAALTSP (SEQ ID NO: 109) |
| RSR-2730 | AC1740 | TOGRAAEAPNLTPATLTAP (SEQ ID NO: 110) |
| RSR-2731 | AC1741 | TSGRAPEATNLAPAPLTGP (SEQ ID NO: 111) |
| RSR-2732 | AC1742 | TOGRAAEAANLTPAGLTEP (SEQ ID NO: 112) |
| RSR-2733 | AC1743 | TTGRAGSAPNLPPTGLTTP (SEQ ID NO: 113) |
| RSR-2734 | AC1744 | TTGRAGGAENLPPEGLTAP (SEQ ID NO: 114) |
| RSR-2735 | AC1745 | TTSRAGTATNLTPEGLTAP (SEQ ID NO: 115) |
| RSR-2736 | AC1746 | TTGRAGTATNLPPSGLTTP (SEQ ID NO: 116) |
| RSR-2737 | AC1747 | TTARAGEAENLSPSGLTAP (SEQ ID NO: 117) |
| RSR-2738 | AC1748 | TTGRAGGAGNLAPGGLTEP (SEQ ID NO: 118) |
| RSR-2739 | AC1749 | TTGRAGTATNLPPEGLTGP (SEQ ID NO: 119) |
| RSR-2740 | AC1750 | TTGRAGGAANLAPTGLTEP (SEQ ID NO: 120) |
| RSR-2741 | AC1751 | TTGRAGTAENLAPSGLTTP (SEQ ID NO: 121) |
| RSR-2742 | AC1752 | TTGRAGSATNLGPGGLTGP (SEQ ID NO: 122) |
| RSR-2743 | AC1753 | TTARAGGAENLTPAGLTEP (SEQ ID NO: 123) |
| RSR-2744 | AC1754 | TTARAGSAENLSPSGLTGP (SEQ ID NO: 124) |
| RSR-2745 | AC1755 | TTARAGGAGNLAPEGLTTP (SEQ ID NO: 125) |
| RSR-2746 | AC1756 | TTSRAGAAENLTPTGLTGP (SEQ ID NO: 126) |
| RSR-2747 | AC1757 | TYGRTTTPGNEPPASLEAE (SEQ ID NO: 127) |
| RSR-2748 | AC1758 | TYSRGESGPNEPPPGLTGP (SEQ ID NO: 128) |
| RSR-2749 | AC1759 | AWGRTGASENETPAPLGGE (SEQ ID NO: 129) |
| RSR-2750 | AC1760 | RWGRAETTPNTPPEGLETE (SEQ ID NO: 130) |
| RSR-2751 | AC1765 | ESGRAANHTGAEPPELGAG (SEQ ID NO: 131) |
| RSR-2754 | AC1801 | TTGRAGEAANLTPAGLTES (SEQ ID NO: 132) |
| RSR-2755 | AC1802 | TTGRAGEAANLTPAALTES (SEQ ID NO: 133) |
| RSR-2756 | AC1803 | TTGRAGEAANLTPAPLTES (SEQ ID NO: 134) |
| RSR-2757 | AC1804 | TTGRAGEAANLTPEPLTES (SEQ ID NO: 135) |
| RSR-2758 | AC1805 | TTGRAGEAANLTPAGLTGA (SEQ ID NO: 136) |
| RSR-2759 | AC1806 | TTGRAGEAANLTPEGLTGA (SEQ ID NO: 137) |
| RSR-2760 | AC1807 | TTGRAGEAANLTPEPLTGA (SEQ ID NO: 138) |
| RSR-2761 | AC1808 | TTGRAGEAANLTPAGLTEA (SEQ ID NO: 139) |
| RSR-2762 | AC1809 | TTGRAGEAANLTPEGLTEA (SEQ ID NO: 140) |
| RSR-2763 | AC1810 | TTGRAGEAANLTPAPLTEA (SEQ ID NO: 141) |
| RSR-2764 | AC1811 | TTGRAGEAANLTPEPLTEA (SEQ ID NO: 142) |
| RSR-2765 | AC1812 | TTGRAGEAANLTPEPLTGP (SEQ ID NO: 143) |
| RSR-2766 | AC1813 | TTGRAGEAANLTPAGLTGG (SEQ ID NO: 144) |
| RSR-2767 | AC1814 | TTGRAGEAANLTPEGLTGG (SEQ ID NO: 145) |
| RSR-2768 | AC1815 | TTGRAGEAANLTPEALTGG (SEQ ID NO: 146) |
| RSR-2769 | AC1816 | TTGRAGEAANLTPEPLIGG (SEQ ID NO: 147) |
| RSR-2770 | AC1817 | TTGRAGEAANLTPAGLTEG (SEQ ID NO: 148) |
| RSR-2771 | AC1818 | TTGRAGEAANLTPEGLTEG (SEQ ID NO: 149) |
| RSR-2772 | AC1819 | TTGRAGEAANLTPAPLTEG (SEQ ID NO: 150) |
| RSR-2773 | AC1820 | TTGRAGEAANLTPEPLTEG (SEQ ID NO: 151) |

TABLE 7
Release Segment Sequences

| Name | Amino Acid Sequence | Name | Amino Acid Sequence |
|---|---|---|---|
| RSN-0001 | GSAPGSAGGYAELRMGGAIATSGSETP | RSC-0001 | GTAEAASASGGSAGGYAELRMGGAIPG |
| | GT (SEQ ID NO: 335) | | SP (SEQ ID NO: 583) |
| RSN-0002 | GSAPGTGGGYAPLRMGGGAATSGSETP | RSC-0002 | GTAEAASASGGTGGGYAPLRMGGGAPG |
| | GT (SEQ ID NO: 336) | | SP (SEQ ID NO: 584) |
| RSN-0003 | GSAPGAEGGYAALRMGGEIATSGSETP | RSC-0003 | GTAEAASASGGAEGGYAALRMGGEIPG |
| | GT (SEQ ID NO: 337) | | SP (SEQ ID NO: 585) |
| RSN-0004 | GSAPGGPGGYALLRMGGPAATSGSETP | RSC-0004 | GTAEAASASGGGPGGYALLRMGGPAPG |
| | GT (SEQ ID NO: 338) | | SP (SEQ ID NO: 586) |
| RSN-0005 | GSAPGEAGGYAFLRMGGSIATSGSETP | RSC-0005 | GTAEAASASGGEAGGYAFLRMGGSIPG |
| | GT (SEQ ID NO: 339) | | SP (SEQ ID NO: 587) |
| RSN-0006 | GSAPGPGGGYASLRMGGTAATSGSETP | RSC-0006 | GTAEAASASGGPGGGYASLRMGGTAPG |
| | GT (SEQ ID NO: 340) | | SP (SEQ ID NO: 588) |
| RSN-0007 | GSAPGSEGGYATLRMGGAIATSGSETP | RSC-0007 | GTAEAASASGGSEGGYATLRMGGAIPG |
| | GT (SEQ ID NO: 341) | | SP (SEQ ID NO: 589) |
| RSN-0008 | GSAPGTPGGYANLRMGGGAATSGSETP | RSC-0008 | GTAEAASASGGTPGGYANLRMGGGAPG |
| | GT (SEQ ID NO: 342) | | SP (SEQ ID NO: 590) |
| RSN-0009 | GSAPGASGGYAHLRMGGEIATSGSETP | RSC-0009 | GTAEAASASGGASGGYAHLRMGGEIPG |
| | GT (SEQ ID NO: 343) | | SP (SEQ ID NO: 591) |
| RSN-0010 | GSAPGGTGGYGELRMGGPAATSGSETP | RSC-0010 | GTAEAASASGGGTGGYGELRMGGPAPG |
| | GT (SEQ ID NO: 344) | | SP (SEQ ID NO: 592) |
| RSN-0011 | GSAPGEAGGYPELRMGGSIATSGSETP | RSC-0011 | GTAEAASASGGEAGGYPELRMGGSIPG |
| | GT (SEQ ID NO: 345) | | SP (SEQ ID NO: 593) |
| RSN-0012 | GSAPGPGGGYVELRMGGTAATSGSETP | RSC-0012 | GTAEAASASGGPGGGYVELRMGGTAPG |
| | GT (SEQ ID NO: 346) | | SP (SEQ ID NO: 594) |
| RSN-0013 | GSAPGSEGGYLELRMGGAIATSGSETP | RSC-0013 | GTAEAASASGGSEGGYLELRMGGAIPG |
| | GT (SEQ ID NO: 347) | | SP (SEQ ID NO: 595) |
| RSN-0014 | GSAPGTPGGYSELRMGGGAATSGSETP | RSC-0014 | GTAEAASASGGTPGGYSELRMGGGAPG |
| | GT (SEQ ID NO: 348) | | SP (SEQ ID NO: 596) |
| RSN-0015 | GSAPGASGGYTELRMGGEIATSGSETP | RSC-0015 | GTAEAASASGGASGGYTELRMGGEIPG |
| | GT (SEQ ID NO: 349) | | SP(SEQ ID NO: 597) |
| RSN-0016 | GSAPGGTGGYQELRMGGPAATSGSETP | RSC-0016 | GTAEAASASGGGTGGYQELRMGGPAPG |
| | GT (SEQ ID NO: 350) | | SP (SEQ ID NO: 598) |
| RSN-0017 | GSAPGEAGGYEELRMGGSIATSGSETP | RSC-0017 | GTAEAASASGGEAGGYEELRMGGSIPG |
| | GT (SEQ ID NO: 351) | | SP (SEQ ID NO: 599) |
| RSN-0018 | GSAPGPGIGPAELRMGGTAATSGSETP | RSC-0018 | GTAEAASASGGPGIGPAELRMGGTAPG |
| | GT (SEQ ID NO: 352) | | SP (SEQ ID NO: 600) |
| RSN-0019 | GSAPGSEIGAAELRMGGAIATSGSETP | RSC-0019 | GTAEAASASGGSEIGAAELRMGGAIPG |
| | GT (SEQ ID NO: 353) | | SP (SEQ ID NO: 601) |
| RSN-0020 | GSAPGTPIGSAELRMGGGAATSGSETP | RSC-0020 | GTAEAASASGGTPIGSAELRMGGGAPG |
| | GT (SEQ ID NO: 354) | | SP (SEQ ID NO: 602) |
| RSN-0021 | GSAPGASIGTAELRMGGEIATSGSETP | RSC-0021 | GTAEAASASGGASIGTAELRMGGEIPG |
| | GT (SEQ ID NO: 355) | | SP (SEQ ID NO: 603) |
| RSN-0022 | GSAPGGTIGNAELRMGGPAATSGSETP | RSC-0022 | GTAEAASASGGGTIGNAELRMGGPAPG |
| | GT (SEQ ID NO: 356) | | SP (SEQ ID NO: 604) |
| RSN-0023 | GSAPGEAIGQAELRMGGSIATSGSETP | RSC-0023 | GTAEAASASGGEAIGQAELRMGGSIPG |
| | GT (SEQ ID NO: 357) | | SP (SEQ ID NO: 605) |
| RSN-0024 | GSAPGPGGPYAELRMGGTAATSGSETP | RSC-0024 | GTAEAASASGGPGGPYAELRMGGTAPG |
| | GT (SEQ ID NO: 358) | | SP (SEQ ID NO: 606) |
| RSN-0025 | GSAPGSEGAYAELRMGGAIATSGSETP | RSC-0025 | GTAEAASASGGSEGAYAELRMGGAIPG |
| | GT (SEQ ID NO: 359) | | SP (SEQ ID NO: 607) |
| RSN-0026 | GSAPGTPGVYAELRMGGGAATSGSETP | RSC-0026 | GTAEAASASGGTPGVYAELRMGGGAPG |
| | GT (SEQ ID NO: 360) | | SP (SEQ ID NO: 608) |
| RSN-0027 | GSAPGASGLYAELRMGGEIATSGSETP | RSC-0027 | GTAEAASASGGASGLYAELRMGGEIPG |
| | GT (SEQ ID NO: 361) | | SP (SEQ ID NO: 609) |
| RSN-0028 | GSAPGGTGIYAELRMGGPAATSGSETP | RSC-0028 | GTAEAASASGGGTGIYAELRMGGPAPG |
| | GT (SEQ ID NO: 362) | | SP(SEQ ID NO: 610) |
| RSN-0029 | GSAPGEAGFYAELRMGGSIATSGSETP | RSC-0029 | GTAEAASASGGEAGFYAELRMGGSIPG |
| | GT (SEQ ID NO: 363) | | SP (SEQ ID NO: 611) |
| RSN-0030 | GSAPGPGGYYAELRMGGTAATSGSETP | RSC-0030 | GTAEAASASGGPGGYYAELRMGGTAPG |
| | GT (SEQ ID NO: 364) | | SP (SEQ ID NO: 612) |
| RSN-0031 | GSAPGSEGSYAELRMGGAIATSGSETP | RSC-0031 | GTAEAASASGGSEGSYAELRMGGAIPG |
| | GT (SEQ ID NO: 365) | | SP (SEQ ID NO: 613) |
| RSN-0032 | GSAPGTPGNYAELRMGGGAATSGSETP | RSC-0032 | GTAEAASASGGTPGNYAELRMGGGAPG |
| | GT (SEQ ID NO: 366) | | SP (SEQ ID NO: 614) |
| RSN-0033 | GSAPGASGEYAELRMGGEIATSGSETP | RSC-0033 | GTAEAASASGGASGEYAELRMGGEIPG |
| | GT (SEQ ID NO: 367) | | SP (SEQ ID NO: 615) |
| RSN-0034 | GSAPGGTGHYAELRMGGPAATSGSETP | RSC-0034 | GTAEAASASGGGTGHYAELRMGGPAPG |
| | GT (SEQ ID NO: 368) | | SP(SEQ ID NO: 616) |
| RSN-0035 | GSAPGEAGGYAEARMGGSIATSGSETP | RSC-0035 | GTAEAASASGGEAGGYAEARMGGSIPG |
| | GT (SEQ ID NO: 369) | | SP (SEQ ID NO: 617) |
| RSN-0036 | GSAPGPGGGYAEVRMGGTAATSGSETP | RSC-0036 | GTAEAASASGGPGGGYAEVRMGGTAPG |
| | GT (SEQ ID NO: 370) | | SP (SEQ ID NO: 618) |
| RSN-0037 | GSAPGSEGGYAEIRMGGAIATSGSETP | RSC-0037 | GTAEAASASGGSEGGYAEIRMGGAIPG |
| | GT (SEQ ID NO: 371) | | SP (SEQ ID NO: 619) |
| RSN-0038 | GSAPGTPGGYAEFRMGGGAATSGSETP | RSC-0038 | GTAEAASASGGTPGGYAEFRMGGGAPG |
| | GT (SEQ ID NO: 372) | | SP (SEQ ID NO: 620) |
| RSN-0039 | GSAPGASGGYAEYRMGGEIATSGSETP | RSC-0039 | GTAEAASASGGASGGYAEYRMGGEIPG |
| | GT (SEQ ID NO: 373) | | SP (SEQ ID NO: 621) |
| RSN-0040 | GSAPGGTGGYAESRMGGPAATSGSETP | RSC-0040 | GTAEAASASGGGTGGYAESRMGGPAPG |
| | GT (SEQ ID NO: 374) | | SP (SEQ ID NO: 622) |
| RSN-0041 | GSAPGEAGGYAETRMGGSIATSGSETP | RSC-0041 | GTAEAASASGGEAGGYAETRMGGSIPG |
| | GT (SEQ ID NO: 375) | | SP (SEQ ID NO: 623) |
| RSN-0042 | GSAPGPGGGYAELAMGGTRATSGSETP | RSC-0042 | GTAEAASASGGPGGGYAELAMGGTRPG |
| | GT (SEQ ID NO: 376) | | SP (SEQ ID NO: 624) |
| RSN-0043 | GSAPGSEGGYAELVMGGARATSGSETP | RSC-0043 | GTAEAASASGGSEGGYAELVMGGARPG |
| | GT (SEQ ID NO: 377) | | SP (SEQ ID NO: 625) |
| RSN-0044 | GSAPGTPGGYAELLMGGGRATSGSETP | RSC-0044 | GTAEAASASGGTPGGYAELLMGGGRPG |
| | GT (SEQ ID NO: 378) | | SP (SEQ ID NO: 626) |
| RSN-0045 | GSAPGASGGYAELIMGGERATSGSETP | RSC-0045 | GTAEAASASGGASGGYAELIMGGERPG |
| | GT (SEQ ID NO: 379) | | SP (SEQ ID NO: 627) |
| RSN-0046 | GSAPGGTGGYAELWMGGPRATSGSETP | RSC-0046 | GTAEAASASGGGTGGYAELWMGGPRPG |
| | GT (SEQ ID NO: 380) | | SP (SEQ ID NO: 628) |
| RSN-0047 | GSAPGEAGGYAELSMGGSRATSGSETP | RSC-0047 | GTAEAASASGGEAGGYAELSMGGSRPG |
| | GT (SEQ ID NO: 381) | | SP (SEQ ID NO: 629) |
| RSN-0048 | GSAPGPGGGYAELTMGGTRATSGSETP | RSC-0048 | GTAEAASASGGPGGGYAELTMGGTRPG |
| | GT (SEQ ID NO: 382) | | SP (SEQ ID NO: 630) |
| RSN-0049 | GSAPGSEGGYAELQMGGARATSGSETP | RSC-0049 | GTAEAASASGGSEGGYAELQMGGARPG |
| | GT (SEQ ID NO: 383) | | SP (SEQ ID NO: 631) |
| RSN-0050 | GSAPGTPGGYAELNMGGGRATSGSETP | RSC-0050 | GTAEAASASGGTPGGYAELNMGGGRPG |
| | GT (SEQ ID NO: 384) | | SP (SEQ ID NO: 632) |
| RSN-0051 | GSAPGASGGYAELEMGGERATSGSETP | RSC-0051 | GTAEAASASGGASGGYAELEMGGERPG |
| | GT (SEQ ID NO: 385) | | SP (SEQ ID NO: 633) |
| RSN-0052 | GSAPGGTGGYAELRPGGPIATSGSETP | RSC-0052 | GTAEAASASGGGTGGYAELRPGGPIPG |
| | GT (SEQ ID NO: 386) | | SP (SEQ ID NO: 634) |
| RSN-0053 | GSAPGEAGGYAELRAGGSAATSGSETP | RSC-0053 | GTAEAASASGGEAGGYAELRAGGSAPG |
| | GT (SEQ ID NO: 387) | | SP (SEQ ID NO: 635) |
| RSN-0054 | GSAPGPGGGYAELRLGGTIATSGSETP | RSC-0054 | GTAEAASASGGPGGGYAELRLGGTIPG |
| | GT (SEQ ID NO: 388) | | SP (SEQ ID NO: 636) |
| RSN-0055 | GSAPGSEGGYAELRIGGAAATSGSETP | RSC-0055 | GTAEAASASGGSEGGYAELRIGGAAPG |
| | GT (SEQ ID NO: 389) | | SP (SEQ ID NO: 637) |
| RSN-0056 | GSAPGTPGGYAELRSGGGIATSGSETP | RSC-0056 | GTAEAASASGGTPGGYAELRSGGGIPG |
| | GT (SEQ ID NO: 390) | | SP (SEQ ID NO: 638) |
| RSN-0057 | GSAPGASGGYAELRNGGEAATSGSETP | RSC-0057 | GTAEAASASGGASGGYAELRNGGEAPG |
| | GT (SEQ ID NO: 391) | | SP (SEQ ID NO: 639) |
| RSN-0058 | GSAPGGTGGYAELRQGGPIATSGSETP | RSC-0058 | GTAEAASASGGGTGGYAELRQGGPIPG |
| | GT (SEQ ID NO: 392) | | SP (SEQ ID NO: 640) |
| RSN-0059 | GSAPGEAGGYAELRDGGSAATSGSETP | RSC-0059 | GTAEAASASGGEAGGYAELRDGGSAPG |
| | GT (SEQ ID NO: 393) | | SP (SEQ ID NO: 641) |
| RSN-0060 | GSAPGPGGGYAELREGGTIATSGSETP | RSC-0060 | GTAEAASASGGPGGGYAELREGGTIPG |
| | GT (SEQ ID NO: 394) | | SP (SEQ ID NO: 642) |
| RSN-0061 | GSAPGSEGGYAELRHGGAAATSGSETP | RSC-0061 | GTAEAASASGGSEGGYAELRHGGAAPG |
| | GT (SEQ ID NO: 395 | | SP (SEQ ID NO: 643) |
| RSN-0062 | GSAPGTPGGYAELRMPGGIATSGSETP | RSC-0062 | GTAEAASASGGTPGGYAELRMPGGIPG |
| | GT (SEQ ID NO: 396) | | SP (SEQ ID NO: 644) |
| RSN-0063 | GSAPGASGGYAELRMAGEAATSGSETP | RSC-0063 | GTAEAASASGGASGGYAELRMAGEAPG |
| | GT (SEQ ID NO: 397) | | SP (SEQ ID NO: 645) |
| RSN-0064 | GSAPGGTGGYAELRMVGPIATSGSETP | RSC-0064 | GTAEAASASGGGTGGYAELRMVGPIPG |
| | GT (SEQ ID NO: 398) | | SP (SEQ ID NO: 646) |
| RSN-0065 | GSAPGEAGGYAELRMLGSAATSGSETP | RSC-0065 | GTAEAASASGGEAGGYAELRMLGSAPG |
| | GT (SEQ ID NO: 399) | | SP (SEQ ID NO: 647) |
| RSN-0066 | GSAPGPGGGYAELRMIGTIATSGSETP | RSC-0066 | GTAEAASASGGPGGGYAELRMIGTIPG |
| | GT (SEQ ID NO: 400) | | SP(SEQ ID NO: 648) |
| RSN-0067 | GSAPGSEGGYAELRMYGAIATSGSETP | RSC-0067 | GTAEAASASGGSEGGYAELRMYGAIPG |
| | GT (SEQ ID NO: 401) | | SP (SEQ ID NO: 649) |
| RSN-0068 | GSAPGTPGGYAELRMSGGAATSGSETP | RSC-0068 | GTAEAASASGGTPGGYAELRMSGGAPG |
| | GT (SEQ ID NO: 402) | | SP (SEQ ID NO: 650) |
| RSN-0069 | GSAPGASGGYAELRMNGEIATSGSETP | RSC-0069 | GTAEAASASGGASGGYAELRMNGEIPG |
| | GT (SEQ ID NO: 403) | | SP (SEQ ID NO: 651) |
| RSN-0070 | GSAPGGTGGYAELRMQGPAATSGSETP | RSC-0070 | GTAEAASASGGGTGGYAELRMQGPAPG |
| | GT (SEQ ID NO: 404) | | SP (SEQ ID NO: 652) |
| RSN-0071 | GSAPGANHTPAGLTGPGARATSGSETP | RSC-0071 | GTAEAASASGGANHTPAGLTGPGARPG |
| | GT (SEQ ID NO: 405) | | SP (SEQ ID NO: 653) |
| RSN-0072 | GSAPGANTAPEGLTGPSTRATSGSETP | RSC-0072 | GTAEAASASGGANTAPEGLTGPSTRPG |
| | GT (SEQ ID NO: 406) | | SP (SEQ ID NO: 654) |
| RSN-0073 | GSAPGTGAPPGGLTGPGTRATSGSETP | RSC-0073 | GTAEAASASGGTGAPPGGLTGPGTRPG |
| | GT (SEQ ID NO: 407) | | SP (SEQ ID NO: 655) |
| RSN-0074 | GSAPGANHEPSGLTEGSPRATSGSETP | RSC-0074 | GTAEAASASGGANHEPSGLTEGSPRPG |
| | GT (SEQ ID NO: 408) | | SP (SEQ ID NO: 656) |
| RSN-0075 | GSAPGANTEPPELGAGTERATSGSETP | RSC-0075 | GTAEAASASGGANTEPPELGAGTERPG |
| | GT (SEQ ID NO: 409) | | SP (SEQ ID NO: 657) |
| RSN-0076 | GSAPGASGPPPGLTGPPGRATSGSETP | RSC-0076 | GTAEAASASGGASGPPPGLTGPPGRPG |
| | GT (SEQ ID NO: 410) | | SP (SEQ ID NO: 658) |
| RSN-0077 | GSAPGASGTPAPLGGEPGRATSGSETP | RSC-0077 | GTAEAASASGGASGTPAPLGGEPGRPG |
| | GT (SEQ ID NO: 411) | | SP (SEQ ID NO: 659) |
| RSN-0078 | GSAPGPAGPPEGLETEAGRATSGSETP | RSC-0078 | GTAEAASASGGPAGPPEGLETEAGRPG |
| | GT (SEQ ID NO: 412) | | SP (SEQ ID NO: 660) |
| RSN-0079 | GSAPGPTSGQGGLTGPESRATSGSETP | RSC-0079 | GTAEAASASGGPTSGQGGLTGPESRPG |
| | GT (SEQ ID NO: 413) | | SP (SEQ ID NO: 661) |
| RSN-0080 | GSAPGSAGGAANLVRGGAIATSGSETP | RSC-0080 | GTAEAASASGGSAGGAANLVRGGAIPG |
| | GT (SEQ ID NO: 414) | | SP (SEQ ID NO: 662) |
| RSN-0081 | GSAPGTGGGAAPLVRGGGAATSGSETP | RSC-0081 | GTAEAASASGGTGGGAAPLVRGGGAPG |
| | GT (SEQ ID NO: 415) | | SP (SEQ ID NO: 663) |
| RSN-0082 | GSAPGAEGGAAALVRGGEIATSGSETP | RSC-0082 | GTAEAASASGGAEGGAAALVRGGEIPG |
| | GT (SEQ ID NO: 416) | | SP (SEQ ID NO: 664) |
| RSN-0083 | GSAPGGPGGAALLVRGGPAATSGSETP | RSC-0083 | GTAEAASASGGGPGGAALLVRGGPAPG |
| | GT (SEQ ID NO: 417) | | SP (SEQ ID NO: 665) |
| RSN-0084 | GSAPGEAGGAAFLVRGGSIATSGSETP | RSC-0084 | GTAEAASASGGEAGGAAFLVRGGSIPG |
| | GT (SEQ ID NO: 418) | | SP (SEQ ID NO: 666) |
| RSN-0085 | GSAPGPGGGAASLVRGGTAATSGSETP | RSC-0085 | GTAEAASASGGPGGGAASLVRGGTAPG |
| | GT (SEQ ID NO: 419) | | SP (SEQ ID NO: 667) |
| RSN-0086 | GSAPGSEGGAATLVRGGAIATSGSETP | RSC-0086 | GTAEAASASGGSEGGAATLVRGGAIPG |
| | GT (SEQ ID NO: 420) | | SP (SEQ ID NO: 668) |
| RSN-0087 | GSAPGTPGGAAGLVRGGGAATSGSETP | RSC-0087 | GTAEAASASGGTPGGAAGLVRGGGAPG |
| | GT (SEQ ID NO: 421) | | SP (SEQ ID NO: 669) |
| RSN-0088 | GSAPGASGGAADLVRGGEIATSGSETP | RSC-0088 | GTAEAASASGGASGGAADLVRGGEIPG |
| | GT (SEQ ID NO: 422) | | SP(SEQ ID NO: 670) |
| RSN-0089 | GSAPGGTGGAGNLVRGGPAATSGSETP | RSC-0089 | GTAEAASASGGGTGGAGNLVRGGPAPG |
| | GT (SEQ ID NO: 423) | | SP (SEQ ID NO: 671) |
| RSN-0090 | GSAPGEAGGAPNLVRGGSIATSGSETP | RSC-0090 | GTAEAASASGGEAGGAPNLVRGGSIPG |
| | GT (SEQ ID NO: 424) | | SP (SEQ ID NO: 672) |
| RSN-0091 | GSAPGPGGGAVNLVRGGTAATSGSETP | RSC-0091 | GTAEAASASGGPGGGAVNLVRGGTAPG |
| | GT (SEQ ID NO: 425) | | SP (SEQ ID NO: 673) |
| RSN-0092 | GSAPGSEGGALNLVRGGAIATSGSETP | RSC-0092 | GTAEAASASGGSEGGALNLVRGGAIPG |
| | GT (SEQ ID NO: 426) | | SP (SEQ ID NO: 674) |
| RSN-0093 | GSAPGTPGGASNLVRGGGAATSGSETP | RSC-0093 | GTAEAASASGGTPGGASNLVRGGGAPG |
| | GT (SEQ ID NO: 427) | | SP (SEQ ID NO: 675) |
| RSN-0094 | GSAPGASGGATNLVRGGEIATSGSETP | RSC-0094 | GTAEAASASGGASGGATNLVRGGEIPG |
| | GT (SEQ ID NO: 428) | | SP (SEQ ID NO: 676) |
| RSN-0095 | GSAPGGTGGAQNLVRGGPAATSGSETP | RSC-0095 | GTAEAASASGGGTGGAQNLVRGGPAPG |
| | GT (SEQ ID NO: 429) | | SP(SEQ ID NO: 677) |
| RSN-0096 | GSAPGEAGGAENLVRGGSIATSGSETP | RSC-0096 | GTAEAASASGGEAGGAENLVRGGSIPG |
| | GT (SEQ ID NO: 430) | | SP (SEQ ID NO: 678) |
| RSN-1517 | GSAPEAGRSANHEPLGLVATATSGSET | RSC-1517 | GTAEAASASGEAGRSANHEPLGLVATP |
| | PGT (SEQ ID NO: 431) | | GSP (SEQ ID NO: 679) |
| BSRS-A1-2 | GSAPASGRSTNAGPSGLAGPATSGSET | BSRS-A1-3 | GTAEAASASGASGRSTNAGPSGLAGPP |
| | PGT (SEQ ID NO: 432) | | GSP (SEQ ID NO: 680) |
| BSRS-A2-2 | GSAPASGRSTNAGPQGLAGQATSGSET | BSRS-A2-3 | GTAEAASASGASGRSTNAGPQGLAGQP |
| | PGT (SEQ ID NO: 433) | | GSP (SEQ ID NO: 681) |
| BSRS-A3-3 | GSAPASGRSTNAGPPGLTGPATSGSET | BSRS-A3-3 | GTAEAASASGASGRSTNAGPPGLTGPP |
| | PGT (SEQ ID NO: 434) | | GSP (SEQ ID NO: 682) |
| VP-1 | GSAPASSRGTNAGPAGLTGPATSGSET | VP-1 | GTAEAASASGASSRGTNAGPAGLTGPP |
| | PGT (SEQ ID NO: 435) | | GSP (SEQ ID NO: 683) |
| RSN-1752 | GSAPASSRTTNTGPSTLTGPATSGSET | RSC-1752 | GTAEAASASGASSRTTNTGPSTLTGPP |
| | PGT (SEQ ID NO: 436) | | GSP (SEQ ID NO: 684) |
| RSN-1512 | GSAPAAGRSDNGTPLELVAPATSGSET | RSC-1512 | GTAEAASASGAAGRSDNGTPLELVAPP |
| | PGT (SEQ ID NO: 437) | | GSP (SEQ ID NO: 685) |
| RSN-1517 | GSAPEAGRSANHEPLGLVATATSGSET | RSC-1517 | GTAEAASASGEAGRSANHEPLGLVATP |
| | PGT (SEQ ID NO: 438) | | GSP (SEQ ID NO: 686) |
| VP-2 | GSAPASGRGTNAGPAGLTGPATSGSET | VP-2 | GTAEAASASGASGRGTNAGPAGLTGPP |
| | PGT (SEQ ID NO: 439) | | GSP (SEQ ID NO: 687) |
| RSN-1018 | GSAPLFGRNDNHEPLELGGGATSGSET | RSC-1018 | GTAEAASASGLFGRNDNHEPLELGGGP |
| | PGT (SEQ ID NO: 440) | | GSP (SEQ ID NO: 688) |
| RSN-1053 | GSAPTAGRSDNLEPLGLVFGATSGSET | RSC-1053 | GTAEAASASGTAGRSDNLEPLGLVFGP |
| | PGT (SEQ ID NO: 441) | | GSP (SEQ ID NO: 689) |
| RSN-1059 | GSAPLDGRSDNFHPPELVAGATSGSET | RSC-1059 | GTAEAASASGLDGRSDNFHPPELVAGP |
| | PGT (SEQ ID NO: 442) | | GSP (SEQ ID NO: 690) |
| RSN-1065 | GSAPLEGRSDNEEPENLVAGATSGSET | RSC-1065 | GTAEAASASGLEGRSDNEEPENLVAGP |
| | PGT (SEQ ID NO: 443) | | GSP (SEQ ID NO: 691) |
| RSN-1167 | GSAPLKGRSDNNAPLALVAGATSGSET | RSC-1167 | GTAEAASASGLKGRSDNNAPLALVAGP |
| | PGT (SEQ ID NO: 444) | | GSP (SEQ ID NO: 692) |
| RSN-1201 | GSAPVYSRGTNAGPHGLTGRATSGSET | RSC-1201 | GTAEAASASGVYSRGTNAGPHGLTGRP |
| | PGT (SEQ ID NO: 445) | | GSP (SEQ ID NO: 693) |
| RSN-1218 | GSAPANSRGTNKGFAGLIGPATSGSET | RSC-1218 | GTAEAASASGANSRGTNKGFAGLIGPP |
| | PGT (SEQ ID NO: 446) | | GSP (SEQ ID NO: 694) |
| RSN-1226 | GSAPASSRLTNEAPAGLTIPATSGSET | RSC-1226 | GTAEAASASGASSRLTNEAPAGLTIPP |
| | PGT (SEQ ID NO: 447) | | GSP (SEQ ID NO: 695) |
| RSN-1254 | GSAPDQSRGTNAGPEGLTDPATSGSET | RSC-1254 | GTAEAASASGDQSRGTNAGPEGLTDPP |
| | PGT (SEQ ID NO: 448) | | GSP (SEQ ID NO: 696) |
| RSN-1256 | GSAPESSRGTNIGQGGLTGPATSGSET | RSC-1256 | GTAEAASASGESSRGTNIGQGGLTGPP |
| | PGT (SEQ ID NO: 449) | | GSP (SEQ ID NO: 697) |
| RSN-1261 | GSAPSSSRGTNQDPAGLTIPATSGSET | RSC-1261 | GTAEAASASGSSSRGTNQDPAGLTIPP |
| | PGT (SEQ ID NO: 450) | | GSP (SEQ ID NO: 698) |
| RSN-1293 | GSAPASSRGQNHSPMGLTGPATSGSET | RSC-1293 | GTAEAASASGASSRGQNHSPMGLTGPP |
| | PGT (SEQ ID NO: 451) | | GSP (SEQ ID NO: 699) |
| RSN-1309 | GSAPAYSRGPNAGPAGLEGRATSGSET | RSC-1309 | GTAEAASASGAYSRGPNAGPAGLEGRP |
| | PGT (SEQ ID NO: 452) | | GSP (SEQ ID NO: 700) |
| RSN-1326 | GSAPASERGNNAGPANLTGFATSGSET | RSC-1326 | GTAEAASASGASERGNNAGPANLTGFP |
| | PGT (SEQ ID NO: 453) | | GSP (SEQ ID NO: 701) |
| RSN-1345 | GSAPASHRGTNPKPAILTGPATSGSET | RSC-1345 | GTAEAASASGASHRGTNPKPAILTGPP |
| | PGT (SEQ ID NO: 454) | | GSP (SEQ ID NO: 702) |
| RSN-1354 | GSAPMSSRRTNANPAQLTGPATSGSET | RSC-1354 | GTAEAASASGMSSRRTNANPAQLTGPP |
| | PGT (SEQ ID NO: 455) | | GSP (SEQ ID NO: 703) |
| RSN-1426 | GSAPGAGRTDNHEPLELGAAATSGSET | RSC-1426 | GTAEAASASGGAGRTDNHEPLELGAAP |
| | PGT (SEQ ID NO: 456) | | GSP (SEQ ID NO: 704) |
| RSN-1478 | GSAPLAGRSENTAPLELTAGATSGSET | RSC-1478 | GTAEAASASGLAGRSENTAPLELTAGP |
| | PGT (SEQ ID NO: 457) | | GSP (SEQ ID NO: 705) |
| RSN-1479 | GSAPLEGRPDNHEPLALVASATSGSET | RSC-1479 | GTAEAASASGLEGRPDNHEPLALVASP |
| | PGT (SEQ ID NO: 458) | | GSP (SEQ ID NO: 706) |
| RSN-1496 | GSAPLSGRSDNEEPLALPAGATSGSET | RSC-1496 | GTAEAASASGLSGRSDNEEPLALPAGP |
| | PGT (SEQ ID NO: 459) | | GSP (SEQ ID NO: 707) |
| RSN-1508 | GSAPEAGRTDNHEPLELSAPATSGSET | RSC-1508 | GTAEAASASGEAGRTDNHEPLELSAPP |
| | PGT (SEQ ID NO: 460) | | GSP (SEQ ID NO: 708) |
| RSN-1513 | GSAPEGGRSDNHGPLELVSGATSGSET | RSC-1513 | GTAEAASASGEGGRSDNHGPLELVSGP |
| | PGT (SEQ ID NO: 461) | | GSP (SEQ ID NO: 709) |
| RSN-1516 | GSAPLSGRSDNEAPLELEAGATSGSET | RSC-1516 | GTAEAASASGLSGRSDNEAPLELEAGP |
| | PGT (SEQ ID NO: 462) | | GSP (SEQ ID NO: 710) |
| RSN-1524 | GSAPLGGRADNHEPPELGAGATSGSET | RSC-1524 | GTAEAASASGLGGRADNHEPPELGAGP |
| | PGT (SEQ ID NO: 463) | | GSP (SEQ ID NO: 711) |
| RSN-1622 | GSAPPPSRGTNAEPAGLTGEATSGSET | RSC-1622 | GTAEAASASGPPSRGTNAEPAGLTGEP |
| | PGT (SEQ ID NO: 464) | | GSP (SEQ ID NO: 712) |
| RSN-1629 | GSAPASTRGENAGPAGLEAPATSGSET | RSC-1629 | GTAEAASASGASTRGENAGPAGLEAPP |
| | PGT (SEQ ID NO: 465) | | GSP (SEQ ID NO: 713) |
| RSN-1664 | GSAPESSRGTNGAPEGLTGPATSGSET | RSC-1664 | GTAEAASASGESSRGTNGAPEGLTGPP |
| | PGT (SEQ ID NO: 466) | | GSP (SEQ ID NO: 714) |
| RSN-1667 | GSAPASSRATNESPAGLTGEATSGSET | RSC-1667 | GTAEAASASGASSRATNESPAGLTGEP |
| | PGT (SEQ ID NO: 467) | | GSP (SEQ ID NO: 715) |
| RSN-1709 | GSAPASSRGENPPPGGLTGPATSGSET | RSC-1709 | GTAEAASASGASSRGENPPPGGLTGPP |
| | PGT (SEQ ID NO: 468) | | GSP (SEQ ID NO: 716) |
| RSN-1712 | GSAPAASRGTNTGPAELTGSATSGSET | RSC-1712 | GTAEAASASGAASRGTNTGPAELTGSP |
| | PGT (SEQ ID NO: 469) | | GSP (SEQ ID NO: 717) |
| RSN-1727 | GSAPAGSRTTNAGPGGLEGPATSGSET | RSC-1727 | GTAEAASASGAGSRTTNAGPGGLEGPP |
| | PGT (SEQ ID NO: 470) | | GSP (SEQ ID NO: 718) |
| RSN-1754 | GSAPAPSRGENAGPATLTGAATSGSET | RSC-1754 | GTAEAASASGAPSRGENAGPATLTGAP |
| | PGT (SEQ ID NO: 471) | | GSP (SEQ ID NO: 719) |
| RSN-1819 | GSAPESGRAANTGPPTLTAPATSGSET | RSC-1819 | GTAEAASASGESGRAANTGPPTLTAPP |
| | PGT (SEQ ID NO: 472) | | GSP (SEQ ID NO: 720) |
| RSN-1832 | GSAPNPGRAANEGPPGLPGSATSGSET | RSC-1832 | GTAEAASASGNPGRAANEGPPGLPGSP |
| | PGT (SEQ ID NO: 473) | | GSP (SEQ ID NO: 721) |
| RSN-1855 | GSAPESSRAANLTPPELTGPATSGSET | RSC-1855 | GTAEAASASGESSRAANLTPPELTGPP |
| | PGT (SEQ ID NO: 474) | | GSP (SEQ ID NO: 722) |
| RSN-1911 | GSAPASGRAANETPPGLTGAATSGSET | RSC-1911 | GTAEAASASGASGRAANETPPGLTGAP |
| | PGT (SEQ ID NO: 475) | | GSP (SEQ ID NO: 723) |
| RSN-1929 | GSAPNSGRGENLGAPGLTGTATSGSET | RSC-1929 | GTAEAASASGNSGRGENLGAPGLTGTP |
| | PGT (SEQ ID NO: 476) | | GSP (SEQ ID NO: 724) |
| RSN-1951 | GSAPTTGRAANLTPAGLTGPATSGSET | RSC-1951 | GTAEAASASGTTGRAANLTPAGLTGPP |
| | PGT (SEQ ID NO: 477) | | GSP (SEQ ID NO: 725) |
| RSN-2295 | GSAPEAGRSANHTPAGLTGPATSGSET | RSC-2295 | GTAEAASASGEAGRSANHTPAGLTGPP |
| | PGT (SEQ ID NO: 478) | | GSP (SEQ ID NO: 726) |
| RSN-2298 | GSAPESGRAANTTPAGLTGPATSGSET | RSC-2298 | GTAEAASASGESGRAANTTPAGLTGPP |
| | PGT (SEQ ID NO: 479) | | GSP (SEQ ID NO: 727) |
| RSN-2038 | GSAPTTGRATEAANLTPAGLTGPATSG | RSC-2038 | GTAEAASASGTTGRATEAANLTPAGLT |
| | SETPGT (SEQ ID NO: 480) | | GPPGSP (SEQ ID NO: 728) |
| RSN-2072 | GSAPTTGRAEEAANLTPAGLTGPATSG | RSC-2072 | GTAEAASASGTTGRAEEAANLTPAGLT |
| | SETPGT (SEQ ID NO: 481) | | GPPGSP (SEQ ID NO: 729) |
| RSN-2089 | GSAPTTGRAGEAANLTPAGLTGPATSG | RSC-2089 | GTAEAASASGTTGRAGEAANLTPAGLT |
| | SETPGT (SEQ ID NO: 482) | | GPPGSP (SEQ ID NO: 730) |
| RSN-2302 | GSAPTTGRATEAANATPAGLTGPATSG | RSC-2302 | GTAEAASASGTTGRATEAANATPAGLT |
| | SETPGT (SEQ ID NO: 483) | | GPPGSP (SEQ ID NO: 731) |
| RSN-3047 | GSAPTTGRAGEAEGATSAGATGPATSG | RSC-3047 | GTAEAASASGTTGRAGEAEGATSAGAT |
| | SETPGT (SEQ ID NO: 484) | | GPPGSP (SEQ ID NO: 732) |
| RSN-3052 | GSAPTTGEAGEAANATSAGATGPATSG | RSC-3052 | GTAEAASASGTTGEAGEAANATSAGAT |
| | SETPGT (SEQ ID NO: 485) | | GPPGSP (SEQ ID NO: 733) |
| RSN-3043 | GSAPTTGEAGEAAGLTPAGLTGPATSG | RSC-3043 | GTAEAASASGTTGEAGEAAGLTPAGLT |
| | SETPGT (SEQ ID NO: 486) | | GPPGSP (SEQ ID NO: 734) |
| RSN-3041 | GSAPTTGAAGEAANATPAGLTGPATSG | RSC-3041 | GTAEAASASGTTGAAGEAANATPAGLT |
| | SETPGT (SEQ ID NO: 487) | | GPPGSP (SEQ ID NO: 735) |
| RSN-3044 | GSAPTTGRAGEAAGLTPAGLTGPATSG | RSC-3044 | GTAEAASASGTTGRAGEAAGLTPAGLT |
| | SETPGT (SEQ ID NO: 488) | | GPPGSP (SEQ ID NO: 736) |
| RSN-3057 | GSAPTTGRAGEAANATSAGATGPATSG | RSC-3057 | GTAEAASASGTTGRAGEAANATSAGAT |
| | SETPGT (SEQ ID NO: 489) | | GPPGSP (SEQ ID NO: 737) |
| RSN-3058 | GSAPTTGEAGEAAGATSAGATGPATSG | RSC-3058 | GTAEAASASGTTGEAGEAAGATSAGAT |
| | SETPGT (SEQ ID NO: 490) | | GPPGSP (SEQ ID NO: 738) |
| RSN-2485 | GSAPESGRAANTEPPELGAGATSGSET | RSC-2485 | GTAEAASASGESGRAANTEPPELGAGP |
| | PGT (SEQ ID NO: 491) | | GSP (SEQ ID NO: 739) |
| RSN-2486 | GSAPESGRAANTAPEGLTGPATSGSET | RSC-2486 | GTAEAASASGESGRAANTAPEGLTGPP |
| | PGT (SEQ ID NO: 492) | | GSP (SEQ ID NO: 740) |
| RSN-2488 | GSAPEPGRAANHEPSGLTEGATSGSET | RSC-2488 | GTAEAASASGEPGRAANHEPSGLTEGP |
| | PGT (SEQ ID NO: 493) | | GSP (SEQ ID NO: 741) |
| RSN-2599 | GSAPESGRAANHTGAPPGGLTGPATSG | RSC-2599 | GTAEAASASGESGRAANHTGAPPGGLT |
| | SETPGT (SEQ ID NO: 494) | | GPPGSP (SEQ ID NO: 742) |
| RSN-2706 | GSAPTTGRTGEGANATPGGLTGPATSG | RSC-2706 | GTAEAASASGTTGRTGEGANATPGGLT |
| | SETPGT (SEQ ID NO: 495) | | GPPGSP (SEQ ID NO: 743) |
| RSN-2707 | GSAPRTGRSGEAANETPEGLEGPATSG | RSC-2707 | GTAEAASASGRTGRSGEAANETPEGLE |
| | SETPGT (SEQ ID NO: 496) | | GPPGSP (SEQ ID NO: 744) |
| RSN-2708 | GSAPRTGRTGESANETPAGLGGPATSG | RSC-2708 | GTAEAASASGRTGRTGESANETPAGLG |
| | SETPGT (SEQ ID NO: 497) | | GPPGSP (SEQ ID NO: 745) |
| RSN-2709 | GSAPSTGRTGEPANETPAGLSGPATSG | RSC-2709 | GTAEAASASGSTGRTGEPANETPAGLS |
| | SETPGT (SEQ ID NO: 498) | | GPPGSP (SEQ ID NO: 746) |
| RSN-2710 | GSAPTTGRAGEPANATPTGLSGPATSG | RSC-2710 | GTAEAASASGTTGRAGEPANATPTGLS |
| | SETPGT (SEQ ID NO: 499) | | GPPGSP (SEQ ID NO: 747) |
| RSN-2711 | GSAPRTGRPGEGANATPTGLPGPATSG | RSC-2711 | GTAEAASASGRTGRPGEGANATPTGLP |
| | SETPGT (SEQ ID NO: 500) | | GPPGSP (SEQ ID NO: 748) |
| RSN-2712 | GSAPRTGRGGEAANATPSGLGGPATSG | RSC-2712 | GTAEAASASGRTGRGGEAANATPSGLG |
| | SETPGT (SEQ ID NO: 501) | | GPPGSP (SEQ ID NO: 749) |
| RSN-2713 | GSAPSTGRSGESANATPGGLGGPATSG | RSC-2713 | GTAEAASASGSTGRSGESANATPGGLG |
| | SETPGT (SEQ ID NO: 502) | | GPPGSP (SEQ ID NO: 750) |
| RSN-2714 | GSAPRTGRTGEEANATPAGLPGPATSG | RSC-2714 | GTAEAASASGRTGRTGEEANATPAGLP |
| | SETPGT (SEQ ID NO: 503) | | GPPGSP (SEQ ID NO: 751) |
| RSN-2715 | GSAPATGRPGEPANTTPEGLEGPATSG | RSC-2715 | GTAEAASASGATGRPGEPANTTPEGLE |
| | SETPGT (SEQ ID NO: 504) | | GPPGSP (SEQ ID NO: 752) |
| RSN-2716 | GSAPSTGRSGEPANATPGGLTGPATSG | RSC-2716 | GTAEAASASGSTGRSGEPANATPGGLT |
| | SETPGT (SEQ ID NO: 505) | | GPPGSP (SEQ ID NO: 753) |
| RSN-2717 | GSAPPTGRGGEGANTTPTGLPGPATSG | RSC-2717 | GTAEAASASGPTGRGGEGANTTPTGLP |
| | SETPGT (SEQ ID NO: 506) | | GPPGSP (SEQ ID NO: 754) |
| RSN-2718 | GSAPPTGRSGEGANATPSGLTGPATSG | RSC-2718 | GTAEAASASGPTGRSGEGANATPSGLT |
| | SETPGT (SEQ ID NO: 507) | | GPPGSP (SEQ ID NO: 755) |
| RSN-2719 | GSAPTTGRASEGANSTPAPLTEPATSG | RSC-2719 | GTAEAASASGTTGRASEGANSTPAPLT |
| | SETPGT (SEQ ID NO: 508) | | EPPGSP (SEQ ID NO: 756) |
| RSN-2720 | GSAPTYGRAAEAANTTPAGLTAPATSG | RSC-2720 | GTAEAASASGTYGRAAEAANTTPAGLT |
| | SETPGT (SEQ ID NO: 509) | | APPGSP (SEQ ID NO: 757) |
| RSN-2721 | GSAPTTGRATEGANATPAELTEPATSG | RSC-2721 | GTAEAASASGTTGRATEGANATPAELT |
| | SETPGT (SEQ ID NO: 510) | | EPPGSP (SEQ ID NO: 758) |
| RSN-2722 | GSAPTVGRASEEANTTPASLTGPATSG | RSC-2722 | GTAEAASASGTVGRASEEANTTPASLT |
| | SETPGT (SEQ ID NO: 511) | | GPPGSP (SEQ ID NO: 759) |
| RSN-2723 | GSAPTTGRAPEAANATPAPLTGPATSG | RSC-2723 | GTAEAASASGTTGRAPEAANATPAPLT |
| | SETPGT (SEQ ID NO: 512) | | GPPGSP (SEQ ID NO: 760) |
| RSN-2724 | GSAPTWGRATEPANATPAPLTSPATSG | RSC-2724 | GTAEAASASGTWGRATEPANATPAPLT |
| | SETPGT (SEQ ID NO: 513) | | SPPGSP (SEQ ID NO: 761) |
| RSN-2725 | GSAPTVGRASESANATPAELTSPATSG | RSC-2725 | GTAEAASASGTVGRASESANATPAELT |
| | SETPGT (SEQ ID NO: 514) | | SPPGSP (SEQ ID NO: 762) |
| RSN-2726 | GSAPTVGRAPEGANSTPAGLTGPATSG | RSC-2726 | GTAEAASASGTVGRAPEGANSTPAGLT |
| | SETPGT (SEQ ID NO: 515) | | GPPGSP (SEQ ID NO: 763) |
| RSN-2727 | GSAPTWGRATEAPNLEPATLTTPATSG | RSC-2727 | GTAEAASASGTWGRATEAPNLEPATLT |
| | SETPGT (SEQ ID NO: 516) | | TPPGSP (SEQ ID NO: 764) |
| RSN-2728 | GSAPTTGRATEAPNLTPAPLTEPATSG | RSC-2728 | GTAEAASASGTTGRATEAPNLTPAPLT |
| | SETPGT (SEQ ID NO: 517) | | EPPGSP (SEQ ID NO: 765) |
| RSN-2729 | GSAPTOGRATEAPNLSPAALTSPATSG | RSC-2729 | GTAEAASASGTOGRATEAPNLSPAALT |
| | SETPGT (SEQ ID NO: 518) | | SPPGSP (SEQ ID NO: 766) |
| RSN-2730 | GSAPTOGRAAEAPNLTPATLTAPATSG | RSC-2730 | GTAEAASASGTOGRAAEAPNLTPATLT |
| | SETPGT (SEQ ID NO: 519) | | APPGSP (SEQ ID NO: 767) |
| RSN-2731 | GSAPTSGRAPEATNLAPAPLTGPATSG | RSC-2731 | GTAEAASASGTSGRAPEATNLAPAPLT |
| | SETPGT (SEQ ID NO: 520) | | GPPGSP (SEQ ID NO: 768) |
| RSN-2732 | GSAPTOGRAAEAANLTPAGLTEPATSG | RSC-2732 | GTAEAASASGTOGRAAEAANLTPAGLT |
| | SETPGT (SEQ ID NO: 521) | | EPPGSP (SEQ ID NO: 769) |
| RSN-2733 | GSAPTTGRAGSAPNLPPTGLTTPATSG | RSC-2733 | GTAEAASASGTTGRAGSAPNLPPTGLT |
| | SETPGT (SEQ ID NO: 522) | | TPPGSP (SEQ ID NO: 770) |
| RSN-2734 | GSAPTTGRAGGAENLPPEGLTAPATSG | RSC-2734 | GTAEAASASGTTGRAGGAENLPPEGLT |
| | SETPGT (SEQ ID NO: 523) | | APPGSP (SEQ ID NO: 771) |
| RSN-2735 | GSAPTTSRAGTATNLTPEGLTAPATSG | RSC-2735 | GTAEAASASGTTSRAGTATNLTPEGLT |
| | SETPGT (SEQ ID NO: 524) | | APPGSP (SEQ ID NO: 772) |
| RSN-2736 | GSAPTTGRAGTATNLPPSGLTTPATSG | RSC-2736 | GTAEAASASGTTGRAGTATNLPPSGLT |
| | SETPGT (SEQ ID NO: 525) | | TPPGSP (SEQ ID NO: 773) |
| RSN-2737 | GSAPTTARAGEAENLSPSGLTAPATSG | RSC-2737 | GTAEAASASGTTARAGEAENLSPSGLT |
| | SETPGT (SEQ ID NO: 526) | | APPGSP (SEQ ID NO: 774) |
| RSN-2738 | GSAPTTGRAGGAGNLAPGGLTEPATSG | RSC-2738 | GTAEAASASGTTGRAGGAGNLAPGGLT |
| | SETPGT (SEQ ID NO: 527) | | EPPGSP (SEQ ID NO: 775) |
| RSN-2739 | GSAPTTGRAGTATNLPPEGLTGPATSG | RSC-2739 | GTAEAASASGTTGRAGTATNLPPEGLT |
| | SETPGT (SEQ ID NO: 528) | | GPPGSP (SEQ ID NO: 776) |
| RSN-2740 | GSAPTTGRAGGAANLAPTGLTEPATSG | RSC-2740 | GTAEAASASGTTGRAGGAANLAPTGLT |
| | SETPGT (SEQ ID NO: 529) | | EPPGSP (SEQ ID NO: 777) |
| RSN-2741 | GSAPTTGRAGTAENLAPSGLTTPATSG | RSC-2741 | GTAEAASASGTTGRAGTAENLAPSGLT |
| | SETPGT (SEQ ID NO: 530) | | TPPGSP (SEQ ID NO: 778) |
| RSN-2742 | GSAPTTGRAGSATNLGPGGLTGPATSG | RSC-2742 | GTAEAASASGTTGRAGSATNLGPGGLT |
| | SETPGT (SEQ ID NO: 531) | | GPPGSP (SEQ ID NO: 779) |
| RSN-2743 | GSAPTTARAGGAENLTPAGLTEPATSG | RSC-2743 | GTAEAASASGTTARAGGAENLTPAGLT |
| | SETPGT (SEQ ID NO: 532) | | EPPGSP (SEQ ID NO: 780) |
| RSN-2744 | GSAPTTARAGSAENLSPSGLTGPATSG | RSC-2744 | GTAEAASASGTTARAGSAENLSPSGLT |
| | SETPGT (SEQ ID NO: 533) | | GPPGSP (SEQ ID NO: 781) |
| RSN-2745 | GSAPTTARAGGAGNLAPEGLTTPATSG | RSC-2745 | GTAEAASASGTTARAGGAGNLAPEGLT |
| | SETPGT (SEQ ID NO: 534) | | TPPGSP (SEQ ID NO: 782) |
| RSN-2746 | GSAPTTSRAGAAENLTPTGLTGPATSG | RSC-2746 | GTAEAASASGTTSRAGAAENLTPTGLT |
| | SETPGT (SEQ ID NO: 535) | | GPPGSP (SEQ ID NO: 783) |
| RSN-2747 | GSAPTYGRTTTPGNEPPASLEAEATSG | RSC-2747 | GTAEAASASGTYGRTTTPGNEPPASLE |
| | SETPGT (SEQ ID NO: 536) | | AEPGSP (SEQ ID NO: 784) |
| RSN-2748 | GSAPTYSRGESGPNEPPPGLTGPATSG | RSC-2748 | GTAEAASASGTYSRGESGPNEPPPGLT |
| | SETPGT (SEQ ID NO: 537) | | GPPGSP (SEQ ID NO: 785) |
| RSN-2749 | GSAPAWGRTGASENETPAPLGGEATSG | RSC-2749 | GTAEAASASGAWGRTGASENETPAPLG |
| | SETPGT (SEQ ID NO: 538) | | GEPGSP (SEQ ID NO: 786) |
| RSN-2750 | GSAPRWGRAETTPNTPPEGLETEATSG | RSC-2750 | GTAEAASASGRWGRAETTPNTPPEGLE |
| | SETPGT (SEQ ID NO: 539) | | TEPGSP (SEQ ID NO: 787) |
| RSN-2751 | GSAPESGRAANHTGAEPPELGAGATSG | RSC-2751 | GTAEAASASGESGRAANHTGAEPPELG |
| | SETPGT (SEQ ID NO: 540) | | AGPGSP (SEQ ID NO: 788) |
| RSN-2754 | GSAPTTGRAGEAANLTPAGLTESATSG | RSC-2754 | GTAEAASASGTTGRAGEAANLTPAGLT |
| | SETPGT (SEQ ID NO: 541) | | ESPGSP (SEQ ID NO: 789) |
| RSN-2755 | GSAPTTGRAGEAANLTPAALTESATSG | RSC-2755 | GTAEAASASGTTGRAGEAANLTPAALT |
| | SETPGT (SEQ ID NO: 542) | | ESPGSP (SEQ ID NO: 790) |
| RSN-2756 | GSAPTTGRAGEAANLTPAPLTESATSG | RSC-2756 | GTAEAASASGTTGRAGEAANLTPAPLT |
| | SETPGT (SEQ ID NO: 543) | | ESPGSP (SEQ ID NO: 791) |
| RSN-2757 | GSAPTTGRAGEAANLTPEPLTESATSG | RSC-2757 | GTAEAASASGTTGRAGEAANLTPEPLT |
| | SETPGT (SEQ ID NO: 544) | | ESPGSP (SEQ ID NO: 792) |
| RSN-2758 | GSAPTTGRAGEAANLTPAGLTGAATSG | RSC-2758 | GTAEAASASGTTGRAGEAANLTPAGLT |
| | SETPGT (SEQ ID NO: 545) | | GAPGSP (SEQ ID NO: 793) |
| RSN-2759 | GSAPTTGRAGEAANLTPEGLTGAATSG | RSC-2759 | GTAEAASASGTTGRAGEAANLTPEGLT |
| | SETPGT (SEQ ID NO: 546) | | GAPGSP (SEQ ID NO: 794) |
| RSN-2760 | GSAPTTGRAGEAANLTPEPLTGAATSG | RSC-2760 | GTAEAASASGTTGRAGEAANLTPEPLT |
| | SETPGT (SEQ ID NO: 547) | | GAPGSP (SEQ ID NO: 795) |
| RSN-2761 | GSAPTTGRAGEAANLTPAGLTEAATSG | RSC-2761 | GTAEAASASGTTGRAGEAANLTPAGLT |
| | SETPGT (SEQ ID NO: 548) | | EAPGSP (SEQ ID NO: 796) |
| RSN-2762 | GSAPTTGRAGEAANLTPEGLTEAATSG | RSC-2762 | GTAEAASASGTTGRAGEAANLTPEGLT |
| | SETPGT (SEQ ID NO: 549) | | EAPGSP (SEQ ID NO: 797) |
| RSN-2763 | GSAPTTGRAGEAANLTPAPLTEAATSG | RSC-2763 | GTAEAASASGTTGRAGEAANLTPAPLT |
| | SETPGT (SEQ ID NO: 550) | | EAPGSP (SEQ ID NO: 798) |
| RSN-2764 | GSAPTTGRAGEAANLTPEPLTEAATSG | RSC-2764 | GTAEAASASGTTGRAGEAANLTPEPLT |
| | SETPGT (SEQ ID NO: 551) | | EAPGSP (SEQ ID NO: 799) |
| RSN-2765 | GSAPTTGRAGEAANLTPEPLTGPATSG | RSC-2765 | GTAEAASASGTTGRAGEAANLTPEPLT |
| | SETPGT (SEQ ID NO: 552) | | GPPGSP (SEQ ID NO: 800) |
| RSN-2766 | GSAPTTGRAGEAANLTPAGLTGGATSG | RSC-2766 | GTAEAASASGTTGRAGEAANLTPAGLT |
| | SETPGT (SEQ ID NO: 553) | | GGPGSP (SEQ ID NO: 801) |
| RSN-2767 | GSAPTTGRAGEAANLTPEGLTGGATSG | RSC-2767 | GTAEAASASGTTGRAGEAANLTPEGLT |
| | SETPGT (SEQ ID NO: 554) | | GGPGSP (SEQ ID NO: 802) |
| RSN-2768 | GSAPTTGRAGEAANLTPEALTGGATSG | RSC-2768 | GTAEAASASGTTGRAGEAANLTPEALT |
| | SETPGT (SEQ ID NO: 555) | | GGPGSP (SEQ ID NO: 803) |
| RSN-2769 | GSAPTTGRAGEAANLTPEPLTGGATSG | RSC-2769 | GTAEAASASGTTGRAGEAANLTPEPLT |
| | SETPGT (SEQ ID NO: 556) | | GGPGSP (SEQ ID NO: 804) |
| RSN-2770 | GSAPTTGRAGEAANLTPAGLTEGATSG | RSC-2770 | GTAEAASASGTTGRAGEAANLTPAGLT |
| | SETPGT (SEQ ID NO: 557) | | EGPGSP (SEQ ID NO: 805) |
| RSN-2771 | GSAPTTGRAGEAANLTPEGLTEGATSG | RSC-2771 | GTAEAASASGTTGRAGEAANLTPEGLT |
| | SETPGT (SEQ ID NO: 558) | | EGPGSP (SEQ ID NO: 806) |
| RSN-2772 | GSAPTTGRAGEAANLTPAPLTEGATSG | RSC-2772 | GTAEAASASGTTGRAGEAANLTPAPLT |
| | SETPGT (SEQ ID NO: 559) | | EGPGSP (SEQ ID NO: 807) |
| RSN-2773 | GSAPTTGRAGEAANLTPEPLTEGATSG | RSC-2773 | GTAEAASASGTTGRAGEAANLTPEPLT |
| | SETPGT (SEQ ID NO: 560) | | EGPGSP (SEQ ID NO: 808) |
| RSN-3047 | GSAPTTGRAGEAEGATSAGATGPATSG | RSC-3047 | GTAEAASASGTTGRAGEAEGATSAGAT |
| | SETPGT (SEQ ID NO: 561) | | GPPGSP (SEQ ID NO: 809) |
| RSN-2783 | GSAPEAGRSAEATSAGATGPATSGSET | RSC-2783 | GTAEAASASGEAGRSAEATSAGATGPP |
| | PGT (SEQ ID NO: 562) | | GSP (SEQ ID NO: 810) |
| RSN-3107 | GSAPSASGTYSRGESGPGSPATSGSET | RSC-3107 | GTAEAASASGSASGTYSRGESGPGSPP |
| | PGT (SEQ ID NO: 563) | | GSP (SEQ ID NO: 811) |
| RSN-3103 | GSAPSASGEAGRTDTHPGSPATSGSET | RSC-3103 | GTAEAASASGSASGEAGRTDTHPGSPP |
| | PGT (SEQ ID NO: 564) | | GSP (SEQ ID NO: 812) |
| RSN-3102 | GSAPSASGEPGRAAEHPGSPATSGSET | RSC-3102 | GTAEAASASGSASGEPGRAAEHPGSPP |
| | PGT (SEQ ID NO: 565) | | GSP (SEQ ID NO: 813) |
| RSN-3119 | GSAPSPAGESSRGTTIAGSPATSGSET | RSC-3119 | GTAEAASASGSPAGESSRGTTIAGSPP |
| | PGT (SEQ ID NO: 566) | | GSP (SEQ ID NO: 814) |
| RSN-3043 | GSAPTTGEAGEAAGLTPAGLTGPATSG | RSC-3043 | GTAEAASASGTTGEAGEAAGLTPAGLT |
| | SETPGT (SEQ ID NO: 567) | | GPPGSP (SEQ ID NO: 815) |
| RSN-2789 | GSAPEAGESAGATPAGLTGPATSGSET | RSC-2789 | GTAEAASASGEAGESAGATPAGLTGPP |
| | PGT (SEQ ID NO: 568) | | GSP (SEQ ID NO: 816) |
| RSN-3109 | GSAPSASGAPLELEAGPGSPATSGSET | RSC-3109 | GTAEAASASGSASGAPLELEAGPGSPP |
| | PGT (SEQ ID NO: 569) | | GSP (SEQ ID NO: 817) |
| RSN-3110 | GSAPSASGEPPELGAGPGSPATSGSET | RSC-3110 | GTAEAASASGSASGEPPELGAGPGSPP |
| | PGT (SEQ ID NO: 570) | | GSP (SEQ ID NO: 818) |
| RSN-3111 | GSAPSASGEPSGLTEGPGSPATSGSET | RSC-3111 | GTAEAASASGSASGEPSGLTEGPGSPP |
| | PGT (SEQ ID NO: 571) | | GSP (SEQ ID NO: 819) |
| RSN-3112 | GSAPSASGTPAPLTEPPGSPATSGSET | RSC-3112 | GTAEAASASGSASGTPAPLTEPPGSPP |
| | PGT (SEQ ID NO: 572) | | GSP (SEQ ID NO: 820) |
| RSN-3113 | GSAPSASGTPAELTEPPGSPATSGSET | RSC-3113 | GTAEAASASGSASGTPAELTEPPGSPP |
| | PGT (SEQ ID NO: 573) | | GSP (SEQ ID NO: 821) |
| RSN-3114 | GSAPSASGPPPGLTGPPGSPATSGSET | RSC-3114 | GTAEAASASGSASGPPPGLTGPPGSPP |
| | PGT (SEQ ID NO: 574) | | GSP (SEQ ID NO: 822) |
| RSN-3115 | GSAPSASGTPAPLGGEPGSPATSGSET | RSC-3115 | GTAEAASASGSASGTPAPLGGEPGSPP |
| | PGT (SEQ ID NO: 575) | | GSP (SEQ ID NO: 823) |
| RSN-3125 | GSAPSPAGAPEGLTGPAGSPATSGSET | RSC-3125 | GTAEAASASGSPAGAPEGLTGPAGSPP |
| | PGT (SEQ ID NO: 576) | | GSP (SEQ ID NO: 824) |
| RSN-3126 | GSAPSPAGPPEGLETEAGSPATSGSET | RSC-3126 | GTAEAASASGSPAGPPEGLETEAGSPP |
| | PGT (SEQ ID NO: 577) | | GSP (SEQ ID NO: 825) |
| RSN-3127 | GSAPSPTSGQGGLTGPGSEPATSGSET | RSC-3127 | GTAEAASASGSPTSGQGGLTGPGSEPP |
| | PGT (SEQ ID NO: 578) | | GSP (SEQ ID NO: 826) |
| RSN-3131 | GSAPSESAPPEGLETESTEPATSGSET | RSC-3131 | GTAEAASASGSESAPPEGLETESTEPP |
| | PGT (SEQ ID NO: 579) | | GSP (SEQ ID NO: 827) |
| RSN-3132 | GSAPSEGSEPLELGAASETPATSGSET | RSC-3132 | GTAEAASASGSEGSEPLELGAASETPP |
| | PGT (SEQ ID NO: 580) | | GSP (SEQ ID NO: 828) |
| RSN-3133 | GSAPSEGSGPAGLEAPSETPATSGSET | RSC-3133 | GTAEAASASGSEGSGPAGLEAPSETPP |
| | PGT (SEQ ID NO: 581) | | GSP (SEQ ID NO: 829) |
| RSN-3138 | GSAPSEPTPPASLEAEPGSPATSGSET | RSC-3138 | GTAEAASASGSEPTPPASLEAEPGSPP |
| | PGT (SEQ ID NO: 582) | | GSP (SEQ ID NO: 830) |

The RS of the disclosure are useful for inclusion in recombinant polypeptides as therapeutics for treatment of cancers, autoimmune diseases, inflammatory diseases and other conditions where localized activation of the recombinant polypeptide is desirable. The subject compositions address an unmet need and are superior in one or more aspects including enhanced terminal half-life, targeted delivery, and improved therapeutic ratio with reduced toxicity to healthy tissues compared to conventional antibody therapeutics or bispecific antibody therapeutics that are active upon injection.

In one embodiment, a BP incorporated into a BPXTEN fusion protein can have a sequence that exhibits at least about 80% sequence identity to a sequence from Table 3 or Table A, alternatively at least about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%, or about 100% sequence identity as compared with a sequence from Table 3 or Table A. The BP of the foregoing embodiment can be evaluated for activity using assays or measured or determined parameters as described herein, and those sequences that retain at least about 40%, or about 50%, or about 55%, or about 60%, or about 70%, or about 80%, or about 90%, or about 95% or more activity compared to the corresponding native BP sequence would be considered suitable for inclusion in the subject BPXTEN. The BP found to retain a suitable level of activity can be linked to one or more XTEN polypeptides described hereinabove. In one embodiment, a BP found to retain a suitable level of activity can be linked to one or more XTEN polypeptides having at least about 80% sequence identity to a sequence from Tables 2a-2b, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared with a sequence of Tables 2a-2b, resulting in a chimeric fusion protein.

The disclosure contemplates substitution of other BP selected from Table 3 or Table A linked to one or two XTEN, which may be the same or different, selected from Tables 2a-2b. In the foregoing fusion proteins hereinabove described in this paragraph, the BPXTEN fusion protein can further comprise a cleavage sequence from Table 5; the cleavage sequence being located between the BP and the XTEN or between adjacent BP. In some cases, the BPXTEN comprising the cleavage sequences will also have one or more spacer sequence amino acids between the BP and the cleavage sequence or the XTEN and the cleavage sequence to facilitate access of the protease; the spacer amino acids comprising any natural amino acid, including glycine and alanine as preferred amino acids.

### Targeting Moieties

In certain embodiments, it is contemplated that the XPACs of the present invention also may further comprise a tumor targeting moiety that allows the XPAC to bind to an antigen expressed on the tumor. This can be achieved by including one further domain in the chimeric polypeptide (XPAC) to influence its movements within the body. For example, the chimeric nucleic acids can encode a domain that directs the polypeptide to a location in the body, e.g., tumor cells or a site of inflammation. Exemplary and suitable targeting moieties domains comprise those that have a cognate ligand that is overexpressed in inflamed tissues, e.g., the IL-1 receptor, or the IL-6 receptor. In other embodiments, the suitable targeting moieties comprise those who have a cognate ligand that is overexpressed in tumor tissue, e.g., Epcam, CEA or mesothelin. In some embodiments, the targeting domain is linked to the cytokine via a linker which is cleaved at the site of action (e.g., by inflammation or cancer specific proteases) releasing the cytokine full activity at the desired site. In some embodiments, the targeting and/or retention domain is linked to the interleukin via a linker which is not cleaved at the site of action (e.g. by inflammation or cancer specific proteases), causing the cytokine to remain at the desired site.

Particularly preferred targeting moieties target antigens expressed on the surface of a diseased cell or tissue, for example a tumor or a cancer cell. Antigens useful for tumor targeting and retention include but are not limited to EpCAM, EGFR, HER-2, HER-3, c-Met, FOLR1, and CEA. Pharmaceutical compositions disclosed herein, also include proteins comprising two targeting and/or retention domains that bind to two different target antigens known to be expressed on a diseased cell or tissue. Exemplary pairs of antigen binding domains include but are not limited to EGFR/CEA, EpCAM/CEA, and HER-2/HER-3.

Suitable targeting moieties include antigen-binding domains, such as antibodies and fragments thereof including, a polyclonal antibody, a recombinant antibody, a human antibody, a humanized antibody a single chain variable fragment (scFv), single-domain antibody such as a heavy γ chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

In some embodiments, the targeting moieties specifically bind to a cell surface molecule. In some embodiments, the targeting and/or retention domains specifically bind to a tumor antigen. In some embodiments, the targeting polypeptides specifically and independently bind to a tumor antigen selected from at least one of Fibroblast activation protein alpha (FAPa), Trophoblast glycoprotein (5T4), Tumor-associated calcium signal transducer 2 (Trop2), Fibronectin EDB (EDB-FN, see US Publication 20200397915), fibronectin EIIIB domain, CGS-2, EpCAM, EGFR, HER-2, HER-3, cMet, CEA, and FOLR1. In some embodiments, the targeting polypeptides specifically and independently bind to two different antigens, wherein at least one of the antigens is a tumor antigen selected from EpCAM, EGFR, HER-2, HER-3, cMet, CEA, and FOLR1.

The targeted antigen can be a tumor antigen expressed on a tumor cell. Tumor antigens are well known in the art and include, for example, EpCAM, EGFR, HER-2, HER-3, c-Met, FOLR1, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Mudl, Muc16, NaPi2b, Nectin-4, P-cadherin, NY-ESO-1, PRLR, PSCA, PTK7, ROR1, SLC44A4, SLTRK5, SLTRK6, STEAPI, TIM1, Trop2, WTi.

The targeted antigen can be an immune checkpoint protein. Examples of immune checkpoint proteins include but are not limited to CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAMI, CD48, CD70, A2AR, CD39, CD73, B7-H3, B7-H4, BTLA, IDO1, ID02, TDO, KIR, LAG-3, TIM-3, or VISTA.

The targeted antigen can be a cell surface molecule such as a protein, lipid or polysaccharide. In some embodiments, such an antigen on a tumor cell, virally infected cell, bacterially infected cell, damaged red blood cell, arterial plaque cell, inflamed or fibrotic tissue cell. Such an antigen can comprise an immune response modulator such as for example, including but not limited to granulocyte-macrophage colony stimulating factor (GM-CSF), macrophage colony stimulating factor (M-CSF), granulocyte colony stimulating factor (G-CSF), interleukin 2 (IL-2), interleukin 3 (IL-3), interleukin 12 (IL-12), interleukin 15 (IL-15), B7-1 (CD80), B7-2 (CD86), GITRL, CD3, or GITR.

### Pharmacokinetic Properties of BPXTEN

The invention provides BPXTEN fusion proteins with enhanced pharmacokinetics compared to the BP not linked to XTEN that, when used at the dose determined for the composition by the methods described herein, can achieve a circulating concentration resulting in a pharmacologic effect, yet stay within the safety range for biologically active component of the composition for an extended period of time compared to a comparable dose of the BP not linked to XTEN. In such cases, the BPXTEN remains within the therapeutic window for the fusion protein composition for the extended period of time. As used herein, a “comparable dose” means a dose with an equivalent moles/kg for the active BP pharmacophore that is administered to a subject in a comparable fashion. It will be understood in the art that a “comparable dosage” of BPXTEN fusion protein would represent a greater weight of agent but would have essentially the same mole-equivalents of BP in the dose of the fusion protein and/or would have the same approximate molar concentration relative to the BP.

The pharmacokinetic properties of a BP that can be enhanced by linking a given XTEN to the BP include terminal half-life, area under the curve (AUC), Cₘₐₓ volume of distribution, and bioavailability.

As described more fully in the Examples pertaining to pharmacokinetic characteristics of fusion proteins comprising XTEN, it was surprisingly discovered that increasing the length of the XTEN sequence could confer a disproportionate increase in the terminal half-life of a fusion protein comprising the XTEN. Accordingly, the invention provides BPXTEN fusion proteins comprising XTEN wherein the XTEN can be selected to provide a targeted half-life for the BPXTEN composition administered to a subject. In some embodiments, the invention provides monomeric fusion proteins comprising XTEN wherein the XTEN is selected to confer an increase in the terminal half-life for the administered BPXTEN, compared to the corresponding BP not linked to the fusion protein, of at least about two-fold longer, or at least about three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold or greater an increase in terminal half-life compared to the BP not linked to the fusion protein. Similarly, the BPXTEN fusion proteins can have an increase in AUC of at least about 50%, or at least about 60%, or at least about 70%, or at least about 80%, or at least about 90%, or at least about 100%, or at least about 150%, or at least about 200%, or at least about 300% increase in AUC compared to the corresponding BP not linked to the fusion protein. The pharmacokinetic parameters of a BPXTEN can be determined by standard methods involving dosing, the taking of blood samples at times intervals, and the assaying of the protein using ELISA, HPLC, radioassay, or other methods known in the art or as described herein, followed by standard calculations of the data to derive the half-life and other PK parameters.

The invention further provides BPXTEN comprising a first and a second BP molecule, optionally separated by a spacer sequence that may further comprise a cleavage sequence, or separated by a second XTEN sequence. In one embodiment, the BP has less activity when linked to the fusion protein compared to a corresponding BP not linked to the fusion protein. In such case, the BPXTEN can be designed such that upon administration to a subject, the BP component is gradually released by cleavage of the cleavage sequence(s), whereupon it regains activity or the ability to bind to its target receptor or ligand. Accordingly, the BPXTEN of the foregoing serves as a prodrug or a circulating depot, resulting in a longer terminal half-life compared to BP not linked to the fusion protein.

As described herein, in exemplary embodiments, the BPXTEN is an XPAC in which the BP is a cytokine. In preferred embodiments, the activity of the cytokine polypeptide in the context of the XPAC is attenuated, and protease cleavage at the desired site of activity, such as in a tumor microenvironment, releases a form of the cytokine from the XPAC that is much more active as a cytokine receptor agonist than the XPAC. For example, the cytokine-receptor activating (agonist) activity of the fusion polypeptide can be at least about 10 times, at least about 50 times, at least about 100 times, at least about 250 times, at least about 500 times, or at least about 1000 times less than the cytokine receptor activating activity of the cytokine polypeptide as a separate molecular entity. The cytokine polypeptide that is part of the XPAC exists as a separate molecular entity when it contains an amino acid that is substantially identical to the cytokine polypeptide and does not substantially include additional amino acids and is not associated (by covalent or non-covalent bonds) with other molecules. If necessary, a cytokine polypeptide as a separate molecular entity may include some additional amino acid sequences, such as a tag or short sequence to aid in expression and/or purification.

In other examples, the cytokine-receptor activating (agonist) activity of the fusion polypeptide is at least about 10 times, at least about 50 times, at least about 100 times, at least about 250 times, at least about 500 times, or about 1000 times less than the cytokine receptor activating activity of the polypeptide that contains the cytokine polypeptide that is produced by cleavage of the protease cleavable linker in the XPAC. In other words, the cytokine receptor activating (agonist) activity of the polypeptide that contains the cytokine polypeptide that is produced by cleavage of the protease cleavable linker in the XPAC is at least about 10 times, at least about 50 times, at least about 100 times, at least about 250 times, at least about 500 times, or at least about 1000 times greater than the cytokine receptor activating activity of the XPAC.

### Pharmacology and Pharmaceutical Properties of BPXTEN

The present invention provides BPXTEN compositions comprising BP covalently linked to XTEN that can have enhanced properties compared to BP not linked to XTEN, as well as methods to enhance the therapeutic and/or biologic activity or effect of the respective two BP components of the compositions. In addition, the invention provides BPXTEN compositions with enhanced properties compared to those art-known fusion proteins containing immunoglobulin polypeptide partners, polypeptides of shorter length and/or polypeptide partners with repetitive sequences. In addition, BPXTEN fusion proteins provide significant advantages over chemical conjugates, such as pegylated constructs, notably the fact that recombinant BPXTEN fusion proteins can be made in bacterial cell expression systems, which can reduce time and cost at both the research and development and manufacturing stages of a product, as well as result in a more homogeneous, defined product with less toxicity for both the product and metabolites of the BPXTEN compared to pegylated conjugates.

As therapeutic agents, the BPXTEN may possess a number of advantages over therapeutics not comprising XTEN including, for example, increased solubility, increased thermal stability, reduced immunogenicity, increased apparent molecular weight, reduced renal clearance, reduced proteolysis, reduced metabolism, enhanced therapeutic efficiency, a lower effective therapeutic dose, increased bioavailability, increased time between dosages to maintain blood levels within the therapeutic window for the BP, a “tailored” rate of absorption, enhanced lyophilization stability, enhanced serum/plasma stability, increased terminal half-life, increased solubility in blood stream, decreased binding by neutralizing antibodies, decreased receptor-mediated clearance, reduced side effects, retention of receptor/ligand binding affinity or receptor/ligand activation, stability to degradation, stability to freeze-thaw, stability to proteases, stability to ubiquitination, ease of administration, compatibility with other pharmaceutical excipients or carriers, persistence in the subject, increased stability in storage (e.g., increased shelf-life), reduced toxicity in an organism or environment and the like. The net effect of the enhanced properties is that the BPXTEN may result in enhanced therapeutic and/or biologic effect when administered to a subject with a metabolic disease or disorder.

In other cases where, where enhancement of the pharmaceutical or physicochemical properties of the BP is desirable, (such as the degree of aqueous solubility or stability), the length and/or the motif family composition of the first and the second XTEN sequences of the first and the second fusion protein may each be selected to confer a different degree of solubility and/or stability on the respective fusion proteins such that the overall pharmaceutical properties of the BPXTEN composition are enhanced. The BPXTEN fusion proteins can be constructed and assayed, using methods described herein, to confirm the physicochemical properties and the XTEN adjusted, as needed, to result in the desired properties. In one embodiment, the XTEN sequence of the BPXTEN is selected such that the fusion protein has an aqueous solubility that is within at least about 25% greater compared to a BP not linked to the fusion protein, or at least about 30%, or at least about 40%, or at least about 50%, or at least about 75%, or at least about 100%, or at least about 200%, or at least about 300%, or at least about 400%, or at least about 500%, or at least about 1000% greater than the corresponding BP not linked to the fusion protein. In the embodiments hereinabove described in this paragraph, the XTEN of the fusion proteins can have at least about 80% sequence identity, or about 90%, or about 91%, or about 92%, or about 93%, or about 94%, or about 95%, or about 96%, or about 97%, or about 98%, or about 99%, to about 100% sequence identity to an XTEN selected from Tables 2a-2b.

In one embodiment, the invention provides BPXTEN compositions that can maintain the BP component within a therapeutic window for a greater period of time compared to comparable dosages of the corresponding BP not linked to XTEN. It will be understood in the art that a “comparable dosage” of BPXTEN fusion protein would represent a greater weight of agent but would have the same approximate mole-equivalents of BP in the dose of the fusion protein and/or would have the same approximate molar concentration relative to the BP.

The invention also provides methods to select the XTEN appropriate for conjugation to provide the desired pharmacokinetic properties that, when matched with the selection of dose, enable increased efficacy of the administered composition by maintaining the circulating concentrations of the BP within the therapeutic window for an enhanced period of time. As used herein, “therapeutic window” means that amount of drug or biologic as a blood or plasma concentration range, that provides efficacy or a desired pharmacologic effect over time for the disease or condition without unacceptable toxicity; the range of the circulating blood concentrations between the minimal amount to achieve any positive therapeutic effect and the maximum amount which results in a response that is the response immediately before toxicity to the subject (at a higher dose or concentration). Additionally, therapeutic window generally encompasses an aspect of time; the maximum and minimum concentration that results in a desired pharmacologic effect over time that does not result in unacceptable toxicity or adverse events. A dosed composition that stays within the therapeutic window for the subject could also be said to be within the “safety range.”

Dose optimization is important for all drugs, especially for those with a narrow therapeutic window. For example, many peptides involved in glucose homeostasis have a narrow therapeutic window. For a BP with a narrow therapeutic window, such as glucagon or a glucagon analog, a standardized single dose for all patients presenting with a variety of symptoms may not always be effective. Since different glucose regulating peptides are often used together in the treatment of diabetic subjects, the potency of each and the interactive effects achieved by combining and dosing them together must also be taken into account. A consideration of these factors is well within the purview of the ordinarily skilled clinician for the purpose of determining the therapeutically or pharmacologically effective amount of the BPXTEN, versus that amount that would result in unacceptable toxicity and place it outside of the safety range.

In many cases, the therapeutic window for the BP components of the subject compositions have been established and are available in published literature or are stated on the drug label for approved products containing the BP. In other cases, the therapeutic window can be established. The methods for establishing the therapeutic window for a given composition are known to those of skill in the art (see, e.g., Goodman & Gilman's The Pharmacological Basis of Therapeutics, 11^{th} Edition, McGraw-Hill (2005)). For example, by using dose-escalation studies in subjects with the target disease or disorder to determine efficacy or a desirable pharmacologic effect, appearance of adverse events, and determination of circulating blood levels, the therapeutic window for a given subject or population of subjects can be determined for a given drug or biologic, or combinations of biologics or drugs. The dose escalation studies can evaluate the activity of a BPXTEN through metabolic studies in a subject or group of subjects that monitor physiological or biochemical parameters, as known in the art or as described herein for one or more parameters associated with the metabolic disease or disorder, or clinical parameters associated with a beneficial outcome for the particular indication, together with observations and/or measured parameters to determine the no effect dose, adverse events, maximum tolerated dose and the like, together with measurement of pharmacokinetic parameters that establish the determined or derived circulating blood levels. The results can then be correlated with the dose administered and the blood concentrations of the therapeutic that are coincident with the foregoing determined parameters or effect levels. By these methods, a range of doses and blood concentrations can be correlated to the minimum effective dose as well as the maximum dose and blood concentration at which a desired effect occurs and above which toxicity occurs, thereby establishing the therapeutic window for the dosed therapeutic. Blood concentrations of the fusion protein (or as measured by the BP component) above the maximum would be considered outside the therapeutic window or safety range. Thus, by the foregoing methods, a Cₘᵢₙ blood level would be established, below which the BPXTEN fusion protein would not have the desired pharmacologic effect, and a Cₘₐₓ blood level would be established that would represent the highest circulating concentration before reaching a concentration that would elicit unacceptable side effects, toxicity or adverse events, placing it outside the safety range for the BPXTEN. With such concentrations established, the frequency of dosing and the dosage can be further refined by measurement of the Cₘₐₓ and Cₘᵢₙ to provide the appropriate dose and dose frequency to keep the fusion protein(s) within the therapeutic window. One of skill in the art can, by the means disclosed herein or by other methods known in the art, confirm that the administered BPXTEN remains in the therapeutic window for the desired interval or requires adjustment in dose or length or sequence of XTEN. Further, the determination of the appropriate dose and dose frequency to keep the BPXTEN within the therapeutic window establishes the therapeutically effective dose regimen; the schedule for administration of multiple consecutive doses using a therapeutically effective dose of the fusion protein to a subject in need thereof resulting in consecutive Cₘₐₓ peaks and/or Cₘᵢₙ troughs that remain within the therapeutic window and results in an improvement in at least one measured parameter relevant for the target disease, disorder or condition. In some cases, the BPXTEN administered at an appropriate dose to a subject may result in blood concentrations of the BPXTEN fusion protein that remains within the therapeutic window for a period at least about two-fold longer compared to the corresponding BP not linked to XTEN and administered at a comparable dose; alternatively at least about three-fold longer; alternatively at least about four-fold longer; alternatively at least about five-fold longer; alternatively at least about six-fold longer; alternatively at least about seven-fold longer; alternatively at least about eight-fold longer; alternatively at least about nine-fold longer or at least about ten-fold longer or greater compared to the corresponding BP not linked to XTEN and administered at a comparable dose. As used herein, an “appropriate dose” means a dose of a drug or biologic that, when administered to a subject, would result in a desirable therapeutic or pharmacologic effect and a blood concentration within the therapeutic window.

In one embodiment, the BPXTEN administered at a therapeutically effective dose regimen results in a gain in time of at least about three-fold longer; alternatively at least about four-fold longer; alternatively at least about five-fold longer; alternatively at least about six-fold longer; alternatively at least about seven-fold longer; alternatively at least about eight-fold longer; alternatively at least about nine-fold longer or at least about ten-fold longer between at least two consecutive Cₘₐₓ peaks and/or Cₘᵢₙ troughs for blood levels of the fusion protein compared to the corresponding biologically active protein of the fusion protein not linked to the fusion protein and administered at a comparable dose regimen to a subject. In another embodiment, the BPXTEN administered at a therapeutically effective dose regimen results in a comparable improvement in one, or two, or three or more measured parameter using less frequent dosing or a lower total dosage in moles of the fusion protein of the pharmaceutical composition compared to the corresponding biologically active protein component(s) not linked to the fusion protein and administered to a subject using a therapeutically effective dose regimen for the BP. The measured parameters may include any of the clinical, biochemical, or physiological parameters disclosed herein, or others known in the art for assessing subjects with glucose- or insulin-related disorders, metabolic diseases or disorders, coagulation or bleeding disorders, or growth hormone-related disorders.

The activity of the BPXTEN compositions of the invention, including functional characteristics or biologic and pharmacologic activity and parameters that result, may be determined by any suitable screening assay known in the art for measuring the desired characteristic. The activity and structure of the BPXTEN polypeptides comprising BP components may be measured by assays described herein, or by methods known in the art to ascertain the degree of solubility, structure and retention of biologic activity. Assays can be conducted that allow determination of binding characteristics of the BPXTEN for BP receptors or a ligand, including binding constant (K_{d}), EC₅₀ values, as well as their half-life of dissociation of the ligand-receptor complex (T_{1/2}). Binding affinity can be measured, for example, by a competition-type binding assay that detects changes in the ability to specifically bind to a receptor or ligand. Additionally, techniques such as flow cytometry or surface plasmon resonance can be used to detect binding events. The assays may comprise soluble receptor molecules, or may determine the binding to cell-expressed receptors. Such assays may include cell-based assays, including assays for proliferation, cell death, apoptosis and cell migration. Other possible assays may determine receptor binding of expressed polypeptides, wherein the assay may comprise soluble receptor molecules, or may determine the binding to cell-expressed receptors. The binding affinity of a BPXTEN for the target receptors or ligands of the corresponding BP can be assayed using binding or competitive binding assays, such as Biacore assays with chip-bound receptors or binding proteins or ELISA assays, as described in U.S. Pat. No. 5,534,617, assays described in the Examples herein, radio-receptor assays, or other assays known in the art. In addition, BP sequence variants (assayed as single components or as BPXTEN fusion proteins) can be compared to the native BP using a competitive ELISA binding assay to determine whether they have the same binding specificity and affinity as the native BP, or some fraction thereof such that they are suitable for inclusion in BPXTEN.

The invention provides isolated BPXTEN in which the binding affinity for BP target receptors or ligands by the BPXTEN can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100% or more of the affinity of a native BP not bound to XTEN for the target receptor or ligand. In some cases, the binding affinity K_{d} between the subject BPXTEN and a native receptor or ligand of the BPXTEN is at least about 10⁻⁴ M, alternatively at least about 10⁻⁵ M, alternatively at least about 10⁻⁶ M, or at least about 10⁻⁷ M of the affinity between the BPXTEN and a native receptor or ligand.

In some embodiments, where a composition of this disclosure (such as a fusion protein) comprises a cytokine, a binding activity of the cytokine (when linked to an XTEN in the fusion protein) to a corresponding cytokine receptor can be characterized by a half maximal effective concentration (EC50) at least (about) 1.1 fold greater, at least (about) 1.2 fold greater, at least (about) 1.3 fold greater, at least (about) 1.4 fold greater, at least (about) 1.5 fold greater, at least (about) 1.6 fold greater, at least (about) 1.7 fold greater, at least (about) 1.8 fold greater, at least (about) 1.9 fold greater, or at least (about) 2.0 fold greater than an EC50 characterizing a corresponding binding activity of the cytokine (when not linked to the XTEN). In some embodiments, a binding activity of the cytokine (when linked to an XTEN in the fusion protein) to a corresponding cytokine receptor can be characterized by a half maximal effective concentration (EC50) of (about) 1.1 fold greater, (about) 1.2 fold greater, (about) 1.3 fold greater, (about) 1.4 fold greater, (about) 1.5 fold greater, (about) 1.6 fold greater, (about) 1.7 fold greater, (about) 1.8 fold greater, (about) 1.9 fold greater, or (about) 2.0 fold greater, or a range between any two of the foregoing, than an EC50 characterizing a corresponding binding activity of the cytokine (when not linked to the XTEN). In some embodiments, the EC50 value(s) can be determined in an in vitro binding assay. In some embodiments, the cytokine can be interleukin 12 (IL-12), and the corresponding cytokine receptor can be an interleukin 12 receptor (IL-12R). In some embodiments, the in vitro binding assay can utilize a genetically engineered reporter gene cell line configured to respond to binding of the cytokine to the corresponding cytokine receptor with a proportional expression of a reporter protein. The term “EC₅₀” generally refers to the concentration needed to achieve half of the maximum biological response of the active substance, and can be generally determined by ELISA or cell-based assays, including the methods of the Examples described herein. In some embodiments, the in vitro binding assay can be a reporter gene activity assay (such as one disclosed in Example 8). For example, an exemplary reporter gene activity assay can be based on genetically engineered cell(s), generated by stably introducing relevant gene(s) for the receptor(s)-of-interest and the signaling pathway(s)-of-interest, such that binding to the engineered receptor triggers a signaling cascade leading to the activation of the engineered gene pathway with a subsequent production of signature polypeptide(s) (such as an enzyme).

In other cases, the invention provides isolated BPXTEN in which the fusion protein is designed to bind with high affinity to a target receptor, thereby resulting in antagonistic activity for the native ligand. A non-limiting example of such a BPXTEN is IL-1raXTEN, which is configured to bind to an IL-1 receptor such that the bound composition substantially interferes with the binding of IL-1 α and/or IL-1β to IL-1 receptor. In certain cases, the interference by an antagonist BPXTEN (such as, but not limited to IL-1raXTEN) with the binding the native ligand to the target receptor can be at least about 1%, or about 10%, or about 20%, or about 30%, or about 40%, or about 50%, or about 60%, or about 70%, or about 80%, or about 90%, or about 95%, or about 99%, or about 100%. In other embodiments, the invention provides isolated BPXTEN fusion proteins (such as, but not limited to IL-1raXTEN) wherein the binding of the isolated fusion protein to a cellular receptor elicits less than 20%, or less than 10%, or less than 5% activation of the signaling pathways of the cell with bound BPXTEN antagonist in comparison to those evoked by the native ligand. In other cases, the antagonistic BPXTEN compositions bind to the target receptor with a dissociation constant of about 10 nM or less, about 5 nM or less, about 1 nM or less, about 500 μM or less, about 250 μM or less, about 100 μM or less, about 50 μM or less, or about 25 μM or less. Non-limiting examples of specific constructs of antagonistic BPXTEN can include IL-1ra-AM875, IL-1ra-AE864, or IL-1ra-AM1296.

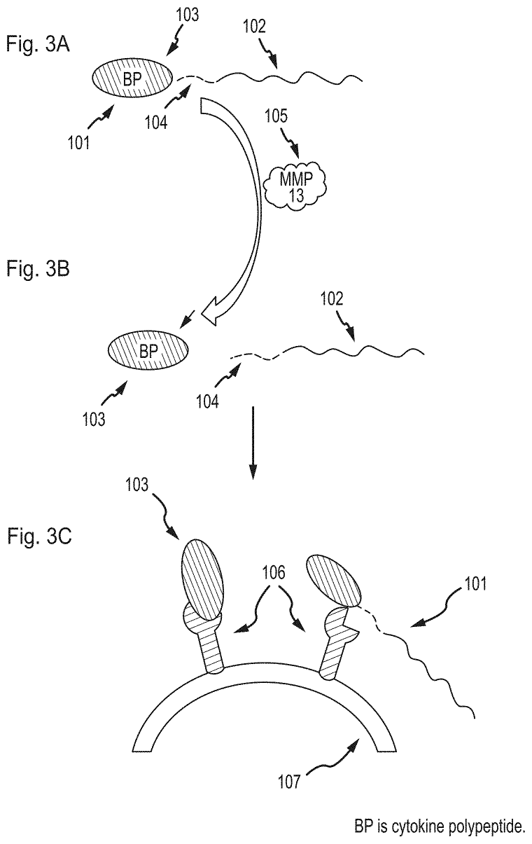

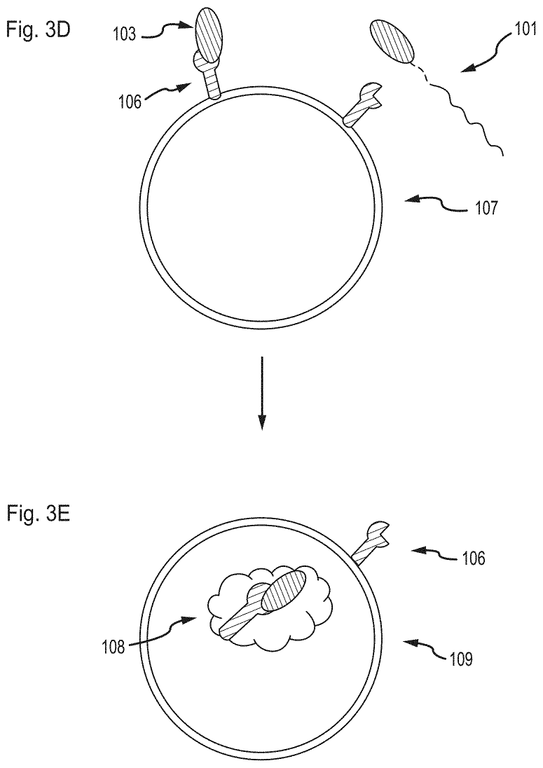

In some cases, the BPXTEN fusion proteins of the invention retain at least about 10%, or about 20%, 30%, 40%, 50%, 60%, 70%, 80%, 90%, 95%, 98%, or 99% percent of the biological activity of the corresponding BP not linked to the fusion protein with regard to an in vitro biologic activity or pharmacologic effect known or associated with the use of the native BP in the treatment and prevention of metabolic conditions and disorders. In some cases of the foregoing embodiment, the activity of the BP component may be manifest by the intact BPXTEN fusion protein, while in other cases the activity of the BP component would be primarily manifested upon cleavage and release of the BP from the fusion protein by action of a protease that acts on a cleavage sequence incorporated into the BPXTEN fusion protein. In the foregoing, as illustrated in FIG. 3A-FIG. 3E, the BPXTEN can be designed to reduce the binding affinity of the BP component for the receptor or ligand when linked to the XTEN but have increased affinity when released from XTEN through the cleavage of cleavage sequence(s) incorporated into the BPXTEN sequence, as described more fully above.

In other cases, the BPXTEN are designed to reduce the binding affinity of the BP component when linked to the XTEN to, for example, increase the terminal half-life of BPXTEN administered to a subject by reducing receptor-mediated clearance or to reduce toxicity or side effects due to the administered composition. Where the toxicological no-effect dose or blood concentration of a BP not linked to an XTEN is low (meaning that the native peptide has a high potential to result in side effects), the invention provides BPXTEN fusion proteins in which the fusion protein is configured to reduce the biologic potency or activity of the BP component.

In some cases, it has been found that a BPXTEN can be configured to have a substantially reduced binding affinity (expressed as Kd) and a corresponding reduced bioactivity, compared to the activity of a BPXTEN wherein the configuration does not result in reduced binding affinity of the corresponding BP component, and that such configuration is advantageous in terms of having a composition that displays both a long terminal half-life and retains a sufficient degree of bioactivity. Linking a single XTEN to the C-terminus of a BP (e.g., IL-10) can result in the retention of significant binding affinity to its target receptor, linking an XTEN to the N-terminus decreases its binding affinity and corresponding biological activity, compared to constructs where the XTEN is bound to the C-terminus. In another example, it has been found, as described in the Examples, that while linking of BP to the C-terminus of an XTEN molecule does not substantially interfere with the binding to the BP receptors, the addition of a second XTEN to the C-terminus of the same molecule (placing the second XTEN to the C-terminus of hGH) reduced the affinity of the molecule to the BP receptor and also resulted in an increase in terminal half-life of the XTEN-BP-XTEN configuration compared to XTEN-BP configuration. The ability to reduce binding affinity of the BP to its target receptor may be dependent on the requirement to have a free N- or C-terminus for the particular BP. Accordingly, the invention provides a method for increasing the terminal half-life of a BPXTEN by producing a single-chain fusion protein construct with a specific N- to C-terminus configuration of the components comprising at least a first biologically active protein and one or more XTEN, wherein the fusion protein in a first N- to C-terminus configuration of the biologically active protein and XTEN components has reduced receptor-mediated clearance (RMC) and a corresponding increase in terminal half-life compared to a BPXTEN in a second N- to C-terminus configuration. In one embodiment of the foregoing, the BPXTEN is configured, N- to C-terminus as BP-XTEN. In another embodiment of the foregoing, the BPXTEN is configured XTEN-BP. In another embodiment of the foregoing, the BPXTEN is configured XTEN-BP-XTEN. In the latter embodiment, the two XTEN molecules can be identical or they can be of a different sequence composition or length. Non-limiting examples of the foregoing embodiment with two XTEN linked to a single BP. Non-limiting examples of the foregoing embodiment with one BP linked to one XTEN include AM875-IL-1ra, AE864-IL-1ra, AM875-IL10, or AE864-IL10. The invention contemplates other such constructs in which a BP from Table 3 or Table A and XTEN from Tables 2a-2b are substituted for the respective components of the foregoing examples, and configured such that the construct has reduced receptor mediated clearance compared to an alternate configuration of the respective components.

In some cases, the method provides configured BPXTEN in which the reduced receptor mediated clearance can result in an increase in the terminal half-life of at least two-fold, or at least three-fold, or at least four-fold, or at least five-fold compared to the half-life of a BPXTEN in a second configuration where RMC is not reduced. The invention takes advantage of BP ligands wherein reduced binding affinity to a receptor, either as a result of a decreased on-rate or an increased off-rate, may be effected by the obstruction of either the N- or C-terminus, and using that terminus as the linkage to another polypeptide of the composition, whether another BP, an XTEN, or a spacer sequence. The choice of the particular configuration of the BPXTEN fusion protein can reduce the degree of binding affinity to the receptor such that a reduced rate of receptor-mediated clearance can be achieved. Generally, activation of the receptor is coupled to RMC such that binding of a polypeptide to its receptor without activation does not lead to RMC, while activation of the receptor leads to RMC. However, in some cases, particularly where the ligand has an increased off rate, the ligand may nevertheless be able to bind sufficiently to initiate cell signaling without triggering receptor mediated clearance, with the net result that the BPXTEN remains bioavailable. In such cases, the configured BPXTEN has an increased half-life compared to those configurations that lead to a higher degree of RMC.

In cases where a reduction in binding affinity is desired in order to reduce receptor-mediated clearance but retention of at least a portion of the biological activity is desired, it will be clear that sufficient binding affinity to obtain the desired receptor activation must nevertheless be maintained. Thus, in one embodiment, the invention provides a BPXTEN configured such that the binding affinity of the BPXTEN for a target receptor is in the range of about 0.01%-40%, or about 0.1%-30%, or about 1%-20% of the binding affinity compared to a corresponding BPXTEN in a configuration wherein the binding affinity is not reduced. The binding affinity of the configured BXTEN is thus preferably reduced by at least about 80%, or at least about 85%, or at least about 90%, or at least about 95%, or at least about 99%, or at least about 99.9%, or at least about 99.99% as compared to the binding affinity of a corresponding BPXTEN in a configuration wherein the binding affinity of the BP component to the target receptor is not reduced or compared to the BP not linked to the fusion protein, determined under comparable conditions. Expressed differently, the BP component of the configured BPXTEN may have a binding affinity that is as small as about 0.01%, or at least about 0.1%, or at least about 1%, or at least about 2%, or at least about 3%, or at least about 4%, or at least about 5%, or at least about 10%, or at least about 20% of that of the corresponding BP component of a BPXTEN in a configuration wherein the binding affinity of the BP component is not reduced. In the foregoing embodiments hereinabove described in this paragraph, the binding affinity of the configured BPXTEN for the target receptor would be “substantially reduced” compared to a corresponding native BP or a BPXTEN with a configuration in which the binding affinity of the corresponding BP component is not reduced. Accordingly, the present invention provides compositions and methods to produce compositions with reduced RMC by configuring the BPXTEN so as to be able to bind and activate a sufficient number of receptors to obtain a desired in vivo biological response yet avoid activation of more receptors than is required for obtaining such response. In one embodiment, the BPXTEN is configured such that the subject BP is at the N-terminus of the BPXTEN, wherein the RMC of the administered BPXTEN is reduced compared to a BPXTEN configured with the subject BP linked to the C-terminus of an XTEN and at least a portion of the biological activity of the native BP is retained. In another embodiment, the BPXTEN is configured such that the subject BP is at the C-terminus of the BPXTEN, wherein the RMC of the administered BPXTEN is reduced compared to a BPXTEN configured with the subject BP is at the N-terminus of the BPXTEN and at least a portion of the biological activity of the native BP is retained. In another embodiment, the BPXTEN is configured, N- to C-terminus, as XTEN-BP-XTEN, wherein the RMC of the administered BPXTEN is reduced compared to a BPXTEN configured with one XTEN and at least a portion of the biological activity of the native BP is retained. It will be apparent to one of skill in the art that other configurations to achieve this property are contemplated by the invention; e.g., addition of a second molecule of the BP or a spacer sequence. In the foregoing embodiments hereinabove described in this paragraph, the half-life of the BPXTEN can be increased at least about 50%, or at least about 75%, or at least about 100%, or at least about 150%, or at least about 200%, or at least about 300% compared to a BPXTEN configured wherein the binding affinity and RMC of the BP component is not reduced. In the foregoing embodiments hereinabove described in this paragraph, the increased half-life can permit higher dosages and reduced frequency of dosing compared to BP not linked to XTEN or compared to BPXTEN configurations wherein the BP component retains a binding affinity to the receptor comparable to the native BP.

Specific in vivo and ex vivo biological assays may also be used to assess the biological activity of each configured BPXTEN and/or BP component to be incorporated into BPXTEN. For example, the increase of insulin secretion and/or transcription from the pancreatic beta cells can be measured by methods known in the art. Glucose uptake by tissues can also be assessed by methods such as the glucose clamp assay and the like. Other in vivo and ex vivo parameters suitable to assess the activity of administered BPXTEN fusion proteins in treatment of metabolic diseases and disorders include fasting glucose level, peak postprandial glucose level, glucose homeostasis, response to oral glucose tolerance test, response to insulin challenge, HAic, caloric intake, satiety, rate of gastric emptying, pancreatic secretion, insulin secretion, peripheral tissue insulin sensitivity, beta cell mass, beta cell destruction, blood lipid levels or profiles, body mass index, or body weight. Based on the results of these assays or other assays known in the art, the BPXTEN configuration or composition can be confirmed or, if needed, adjusted and re-assayed to confirm the target binding affinity or biologic activity.

Specific assays and methods for measuring the physical and structural properties of expressed proteins are known in the art, including methods for determining properties such as protein aggregation, solubility, secondary and tertiary structure, melting properties, contamination and water content, etc. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Arnau et al, Prot Expr and Purif (2006) 48, 1-13. Application of these methods to the invention would be within the grasp of a person skilled in the art.

### Uses of the Compositions of the Present Invention

In another aspect, the invention provides a method of for achieving a beneficial effect in a disease, disorder or condition mediated by BP. The present invention addresses disadvantages and/or limitations of BP that have a relatively short terminal half-life and/or a narrow therapeutic window between the minimum effective dose and the maximum tolerated dose.

In one embodiment, the invention provides a method for achieving a beneficial effect in a subject comprising the step of administering to the subject a therapeutically- or prophylactically-effective amount of a BPXTEN. The effective amount can produce a beneficial effect in helping to treat a disease or disorder. In some cases, the method for achieving a beneficial effect can include administering a therapeutically effective amount of a BPXTEN fusion protein composition to treat a subject with.

In one embodiment, the method comprises administering a therapeutically-effective amount of a pharmaceutical composition comprising a BPXTEN fusion protein composition comprising a BP linked to an XTEN sequence(s) and at least one pharmaceutically acceptable carrier to a subject in need thereof that results in greater improvement in at least one parameter, physiologic condition, or clinical outcome mediated by the BP component(s) compared to the effect mediated by administration of a pharmaceutical composition comprising a BP not linked to XTEN and administered at a comparable dose. In one embodiment, the pharmaceutical composition is administered at a therapeutically effective dose. In another embodiment, the pharmaceutical composition is administered using multiple consecutive doses using a therapeutically effective dose regimen (as defined herein) for the length of the dosing period.

As a result of the enhanced PK parameters of BPXTEN, as described herein, the BP may be administered using longer intervals between doses compared to the corresponding BP not linked to XTEN to prevent, treat, alleviate, reverse or ameliorate symptoms or clinical abnormalities of the metabolic disease, disorder or condition or prolong the survival of the subject being treated.

The methods of the invention may include administration of consecutive doses of a therapeutically effective amount of the BPXTEN for a period of time sufficient to achieve and/or maintain the desired parameter or clinical effect, and such consecutive doses of a therapeutically effective amount establishes the therapeutically effective dose regimen for the BPXTEN; e.g., the schedule for consecutively administered doses of the fusion protein composition, wherein the doses are given in therapeutically effective amounts to result in a sustained beneficial effect on any clinical sign or symptom, aspect, measured parameter or characteristic of a metabolic disease state or condition, including, but not limited to, those described herein.

A therapeutically effective amount of the BPXTEN may vary according to factors such as the disease state, age, sex, and weight of the individual, and the ability of the antibody or antibody portion to elicit a desired response in the individual. A therapeutically effective amount is also one in which any toxic or detrimental effects of the BPXTEN are outweighed by the therapeutically beneficial effects. A prophylactically effective amount refers to an amount of BPXTEN required for the period of time necessary to achieve the desired prophylactic result.

For the inventive methods, longer acting BPXTEN compositions are preferred, so as to improve patient convenience, to increase the interval between doses and to reduce the amount of drug required to achieve a sustained effect. In one embodiment, a method of treatment comprises administration of a therapeutically effective dose of a BPXTEN to a subject in need thereof that results in a gain in time spent within a therapeutic window established for the fusion protein of the composition compared to the corresponding BP component(s) not linked to the fusion protein and administered at a comparable dose to a subject. In some cases, the gain in time spent within the therapeutic window is at least about three-fold, or at least about four-fold, or at least about five-fold, or at least about six-fold, or at least about eight-fold, or at least about 10-fold, or at least about 20-fold, or at least about 40-fold compared to the corresponding BP component not linked to the fusion protein and administered at a comparable dose to a subject. The methods further provide that administration of multiple consecutive doses of a BPXTEN administered using a therapeutically effective dose regimen to a subject in need thereof can result in a gain in time between consecutive Cₘₐₓ peaks and/or Cₘᵢₙ troughs for blood levels of the fusion protein compared to the corresponding BP(s) not linked to the fusion protein and administered using a dose regimen established for that BP. In the foregoing embodiment, the gain in time spent between consecutive Cₘₐₓ peaks and/or Cₘᵢₙ troughs can be at least about three-fold, or at least about four-fold, or at least about five-fold, or at least about six-fold, or at least about eight-fold, or at least about 10-fold, or at least about 20-fold, or at least about 40-fold compared to the corresponding BP component(s) not linked to the fusion protein and administered using a dose regimen established for that BP. In the embodiments hereinabove described in this paragraph the administration of the fusion protein can result in an improvement in at least one of the parameters (disclosed herein as being useful for assessing the subject diseases, conditions or disorders) using a lower unit dose in moles of fusion protein compared to the corresponding BP component(s) not linked to the fusion protein and administered at a comparable unit dose or dose regimen to a subject.

In one embodiment, the BPXTEN can have activity that results in an improvement in one of the clinical, biochemical or physiologic parameters that is greater than the activity of the BP component not linked to XTEN, determined using the same assay or based on a measured clinical parameter. In another embodiment, the BPXTEN can have activity in two or more clinical or metabolic-related parameters (e.g., glucose homeostasis and weight control in a diabetic subject, or reduced prothrombin and bleeding times in a hemophiliac subject, or increased muscle mass and bone density in a growth-hormone deficient subject), each mediated by one of the different BP that collectively result in an enhanced effect compared the BP component not linked to XTEN, determined using the same assays or based on measured clinical parameters. In another embodiment, administration of the BPXTEN can result in activity in one or more of the clinical or biochemical or physiologic parameters that is of longer duration than the activity of one of the single BP components not linked to XTEN, determined using that same assay or based on a measured clinical parameter.

In some embodiments, the present disclosure provides a method of treating or preventing a disease or condition in a subject, the method comprising administering to a subject a therapeutically effective amount of a fusion protein or a composition comprising the fusion protein, all of which are disclosed herein. In some embodiments, the disease or condition can be a cancer, or a cancer-related disease or condition, or an inflammatory or autoimmune disease. In some embodiments, the disease or condition can be a cancer, or a cancer-related disease or condition. In some embodiments, the disease or condition can be a cancer or a cancer-related disease or condition. Where desired, the subject fusion and composition can be used in conjunction with a therapeutically effective amount of at least one immune checkpoint inhibitor.

The invention further contemplates that BPXTEN used in accordance with the methods provided herein may be administered in conjunction with other treatment methods and pharmaceutical compositions useful for treating cancer, rheumatoid arthritis, multiple sclerosis, myasthenia gravis, systemic lupus erythematosus, Alzheimer's disease, Schizophrenia, viral infections (e.g., chronic hepatitis C, AIDS), allergic asthma, retinal neurodegenerative processes, metabolic disorder, insulin resistance, and diabetic cardiomyopathy. inflammatory conditions and autoimmune conditions.

In some cases, the administration of a BPXTEN may permit use of lower dosages of the co-administered pharmaceutical composition to achieve a comparable clinical effect or measured parameter for the disease, disorder or condition in the subject.

The foregoing notwithstanding, in certain embodiments, the BPXTEN used in accordance with the methods of the present invention may prevent or delay the need for additional treatment methods or use of drugs or other pharmaceutical compositions in subjects with glucose-related diseases, metabolic diseases or disorders, coagulation disorders, or growth-hormone deficiency or growth disorders. In other embodiments, the BPXTEN may reduce the amount, frequency or duration of additional treatment methods or drugs or other pharmaceutical compositions required to treat the underlying disease, disorder or condition.

In another aspect, the invention provides a method of designing the BPXTEN compositions with desired pharmacologic or pharmaceutical properties. The BPXTEN fusion proteins are designed and prepared with various objectives in mind (compared to the BP components not linked to the fusion protein), including improving the therapeutic efficacy for the treatment of metabolic diseases or disorders, enhancing the pharmacokinetic characteristics of the fusion proteins compared to the BP, lowering the dose or frequency of dosing required to achieve a pharmacologic effect, enhancing the pharmaceutical properties, and to enhance the ability of the BP components to remain within the therapeutic window for an extended period of time.

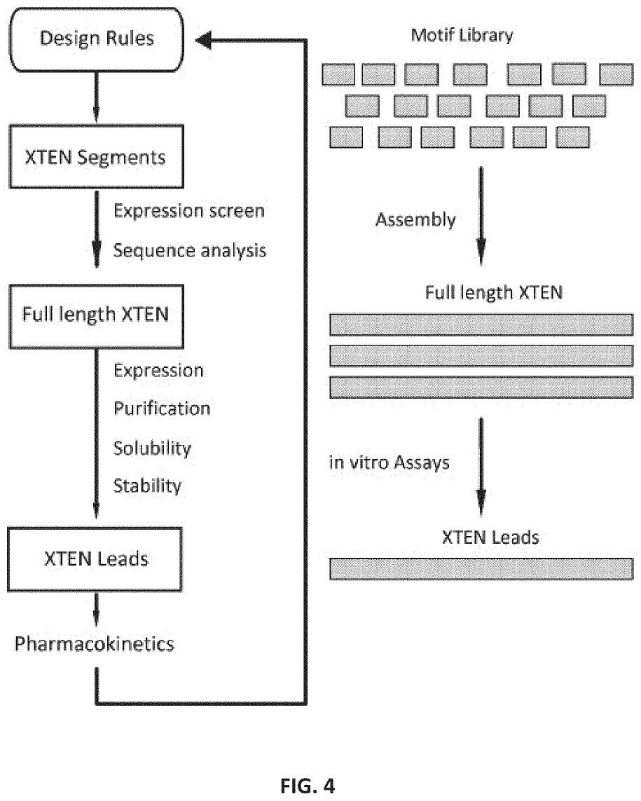

In general, the steps in the design and production of the fusion proteins and the inventive compositions may, as illustrated in FIGS. 4-6, include: (1) the selection of BPs (e.g., native proteins, peptide hormones, peptide analogs or derivatives with activity, peptide fragments, etc.) to treat the particular disease, disorder or condition; (2) selecting the XTEN that will confer the desired PK and physicochemical characteristics on the resulting BPXTEN (e.g., the administration of the composition to a subject results in the fusion protein being maintained within the therapeutic window for a greater period compared to BP not linked to XTEN); (3) establishing a desired N- to C-terminus configuration of the BPXTEN to achieve the desired efficacy or PK parameters; (4) establishing the design of the expression vector encoding the configured BPXTEN; (5) transforming a suitable host with the expression vector; and (6) expression and recovery of the resultant fusion protein. For those BPXTEN for which an increase in half-life (greater than 16 h) or an increased period of time spent within a therapeutic window is desired, the XTEN chosen for incorporation will generally have at least about 500, or about 576, or about 864, or about 875, or about 913, or about 924 amino acid residues where a single XTEN is to be incorporated into the BPXTEN. In another embodiment, the BPXTEN can comprise a first XTEN of the foregoing lengths, and a second XTEN of about 144, or about 288, or about 576, or about 864, or about 875, or about 913, or about 924 amino acid residues.

In other cases, where in increase in half-life is not required, but an increase in a pharmaceutical property (e.g., solubility) is desired, a BPXTEN can be designed to include XTEN of shorter lengths. In some embodiments of the foregoing, the BPXTEN can comprise a BP linked to an XTEN having at least about 24, or about 36, or about 48, or about 60, or about 72, or about 84, or about 96 amino acid residues, in which the solubility of the fusion protein under physiologic conditions is at least three-fold greater than the corresponding BP not linked to XTEN, or alternatively, at least four-fold, or five-fold, or six-fold, or seven-fold, or eight-fold, or nine-fold, or at least 10-fold, or at least 20-fold, or at least 30-fold, or at least 50-fold, or at least 60-fold or greater than glucagon not linked to XTEN. In still other cases, where a half-life of 2-6 hours for a glucagon-containing BPXTEN fusion protein is desired (e.g., in the treatment of nocturnal hypoglycemia), a fusion protein can be designed with XTEN of intermediate lengths such as about 100 amino acids, or about 144 amino acids, or about 156 amino acids, or about 168 amino acids, or about 180 amino acids, or about 196 amino acids in the XTEN component of the glucagon-containing BPXTEN.

In another aspect, the invention provides methods of making BPXTEN compositions to improve ease of manufacture, result in increased stability, increased water solubility, and/or ease of formulation, as compared to the native BPs. In one embodiment, the invention includes a method of increasing the water solubility of a BP comprising the step of linking the BP to one or more XTEN such that a higher concentration in soluble form of the resulting BPXTEN can be achieved, under physiologic conditions, compared to the BP in an un-fused state. Factors that contribute to the property of XTEN to confer increased water solubility of BPs when incorporated into a fusion protein include the high solubility of the XTEN fusion partner and the low degree of self-aggregation between molecules of XTEN in solution. In some embodiments, the method results in a BPXTEN fusion protein wherein the water solubility is at least about 50%, or at least about 60% greater, or at least about 70% greater, or at least about 80% greater, or at least about 90% greater, or at least about 100% greater, or at least about 150% greater, or at least about 200% greater, or at least about 400% greater, or at least about 600% greater, or at least about 800% greater, or at least about 1000% greater, or at least about 2000% greater, or at least about 4000% greater, or at least about 6000% greater under physiologic conditions, compared to the un-fused BP.

In another embodiment, the invention includes a method of enhancing the shelf-life of a BP comprising the step of linking the BP with one or more XTEN selected such that the shelf-life of the resulting BPXTEN is extended compared to the BP in an un-fused state. As used herein, shelf-life refers to the period of time over which the functional activity of a BP or BPXTEN that is in solution or in some other storage formulation remains stable without undue loss of activity. As used herein, “functional activity” refers to a pharmacologic effect or biological activity, such as the ability to bind a receptor or ligand, or an enzymatic activity, or to display one or more known functional activities associated with a BP, as known in the art. A BP that degrades or aggregates generally has reduced functional activity or reduced bioavailability compared to one that remains in solution. Factors that contribute to the ability of the method to extend the shelflife of BPs when incorporated into a fusion protein include the increased water solubility, reduced self-aggregation in solution, and increased heat stability of the XTEN fusion partner. In particular, the low tendency of XTEN to aggregate facilitates methods of formulating pharmaceutical preparations containing higher drug concentrations of BPs, and the heat-stability of XTEN contributes to the property of BPXTEN fusion proteins to remain soluble and functionally active for extended periods. In one embodiment, the method results in BPXTEN fusion proteins with “prolonged” or “extended” shelf-life that exhibit greater activity relative to a standard that has been subjected to the same storage and handling conditions. The standard may be the un-fused full-length BP. In one embodiment, the method includes the step of formulating the isolated BPXTEN with one or more pharmaceutically acceptable excipients that enhance the ability of the XTEN to retain its unstructured conformation and for the BPXTEN to remain soluble in the formulation for a time that is greater than that of the corresponding un-fused BP. In one embodiment, the method encompasses linking a BP to an XTEN to create a BPXTEN fusion protein results in a solution that retains greater than about 100% of the functional activity, or greater than about 1050%, 110%, 120%, 130%, 150% or 200% of the functional activity of a standard when compared at a given time point and when subjected to the same storage and handling conditions as the standard, thereby enhancing its shelf-life.

Shelf-life may also be assessed in terms of functional activity remaining after storage, normalized to functional activity when storage began. BPXTEN fusion proteins of the invention with prolonged or extended shelf-life as exhibited by prolonged or extended functional activity may retain about 50% more functional activity, or about 60%, 70%, 80%, or 90% more of the functional activity of the equivalent BP not linked to XTEN when subjected to the same conditions for the same period of time. For example, a BPXTEN fusion protein of the invention comprising exendin-4 or glucagon fused to a XTEN sequence may retain about 80% or more of its original activity in solution for periods of up to 5 weeks or more under various temperature conditions. In some embodiments, the BPXTEN retains at least about 50%, or about 60%, or at least about 70%, or at least about 80%, and most preferably at least about 90% or more of its original activity in solution when heated at 80° C. for 10 min. In other embodiments, the BPXTEN retains at least about 50%, preferably at least about 60%, or at least about 70%, or at least about 80%, or alternatively at least about 90% or more of its original activity in solution when heated or maintained at 37° C. for about 7 days. In another embodiment, BPXTEN fusion protein retains at least about 80% or more of its functional activity after exposure to a temperature of about 30° C. to about 70° C. over a period of time of about one hour to about 18 hours. In the foregoing embodiments hereinabove described in this paragraph, the retained activity of the BPXTEN would be at least about two-fold, or at least about three-fold, or at least about four-fold, or at least about five-fold, or at least about six-fold greater at a given time point than that of the corresponding BP not linked to the fusion protein.

### The DNA Sequences of the Invention

The present invention provides isolated polynucleic acids encoding BPXTEN chimeric polypeptides and sequences complementary to polynucleic acid molecules encoding BPXTEN chimeric polypeptides, including homologous variants. In another aspect, the invention encompasses methods to produce polynucleic acids encoding BPXTEN chimeric polypeptides and sequences complementary to polynucleic acid molecules encoding BPXTEN chimeric polypeptides, including homologous variants. In general, and as illustrated in FIGS. 4-6, the methods of producing a polynucleotide sequence coding for a BPXTEN fusion protein and expressing the resulting gene product include assembling nucleotides encoding BP and XTEN, linking the components in frame, incorporating the encoding gene into an appropriate expression vector, transforming an appropriate host cell with the expression vector, and causing the fusion protein to be expressed in the transformed host cell, thereby producing the biologically-active BPXTEN polypeptide. Standard recombinant techniques in molecular biology can be used to make the polynucleotides and expression vectors of the present invention.

In accordance with the invention, nucleic acid sequences that encode BPXTEN may be used to generate recombinant DNA molecules that direct the expression of BPXTEN fusion proteins in appropriate host cells. Several cloning strategies are envisioned to be suitable for performing the present invention, many of which can be used to generate a construct that comprises a gene coding for a fusion protein of the BPXTEN composition of the present invention, or its complement. In one embodiment, the cloning strategy would be used to create a gene that encodes a monomeric BPXTEN that comprises at least a first BP and at least a first XTEN polypeptide, or its complement. In another embodiment, the cloning strategy would be used to create a gene that encodes a monomeric BPXTEN that comprises a first and a second molecule of the one BP and at least a first XTEN (or its complement) that would be used to transform a host cell for expression of the fusion protein used to formulate a BPXTEN composition. In the foregoing embodiments hereinabove described in this paragraph, the gene can further comprise nucleotides encoding spacer sequences that may also encode cleavage sequence(s).

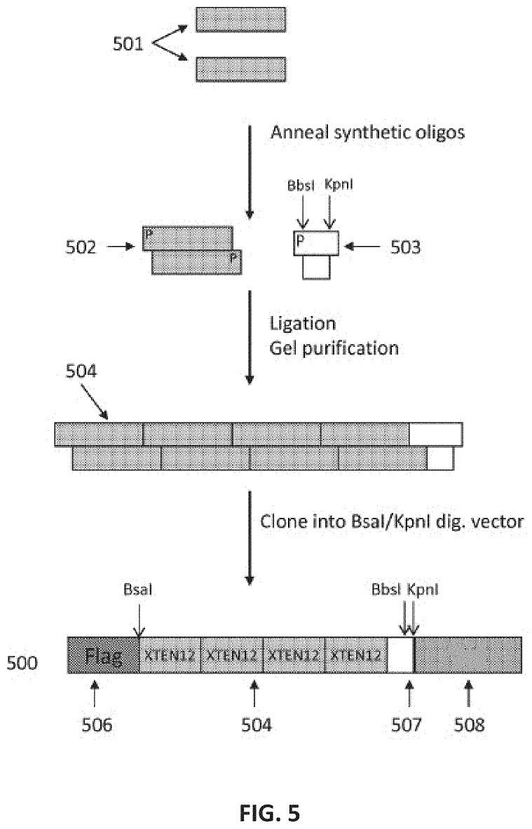

In designing a desired XTEN sequences, it was discovered that the non-repetitive nature of the XTEN of the inventive compositions can be achieved despite use of a “building block” molecular approach in the creation of the XTEN-encoding sequences. This was achieved by the use of a library of polynucleotides encoding sequence motifs that are then multimerized to create the genes encoding the XTEN sequences (see FIGS. 4 and 5). Thus, while the expressed XTEN may consist of multiple units of as few as four different sequence motifs, because the motifs themselves consist of non-repetitive amino acid sequences, the overall XTEN sequence is rendered non-repetitive. Accordingly, in one embodiment, the XTEN-encoding polynucleotides comprise multiple polynucleotides that encode non-repetitive sequences, or motifs, operably linked in frame and in which the resulting expressed XTEN amino acid sequences are non-repetitive.

In one approach, a construct is first prepared containing the DNA sequence corresponding to BPXTEN fusion protein. DNA encoding the BP of the compositions may be obtained from a cDNA library prepared using standard methods from tissue or isolated cells believed to possess BP mRNA and to express it at a detectable level. If necessary, the coding sequence can be obtained using conventional primer extension procedures as described in Sambrook, et al., supra, to detect precursors and processing intermediates of mRNA that may not have been reverse-transcribed into cDNA. Accordingly, DNA can be conveniently obtained from a cDNA library prepared from such sources. The BP encoding gene(s) may also be obtained from a genomic library or created by standard synthetic procedures known in the art (e.g., automated nucleic acid synthesis) using DNA sequences obtained from publicly available databases, patents, or literature references. Such procedures are well known in the art and well described in the scientific and patent literature. For example, sequences can be obtained from Chemical Abstracts Services (CAS) Registry Numbers (published by the American Chemical Society) and/or GenBank Accession Numbers (e.g., Locus ID, NP_XXXXX, and XP_XXXXX) Model Protein identifiers available through the National Center for Biotechnology Information (NCBI) webpage, available on the world wide web at ncbi.nlm.nih.gov that correspond to entries in the CAS Registry or GenBank database that contain an amino acid sequence of the BAP or of a fragment or variant of the BAP. For such sequence identifiers provided herein, the summary pages associated with each of these CAS and GenBank and GenSeq Accession Numbers as well as the cited journal publications (e.g., PubMed ID number (PMID)) are each incorporated by reference in their entireties, particularly with respect to the amino acid sequences described therein. In one embodiment, the BP encoding gene encodes a protein from any one of Table 3 or Table A, or a fragment or variant thereof.

A gene or polynucleotide encoding the BP portion of the subject BPXTEN protein, in the case of an expressed fusion protein that will comprise a single BP can then be cloned into a construct, which can be a plasmid or other vector under control of appropriate transcription and translation sequences for high level protein expression in a biological system. In a later step, a second gene or polynucleotide coding for the XTEN is genetically fused to the nucleotides encoding the N- and/or C-terminus of the BP gene by cloning it into the construct adjacent and in frame with the gene(s) coding for the BP. This second step can occur through a ligation or multimerization step. In the foregoing embodiments hereinabove described in this paragraph, it is to be understood that the gene constructs that are created can alternatively be the complement of the respective genes that encode the respective fusion proteins.

The gene encoding for the XTEN can be made in one or more steps, either fully synthetically or by synthesis combined with enzymatic processes, such as restriction enzyme-mediated cloning, PCR and overlap extension. XTEN polypeptides can be constructed such that the XTEN-encoding gene has low repetitiveness while the encoded amino acid sequence has a degree of repetitiveness. Genes encoding XTEN with non-repetitive sequences can be assembled from oligonucleotides using standard techniques of gene synthesis. The gene design can be performed using algorithms that optimize codon usage and amino acid composition. In one method of the invention, a library of relatively short XTEN-encoding polynucleotide constructs is created and then assembled, as illustrated in FIGS. 4 and 5. This can be a pure codon library such that each library member has the same amino acid sequence but many different coding sequences are possible. Such libraries can be assembled from partially randomized oligonucleotides and used to generate large libraries of XTEN segments comprising the sequence motifs. The randomization scheme can be optimized to control amino acid choices for each position as well as codon usage.

### Polynucleotide Libraries

In another aspect, the invention provides libraries of polynucleotides that encode XTEN sequences that can be used to assemble genes that encode XTEN of a desired length and sequence.

In certain embodiments, the XTEN-encoding library constructs comprise polynucleotides that encode polypeptide segments of a fixed length. As an initial step, a library of oligonucleotides that encode motifs of 9-14 amino acid residues can be assembled. In a preferred embodiment, libraries of oligonucleotides that encode motifs of 12 amino acids are assembled.

The XTEN-encoding sequence segments can be dimerized or multimerized into longer encoding sequences. Dimerization or multimerization can be performed by ligation, overlap extension, PCR assembly or similar cloning techniques known in the art. This process of can be repeated multiple times until the resulting XTEN-encoding sequences have reached the organization of sequence and desired length, providing the XTEN-encoding genes. As will be appreciated, a library of polynucleotides that encodes 12 amino acids can be dimerized into a library of polynucleotides that encode 36 amino acids. In turn, the library of polynucleotides that encode 36 amino acids can be serially dimerized into a library containing successively longer lengths of polynucleotides that encode XTEN sequences. In some embodiments, libraries can be assembled of polynucleotides that encode amino acids that are limited to specific sequence XTEN families; e.g., AD, AE, AF, AG, AM, or AQ sequences of Table 1. In other embodiments, libraries can comprise sequences that encode two or more of the motif family sequences from Table 1. The libraries can be used, in turn, for serial dimerization or ligation to achieve polynucleotide sequence libraries that encode XTEN sequences, for example, of 72, 144, 288, 576, 864, 912, 923, 1296 amino acids, or up to a total length of about 3000 amino acids, as well as intermediate lengths. In some cases, the polynucleotide library sequences may also include additional bases used as “sequencing islands,” described more fully below.

FIG. 5 is a schematic flowchart of representative, non-limiting steps in the assembly of a XTEN polynucleotide construct and a BPXTEN polynucleotide construct in the embodiments of the invention. Individual oligonucleotides 501 can be annealed into sequence motifs 502 such as a 12 amino acid motif (“12-mer”), which is subsequently ligated with an oligo containing BbsI, and KpnI restriction sites 503. Additional sequence motifs from a library are annealed to the 12-mer until the desired length of the XTEN gene 504 is achieved. The XTEN gene is cloned into a stuffer vector. The vector can optionally encode a Flag sequence 506 followed by a stuffer sequence that is flanked by BsaI, BbsI, and KpnI sites 507 and, in this case, a single BP gene (encoding exendin-4 in this example) 508, resulting in the gene encoding a BPXTEN comprising a single BP 500. A non-exhaustive list of the XTEN names and SEQ ID NOS. for polynucleotides encoding XTEN and precursor sequences is provided in Table 8.

TABLE 8
DNA sequences of XTEN and precursor sequences

| XTEN | SEQ ID | |
|---|---|---|
| Name | NO: | DNA Sequence |
| AE144 | 247 | GGTAGCGAACCGGCAACTTCCGGCTCTGAAACCCCAGGTACTTCTGAAAGCGCTACTC |
| | | CTGAGTCTGGCCCAGGTAGCGAACCTGCTACCTCTGGCTCTGAAACCCCAGGTAGCCC |
| | | GGCAGGCTCTCCGACTTCCACCGAGGAAGGTACCTCTACTGAACCTTCTGAGGGTAGC |
| | | GCTCCAGGTAGCGAACCGGCAACCTCTGGCTCTGAAACCCCAGGTAGCGAACCTGCTA |
| | | CCTCCGGCTCTGAAACTCCAGGTAGCGAACCGGCTACTTCCGGTTCTGAAACTCCAGG |
| | | TACCTCTACCGAACCTTCCGAAGGCAGCGCACCAGGTACTTCTGAAAGCGCAACCCCT |
| | | GAATCCGGTCCAGGTAGCGAACCGGCTACTTCTGGCTCTGAGACTCCAGGTACTTCTA |
| | | CCGAACCGTCCGAAGGTAGCGCACCA |
| AF144 | 248 | GGTACTTCTACTCCGGAAAGCGGTTCCGCATCTCCAGGTACTTCTCCTAGCGGTGAAT |
| | | CTTCTACTGCTCCAGGTACCTCTCCTAGCGGCGAATCTTCTACTGCTCCAGGTTCTAC |
| | | CAGCTCTACCGCTGAATCTCCTGGCCCAGGTTCTACCAGCGAATCCCCGTCTGGCACC |
| | | GCACCAGGTTCTACTAGCTCTACCGCAGAATCTCCGGGTCCAGGTACTTCCCCTAGCG |
| | | GTGAATCTTCTACTGCTCCAGGTACCTCTACTCCGGAAAGCGGCTCCGCATCTCCAGG |
| | | TTCTACTAGCTCTACTGCTGAATCTCCTGGTCCAGGTACCTCCCCTAGCGGCGAATCT |
| | | TCTACTGCTCCAGGTACCTCTCCTAGCGGCGAATCTTCTACCGCTCCAGGTACCTCCC |
| | | CTAGCGGTGAATCTTCTACCGCACCA |
| AE288 | 249 | GGTACCTCTGAAAGCGCAACTCCTGAGTCTGGCCCAGGTAGCGAACCTGCTACCTCCG |
| | | GCTCTGAGACTCCAGGTACCTCTGAAAGCGCAACCCCGGAATCTGGTCCAGGTAGCGA |
| | | ACCTGCAACCTCTGGCTCTGAAACCCCAGGTACCTCTGAAAGCGCTACTCCTGAATCT |
| | | GGCCCAGGTACTTCTACTGAACCGTCCGAGGGCAGCGCACCAGGTAGCCCTGCTGGCT |
| | | CTCCAACCTCCACCGAAGAAGGTACCTCTGAAAGCGCAACCCCTGAATCCGGCCCAGG |
| | | TAGCGAACCGGCAACCTCCGGTTCTGAAACCCCAGGTACTTCTGAAAGCGCTACTCCT |
| | | GAGTCCGGCCCAGGTAGCCCGGCTGGCTCTCCGACTTCCACCGAGGAAGGTAGCCCGG |
| | | CTGGCTCTCCAACTTCTACTGAAGAAGGTACTTCTACCGAACCTTCCGAGGGCAGCGC |
| | | ACCAGGTACTTCTGAAAGCGCTACCCCTGAGTCCGGCCCAGGTACTTCTGAAAGCGCT |
| | | ACTCCTGAATCCGGTCCAGGTACTTCTGAAAGCGCTACCCCGGAATCTGGCCCAGGTA |
| | | GCGAACCGGCTACTTCTGGTTCTGAAACCCCAGGTAGCGAACCGGCTACCTCCGGTTC |
| | | TGAAACTCCAGGTAGCCCAGCAGGCTCTCCGACTTCCACTGAGGAAGGTACTTCTACT |
| | | GAACCTTCCGAAGGCAGCGCACCAGGTACCTCTACTGAACCTTCTGAGGGCAGCGCTC |
| | | CAGGTAGCGAACCTGCAACCTCTGGCTCTGAAACCCCAGGTACCTCTGAAAGCGCTAC |
| | | TCCTGAATCTGGCCCAGGTACTTCTACTGAACCGTCCGAGGGCAGCGCACCA |
| AE576 | 250 | GGTAGCCCGGCTGGCTCTCCTACCTCTACTGAGGAAGGTACTTCTGAAAGCGCTACTC |
| | | CTGAGTCTGGTCCAGGTACCTCTACTGAACCGTCCGAAGGTAGCGCTCCAGGTAGCCC |
| | | AGCAGGCTCTCCGACTTCCACTGAGGAAGGTACTTCTACTGAACCTTCCGAAGGCAGC |
| | | GCACCAGGTACCTCTACTGAACCTTCTGAGGGCAGCGCTCCAGGTACTTCTGAAAGCG |
| | | CTACCCCGGAATCTGGCCCAGGTAGCGAACCGGCTACTTCTGGTTCTGAAACCCCAGG |
| | | TAGCGAACCGGCTACCTCCGGTTCTGAAACTCCAGGTAGCCCGGCAGGCTCTCCGACC |
| | | TCTACTGAGGAAGGTACTTCTGAAAGCGCAACCCCGGAGTCCGGCCCAGGTACCTCTA |
| | | CCGAACCGTCTGAGGGCAGCGCACCAGGTACTTCTACCGAACCGTCCGAGGGTAGCGC |
| | | ACCAGGTAGCCCAGCAGGTTCTCCTACCTCCACCGAGGAAGGTACTTCTACCGAACCG |
| | | TCCGAGGGTAGCGCACCAGGTACCTCTACTGAACCTTCTGAGGGCAGCGCTCCAGGTA |
| | | CTTCTGAAAGCGCTACCCCGGAGTCCGGTCCAGGTACTTCTACTGAACCGTCCGAAGG |
| | | TAGCGCACCAGGTACTTCTGAAAGCGCAACCCCTGAATCCGGTCCAGGTAGCGAACCG |
| | | GCTACTTCTGGCTCTGAGACTCCAGGTACTTCTACCGAACCGTCCGAAGGTAGCGCAC |
| | | CAGGTACTTCTACTGAACCGTCTGAAGGTAGCGCACCAGGTACTTCTGAAAGCGCAAC |
| | | CCCGGAATCCGGCCCAGGTACCTCTGAAAGCGCAACCCCGGAGTCCGGCCCAGGTAGC |
| | | CCTGCTGGCTCTCCAACCTCCACCGAAGAAGGTACCTCTGAAAGCGCAACCCCTGAAT |
| | | CCGGCCCAGGTAGCGAACCGGCAACCTCCGGTTCTGAAACCCCAGGTACCTCTGAAAG |
| | | CGCTACTCCGGAGTCTGGCCCAGGTACCTCTACTGAACCGTCTGAGGGTAGCGCTCCA |
| | | GGTACTTCTACTGAACCGTCCGAAGGTAGCGCACCAGGTACTTCTACCGAACCGTCCG |
| | | AAGGCAGCGCTCCAGGTACCTCTACTGAACCTTCCGAGGGCAGCGCTCCAGGTACCTC |
| | | TACCGAACCTTCTGAAGGTAGCGCACCAGGTACTTCTACCGAACCGTCCGAGGGTAGC |
| | | GCACCAGGTAGCCCAGCAGGTTCTCCTACCTCCACCGAGGAAGGTACTTCTACCGAAC |
| | | CGTCCGAGGGTAGCGCACCAGGTACCTCTGAAAGCGCAACTCCTGAGTCTGGCCCAGG |
| | | TAGCGAACCTGCTACCTCCGGCTCTGAGACTCCAGGTACCTCTGAAAGCGCAACCCCG |
| | | GAATCTGGTCCAGGTAGCGAACCTGCAACCTCTGGCTCTGAAACCCCAGGTACCTCTG |
| | | AAAGCGCTACTCCTGAATCTGGCCCAGGTACTTCTACTGAACCGTCCGAGGGCAGCGC |
| | | ACCAGGTACTTCTGAAAGCGCTACTCCTGAGTCCGGCCCAGGTAGCCCGGCTGGCTCT |
| | | CCGACTTCCACCGAGGAAGGTAGCCCGGCTGGCTCTCCAACTTCTACTGAAGAAGGTA |
| | | GCCCGGCAGGCTCTCCGACCTCTACTGAGGAAGGTACTTCTGAAAGCGCAACCCCGGA |
| | | GTCCGGCCCAGGTACCTCTACCGAACCGTCTGAGGGCAGCGCACCA |
| AF576 | 251 | GGTTCTACTAGCTCTACCGCTGAATCTCCTGGCCCAGGTTCCACTAGCTCTACCGCAG |
| | | AATCTCCGGGCCCAGGTTCTACTAGCGAATCCCCTTCTGGTACCGCTCCAGGTTCTAC |
| | | TAGCTCTACCGCTGAATCTCCGGGTCCAGGTTCTACCAGCTCTACTGCAGAATCTCCT |
| | | GGCCCAGGTACTTCTACTCCGGAAAGCGGTTCCGCTTCTCCAGGTTCTACCAGCGAAT |
| | | CTCCTTCTGGCACCGCTCCAGGTACCTCTCCTAGCGGCGAATCTTCTACCGCTCCAGG |
| | | TTCTACTAGCGAATCTCCTTCTGGCACTGCACCAGGTTCTACCAGCGAATCTCCTTCT |
| | | GGCACCGCTCCAGGTACCTCTCCTAGCGGCGAATCTTCTACCGCTCCAGGTTCTACTA |
| | | GCGAATCTCCTTCTGGCACTGCACCAGGTTCTACCAGCGAATCTCCTTCTGGCACCGC |
| | | TCCAGGTACCTCTCCTAGCGGCGAATCTTCTACCGCTCCAGGTTCTACTAGCGAATCT |
| | | CCTTCTGGCACTGCACCAGGTTCTACTAGCGAATCTCCTTCTGGCACTGCACCAGGTT |
| | | CTACCAGCGAATCTCCGTCTGGCACTGCACCAGGTACCTCTACCCCTGAAAGCGGTTC |
| | | CGCTTCTCCAGGTTCTACTAGCGAATCTCCTTCTGGTACCGCTCCAGGTACTTCTACC |
| | | CCTGAAAGCGGCTCCGCTTCTCCAGGTTCCACTAGCTCTACCGCTGAATCTCCGGGTC |
| | | CAGGTTCTACTAGCTCTACTGCAGAATCTCCTGGCCCAGGTACCTCTACTCCGGAAAG |
| | | CGGCTCTGCATCTCCAGGTACTTCTACCCCTGAAAGCGGTTCTGCATCTCCAGGTTCT |
| | | ACTAGCGAATCCCCGTCTGGTACCGCACCAGGTACTTCTACCCCGGAAAGCGGCTCTG |
| | | CTTCTCCAGGTACTTCTACCCCGGAAAGCGGCTCCGCATCTCCAGGTTCTACTAGCGA |
| | | ATCTCCTTCTGGTACCGCTCCAGGTTCTACCAGCGAATCCCCGTCTGGTACTGCTCCA |
| | | GGTTCTACCAGCGAATCTCCTTCTGGTACTGCACCAGGTTCTACTAGCTCTACTGCAG |
| | | AATCTCCTGGCCCAGGTACCTCTACTCCGGAAAGCGGCTCTGCATCTCCAGGTACTTC |
| | | TACCCCTGAAAGCGGTTCTGCATCTCCAGGTTCTACTAGCGAATCTCCTTCTGGCACT |
| | | GCACCAGGTTCTACCAGCGAATCTCCGTCTGGCACTGCACCAGGTACCTCTACCCCTG |
| | | AAAGCGGTTCCGCTTCTCCAGGTTCTACTAGCGAATCTCCTTCTGGCACTGCACCAGG |
| | | TTCTACCAGCGAATCTCCGTCTGGCACTGCACCAGGTACCTCTACCCCTGAAAGCGGT |
| | | TCCGCTTCTCCAGGTACTTCTCCGAGCGGTGAATCTTCTACCGCACCAGGTTCTACTA |
| | | GCTCTACCGCTGAATCTCCGGGCCCAGGTACTTCTCCGAGCGGTGAATCTTCTACTGC |
| | | TCCAGGTTCCACTAGCTCTACTGCTGAATCTCCTGGCCCAGGTACTTCTACTCCGGAA |
| | | AGCGGTTCCGCTTCTCCAGGTTCTACTAGCGAATCTCCGTCTGGCACCGCACCAGGTT |
| | | CTACTAGCTCTACTGCAGAATCTCCTGGCCCAGGTACCTCTACTCCGGAAAGCGGCTC |
| | | TGCATCTCCAGGTACTTCTACCCCTGAAAGCGGTTCTGCATCTCCA |
| AM875 | 252 | GGTACTTCTACTGAACCGTCTGAAGGCAGCGCACCAGGTAGCGAACCGGCTACTTCCG |
| | | GTTCTGAAACCCCAGGTAGCCCAGCAGGTTCTCCAACTTCTACTGAAGAAGGTTCTAC |
| | | CAGCTCTACCGCAGAATCTCCTGGTCCAGGTACCTCTACTCCGGAAAGCGGCTCTGCA |
| | | TCTCCAGGTTCTACTAGCGAATCTCCTTCTGGCACTGCACCAGGTTCTACTAGCGAAT |
| | | CCCCGTCTGGTACTGCTCCAGGTACTTCTACTCCTGAAAGCGGTTCCGCTTCTCCAGG |
| | | TACCTCTACTCCGGAAAGCGGTTCTGCATCTCCAGGTAGCGAACCGGCAACCTCCGGC |
| | | TCTGAAACCCCAGGTACCTCTGAAAGCGCTACTCCTGAATCCGGCCCAGGTAGCCCGG |
| | | CAGGTTCTCCGACTTCCACTGAGGAAGGTACCTCTACTGAACCTTCTGAGGGCAGCGC |
| | | TCCAGGTACTTCTGAAAGCGCTACCCCGGAGTCCGGTCCAGGTACTTCTACTGAACCG |
| | | TCCGAAGGTAGCGCACCAGGTACTTCTACCGAACCGTCCGAGGGTAGCGCACCAGGTA |
| | | GCCCAGCAGGTTCTCCTACCTCCACCGAGGAAGGTACTTCTACCGAACCGTCCGAGGG |
| | | TAGCGCACCAGGTACTTCTACCGAACCTTCCGAGGGCAGCGCACCAGGTACTTCTGAA |
| | | AGCGCTACCCCTGAGTCCGGCCCAGGTACTTCTGAAAGCGCTACTCCTGAATCCGGTC |
| | | CAGGTACCTCTACTGAACCTTCCGAAGGCAGCGCTCCAGGTACCTCTACCGAACCGTC |
| | | CGAGGGCAGCGCACCAGGTACTTCTGAAAGCGCAACCCCTGAATCCGGTCCAGGTACT |
| | | TCTACTGAACCTTCCGAAGGTAGCGCTCCAGGTAGCGAACCTGCTACTTCTGGTTCTG |
| | | AAACCCCAGGTAGCCCGGCTGGCTCTCCGACCTCCACCGAGGAAGGTAGCTCTACCCC |
| | | GTCTGGTGCTACTGGTTCTCCAGGTACTCCGGGCAGCGGTACTGCTTCTTCCTCTCCA |
| | | GGTAGCTCTACCCCTTCTGGTGCTACTGGCTCTCCAGGTACCTCTACCGAACCGTCCG |
| | | AGGGTAGCGCACCAGGTACCTCTACTGAACCGTCTGAGGGTAGCGCTCCAGGTAGCGA |
| | | ACCGGCAACCTCCGGTTCTGAAACTCCAGGTAGCCCTGCTGGCTCTCCGACTTCTACT |
| | | GAGGAAGGTAGCCCGGCTGGTTCTCCGACTTCTACTGAGGAAGGTACTTCTACCGAAC |
| | | CTTCCGAAGGTAGCGCTCCAGGTGCAAGCGCAAGCGGCGCGCCAAGCACGGGAGGTAC |
| | | TTCTGAAAGCGCTACTCCTGAGTCCGGCCCAGGTAGCCCGGCTGGCTCTCCGACTTCC |
| | | ACCGAGGAAGGTAGCCCGGCTGGCTCTCCAACTTCTACTGAAGAAGGTTCTACCAGCT |
| | | CTACCGCTGAATCTCCTGGCCCAGGTTCTACTAGCGAATCTCCGTCTGGCACCGCACC |
| | | AGGTACTTCCCCTAGCGGTGAATCTTCTACTGCACCAGGTACCCCTGGCAGCGGTACC |
| | | GCTTCTTCCTCTCCAGGTAGCTCTACCCCGTCTGGTGCTACTGGCTCTCCAGGTTCTA |
| | | GCCCGTCTGCATCTACCGGTACCGGCCCAGGTAGCGAACCGGCAACCTCCGGCTCTGA |
| | | AACTCCAGGTACTTCTGAAAGCGCTACTCCGGAATCCGGCCCAGGTAGCGAACCGGCT |
| | | ACTTCCGGCTCTGAAACCCCAGGTTCCACCAGCTCTACTGCAGAATCTCCGGGCCCAG |
| | | GTTCTACTAGCTCTACTGCAGAATCTCCGGGTCCAGGTACTTCTCCTAGCGGCGAATC |
| | | TTCTACCGCTCCAGGTAGCGAACCGGCAACCTCTGGCTCTGAAACTCCAGGTAGCGAA |
| | | CCTGCAACCTCCGGCTCTGAAACCCCAGGTACTTCTACTGAACCTTCTGAGGGCAGCG |
| | | CACCAGGTTCTACCAGCTCTACCGCAGAATCTCCTGGTCCAGGTACCTCTACTCCGGA |
| | | AAGCGGCTCTGCATCTCCAGGTTCTACTAGCGAATCTCCTTCTGGCACTGCACCAGGT |
| | | ACTTCTACCGAACCGTCCGAAGGCAGCGCTCCAGGTACCTCTACTGAACCTTCCGAGG |
| | | GCAGCGCTCCAGGTACCTCTACCGAACCTTCTGAAGGTAGCGCACCAGGTAGCTCTAC |
| | | TCCGTCTGGTGCAACCGGCTCCCCAGGTTCTAGCCCGTCTGCTTCCACTGGTACTGGC |
| | | CCAGGTGCTTCCCCGGGCACCAGCTCTACTGGTTCTCCAGGTAGCGAACCTGCTACCT |
| | | CCGGTTCTGAAACCCCAGGTACCTCTGAAAGCGCAACTCCGGAGTCTGGTCCAGGTAG |
| | | CCCTGCAGGTTCTCCTACCTCCACTGAGGAAGGTAGCTCTACTCCGTCTGGTGCAACC |
| | | GGCTCCCCAGGTTCTAGCCCGTCTGCTTCCACTGGTACTGGCCCAGGTGCTTCCCCGG |
| | | GCACCAGCTCTACTGGTTCTCCAGGTACCTCTGAAAGCGCTACTCCGGAGTCTGGCCC |
| | | AGGTACCTCTACTGAACCGTCTGAGGGTAGCGCTCCAGGTACTTCTACTGAACCGTCC |
| | | GAAGGTAGCGCACCA |
| AE864 | 253 | GGTAGCCCGGCTGGCTCTCCTACCTCTACTGAGGAAGGTACTTCTGAAAGCGCTACTC |
| | | CTGAGTCTGGTCCAGGTACCTCTACTGAACCGTCCGAAGGTAGCGCTCCAGGTAGCCC |
| | | AGCAGGCTCTCCGACTTCCACTGAGGAAGGTACTTCTACTGAACCTTCCGAAGGCAGC |
| | | GCACCAGGTACCTCTACTGAACCTTCTGAGGGCAGCGCTCCAGGTACTTCTGAAAGCG |
| | | CTACCCCGGAATCTGGCCCAGGTAGCGAACCGGCTACTTCTGGTTCTGAAACCCCAGG |
| | | TAGCGAACCGGCTACCTCCGGTTCTGAAACTCCAGGTAGCCCGGCAGGCTCTCCGACC |
| | | TCTACTGAGGAAGGTACTTCTGAAAGCGCAACCCCGGAGTCCGGCCCAGGTACCTCTA |
| | | CCGAACCGTCTGAGGGCAGCGCACCAGGTACTTCTACCGAACCGTCCGAGGGTAGCGC |
| | | ACCAGGTAGCCCAGCAGGTTCTCCTACCTCCACCGAGGAAGGTACTTCTACCGAACCG |
| | | TCCGAGGGTAGCGCACCAGGTACCTCTACTGAACCTTCTGAGGGCAGCGCTCCAGGTA |
| | | CTTCTGAAAGCGCTACCCCGGAGTCCGGTCCAGGTACTTCTACTGAACCGTCCGAAGG |
| | | TAGCGCACCAGGTACTTCTGAAAGCGCAACCCCTGAATCCGGTCCAGGTAGCGAACCG |
| | | GCTACTTCTGGCTCTGAGACTCCAGGTACTTCTACCGAACCGTCCGAAGGTAGCGCAC |
| | | CAGGTACTTCTACTGAACCGTCTGAAGGTAGCGCACCAGGTACTTCTGAAAGCGCAAC |
| | | CCCGGAATCCGGCCCAGGTACCTCTGAAAGCGCAACCCCGGAGTCCGGCCCAGGTAGC |
| | | CCTGCTGGCTCTCCAACCTCCACCGAAGAAGGTACCTCTGAAAGCGCAACCCCTGAAT |
| | | CCGGCCCAGGTAGCGAACCGGCAACCTCCGGTTCTGAAACCCCAGGTACCTCTGAAAG |
| | | CGCTACTCCGGAGTCTGGCCCAGGTACCTCTACTGAACCGTCTGAGGGTAGCGCTCCA |
| | | GGTACTTCTACTGAACCGTCCGAAGGTAGCGCACCAGGTACTTCTACCGAACCGTCCG |
| | | AAGGCAGCGCTCCAGGTACCTCTACTGAACCTTCCGAGGGCAGCGCTCCAGGTACCTC |
| | | TACCGAACCTTCTGAAGGTAGCGCACCAGGTACTTCTACCGAACCGTCCGAGGGTAGC |
| | | GCACCAGGTAGCCCAGCAGGTTCTCCTACCTCCACCGAGGAAGGTACTTCTACCGAAC |
| | | CGTCCGAGGGTAGCGCACCAGGTACCTCTGAAAGCGCAACTCCTGAGTCTGGCCCAGG |
| | | TAGCGAACCTGCTACCTCCGGCTCTGAGACTCCAGGTACCTCTGAAAGCGCAACCCCG |
| | | GAATCTGGTCCAGGTAGCGAACCTGCAACCTCTGGCTCTGAAACCCCAGGTACCTCTG |
| | | AAAGCGCTACTCCTGAATCTGGCCCAGGTACTTCTACTGAACCGTCCGAGGGCAGCGC |
| | | ACCAGGTACTTCTGAAAGCGCTACTCCTGAGTCCGGCCCAGGTAGCCCGGCTGGCTCT |
| | | CCGACTTCCACCGAGGAAGGTAGCCCGGCTGGCTCTCCAACTTCTACTGAAGAAGGTA |
| | | GCCCGGCAGGCTCTCCGACCTCTACTGAGGAAGGTACTTCTGAAAGCGCAACCCCGGA |
| | | GTCCGGCCCAGGTACCTCTACCGAACCGTCTGAGGGCAGCGCACCAGGTACCTCTGAA |
| | | AGCGCAACTCCTGAGTCTGGCCCAGGTAGCGAACCTGCTACCTCCGGCTCTGAGACTC |
| | | CAGGTACCTCTGAAAGCGCAACCCCGGAATCTGGTCCAGGTAGCGAACCTGCAACCTC |
| | | TGGCTCTGAAACCCCAGGTACCTCTGAAAGCGCTACTCCTGAATCTGGCCCAGGTACT |
| | | TCTACTGAACCGTCCGAGGGCAGCGCACCAGGTAGCCCTGCTGGCTCTCCAACCTCCA |
| | | CCGAAGAAGGTACCTCTGAAAGCGCAACCCCTGAATCCGGCCCAGGTAGCGAACCGGC |
| | | AACCTCCGGTTCTGAAACCCCAGGTACTTCTGAAAGCGCTACTCCTGAGTCCGGCCCA |
| | | GGTAGCCCGGCTGGCTCTCCGACTTCCACCGAGGAAGGTAGCCCGGCTGGCTCTCCAA |
| | | CTTCTACTGAAGAAGGTACTTCTACCGAACCTTCCGAGGGCAGCGCACCAGGTACTTC |
| | | TGAAAGCGCTACCCCTGAGTCCGGCCCAGGTACTTCTGAAAGCGCTACTCCTGAATCC |
| | | GGTCCAGGTACTTCTGAAAGCGCTACCCCGGAATCTGGCCCAGGTAGCGAACCGGCTA |
| | | CTTCTGGTTCTGAAACCCCAGGTAGCGAACCGGCTACCTCCGGTTCTGAAACTCCAGG |
| | | TAGCCCAGCAGGCTCTCCGACTTCCACTGAGGAAGGTACTTCTACTGAACCTTCCGAA |
| | | GGCAGCGCACCAGGTACCTCTACTGAACCTTCTGAGGGCAGCGCTCCAGGTAGCGAAC |
| | | CTGCAACCTCTGGCTCTGAAACCCCAGGTACCTCTGAAAGCGCTACTCCTGAATCTGG |
| | | CCCAGGTACTTCTACTGAACCGTCCGAGGGCAGCGCACCA |
| AF864 | 254 | GGTTCTACCAGCGAATCTCCTTCTGGCACCGCTCCAGGTACCTCTCCTAGCGGCGAAT |
| | | CTTCTACCGCTCCAGGTTCTACTAGCGAATCTCCTTCTGGCACTGCACCAGGTTCTAC |
| | | TAGCGAATCCCCGTCTGGTACTGCTCCAGGTACTTCTACTCCTGAAAGCGGTTCCGCT |
| | | TCTCCAGGTACCTCTACTCCGGAAAGCGGTTCTGCATCTCCAGGTTCTACCAGCGAAT |
| | | CTCCTTCTGGCACCGCTCCAGGTTCTACTAGCGAATCCCCGTCTGGTACCGCACCAGG |
| | | TACTTCTCCTAGCGGCGAATCTTCTACCGCACCAGGTTCTACTAGCGAATCTCCGTCT |
| | | GGCACTGCTCCAGGTACTTCTCCTAGCGGTGAATCTTCTACCGCTCCAGGTACTTCCC |
| | | CTAGCGGCGAATCTTCTACCGCTCCAGGTTCTACTAGCTCTACTGCAGAATCTCCGGG |
| | | CCCAGGTACCTCTCCTAGCGGTGAATCTTCTACCGCTCCAGGTACTTCTCCGAGCGGT |
| | | GAATCTTCTACCGCTCCAGGTTCTACTAGCTCTACTGCAGAATCTCCTGGCCCAGGTA |
| | | CCTCTACTCCGGAAAGCGGCTCTGCATCTCCAGGTACTTCTACCCCTGAAAGCGGTTC |
| | | TGCATCTCCAGGTTCTACTAGCGAATCTCCTTCTGGCACTGCACCAGGTTCTACCAGC |
| | | GAATCTCCGTCTGGCACTGCACCAGGTACCTCTACCCCTGAAAGCGGTTCCGCTTCTC |
| | | CAGGTTCTACCAGCTCTACCGCAGAATCTCCTGGTCCAGGTACCTCTACTCCGGAAAG |
| | | CGGCTCTGCATCTCCAGGTTCTACTAGCGAATCTCCTTCTGGCACTGCACCAGGTACT |
| | | TCTCCGAGCGGTGAATCTTCTACCGCACCAGGTTCTACTAGCTCTACCGCTGAATCTC |
| | | CGGGCCCAGGTACTTCTCCGAGCGGTGAATCTTCTACTGCTCCAGGTACCTCTACTCC |
| | | TGAAAGCGGTTCTGCATCTCCAGGTTCCACTAGCTCTACCGCAGAATCTCCGGGCCCA |
| | | GGTTCTACTAGCTCTACTGCTGAATCTCCTGGCCCAGGTTCTACTAGCTCTACTGCTG |
| | | AATCTCCGGGTCCAGGTTCTACCAGCTCTACTGCTGAATCTCCTGGTCCAGGTACCTC |
| | | CCCGAGCGGTGAATCTTCTACTGCACCAGGTTCTACTAGCGAATCTCCTTCTGGCACT |
| | | GCACCAGGTTCTACCAGCGAATCTCCGTCTGGCACTGCACCAGGTACCTCTACCCCTG |
| | | AAAGCGGTCCXXXXXXXXXXXXTGCAAGCGCAAGCGGCGCGCCAAGCACGGGAXXXXX |
| | | XXXTAGCGAATCTCCTTCTGGTACCGCTCCAGGTTCTACCAGCGAATCCCCGTCTGGT |
| | | ACTGCTCCAGGTTCTACCAGCGAATCTCCTTCTGGTACTGCACCAGGTTCTACTAGCG |
| | | AATCTCCTTCTGGTACCGCTCCAGGTTCTACCAGCGAATCCCCGTCTGGTACTGCTCC |
| | | AGGTTCTACCAGCGAATCTCCTTCTGGTACTGCACCAGGTACTTCTACTCCGGAAAGC |
| | | GGTTCCGCATCTCCAGGTACTTCTCCTAGCGGTGAATCTTCTACTGCTCCAGGTACCT |
| | | CTCCTAGCGGCGAATCTTCTACTGCTCCAGGTTCTACCAGCTCTACTGCTGAATCTCC |
| | | GGGTCCAGGTACTTCCCCGAGCGGTGAATCTTCTACTGCACCAGGTACTTCTACTCCG |
| | | GAAAGCGGTTCCGCTTCTCCAGGTTCTACCAGCGAATCTCCTTCTGGCACCGCTCCAG |
| | | GTTCTACTAGCGAATCCCCGTCTGGTACCGCACCAGGTACTTCTCCTAGCGGCGAATC |
| | | TTCTACCGCACCAGGTTCTACTAGCGAATCCCCGTCTGGTACCGCACCAGGTACTTCT |
| | | ACCCCGGAAAGCGGCTCTGCTTCTCCAGGTACTTCTACCCCGGAAAGCGGCTCCGCAT |
| | | CTCCAGGTTCTACTAGCGAATCTCCTTCTGGTACCGCTCCAGGTACTTCTACCCCTGA |
| | | AAGCGGCTCCGCTTCTCCAGGTTCCACTAGCTCTACCGCTGAATCTCCGGGTCCAGGT |
| | | TCTACCAGCGAATCTCCTTCTGGCACCGCTCCAGGTTCTACTAGCGAATCCCCGTCTG |
| | | GTACCGCACCAGGTACTTCTCCTAGCGGCGAATCTTCTACCGCACCAGGTTCTACCAG |
| | | CTCTACTGCTGAATCTCCGGGTCCAGGTACTTCCCCGAGCGGTGAATCTTCTACTGCA |
| | | CCAGGTACTTCTACTCCGGAAAGCGGTTCCGCTTCTCCAGGTACCTCCCCTAGCGGCG |
| | | AATCTTCTACTGCTCCAGGTACCTCTCCTAGCGGCGAATCTTCTACCGCTCCAGGTAC |
| | | CTCCCCTAGCGGTGAATCTTCTACCGCACCAGGTTCTACTAGCTCTACTGCTGAATCT |
| | | CCGGGTCCAGGTTCTACCAGCTCTACTGCTGAATCTCCTGGTCCAGGTACCTCCCCGA |
| | | GCGGTGAATCTTCTACTGCACCAGGTTCTAGCCCTTCTGCTTCCACCGGTACCGGCCC |
| | | AGGTAGCTCTACTCCGTCTGGTGCAACTGGCTCTCCAGGTAGCTCTACTCCGTCTGGT |
| | | GCAACCGGCTCCCCA |
| | | XXXX was inserted in two areas where no sequence |
| | | information is available. |
| AG864 | 255 | GGTGCTTCCCCGGGCACCAGCTCTACTGGTTCTCCAGGTTCTAGCCCGTCTGCTTCTA |
| | | CTGGTACTGGTCCAGGTTCTAGCCCTTCTGCTTCCACTGGTACTGGTCCAGGTACCCC |
| | | GGGTAGCGGTACCGCTTCTTCTTCTCCAGGTAGCTCTACTCCGTCTGGTGCTACCGGC |
| | | TCTCCAGGTTCTAACCCTTCTGCATCCACCGGTACCGGCCCAGGTGCTTCTCCGGGCA |
| | | CCAGCTCTACTGGTTCTCCAGGTACCCCGGGCAGCGGTACCGCATCTTCTTCTCCAGG |
| | | TAGCTCTACTCCTTCTGGTGCAACTGGTTCTCCAGGTACTCCTGGCAGCGGTACCGCT |
| | | TCTTCTTCTCCAGGTGCTTCTCCTGGTACTAGCTCTACTGGTTCTCCAGGTGCTTCTC |
| | | CGGGCACTAGCTCTACTGGTTCTCCAGGTACCCCGGGTAGCGGTACTGCTTCTTCCTC |
| | | TCCAGGTAGCTCTACCCCTTCTGGTGCAACCGGCTCTCCAGGTGCTTCTCCGGGCACC |
| | | AGCTCTACCGGTTCTCCAGGTACCCCGGGTAGCGGTACCGCTTCTTCTTCTCCAGGTA |
| | | GCTCTACTCCGTCTGGTGCTACCGGCTCTCCAGGTTCTAACCCTTCTGCATCCACCGG |
| | | TACCGGCCCAGGTTCTAGCCCTTCTGCTTCCACCGGTACTGGCCCAGGTAGCTCTACC |
| | | CCTTCTGGTGCTACCGGCTCCCCAGGTAGCTCTACTCCTTCTGGTGCAACTGGCTCTC |
| | | CAGGTGCATCTCCGGGCACTAGCTCTACTGGTTCTCCAGGTGCATCCCCTGGCACTAG |
| | | CTCTACTGGTTCTCCAGGTGCTTCTCCTGGTACCAGCTCTACTGGTTCTCCAGGTACT |
| | | CCTGGCAGCGGTACCGCTTCTTCTTCTCCAGGTGCTTCTCCTGGTACTAGCTCTACTG |
| | | GTTCTCCAGGTGCTTCTCCGGGCACTAGCTCTACTGGTTCTCCAGGTGCTTCCCCGGG |
| | | CACTAGCTCTACCGGTTCTCCAGGTTCTAGCCCTTCTGCATCTACTGGTACTGGCCCA |
| | | GGTACTCCGGGCAGCGGTACTGCTTCTTCCTCTCCAGGTGCATCTCCGGGCACTAGCT |
| | | CTACTGGTTCTCCAGGTGCATCCCCTGGCACTAGCTCTACTGGTTCTCCAGGTGCTTC |
| | | TCCTGGTACCAGCTCTACTGGTTCTCCAGGTAGCTCTACTCCGTCTGGTGCAACCGGT |
| | | TCCCCAGGTAGCTCTACTCCTTCTGGTGCTACTGGCTCCCCAGGTGCATCCCCTGGCA |
| | | CCAGCTCTACCGGTTCTCCAGGTACCCCGGGCAGCGGTACCGCATCTTCCTCTCCAGG |
| | | TAGCTCTACCCCGTCTGGTGCTACCGGTTCCCCAGGTAGCTCTACCCCGTCTGGTGCA |
| | | ACCGGCTCCCCAGGTAGCTCTACTCCGTCTGGTGCAACCGGCTCCCCAGGTTCTAGCC |
| | | CGTCTGCTTCCACTGGTACTGGCCCAGGTGCTTCCCCGGGCACCAGCTCTACTGGTTC |
| | | TCCAGGTGCATCCCCGGGTACCAGCTCTACCGGTTCTCCAGGTACTCCTGGCAGCGGT |
| | | ACTGCATCTTCCTCTCCAGGTGCTTCTCCGGGCACCAGCTCTACTGGTTCTCCAGGTG |
| | | CATCTCCGGGCACTAGCTCTACTGGTTCTCCAGGTGCATCCCCTGGCACTAGCTCTAC |
| | | TGGTTCTCCAGGTGCTTCTCCTGGTACCAGCTCTACTGGTTCTCCAGGTACCCCTGGT |
| | | AGCGGTACTGCTTCTTCCTCTCCAGGTAGCTCTACTCCGTCTGGTGCTACCGGTTCTC |
| | | CAGGTACCCCGGGTAGCGGTACCGCATCTTCTTCTCCAGGTAGCTCTACCCCGTCTGG |
| | | TGCTACTGGTTCTCCAGGTACTCCGGGCAGCGGTACTGCTTCTTCCTCTCCAGGTAGC |
| | | TCTACCCCTTCTGGTGCTACTGGCTCTCCAGGTAGCTCTACCCCGTCTGGTGCTACTG |
| | | GCTCCCCAGGTTCTAGCCCTTCTGCATCCACCGGTACCGGTCCAGGTTCTAGCCCGTC |
| | | TGCATCTACTGGTACTGGTCCAGGTGCATCCCCGGGCACTAGCTCTACCGGTTCTCCA |
| | | GGTACTCCTGGTAGCGGTACTGCTTCTTCTTCTCCAGGTAGCTCTACTCCTTCTGGTG |
| | | CTACTGGTTCTCCAGGTTCTAGCCCTTCTGCATCCACCGGTACCGGCCCAGGTTCTAG |
| | | CCCGTCTGCTTCTACCGGTACTGGTCCAGGTGCTTCTCCGGGTACTAGCTCTACTGGT |
| | | TCTCCAGGTGCATCTCCTGGTACTAGCTCTACTGGTTCTCCAGGTAGCTCTACTCCGT |
| | | CTGGTGCAACCGGCTCTCCAGGTTCTAGCCCTTCTGCATCTACCGGTACTGGTCCAGG |
| | | TGCATCCCCTGGTACCAGCTCTACCGGTTCTCCAGGTTCTAGCCCTTCTGCTTCTACC |
| | | GGTACCGGTCCAGGTACCCCTGGCAGCGGTACCGCATCTTCCTCTCCAGGTAGCTCTA |
| | | CTCCGTCTGGTGCAACCGGTTCCCCAGGTAGCTCTACTCCTTCTGGTGCTACTGGCTC |
| | | CCCAGGTGCATCCCCTGGCACCAGCTCTACCGGTTCTCCA |
| AM923 | 256 | ATGGCTGAACCTGCTGGCTCTCCAACCTCCACTGAGGAAGGTGCATCCCCGGGCACCA |
| | | GCTCTACCGGTTCTCCAGGTAGCTCTACCCCGTCTGGTGCTACCGGCTCTCCAGGTAG |
| | | CTCTACCCCGTCTGGTGCTACTGGCTCTCCAGGTACTTCTACTGAACCGTCTGAAGGC |
| | | AGCGCACCAGGTAGCGAACCGGCTACTTCCGGTTCTGAAACCCCAGGTAGCCCAGCAG |
| | | GTTCTCCAACTTCTACTGAAGAAGGTTCTACCAGCTCTACCGCAGAATCTCCTGGTCC |
| | | AGGTACCTCTACTCCGGAAAGCGGCTCTGCATCTCCAGGTTCTACTAGCGAATCTCCT |
| | | TCTGGCACTGCACCAGGTTCTACTAGCGAATCCCCGTCTGGTACTGCTCCAGGTACTT |
| | | CTACTCCTGAAAGCGGTTCCGCTTCTCCAGGTACCTCTACTCCGGAAAGCGGTTCTGC |
| | | ATCTCCAGGTAGCGAACCGGCAACCTCCGGCTCTGAAACCCCAGGTACCTCTGAAAGC |
| | | GCTACTCCTGAATCCGGCCCAGGTAGCCCGGCAGGTTCTCCGACTTCCACTGAGGAAG |
| | | GTACCTCTACTGAACCTTCTGAGGGCAGCGCTCCAGGTACTTCTGAAAGCGCTACCCC |
| | | GGAGTCCGGTCCAGGTACTTCTACTGAACCGTCCGAAGGTAGCGCACCAGGTACTTCT |
| | | ACCGAACCGTCCGAGGGTAGCGCACCAGGTAGCCCAGCAGGTTCTCCTACCTCCACCG |
| | | AGGAAGGTACTTCTACCGAACCGTCCGAGGGTAGCGCACCAGGTACTTCTACCGAACC |
| | | TTCCGAGGGCAGCGCACCAGGTACTTCTGAAAGCGCTACCCCTGAGTCCGGCCCAGGT |
| | | ACTTCTGAAAGCGCTACTCCTGAATCCGGTCCAGGTACCTCTACTGAACCTTCCGAAG |
| | | GCAGCGCTCCAGGTACCTCTACCGAACCGTCCGAGGGCAGCGCACCAGGTACTTCTGA |
| | | AAGCGCAACCCCTGAATCCGGTCCAGGTACTTCTACTGAACCTTCCGAAGGTAGCGCT |
| | | CCAGGTAGCGAACCTGCTACTTCTGGTTCTGAAACCCCAGGTAGCCCGGCTGGCTCTC |
| | | CGACCTCCACCGAGGAAGGTAGCTCTACCCCGTCTGGTGCTACTGGTTCTCCAGGTAC |
| | | TCCGGGCAGCGGTACTGCTTCTTCCTCTCCAGGTAGCTCTACCCCTTCTGGTGCTACT |
| | | GGCTCTCCAGGTACCTCTACCGAACCGTCCGAGGGTAGCGCACCAGGTACCTCTACTG |
| | | AACCGTCTGAGGGTAGCGCTCCAGGTAGCGAACCGGCAACCTCCGGTTCTGAAACTCC |
| | | AGGTAGCCCTGCTGGCTCTCCGACTTCTACTGAGGAAGGTAGCCCGGCTGGTTCTCCG |
| | | ACTTCTACTGAGGAAGGTACTTCTACCGAACCTTCCGAAGGTAGCGCTCCAGGTGCAA |
| | | GCGCAAGCGGCGCGCCAAGCACGGGAGGTACTTCTGAAAGCGCTACTCCTGAGTCCGG |
| | | CCCAGGTAGCCCGGCTGGCTCTCCGACTTCCACCGAGGAAGGTAGCCCGGCTGGCTCT |
| | | CCAACTTCTACTGAAGAAGGTTCTACCAGCTCTACCGCTGAATCTCCTGGCCCAGGTT |
| | | CTACTAGCGAATCTCCGTCTGGCACCGCACCAGGTACTTCCCCTAGCGGTGAATCTTC |
| | | TACTGCACCAGGTACCCCTGGCAGCGGTACCGCTTCTTCCTCTCCAGGTAGCTCTACC |
| | | CCGTCTGGTGCTACTGGCTCTCCAGGTTCTAGCCCGTCTGCATCTACCGGTACCGGCC |
| | | CAGGTAGCGAACCGGCAACCTCCGGCTCTGAAACTCCAGGTACTTCTGAAAGCGCTAC |
| | | TCCGGAATCCGGCCCAGGTAGCGAACCGGCTACTTCCGGCTCTGAAACCCCAGGTTCC |
| | | ACCAGCTCTACTGCAGAATCTCCGGGCCCAGGTTCTACTAGCTCTACTGCAGAATCTC |
| | | CGGGTCCAGGTACTTCTCCTAGCGGCGAATCTTCTACCGCTCCAGGTAGCGAACCGGC |
| | | AACCTCTGGCTCTGAAACTCCAGGTAGCGAACCTGCAACCTCCGGCTCTGAAACCCCA |
| | | GGTACTTCTACTGAACCTTCTGAGGGCAGCGCACCAGGTTCTACCAGCTCTACCGCAG |
| | | AATCTCCTGGTCCAGGTACCTCTACTCCGGAAAGCGGCTCTGCATCTCCAGGTTCTAC |
| | | TAGCGAATCTCCTTCTGGCACTGCACCAGGTACTTCTACCGAACCGTCCGAAGGCAGC |
| | | GCTCCAGGTACCTCTACTGAACCTTCCGAGGGCAGCGCTCCAGGTACCTCTACCGAAC |
| | | CTTCTGAAGGTAGCGCACCAGGTAGCTCTACTCCGTCTGGTGCAACCGGCTCCCCAGG |
| | | TTCTAGCCCGTCTGCTTCCACTGGTACTGGCCCAGGTGCTTCCCCGGGCACCAGCTCT |
| | | ACTGGTTCTCCAGGTAGCGAACCTGCTACCTCCGGTTCTGAAACCCCAGGTACCTCTG |
| | | AAAGCGCAACTCCGGAGTCTGGTCCAGGTAGCCCTGCAGGTTCTCCTACCTCCACTGA |
| | | GGAAGGTAGCTCTACTCCGTCTGGTGCAACCGGCTCCCCAGGTTCTAGCCCGTCTGCT |
| | | TCCACTGGTACTGGCCCAGGTGCTTCCCCGGGCACCAGCTCTACTGGTTCTCCAGGTA |
| | | CCTCTGAAAGCGCTACTCCGGAGTCTGGCCCAGGTACCTCTACTGAACCGTCTGAGGG |
| | | TAGCGCTCCAGGTACTTCTACTGAACCGTCCGAAGGTAGCGCACCA |
| AE912 | 257 | ATGGCTGAACCTGCTGGCTCTCCAACCTCCACTGAGGAAGGTACCCCGGGTAGCGGTA |
| | | CTGCTTCTTCCTCTCCAGGTAGCTCTACCCCTTCTGGTGCAACCGGCTCTCCAGGTGC |
| | | TTCTCCGGGCACCAGCTCTACCGGTTCTCCAGGTAGCCCGGCTGGCTCTCCTACCTCT |
| | | ACTGAGGAAGGTACTTCTGAAAGCGCTACTCCTGAGTCTGGTCCAGGTACCTCTACTG |
| | | AACCGTCCGAAGGTAGCGCTCCAGGTAGCCCAGCAGGCTCTCCGACTTCCACTGAGGA |
| | | AGGTACTTCTACTGAACCTTCCGAAGGCAGCGCACCAGGTACCTCTACTGAACCTTCT |
| | | GAGGGCAGCGCTCCAGGTACTTCTGAAAGCGCTACCCCGGAATCTGGCCCAGGTAGCG |
| | | AACCGGCTACTTCTGGTTCTGAAACCCCAGGTAGCGAACCGGCTACCTCCGGTTCTGA |
| | | AACTCCAGGTAGCCCGGCAGGCTCTCCGACCTCTACTGAGGAAGGTACTTCTGAAAGC |
| | | GCAACCCCGGAGTCCGGCCCAGGTACCTCTACCGAACCGTCTGAGGGCAGCGCACCAG |
| | | GTACTTCTACCGAACCGTCCGAGGGTAGCGCACCAGGTAGCCCAGCAGGTTCTCCTAC |
| | | CTCCACCGAGGAAGGTACTTCTACCGAACCGTCCGAGGGTAGCGCACCAGGTACCTCT |
| | | ACTGAACCTTCTGAGGGCAGCGCTCCAGGTACTTCTGAAAGCGCTACCCCGGAGTCCG |
| | | GTCCAGGTACTTCTACTGAACCGTCCGAAGGTAGCGCACCAGGTACTTCTGAAAGCGC |
| | | AACCCCTGAATCCGGTCCAGGTAGCGAACCGGCTACTTCTGGCTCTGAGACTCCAGGT |
| | | ACTTCTACCGAACCGTCCGAAGGTAGCGCACCAGGTACTTCTACTGAACCGTCTGAAG |
| | | GTAGCGCACCAGGTACTTCTGAAAGCGCAACCCCGGAATCCGGCCCAGGTACCTCTGA |
| | | AAGCGCAACCCCGGAGTCCGGCCCAGGTAGCCCTGCTGGCTCTCCAACCTCCACCGAA |
| | | GAAGGTACCTCTGAAAGCGCAACCCCTGAATCCGGCCCAGGTAGCGAACCGGCAACCT |
| | | CCGGTTCTGAAACCCCAGGTACCTCTGAAAGCGCTACTCCGGAGTCTGGCCCAGGTAC |
| | | CTCTACTGAACCGTCTGAGGGTAGCGCTCCAGGTACTTCTACTGAACCGTCCGAAGGT |
| | | AGCGCACCAGGTACTTCTACCGAACCGTCCGAAGGCAGCGCTCCAGGTACCTCTACTG |
| | | AACCTTCCGAGGGCAGCGCTCCAGGTACCTCTACCGAACCTTCTGAAGGTAGCGCACC |
| | | AGGTACTTCTACCGAACCGTCCGAGGGTAGCGCACCAGGTAGCCCAGCAGGTTCTCCT |
| | | ACCTCCACCGAGGAAGGTACTTCTACCGAACCGTCCGAGGGTAGCGCACCAGGTACCT |
| | | CTGAAAGCGCAACTCCTGAGTCTGGCCCAGGTAGCGAACCTGCTACCTCCGGCTCTGA |
| | | GACTCCAGGTACCTCTGAAAGCGCAACCCCGGAATCTGGTCCAGGTAGCGAACCTGCA |
| | | ACCTCTGGCTCTGAAACCCCAGGTACCTCTGAAAGCGCTACTCCTGAATCTGGCCCAG |
| | | GTACTTCTACTGAACCGTCCGAGGGCAGCGCACCAGGTACTTCTGAAAGCGCTACTCC |
| | | TGAGTCCGGCCCAGGTAGCCCGGCTGGCTCTCCGACTTCCACCGAGGAAGGTAGCCCG |
| | | GCTGGCTCTCCAACTTCTACTGAAGAAGGTAGCCCGGCAGGCTCTCCGACCTCTACTG |
| | | AGGAAGGTACTTCTGAAAGCGCAACCCCGGAGTCCGGCCCAGGTACCTCTACCGAACC |
| | | GTCTGAGGGCAGCGCACCAGGTACCTCTGAAAGCGCAACTCCTGAGTCTGGCCCAGGT |
| | | AGCGAACCTGCTACCTCCGGCTCTGAGACTCCAGGTACCTCTGAAAGCGCAACCCCGG |
| | | AATCTGGTCCAGGTAGCGAACCTGCAACCTCTGGCTCTGAAACCCCAGGTACCTCTGA |
| | | AAGCGCTACTCCTGAATCTGGCCCAGGTACTTCTACTGAACCGTCCGAGGGCAGCGCA |
| | | CCAGGTAGCCCTGCTGGCTCTCCAACCTCCACCGAAGAAGGTACCTCTGAAAGCGCAA |
| | | CCCCTGAATCCGGCCCAGGTAGCGAACCGGCAACCTCCGGTTCTGAAACCCCAGGTAC |
| | | TTCTGAAAGCGCTACTCCTGAGTCCGGCCCAGGTAGCCCGGCTGGCTCTCCGACTTCC |
| | | ACCGAGGAAGGTAGCCCGGCTGGCTCTCCAACTTCTACTGAAGAAGGTACTTCTACCG |
| | | AACCTTCCGAGGGCAGCGCACCAGGTACTTCTGAAAGCGCTACCCCTGAGTCCGGCCC |
| | | AGGTACTTCTGAAAGCGCTACTCCTGAATCCGGTCCAGGTACTTCTGAAAGCGCTACC |
| | | CCGGAATCTGGCCCAGGTAGCGAACCGGCTACTTCTGGTTCTGAAACCCCAGGTAGCG |
| | | AACCGGCTACCTCCGGTTCTGAAACTCCAGGTAGCCCAGCAGGCTCTCCGACTTCCAC |
| | | TGAGGAAGGTACTTCTACTGAACCTTCCGAAGGCAGCGCACCAGGTACCTCTACTGAA |
| | | CCTTCTGAGGGCAGCGCTCCAGGTAGCGAACCTGCAACCTCTGGCTCTGAAACCCCAG |
| | | GTACCTCTGAAAGCGCTACTCCTGAATCTGGCCCAGGTACTTCTACTGAACCGTCCGA |
| | | GGGCAGCGCACCA |
| AM1296 | 258 | GGTACTTCTACTGAACCGTCTGAAGGCAGCGCACCAGGTAGCGAACCGGCTACTTCCG |
| | | GTTCTGAAACCCCAGGTAGCCCAGCAGGTTCTCCAACTTCTACTGAAGAAGGTTCTAC |
| | | CAGCTCTACCGCAGAATCTCCTGGTCCAGGTACCTCTACTCCGGAAAGCGGCTCTGCA |
| | | TCTCCAGGTTCTACTAGCGAATCTCCTTCTGGCACTGCACCAGGTTCTACTAGCGAAT |
| | | CCCCGTCTGGTACTGCTCCAGGTACTTCTACTCCTGAAAGCGGTTCCGCTTCTCCAGG |
| | | TACCTCTACTCCGGAAAGCGGTTCTGCATCTCCAGGTAGCGAACCGGCAACCTCCGGC |
| | | TCTGAAACCCCAGGTACCTCTGAAAGCGCTACTCCTGAATCCGGCCCAGGTAGCCCGG |
| | | CAGGTTCTCCGACTTCCACTGAGGAAGGTACCTCTACTGAACCTTCTGAGGGCAGCGC |
| | | TCCAGGTACTTCTGAAAGCGCTACCCCGGAGTCCGGTCCAGGTACTTCTACTGAACCG |
| | | TCCGAAGGTAGCGCACCAGGTACTTCTACCGAACCGTCCGAGGGTAGCGCACCAGGTA |
| | | GCCCAGCAGGTTCTCCTACCTCCACCGAGGAAGGTACTTCTACCGAACCGTCCGAGGG |
| | | TAGCGCACCAGGTACTTCTACCGAACCTTCCGAGGGCAGCGCACCAGGTACTTCTGAA |
| | | AGCGCTACCCCTGAGTCCGGCCCAGGTACTTCTGAAAGCGCTACTCCTGAATCCGGTC |
| | | CAGGTACCTCTACTGAACCTTCCGAAGGCAGCGCTCCAGGTACCTCTACCGAACCGTC |
| | | CGAGGGCAGCGCACCAGGTACTTCTGAAAGCGCAACCCCTGAATCCGGTCCAGGTACT |
| | | TCTACTGAACCTTCCGAAGGTAGCGCTCCAGGTAGCGAACCTGCTACTTCTGGTTCTG |
| | | AAACCCCAGGTAGCCCGGCTGGCTCTCCGACCTCCACCGAGGAAGGTAGCTCTACCCC |
| | | GTCTGGTGCTACTGGTTCTCCAGGTACTCCGGGCAGCGGTACTGCTTCTTCCTCTCCA |
| | | GGTAGCTCTACCCCTTCTGGTGCTACTGGCTCTCCAGGTACCTCTACCGAACCGTCCG |
| | | AGGGTAGCGCACCAGGTACCTCTACTGAACCGTCTGAGGGTAGCGCTCCAGGTAGCGA |
| | | ACCGGCAACCTCCGGTTCTGAAACTCCAGGTAGCCCTGCTGGCTCTCCGACTTCTACT |
| | | GAGGAAGGTAGCCCGGCTGGTTCTCCGACTTCTACTGAGGAAGGTACTTCTACCGAAC |
| | | CTTCCGAAGGTAGCGCTCCAGGTCCAGAACCAACGGGGCCGGCCCCAAGCGGAGGTAG |
| | | CGAACCGGCAACCTCCGGCTCTGAAACCCCAGGTACCTCTGAAAGCGCTACTCCTGAA |
| | | TCCGGCCCAGGTAGCCCGGCAGGTTCTCCGACTTCCACTGAGGAAGGTACTTCTGAAA |
| | | GCGCTACTCCTGAGTCCGGCCCAGGTAGCCCGGCTGGCTCTCCGACTTCCACCGAGGA |
| | | AGGTAGCCCGGCTGGCTCTCCAACTTCTACTGAAGAAGGTACTTCTGAAAGCGCTACT |
| | | CCTGAGTCCGGCCCAGGTAGCCCGGCTGGCTCTCCGACTTCCACCGAGGAAGGTAGCC |
| | | CGGCTGGCTCTCCAACTTCTACTGAAGAAGGTTCTACCAGCTCTACCGCTGAATCTCC |
| | | TGGCCCAGGTTCTACTAGCGAATCTCCGTCTGGCACCGCACCAGGTACTTCCCCTAGC |
| | | GGTGAATCTTCTACTGCACCAGGTTCTACCAGCGAATCTCCTTCTGGCACCGCTCCAG |
| | | GTTCTACTAGCGAATCCCCGTCTGGTACCGCACCAGGTACTTCTCCTAGCGGCGAATC |
| | | TTCTACCGCACCAGGTACTTCTACCGAACCTTCCGAGGGCAGCGCACCAGGTACTTCT |
| | | GAAAGCGCTACCCCTGAGTCCGGCCCAGGTACTTCTGAAAGCGCTACTCCTGAATCCG |
| | | GTCCAGGTAGCGAACCGGCAACCTCTGGCTCTGAAACCCCAGGTACCTCTGAAAGCGC |
| | | TACTCCGGAATCTGGTCCAGGTACTTCTGAAAGCGCTACTCCGGAATCCGGTCCAGGT |
| | | ACCTCTACTGAACCTTCTGAGGGCAGCGCTCCAGGTACTTCTGAAAGCGCTACCCCGG |
| | | AGTCCGGTCCAGGTACTTCTACTGAACCGTCCGAAGGTAGCGCACCAGGTACCTCCCC |
| | | TAGCGGCGAATCTTCTACTGCTCCAGGTACCTCTCCTAGCGGCGAATCTTCTACCGCT |
| | | CCAGGTACCTCCCCTAGCGGTGAATCTTCTACCGCACCAGGTACTTCTACCGAACCGT |
| | | CCGAGGGTAGCGCACCAGGTAGCCCAGCAGGTTCTCCTACCTCCACCGAGGAAGGTAC |
| | | TTCTACCGAACCGTCCGAGGGTAGCGCACCAGGTTCTAGCCCTTCTGCTTCCACCGGT |
| | | ACCGGCCCAGGTAGCTCTACTCCGTCTGGTGCAACTGGCTCTCCAGGTAGCTCTACTC |
| | | CGTCTGGTGCAACCGGCTCCCCAGGTAGCTCTACCCCGTCTGGTGCTACCGGCTCTCC |
| | | AGGTAGCTCTACCCCGTCTGGTGCAACCGGCTCCCCAGGTGCATCCCCGGGTACTAGC |
| | | TCTACCGGTTCTCCAGGTGCAAGCGCAAGCGGCGCGCCAAGCACGGGAGGTACTTCTC |
| | | CGAGCGGTGAATCTTCTACCGCACCAGGTTCTACTAGCTCTACCGCTGAATCTCCGGG |
| | | CCCAGGTACTTCTCCGAGCGGTGAATCTTCTACTGCTCCAGGTACCTCTGAAAGCGCT |
| | | ACTCCGGAGTCTGGCCCAGGTACCTCTACTGAACCGTCTGAGGGTAGCGCTCCAGGTA |
| | | CTTCTACTGAACCGTCCGAAGGTAGCGCACCAGGTTCTAGCCCTTCTGCATCTACTGG |
| | | TACTGGCCCAGGTAGCTCTACTCCTTCTGGTGCTACCGGCTCTCCAGGTGCTTCTCCG |
| | | GGTACTAGCTCTACCGGTTCTCCAGGTACTTCTACTCCGGAAAGCGGTTCCGCATCTC |
| | | CAGGTACTTCTCCTAGCGGTGAATCTTCTACTGCTCCAGGTACCTCTCCTAGCGGCGA |
| | | ATCTTCTACTGCTCCAGGTACTTCTGAAAGCGCAACCCCTGAATCCGGTCCAGGTAGC |
| | | GAACCGGCTACTTCTGGCTCTGAGACTCCAGGTACTTCTACCGAACCGTCCGAAGGTA |
| | | GCGCACCAGGTTCTACCAGCGAATCCCCTTCTGGTACTGCTCCAGGTTCTACCAGCGA |
| | | ATCCCCTTCTGGCACCGCACCAGGTACTTCTACCCCTGAAAGCGGCTCCGCTTCTCCA |
| | | GGTAGCCCGGCAGGCTCTCCGACCTCTACTGAGGAAGGTACTTCTGAAAGCGCAACCC |
| | | CGGAGTCCGGCCCAGGTACCTCTACCGAACCGTCTGAGGGCAGCGCACCAGGTAGCCC |
| | | TGCTGGCTCTCCAACCTCCACCGAAGAAGGTACCTCTGAAAGCGCAACCCCTGAATCC |
| | | GGCCCAGGTAGCGAACCGGCAACCTCCGGTTCTGAAACCCCAGGTAGCTCTACCCCGT |
| | | CTGGTGCTACCGGTTCCCCAGGTGCTTCTCCTGGTACTAGCTCTACCGGTTCTCCAGG |
| | | TAGCTCTACCCCGTCTGGTGCTACTGGCTCTCCAGGTTCTACTAGCGAATCCCCGTCT |
| | | GGTACTGCTCCAGGTACTTCCCCTAGCGGTGAATCTTCTACTGCTCCAGGTTCTACCA |
| | | GCTCTACCGCAGAATCTCCGGGTCCAGGTAGCTCTACCCCTTCTGGTGCAACCGGCTC |
| | | TCCAGGTGCATCCCCGGGTACCAGCTCTACCGGTTCTCCAGGTACTCCGGGTAGCGGT |
| | | ACCGCTTCTTCCTCTCCAGGTAGCCCTGCTGGCTCTCCGACTTCTACTGAGGAAGGTA |
| | | GCCCGGCTGGTTCTCCGACTTCTACTGAGGAAGGTACTTCTACCGAACCTTCCGAAGG |
| | | TAGCGCTCCA |
| BC864 | 259 | GGTACTTCCACCGAACCATCCGAACCAGGTAGCGCAGGTACTTCCACCGAACCATCCG |
| | | AACCTGGCAGCGCAGGTAGCGAACCGGCAACCTCTGGTACTGAACCATCAGGTAGCGG |
| | | CGCATCCGAGCCTACCTCTACTGAACCAGGTAGCGAACCGGCTACCTCCGGTACTGAG |
| | | CCATCAGGTAGCGAACCGGCAACTTCCGGTACTGAACCATCAGGTAGCGAACCGGCAA |
| | | CTTCCGGCACTGAACCATCAGGTAGCGGTGCATCTGAGCCGACCTCTACTGAACCAGG |
| | | TACTTCTACTGAACCATCTGAGCCGGGCAGCGCAGGTAGCGAACCAGCTACTTCTGGC |
| | | ACTGAACCATCAGGTACTTCTACTGAACCATCCGAACCAGGTAGCGCAGGTAGCGAAC |
| | | CTGCTACCTCTGGTACTGAGCCATCAGGTAGCGAACCGGCTACCTCTGGTACTGAACC |
| | | ATCAGGTACTTCTACCGAACCATCCGAGCCTGGTAGCGCAGGTACTTCTACCGAACCA |
| | | TCCGAGCCAGGCAGCGCAGGTAGCGAACCGGCAACCTCTGGCACTGAGCCATCAGGTA |
| | | GCGAACCAGCAACTTCTGGTACTGAACCATCAGGTACTAGCGAGCCATCTACTTCCGA |
| | | ACCAGGTGCAGGTAGCGGCGCATCCGAACCTACTTCCACTGAACCAGGTACTAGCGAG |
| | | CCATCCACCTCTGAACCAGGTGCAGGTAGCGAACCGGCAACTTCCGGCACTGAACCAT |
| | | CAGGTAGCGAACCGGCTACCTCTGGTACTGAACCATCAGGTACTTCTACCGAACCATC |
| | | CGAGCCTGGTAGCGCAGGTACTTCTACCGAACCATCCGAGCCAGGCAGCGCAGGTAGC |
| | | GGTGCATCCGAGCCGACCTCTACTGAACCAGGTAGCGAACCAGCAACTTCTGGCACTG |
| | | AGCCATCAGGTAGCGAACCAGCTACCTCTGGTACTGAACCATCAGGTAGCGAACCGGC |
| | | TACTTCCGGCACTGAACCATCAGGTAGCGAACCAGCAACCTCCGGTACTGAACCATCA |
| | | GGTACTTCCACTGAACCATCCGAACCGGGTAGCGCAGGTAGCGAACCGGCAACTTCCG |
| | | GCACTGAACCATCAGGTAGCGGTGCATCTGAGCCGACCTCTACTGAACCAGGTACTTC |
| | | TACTGAACCATCTGAGCCGGGCAGCGCAGGTAGCGAACCTGCAACCTCCGGCACTGAG |
| | | CCATCAGGTAGCGGCGCATCTGAACCAACCTCTACTGAACCAGGTACTTCCACCGAAC |
| | | CATCTGAGCCAGGCAGCGCAGGTAGCGGCGCATCTGAACCAACCTCTACTGAACCAGG |
| | | TAGCGAACCAGCAACTTCTGGTACTGAACCATCAGGTAGCGGCGCATCTGAGCCTACT |
| | | TCCACTGAACCAGGTAGCGAACCGGCAACTTCCGGCACTGAACCATCAGGTAGCGGTG |
| | | CATCTGAGCCGACCTCTACTGAACCAGGTACTTCTACTGAACCATCTGAGCCGGGCAG |
| | | CGCAGGTAGCGAACCGGCAACTTCCGGCACTGAACCATCAGGTAGCGGTGCATCTGAG |
| | | CCGACCTCTACTGAACCAGGTACTTCTACTGAACCATCTGAGCCGGGCAGCGCAGGTA |
| | | GCGAACCAGCTACTTCTGGCACTGAACCATCAGGTACTTCTACTGAACCATCCGAACC |
| | | AGGTAGCGCAGGTAGCGAACCTGCTACCTCTGGTACTGAGCCATCAGGTACTTCTACT |
| | | GAACCATCCGAGCCGGGTAGCGCAGGTACTTCCACTGAACCATCTGAACCTGGTAGCG |
| | | CAGGTACTTCCACTGAACCATCCGAACCAGGTAGCGCAGGTACTTCTACTGAACCATC |
| | | CGAGCCGGGTAGCGCAGGTACTTCCACTGAACCATCTGAACCTGGTAGCGCAGGTACT |
| | | TCCACTGAACCATCCGAACCAGGTAGCGCAGGTACTAGCGAACCATCCACCTCCGAAC |
| | | CAGGCGCAGGTAGCGGTGCATCTGAACCGACTTCTACTGAACCAGGTACTTCCACTGA |
| | | ACCATCTGAGCCAGGTAGCGCAGGTACTTCCACCGAACCATCCGAACCAGGTAGCGCA |
| | | GGTACTTCCACCGAACCATCCGAACCTGGCAGCGCAGGTAGCGAACCGGCAACCTCTG |
| | | GTACTGAACCATCAGGTAGCGGTGCATCCGAGCCGACCTCTACTGAACCAGGTAGCGA |
| | | ACCAGCAACTTCTGGCACTGAGCCATCAGGTAGCGAACCAGCTACCTCTGGTACTGAA |
| | | CCATCAGGTAGCGAACCGGCAACCTCTGGCACTGAGCCATCAGGTAGCGAACCAGCAA |
| | | CTTCTGGTACTGAACCATCAGGTACTAGCGAGCCATCTACTTCCGAACCAGGTGCAGG |
| | | TAGCGAACCTGCAACCTCCGGCACTGAGCCATCAGGTAGCGGCGCATCTGAACCAACC |
| | | TCTACTGAACCAGGTACTTCCACCGAACCATCTGAGCCAGGCAGCGCAGGTAGCGAAC |
| | | CTGCAACCTCCGGCACTGAGCCATCAGGTAGCGGCGCATCTGAACCAACCTCTACTGA |
| | | ACCAGGTACTTCCACCGAACCATCTGAGCCAGGCAGCGCA |
| BD864 | 260 | GGTAGCGAAACTGCTACTTCCGGCTCTGAGACTGCAGGTACTAGTGAATCCGCAACTA |
| | | GCGAATCTGGCGCAGGTAGCACTGCAGGCTCTGAGACTTCCACTGAAGCAGGTACTAG |
| | | CGAGTCCGCAACCAGCGAATCCGGCGCAGGTAGCGAAACTGCTACCTCTGGCTCCGAG |
| | | ACTGCAGGTAGCGAAACTGCAACCTCTGGCTCTGAAACTGCAGGTACTTCCACTGAAG |
| | | CAAGTGAAGGCTCCGCATCAGGTACTTCCACCGAAGCAAGCGAAGGCTCCGCATCAGG |
| | | TACTAGTGAGTCCGCAACTAGCGAATCCGGTGCAGGTAGCGAAACCGCTACCTCTGGT |
| | | TCCGAAACTGCAGGTACTTCTACCGAGGCTAGCGAAGGTTCTGCATCAGGTAGCACTG |
| | | CTGGTTCCGAGACTTCTACTGAAGCAGGTACTAGCGAATCTGCTACTAGCGAATCCGG |
| | | CGCAGGTACTAGCGAATCCGCTACCAGCGAATCCGGCGCAGGTAGCGAAACTGCAACC |
| | | TCTGGTTCCGAGACTGCAGGTACTAGCGAGTCCGCTACTAGCGAATCTGGCGCAGGTA |
| | | CTTCCACTGAAGCTAGTGAAGGTTCTGCATCAGGTAGCGAAACTGCTACTTCTGGTTC |
| | | CGAAACTGCAGGTAGCGAAACCGCTACCTCTGGTTCCGAAACTGCAGGTACTTCTACC |
| | | GAGGCTAGCGAAGGTTCTGCATCAGGTAGCACTGCTGGTTCCGAGACTTCTACTGAAG |
| | | CAGGTACTAGCGAGTCCGCTACTAGCGAATCTGGCGCAGGTACTTCCACTGAAGCTAG |
| | | TGAAGGTTCTGCATCAGGTAGCGAAACTGCTACTTCTGGTTCCGAAACTGCAGGTAGC |
| | | ACTGCTGGCTCCGAGACTTCTACCGAAGCAGGTAGCACTGCAGGTTCCGAAACTTCCA |
| | | CTGAAGCAGGTAGCGAAACTGCTACCTCTGGCTCTGAGACTGCAGGTACTAGCGAATC |
| | | TGCTACTAGCGAATCCGGCGCAGGTACTAGCGAATCCGCTACCAGCGAATCCGGCGCA |
| | | GGTAGCGAAACTGCAACCTCTGGTTCCGAGACTGCAGGTACTAGCGAATCTGCTACTA |
| | | GCGAATCCGGCGCAGGTACTAGCGAATCCGCTACCAGCGAATCCGGCGCAGGTAGCGA |
| | | AACTGCAACCTCTGGTTCCGAGACTGCAGGTAGCGAAACCGCTACCTCTGGTTCCGAA |
| | | ACTGCAGGTACTTCTACCGAGGCTAGCGAAGGTTCTGCATCAGGTAGCACTGCTGGTT |
| | | CCGAGACTTCTACTGAAGCAGGTAGCGAAACTGCTACTTCCGGCTCTGAGACTGCAGG |
| | | TACTAGTGAATCCGCAACTAGCGAATCTGGCGCAGGTAGCACTGCAGGCTCTGAGACT |
| | | TCCACTGAAGCAGGTAGCACTGCTGGTTCCGAAACCTCTACCGAAGCAGGTAGCACTG |
| | | CAGGTTCTGAAACCTCCACTGAAGCAGGTACTTCCACTGAGGCTAGTGAAGGCTCTGC |
| | | ATCAGGTAGCACTGCTGGTTCCGAAACCTCTACCGAAGCAGGTAGCACTGCAGGTTCT |
| | | GAAACCTCCACTGAAGCAGGTACTTCCACTGAGGCTAGTGAAGGCTCTGCATCAGGTA |
| | | GCACTGCAGGTTCTGAGACTTCCACCGAAGCAGGTAGCGAAACTGCTACTTCTGGTTC |
| | | CGAAACTGCAGGTACTTCCACTGAAGCTAGTGAAGGTTCCGCATCAGGTACTAGTGAG |
| | | TCCGCAACCAGCGAATCCGGCGCAGGTAGCGAAACCGCAACCTCCGGTTCTGAAACTG |
| | | CAGGTACTAGCGAATCCGCAACCAGCGAATCTGGCGCAGGTACTAGTGAGTCCGCAAC |
| | | CAGCGAATCCGGCGCAGGTAGCGAAACCGCAACCTCCGGTTCTGAAACTGCAGGTACT |
| | | AGCGAATCCGCAACCAGCGAATCTGGCGCAGGTAGCGAAACTGCTACTTCCGGCTCTG |
| | | AGACTGCAGGTACTTCCACCGAAGCAAGCGAAGGTTCCGCATCAGGTACTTCCACCGA |
| | | GGCTAGTGAAGGCTCTGCATCAGGTAGCACTGCTGGCTCCGAGACTTCTACCGAAGCA |
| | | GGTAGCACTGCAGGTTCCGAAACTTCCACTGAAGCAGGTAGCGAAACTGCTACCTCTG |
| | | GCTCTGAGACTGCAGGTACTAGCGAATCTGCTACTAGCGAATCCGGCGCAGGTACTAG |
| | | CGAATCCGCTACCAGCGAATCCGGCGCAGGTAGCGAAACTGCAACCTCTGGTTCCGAG |
| | | ACTGCAGGTAGCGAAACTGCTACTTCCGGCTCCGAGACTGCAGGTAGCGAAACTGCTA |
| | | CTTCTGGCTCCGAAACTGCAGGTACTTCTACTGAGGCTAGTGAAGGTTCCGCATCAGG |
| | | TACTAGCGAGTCCGCAACCAGCGAATCCGGCGCAGGTAGCGAAACTGCTACCTCTGGC |
| | | TCCGAGACTGCAGGTAGCGAAACTGCAACCTCTGGCTCTGAAACTGCAGGTACTAGCG |
| | | AATCTGCTACTAGCGAATCCGGCGCAGGTACTAGCGAATCCGCTACCAGCGAATCCGG |
| | | CGCAGGTAGCGAAACTGCAACCTCTGGTTCCGAGACTGCA |

One may clone the library of XTEN-encoding genes into one or more expression vectors known in the art. To facilitate the identification of well-expressing library members, one can construct the library as fusion to a reporter protein. Non-limiting examples of suitable reporter genes are green fluorescent protein, luciferase, alkaline phosphatase, and beta-galactosidase. By screening, one can identify short XTEN sequences that can be expressed in high concentration in the host organism of choice. Subsequently, one can generate a library of random XTEN dimers and repeat the screen for high level of expression. Subsequently, one can screen the resulting constructs for a number of properties such as level of expression, protease stability, or binding to antiserum.

One aspect of the invention is to provide polynucleotide sequences encoding the components of the fusion protein wherein the creation of the sequence has undergone codon optimization. Of particular interest is codon optimization with the goal of improving expression of the polypeptide compositions and to improve the genetic stability of the encoding gene in the production hosts. For example, codon optimization is of particular importance for XTEN sequences that are rich in glycine or that have very repetitive amino acid sequences. Codon optimization can be performed using computer programs (Gustafsson, C., et al. (2004) Trends Biotechnol, 22: 346-53), some of which minimize ribosomal pausing (Coda Genomics Inc.). In one embodiment, one can perform codon optimization by constructing codon libraries where all members of the library encode the same amino acid sequence but where codon usage is varied. Such libraries can be screened for highly expressing and genetically stable members that are particularly suitable for the large-scale production of XTEN-containing products. When designing XTEN sequences one can consider a number of properties. One can minimize the repetitiveness in the encoding DNA sequences. In addition, one can avoid or minimize the use of codons that are rarely used by the production host (e.g. the AGG and AGA arginine codons and one leucine codon in E. coli). In the case of E. coli, two glycine codons, GGA and GGG, are rarely used in highly expressed proteins. Thus codon optimization of the gene encoding XTEN sequences can be very desirable. DNA sequences that have a high level of glycine tend to have a high GC content that can lead to instability or low expression levels. Thus, when possible, it is preferred to choose codons such that the GC-content of XTEN-encoding sequence is suitable for the production organism that will be used to manufacture the XTEN.

Optionally, the full-length XTEN-encoding gene may comprise one or more sequencing islands. In this context, sequencing islands are short-stretch sequences that are distinct from the XTEN library construct sequences and that include a restriction site not present or expected to be present in the full-length XTEN-encoding gene. In one embodiment, a sequencing island is the sequence 5′-AGGTGCAAGCGCAAGCGGCGCGCCAAGCACGGGAGGT-3′ (SEQ ID NO: 261). In another embodiment a sequencing island is the sequence

(SEQ ID NO: 262)
5′-AGGTCCAGAACCAACGGGGCCGGCCCCAAGCGGAGGT-3′.

As an alternative, one can construct codon libraries where all members of the library encode the same amino acid sequence but where codon usage is varied. Such libraries can be screened for highly expressing and genetically stable members that are particularly suitable for the large-scale production of XTEN-containing products.

Optionally, one can sequence clones in the library to eliminate isolates that contain undesirable sequences. The initial library of short XTEN sequences can allow some variation in amino acid sequence. For instance one can randomize some codons such that a number of hydrophilic amino acids can occur in a particular position.

During the process of iterative multimerization one can screen the resulting library members for other characteristics like solubility or protease resistance in addition to a screen for high-level expression.

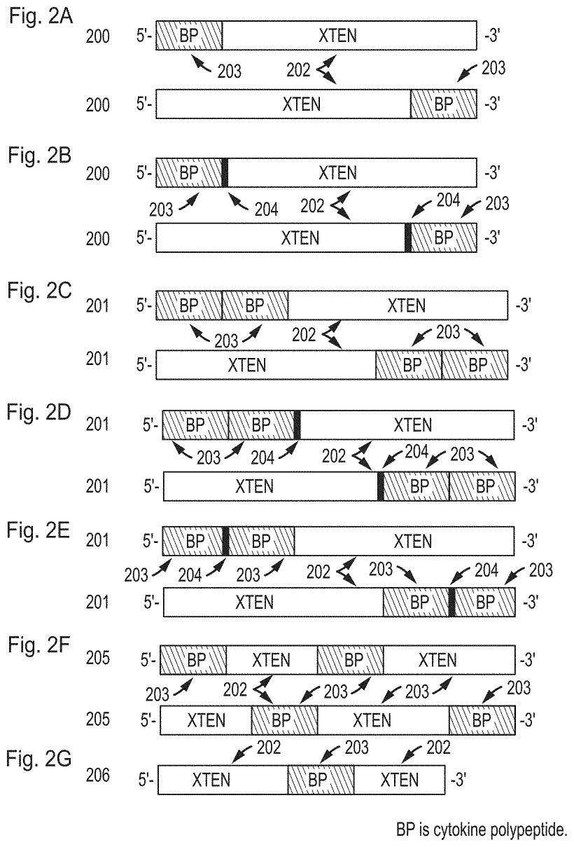

Once the gene that encodes the XTEN of desired length and properties is selected, it is genetically fused to the nucleotides encoding the N- and/or the C-terminus of the BP gene(s) by cloning it into the construct adjacent and in frame with the gene coding for BP or adjacent to a spacer sequence. The invention provides various permutations of the foregoing, depending on the BPXTEN to be encoded. For example, a gene encoding a BPXTEN fusion protein comprising two BP such as embodied by formula III or IV, as depicted above, the gene would have polynucleotides encoding two BP, at least a first XTEN, and optionally a second XTEN and/or spacer sequences. The step of cloning the BP genes into the XTEN construct can occur through a ligation or multimerization step. As shown in FIG. 2A-FIG. 2G, the constructs encoding BPXTEN fusion proteins can be designed in different configurations of the components XTEN 202, BP 203, and spacer sequences 204. In one embodiment, as illustrated in FIG. 2A, the construct comprises polynucleotide sequences complementary to, or those that encode a monomeric polypeptide of components in the following order (5′ to 3′) BP 203 and XTEN 202, or the reverse order. In another embodiment, as illustrated in FIG. 2B, the construct comprises polynucleotide sequences complementary to, or those that encode a monomeric polypeptide of components in the following order (5′ to 3′) BP 203, spacer sequence 204, and XTEN 202, or the reverse order. In another embodiment, as illustrated in FIG. 2C, the construct 201 encodes a monomeric BPXTEN comprising polynucleotide sequences complementary to, or those that encode components in the following order (5′ to 3′): two molecules of BP 203 and XTEN 202, or the reverse order. In another embodiment, as illustrated in FIG. 2D, the construct comprises polynucleotide sequences complementary to, or those that encode a monomeric polypeptide of components in the following order (5′ to 3′): two molecules of BP 203, spacer sequence 204, and XTEN 202, or the reverse order. In another embodiment, as illustrated in FIG. 2E, the construct comprises polynucleotide sequences complementary to, or those that encode a monomeric polypeptide of components in the following order (5′ to 3′): BP 203, spacer sequence 204, a second molecule of BP 203, and XTEN 202, or the reverse order. In another embodiment, as illustrated in FIG. 2F, the construct comprises polynucleotide sequences complementary to, or those that encode a monomeric polypeptide of components in the following order (5′ to 3′): BP 203, XTEN 202, BP 203, and a second XTEN 202, or the reverse sequence. The spacer polynucleotides can optionally comprise sequences encoding cleavage sequences. As will be apparent to those of skill in the art, other permutations of the foregoing are possible.

The invention also encompasses polynucleotides comprising XTEN-encoding polynucleotide variants that have a high percentage of sequence identity to (a) a polynucleotide sequence from Table 8, or (b) sequences that are complementary to the polynucleotides of (a). A polynucleotide with a high percentage of sequence identity is one that has at least about an 80% nucleic acid sequence identity, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, and alternatively at least about 99% nucleic acid sequence identity to (a) or (b) of the foregoing, or that can hybridize with the target polynucleotide or its complement under stringent conditions.

Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970. 48:443-453). When using a sequence alignment program such as BestFit, to determine the degree of sequence homology, similarity or identity, the default setting may be used, or an appropriate scoring matrix may be selected to optimize identity, similarity or homology scores.

Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the polynucleotides that encode the BPXTEN sequences under stringent conditions, such as those described herein.

The resulting polynucleotides encoding the BPXTEN chimeric compositions can then be individually cloned into an expression vector. The nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature.

Various vectors are publicly available. The vector may, for example, be in the form of a plasmid, cosmid, viral particle, or phage. Both expression and cloning vectors contain a nucleic acid sequence that enables the vector to replicate in one or more selected host cells. Such vector sequences are well known for a variety of bacteria, yeast, and viruses. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col E1, pCR1, pBR322, pMa1-C2, pET, pGEX as described by Smith, et al., Gene 57:31-40 (1988), pMB9 and derivatives thereof, plasmids such as RP4, phage DNAs such as the numerous derivatives of phage I such as NM98 9, as well as other phage DNA such as M13 and filamentous single stranded phage DNA; yeast plasmids such as the 2 micron plasmid or derivatives of the 2m plasmid, as well as centromeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like. The requirements are that the vectors are replicable and viable in the host cell of choice. Low- or high-copy number vectors may be used as desired.

Promoters suitable for use in expression vectors with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems can also contain a Shine-Dalgarno (S.D.) sequence operably linked to the DNA encoding BPXTEN polypeptides.

For example, in a baculovirus expression system, both non-fusion transfer vectors, such as, but not limited to pVL941 (BamHI cloning site, available from Summers, et al., Virology 84:390-402 (1978)), pVL1393 (BamHI, Sma1, Xba1, EcoRI, IVot1, Xma111, BgIII and Pst1 cloning sites; Invitrogen), pVL1392 (BgIII, Pst1, NotI, XmaIII, EcoRI, Xba11, Sma1 and BamHI cloning site; Summers, et al., Virology 84:390-402 (1978) and Invitrogen) and pBlueBacIII (BamHI, BgIII, Pst1, Nco1 and Hindi II cloning site, with blue/white recombinant screening, Invitrogen), and fusion transfer vectors such as, but not limited to, pAc7 00 (BamHI and Kpn1 cloning sites, in which the BamHI recognition site begins with the initiation codon; Summers, et al., Virology 84:390-402 (1978)), pAc701 and pAc70-2 (same as pAc700, with different reading frames), pAc360 [BamHI cloning site 36 base pairs downstream of a polyhedrin initiation codon; Invitrogen (1995)) and pBlueBacHisA, B, C (three different reading frames with BamH I, BgI II, Pst1, Nco 1 and Hind III cloning site, an N-terminal peptide for ProBond purification and blue/white recombinant screening of plaques; Invitrogen (220) can be used.

Mammalian expression vectors can comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements. Mammalian expression vectors contemplated for use in the invention include vectors with inducible promoters, such as the dihydrofolate reductase promoters, any expression vector with a DHFR expression cassette or a DHFR/methotrexate co-amplification vector such as pED (Pst1, Sai1, Sba1, Sma1 and EcoRI cloning sites, with the vector expressing both the cloned gene and DHFR; Randal J. Kaufman, 1991, Randal J. Kaufman, Current Protocols in Molecular Biology, 16, 12 (1991)). Alternatively a glutamine synthetase/methionine sulfoximine co-amplification vector, such as pEE14 (Hind111, Xba11, Sma1, Sba1, EcoRI and Sell cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI r SfH, Xho1, NotI, Nhe1, Hindi II, NheI, PvuII and Kpn1 cloning sites, constitutive RSV-LTR promoter, hygromycin selectable marker; Invitrogen), pCEP4 (BamHI, SfH, Xho1, NotI, Nhe1, Hind111, Nhe1, PvuII and Kpn1 cloning sites, constitutive hCMV immediate early gene promoter, hygromycin selectable marker; Invitrogen), pMEP4 (.Kpn1, Pvu1, Nhe1, Hind111, NotI, Xho1, Sfi1, BamHI cloning sites, inducible methallothionein H a gene promoter, hygromycin selectable marker, Invitrogen), pREP8 (BamHI, Xho1, NotI, Hind111, Nhe1 and Kpn1 cloning sites, RSV-LTR promoter, histidinol selectable marker; Invitrogen), pREP9 (Kpn1, Nhe1, Hind 111, NotI, Xho 1, Sfi 1, BamH I cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (Hind 111, BstXI, NotI, Sba1 and Apal cloning sites, G418 selection, Invitrogen), pRc/RSV (Hind II, Spel, BstXI, NotI, Xba1 cloning sites, G418 selection, Invitrogen) and the like. Vaccinia virus mammalian expression vectors (see, for example, Randall J. Kaufman, Current Protocols in Molecular Biology 16.12 (Frederick M. Ausubel, et al., eds. Wiley 1991) that can be used in the present invention include, but are not limited to, pSC1 1 (Sma1 cloning site, TK- and beta-gal selection), pMJ601 (Sal 1, Sma 1, A flI, Narl, BspMlI, BamHI, Apal, Nhe1, SacII, Kpn1 and Hind111 cloning sites; TK- and -gal selection), pTKgptFlS (EcoRI, Pst1, SaIII, Accl, HindII, Sba1, BamHI and Hpa cloning sites, TK or XPRT selection) and the like.

Yeast expression systems that can also be used in the present invention include, but are not limited to, the non-fusion pYES2 vector (XJbal, Sphl, Shol, NotI, GstXI, EcoRI, BstXI, BamHI, Sad, Kpn1 and Hind111 cloning sites, Invitrogen), the fusion pYESHisA, B, C (Xba11, Sphl, Shol, NotI, BstXI, EcoRI, BamHI, Sad, Kpn1 and Hindi II cloning sites, N-terminal peptide purified with ProBond resin and cleaved with enterokinase; Invitrogen), pRS vectors and the like.

In addition, the expression vector containing the chimeric BPXTEN fusion protein-encoding polynucleotide molecule may include drug selection markers. Such markers aid in cloning and in the selection or identification of vectors containing chimeric DNA molecules. For example, genes that confer resistance to neomycin, puromycin, hygromycin, dihydrofolate reductase (DHFR) inhibitor, guanine phosphoribosyl transferase (GPT), zeocin, and histidinol are useful selectable markers. Alternatively, enzymes such as herpes simplex virus thymidine kinase (tk) or chloramphenicol acetyltransferase (CAT) may be employed. Immunologic markers also can be employed. Any known selectable marker may be employed so long as it is capable of being expressed simultaneously with the nucleic acid encoding a gene product. Further examples of selectable markers are well known to one of skill in the art and include reporters such as enhanced green fluorescent protein (EGFP), beta-galactosidase (β-gal) or chloramphenicol acetyltransferase (CAT).

In one embodiment, the polynucleotide encoding a BPXTEN fusion protein composition can be fused C-terminally to an N-terminal signal sequence appropriate for the expression host system. Signal sequences are typically proteolytically removed from the protein during the translocation and secretion process, generating a defined N-terminus. A wide variety of signal sequences have been described for most expression systems, including bacterial, yeast, insect, and mammalian systems. A non-limiting list of preferred examples for each expression system follows herein. Preferred signal sequences are OmpA, PhoA, and DsbA for E. coli expression. Signal peptides preferred for yeast expression are ppL-alpha, DEX4, invertase signal peptide, acid phosphatase signal peptide, CPY, or INU1. For insect cell expression the preferred signal sequences are sexta adipokinetic hormone precursor, CP1, CP2, CP3, CP4, TPA, PAP, or gp67. For mammalian expression the preferred signal sequences are IL2L, SV40, IgG kappa and IgG lambda.

In another embodiment, a leader sequence, potentially comprising a well-expressed, independent protein domain, can be fused to the N-terminus of the BPXTEN sequence, separated by a protease cleavage site. While any leader peptide sequence which does not inhibit cleavage at the designed proteolytic site can be used, sequences in preferred embodiments will comprise stable, well-expressed sequences such that expression and folding of the overall composition is not significantly adversely affected, and preferably expression, solubility, and/or folding efficiency are significantly improved. A wide variety of suitable leader sequences have been described in the literature. A non-limiting list of suitable sequences includes maltose binding protein, cellulose binding domain, glutathione S-transferase, 6×His tag (SEQ ID NO: 263), FLAG tag, hemaglutinin tag, and green fluorescent protein. The leader sequence can also be further improved by codon optimization, especially in the second codon position following the ATG start codon, by methods well described in the literature and hereinabove.

Various in vitro enzymatic methods for cleaving proteins at specific sites are known. Such methods include use of enterokinase (DDDK (SEQ ID NO: 264)), Factor Xa (IDGR (SEQ ID NO: 265)), thrombin (LVPRGS (SEQ ID NO: 266)), PreScission™ (LEVLFQGP (SEQ ID NO: 267)), TEV protease (EQLYFQG (SEQ ID NO: 268)), 3C protease (ETLFQGP (SEQ ID NO: 269)), Sortase A (LPETG SEQ ID NO: 909), Granzyme B (D/X, N/X, M/N or S/X), inteins, SUMO, DAPase (TAGZyme™), Aeromonas aminopeptidase, Aminopeptidase M, and carboxypeptidases A and B. Additional methods are disclosed in Arnau, et al., Protein Expression and Purification 48: 1-13 (2006).

In other embodiments, an optimized polynucleotide sequence encoding at least about 20 to about 60 amino acids with XTEN characteristics can be included at the N-terminus of the XTEN sequence to promote the initiation of translation to allow for expression of XTEN fusions at the N-terminus of proteins without the presence of a helper domain. In an advantage of the foregoing, the sequence does not require subsequent cleavage, thereby reducing the number of steps to manufacture XTEN-containing compositions. As described in more detail in the Examples, the optimized N-terminal sequence has attributes of an unstructured protein, but may include nucleotide bases encoding amino acids selected for their ability to promote initiation of translation and enhanced expression. In one embodiment of the foregoing, the optimized polynucleotide encodes an XTEN sequence with at least about 90% sequence identity to AE912 (SEQ ID NO: 217). In another embodiment of the foregoing, the optimized polynucleotide encodes an XTEN sequence with at least about 90% sequence identity to AM923 (SEQ ID NO: 218).

In another embodiment, the protease site of the leader sequence construct is chosen such that it is recognized by an in vivo protease. In this embodiment, the protein is purified from the expression system while retaining the leader by avoiding contact with an appropriate protease. The full-length construct is then injected into a patient. Upon injection, the construct comes into contact with the protease specific for the cleavage site and is cleaved by the protease. In the case where the uncleaved protein is substantially less active than the cleaved form, this method has the beneficial effect of allowing higher initial doses while avoiding toxicity, as the active form is generated slowly in vivo. Some non-limiting examples of in vivo proteases which are useful for this application include tissue kallikrein, plasma kallikrein, trypsin, pepsin, chymotrypsin, thrombin, and matrix metalloproteinases, or the proteases of Table 5.

In this manner, a chimeric DNA molecule coding for a monomeric BPXTEN fusion protein is generated within the construct. Optionally, this chimeric DNA molecule may be transferred or cloned into another construct that is a more appropriate expression vector. At this point, a host cell capable of expressing the chimeric DNA molecule can be transformed with the chimeric DNA molecule. The vectors containing the DNA segments of interest can be transferred into the host cell by well-known methods, depending on the type of cellular host. For example, calcium chloride transfection is commonly utilized for prokaryotic cells, whereas calcium phosphate treatment, lipofection, or electroporation may be used for other cellular hosts. Other methods used to transform mammalian cells include the use of polybrene, protoplast fusion, liposomes, electroporation, and microinjection. See, generally, Sambrook, et al., supra.

The transformation may occur with or without the utilization of a carrier, such as an expression vector. Then, the transformed host cell is cultured under conditions suitable for expression of the chimeric DNA molecule encoding of BPXTEN.

The present invention also provides a host cell for expressing the monomeric fusion protein compositions disclosed herein. Examples of suitable eukaryotic host cells include, but are not limited to mammalian cells, such as VERO cells, HELA cells such as ATCC No. CCL2, CHO cell lines, COS cells, W138 cells, BHK cells, HepG2 cells, 3T3 cells, A549 cells, PC12 cells, K562 cells, 293 cells, Sf9 cells and CvI cells. Examples of suitable non-mammalian eukaryotic cells include eukaryotic microbes such as filamentous fungi or yeast are suitable cloning or expression hosts for encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus. Non-limiting examples of suitable prokaryotes include those from the genera: Actinoplanes; Archaeoglobus; Bdellovibrio; Borrelia; Chloroflexus; Enterococcus; Escherichia; Lactobacillus; Listeria; Oceanobacillus; Paracoccus; Pseudomonas; Staphylococcus; Streptococcus; Streptomyces; Thermoplasma; and Vibrio. Non-limiting examples of specific strains include: Archaeoglobus fulgidus; Bdellovibrio bacteriovorus; Borrelia burgdorferi; Chloroflexus aurantiacus; Enterococcus faecalis; Enterococcus faecium; Lactobacillus johnsonii; Lactobacillus plantarum; Lactococcus lactis; Listeria innocua; Listeria monocytogenes; Oceanobacillus iheyensis; Paracoccus zeaxanthinfaciens; Pseudomonas mevalonii; Staphylococcus aureus; Staphylococcus epidermidis; Staphylococcus haemolyticus; Streptococcus agalactiae; Streptomyces griseolosporeus; Streptococcus mutans; Streptococcus pneumoniae; Streptococcus pyogenes; Thermoplasma acidophilum; Thermoplasma volcanium; Vibrio cholerae; Vibrio parahaemolyticus; and Vibrio vulnificus.

Host cells containing the polynucleotides of interest can be cultured in conventional nutrient media (e.g., Ham's nutrient mixture) modified as appropriate for activating promoters, selecting transformants or amplifying genes. The culture conditions, such as temperature, pH and the like, are those previously used with the host cell selected for expression, and will be apparent to the ordinarily skilled artisan. Cells are typically harvested by centrifugation, disrupted by physical or chemical means, and the resulting crude extract retained for further purification. For compositions secreted by the host cells, supernatant from centrifugation is separated and retained for further purification. Microbial cells employed in expression of proteins can be disrupted by any convenient method, including freeze-thaw cycling, sonication, mechanical disruption, or use of cell lysing agents, all of which are well known to those skilled in the art. Embodiments that involve cell lysis may entail use of a buffer that contains protease inhibitors that limit degradation after expression of the chimeric DNA molecule. Suitable protease inhibitors include, but are not limited to leupeptin, pepstatin or aprotinin. The supernatant then may be precipitated in successively increasing concentrations of saturated ammonium sulfate.

Gene expression may be measured in a sample directly, for example, by conventional Southern blotting, Northern blotting to quantitate the transcription of mRNA ([Thomas, Proc. Natl. Acad. Sci. USA, 77:5201-5205 (1980)]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological of fluorescent methods, such as immunohistochemical staining of cells or tissue sections and assay of cell culture or body fluids or the detection of selectable markers, to directly quantitate the expression of gene product. Antibodies useful for immunohistochemical staining and/or assay of sample fluids may be either monoclonal or polyclonal, and may be prepared in any mammal. Conveniently, the antibodies may be prepared against a native sequence BP polypeptide or against a synthetic peptide based on the DNA sequences provided herein or against exogenous sequence fused to BP and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

Expressed BPXTEN polypeptide product(s) may be purified via methods known in the art or by methods disclosed herein. Procedures such as gel filtration, affinity purification, salt fractionation, ion exchange chromatography, size exclusion chromatography, hydroxyapatite adsorption chromatography, hydrophobic interaction chromatography and gel electrophoresis may be used; each tailored to recover and purify the fusion protein produced by the respective host cells. Some expressed BPXTEN may require refolding during isolation and purification. Methods of purification are described in Robert K. Scopes, Protein Purification: Principles and Practice, Charles R. Castor (ed.), Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994).

### Pharmaceutical Compositions

Cytokines can have utility in the treatment in various therapeutic or disease categories, including but not limited to cancer, rheumatoid arthritis, multiple sclerosis, myasthenia gravis, systemic lupus erythematosus, Alzheimer's disease, Schizophrenia, viral infections (e.g., chronic hepatitis C, AIDS), allergic asthma, retinal neurodegenerative processes, metabolic disorder, insulin resistance, and diabetic cardiomyopathy.

However, the therapeutic utility of cytokines can be limited in some situations because some of the cytokines such as IL-2, IL-12, IL15, Type I Interferons (alpha & beta), and IFN-gamma can be toxic to the host cells when delivered systematically. Extending the half-life of the circulating cytokine can be a way to reduce the cell toxicity by slowing the intracellular uptake.

The BPXTEN in the disclosure provides methods and compositions of extending the half-life of the cytokines by attachment of the cytokine to XTEN. In one embodiment, the pharmaceutical composition comprises the BPXTEN fusion protein and at least one pharmaceutically acceptable carrier. BPXTEN polypeptides of the present invention can be formulated according to known methods to prepare pharmaceutically useful compositions, whereby the polypeptide is combined in admixture with a pharmaceutically acceptable carrier vehicle, such as aqueous solutions or buffers, pharmaceutically acceptable suspensions and emulsions. Examples of non-aqueous solvents include propyl ethylene glycol, polyethylene glycol and vegetable oils. Therapeutic formulations are prepared for storage by mixing the active ingredient having the desired degree of purity with optional physiologically acceptable carriers, excipients or stabilizers, as described in Remington's Pharmaceutical Sciences 16th edition, Osol, A. Ed. (1980), in the form of lyophilized formulations or aqueous solutions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition, the pharmaceutical compositions can also contain other pharmaceutically active compounds or a plurality of compounds of the invention. The pharmaceutical composition can be formulated for oral, intradermal, subcutaneous, intravenous, intra-arterial, intraabdominal, intraperitoneal, intrathecal, or intramuscular administration. The pharmaceutical composition can be in a liquid form. The pharmaceutical composition can be in a pre-filled syringe for a single injection. The pharmaceutical composition can be formulated as a lyophilized powder to be reconstituted prior to administration.

More particularly, the present pharmaceutical compositions may be administered for therapy by any suitable route including oral, rectal, nasal, topical (including transdermal, aerosol, buccal and sublingual), vaginal, parenteral (including subcutaneous, subcutaneous by infusion pump, intramuscular, intravenous and intradermal), intravitreal, and pulmonary. It will also be appreciated that the preferred route will vary with the condition and age of the recipient, and the disease being treated.

In one embodiment, the pharmaceutical composition is administered subcutaneously. In this embodiment, the composition may be supplied as a lyophilized powder to be reconstituted prior to administration. The composition may also be supplied in a liquid form, which can be administered directly to a patient. In one embodiment, the composition is supplied as a liquid in a pre-filled syringe such that a patient can easily self-administer the composition.

Extended release formulations useful in the present invention may be oral formulations comprising a matrix and a coating composition. Suitable matrix materials may include waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm oil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition may comprise an insoluble matrix polymer and/or a water soluble material. Water soluble materials can be polymers such as polyethylene glycol, hydroxypropyl cellulose, hydroxypropyl methyl cellulose, polyvinylpyrrolidone, polyvinyl alcohol, or monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Optionally, an enteric polymer may be incorporated into the coating composition. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups. The coating composition may be plasticised by adding suitable plasticisers such as, for example, diethyl phthalate, citrate esters, polyethylene glycol, glycerol, acetylated glycerides, acetylated citrate esters, dibutylsebacate, and castor oil. The coating composition may also include a filler, which can be an insoluble material such as silicon dioxide, titanium dioxide, talc, kaolin, alumina, starch, powdered cellulose, MCC, or polacrilin potassium. The coating composition may be applied as a solution or latex in organic solvents or aqueous solvents or mixtures thereof. Solvents such as water, lower alcohol, lower chlorinated hydrocarbons, ketones, or mixtures thereof may be used.

The compositions of the invention may be formulated using a variety of excipients. Suitable excipients include microcrystalline cellulose (e.g. Avicel PH102, Avicel PH101), polymethacrylate, poly(ethyl acrylate, methyl methacrylate, trimethylammonioethyl methacrylate chloride) (such as Eudragit RS-30D), hydroxypropyl methylcellulose (Methocel K100M, Premium CR Methocel K100M, Methocel E5, Opadry®), magnesium stearate, talc, triethyl citrate, aqueous ethylcellulose dispersion (Surelease®), and protamine sulfate. The slow release agent may also comprise a carrier, which can comprise, for example, solvents, dispersion media, coatings, antibacterial and antifungal agents, isotonic and absorption delaying agents. Pharmaceutically acceptable salts can also be used in these slow release agents, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes may also be used as a carrier.

In another embodiment, the compositions of the present invention are encapsulated in liposomes, which have demonstrated utility in delivering beneficial active agents in a controlled manner over prolonged periods of time. Liposomes are closed bilayer membranes containing an entrapped aqueous volume. Liposomes may also be unilamellar vesicles possessing a single membrane bilayer or multilamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. In one embodiment, the liposome may be coated with a flexible water soluble polymer that avoids uptake by the organs of the mononuclear phagocyte system, primarily the liver and spleen. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose hydroxyethylcellulose, polyethyleneglycol, polyaspartamide and hydrophilic peptide sequences as described in U.S. Pat. Nos. 6,316,024; 6,126,966; 6,056,973; 6,043,094, the contents of which are incorporated by reference in their entirety.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phasphatidylglycerol, phosphatidylinositol, and sphingomyelin as disclosed in U.S. Pat. Nos. 6,056,973 and 5,874,104. The vesicle-forming lipids may also be glycolipids, cerebrosides, or cationic lipids, such as 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1 [(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N—(N′,N′-dimethylaminoethane) carbamoly] cholesterol (DC-Chol); or dimethyldioctadecylammonium (DDAB) also as disclosed in U.S. Pat. No. 6,056,973. Cholesterol may also be present in the proper range to impart stability to the vesicle as disclosed in U.S. Pat. Nos. 5,916,588 and 5,874,104.

Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479, the contents of which are incorporated herein by reference. These describe liposomes and lipid-coated microbubbles, and methods for their manufacture. Thus, one skilled in the art, considering both the disclosure of this invention and the disclosures of these other patents could produce a liposome for the extended release of the polypeptides of the present invention.

For liquid formulations, a desired property is that the formulation be supplied in a form that can pass through a 25, 28, 30, 31, 32 gauge needle for intravenous, intramuscular, intraarticular, or subcutaneous administration.

Administration via transdermal formulations can be performed using methods also known in the art, including those described generally in, e.g., U.S. Pat. Nos. 5,186,938 and 6,183,770, 4,861,800, 6,743,211, 6,945,952, 4,284,444, and WO 89/09051, incorporated herein by reference in their entireties. A transdermal patch is a particularly useful embodiment with polypeptides having absorption problems. Patches can be made to control the release of skin-permeable active ingredients over a 12 hour, 24 hour, 3 day, and 7 day period. In one example, a 2-fold daily excess of a polypeptide of the present invention is placed in a non-volatile fluid. The compositions of the invention are provided in the form of a viscous, non-volatile liquid. The penetration through skin of specific formulations may be measures by standard methods in the art (for example, Franz et al., J. Invest. Derm. 64:194-195 (1975)). Examples of suitable patches are passive transfer skin patches, iontophoretic skin patches, or patches with microneedles such as Nicoderm.

In other embodiments, the composition may be delivered via intranasal, buccal, or sublingual routes to the brain to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs. Preparation of a pharmaceutical composition for delivery in a subdermally implantable device can be performed using methods known in the art, such as those described in, e.g., U.S. Pat. Nos. 3,992,518; 5,660,848; and 5,756,115.

Osmotic pumps may be used as slow release agents in the form of tablets, pills, capsules or implantable devices. Osmotic pumps are well known in the art and readily available to one of ordinary skill in the art from companies experienced in providing osmotic pumps for extended release drug delivery. Examples are ALZA's DUROS™; ALZA's OROS™; Osmotica Pharmaceutical's Osmodex™ system; Shire Laboratories' EnSoTrol™ system; and Alzet™. Patents that describe osmotic pump technology are U.S. Pat. Nos. 6,890,918; 6,838,093; 6,814,979; 6,713,086; 6,534,090; 6,514,532; 6,361,796; 6,352,721; 6,294,201; 6,284,276; 6,110,498; 5,573,776; 4,200,0984; and 4,088,864, the contents of which are incorporated herein by reference. One skilled in the art, considering both the disclosure of this invention and the disclosures of these other patents could produce an osmotic pump for the extended release of the polypeptides of the present invention.

Syringe pumps may also be used as slow release agents. Such devices are described in U.S. Pat. Nos. 4,976,696; 4,933,185; 5,017,378; 6,309,370; 6,254,573; 4,435,173; 4,398,908; 6,572,585; 5,298,022; 5,176,502; 5,492,534; 5,318,540; and 4,988,337, the contents of which are incorporated herein by reference. One skilled in the art, considering both the disclosure of this invention and the disclosures of these other patents could produce a syringe pump for the extended release of the compositions of the present invention.

### Pharmaceutical Kits

In another aspect, the invention provides a kit to facilitate the use of the BPXTEN polypeptides. In one embodiment, the kit comprises, in at least a first container: (a) an amount of a BPXTEN fusion protein composition sufficient to treat a disease, condition or disorder upon administration to a subject in need thereof; and (b) an amount of a pharmaceutically acceptable carrier; together in a formulation ready for injection or for reconstitution with sterile water, buffer, or dextrose; together with a label identifying the BPXTEN drug and storage and handling conditions, and a sheet of the approved indications for the drug, instructions for the reconstitution and/or administration of the BPXTEN drug for the use for the prevention and/or treatment of an approved indication, appropriate dosage and safety information, and information identifying the lot and expiration of the drug. In another embodiment of the foregoing, the kit can comprise a second container that can carry a suitable diluent for the BPXTEN composition, which will provide the user with the appropriate concentration of BPXTEN to be delivered to the subject.

### EXAMPLES

### Example 1: Construction of XTEN

XTENs and various components can be made and assembled as described in WO 2010/091122, which is hereby incorporated by reference in its entirety and in particular with reference to its teachings regarding XTEN sequences and the manufacture and assembly thereof.

### Example 2: Methods of Producing and Evaluating BPXTEN; XTEN-Cytokine as Example

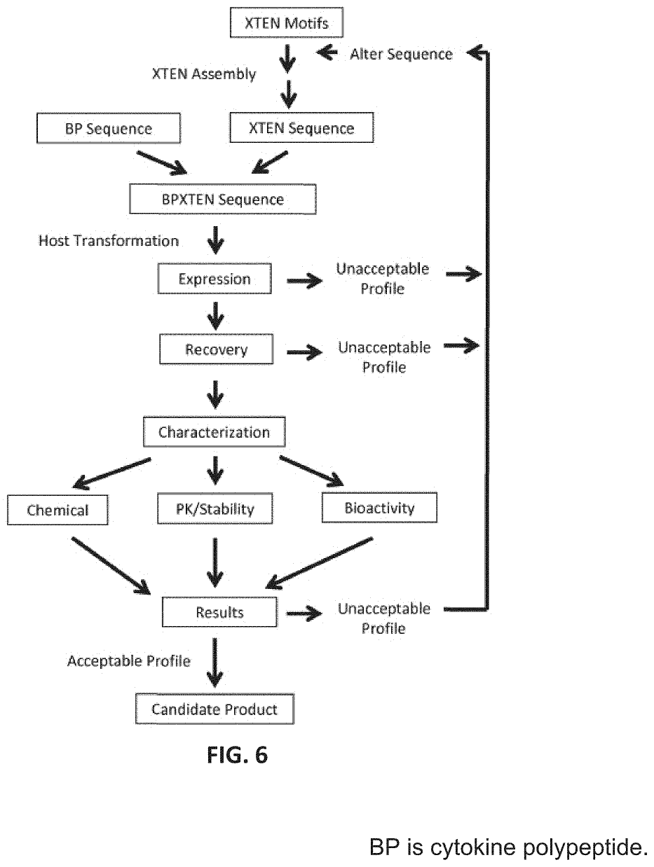

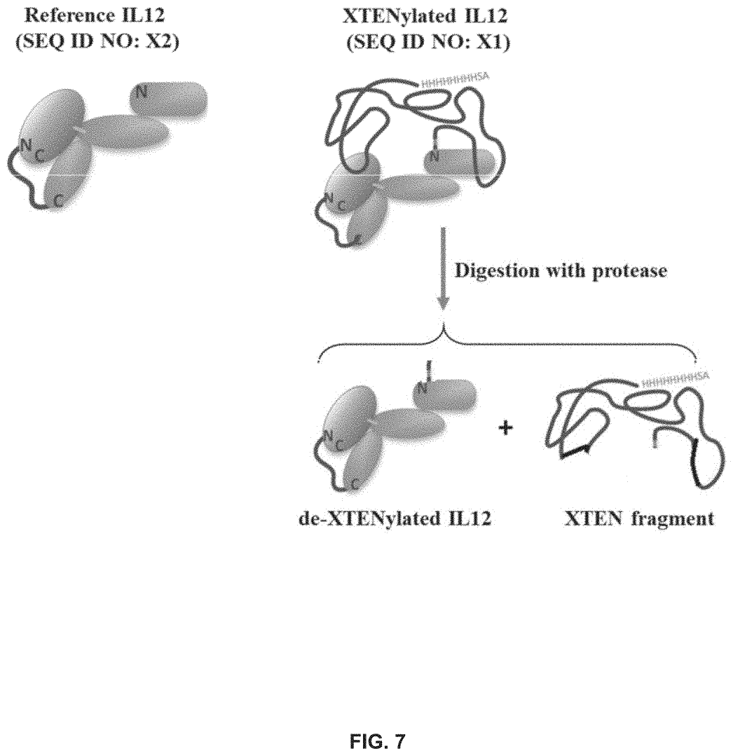

A general schema for producing and evaluating BPXTEN compositions is presented in FIG. 6, and forms the basis for the general description of this Example. Using the disclosed methods and those known to one of ordinary skill in the art, together with guidance provided in the illustrative examples, a skilled artesian can create and evaluate a range of BPXTEN fusion proteins comprising, XTENs, BP and variants of BP known in the art. The Example is, therefore, to be construed as merely illustrative, and not limitative of the methods in any way whatsoever; numerous variations will be apparent to the ordinarily skilled artisan. In this Prophetic Example, a BPXTEN of IL10 linked to an XTEN of the AE family of motifs would be created.

The general schema for producing polynucleotides encoding XTEN is presented in FIGS. 4 and 5. FIG. 5 is a schematic flowchart of representative steps in the assembly of a XTEN polynucleotide construct in one of the embodiments of the invention. Individual oligonucleotides 501 are annealed into sequence motifs 502 such as a 12 amino acid motif (“12-mer”), which is subsequently ligated with an oligo containing BbsI, and KpnI restriction sites 503. The motif libraries can be limited to specific sequence XTEN families; e.g., AD, AE, AF, AG, AM, or AQ sequences of Table 1. In this case, the motifs of the AE family (SEQ ID NOS: 186-189) would be used as the motif library, which are annealed to the 12-mer to create a “building block” length; e.g., a segment that encodes 36 amino acids. The gene encoding the XTEN sequence can be assembled by ligation and multimerization of the “building blocks” until the desired length of the XTEN gene 504 is achieved. As illustrated in FIG. 5, the XTEN length in this case is 48 amino acid residues, but longer lengths can be achieved by this process. For example, multimerization can be performed by ligation, overlap extension, PCR assembly or similar cloning techniques known in the art. The XTEN gene can be cloned into a stuffer vector. In the example illustrated in FIG. 5, the vector can encode a Flag sequence 506 followed by a stuffer sequence that is flanked by BsaI, BbsI, and KpnI sites 507 and a BP gene (e.g., exendin-4) 508, resulting in the gene encoding the BPXTEN 500, which, in this case encodes the fusion protein in the configuration, N- to C-terminus, XTEN-IL10.

DNA sequences encoding IL10 (or another candidate BP) can be conveniently obtained by standard procedures known in the art from a cDNA library prepared from an appropriate cellular source, from a genomic library, or may be created synthetically (e.g., automated nucleic acid synthesis) using DNA sequences obtained from publicly available databases, patents, or literature references. A gene or polynucleotide encoding the IL10 portion of the protein can then be cloned into a construct, such as those described herein, which can be a plasmid or other vector under control of appropriate transcription and translation sequences for high level protein expression in a biological system. A second gene or polynucleotide coding for the XTEN portion (in the case of FIG. 5 illustrated as an AE with 48 amino acid residues) can be genetically fused to the nucleotides encoding the N-terminus of the IL10 gene by cloning it into the construct adjacent and in frame with the gene coding for the IL10, through a ligation or multimerization step. In this manner, a chimeric DNA molecule coding for (or complementary to) the XTEN-IL10 BPXTEN fusion protein would be generated within the construct. The construct can be designed in different configurations to encode the various permutations of the fusion partners as a monomeric polypeptide. For example, the gene can be created to encode the fusion protein in the order (N- to C-terminus): IL10-XTEN; XTEN-IL10; IL10-XTEN-IL10; XTEN-IL10-XTEN; as well as multimers of the foregoing. Optionally, this chimeric DNA molecule may be transferred or cloned into another construct that is a more appropriate expression vector. At this point, a host cell capable of expressing the chimeric DNA molecule would be transformed with the chimeric DNA molecule. The vectors containing the DNA segments of interest can be transferred into an appropriate host cell by well-known methods, depending on the type of cellular host, as described supra.

Host cells containing the XTEN-IL10 expression vector would be cultured in conventional nutrient media modified as appropriate for activating the promoter. The culture conditions, such as temperature, pH and the like, are those previously used with the host cell selected for expression, and will be apparent to the ordinarily skilled artisan. After expression of the fusion protein, cells would be harvested by centrifugation, disrupted by physical or chemical means, and the resulting crude extract retained for purification of the fusion protein, as described below. For BPXTEN compositions secreted by the host cells, supernatant from centrifugation would be separated and retained for further purification.

Gene expression can be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, gene expression can be measured by immunological of fluorescent methods, such as immunohistochemical staining of cells to directly quantitate the expression of gene product. Antibodies useful for immunohistochemical staining and/or assay of sample fluids can be either monoclonal or polyclonal, and may be prepared in any mammal. Conveniently, the antibodies may be prepared against the IL10 sequence polypeptide using a synthetic peptide based on the sequences provided herein or against exogenous sequence fused to IL10 and encoding a specific antibody epitope. Examples of selectable markers are well known to one of skill in the art and include reporters such as enhanced green fluorescent protein (EGFP), beta-galactosidase (0-gal) or chloramphenicol acetyltransferase (CAT).

The XTEN-IL10 polypeptide product would be purified via methods known in the art. Procedures such as gel filtration, affinity purification, salt fractionation, ion exchange chromatography, size exclusion chromatography, hydroxyapatite adsorption chromatography, hydrophobic interaction chromatography or gel electrophoresis are all techniques that may be used in the purification. Specific methods of purification are described in Robert K. Scopes, Protein Purification: Principles and Practice, Charles R. Castor, ed., Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994).

As illustrated in FIG. 6, the isolated XTEN-IL10 fusion proteins would then be characterized for their chemical and activity properties. Isolated fusion protein would be characterized, e.g., for sequence, purity, apparent molecular weight, solubility and stability using standard methods known in the art. The fusion protein meeting expected standards would then be evaluated for activity, which can be measured in vitro or in vivo, using one or more assays disclosed herein.

In addition, the XTEN-IL10 fusion protein would be administered to one or more animal species to determine standard pharmacokinetic parameters, as described in Example 25.

By the iterative process of producing, expressing, and recovering XTEN-IL10 constructs, followed by their characterization using methods disclosed herein or others known in the art, the BPXTEN compositions comprising IL10 and an XTEN can be produced and evaluated by one of ordinary skill in the art to confirm the expected properties such as enhanced solubility, enhanced stability, improved pharmacokinetics and reduced immunogenicity, leading to an overall enhanced therapeutic activity compared to the corresponding unfused IL10. For those fusion proteins not possessing the desired properties, a different sequence can be constructed, expressed, isolated and evaluated by these methods in order to obtain a composition with such properties.

### Example 3: Analytical Size Exclusion Chromatography of XTEN Fusion Proteins

Size exclusion chromatography analysis is performed on fusion proteins containing various therapeutic proteins and unstructured recombinant proteins of increasing length. An exemplary assay uses a TSKGel-G4000 SWXL (7.8 mm×30 cm) column in which 40 μg of purified glucagon fusion protein at a concentration of 1 mg/ml is separated at a flow rate of 0.6 ml/min in 20 mM phosphate pH 6.8, 114 mM NaCl. Chromatogram profiles are monitored using OD214 nm and OD280 nm. Column calibration for all assays are performed using a size exclusion calibration standard from BioRad. It is thought that fusion proteins comprising IL10 and XTEN can reduce renal clearance, contributing to increased terminal half-life and improving the therapeutic or biologic effect relative to a corresponding un-fused biologically active protein.

### Example 4: Optimization of the Release Rate of C-Terminal XTEN

Variants of the fusion protein can be created in which the release rate of C-terminal XTEN is altered. As the rate of XTEN release by an XTEN release protease is dependent on the sequence of the XTEN release site, by varying the amino acid sequence in the XTEN release site one can control the rate of XTEN release. The sequence specificity of many proteases is well known in the art, and is documented in several databases. In this case, the amino acid specificity of proteases would be mapped using combinatorial libraries of substrates [Harris, J. L., et al. (2000) Proc Natl Acad Sci USA, 97: 7754] or by following the cleavage of substrate mixtures as illustrated in [Schellenberger, V., et al. (1993) Biochemistry, 32: 4344]. An alternative is the identification of desired protease cleavage sequences by phage display [Matthews, D., et al. (1993) Science, 260: 1113]. Constructs would be made with variant sequences and assayed for XTEN release using standard assays for detection of the XTEN polypeptides.

### Example 5: Analysis of Sequences for Secondary Structure by Prediction Algorithms

Amino acid sequences can be assessed for secondary structure via certain computer programs or algorithms, such as the well-known Chou-Fasman algorithm (Chou, P. Y., et al. (1974) Biochemistry, 13: 222-45) and the Garnier-Osguthorpe-Robson, or “GOR” method (Gamier J, Gibrat J F, Robson B. (1996). GOR method for predicting protein secondary structure from amino acid sequence. Methods Enzymol 266:540-553). For a given sequence, the algorithms can predict whether there exists some or no secondary structure at all, expressed as total and/or percentage of residues of the sequence that form, for example, alpha-helices or beta-sheets or the percentage of residues of the sequence predicted to result in random coil formation.

Several representative sequences from XTEN “families” have been assessed using two algorithm tools for the Chou-Fasman and GOR methods to assess the degree of secondary structure in these sequences. The Chou-Fasman tool was provided by William R. Pearson and the University of Virginia, at the “Biosupport” internet site, URL located on the World Wide Web at .fasta.bioch.virginia.edu/fasta_www2/fasta_www.cgi?rm=misc1 as it existed on Jun. 19, 2009. The GOR tool was provided by Pole Informatique Lyonnais at the Network Protein Sequence Analysis internet site, URL located on the World Wide Web at .npsa-pbil.ibcp.fr/cgi-bin/secpred_gor4.pl as it existed on Jun. 19, 2008.

As a first step in the analyses, a single XTEN sequence was analyzed by the two algorithms. The AE864 composition is a XTEN with 864 amino acid residues created from multiple copies of four 12 amino acid sequence motifs consisting of the amino acids G, S, T, E, P, and A. The sequence motifs are characterized by the fact that there is limited repetitiveness within the motifs and within the overall sequence in that the sequence of any two consecutive amino acids is not repeated more than twice in any one 12 amino acid motif, and that no three contiguous amino acids of full-length the XTEN are identical. Successively longer portions of the AF 864 sequence from the N-terminus were analyzed by the Chou-Fasman and GOR algorithms (the latter requires a minimum length of 17 amino acids). The sequences were analyzed by entering the FASTA format sequences into the prediction tools and running the analysis. The results from the analyses are presented in Table 10.

The results indicate that, by the Chou-Fasman calculations, the four motifs of the AE family (Table 1) have no alpha-helices or beta sheets. The sequence up to 288 residues was similarly found to have no alpha-helices or beta sheets. The 432 residue sequence is predicted to have a small amount of secondary structure, with only 2 amino acids contributing to an alpha-helix for an overall percentage of 0.5%. The full-length AF864 polypeptide has the same two amino acids contributing to an alpha-helix, for an overall percentage of 0.2%. Calculations for random coil formation revealed that with increasing length, the percentage of random coil formation increased. The first 24 amino acids of the sequence had 91% random coil formation, which increased with increasing length up to the 99.77% value for the full-length sequence.

Numerous XTEN sequences of 500 amino acids or longer from the other motif families were also analyzed and revealed that the majority had greater than 95% random coil formation. The exceptions were those sequences with one or more instances of three contiguous serine residues, which resulted in predicted beta-sheet formation. However, even these sequences still had approximately 99% random coil formation.

In contrast, a polypeptide sequence of 84 residues limited to A, S, and P amino acids was assessed by the Chou-Fasman algorithm, which predicted a high degree of predicted alpha-helices. The sequence, which had multiple repeat “AA” and “AAA” sequences, had an overall predicted percentage of alpha-helix structure of 69%. The GOR algorithm predicted 78.57% random coil formation; far less than any sequence consisting of 12 amino acid sequence motifs consisting of the amino acids G, S, T, E, P, analyzed in the present Example.

Conclusions: The analysis supports the conclusion that: 1) XTEN created from multiple sequence motifs of G, S, T, E, P, and A that have limited repetitiveness as to contiguous amino acids are predicted to have very low amounts of alpha-helices and beta-sheets; 2) that increasing the length of the XTEN does not appreciably increase the probability of alpha-helix or beta-sheet formation; and 3) that progressively increasing the length of the XTEN sequence by addition of non-repetitive 12-mers consisting of the amino acids G, 5, T, E, P, and A results in increased percentage of random coil formation. In contrast, polypeptides created from amino acids limited to A, S and P that have a higher degree of internal repetitiveness are predicted to have a high percentage of alpha-helices, as determined by the Chou-Fasman algorithm, as well as random coil formation. Based on the numerous sequences evaluated by these methods, it is generally the case that XTEN created from sequence motifs of G, S, T, E, P, and A that have limited repetitiveness (defined as no more than two identical contiguous amino acids in any one motif) greater than about 400 amino acid residues in length are expected to have very limited secondary structure. With the exception of motifs containing three contiguous serines, it is believed that any order or combination of sequence motifs from Table 1 can be used to create an XTEN polypeptide of a length greater than about 400 residues that will result in an XTEN sequence that is substantially devoid of secondary structure. Such sequences are expected to have the characteristics described in the BPXTEN embodiments of the invention disclosed herein.

TABLE 10
CHOU-FASMAN and GOR prediction calculations of polypeptide sequences

| SEQ | SEQ ID | | No. | Chou-Fasman | GOR |
|---|---|---|---|---|---|
| NAME | NO: | Sequence | Residues | Calculation | Calculation |
| | 289 | GSTSESPSGTAP | 12 | Residue totals*: H: 0 E: 0 | Not |
| | | | | percent: H: 0.0 E: 0.0 | Determined |
| | 290 | GTSTPESGSASP | 12 | Residue totals: H: 0 E: 0 | Not |
| | | | | percent: H: 0.0 E: 0.0 | Determined |
| | 291 | GTSPSGESSTAP | 12 | Residue totals: H: 0 E: 0 | Not |
| | | | | percent: H: 0.0 E: 0.0 | Determined |
| | 292 | GSTSSTAESPGP | 12 | Residue totals: H: 0 E: 0 | Not |
| | | | | percent: H: 0.0 E: 0.0 | Determined |
| | 293 | GSPAGSPTSTEEGTSESATPESGP | 24 | Residue totals: H: 0 E: 0 | 91.67% |
| | | | | percent: H: 0.0 E: 0.0 | |
| | 294 | GSPAGSPTSTEEGTSESATPESGPGT | 36 | Residue totals: H: 0 E: 0 | 94.44% |
| | | STEPSEGSAP | | percent: H: 0.0 E: 0.0 | |
| | 295 | GSPAGSPTSTEEGTSESATPESGPGT | 48 | Residue totals: H: 0 E: 0 | 93.75% |
| | | STEPSEGSAPGSPAGSPTSTEE | | percent: H: 0.0 E: 0.0 | |
| | 296 | GSPAGSPTSTEEGTSESATPESGPGT | 60 | Residue totals: H: 0 E: 0 | 96.67% |
| | | STEPSEGSAPGSPAGSPTSTEEGTST | | percent: H: 0.0 E: 0.0 | |
| | | EPSEGSAP | | | |
| | 297 | GSPAGSPTSTEEGTSESATPESGPGT | 108 | Residue totals: H: 0 E: 0 | 97.22% |
| | | STEPSEGSAPGSPAGSPTSTEEGTST | | percent: H: 0.0 E: 0.0 | |
| | | EPSEGSAPGTSTEPSEGSAPGTSESA | | | |
| | | TPESGPGSEPATSGSE | | | |
| | | TPGSEPATSGSETP | | | |
| | 298 | GSPAGSPTSTEEGTSESATPESGPGT | 216 | Residue totals: H: 0 E: 0 | 99.07% |
| | | STEPSEGSAPGSPAGSPTSTEEGTST | | percent: H: 0.0 E: 0.0 | |
| | | EPSEGSAPGTSTEPSEGSAPGTSESA | | | |
| | | TPESGPGSEPATSGSETPGSEPATSG | | | |
| | | SETPGSPAGSPTSTEEGTSESATPES | | | |
| | | GPGTSTEPSEGSAPGTSTEPSEGSAP | | | |
| | | GSPAGSPTSTEEGTSTEPSEGSAPGT | | | |
| | | STEPSEGSAPGTSESATPESGPGTST | | | |
| | | EPSEGSAP | | | |
| | 299 | GSPAGSPTSTEEGTSESATPESGPGT | 432 | Residue totals: H: 2 E: 3 | 99.54% |
| | | STEPSEGSAPGSPAGSPTSTEEGTST | | percent: H: 0.5 E : 0.7 | |
| | | EPSEGSAPGTSTEPSEGSAPGTSESA | | | |
| | | TPESGPGSEPATSGSETPGSEPATSG | | | |
| | | SETPGSPAGSPTSTEEGTSESATPES | | | |
| | | GPGTSTEPSEGSAPGTSTEPSEGSAP | | | |
| | | GSPAGSPTSTEEGTSTEPSEGSAPGT | | | |
| | | STEPSEGSAPGTSESATPESGPGTST | | | |
| | | EPSEGSAPGTSESATPESGPGSEPAT | | | |
| | | SGSETPGTSTEPSEGSAPGTSTEPSE | | | |
| | | GSAPGTSESATPESGPGTSESATPES | | | |
| | | GPGSPAGSPTSTEEGTSESATPESGP | | | |
| | | GSEPATSGSETPGTSESATPESGPGT | | | |
| | | STEPSEGSAPGTSTEPSEGSAPGTST | | | |
| | | EPSEGSAPGTSTEPSEGSAPGTSTEP | | | |
| | | SEGSAPGTSTEPSEGSAPGSPAGSPT | | | |
| | | STEEGTSTEPSEGSAP | | | |
| AE864 | 300 | GSPAGSPTSTEEGTSESATPESGPGT | 864 | Residue totals: H: 2 E: 3 | 99.77% |
| | | STEPSEGSAPGSPAGSPTSTEEGTST | | percent: H: 0.2 E: 0.3 | |
| | | EPSEGSAPGTSTEPSEGSAPGTSESA | | | |
| | | TPESGPGSEPATSGSETPGSEPATSG | | | |
| | | SETPGSPAGSPTSTEEGTSESATPES | | | |
| | | GPGTSTEPSEGSAPGTSTEPSEGSAP | | | |
| | | GSPAGSPTSTEEGTSTEPSEGSAPGT | | | |
| | | STEPSEGSAPGTSESATPESGPGTST | | | |
| | | EPSEGSAPGTSESATPESGPGSEPAT | | | |
| | | SGSETPGTSTEPSEGSAPGTSTEPSE | | | |
| | | GSAPGTSESATPESGPGTSESATPES | | | |
| | | GPGSPAGSPTSTEEGTSESATPESGP | | | |
| | | GSEPATSGSETPGTSESATPESGPGT | | | |
| | | STEPSEGSAPGTSTEPSEGSAPGTST | | | |
| | | EPSEGSAPGTSTEPSEGSAPGTSTEP | | | |
| | | SEGSAPGTSTEPSEGSAPGSPAGSPT | | | |
| | | STEEGTSTEPSEGSAPGTSESATPES | | | |
| | | GPGSEPATSGSETPGTSESATPESGP | | | |
| | | GSEPATSGSETPGTSESATPESGPGT | | | |
| | | STEPSEGSAPGTSESATPESGPGSPA | | | |
| | | GSPTSTEEGSPAGSPTSTEEGSPAGS | | | |
| | | PTSTEEGTSESATPESGPGTSTEPSE | | | |
| | | GSAPGTSESATPESGPGSEPATSGSE | | | |
| | | TPGTSESATPESGPGSEPATSGSETP | | | |
| | | GTSESATPESGPGTSTEPSEGSAPGS | | | |
| | | PAGSPTSTEEGTSESATPESGPGSEP | | | |
| | | ATSGSETPGTSESATPESGPGSPAGS | | | |
| | | PTSTEEGSPAGSPTSTEEGTSTEPSE | | | |
| | | GSAPGTSESATPESGPGTSESATPES | | | |
| | | GPGTSESATPESGPGSEPATSGSETP | | | |
| | | GSEPATSGSETPGSPAGSPTSTEEGT | | | |
| | | STEPSEGSAPGTSTEPSEGSAPGSEP | | | |
| | | ATSGSETPGTSESATPESGPGTSTEP | | | |
| | | SEGSAP | | | |
| AD 576 | 301 | GSSESGSSEGGPGSGGEPSESGSSGS | 576 | Residue totals: H: 7 E: 0 | 99.65% |
| | | SESGSSEGGPGSSESGSSEGGPGSSE | | percent: H: 1.2 E: 0.0 | |
| | | SGSSEGGPGSSESGSSEGGPGSSESG | | | |
| | | SSEGGPGESPGGSSGSESGSEGSSGP | | | |
| | | GESSGSSESGSSEGGPGSSESGSSEG | | | |
| | | GPGSSESGSSEGGPGSGGEPSESGSS | | | |
| | | GESPGGSSGSESGESPGGSSGSESGS | | | |
| | | GGEPSESGSSGSSESGSSEGGPGSGG | | | |
| | | EPSESGSSGSGGEPSESGSSGSEGSS | | | |
| | | GPGESSGESPGGSSGSESGSGGEPSE | | | |
| | | SGSSGSGGEPSESGSSGSGGEPSESG | | | |
| | | SSGSSESGSSEGGPGESPGGSSGSES | | | |
| | | GESPGGSSGSESGESPGGSSGSESGE | | | |
| | | SPGGSSGSESGESPGGSSGSESGSSE | | | |
| | | SGSSEGGPGSGGEPSESGSSGSEGSS | | | |
| | | GPGESSGSSESGSSEGGPGSGGEPSE | | | |
| | | SGSSGSSESGSSEGGPGSGGEPSESG | | | |
| | | SSGESPGGSSGSESGESPGGSSGSES | | | |
| | | GSSESGSSEGGPGSGGEPSESGSSGS | | | |
| | | SESGSSEGGPGSGGEPSESGSSGSGG | | | |
| | | EPSESGSSGESPGGSSGSESGSEGSS | | | |
| | | GPGESSGSSESGSSEGGPGSEGSSGP | | | |
| | | GESS | | | |
| AE576 | 302 | GSPAGSPTSTEEGTSESATPESGPGT | 576 | Residue totals: H: 2 E: 0 | 99.65% |
| | | STEPSEGSAPGSPAGSPTSTEEGTST | | percent: H: 0.4 E: 0.0 | |
| | | EPSEGSAPGTSTEPSEGSAPGTSESA | | | |
| | | TRESGPGSEPATSGSETPGSEPATSG | | | |
| | | SETPGSPAGSPTSTEEGTSESATPES | | | |
| | | GPGTSTEPSEGSAPGTSTEPSEGSAP | | | |
| | | GSPAGSPTSTEEGTSTEPSEGSAPGT | | | |
| | | STEPSEGSAPGTSESATPESGEGTST | | | |
| | | EPSEGSAPGTSESATPESGPGSEPAT | | | |
| | | SGSETPGTSTEPSEGSAPGTSTEPSE | | | |
| | | GSAPGTSESATPESGPGTSESATPES | | | |
| | | GPGSPAGSPTSTEEGTSESATPESGP | | | |
| | | GSEPATSGSETPGTSESATPESGPGT | | | |
| | | STEPSEGSAPGTSTEPSEGSAPGTST | | | |
| | | EPSEGSAPGTSTEPSEGSAPGTSTEP | | | |
| | | SEGSAPGTSTEPSEGSAPGSPAGSPT | | | |
| | | STEEGTSTEPSEGSAPGTSESATPES | | | |
| | | GPGSEPATSGSETPGTSESATPESGP | | | |
| | | GSEPATSGSETPGTSESATPESGPGT | | | |
| | | STEPSEGSAPGTSESATPESGPGSPA | | | |
| | | GSPTSTEEGSPAGSPTSTEEGSPAGS | | | |
| | | PTSTEEGTSESATPESGPGTSTEPSE | | | |
| | | GSAP | | | |
| AF540 | 303 | GSTSSTAESPGPGSTSSTAESPGPGS | 540 | Residue totals: H: 2 E: 0 | 99.65 |
| | | TSESPSGTAPGSTSSTAESPGPGSTS | | percent: H: 0.4 E: 0.0 | |
| | | STAESPGPGTSTPESGSASPGSTSES | | | |
| | | PSGTAPGTSPSGESSTAPGSTSESPS | | | |
| | | GTAPGSTSESPSGTAPGTSPSGESST | | | |
| | | APGSTSESPSGTAPGSTSESPSGTAP | | | |
| | | GTSPSGESSTAPGSTSESPSGTAPGS | | | |
| | | TSESPSGTAPGSTSESPSGTAPGTST | | | |
| | | PESGSASPGSTSESPSGTAPGTSTPE | | | |
| | | SGSASPGSTSSTAESPGPGSTSSTAE | | | |
| | | SPGPGTSTPESGSASPGTSTPESGSA | | | |
| | | SPGSTSESPSGTAPGTSTPESGSASP | | | |
| | | GTSTPESGSASPGSTSESPSGTAPGS | | | |
| | | TSESPSGTAPGSTSESPSGTAPGSTS | | | |
| | | STAESPGPGTSTPESGSASPGTSTPE | | | |
| | | SGSASPGSTSESPSGTAPGSTSESPS | | | |
| | | GTAPGTSTPESGSASPGSTSESPSGT | | | |
| | | APGSTSESPSGTAPGTSTPESGSASP | | | |
| | | GTSPSGESSTAPGSTSSTAESPGPGT | | | |
| | | SPSGESSTAPGSTSSTAESPGPGTST | | | |
| | | PESGSASPGSTSESPSGTAP | | | |
| AF504 | 304 | GASPGTSSTGSPGSSPSASTGTGPGS | 504 | Residue totals: H: 0 E: 0 | 94.44% |
| | | SPSASTGTGPGTPGSGTASSSPGSST | | percent: H: 0.0 E: 0.0 | |
| | | PSGATGSPGSNPSASTGTGPGASPGT | | | |
| | | SSTGSPGTPGSGTASSSPGSSTPSGA | | | |
| | | TGSPGTPGSGTASSSPGASPGTSSTG | | | |
| | | SPGASPGTSSTGSPGTPGSGTASSSP | | | |
| | | GSSTPSGATGSPGASPGTSSTGSPGT | | | |
| | | PGSGTASSSPGSSTPSGATGSPGSNP | | | |
| | | SASTGTGPGSSPSASTGTGPGSSTPS | | | |
| | | GATGSPGSSTPSGATGSPGASPGTSS | | | |
| | | TGSPGASPGTSSTGSPGASPGTSSTG | | | |
| | | SPGTPGSGTASSSPGASPGTSSTGSP | | | |
| | | GASPGTSSTGSPGASPGTSSTGSPGS | | | |
| | | SPSASTGTGPGTPGSGTASSSPGASP | | | |
| | | GTSSTGSPGASPGTSSTGSPGASPGT | | | |
| | | SSTGSPGSSTPSGATGSPGSSTPSGA | | | |
| | | TGSPGASPGTSSTGSPGTPGSGTASS | | | |
| | | SPGSSTPSGATGSPGSSTPSGATGSP | | | |
| | | GSSTPSGATGSPGSSPSASTGTGPGA | | | |
| | | SPGTSSTGSP | | | |
| AE864 | 305 | GSPAGSPTSTEEGTSESATPESGPGT | 864 | Residue totals: H: 2 E: 3 | 99.77% |
| | | STEPSEGSAPGSPAGSPTSTEEGTST | | percent: H: 0.2 E: 0.4 | |
| | | EPSEGSAPGTSTEPSEGSAPGTSESA | | | |
| | | TPESGPGSEPATSGSETPGSEPATSG | | | |
| | | SETPGSPAGSPTSTEEGTSESATPES | | | |
| | | GPGTSTEPSEGSAPGTSTEPSEGSAP | | | |
| | | GSPAGSPTSTEEGTSTEPSEGSAPGT | | | |
| | | STEPSEGSAPGTSESATPESGPGTST | | | |
| | | EPSEGSAPGTSESATPESGPGSEPAT | | | |
| | | SGSETPGTSTEPSEGSAPGTSTEPSE | | | |
| | | GSAPGTSESATPESGPGTSESATPES | | | |
| | | GPGSPAGSPISTEEGTSESATPESGP | | | |
| | | GSEPATSGSETPGTSESATPESGPGT | | | |
| | | STEPSEGSAPGTSTEPSEGSAPGTST | | | |
| | | EPSEGSAPGTSTEPSEGSAPGTSTEP | | | |
| | | SEGSAPGTSTEPSEGSAPGSPAGSPT | | | |
| | | STEEGTSTEPSEGSAPGTSESATPES | | | |
| | | GPGSEPATSGSETPGTSESAIPESGP | | | |
| | | GSEPATSGSETPGTSESATPESGPGT | | | |
| | | STEPSEGSAPGTSESATPESGPGSPA | | | |
| | | GSPTSTEEGSPAGSPTSTEEGSPAGS | | | |
| | | PTSTEEGTSESATPESGPGTSTEPSE | | | |
| | | GSAPGTSESATPESGPGSEPATSGSE | | | |
| | | TPGTSESATPESGPGSEPATSGSETP | | | |
| | | GTSESATPESGPGTSTEPSEGSAPGS | | | |
| | | PAGSPTSTEEGTSESATPESGPGSEP | | | |
| | | ATSGSETPGTSESATPESGPGSPAGS | | | |
| | | PTSTEEGSPAGSPTSTEEGTSTEPSE | | | |
| | | GSAPGTSESATPESGPGTSESATPES | | | |
| | | GPGTSESATPESGPGSEPATSGSETP | | | |
| | | GSEPATSGSETPGSPAGSPTSTEEGT | | | |
| | | STEPSEGSAPGTSTEPSEGSAPGSEP | | | |
| | | ATSGSETPGTSESATPESGPGTSTEP | | | |
| | | SEGSAP | | | |
| AF864 | 306 | GSTSESPSGTAPGTSPSGESSTAPGS | 875 | Residue totals: H: 2 E: 0 | 95.20% |
| | | TSESPSGTAPGSTSESPSGTAPGTST | | percent: H: 0.2 E: 0.0 | |
| | | PESGSASPGTSTPESGSASPGSTSES | | | |
| | | PSGTAPGSTSESPSGTAPGTSPSGES | | | |
| | | STAPGSTSESPSGTAPGTSPSGESST | | | |
| | | APGTSPSGESSTAPGSTSSTAESPGP | | | |
| | | GTSPSGESSTAPGTSPSGESSTAPGS | | | |
| | | TSSTAESPGPGTSTPESGSASPGTST | | | |
| | | PESGSASPGSTSESPSGTAPGSTSES | | | |
| | | PSGTAPGTSTPESGSASPGSTSSTAE | | | |
| | | SPGPGTSTPESGSASPGSTSESPSGT | | | |
| | | APGTSPSGESSTAPGSTSSTAESPGP | | | |
| | | GTSPSGESSTAPGTSTPESGSASPGS | | | |
| | | TSSTAESPGPGSTSSTAESPGPGSTS | | | |
| | | STAESPGPGSTSSTAESPGPGTSPSG | | | |
| | | ESSTAPGSTSESPSGTAPGSTSESPS | | | |
| | | GTAPGTSTPESGPXXXGASASGAPST | | | |
| | | XXXXSESPSGTAPGSTSESPSGTAPG | | | |
| | | STSESPSGTAPGSTSESPSGTAPGST | | | |
| | | SESPSGTAPGSTSESPSGTAPGTSTP | | | |
| | | ESGSASPGTSPSGESSTAPGTSPSGE | | | |
| | | SSTAPGSTSSTAESPGPGTSPSGESS | | | |
| | | TAPGTSTPESGSASPGSTSESPSGTA | | | |
| | | PGSTSESPSGTAPGTSPSGESSTAPG | | | |
| | | STSESPSGTAPGTSTPESGSASPGTS | | | |
| | | TRESGSASPGSTSESPSGTAPGTSTP | | | |
| | | ESGSASPGSTSSTAESPGPGSTSESP | | | |
| | | SGTAPGSTSESPSGTAPGTSPSGESS | | | |
| | | TAPGSTSSTAESPGPGTSPSGESSTA | | | |
| | | PGTSTPESGSASPGTSPSGESSTAPG | | | |
| | | TSPSGESSTAPGTSPSGESSTAPGST | | | |
| | | SSTAESPGPGSTSSTAESPGPGTSPS | | | |
| | | GESSTAPGSSPSASTGTGPGSSTPSG | | | |
| | | ATGSPGSSTPSGATGSP | | | |
| AG864 | 307 | GGSPGASPGTSSTGSPGSSPSASTGT | 868 | Residue totals: H: 0 E: 0 | 94.70% |
| | | GPGSSPSASTGTGPGTPGSGTASSSP | | percent: H: 0.0 E: 0.0 | |
| | | GSSTPSGATGSPGSNPSASTGTGPGA | | | |
| | | SPGTSSTGSPGTPGSGTASSSPGSST | | | |
| | | PSGATGSPGTPGSGTASSSPGASPGT | | | |
| | | SSTGSPGASPGTSSTGSPGTPGSGTA | | | |
| | | SSSPGSSTPSGATGSPGASPGTSSTG | | | |
| | | SPGTPGSGTASSSPGSSTPSGATGSP | | | |
| | | GSNPSASTGTGPGSSPSASTGTGPGS | | | |
| | | STPSGATGSPGSSTPSGATGSPGASP | | | |
| | | GTSSTGSPGASPGTSSTGSPGASPGT | | | |
| | | SSTGSPGTPGSGTASSSPGASPGTSS | | | |
| | | TGSPGASPGTSSTGSPGASPGTSSTG | | | |
| | | SPGSSPSASTGTGPGTPGSGTASSSP | | | |
| | | GASPGTSSTGSPGASPGTSSTGSPGA | | | |
| | | SPGTSSTGSPGSSTPSGATGSPGSST | | | |
| | | PSGATGSPGASPGTSSTGSPGTPGSG | | | |
| | | TASSSPGSSTPSGATGSPGSSTPSGA | | | |
| | | TGSPGSSTPSGATGSPGSSPSASTGT | | | |
| | | GPGASPGTSSTGSPGASPGTSSTGSP | | | |
| | | GTPGSGTASSSPGASPGTSSTGSPGA | | | |
| | | SPGTSSTGSPGASPGTSSTGSPGASP | | | |
| | | GTSSTGSPGTPGSGTASSSPGSSTPS | | | |
| | | GATGSPGTPGSGTASSSPGSSTPSGA | | | |
| | | TGSPGTPGSGTASSSPGSSTPSGATG | | | |
| | | SPGSSTPSGATGSPGSSPSASTGTGP | | | |
| | | GSSPSASTGTGPGASPGTSSTGSPGT | | | |
| | | PGSGTASSSPGSSTPSGATGSPGSSP | | | |
| | | SASTGTGPGSSPSASTGTGPGASPGT | | | |
| | | SSTGSPGASPGTSSTGSPGSSTPSGA | | | |
| | | TGSPGSSPSASTGTGPGASPGTSSTG | | | |
| | | SPGSSPSASTGTGPGTPGSGTASSSP | | | |
| | | GSSTPSGATGSPGSSTPSGATGSPGA | | | |
| | | SPGTSSTGSP | | | |
| AM875 | 308 | GTSTEPSEGSAPGSEPATSGSETPGS | 875 | Residue totals: H: 7 E: 3 | 98.63% |
| | | PAGSPTSTEEGSTSSTAESPGPGTST | | percent: H: 0.8 E: 0.3 | |
| | | PESGSASPGSTSESPSGTAPGSTSES | | | |
| | | PSGTAPGTSTPESGSASPGTSTPESG | | | |
| | | SASPGSEPATSGSETPGTSESATPES | | | |
| | | GPGSPAGSPTSTEEGTSTEPSEGSAP | | | |
| | | GTSESATPESGPGTSTEPSEGSAPGT | | | |
| | | STEPSEGSAPGSPAGSPTSTEEGTST | | | |
| | | EPSEGSAPGTSTEPSEGSAPGTSESA | | | |
| | | TPESGPGTSESATPESGPGTSTEPSE | | | |
| | | GSAPGTSTEPSEGSAPGTSESATPES | | | |
| | | GPGTSTEPSEGSAPGSEPATSGSETP | | | |
| | | GSPAGSPTSTEEGSSTPSGATGSPGT | | | |
| | | PGSGTASSSPGSSTPSGATGSPGTST | | | |
| | | EPSEGSAPGTSTEPSEGSAPGSEPAT | | | |
| | | SGSETPGSPAGSPTSTEEGSPAGSPT | | | |
| | | STEEGTSTEPSEGSAPGASASGAPST | | | |
| | | GGTSESATPESGPGSPAGSPTSTEEG | | | |
| | | SPAGSPTSTEEGSTSSTAESPGPGST | | | |
| | | SESPSGTAPGTSPSGESSTAPGTPGS | | | |
| | | GTASSSPGSSTPSGATGSPGSSPSAS | | | |
| | | TGTGPGSEPATSGSETPGTSESATPE | | | |
| | | SGPGSEPATSGSETPGSTSSTAESPG | | | |
| | | PGSTSSTAESPGPGTSPSGESSTAPG | | | |
| | | SEPATSGSETPGSEPATSGSETPGTS | | | |
| | | TEPSEGSAPGSTSSTAESPGPGTSTP | | | |
| | | ESGSASPGSTSESPSGTAPGTSTEPS | | | |
| | | EGSAPGTSTEPSEGSAPGTSTEPSEG | | | |
| | | SAPGSSTPSGATGSPGSSPSASTGTG | | | |
| | | PGASPGTSSTGSPGSEPATSGSETPG | | | |
| | | TSESATPESGPGSPAGSPTSTEEGSS | | | |
| | | TPSGATGSPGSSPSASTGTGPGASPG | | | |
| | | TSSTGSPGTSESATPESGPGTSTEPS | | | |
| | | EGSAPGTSTEPSEGSAP | | | |
| AM1296 | 309 | GTSTEPSEGSAPGSEPATSGSETPGS | 1318 | Residue totals: H: 7 E: 0 | 99.17% |
| | | PAGSPTSTEEGSTSSTAESPGPGTST | | percent: H: 0.7 E: 0.0 | |
| | | PESGSASPGSTSESPSGTAPGSTSES | | | |
| | | PSGTAPGTSTPESGSASPGTSTPESG | | | |
| | | SASPGSEPATSGSETPGTSESATPES | | | |
| | | GPGSPAGSPTSTEEGTSTEPSEGSAP | | | |
| | | GTSESATPESGPGTSTEPSEGSAPGT | | | |
| | | STEPSEGSAPGSPAGSPTSTEEGTST | | | |
| | | EPSEGSAPGTSTEPSEGSAPGTSESA | | | |
| | | TPESGPGTSESATPESGPGTSTEPSE | | | |
| | | GSAPGTSTEPSEGSAPGTSESATPES | | | |
| | | GPGTSTEPSEGSAPGSEPATSGSETP | | | |
| | | GSPAGSPTSTEEGSSTPSGATGSPGT | | | |
| | | PGSGTASSSPGSSTPSGATGSPGTST | | | |
| | | EPSEGSAPGTSTEPSEGSAPGSEPAT | | | |
| | | SGSETPGSPAGSPTSTEEGSPAGSPT | | | |
| | | STEEGTSTEPSEGSAPGPEPTGPAPS | | | |
| | | GGSEPATSGSETPGTSESATPESGPG | | | |
| | | SPAGSPTSTEEGTSESATPESGPGSP | | | |
| | | AGSPTSTEEGSPAGSPTSTEEGTSES | | | |
| | | ATPESGPGSPAGSPTSTEEGSPAGSP | | | |
| | | TSTEEGSTSSTAESPGPGSTSESPSG | | | |
| | | TAPGTSPSGESSTAPGSTSESPSGTA | | | |
| | | PGSTSESPSGTAPGTSPSGESSTAPG | | | |
| | | TSTEPSEGSAPGTSESATPESGPGTS | | | |
| | | ESATPESGPGSEPATSGSETPGTSES | | | |
| | | ATPESGPGTSESATPESGPGTSTEPS | | | |
| | | EGSAPGTSESATPESGPGTSTEPSEG | | | |
| | | SAPGTSPSGESSTAPGTSPSGESSTA | | | |
| | | PGTSPSGESSTAPGTSTEPSEGSAPG | | | |
| | | SPAGSPTSTEEGTSTEPSEGSAPGSS | | | |
| | | PSASTGTGPGSSTPSGATGSPGSSTP | | | |
| | | SGATGSPGSSTPSGATGSPGSSTPSG | | | |
| | | ATGSPGASPGTSSTGSPGASASGAPS | | | |
| | | TGGTSPSGESSTAPGSTSSTAESPGP | | | |
| | | GTSPSGESSTAPGTSESATPESGPGT | | | |
| | | STEPSEGSAPGTSTEPSEGSAPGSSP | | | |
| | | SASTGTGPGSSTPSGATGSPGASPGT | | | |
| | | SSTGSPGTSTPESGSASPGTSPSGES | | | |
| | | STAPGTSPSGESSTAPGTSESATPES | | | |
| | | GPGSEPATSGSETPGTSTEPSEGSAP | | | |
| | | GSTSESPSGTAPGSTSESPSGTAPGT | | | |
| | | STPESGSASPGSPAGSPTSTEEGTSE | | | |
| | | SATPESGPGTSTEPSEGSAPGSPAGS | | | |
| | | PTSTEEGTSESATPESGPGSEPATSG | | | |
| | | SETPGSSTPSGATGSPGASPGTSSTG | | | |
| | | SPGSSTPSGATGSPGSTSESPSGTAP | | | |
| | | GTSPSGESSTAPGSTSSTAESPGPGS | | | |
| | | STPSGATGSPGASPGTSSTGSPGTPG | | | |
| | | SGTASSSPGSPAGSPTSTEEGSPAGS | | | |
| | | PTSTEEGTSTEPSEGSAP | | | |
| AM923 | 310 | MAEPAGSPTSTEEGASPGTSSTGSPG | 924 | Residue totals: H: 4 E: 3 | 98.70% |
| | | SSTPSGATGSPGSSTPSGAIGSPGTS | | percent: H: 0.4 E: 0.3 | |
| | | TEPSEGSAPGSEPATSGSETPGSPAG | | | |
| | | SPTSTEEGSTSSTAESPGPGTSTPES | | | |
| | | GSASPGSTSESPSGTAPGSTSESPSG | | | |
| | | TAPGTSTPESGSASPGTSTPESGSAS | | | |
| | | PGSEPATSGSETPGTSESATPESGPG | | | |
| | | SPAGSPTSTEEGTSTEPSEGSAPGTS | | | |
| | | ESATPESGPGTSTEPSEGSAPGTSTE | | | |
| | | PSEGSAPGSPAGSPTSTEEGTSTEPS | | | |
| | | EGSAPGTSTEPSEGSAPGTSESATPE | | | |
| | | SGPGTSESATPESGPGTSTEPSEGSA | | | |
| | | PGTSTEPSEGSAPGTSESATPESGPG | | | |
| | | TSTEPSEGSAPGSEPATSGSETPGSP | | | |
| | | AGSPTSTEEGSSTPSGATGSPGTPGS | | | |
| | | GTASSSPGSSTPSGATGSPGTSTEPS | | | |
| | | EGSAPGTSTEPSEGSAPGSEPATSGS | | | |
| | | ETPGSPAGSPTSTEEGSPAGSPTSTE | | | |
| | | EGTSTEPSEGSAPGASASGAPSTGGT | | | |
| | | SESATPESGPGSPAGSPTSTEEGSPA | | | |
| | | GSPTSTEEGSTSSTAESPGPGSTSES | | | |
| | | PSGTAPGTSPSGESSTAPGTPGSGTA | | | |
| | | SSSPGSSTPSGATGSPGSSPSASTGT | | | |
| | | GPGSEPATSGSETPGTSESATPESGP | | | |
| | | GSEPATSGSETPGSTSSTAESPGPGS | | | |
| | | TSSTAESPGPGTSPSGESSTAPGSEP | | | |
| | | ATSGSETPGSEPATSGSETPGTSTEP | | | |
| | | SEGSAPGSTSSTAESPGPGTSTPESG | | | |
| | | SASPGSTSESPSGTAPGTSTEPSEGS | | | |
| | | APGTSTEPSEGSAPGTSTEPSEGSAP | | | |
| | | GSSTPSGATGSPGSSPSASTGTGPGA | | | |
| | | SPGTSSTGSPGSEPATSGSETPGTSE | | | |
| | | SATPESGPGSPAGSPTSTEEGSSTPS | | | |
| | | GATGSPGSSPSASTGTGPGASPGTSS | | | |
| | | TGSPGTSESATPESGPGTSTEPSEGS | | | |
| | | APGTSTEPSEGSAP | | | |
| AE912 | 311 | MAEPAGSPTSTEEGTPGSGTASSSPG | 913 | Residue totals: H: 8 E: 3 | 99.45% |
| | | SSTPSGATGSPGASPGTSSTGSPGSP | | percent: H: 0.9 E: 0.3 | |
| | | AGSPTSTEEGTSESATPESGPGTSTE | | | |
| | | PSEGSAPGSPAGSPTSTEEGTSTEPS | | | |
| | | EGSAPGTSTEPSEGSAPGTSESATPE | | | |
| | | SGPGSEPATSGSETPGSEPATSGSET | | | |
| | | PGSPAGSPTSTEEGTSESATPESGPG | | | |
| | | TSTEPSEGSAPGTSTEPSEGSAPGSP | | | |
| | | AGSPTSTEEGTSTEPSEGSAPGTSTE | | | |
| | | PSEGSAPGTSESATPESGPGTSTEPS | | | |
| | | EGSAPGTSESATPESGPGSEPATSGS | | | |
| | | ETPGTSTEPSEGSAPGTSTEPSEGSA | | | |
| | | PGTSESATPESGPGTSESATPESGPG | | | |
| | | SPAGSPTSTEEGTSESATPESGPGSE | | | |
| | | PATSGSETPGTSESATPESGPGTSTE | | | |
| | | PSEGSAPGTSTEPSEGSAPGTSTEPS | | | |
| | | EGSAPGTSTEPSEGSAPGTSTEPSEG | | | |
| | | SAPGTSTEPSEGSAPGSPAGSPTSTE | | | |
| | | EGTSTEPSEGSAPGTSESATPESGPG | | | |
| | | SEPATSGSETPGTSESATPESGPGSE | | | |
| | | PATSGSETPGTSESATPESGPGTSTE | | | |
| | | PSEGSAPGTSESATPESGPGSPAGSP | | | |
| | | TSTEEGSPAGSPTSTEEGSPAGSPTS | | | |
| | | TEEGTSESATPESGPGTSTEPSEGSA | | | |
| | | PGTSESATPESGPGSEPATSGSETPG | | | |
| | | TSESATPESGPGSEPATSGSETPGTS | | | |
| | | ESATPESGPGTSTEPSEGSAPGSPAG | | | |
| | | SPTSTEEGTSESATPESGPGSEPATS | | | |
| | | GSETPGTSESATPESGPGSPAGSPTS | | | |
| | | TEEGSPAGSPTSTEEGTSTEPSEGSA | | | |
| | | PGTSESATPESGPGTSESATPESGPG | | | |
| | | TSESATPESGPGSEPATSGSETPGSE | | | |
| | | PATSGSETPGSPAGSPTSTEEGTSTE | | | |
| | | PSEGSAPGTSTEPSEGSAPGSEPATS | | | |
| | | GSETPGTSESATPESGPGTSTEPSEG | | | |
| | | SAP | | | |
| BC 864 | 312 | GTSTEPSEPGSAGTSTEPSEPGSAGS | | Residue totals: H: 0 E: 0 | 99.77% |
| | | EPATSGTEPSGSGASEPTSTEPGSEP | | percent: H: 0 E: 0 | |
| | | ATSGTEPSGSEPATSGTEPSGSEPAT | | | |
| | | SGTEPSGSGASEPTSTEPGTSTEPSE | | | |
| | | PGSAGSEPATSGTEPSGTSTEPSEPG | | | |
| | | SAGSEPATSGTEPSGSEPATSGTEPS | | | |
| | | GTSTEPSEPGSAGTSTEPSEPGSAGS | | | |
| | | EPATSGTEPSGSEPATSGTEPSGTSE | | | |
| | | PSTSEPGAGSGASEPTSTEPGTSEPS | | | |
| | | TSEPGAGSEPATSGTEPSGSEPATSG | | | |
| | | TEPSGTSTEPSEPGSAGTSTEPSEPG | | | |
| | | SAGSGASEPTSTEPGSEPATSGTEPS | | | |
| | | GSEPATSGTEPSGSEPATSGTEPSGS | | | |
| | | EPATSGTEPSGTSTEPSEPGSAGSEP | | | |
| | | ATSGTEPSGSGASEPTSTEPGTSTEP | | | |
| | | SEPGSAGSEPATSGTEPSGSGASEPT | | | |
| | | STEPGTSTEPSEPGSAGSGASEPTST | | | |
| | | EPGSEPATSGTEPSGSGASEPTSTEP | | | |
| | | GSEPATSGTEPSGSGASEPTSTEPGT | | | |
| | | STEPSEPGSAGSEPATSGTEPSGSGA | | | |
| | | SEPTSTEPGTSTEPSEPGSAGSEPAT | | | |
| | | SGTEPSGTSTEPSEPGSAGSEPATSG | | | |
| | | TEPSGTSTEPSEPGSAGTSTEPSEPG | | | |
| | | SAGTSTEPSEPGSAGTSTEPSEPGSA | | | |
| | | GTSTEPSEPGSAGTSTEPSEPGSAGT | | | |
| | | SEPSTSEPGAGSGASEPTSTEPGTST | | | |
| | | EPSEPGSAGTSTEPSEPGSAGTSTEP | | | |
| | | SEPGSAGSEPATSGTEPSGSGASEPT | | | |
| | | STEPGSEPATSGTEPSGSEPATSGTE | | | |
| | | PSGSEPATSGTEPSGSEPATSGTEPS | | | |
| | | GTSEPSTSEPGAGSEPATSGTEPSGS | | | |
| | | GASEPTSTEPGTSTEPSEPGSAGSEP | | | |
| | | ATSGTEPSGSGASEPTSTEPGTSTEP | | | |
| | | SEPGSA | | | |
| | 313 | ASPAAPAPASPAAPAPSAPAAAPASP | 84 | Residue totals: H: 58 E: 0 | 78.57% |
| | | APAAPSAPAPAAPSAASPAAPSAPPA | | percent: H: 69.0 E: 0.0 | |
| | | AASPAAPSAPPAASAAAPAAASAAAS | | | |
| | | APSAAA | | | |

*H: alpha-helix E: beta-sheet

### Example 6: Analysis of Polypeptide Sequences for Repetitiveness

Polypeptide amino acid sequences can be assessed for repetitiveness by quantifying the number of times a shorter subsequence appears within the overall polypeptide. For example, a polypeptide of 200 amino acid residues has 192 overlapping 9-amino acid subsequences (or 9-mer “frames”), but the number of unique 9-mer subsequences will depend on the amount of repetitiveness within the sequence. In the present analysis, different sequences were assessed for repetitiveness by summing the occurrence of all unique 3-mer subsequences for each 3-amino acid frame across the first 200 amino acids of the polymer portion divided by the absolute number of unique 3-mer subsequences within the 200 amino acid sequence. The resulting subsequence score is a reflection of the degree of repetitiveness within the polypeptide.

The results, shown in Table 11, indicate that the unstructured polypeptides consisting of 2 or 3 amino acid types have high subsequence scores, while those of consisting of 12 amino acids motifs of the six amino acids G, S, T, E, P, and A with a low degree of internal repetitiveness, have subsequence scores of less than 10, and in some cases, less than 5. For example, the L288 sequence has two amino acid types and has short, highly repetitive sequences, resulting in a subsequence score of 50.0. The polypeptide J288 has three amino acid types but also has short, repetitive sequences, resulting in a subsequence score of 33.3. Y576 also has three amino acid types, but is not made of internal repeats, reflected in the subsequence score of 15.7 over the first 200 amino acids. W576 consists of four types of amino acids, but has a higher degree of internal repetitiveness, e.g., “GGSG” (SEQ ID NO: 270), resulting in a subsequence score of 23.4. The AD576 consists of four types of 12 amino acid motifs, each consisting of four types of amino acids. Because of the low degree of internal repetitiveness of the individual motifs, the overall subsequence score over the first 200 amino acids is 13.6. In contrast, XTEN's consisting of four motifs contains six types of amino acids, each with a low degree of internal repetitiveness have lower subsequence scores; e.g., AE864 (6.1), AF864 (7.5), and AM875 (4.5).

Conclusions: The results indicate that the combination of 12 amino acid subsequence motifs, each consisting of four to six amino acid types that are essentially non-repetitive, into a longer XTEN polypeptide results in an overall sequence that is non-repetitive. This is despite the fact that each subsequence motif may be used multiple times across the sequence. In contrast, polymers created from smaller numbers of amino acid types resulted in higher subsequence scores, although the actual sequence can be tailored to reduce the degree of repetitiveness to result in lower subsequence scores.

TABLE 11
Subsequence score calculations of polypeptide sequences

| Seq | SEQ ID | | |
|---|---|---|---|
| Name | NO: | Amino Acid Sequence | Score |
| J288 | 314 | GSGGEGGSGGEGGSGGEGGSGGEGGSGGEGGSGGEGGSGGEGGSGGEGGSGGEGG | 33.3 |
| | | SGGEGGSGGEGGSGGEGGSGGEGGSGGEGGSGGEGGSGGEGGSGGEGGSGGEGGS | |
| | | GGEGGSGGEGGSGGEGGSGGEGGSGGEGGSGGEGGSGGEGGSGGEGGSGGEGGSG | |
| | | GEGGSGGEGGSGGEGGSGGEGGSGGEGGSGGEGGSGGEGGSGGEGGSGGEGGSGG | |
| | | EGGSGGEGGSGGEGGSGGEGGSGGEGGSGGEGGSGGEGGSGGEGGSGGEGGSGGE | |
| | | GGSGGEGGSGGEG | |
| K288 | 315 | GEGEGGGEGGEGEGGGEGGEGEGGGEGGEGEGGGEGGEGEGGGEGGEGEGGGEGG | 46.9 |
| | | EGEGGGEGGEGEGGGEGGEGEGGGEGGEGEGGGEGGEGEGGGEGGEGEGGGEGGE | |
| | | GEGGGEGGEGEGGGEGGEGEGGGEGGEGEGGGEGGEGEGGGEGGEGEGGGEGGEG | |
| | | EGGGEGGEGEGGGEGGEGEGGGEGGEGEGGGEGGEGEGGGEGGEGEGGGEGGEGE | |
| | | GGGEGGEGEGGGEGGEGEGGGEGGEGEGGGEGGEGEGGGEGGEGEGGGEGGEGEG | |
| | | GGEGGEGEGGGEG | |
| L288 | 316 | SSESSESSSSESSSESSESSSSESSSESSESSSSESSSESSESSSSESSSESSES | 50.0 |
| | | SSSESSSESSESSSSESSSESSESSSSESSSESSESSSSESSSESSESSSSESSS | |
| | | ESSESSSSESSSESSESSSSESSSESSESSSSESSSESSESSSSESSSESSESSS | |
| | | SESSSESSESSSSESSSESSESSSSESSSESSESSSSESSSESSESSSSESSSES | |
| | | SESSSSESSSESSESSSSESSSESSESSSSESSSESSESSSSESSSESSESSSSE | |
| | | SSSESSESSSSES | |
| Y288 | 317 | GEGSGEGSEGEGSEGSGEGEGSEGSGEGEGGSEGSEGEGGSEGSEGEGGSEGSEG | 26.8 |
| | | EGSGEGSEGEGGSEGSEGEGSGEGSEGEGSEGGSEGEGGSEGSEGEGSGEGSEGE | |
| | | GGEGGSEGEGSEGSGEGEGSGEGSEGEGSEGSGEGEGSGEGSEGEGSEGSGEGEG | |
| | | SEGSGEGEGGSEGSEGEGSEGSGEGEGGEGSGEGEGSGEGSEGEGGGEGSEGEGS | |
| | | GEGGEGEGSEGGSEGEGGSEGGEGEGSEGSGEGEGSEGGSEGEGSEGGSEGEGSE | |
| | | GSGEGEGSEGSGE | |
| Q576 | 318 | GGKPGEGGKPEGGGGKPGGKPEGEGEGKPGGKPEGGGKPGGGEGGKPEGGKPEGE | 18.5 |
| | | GKPGGGEGKPGGKPEGGGGKPEGEGKPGGGGGKPGGKPEGEGKPGGGEGGKPEGK | |
| | | PGEGGEGKPGGKPEGGGEGKPGGGKPGEGGKPGEGKPGGGEGGKPEGGKPEGEGK | |
| | | PGGGEGKPGGKPGEGGKPEGGGEGKPGGKPGEGGEGKPGGGKPEGEGKPGGGKPG | |
| | | GGEGGKPEGEGKPGGKPEGGGEGKPGGKPEGGGKPEGGGEGKPGGGKPGEGGKPG | |
| | | EGEGKPGGKPEGEGKPGGEGGGKPEGKPGGGEGGKPEGGKPGEGGKPEGGKPGEG | |
| | | GEGKPGGGKPGEGGKPEGGGKPEGEGKPGGGGKPGEGGKPEGGKPEGGGEGKPGG | |
| | | GKPEGEGKPGGGEGKPGGKPEGGGGKPGEGGKPEGGKPGGEGGGKPEGEGKPGGK | |
| | | PGEGGGGKPGGKPEGEGKPGEGGEGKPGGKPEGGGEGKPGGKPEGGGEGKPGGGK | |
| | | PGEGGKPEGGGKPGEGGKPGEGGKPEGEGKPGGGEGKPGGKPGEGGKPEGGGEGK | |
| | | PGGKPGGEGGGKPEGGKPGEGGKPEG | |
| U576 | 319 | GEGKPGGKPGSGGGKPGEGGKPGSGEGKPGGKPGSGGSGKPGGKPGEGGKPEGGS | 18.1 |
| | | GGKPGGGGKPGGKPGGEGSGKPGGKPEGGGKPEGGSGGKPGGKPEGGSGGKPGGK | |
| | | PGSGEGGKPGGGKPGGEGKPGSGKPGGEGSGKPGGKPEGGSGGKPGGKPEGGSGG | |
| | | KPGGSGKPGGKPGEGGKPEGGSGGKPGGSGKPGGKPEGGGSGKPGGKPGEGGKPG | |
| | | SGEGGKPGGGKPGGEGKPGSGKPGGEGSGKPGGKPGSGGEGKPGGKPEGGSGGKP | |
| | | GGGKPGGEGKPGSGGKPGEGGKPGSGGGKPGGKPGGEGEGKPGGKPGEGGKPGGE | |
| | | GSGKPGGGGKPGGKPGGEGGKPEGSGKPGGGSGKPGGKPEGGGGKPEGSGKPGGG | |
| | | GKPEGSGKPGGGKPEGGSGGKPGGSGKPGGKPGEGGGKPEGSGKPGGGSGKPGGK | |
| | | PEGGGKPEGGSGGKPGGKPEGGSGGKPGGKPGGEGSGKPGGKPGSGEGGKPGGKP | |
| | | GEGSGGKPGGKPEGGSGGKPGGSGKPGGKPEGGGSGKPGGKPGEGGKPGGEGSGK | |
| | | PGGSGKPG | |
| W576 | 320 | GGSGKPGKPGGSGSGKPGSGKPGGGSGKPGSGKPGGGSGKPGSGKPGGGSGKPGS | 23.4 |
| | | GKPGGGGKPGSGSGKPGGGKPGGSGGKPGGGSGKPGKPGSGGSGKPGSGKPGGGS | |
| | | GGKPGKPGSGGSGGKPGKPGSGGGSGKPGKPGSGGSGGKPGKPGSGGSGGKPGKP | |
| | | GSGGSGKPGSGKPGGGSGKPGSGKPGSGGSGKPGKPGSGGSGKPGSGKPGSGSGK | |
| | | PGSGKPGGGSGKPGSGKPGSGGSGKPGKPGSGGGKPGSGSGKPGGGKPGSGSGKP | |
| | | GGGKPGGSGGKPGGSGGKPGKPGSGGGSGKPGKPGSGGGSGKPGKPGGSGSGKPG | |
| | | SGKPGGGSGKPGSGKPGSGGSGKPGKPGSGGSGGKPGKPGSGGGKPGSGSGKPGG | |
| | | GKPGSGSGKPGGGKPGSGSGKPGGGKPGSGSGKPGGSGKPGSGKPGGGSGGKPGK | |
| | | PGSGGSGKPGSGKPGSGGSGKPGKPGGSGSGKPGSGKPGGGSGKPGSGKPGGGSG | |
| | | KPGSGKPGGGSGKPGSGKPGGGGKPGSGSGKPGGSGGKPGKPGSGGSGGKPGKPG | |
| | | SGGSGKPGSGKPGGGSGGKPGKPGSGG | |
| Y576 | 321 | GEGSGEGSEGEGSEGSGEGEGSEGSGEGEGGSEGSEGEGSEGSGEGEGGEGSGEG | 15.7 |
| | | EGSGEGSEGEGGGEGSEGEGSGEGGEGEGSEGGSEGEGGSEGGEGEGSEGSGEGE | |
| | | GSEGGSEGEGSEGGSEGEGSEGSGEGEGSEGSGEGEGSEGSGEGEGSEGSGEGEG | |
| | | SEGGSEGEGGSEGSEGEGSGEGSEGEGGSEGSEGEGGGEGSEGEGSGEGSEGEGG | |
| | | SEGSEGEGGSEGSEGEGGEGSGEGEGSEGSGEGEGSGEGSEGEGSEGSGEGEGSE | |
| | | GSGEGEGGSEGSEGEGSGEGSEGEGSEGSGEGEGSEGSGEGEGGSEGSEGEGGSE | |
| | | GSEGEGGSEGSEGEGGEGSGEGEGSEGSGEGEGSGEGSEGEGSEGSGEGEGSEGS | |
| | | GEGEGGSEGSEGEGSEGSGEGEGGEGSGEGEGSGEGSEGEGGGEGSEGEGSEGSG | |
| | | EGEGSEGSGEGEGSEGGSEGEGGSEGSEGEGSEGGSEGEGSEGGSEGEGSEGSGE | |
| | | GEGSEGSGEGEGSGEGSEGEGGSEGGEGEGSEGGSEGEGSEGGSEGEGGEGSGEG | |
| | | EGGGEGSEGEGSEGSGEGEGSGEGSE | |
| AD576 | 322 | GSSESGSSEGGPGSGGEPSESGSSGSSESGSSEGGPGSSESGSSEGGPGSSESGS | 13.6 |
| | | SEGGPGSSESGSSEGGPGSSESGSSEGGPGESPGGSSGSESGSEGSSGPGESSGS | |
| | | SESGSSEGGPGSSESGSSEGGPGSSESGSSEGGPGSGGEPSESGSSGESPGGSSG | |
| | | SESGESPGGSSGSESGSGGEPSESGSSGSSESGSSEGGPGSGGEPSESGSSGSGG | |
| | | EPSESGSSGSEGSSGPGESSGESPGGSSGSESGSGGEPSESGSSGSGGEPSESGS | |
| | | SGSGGEPSESGSSGSSESGSSEGGPGESPGGSSGSESGESPGGSSGSESGESPGG | |
| | | SSGSESGESPGGSSGSESGESPGGSSGSESGSSESGSSEGGPGSGGEPSESGSSG | |
| | | SEGSSGPGESSGSSESGSSEGGPGSGGEPSESGSSGSSESGSSEGGPGSGGEPSE | |
| | | SGSSGESPGGSSGSESGESPGGSSGSESGSSESGSSEGGPGSGGEPSESGSSGSS | |
| | | ESGSSEGGPGSGGEPSESGSSGSGGEPSESGSSGESPGGSSGSESGSEGSSGPGE | |
| | | SSGSSESGSSEGGPGSEGSSGPGESS | |
| AE576 | 323 | AGSPAGSPTSTEEGTSESATPESGPGTSTEPSEGSAPGSPAGSPTSTEEGTSTEP | 6.1 |
| | | SEGSAPGTSTEPSEGSAPGTSESATPESGPGSEPATSGSETPGSEPATSGSETPG | |
| | | SPAGSPTSTEEGTSESATPESGPGTSTEPSEGSAPGTSTEPSEGSAPGSPAGSPT | |
| | | STEEGTSTEPSEGSAPGTSTEPSEGSAPGTSESATPESGPGTSTEPSEGSAPGTS | |
| | | ESATPESGPGSEPATSGSETPGTSTEPSEGSAPGTSTEPSEGSAPGTSESATPES | |
| | | GPGTSESATPESGPGSPAGSPTSTEEGTSESATPESGPGSEPATSGSETPGTSES | |
| | | ATPESGPGTSTEPSEGSAPGTSTEPSEGSAPGTSTEPSEGSAPGTSTEPSEGSAP | |
| | | GTSTEPSEGSAPGTSTEPSEGSAPGSPAGSPTSTEEGTSTEPSEGSAPGTSESAT | |
| | | PESGPGSEPATSGSETPGTSESATPESGPGSEPATSGSETPGTSESATPESGPGT | |
| | | STEPSEGSAPGTSESATPESGPGSPAGSPTSTEEGSPAGSPTSTEEGSPAGSPTS | |
| | | TEEGTSESATPESGPGTSTEPSEGSAP | |
| AF540 | 324 | GSTSSTAESPGPGSTSSTAESPGPGSTSESPSGTAPGSTSSTAESPGPGSTSSTA | 8.8 |
| | | ESPGPGTSTPESGSASPGSTSESPSGTAPGTSPSGESSTAPGSTSESPSGTAPGS | |
| | | TSESPSGTAPGTSPSGESSTAPGSTSESPSGTAPGSTSESPSGTAPGTSPSGESS | |
| | | TAPGSTSESPSGTAPGSTSESPSGTAPGSTSESPSGTAPGTSTPESGSASPGSTS | |
| | | ESPSGTAPGTSTPESGSASPGSTSSTAESPGPGSTSSTAESPGPGTSTPESGSAS | |
| | | PGTSTPESGSASPGSTSESPSGTAPGTSTPESGSASPGTSTPESGSASPGSTSES | |
| | | PSGTAPGSTSESPSGTAPGSTSESPSGTAPGSTSSTAESPGPGTSTPESGSASPG | |
| | | TSTPESGSASPGSTSESPSGTAPGSTSESPSGTAPGTSTPESGSASPGSTSESPS | |
| | | GTAPGSTSESPSGTAPGTSTPESGSASPGTSPSGESSTAPGSTSSTAESPGPGTS | |
| | | PSGESSTAPGSTSSTAESPGPGTSTPESGSASPGSTSESPSGTAP | |
| AF504 | 325 | GASPGTSSTGSPGSSPSASTGTGPGSSPSASTGTGPGTPGSGTASSSPGSSTPSG | 7.0 |
| | | ATGSPGSNPSASTGTGPGASPGTSSTGSPGTPGSGTASSSPGSSTPSGATGSPGT | |
| | | PGSGTASSSPGASPGTSSTGSPGASPGTSSTGSPGTPGSGTASSSPGSSTPSGAT | |
| | | GSPGASPGTSSTGSPGTPGSGTASSSPGSSTPSGATGSPGSNPSASTGTGPGSSP | |
| | | SASTGTGPGSSTPSGATGSPGSSTPSGATGSPGASPGTSSTGSPGASPGTSSTGS | |
| | | PGASPGTSSTGSPGTPGSGTASSSPGASPGTSSTGSPGASPGTSSTGSPGASPGT | |
| | | SSTGSPGSSPSASTGTGPGTPGSGTASSSPGASPGTSSTGSPGASPGTSSTGSPG | |
| | | ASPGTSSTGSPGSSTPSGATGSPGSSTPSGATGSPGASPGTSSTGSPGTPGSGTA | |
| | | SSSPGSSTPSGATGSPGSSTPSGATGSPGSSTPSGATGSPGSSPSASTGTGPGAS | |
| | | PGTSSTGSP | |
| AE864 | 326 | GSPAGSPTSTEEGTSESATPESGPGTSTEPSEGSAPGSPAGSPTSTEEGTSTEPS | 6.1 |
| | | EGSAPGTSTEPSEGSAPGTSESATPESGPGSEPATSGSETPGSEPATSGSETPGS | |
| | | PAGSPTSTEEGTSESATPESGPGTSTEPSEGSAPGTSTEPSEGSAPGSPAGSPTS | |
| | | TEEGTSTEPSEGSAPGTSTEPSEGSAPGTSESATPESGPGTSTEPSEGSAPGTSE | |
| | | SATPESGPGSEPATSGSETPGTSTEPSEGSAPGTSTEPSEGSAPGTSESATPESG | |
| | | PGTSESATPESGPGSPAGSPTSTEEGTSESATPESGPGSEPATSGSETPGTSESA | |
| | | TPESGPGTSTEPSEGSAPGTSTEPSEGSAPGTSTEPSEGSAPGTSTEPSEGSAPG | |
| | | TSTEPSEGSAPGTSTEPSEGSAPGSPAGSPTSTEEGTSTEPSEGSAPGTSESATP | |
| | | ESGPGSEPATSGSETPGTSESATPESGPGSEPATSGSETPGTSESATPESGPGTS | |
| | | TEPSEGSAPGTSESATPESGPGSPAGSPTSTEEGSPAGSPTSTEEGSPAGSPTST | |
| | | EEGTSESATPESGPGTSTEPSEGSAPGTSESATPESGPGSEPATSGSETPGTSES | |
| | | ATPESGPGSEPATSGSETPGTSESATPESGPGTSTEPSEGSAPGSPAGSPTSTEE | |
| | | GTSESATPESGPGSEPATSGSETPGTSESATPESGPGSPAGSPTSTEEGSPAGSP | |
| | | TSTEEGTSTEPSEGSAPGTSESATPESGPGTSESATPESGPGTSESATPESGPGS | |
| | | EPATSGSETPGSEPATSGSETPGSPAGSPTSTEEGTSTEPSEGSAP | |
| AF864 | 327 | GSTSESPSGTAPGTSPSGESSTAPGSTSESPSGTAPGSTSESPSGTAPGTSTPES | 7.5 |
| | | GSASPGTSTPESGSASPGSTSESPSGTAPGSTSESPSGTAPGTSPSGESSTAPGS | |
| | | TSESPSGTAPGTSPSGESSTAPGTSPSGESSTAPGSTSSTAESPGPGTSPSGESS | |
| | | TAPGTSPSGESSTAPGSTSSTAESPGPGTSTPESGSASPGTSTPESGSASPGSTS | |
| | | ESPSGTAPGSTSESPSGTAPGTSTPESGSASPGSTSSTAESPGPGTSTPESGSAS | |
| | | PGSTSESPSGTAPGTSPSGESSTAPGSTSSTAESPGPGTSPSGESSTAPGTSTPE | |
| | | SGSASPGSTSSTAESPGPGSTSSTAESPGPGSTSSTAESPGPGSTSSTAESPGPG | |
| | | TSPSGESSTAPGSTSESPSGTAPGSTSESPSGTAPGTSTPESGPXXXGASASGAP | |
| | | STXXXXSESPSGTAPGSTSESPSGTAPGSTSESPSGTAPGSTSESPSGTAPGSTS | |
| | | ESPSGTAPGSTSESPSGTAPGTSTPESGSASPGTSPSGESSTAPGTSPSGESSTA | |
| | | PGSTSSTAESPGPGTSPSGESSTAPGTSTPESGSASPGSTSESPSGTAPGSTSES | |
| | | PSGTAPGTSPSGESSTAPGSTSESPSGTAPGTSTPESGSASPGTSTPESGSASPG | |
| | | STSESPSGTAPGTSTPESGSASPGSTSSTAESPGPGSTSESPSGTAPGSTSESPS | |
| | | GTAPGTSPSGESSTAPGSTSSTAESPGPGTSPSGESSTAPGTSTPESGSASPGTS | |
| | | PSGESSTAPGTSPSGESSTAPGTSPSGESSTAPGSTSSTAESPGPGSTSSTAESP | |
| | | GPGTSPSGESSTAPGSSPSASTGTGPGSSTPSGATGSPGSSTPSGATGSP | |
| AG868 | 328 | GGSPGASPGTSSTGSPGSSPSASTGTGPGSSPSASTGTGPGTPGSGTASSSPGSS | 7.5 |
| | | TPSGATGSPGSNPSASTGTGPGASPGTSSTGSPGTPGSGTASSSPGSSTPSGATG | |
| | | SPGTPGSGTASSSPGASPGTSSTGSPGASPGTSSTGSPGTPGSGTASSSPGSSTP | |
| | | SGATGSPGASPGTSSTGSPGTPGSGTASSSPGSSTPSGATGSPGSNPSASTGTGP | |
| | | GSSPSASTGTGPGSSTPSGATGSPGSSTPSGATGSPGASPGTSSTGSPGASPGTS | |
| | | STGSPGASPGTSSTGSPGTPGSGTASSSPGASPGTSSTGSPGASPGTSSTGSPGA | |
| | | SPGTSSTGSPGSSPSASTGTGPGTPGSGTASSSPGASPGTSSTGSPGASPGTSST | |
| | | GSPGASPGTSSTGSPGSSTPSGATGSPGSSTPSGATGSPGASPGTSSTGSPGTPG | |
| | | SGTASSSPGSSTPSGATGSPGSSTPSGATGSPGSSTPSGATGSPGSSPSASTGTG | |
| | | PGASPGTSSTGSPGASPGTSSTGSPGTPGSGTASSSPGASPGTSSTGSPGASPGT | |
| | | SSTGSPGASPGTSSTGSPGASPGTSSTGSPGTPGSGTASSSPGSSTPSGATGSPG | |
| | | TPGSGTASSSPGSSTPSGATGSPGTPGSGTASSSPGSSTPSGATGSPGSSTPSGA | |
| | | TGSPGSSPSASTGTGPGSSPSASTGTGPGASPGTSSTGSPGTPGSGTASSSPGSS | |
| | | TPSGATGSPGSSPSASTGTGPGSSPSASTGTGPGASPGTSSTGSPGASPGTSSTG | |
| | | SPGSSTPSGATGSPGSSPSASTGTGPGASPGTSSTGSPGSSPSASTGTGPGTPGS | |
| | | GTASSSPGSSTPSGATGSPGSSTPSGATGSPGASPGTSSTGSP | |
| AM875 | 329 | GTSTEPSEGSAPGSEPATSGSETPGSPAGSPTSTEEGSTSSTAESPGPGTSTPES | 4.5 |
| | | GSASPGSTSESPSGTAPGSTSESPSGTAPGTSTPESGSASPGTSTPESGSASPGS | |
| | | EPATSGSETPGTSESATPESGPGSPAGSPTSTEEGTSTEPSEGSAPGTSESATPE | |
| | | SGPGTSTEPSEGSAPGTSTEPSEGSAPGSPAGSPTSTEEGTSTEPSEGSAPGTST | |
| | | EPSEGSAPGTSESATPESGPGTSESATPESGPGTSTEPSEGSAPGTSTEPSEGSA | |
| | | PGTSESATPESGPGTSTEPSEGSAPGSEPATSGSETPGSPAGSPTSTEEGSSTPS | |
| | | GATGSPGTPGSGTASSSPGSSTPSGATGSPGTSTEPSEGSAPGTSTEPSEGSAPG | |
| | | SEPATSGSETPGSPAGSPTSTEEGSPAGSPTSTEEGTSTEPSEGSAPGASASGAP | |
| | | STGGTSESATPESGPGSPAGSPTSTEEGSPAGSPTSTEEGSTSSTAESPGPGSTS | |
| | | ESPSGTAPGTSPSGESSTAPGTPGSGTASSSPGSSTPSGATGSPGSSPSASTGTG | |
| | | PGSEPATSGSETPGTSESATPESGPGSEPATSGSETPGSTSSTAESPGPGSTSST | |
| | | AESPGPGTSPSGESSTAPGSEPATSGSETPGSEPATSGSETPGTSTEPSEGSAPG | |
| | | STSSTAESPGPGTSTPESGSASPGSTSESPSGTAPGTSTEPSEGSAPGTSTEPSE | |
| | | GSAPGTSTEPSEGSAPGSSTPSGATGSPGSSPSASTGTGPGASPGTSSTGSPGSE | |
| | | PATSGSETPGTSESATPESGPGSPAGSPTSTEEGSSTPSGATGSPGSSPSASTGT | |
| | | GPGASPGTSSTGSPGTSESATPESGPGTSTEPSEGSAPGTSTEPSEGSAP | |
| AM1296 | 330 | GTSTEPSEGSAPGSEPATSGSETPGSPAGSPTSTEEGSTSSTAESPGPGTSTPES | 4.5 |
| | | GSASPGSTSESPSGTAPGSTSESPSGTAPGTSTPESGSASPGTSTPESGSASPGS | |
| | | EPATSGSETPGTSESATPESGPGSPAGSPTSTEEGTSTEPSEGSAPGTSESATPE | |
| | | SGPGTSTEPSEGSAPGTSTEPSEGSAPGSPAGSPTSTEEGTSTEPSEGSAPGTST | |
| | | EPSEGSAPGTSESATPESGPGTSESATPESGPGTSTEPSEGSAPGTSTEPSEGSA | |
| | | PGTSESATPESGPGTSTEPSEGSAPGSEPATSGSETPGSPAGSPTSTEEGSSTPS | |
| | | GATGSPGTPGSGTASSSPGSSTPSGATGSPGTSTEPSEGSAPGTSTEPSEGSAPG | |
| | | SEPATSGSETPGSPAGSPTSTEEGSPAGSPTSTEEGTSTEPSEGSAPGPEPTGPA | |
| | | PSGGSEPATSGSETPGTSESATPESGPGSPAGSPTSTEEGTSESATPESGPGSPA | |
| | | GSPTSTEEGSPAGSPTSTEEGTSESATPESGPGSPAGSPTSTEEGSPAGSPTSTE | |
| | | EGSTSSTAESPGPGSTSESPSGTAPGTSPSGESSTAPGSTSESPSGTAPGSTSES | |
| | | PSGTAPGTSPSGESSTAPGTSTEPSEGSAPGTSESATPESGPGTSESATPESGPG | |
| | | SEPATSGSETPGTSESATPESGPGTSESATPESGPGTSTEPSEGSAPGTSESATP | |
| | | ESGPGTSTEPSEGSAPGTSPSGESSTAPGTSPSGESSTAPGTSPSGESSTAPGTS | |
| | | TEPSEGSAPGSPAGSPTSTEEGTSTEPSEGSAPGSSPSASTGTGPGSSTPSGATG | |
| | | SPGSSTPSGATGSPGSSTPSGATGSPGSSTPSGATGSPGASPGTSSTGSPGASAS | |
| | | GAPSTGGTSPSGESSTAPGSTSSTAESPGPGTSPSGESSTAPGTSESATPESGPG | |
| | | TSTEPSEGSAPGTSTEPSEGSAPGSSPSASTGTGPGSSTPSGATGSPGASPGTSS | |
| | | TGSPGTSTPESGSASPGTSPSGESSTAPGTSPSGESSTAPGTSESATPESGPGSE | |
| | | PATSGSETPGTSTEPSEGSAPGSTSESPSGTAPGSTSESPSGTAPGTSTPESGSA | |
| | | SPGSPAGSPTSTEEGTSESATPESGPGTSTEPSEGSAPGSPAGSPTSTEEGTSES | |
| | | ATPESGPGSEPATSGSETPGSSTPSGATGSPGASPGTSSTGSPGSSTPSGATGSP | |
| | | GSTSESPSGTAPGTSPSGESSTAPGSTSSTAESPGPGSSTPSGATGSPGASPGTS | |
| | | STGSPGTPGSGTASSSPGSPAGSPTSTEEGSPAGSPTSTEEGTSTEPSEGSAP | |

### Example 7: Calculation of TEPITOPE Scores

TEPITOPE scores of 9mer peptide sequence can be calculated by adding pocket potentials as described by Sturniolo [Sturniolo, T., et al. (1999) Nat Biotechnol, 17: 555]. In the present Example, separate Tepitope scores were calculated for individual HLA alleles. To calculate the TEPITOPE score of a peptide with sequence P1-P2-P3-P4-P5-P6-P7-P8-P9, the corresponding individual pocket potentials in Table 12 were added. The HLA*0101B score of a 9mer peptide with the sequence FDKLPRTSG (SEQ ID NO: 271) would be the sum of 0, −1.3, 0, 0.9, 0, −1.8, 0.09, 0, 0.

To evaluate the TEPITOPE scores for long peptides one can repeat the process for all 9mer subsequences of the sequences. This process can be repeated for the proteins encoded by other HLA alleles. Tables 13-16 give pocket potentials for the protein products of HLA alleles that occur with high frequency in the Caucasian population.

TEPITOPE scores calculated by this method range from approximately −10 to +10. However, 9mer peptides that lack a hydrophobic amino acid (FKLMVWVY (SEQ ID NO: 272)) in P1 position have calculated TEPITOPE scores in the range of −1009 to −989. This value is biologically meaningless and reflects the fact that a hydrophobic amino acid serves as an anchor residue for HLA binding and peptides lacking a hydrophobic residue in P1 are considered non binders to HLA. Because most XTEN sequences lack hydrophobic residues, all combinations of 9mer subsequences will have TEPITOPEs in the range in the range of −1009 to −989. This method confirms that XTEN polypeptides may have few or no predicted T-cell epitopes.

TABLE 12
Pocket potential for HLA*0101B allele.

| Amino | | | | | | | | | |
|---|---|---|---|---|---|---|---|---|---|
| Acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −2.4 | — | −2.7 | −2 | — | −1.9 |
| E | −999 | 0.1 | −1.2 | −0.4 | — | −2.4 | −0.6 | — | −1.9 |
| F | 0 | 0.8 | 0.8 | 0.08 | — | −2.1 | 0.3 | — | −0.4 |
| G | −999 | 0.5 | 0.2 | −0.7 | — | −0.3 | −1.1 | — | −0.8 |
| H | −999 | 0.8 | 0.2 | −0.7 | — | −2.2 | 0.1 | — | −1.1 |
| I | −1 | 1.1 | 1.5 | 0.5 | — | −1.9 | 0.6 | — | 0.7 |
| K | −999 | 1.1 | 0 | −2.1 | — | 2 | −0.2 | — | −1.7 |
| L | −1 | 1 | 1 | 0.9 | — | −2 | 0.3 | — | 0.5 |
| M | −1 | 1.1 | 1.4 | 0.8 | — | −1.8 | 0.09 | — | 0.08 |
| N | −999 | 0.8 | 0.5 | 0.04 | — | −1.1 | 0.1 | — | −1.2 |
| P | −999 | −0.5 | 0.3 | −1.9 | — | −0.2 | 0.07 | — | −1.1 |
| Q | −999 | 1.2 | 0 | 0.1 | — | −1.8 | 0.2 | — | 1.6 |
| R | −999 | 2.2 | 0.7 | −2.1 | — | −1.8 | 0.09 | — | −1 |
| S | −999 | −0.3 | 0.2 | −0.7 | — | −0.6 | −0.2 | — | −0.3 |
| T | −999 | 0 | 0 | −1 | — | −1.2 | 0.09 | — | −0.2 |
| V | −1 | 2.1 | 0.5 | −0.1 | — | −1.1 | 0.7 | — | 0.3 |
| W | 0 | −0.1 | 0 | −1.8 | — | −2.4 | −0.1 | — | −1.4 |
| Y | 0 | 0.9 | 0.8 | −1.1 | — | −2 | 0.5 | — | −0.9 |

TABLE 13
Pocket potential for HLA*0301B allele.

| Amino | | | | | | | | | |
|---|---|---|---|---|---|---|---|---|---|
| Acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | 2.3 | — | −2.4 | −0.6 | — | −0.6 |
| E | −999 | 0.1 | −1.2 | −1 | — | −1.4 | −0.2 | — | −0.3 |
| F | −1 | 0.8 | 0.8 | −1 | — | −1.4 | 0.5 | — | 0.9 |
| G | −999 | 0.5 | 0.2 | 0.5 | — | −0.7 | 0.1 | — | 0.4 |
| H | −999 | 0.8 | 0.2 | 0 | — | −0.1 | −0.8 | — | −0.5 |
| I | 0 | 1.1 | 1.5 | 0.5 | — | 0.7 | 0.4 | — | 0.6 |
| K | −999 | 1.1 | 0 | −1 | — | 1.3 | −0.9 | — | −0.2 |
| L | 0 | 1 | 1 | 0 | — | 0.2 | 0.2 | — | −0 |
| M | 0 | 1.1 | 1.4 | 0 | — | −0.9 | 1.1 | — | 1.1 |
| N | −999 | 0.8 | 0.5 | 0.2 | — | −0.6 | −0.1 | — | −0.6 |
| P | −999 | −0.5 | 0.3 | −1 | — | 0.5 | 0.7 | — | −0.3 |
| Q | −999 | 1.2 | 0 | 0 | — | −0.3 | −0.1 | — | −0.2 |
| R | −999 | 2.2 | 0.7 | −1 | — | 1 | −0.9 | — | 0.5 |
| S | −999 | −0.3 | 0.2 | 0.7 | — | −0.1 | 0.07 | — | 1.1 |
| T | −999 | 0 | 0 | −1 | — | 0.8 | −0.1 | — | −0.5 |
| V | 0 | 2.1 | 0.5 | 0 | — | 1.2 | 0.2 | — | 0.3 |
| W | −1 | −0.1 | 0 | −1 | — | −1.4 | −0.6 | — | −1 |
| Y | −1 | 0.9 | 0.8 | −1 | — | −1.4 | −0.1 | — | 0.3 |

TABLE 14
Pocket potential for HLA*0401B allele.

| Amino | | | | | | | | | |
|---|---|---|---|---|---|---|---|---|---|
| Acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | 1.4 | — | −1.1 | −0.3 | — | −1.7 |
| E | −999 | 0.1 | −1.2 | 1.5 | — | −2.4 | 0.2 | — | −1.7 |
| F | 0 | 0.8 | 0.8 | −0.9 | — | −1.1 | −1 | — | −1 |
| G | −999 | 0.5 | 0.2 | −1.6 | — | −1.5 | −1.3 | — | −1 |
| H | −999 | 0.8 | 0.2 | 1.1 | — | −1.4 | 0 | — | 0.08 |
| I | −1 | 1.1 | 1.5 | 0.8 | — | −0.1 | 0.08 | — | −0.3 |
| K | −999 | 1.1 | 0 | −1.7 | — | −2.4 | −0.3 | — | −0.3 |
| L | −1 | 1 | 1 | 0.8 | — | −1.1 | 0.7 | — | −1 |
| M | −1 | 1.1 | 1.4 | 0.9 | — | −1.1 | 0.8 | — | −0.4 |
| N | −999 | 0.8 | 0.5 | 0.9 | — | 1.3 | 0.6 | — | −1.4 |
| P | −999 | −0.5 | 0.3 | −1.6 | — | 0 | −0.7 | — | −1.3 |
| Q | −999 | 1.2 | 0 | 0.8 | — | −1.5 | 0 | — | 0.5 |
| R | −999 | 2.2 | 0.7 | −1.9 | — | −2.4 | −1.2 | — | −1 |
| S | −999 | −0.3 | 0.2 | 0.8 | — | 1 | −0.2 | — | 0.7 |
| T | −999 | 0 | 0 | 0.7 | — | 1.9 | −0.1 | — | −1.2 |
| V | 1 | 2.1 | 0.5 | −0.9 | — | 0.9 | 0.08 | — | −0.7 |
| W | 0 | −0.1 | 0 | −1.2 | — | −1 | −1.4 | — | −1 |
| Y | 0 | 0.9 | 0.8 | −1.6 | — | −1.5 | −1.2 | — | −1 |

TABLE 15
Pocket potential for HLA*0701B allele.

| Amino | | | | | | | | | |
|---|---|---|---|---|---|---|---|---|---|
| Acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −1.6 1 | — | −2.5 | −1.3 | — | −1.2 |
| E | −999 | 0.1 | −1.2 | −1.4 | — | −2.5 | 0.9 | — | −0.3 |
| F | 0 | 0.8 | 0.8 | 0.2 | — | −0.8 | 2.1 1 | — | 2.1 |
| G | −999 | 0.5 | 0.2 | −1.1 | — | −0.6 | 0 | — | −0.6 |
| H | −999 | 0.8 | 0.2 | 0.1 | — | −0.8 | 0.9 | — | −0.2 |
| I | −1 | 1.1 | 1.5 | 1.1 | — | −0.5 | 2.4 | — | 3.4 |
| K | −999 | 1.1 | 0 | −1.3 | — | −1.1 | 0.5 | — | −1.1 |
| L | −1 | 1 | 1 | −0.8 | — | −0.9 | 2.2 | — | 3.4 |
| M | −1 | 1.1 | 1.4 | −0.4 | — | −0.8 | 1.8 | — | 2 |
| N | −999 | 0.8 | 0.5 | −1.1 | — | −0.6 | 1.4 | — | −0.5 |
| P | −999 | −0.5 | 0.3 | −1.2 | — | −0.5 | −0.2 | — | −0.6 |
| Q | −999 | 1.2 | 0 | −1.5 | — | −1.1 | 1.1 | — | −0.9 |
| R | −999 | 2.2 | 0.7 | −1.1 | — | −1.1 | 0.7 | — | −0.8 |
| S | −999 | −0.3 | 0.2 | 1.5 | — | 0.6 | 0.4 | — | −0.3 |
| T | −999 | 0 | 0 | 1.4 | — | −0.1 | 0.9 | — | 0.4 |
| V | −1 | 2.1 | 0.5 | 0.9 | — | 0.1 | 1.6 | — | 2 |
| W | 0 | −0.1 | 0 | −1.1 | — | −0.9 | 1.4 | — | 0.8 |
| Y | 0 | 0.9 | 0.8 | −0.9 | — | −1 | 1.7 | — | 1.1 |

TABLE 16
Pocket potential for HLA*1501B allele.

| Amino | | | | | | | | | |
|---|---|---|---|---|---|---|---|---|---|
| Acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −0.4 | — | −0.4 | −0.7 | — | −1.9 |
| E | −999 | 0.1 | −1.2 | −0.6 | — | −1 | −0.7 | — | −1.9 |
| F | −1 | 0.8 | 0.8 | 2.4 | — | −0.3 | 1.4 | — | −0.4 |
| G | −999 | 0.5 | 0.2 | 0 | — | 0.5 | 0 | — | −0.8 |
| H | −999 | 0.8 | 0.2 | 1.1 | — | −0.5 | 0.6 | — | −1.1 |
| I | 0 | 1.1 | 1.5 | 0.6 | — | 0.05 | 1.5 | — | 0.7 |
| K | −999 | 1.1 | 0 | −0.7 | — | −0.3 | −0.3 | — | −1.7 |
| L | 0 | 1 | 1 | 0.5 | — | 0.2 | 1.9 | — | 0.5 |
| M | 0 | 1.1 | 1.4 | 1 | — | 0.1 | 1.7 | — | 0.08 |
| N | −999 | 0.8 | 0.5 | 0.2 | — | 0.7 | 0.7 | — | −1.2 |
| P | −999 | −0.5 | 0.3 | −0.3 | — | −0.2 | 0.3 | — | −1.1 |
| Q | −999 | 1.2 | 0 | −0.8 | — | −0.8 | −0.3 | — | −1.6 |
| R | −999 | 2.2 | 0.7 | 0.2 | — | 1 | −0.5 | — | −1 |
| S | −999 | −0.3 | 0.2 | −0.3 | — | 0.6 | 0.3 | — | −0.3 |
| T | −999 | 0 | 0 | −0.3 | — | 0 | 0.2 | — | −0.2 |
| V | 0 | 2.1 | 0.5 | 0.2 | — | −0.3 | 0.3 | — | 0.3 |
| W | −1 | −0.1 | 0 | 0.4 | — | −0.4 | 0.6 | — | −1.4 |
| Y | −1 | 0.9 | 0.8 | 2.5 | — | 0.4 | 0.7 | — | −0.9 |

TABLE 17
Exemplary Biological Activity, Exemplary Assays and Preferred Indications for BP

| Biologically | | | |
|---|---|---|---|
| Active Protein | Biological Activity | Exemplary Activity Assay | Preferred Indication: |
| IL-1 receptor | Binds IL1 receptor | Competition for IL-1 binding to | Autoimmune Disease; Arthritis; |
| antagonist | without activating the | IL-1 receptors in YT-NCI or | Rheumatoid Arthritis; Asthma; |
| (Anakinra; soluble | target cells; inhibits the | C3H/HeJ cells (Carter et al., | Diabetes; Diabetes Mellitus; |
| interleukin-1 | binding of IL1-alpha | Nature 344: 633-638, 1990); | GVHD; Inflammatory Bowel |
| receptor; IRAP; | and IL1-beta; and | Inhibition of IL-1-induced | Disorders; Chron's Disease; |
| KINERET; | neutralizes the biologic | endothelial cell-leukocyte | Ocular Inflammation; Psoriasis; |
| ANTRIL) | activity of IL1-alpha | adhesion (Carter et al., Nature | Septic Shock; Transplant |
| | and IL1-beta. | 344: 633-638, 1990); | Rejection; Inflammatory |
| | | Proliferation assays on A375- | Disorders; Rheumatic Disorders; |
| | | C6 cells, a human melanoma | Osteoporosis; Postmenopausal |
| | | cell line highly susceptible to | Osteoporosis; Stroke. |
| | | the antiproliferative action of | |
| | | IL-1 (Murai T et al., J. Biol. | |
| | | Chem. 276: 6797-6806, 2001). | |
| IL-10 receptor | Binds IL10 receptor; | Conformational Changes | Autoimmune Disease; Arthritis; |
| agonist | facilitates the | Mediate Interleukin−10 | Rheumatoid Arthritis; Asthma; |
| | interaction between IL- | Receptor 2(IL-10R2) Binding | Diabetes; Diabetes Mellitus; |
| | 10R1 and IL-10R2, | to IL-10 and Assembly of the | GVHD; Inflammatory Bowel |
| | leading to downstream | Signaling Complex (Yoon S. et | Disorders; Chron's Disease; |
| | signalling that results in | al., J. Biol. Chem. 281: 35088- | Ocular Inflammation; Psoriasis; |
| | anti-inflammatory | 35096, 2006). | Septic Shock; Transplant |
| | response. | | Rejection; Inflammatory |
| | | | Disorders; Rheumatic Disorder; |
| | | | Osteoporosis; Postmenopausal |
| | | | Osteoporosis |

TABLE 18
Exemplary BPXTEN of linked to XTEN

| BPXTEN | SEQ ID | |
|---|---|---|
| Name* | NO: | Sequence |
| IL-1ra- | 331 | MRPSGRKSSKMQAFRIWDVNQKTFYLRNNQLVAGYLQGPNVNLEEKIDVVPIEPHALFLGI |
| AE864 | | HGGKMCLSCVKSGDETRLQLEAVNITDLSENRKQDKRFAFIRSDSGPTTSFESAACPGWFL |
| | | CTAMEADQPVSLTNMPDEGVMVTKFYFQEDEGGSPAGSPTSTEEGTSESATPESGPGTSTE |
| | | PSEGSAPGSPAGSPTSTEEGTSTEPSEGSAPGTSTEPSEGSAPGTSESATPESGPGSEPAT |
| | | SGSETPGSEPATSGSETPGSPAGSPTSTEEGTSESATPESGPGTSTEPSEGSAPGTSTEPS |
| | | EGSAPGSPAGSPTSTEEGTSTEPSEGSAPGTSTEPSEGSAPGTSESATPESGPGTSTEPSE |
| | | GSAPGTSESATPESGPGSEPATSGSETPGTSTEPSEGSAPGTSTEPSEGSAPGTSESATPE |
| | | SGPGTSESATPESGPGSPAGSPTSTEEGTSESATPESGPGSEPATSGSETPGTSESATPES |
| | | GPGTSTEPSEGSAPGTSTEPSEGSAPGTSTEPSEGSAPGTSTEPSEGSAPGTSTEPSEGSA |
| | | PGTSTEPSEGSAPGSPAGSPTSTEEGTSTEPSEGSAPGTSESATPESGPGSEPATSGSETP |
| | | GTSESATPESGPGSEPATSGSETPGTSESATPESGPGTSTEPSEGSAPGTSESATPESGPG |
| | | SPAGSPTSTEEGSPAGSPTSTEEGSPAGSPTSTEEGTSESATPESGPGTSTEPSEGSAPGT |
| | | SESATPESGPGSEPATSGSETPGTSESATPESGPGSEPATSGSETPGTSESATPESGPGTS |
| | | TEPSEGSAPGSPAGSPTSTEEGTSESATPESGPGSEPATSGSETPGTSESATPESGPGSPA |
| | | GSPTSTEEGSPAGSPTSTEEGTSTEPSEGSAPGTSESATPESGPGTSESATPESGPGTSES |
| | | ATPESGPGSEPATSGSETPGSEPATSGSETPGSPAGSPTSTEEGTSTEPSEGSAPGTSTEP |
| | | SEGSAPGSEPATSGSETPGTSESATPESGPGTSTEPSEGSAP |
| IL-1ra- | 332 | MRPSGRKSSKMQAFRIWDVNQKTFYLRNNQLVAGYLQGPNVNLEEKIDVVPIEPHALFLGI |
| AM875 | | HGGKMCLSCVKSGDETRLQLEAVNITDLSENRKQDKRFAFIRSDSGPTTSFESAACPGWFL |
| | | CTAMEADQPVSLTNMPDEGVMVTKFYFQEDEGGTSTEPSEGSAPGSEPATSGSETPGSPAG |
| | | SPTSTEEGSTSSTAESPGPGTSTPESGSASPGSTSESPSGTAPGSTSESPSGTAPGTSTPE |
| | | SGSASPGTSTPESGSASPGSEPATSGSETPGTSESATPESGPGSPAGSPTSTEEGTSTEPS |
| | | EGSAPGTSESATPESGPGTSTEPSEGSAPGTSTEPSEGSAPGSPAGSPTSTEEGTSTEPSE |
| | | GSAPGTSTEPSEGSAPGTSESATPESGPGTSESATPESGPGTSTEPSEGSAPGTSTEPSEG |
| | | SAPGTSESATPESGPGTSTEPSEGSAPGSEPATSGSETPGSPAGSPTSTEEGSSTPSGATG |
| | | SPGTPGSGTASSSPGSSTPSGATGSPGTSTEPSEGSAPGTSTEPSEGSAPGSEPATSGSET |
| | | PGSPAGSPTSTEEGSPAGSPTSTEEGTSTEPSEGSAPGASASGAPSTGGTSESATPESGPG |
| | | SPAGSPTSTEEGSPAGSPTSTEEGSTSSTAESPGPGSTSESPSGTAPGTSPSGESSTAPGT |
| | | PGSGTASSSPGSSTPSGATGSPGSSPSASTGTGPGSEPATSGSETPGTSESATPESGPGSE |
| | | PATSGSETPGSTSSTAESPGPGSTSSTAESPGPGTSPSGESSTAPGSEPATSGSETPGSEP |
| | | ATSGSETPGTSTEPSEGSAPGSTSSTAESPGPGTSTPESGSASPGSTSESPSGTAPGTSTE |
| | | PSEGSAPGTSTEPSEGSAPGTSTEPSEGSAPGSSTPSGATGSPGSSPSASTGTGPGASPGT |
| | | SSTGSPGSEPATSGSETPGTSESATPESGPGSPAGSPTSTEEGSSTPSGATGSPGSSPSAS |
| | | TGTGPGASPGTSSTGSPGTSESATPESGPGTSTEPSEGSAPGTSTEPSEGSAP |
| IL-10- | 333 | MHSSALLCCLVLLTGVRASPGQGTQSENSCTHFPGNLPNMLRDLRDAFSRVKTFFQMKDQL |
| AE864 | | DNLLLKESLLEDFKGYLGCQALSEMIQFYLEEVMPQAENQDPDIKAHVNSLGENLKTLRLR |
| | | LRRCHRFLPCENKSKAVEQVKNAFNKLQEKGIYKAMSEFDIFINYTEAYMTMKIRNGSPAG |
| | | SPTSTEEGTSESATPESGPGTSTEPSEGSAPGSPAGSPTSTEEGTSTEPSEGSAPGTSTEP |
| | | SEGSAPGTSESATPESGPGSEPATSGSETPGSEPATSGSETPGSPAGSPTSTEEGTSESAT |
| | | PESGPGTSTEPSEGSAPGTSTEPSEGSAPGSPAGSPTSTEEGTSTEPSEGSAPGTSTEPSE |
| | | GSAPGTSESATPESGPGTSTEPSEGSAPGTSESATPESGPGSEPATSGSETPGTSTEPSEG |
| | | SAPGTSTEPSEGSAPGTSESATPESGPGTSESATPESGPGSPAGSPTSTEEGTSESATPES |
| | | GPGSEPATSGSETPGTSESATPESGPGTSTEPSEGSAPGTSTEPSEGSAPGTSTEPSEGSA |
| | | PGTSTEPSEGSAPGTSTEPSEGSAPGTSTEPSEGSAPGSPAGSPTSTEEGTSTEPSEGSAP |
| | | GTSESATPESGPGSEPATSGSETPGTSESATPESGPGSEPATSGSETPGTSESATPESGPG |
| | | TSTEPSEGSAPGTSESATPESGPGSPAGSPTSTEEGSPAGSPTSTEEGSPAGSPTSTEEGT |
| | | SESATPESGPGTSTEPSEGSAPGTSESATPESGPGSEPATSGSETPGTSESATPESGPGSE |
| | | PATSGSETPGTSESATPESGPGTSTEPSEGSAPGSPAGSPTSTEEGTSESATPESGPGSEP |
| | | ATSGSETPGTSESATPESGPGSPAGSPTSTEEGSPAGSPTSTEEGTSTEPSEGSAPGTSES |
| | | ATPESGPGTSESATPESGPGTSESATPESGPGSEPATSGSETPGSEPATSGSETPGSPAGS |
| | | PTSTEEGTSTEPSEGSAPGTSTEPSEGSAPGSEPATSGSETPGTSESATPESGPGTSTEPS |
| | | EGSAP |
| AM875- | 334 | GTSTEPSEGSAPGSEPATSGSETPGSPAGSPTSTEEGSTSSTAESPGPGTSTPESGSASPG |
| IL-10 | | STSESPSGTAPGSTSESPSGTAPGTSTPESGSASPGTSTPESGSASPGSEPATSGSETPGT |
| | | SESATPESGPGSPAGSPTSTEEGTSTEPSEGSAPGTSESATPESGPGTSTEPSEGSAPGTS |
| | | TEPSEGSAPGSPAGSPTSTEEGTSTEPSEGSAPGTSTEPSEGSAPGTSESATPESGPGTSE |
| | | SATPESGPGTSTEPSEGSAPGTSTEPSEGSAPGTSESATPESGPGTSTEPSEGSAPGSEPA |
| | | TSGSETPGSPAGSPTSTEEGSSTPSGATGSPGTPGSGTASSSPGSSTPSGATGSPGTSTEP |
| | | SEGSAPGTSTEPSEGSAPGSEPATSGSETPGSPAGSPTSTEEGSPAGSPTSTEEGTSTEPS |
| | | EGSAPGASASGAPSTGGTSESATPESGPGSPAGSPTSTEEGSPAGSPTSTEEGSTSSTAES |
| | | PGPGSTSESPSGTAPGTSPSGESSTAPGTPGSGTASSSPGSSTPSGATGSPGSSPSASTGT |
| | | GPGSEPATSGSETPGTSESATPESGPGSEPATSGSETPGSTSSTAESPGPGSTSSTAESPG |
| | | PGTSPSGESSTAPGSEPATSGSETPGSEPATSGSETPGTSTEPSEGSAPGSTSSTAESPGP |
| | | GTSTPESGSASPGSTSESPSGTAPGTSTEPSEGSAPGTSTEPSEGSAPGTSTEPSEGSAPG |
| | | SSTPSGATGSPGSSPSASTGTGPGASPGTSSTGSPGSEPATSGSETPGTSESATPESGPGS |
| | | PAGSPTSTEEGSSTPSGATGSPGSSPSASTGTGPGASPGTSSTGSPGTSESATPESGPGTS |
| | | TEPSEGSAPGTSTEPSEGSAPMHSSALLCCLVLLTGVRASPGQGTQSENSCTHFPGNLPNM |
| | | LRDLRDAFSRVKTFFQMKDQLDNLLLKESLLEDFKGYLGCQALSEMIQFYLEEVMPQAENQ |
| | | DPDIKAHVNSLGENLKTLRLRLRRCHRFLPCENKSKAVEQVKNAFNKLQEKGIYKAMSEFD |
| | | IFINYIEAYMTMKIRN |

*Sequence name reflects N- to C-terminus configuration of BP and XTEN components

TABLE B
DNA and amino acid sequences of an exemplified XTENylated IL-12 construct and a
reference construct.

| | | |
|---|---|---|
| Exemplified | DNA | GCATCACATCATCACCATCACCATCACCATGGTTCTCCAGCCGGGTCCCCAACTTC |
| XTENylated | sequence | GACCGAGGAAGGGACCTCCGAGTCAGCTACCCCGGAGTCCGGTCCTGGCACCTCCA |
| IL-12 | SEQ ID | CCGAACCATCGGAGGGCAGCGCCCCTGGGAGCCCTGCCGGGAGCCCTACAAGCACC |
| construct | NO: 1 | GAAGAGGGCACCAGTACAGAGCCAAGTGAGGGGAGCGCCCCTGGTACTAGTACTGA |
| (N-terminal | | ACCATCCGAGGGGTCAGCTCCAGGCACGAGTGAGTCCGCTACCCCCGAGAGCGGAC |
| His-tag is | | CGGGCTCAGAGCCCGCCACGAGTGGCAGTGAAACTCCAGGCTCAGAACCCGCCACT |
| optional) | | AGTGGGTCAGAGACTCCAGGCAGCCCTGCCGGATCCCCTACGTCCACCGAGGAGGG |
| | | AACATCTGAGTCCGCAACACCCGAATCCGGTCCAGGCACCTCCACGGAACCTAGTG |
| | | AAGGCTCGGCACCAGGTACAAGCACCGAACCTAGCGAGGGCAGCGCTCCCGGCAGC |
| | | CCTGCCGGCAGCCCAACCTCAACTGAGGAGGGCACCAGTACTGAGCCCAGCGAGGG |
| | | ATCAGCACCTGGCACCAGCACCGAACCTAGCGAGGGGAGCGCCCCTGGGACTAGCG |
| | | AGTCAGCTACACCAGAGAGCGGGCCTGGAACTTCTACCGAACCCAGTGAGGGATCC |
| | | GCTCCAGGCACCTCCGAATCCGCAACCCCCGAATCCGGACCTGGCTCAGAGCCCGC |
| | | CACCAGCGGGAGCGAAACCCCTGGCACATCCACCGAGCCTAGCGAAGGGTCCGCAC |
| | | CCGGCACCAGTACAGAGCCTAGCGAGGGATCAGCACCTGGCACCAGTGAATCTGCT |
| | | ACACCAGAGAGCGGCCCTGGAACCTCCGAGTCCGCTACCCCCGAGAGCGGGCCAGG |
| | | TTCTCCTGCTGGCTCCCCCACCTCAACAGAAGAGGGGACAAGCGAAAGCGCTACGC |
| | | CTGAGAGTGGCCCTGGCTCTGAGCCAGCCACCTCCGGCTCTGAAACCCCTGGCACT |
| | | AGTGAGTCTGCCACGCCTGAGTCCGGACCCGGGACCTCTACTGAGCCCTCGGAGGG |
| | | GAGCGCTCCTGGCACGAGTACAGAACCTTCCGAAGGAAGTGCACCGGGCACAAGCA |
| | | CCGAGCCTTCCGAAGGCTCTGCTCCCGGAACCTCTACCGAACCCTCTGAAGGGTCT |
| | | GCACCCGGCACGAGCACCGAACCCAGCGAAGGGTCAGCGCCTGGGACCTCAACAGA |
| | | GCCCTCGGAAGGATCAGCGCCTGGAAGCCCTGCAGGGAGTCCAACTTCCACGGAAG |
| | | AAGGAACGTCTACAGAGCCATCAGAGGGGTCCGCACCAGGTACCAGCGAATCCGCT |
| | | ACTCCCGAATCTGGCCCTGGGTCCGAACCTGCCACCTCCGGCTCTGAAACTCCAGG |
| | | GACCTCCGAATCTGCCACACCCGAGAGCGGCCCTGGCTCCGAGCCCGCAACATCTG |
| | | GCAGCGAGACACCTGGCACCTCCGAGAGCGCAACACCCGAGAGCGGCCCTGGCACC |
| | | AGCACCGAGCCATCCGAGGGATCCGCCCCAGGCACTTCTGAGTCAGCCACACCCGA |
| | | AAGCGGACCAGGATCACCCGCTGGCTCCCCCACCAGTACCGAGGAGGGGTCCCCCG |
| | | CTGGAAGTCCAACAAGCACTGAGGAAGGGTCCCCTGCCGGCTCCCCCACAAGTACC |
| | | GAAGAGGGCACAAGTGAGAGCGCCACTCCCGAGTCCGGGCCTGGCACCAGCACAGA |
| | | GCCTTCCGAGGGGTCCGCACCAGGTACCTCAGAGTCTGCTACCCCCGAGTCAGGGC |
| | | CAGGATCAGAGCCAGCCACCTCCGGGTCTGAGACACCCGGGACTTCCGAGAGTGCC |
| | | ACCCCTGAGTCCGGACCCGGGTCCGAGCCCGCCACTTCCGGCTCCGAAACTCCCGG |
| | | CACAAGCGAGAGCGCTACCCCAGAGTCAGGACCAGGAACATCTACAGAGCCCTCTG |
| | | AAGGCTCCGCTCCAGGGTCCCCAGCCGGCAGTCCCACTAGCACCGAGGAGGGAACC |
| | | TCTGAAAGCGCCACACCCGAATCAGGGCCAGGGTCTGAGCCTGCTACCAGCGGCAG |
| | | CGAGACACCAGGCACCTCTGAGTCCGCCACACCAGAGTCCGGACCCGGATCTCCCG |
| | | CTGGGAGCCCCACCTCCACTGAGGAGGGATCTCCTGCTGGCTCTCCAACATCTACT |
| | | GAGGAAGGTACCTCAACCGAGCCATCCGAGGGATCAGCTCCCGGCACCTCAGAGTC |
| | | GGCAACCCCGGAGTCTGGACCCGGAACTTCCGAAAGTGCCACACCAGAGTCCGGTC |
| | | CCGGGACTTCAGAATCAGCAACACCCGAGTCCGGCCCTGGGTCTGAACCCGCCACA |
| | | AGTGGTAGTGAGACACCAGGATCAGAACCTGCTACCTCAGGGTCAGAGACACCCGG |
| | | ATCTCCGGCAGGCTCACCAACCTCCACTGAGGAGGGCACCAGCACAGAACCAAGCG |
| | | AGGGCTCCGCACCCGGAACAAGCACTGAACCCAGTGAGGGTTCAGCACCCGGCTCT |
| | | GAGCCGGCCACAAGTGGCAGTGAGACACCCGGCACTTCAGAGAGTGCCACCCCCGA |
| | | GAGTGGCCCAGGCACTAGTACCGAGCCCTCTGAAGGCAGTGCGCCAX′GGCACAGC |
| | | CGAGGCCGCTAGCGCCAGCGGCATGTGGGAGCTGGAGAAGGACGTGTACGTGGTGG |
| | | AGGTGGACTGGACACCAGATGCCCCCGGCGAGACCGTGAACCTGACATGCGACACC |
| | | CCCGAGGAGGACGATATCACCTGGACATCTGATCAGAGGCACGGCGTGATCGGAAG |
| | | CGGCAAGACCCTGACAATCACCGTGAAGGAGTTCCTGGATGCCGGCCAGTACACAT |
| | | GTCACAAGGGCGGCGAGACCCTGTCCCACTCTCACCTGCTGCTGCACAAGAAGGAG |
| | | AACGGCATCTGGTCCACAGAGATCCTGAAGAACTTCAAGAATAAGACCTTTCTGAA |
| | | GTGCGAGGCCCCTAATTATAGCGGCCGGTTCACCTGTTCCTGGCTGGTGCAGAGAA |
| | | ACATGGACCTGAAGTTTAATATCAAGAGCTCCTCTAGCTCCCCAGATAGCCGGGCA |
| | | GTGACATGCGGAATGGCCAGCCTGTCCGCCGAGAAGGTGACCCTGGACCAGAGAGA |
| | | TTACGAGAAGTATTCTGTGAGCTGCCAGGAGGACGTGACATGTCCCACCGCCGAGG |
| | | AGACACTGCCTATCGAGCTGGCCCTGGAGGCCAGGCAGCAGAACAAGTACGAGAAT |
| | | TATTCCACCTCTTTCTTTATCCGCGACATCATCAAGCCAGATCCCCCTAAGAACCT |
| | | GCAGATGAAGCCCCTGAAGAATTCCCAGGTCGAGGTGTCTTGGGAGTACCCTGACA |
| | | GCTGGTCCACACCACACTCTTATTTCAGCCTGAAGTTCTTTGTGAGGATCCAGCGC |
| | | AAGAAGGAGAAGATGAAGGAGACCGAGGAGGGCTGCAATCAGAAGGGCGCCTTTCT |
| | | GGTGGAGAAGACATCCACCGAGGTGCAGTGCAAGGGAGGAAACGTGTGCGTGCAGG |
| | | CACAGGATCGGTACTATAATTCTAGCTGTTCCAAGTGGGCCTGCGTGCCTTGTCGG |
| | | GTGAGATCTGGCGGCGGCGGCTCTGGCGGCGGCGGCTCCGGCGGCGGCGGCTCCAG |
| | | AGTGATCCCCGTGAGCGGACCAGCAAGGTGCCTGTCCCAGAGCCGGAACCTGCTGA |
| | | AGACCACAGACGATATGGTGAAGACCGCCCGGGAGAAGCTGAAGCACTACTCTTGT |
| | | ACAGCCGAGGACATCGATCACGAGGACATCACCCGGGATCAGACCTCTACACTGAA |
| | | GACATGCCTGCCCCTGGAGCTGCACAAGAACGAGAGCTGTCTGGCCACCCGGGAGA |
| | | CAAGCTCCACCACAAGAGGCAGCTGCCTGCCCCCTCAGAAGACCTCCCTGATGATG |
| | | ACCCTGTGCCTGGGCTCTATCTACGAGGACCTGAAGATGTATCAGACCGAGTTCCA |
| | | GGCCATCAATGCCGCCCTGCAGAACCACAATCACCAGCAGATCATCCTGGACAAGG |
| | | GCATGCTGGTGGCCATCGATGAGCTGATGCAGAGCCTGAACCACAATGGCGAGACC |
| | | CTGAGGCAGAAGCCACCAGTGGGAGAGGCAGATCCTTACAGGGTGAAGATGAAGCT |
| | | GTGCATCCTGCTGCACGCCTTTTCCACCAGGGTGGTGACAATCAATCGCGTGATGG |
| | | GCTATCTGTCTAGCGCC |
| | | (wherein X′ is a polynucleotide sequence encoding a |
| | | release segment as set forth in Table 6 or 7) |
| Exemplified | Amino acid | ASHHHHHHHHGSPAGSPTSTEEGTSESATPESGPGTSTEPSEGSAPGSPAGSPTST |
| XTENylated | sequence | EEGTSTEPSEGSAPGTSTEPSEGSAPGTSESATPESGPGSEPATSGSETPGSEPAT |
| IL-12 | SEQ ID | SGSETPGSPAGSPTSTEEGTSESATPESGPGTSTEPSEGSAPGTSTEPSEGSAPGS |
| construct | NO: 2 | PAGSPTSTEEGTSTEPSEGSAPGTSTEPSEGSAPGTSESATPESGPGTSTEPSEGS |
| (N-terminal | | APGTSESATPESGPGSEPATSGSETPGTSTEPSEGSAPGTSTEPSEGSAPGTSESA |
| His-tag is | | TPESGPGTSESATPESGPGSPAGSPTSTEEGTSESATPESGPGSEPATSGSETPGT |
| optional) | | SESATPESGPGTSTEPSEGSAPGTSTEPSEGSAPGTSTEPSEGSAPGTSTEPSEGS |
| | | APGTSTEPSEGSAPGTSTEPSEGSAPGSPAGSPTSTEEGTSTEPSEGSAPGTSESA |
| | | TPESGPGSEPATSGSETPGTSESATPESGPGSEPATSGSETPGTSESATPESGPGT |
| | | STEPSEGSAPGTSESATPESGPGSPAGSPTSTEEGSPAGSPTSTEEGSPAGSPTST |
| | | EEGTSESATPESGPGTSTEPSEGSAPGTSESATPESGPGSEPATSGSETPGTSESA |
| | | TPESGPGSEPATSGSETPGTSESATPESGPGTSTEPSEGSAPGSPAGSPTSTEEGT |
| | | SESATPESGPGSEPATSGSETPGTSESATPESGPGSPAGSPTSTEEGSPAGSPTST |
| | | EEGTSTEPSEGSAPGTSESATPESGPGTSESATPESGPGTSESATPESGPGSEPAT |
| | | SGSETPGSEPATSGSETPGSPAGSPTSTEEGTSTEPSEGSAPGTSTEPSEGSAPGS |
| | | EPATSGSETPGTSESATPESGPGTSTEPSEGSAPXGTAEAASASGMWELEKDVYVV |
| | | EVDWTPDAPGETVNLTCDTPEEDDITWTSDQRHGVIGSGKTLTITVKEFLDAGQYT |
| | | CHKGGETLSHSHLLLHKKENGIWSTEILKNFKNKTFLKCEAPNYSGRFTCSWLVQR |
| | | NMDLKFNIKSSSSSPDSRAVTCGMASLSAEKVTLDQRDYEKYSVSCQEDVTCPTAE |
| | | ETLPIELALEARQQNKYENYSTSFFIRDIIKPDPPKNLQMKPLKNSQVEVSWEYPD |
| | | SWSTPHSYFSLKFFVRIQRKKEKMKETEEGCNQKGAFLVEKTSTEVQCKGGNVCVQ |
| | | AQDRYYNSSCSKWACVPCRVRSGGGGSGGGGSGGGGSRVIPVSGPARCLSQSRNLL |
| | | KTTDDMVKTAREKLKHYSCTAEDIDHEDITRDQTSTLKTCLPLELHKNESCLATRE |
| | | TSSTTRGSCLPPQKTSLMMTLCLGSIYEDLKMYQTEFQAINAALQNHNHQQIILDK |
| | | GMLVAIDELMQSLNHNGETLRQKPPVGEADPYRVKMKLCILLHAFSTRVVTINRVM |
| | | GYLSSA |
| | | (wherein X is an amino acid sequence (release segment) |
| | | as set forth in Table 6 or 7) |
| Reference | DNA | ATGTGGGAGCTGGAGAAGGACGTGTACGTGGTGGAGGTGGACTGGACACCAGATGC |
| construct | sequence | CCCCGGCGAGACCGTGAACCTGACATGCGACACCCCCGAGGAGGACGATATCACCT |
| (C-terminal | (SEQ ID | GGACATCTGATCAGAGGCACGGCGTGATCGGAAGCGGCAAGACCCTGACAATCACC |
| His-tag is | NO: 3) | GTGAAGGAGTTCCTGGATGCCGGCCAGTACACATGTCACAAGGGCGGCGAGACCCT |
| optional) | | GTCCCACTCTCACCTGCTGCTGCACAAGAAGGAGAACGGCATCTGGTCCACAGAGA |
| | | TCCTGAAGAACTTCAAGAATAAGACCTTTCTGAAGTGCGAGGCCCCTAATTATAGC |
| | | GGCCGGTTCACCTGTTCCTGGCTGGTGCAGAGAAACATGGACCTGAAGTTTAATAT |
| | | CAAGAGCTCCTCTAGCTCCCCAGATAGCCGGGCAGTGACATGCGGAATGGCCAGCC |
| | | TGTCCGCCGAGAAGGTGACCCTGGACCAGAGAGATTACGAGAAGTATTCTGTGAGC |
| | | TGCCAGGAGGACGTGACATGTCCCACCGCCGAGGAGACACTGCCTATCGAGCTGGC |
| | | CCTGGAGGCCAGGCAGCAGAACAAGTACGAGAATTATTCCACCTCTTTCTTTATCC |
| | | GCGACATCATCAAGCCAGATCCCCCTAAGAACCTGCAGATGAAGCCCCTGAAGAAT |
| | | TCCCAGGTCGAGGTGTCTTGGGAGTACCCTGACAGCTGGTCCACACCACACTCTTA |
| | | TTTCAGCCTGAAGTTCTTTGTGAGGATCCAGCGCAAGAAGGAGAAGATGAAGGAGA |
| | | CCGAGGAGGGCTGCAATCAGAAGGGCGCCTTTCTGGTGGAGAAGACATCCACCGAG |
| | | GTGCAGTGCAAGGGAGGAAACGTGTGCGTGCAGGCACAGGATCGGTACTATAATTC |
| | | TAGCTGTTCCAAGTGGGCCTGCGTGCCTTGTCGGGTGAGATCTGGCGGCGGCGGCT |
| | | CTGGCGGCGGCGGCTCCGGCGGCGGCGGCTCCAGAGTGATCCCCGTGAGCGGACCA |
| | | GCAAGGTGCCTGTCCCAGAGCCGGAACCTGCTGAAGACCACAGACGATATGGTGAA |
| | | GACCGCCCGGGAGAAGCTGAAGCACTACTCTTGTACAGCCGAGGACATCGATCACG |
| | | AGGACATCACCCGGGATCAGACCTCTACACTGAAGACATGCCTGCCCCTGGAGCTG |
| | | CACAAGAACGAGAGCTGTCTGGCCACCCGGGAGACAAGCTCCACCACAAGAGGCAG |
| | | CTGCCTGCCCCCTCAGAAGACCTCCCTGATGATGACCCTGTGCCTGGGCTCTATCT |
| | | ACGAGGACCTGAAGATGTATCAGACCGAGTTCCAGGCCATCAATGCCGCCCTGCAG |
| | | AACCACAATCACCAGCAGATCATCCTGGACAAGGGCATGCTGGTGGCCATCGATGA |
| | | GCTGATGCAGAGCCTGAACCACAATGGCGAGACCCTGAGGCAGAAGCCACCAGTGG |
| | | GAGAGGCAGATCCTTACAGGGTGAAGATGAAGCTGTGCATCCTGCTGCACGCCTTT |
| | | TCCACCAGGGTGGTGACAATCAATCGCGTGATGGGCTATCTGTCTAGCGCCCATCA |
| | | TCACCATCACCATCACCAT |
| Reference | Amino acid | MWELEKDVYVVEVDWTPDAPGETVNLTCDTPEEDDITWTSDQRHGVIGSGKTLTIT |
| construct | sequence | VKEFLDAGQYTCHKGGETLSHSHLLLHKKENGIWSTEILKNFKNKTFLKCEAPNYS |
| (C-terminal | SEQ ID | GRFTCSWLVQRNMDLKFNIKSSSSSPDSRAVTCGMASLSAEKVTLDQRDYEKYSVS |
| His-tag is | NO: 4 | CQEDVTCPTAEETLPIELALEARQQNKYENYSTSFFIRDIIKPDPPKNLQMKPLKN |
| optional) | | SQVEVSWEYPDSWSTPHSYFSLKFFVRIQRKKEKMKETEEGCNQKGAFLVEKTSTE |
| | | VQCKGGNVCVQAQDRYYNSSCSKWACVPCRVRSGGGGSGGGGSGGGGSRVIPVSGP |
| | | ARCLSQSRNLLKTTDDMVKTAREKLKHYSCTAEDIDHEDITRDQTSTLKTCLPLEL |
| | | HKNESCLATRETSSTTRGSCLPPQKTSLMMTLCLGSIYEDLKMYQTEFQAINAALQ |
| | | NHNHQQIILDKGMLVAIDELMQSLNHNGETLRQKPPVGEADPYRVKMKLCILLHAF |
| | | STRVVTINRVMGYLSSAHHHHHHHH |

The poly-histidine tag (His-tag), located at the C- or N-terminus of each exemplified fusion protein, as shown hereinabove in Table B, is optional.

### Example 8: IL12 Activity Assay

HEK-Blue IL12 reporter cells were purchased from InvivoGen and cultured at 37° C., 50% CO₂ in a culture media consisting of DMEM, 4.5 g/l glucose, 2 mM L-glutamine, 10% (v/v) heat-inactivated fetal bovine serum, 100 U/ml penicillin, 100 μg/ml streptomycin, 100 μg/ml Normocin, 1×HEK-Blue Selection. For the IL12 activity assay, a test medium was prepared as described in the immediately preceding sentence but without Normocin and Selection antibiotics. The test medium and 1×PBS were warmed to 37° C. in a water bath. Cells were dislodged from the flask by washing the flask with the pre-warmed PBS, followed by a centrifugation at 300×g (1200 rpm) for 5 mins at room temperature, determination of cell viability, and a resuspension of the cell pellet in the test medium to 0.833×10e6 cells/mL. Ninety microliters (90 μL) of the cells were aliquoted into each well of a 96-well flat-clear-bottom plate (Costar, cat #3595). IL12 test articles were prepared at 10×concentration in the test medium with 17 nM being the highest concentration, followed by a serial 10-fold dilution to 1.7 μM. Then, 10 uL of the 10×solution were added to the 90 μL of cells, and the plate was incubated for 24h. The next day, a QuantiBlue solution, the detection reagent for secreted embryonic alkaline phosphatase (SEAP), was prepared by diluting QB reagent and QB buffer in room temperature MilliQ water to 10% (v/v) concentration each. The mixture was incubated at room temperature for 10 minutes. Subsequently, 180 μL were aliquoted to each well of a 96-well flat bottom tissue culture plate, and to each well was added 20 μL of the supernatant. The plate was incubated at 37° C., 5% CO₂ for 6h. At different incubation time intervals (15 min, 30 min, 1h, 2h, 3h), a microplate reader was used to measure the optical density (O.D.) at 650 nm. The results were analyzed by Excel software and presented here from the 3 hr timepoint.

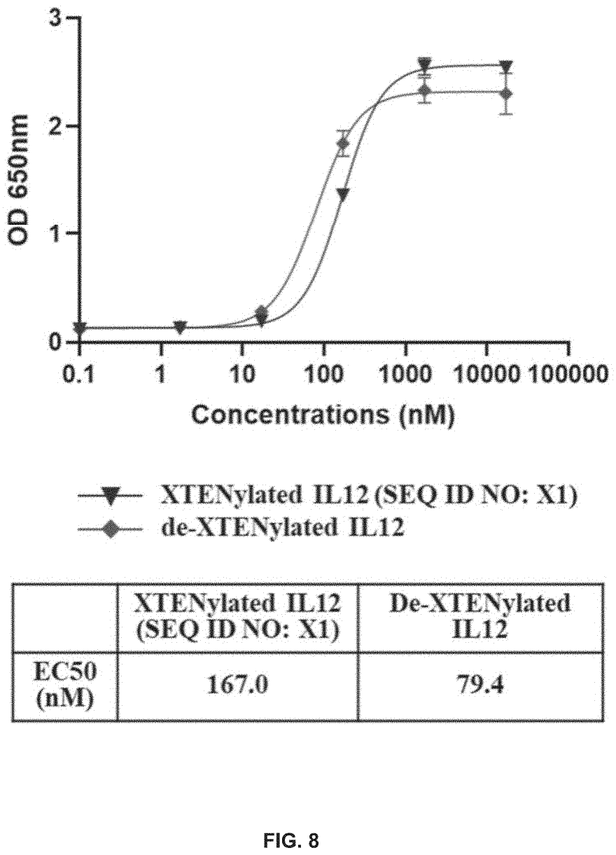

As shown in FIG. 8, IL-12 reporter cells that produce secreted embryonic alkaline phosphatase (SEAP) in response to IL-12-induced STAT4 activation were treated with increasing concentrations of the IL-12 test articles for 24h. The levels of SEAP in the supernatant were measured using a QuantiBlue solution, and the plate was read at optical density of 650 nm. The XTENylated IL12 (SEQ ID NO: 2) composition curve (triangle) is shifted at least 2×relative to the corresponding de-XTENylated IL12 composition curve (diamond), indicating a masking effect of the XTEN that reduces cytokine activity.

### Example 9: IL12 Receptor Binding Assay

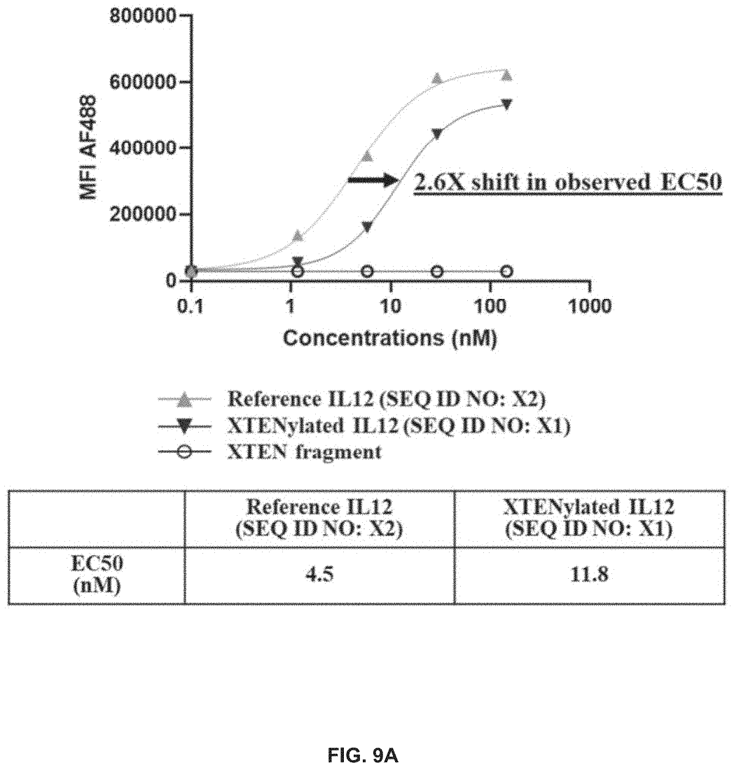

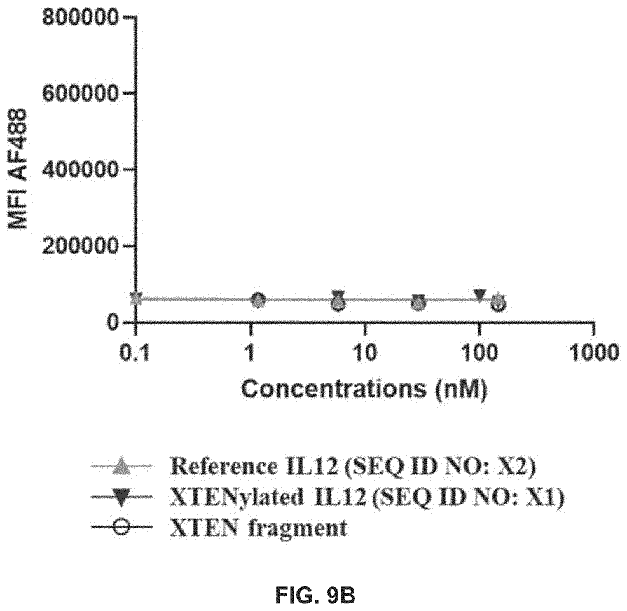

HEK-Blue IL-12 reporter cells (Invivogen, as described in Example 8) that express the human IL-12 receptor were used to assess binding of the IL12 constructs to the IL-12 receptor. Increasing concentrations of an exemplified “XTENylated IL12” construct (SEQ ID NO: 2) (1 μM) containing a recombinant single chain mouse IL12 with an N-terminal his-tag plus an XTEN sequence followed by a release segment sequence were incubated with 50,000 293HEK-IL-12 reporter cells that were subsequently washed and surface bound IL12 monitored by flow cytometry using a fluorescent-labelled anti-His-tag antibody for detection. Binding by the XTENylated IL12 was compared to binding of the reference IL-12 construct (SEQ ID NO:4) that contained a recombinant single chain mouse IL-12 and a C-terminal His-tag. Due to release of the His-tag from the XTENylated IL-12 following its activation with human matrix metallopeptidase 9 (MMP9), we were unable to assess IL-12 binding of its activated form in this assay. The XTEN fragment released by MMP9 cleavage retained the His-tag and was used as a specificity control for binding. As shown in FIGS. 9A-9B, the XTEN, when present in the fusion protein masked the cytokine binding to its corresponding IL12 receptor. The XTENylated IL-12 exhibited a binding affinity that is reduced compared with the corresponding binding activity of the IL12 when not linked to the XTEN, as characterized by an increase in the half maximal effective concentration (EC50).

### Example 10: Exemplary Xtenylated IL12 Constructs

In certain exemplary embodiments, XTENylated IL12 constructs were created using IL12 subunits that have been Xtenylated four times. The table below provides the nucleic acid and amino acid sequences of exemplary IL12 p35 subunit that has been Xtenylated and an IL12 p40 subunit that has been Xtenylated.

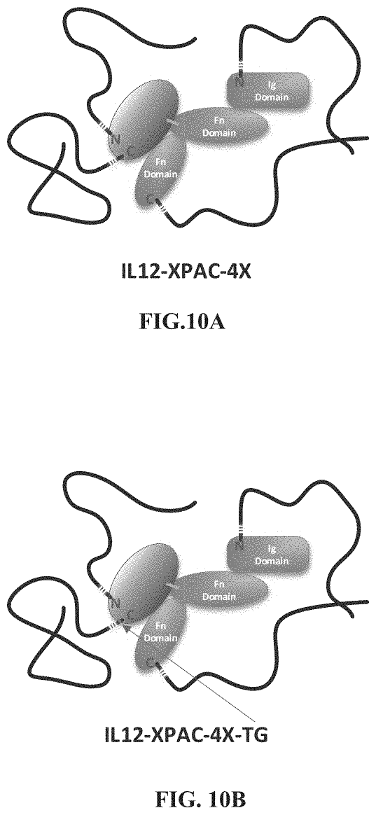

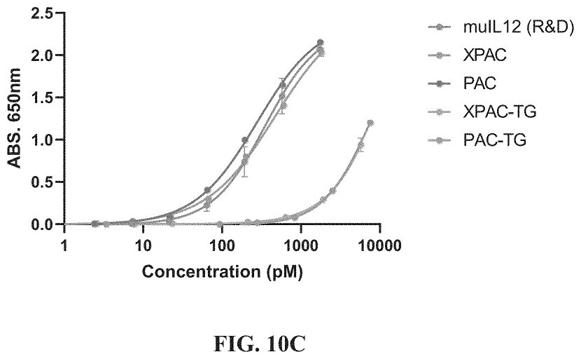

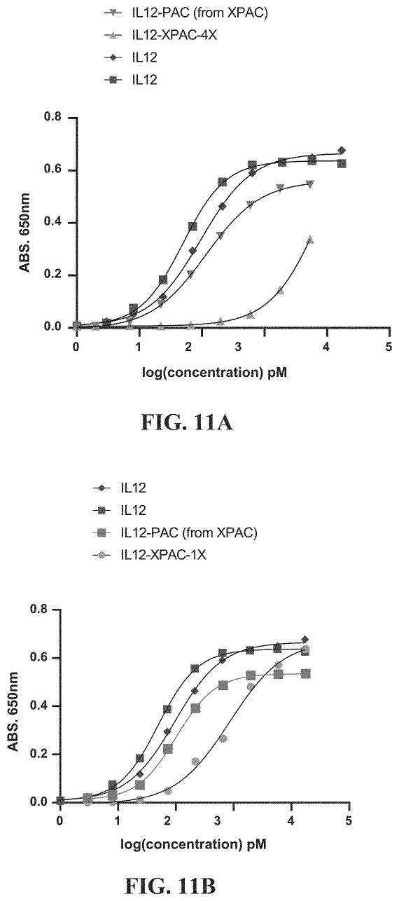

FIGS. 10A and 10B show a schematic representation of the above two constructs. HEK Blue IL12 activity assays were performed substantially as described in Example 9 above. The data from those assays is collated in FIG. 10C and represented in the Table 19 below:

TABLE 19
IL12 Activity Reported Using HEK Blue Assay

| | EC50 | Masking |
|---|---|---|
| muIL12 | 25 | n/a |
| (AP2551 XPAC) | 7117 | 430 |
| (AP2551 PAC) | 17 | — |
| (AP2552 XPAC with TG tag) | 2746 | 95 |
| (AP2552 PAC with TG tag) | 29 | — |

These data clearly show that PACs generated have equivalent activity to recombinant muIL12 as expected for a heterodimeric preparation and that the XTENylation of the IL12 resulted in a binding affinity that is reduced compared with the corresponding binding activity of the IL12 when not linked to the XTEN, as characterized by an increase in the half maximal effective concentration (EC50). As such, this data show that IL12-XPAC-4×constructs exhibit sufficient masking and activity to be comparable to that of naked IL12. Moreover, presence of a transglutaminase tag does not influence IL12 activity.

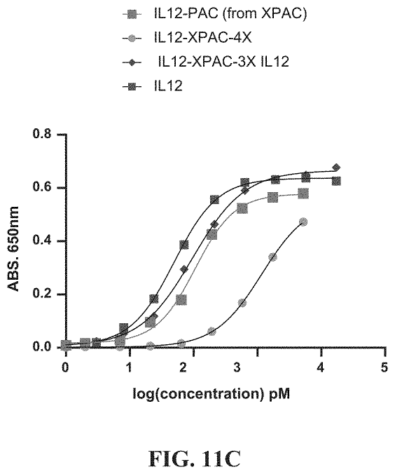

In a further analysis, the effect of 1 (AP2450), 3 (AP2447), and 4 (AP2446) XTENs on IL12 compared (FIGS. 11 A-C and Table 20).

| | AP2446-XPAC | AP2447-XPAC | AP2450-XPAC |
|---|---|---|---|
| | (4XTEN) | (3XTEN) | (1XTEN) |
| | 9024 | 849.5 | 1171 |
| | AP2446-PAC | AP2447-PAC | AP2450-PAC |
| EC50 (pM) | 115.2 | 102 | 106.1 |
| Fold Masking | 78 | 8 | 11 |

Of the data generated, it was seen that all XTENs contribute to masking and that increasing XTEN at a single site does not provide additional benefit but use of a dual plasmid format for expression offers additional XTEN addition benefits. The most preferred constructs: AP2446, AP2450, AP2407, were selected for further study.

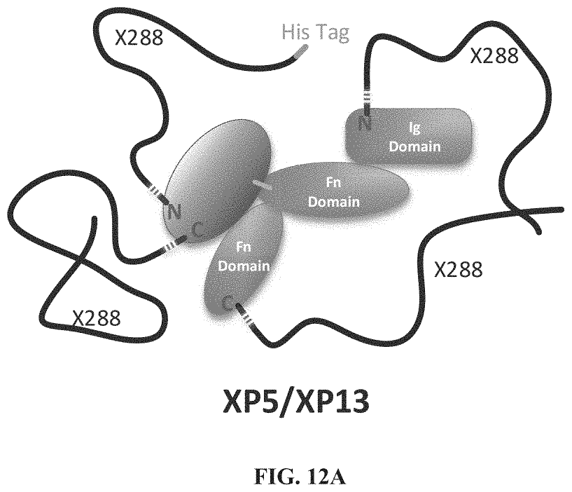

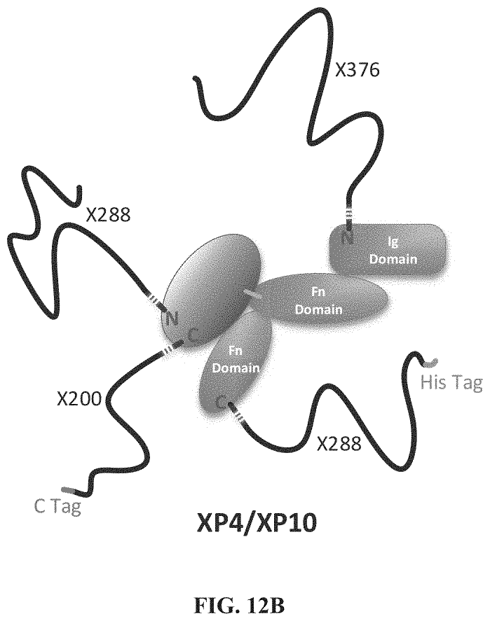

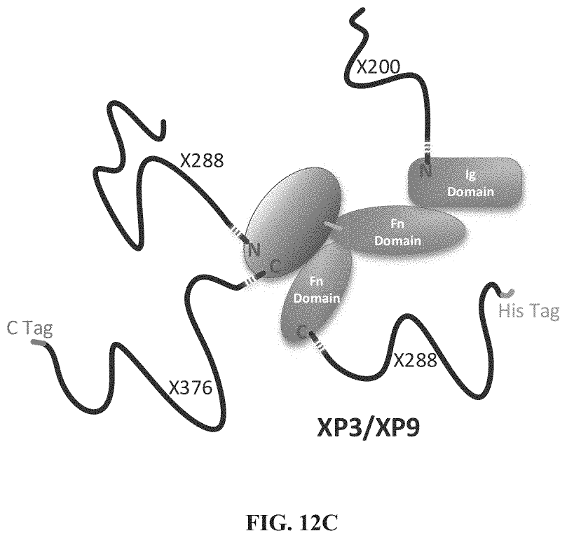

In the next iteration, the IL12-XPAC-4×construct was redesigned to explore designs for each of purification and analytics of the IL12 heterodimers. The design of three constructs is shown in the following table and a schematic of the constructs is shown in FIG. 12A (IL12-XPAC-4X.1 comprised of XP5/XP13 sequence shown in Table 22), 12B (IL12-XPAC-4X.2 comprised of XP4/XP10 sequence shown in Table 22) and 12C (IL12-XPAC-4X.3 comprised of XP3/XP9 sequence shown in Table 22) as schematics and described in the Table 21 below:

TABLE 21
Features of Three Exemplary IL12-XPACs each comprising 4 XTEN sequences

| | | | | | Subunit | |
|---|---|---|---|---|---|---|
| | | | | Total | Size | Total Protein |
| Constructs | Subunits | Format | #XTENs | XTEN AA | (kDa) | Size (kDa) |
| IL12-XPAC-4X.1 | | | 4 | 1152 | | 172 |
| | XTEN | X288-P40-X288 | 2 | | 93 | |
| | P40 | | | | | |
| | XTEN | X288-P35-X288 | 2 | | 79 | |
| | P35 | | | | | |
| IL12-XPAC-4X.2 | | | 4 | 1152 | | 172 |
| | AC3244 | X376-P40-X288 | 2 | | 101 | |
| | AC3247 | X376-P35-X200 | 2 | | 71 | |
| IL12-XPAC-4X.3 | | | 4 | 1152 | | 172 |
| | AC3245 | X200-P40-X288 | 2 | | 85 | |
| | AC3246 | X288-P35-X376 | 2 | | 87 | |

TABLE 22
Sequences of exemplary xtenylated subunits for XPACs shown in Table 21

| XP No. | DNA Sequence | Protein Sequence | Domain |
|---|---|---|---|
| XP01 | GGTTCTCCAGCCGGGTCCCCAACTTCGACCGAGGAAGGGACCTCC | GSPAGSPTSTEEGTSE | P40 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGRVIPVS | |
| | CTGACCGGCCCTGGCACAGCCGAGGCCGCTAGCGCCAGCGGCAGA | GPARCLSQSRNLLKTT | |
| | GTGATCCCCGTGAGCGGACCAGCAAGGTGCCTGTCCCAGAGCCGG | DDMVKTAREKLKHYSC | |
| | AACCTGCTGAAGACCACAGACGATATGGTGAAGACCGCCCGGGAG | TAEDIDHEDITRDQTS | |
| | AAGCTGAAGCACTACTCTTGTACAGCCGAGGACATCGATCACGAG | TLKTCLPLELHKNESC | |
| | GACATCACCCGGGATCAGACCTCTACACTGAAGACATGCCTGCCC | LATRETSSTTRGSCLP | |
| | CTGGAGCTGCACAAGAACGAGAGCTGTCTGGCCACCCGGGAGACA | PQKTSLMMTLCLGSIY | |
| | AGCTCCACCACAAGAGGCAGCTGCCTGCCCCCTCAGAAGACCTCC | EDLKMYQTEFQAINAA | |
| | CTGATGATGACCCTGTGCCTGGGCTCTATCTACGAGGACCTGAAG | LQNHNHQQIILDKGML | |
| | ATGTATCAGACCGAGTTCCAGGCCATCAATGCCGCCCTGCAGAAC | VAIDELMQSLNHNGET | |
| | CACAATCACCAGCAGATCATCCTGGACAAGGGCATGCTGGTGGCC | LRQKPPVGEADPYRVK | |
| | ATCGATGAGCTGATGCAGAGCCTGAACCACAATGGCGAGACCCTG | MKLCILLHAFSTRVVT | |
| | AGGCAGAAGCCACCAGTGGGAGAGGCAGATCCTTACAGGGTGAAG | INRVMGYLSSAGTAEA | |
| | ATGAAGCTGTGCATCCTGCTGCACGCCTTTTCCACCAGGGTGGTG | ASASGVLQSPGTSESA | |
| | ACAATCAATCGCGTGATGGGCTATCTGTCTAGCGCCGGCACAGCC | TPESGPGSEPATSGSE | |
| | GAGGCCGCTAGCGCCAGCGGCGTGCTGCAGAGCCCAGGTACCTCA | TPGTSESATPESGPGS | |
| | GAGTCTGCTACCCCCGAGTCAGGGCCAGGATCAGAGCCAGCCACC | EPATSGSETPGTSESA | |
| | TCCGGGTCTGAGACACCCGGGACTTCCGAGAGTGCCACCCCTGAG | TPESGPGTSTEPSEGS | |
| | TCCGGACCCGGGTCCGAGCCCGCCACTTCCGGCTCCGAAACTCCC | APGSPAGSPTSTEEGT | |
| | GGCACAAGCGAGAGCGCTACCCCAGAGTCAGGACCAGGAACATCT | SESATPESGPGSEPAT | |
| | ACAGAGCCCTCTGAAGGCTCCGCTCCAGGGTCCCCAGCCGGCAGT | SGSETPGTSESATPES | |
| | CCCACTAGCACCGAGGAGGGAACCTCTGAAAGCGCCACACCCGAA | GPGSPAGSPTSTEEGS | |
| | TCAGGGCCAGGGTCTGAGCCTGCTACCAGCGGCAGCGAGACACCA | PAGSPTSTEEGTSTEP | |
| | GGCACCTCTGAGTCCGCCACACCAGAGTCCGGACCCGGATCTCCC | SEGSAPGTSESATPES | |
| | GCTGGGAGCCCCACCTCCACTGAGGAGGGATCTCCTGCTGGCTCT | GPGTSESATPESGPGT | |
| | CCAACATCTACTGAGGAAGGTACCTCAACCGAGCCATCCGAGGGA | SESATPESGPGSEPAT | |
| | TCAGCTCCCGGCACCTCAGAGTCGGCAACCCCGGAGTCTGGACCC | SGSETPGSEPATSGSE | |
| | GGAACTTCCGAAAGTGCCACACCAGAGTCCGGTCCCGGGACTTCA | TPGSPAGSPTSTEEGT | |
| | GAATCAGCAACACCCGAGTCCGGCCCTGGGTCTGAACCCGCCACA | STEPSEGSAPGTSTEP | |
| | AGTGGTAGTGAGACACCAGGATCAGAACCTGCTACCTCAGGGTCA | SEGSAPGSEPATSGSE | |
| | GAGACACCCGGATCTCCGGCAGGCTCACCAACCTCCACTGAGGAG | TPGTSESATPESGPGT | |
| | GGCACCAGCACAGAACCAAGCGAGGGCTCCGCACCCGGAACAAGC | STEPSEGSAPEIVLTQ | |
| | ACTGAACCCAGTGAGGGTTCAGCACCCGGCTCTGAGCCGGCCACA | SPGTLSLSPGERATLS | |
| | AGTGGCAGTGAGACACCCGGCACTTCAGAGAGTGCCACCCCCGAG | CRASQSVSSSFLAWYQ | |
| | AGTGGCCCAGGCACTAGTACCGAGCCCTCTGAAGGCAGTGCGCCA | QKPGQAPRLLIYYASS | |
| | GAGATCGTCCTGACCCAATCCCCCGGGACCCTCAGCCTGAGCCCA | RATGIPDRFSGSGSGT | |
| | GGCGAGCGTGCCACTTTGAGCTGTCGTGCATCACAGAGTGTGAGT | DFTLTISRLEPEDFAV | |
| | TCCTCATTCCTGGCTTGGTACCAGCAAAAGCCCGGTCAGGCCCCG | YYCQQTGRIPPTFGQG | |
| | AGACTTTTGATTTACTATGCTTCCAGCCGCGCTACCGGGATCCCA | TKVEIKGATPPETGAE | |
| | GATAGATTTTCTGGGAGCGGTTCTGGTACCGATTTCACTCTGACC | TESPGETTGGSAESEP | |
| | ATCTCTAGACTCGAACCAGAAGACTTTGCAGTATATTACTGCCAA | PGEGEVQLLESGGGLV | |
| | CAGACCGGTCGGATCCCTCCAACTTTCGGACAGGGTACCAAGGTT | QPGGSLRLSCAASGFT | |
| | GAGATCAAGGGTGCAACGCCTCCGGAGACTGGTGCTGAAACTGAG | FSSFSMSWVRQAPGKG | |
| | TCCCCGGGCGAGACGACCGGTGGCTCTGCTGAATCCGAACCACCG | LEWVSSISGSSGTTYY | |
| | GGCGAAGGCGAGGTCCAGCTGTTGGAGAGCGGCGGTGGACTCGTG | ADSVKGRFTISRDNSK | |
| | CAGCCGGGCGGTTCACTTCGTCTCAGTTGTGCTGCCTCAGGCTTC | NTLYLQMNSLRAEDTA | |
| | ACCTTTAGCTCATTCTCAATGAGTTGGGTGAGACAGGCGCCCGGC | VYYCAKPFPYFDYWGQ | |
| | AAGGGCCTTGAGTGGGTTAGTTCCATTTCCGGCTCCAGCGGCACT | GTLVTVSSGTAEAASA | |
| | ACCTACTATGCCGACTCAGTCAAAGGTAGATTTACCATCTCCCGC | SGEAGRSANHTPAGLT | |
| | GATAACTCTAAGAACACCCTGTACCTGCAGATGAACTCCCTCAGG | GPGSPAGSPTSTEEGT | |
| | GCAGAGGATACCGCCGTGTACTATTGCGCGAAGCCCTTCCCATAC | SESATPESGPGSEPAT | |
| | TTCGACTACTGGGGTCAGGGCACCCTGGTCACTGTCAGTTCCGGC | SGSETPGTSESATPES | |
| | ACAGCCGAGGCCGCTAGCGCCAGCGGCGAGGCCGGCCGGAGCGCC | GPGTSTEPSEGSAPGT | |
| | AACCACACCCCCGCCGGCCTGACCGGCCCTGGTTCTCCTGCTGGC | STEPSEGSAPGTSTEP | |
| | TCCCCCACCTCAACAGAAGAGGGGACAAGCGAAAGCGCTACGCCT | SEGSAPGTSTEPSEGS | |
| | GAGAGTGGCCCTGGCTCTGAGCCAGCCACCTCCGGCTCTGAAACC | APGTSTEPSEGSAPGT | |
| | CCTGGCACTAGTGAGTCTGCCACGCCTGAGTCCGGACCCGGGACC | STEPSEGSAPGSPAGS | |
| | TCTACTGAGCCCTCGGAGGGGAGCGCTCCTGGCACGAGTACAGAA | PTSTEEGTSTEPSEGS | |
| | CCTTCCGAAGGAAGTGCACCGGGCACAAGCACCGAGCCTTCCGAA | APGTSESATPESGPGS | |
| | GGCTCTGCTCCCGGAACCTCTACCGAACCCTCTGAAGGGTCTGCA | EPATSGSETPGTSESA | |
| | CCCGGCACGAGCACCGAACCCAGCGAAGGGTCAGCGCCTGGGACC | TPESGPGSEPATSGSE | |
| | TCAACAGAGCCCTCGGAAGGATCAGCGCCTGGAAGCCCTGCAGGG | TPGTSESATPESGPGT | |
| | AGTCCAACTTCCACGGAAGAAGGAACGTCTACAGAGCCATCAGAG | STEPSEGSAPGTSESA | |
| | GGGTCCGCACCAGGTACCAGCGAATCCGCTACTCCCGAATCTGGC | TPESGPGSPAGSPTST | |
| | CCTGGGTCCGAACCTGCCACCTCCGGCTCTGAAACTCCAGGGACC | EEGSPAGSPTSTEEGS | |
| | TCCGAATCTGCCACACCCGAGAGCGGCCCTGGCTCCGAGCCCGCA | PAGSPTSTEEGTSESA | |
| | ACATCTGGCAGCGAGACACCTGGCACCTCCGAGAGCGCAACACCC | TPESGPGTSTEPSEGS | |
| | GAGAGCGGCCCTGGCACCAGCACCGAGCCATCCGAGGGATCCGCC | AP (SEQ ID NO: | |
| | CCAGGCACTTCTGAGTCAGCCACACCCGAAAGCGGACCAGGATCA | 849) | |
| | CCCGCTGGCTCCCCCACCAGTACCGAGGAGGGGTCCCCCGCTGGA | | |
| | AGTCCAACAAGCACTGAGGAAGGGTCCCCTGCCGGCTCCCCCACA | | |
| | AGTACCGAAGAGGGCACAAGTGAGAGCGCCACTCCCGAGTCCGGG | | |
| | CCTGGCACCAGCACAGAGCCTTCCGAGGGGTCCGCACCA (SEQ | | |
| | ID NO: 831) | | |
| XP02 | ATGTGGGAGCTGGAGAAGGACGTGTACGTGGTGGAGGTGGACTGG | MWELEKDVYVVEVDWT | P40 |
| | ACACCAGATGCCCCCGGCGAGACCGTGAACCTGACATGCGACACC | PDAPGETVNLTCDTPE | |
| | CCCGAGGAGGACGATATCACCTGGACATCTGATCAGAGGCACGGC | EDDITWTSDQRHGVIG | |
| | GTGATCGGAAGCGGCAAGACCCTGACAATCACCGTGAAGGAGTTC | SGKTLTITVKEFLDAG | |
| | CTGGATGCCGGCCAGTACACATGTCACAAGGGCGGCGAGACCCTG | QYTCHKGGETLSHSHL | |
| | TCCCACTCTCACCTGCTGCTGCACAAGAAGGAGAACGGCATCTGG | LLHKKENGIWSTEILK | |
| | TCCACAGAGATCCTGAAGAACTTCAAGAATAAGACCTTTCTGAAG | NFKNKTFLKCEAPNYS | |
| | TGCGAGGCCCCTAATTATAGCGGCCGGTTCACCTGTTCCTGGCTG | GRFTCSWLVQRNMDLK | |
| | GTGCAGAGAAACATGGACCTGAAGTTTAATATCAAGAGCTCCTCT | FNIKSSSSSPDSRAVT | |
| | AGCTCCCCAGATAGCCGGGCAGTGACATGCGGAATGGCCAGCCTG | CGMASLSAEKVTLDQR | |
| | TCCGCCGAGAAGGTGACCCTGGACCAGAGAGATTACGAGAAGTAT | DYEKYSVSCQEDVTCP | |
| | TCTGTGAGCTGCCAGGAGGACGTGACATGTCCCACCGCCGAGGAG | TAEETLPIELALEARQ | |
| | ACACTGCCTATCGAGCTGGCCCTGGAGGCCAGGCAGCAGAACAAG | QNKYENYSTSFFIRDI | |
| | TACGAGAATTATTCCACCTCTTTCTTTATCCGCGACATCATCAAG | IKPDPPKNLQMKPLKN | |
| | CCAGATCCCCCTAAGAACCTGCAGATGAAGCCCCTGAAGAATTCC | SQVEVSWEYPDSWSTP | |
| | CAGGTCGAGGTGTCTTGGGAGTACCCTGACAGCTGGTCCACACCA | HSYFSLKFFVRIQRKK | |
| | CACTCTTATTTCAGCCTGAAGTTCTTTGTGAGGATCCAGCGCAAG | EKMKETEEGCNQKGAF | |
| | AAGGAGAAGATGAAGGAGACCGAGGAGGGCTGCAATCAGAAGGGC | LVEKTSTEVQCKGGNV | |
| | GCCTTTCTGGTGGAGAAGACATCCACCGAGGTGCAGTGCAAGGGA | CVQAQDRYYNSSCSKW | |
| | GGAAACGTGTGCGTGCAGGCACAGGATCGGTACTATAATTCTAGC | ACVPCRVRSGTAEAAS | |
| | TGTTCCAAGTGGGCCTGCGTGCCTTGTCGGGTGAGATCTGGCACA | ASGEAGRSANHTPAGL | |
| | GCCGAGGCCGCTAGCGCCAGCGGCGAGGCCGGCCGGAGCGCCAAC | TGPGSPAGSPTSTEEG | |
| | CACACCCCCGCCGGCCTGACCGGCCCTGGTTCTCCAGCCGGGTCC | TSESATPESGPGTSTE | |
| | CCAACTTCGACCGAGGAAGGGACCTCCGAGTCAGCTACCCCGGAG | PSEGSAPGSPAGSPTS | |
| | TCCGGTCCTGGCACCTCCACCGAACCATCGGAGGGCAGCGCCCCT | TEEGTSTEPSEGSAPG | |
| | GGGAGCCCTGCCGGGAGCCCTACAAGCACCGAAGAGGGCACCAGT | TSTEPSEGSAPGTSES | |
| | ACAGAGCCAAGTGAGGGGAGCGCCCCTGGTACTAGTACTGAACCA | ATPESGPGSEPATSGS | |
| | TCCGAGGGGTCAGCTCCAGGCACGAGTGAGTCCGCTACCCCCGAG | ETPGSEPATSGSETPG | |
| | AGCGGACCGGGCTCAGAGCCCGCCACGAGTGGCAGTGAAACTCCA | SPAGSPTSTEEGTSES | |
| | GGCTCAGAACCCGCCACTAGTGGGTCAGAGACTCCAGGCAGCCCT | ATPESGPGTSTEPSEG | |
| | GCCGGATCCCCTACGTCCACCGAGGAGGGAACATCTGAGTCCGCA | SAPGTSTEPSEGSAPG | |
| | ACACCCGAATCCGGTCCAGGCACCTCCACGGAACCTAGTGAAGGC | SPAGSPTSTEEGTSTE | |
| | TCGGCACCAGGTACAAGCACCGAACCTAGCGAGGGCAGCGCTCCC | PSEGSAPGTSTEPSEG | |
| | GGCAGCCCTGCCGGCAGCCCAACCTCAACTGAGGAGGGCACCAGT | SAPGTSESATPESGPG | |
| | ACTGAGCCCAGCGAGGGATCAGCACCTGGCACCAGCACCGAACCT | TSTEPSEGSAPGTSES | |
| | AGCGAGGGGAGCGCCCCTGGGACTAGCGAGTCAGCTACACCAGAG | ATPESGPGSEPATSGS | |
| | AGCGGGCCTGGAACTTCTACCGAACCCAGTGAGGGATCCGCTCCA | ETPGTSTEPSEGSAPG | |
| | GGCACCTCCGAATCCGCAACCCCCGAATCCGGACCTGGCTCAGAG | TSTEPSEGSAPGTSES | |
| | CCCGCCACCAGCGGGAGCGAAACCCCTGGCACATCCACCGAGCCT | ATPESGPGTSESATPE | |
| | AGCGAAGGGTCCGCACCCGGCACCAGTACAGAGCCTAGCGAGGGA | SGPGSPAGSPTSTEEG | |
| | TCAGCACCTGGCACCAGTGAATCTGCTACACCAGAGAGCGGCCCT | TSESATPESGPGSEPA | |
| | GGAACCTCCGAGTCCGCTACCCCCGAGAGCGGGCCAGGTTCTCCT | TSGSETPGTSESATPE | |
| | GCTGGCTCCCCCACCTCAACAGAAGAGGGGACAAGCGAAAGCGCT | SGPGTSTEPSEGSAPG | |
| | ACGCCTGAGAGTGGCCCTGGCTCTGAGCCAGCCACCTCCGGCTCT | TSTEPSEGSAPGTSTE | |
| | GAAACCCCTGGCACTAGTGAGTCTGCCACGCCTGAGTCCGGACCC | PSEGSAPGTSTEPSEG | |
| | GGGACCTCTACTGAGCCCTCGGAGGGGAGCGCTCCTGGCACGAGT | SAPGTSTEPSEGSAPG | |
| | ACAGAACCTTCCGAAGGAAGTGCACCGGGCACAAGCACCGAGCCT | TSTEPSEGSAPGSPAG | |
| | TCCGAAGGCTCTGCTCCCGGAACCTCTACCGAACCCTCTGAAGGG | SPTSTEEGTSTEPSEG | |
| | TCTGCACCCGGCACGAGCACCGAACCCAGCGAAGGGTCAGCGCCT | SAPGTSESATPESGPG | |
| | GGGACCTCAACAGAGCCCTCGGAAGGATCAGCGCCTGGAAGCCCT | SEPATSGSETPGTSES | |
| | GCAGGGAGTCCAACTTCCACGGAAGAAGGAACGTCTACAGAGCCA | ATPESGPGSEPATSGS | |
| | TCAGAGGGGTCCGCACCAGGTACCAGCGAATCCGCTACTCCCGAA | ETPGTSESATPESGPG | |
| | TCTGGCCCTGGGTCCGAACCTGCCACCTCCGGCTCTGAAACTCCA | TSTEPSEGSAPGTSES | |
| | GGGACCTCCGAATCTGCCACACCCGAGAGCGGCCCTGGCTCCGAG | ATPESGPGSPAGSPTS | |
| | CCCGCAACATCTGGCAGCGAGACACCTGGCACCTCCGAGAGCGCA | TEEGSPAGSPTSTEEG | |
| | ACACCCGAGAGCGGCCCTGGCACCAGCACCGAGCCATCCGAGGGA | SPAGSPTSTEEGTSES | |
| | TCCGCCCCAGGCACTTCTGAGTCAGCCACACCCGAAAGCGGACCA | ATPESGPGTSTEPSEG | |
| | GGATCACCCGCTGGCTCCCCCACCAGTACCGAGGAGGGGTCCCCC | SAPGTSESATPESGPG | |
| | GCTGGAAGTCCAACAAGCACTGAGGAAGGGTCCCCTGCCGGCTCC | SEPATSGSETPGTSES | |
| | CCCACAAGTACCGAAGAGGGCACAAGTGAGAGCGCCACTCCCGAG | ATPESGPGSEPATSGS | |
| | TCCGGGCCTGGCACCAGCACAGAGCCTTCCGAGGGGTCCGCACCA | ETPGTSESATPESGPG | |
| | GGTACCTCAGAGTCTGCTACCCCCGAGTCAGGGCCAGGATCAGAG | TSTEPSEGSAPGSPAG | |
| | CCAGCCACCTCCGGGTCTGAGACACCCGGGACTTCCGAGAGTGCC | SPTSTEEGTSESATPE | |
| | ACCCCTGAGTCCGGACCCGGGTCCGAGCCCGCCACTTCCGGCTCC | SGPGSEPATSGSETPG | |
| | GAAACTCCCGGCACAAGCGAGAGCGCTACCCCAGAGTCAGGACCA | TSESATPESGPGSPAG | |
| | GGAACATCTACAGAGCCCTCTGAAGGCTCCGCTCCAGGGTCCCCA | SPTSTEEGSPAGSPTS | |
| | GCCGGCAGTCCCACTAGCACCGAGGAGGGAACCTCTGAAAGCGCC | TEEGTSTEPSEGSAPG | |
| | ACACCCGAATCAGGGCCAGGGTCTGAGCCTGCTACCAGCGGCAGC | TSESATPESGPGTSES | |
| | GAGACACCAGGCACCTCTGAGTCCGCCACACCAGAGTCCGGACCC | ATPESGPGTSESATPE | |
| | GGATCTCCCGCTGGGAGCCCCACCTCCACTGAGGAGGGATCTCCT | SGPGSEPATSGSETPG | |
| | GCTGGCTCTCCAACATCTACTGAGGAAGGTACCTCAACCGAGCCA | SEPATSGSETPGSPAG | |
| | TCCGAGGGATCAGCTCCCGGCACCTCAGAGTCGGCAACCCCGGAG | SPTSTEEGTSTEPSEG | |
| | TCTGGACCCGGAACTTCCGAAAGTGCCACACCAGAGTCCGGTCCC | SAPGTSTEPSEGSAPG | |
| | GGGACTTCAGAATCAGCAACACCCGAGTCCGGCCCTGGGTCTGAA | SEPATSGSETPGTSES | |
| | CCCGCCACAAGTGGTAGTGAGACACCAGGATCAGAACCTGCTACC | ATPESGPGTSTEPSEG | |
| | TCAGGGTCAGAGACACCCGGATCTCCGGCAGGCTCACCAACCTCC | SAP (SEQ ID NO: | |
| | ACTGAGGAGGGCACCAGCACAGAACCAAGCGAGGGCTCCGCACCC | 850) | |
| | GGAACAAGCACTGAACCCAGTGAGGGTTCAGCACCCGGCTCTGAG | | |
| | CCGGCCACAAGTGGCAGTGAGACACCCGGCACTTCAGAGAGTGCC | | |
| | ACCCCCGAGAGTGGCCCAGGCACTAGTACCGAGCCCTCTGAAGGC | | |
| | AGTGCGCCA (SEQ ID NO: 832) | | |
| XP03 | GGTTCTCCAGCCGGGTCCCCAACTTCGACCGAGGAAGGGACCTCC | GSPAGSPTSTEEGTSE | P40 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPEAGRSANH | |
| | GAGTCAGCTACACCAGAGGCCGGCCGGAGCGCCAACCACACCCCC | TPAGLTGPGTAEAASA | |
| | GCCGGCCTGACCGGCCCTGGCACAGCCGAGGCCGCTAGCGCCAGC | SGMWELEKDVYVVEVD | |
| | GGCATGTGGGAGCTGGAGAAGGACGTGTACGTGGTGGAGGTGGAC | WTPDAPGETVNLTCDT | |
| | TGGACACCAGATGCCCCCGGCGAGACCGTGAACCTGACATGCGAC | PEEDDITWTSDQRHGV | |
| | ACCCCCGAGGAGGACGATATCACCTGGACATCTGATCAGAGGCAC | IGSGKTLTITVKEFLD | |
| | GGCGTGATCGGAAGCGGCAAGACCCTGACAATCACCGTGAAGGAG | AGQYTCHKGGETLSHS | |
| | TTCCTGGATGCCGGCCAGTACACATGTCACAAGGGCGGCGAGACC | HLLLHKKENGIWSTEI | |
| | CTGTCCCACTCTCACCTGCTGCTGCACAAGAAGGAGAACGGCATC | LKNFKNKTFLKCEAPN | |
| | TGGTCCACAGAGATCCTGAAGAACTTCAAGAATAAGACCTTTCTG | YSGRFTCSWLVQRNMD | |
| | AAGTGCGAGGCCCCTAATTATAGCGGCCGGTTCACCTGTTCCTGG | LKFNIKSSSSSPDSRA | |
| | CTGGTGCAGAGAAACATGGACCTGAAGTTTAATATCAAGAGCTCC | VTCGMASLSAEKVTLD | |
| | TCTAGCTCCCCAGATAGCCGGGCAGTGACATGCGGAATGGCCAGC | QRDYEKYSVSCQEDVT | |
| | CTGTCCGCCGAGAAGGTGACCCTGGACCAGAGAGATTACGAGAAG | CPTAEETLPIELALEA | |
| | TATTCTGTGAGCTGCCAGGAGGACGTGACATGTCCCACCGCCGAG | RQQNKYENYSTSFFIR | |
| | GAGACACTGCCTATCGAGCTGGCCCTGGAGGCCAGGCAGCAGAAC | DIIKPDPPKNLQMKPL | |
| | AAGTACGAGAATTATTCCACCTCTTTCTTTATCCGCGACATCATC | KNSQVEVSWEYPDSWS | |
| | AAGCCAGATCCCCCTAAGAACCTGCAGATGAAGCCCCTGAAGAAT | TPHSYFSLKFFVRIQR | |
| | TCCCAGGTCGAGGTGTCTTGGGAGTACCCTGACAGCTGGTCCACA | KKEKMKETEEGCNQKG | |
| | CCACACTCTTATTTCAGCCTGAAGTTCTTTGTGAGGATCCAGCGC | AFLVEKTSTEVQCKGG | |
| | AAGAAGGAGAAGATGAAGGAGACCGAGGAGGGCTGCAATCAGAAG | NVCVQAQDRYYNSSCS | |
| | GGCGCCTTTCTGGTGGAGAAGACATCCACCGAGGTGCAGTGCAAG | KWACVPCRVRSGTAEA | |
| | GGAGGAAACGTGTGCGTGCAGGCACAGGATCGGTACTATAATTCT | ASASGEAGRSANHTPA | |
| | AGCTGTTCCAAGTGGGCCTGCGTGCCTTGTCGGGTGAGATCTGGC | GLTGPGTSESATPESG | |
| | ACAGCCGAGGCCGCTAGCGCCAGCGGCGAGGCCGGCCGGAGCGCC | PGSEPATSGSETPGTS | |
| | AACCACACCCCCGCCGGCCTGACCGGCCCTGGTACCTCAGAGTCT | ESATPESGPGSEPATS | |
| | GCTACCCCCGAGTCAGGGCCAGGATCAGAGCCAGCCACCTCCGGG | GSETPGTSESATPESG | |
| | TCTGAGACACCCGGGACTTCCGAGAGTGCCACCCCTGAGTCCGGA | PGTSTEPSEGSAPGSP | |
| | CCCGGGTCCGAGCCCGCCACTTCCGGCTCCGAAACTCCCGGCACA | AGSPTSTEEGTSESAT | |
| | AGCGAGAGCGCTACCCCAGAGTCAGGACCAGGAACATCTACAGAG | PESGPGSEPATSGSET | |
| | CCCTCTGAAGGCTCCGCTCCAGGGTCCCCAGCCGGCAGTCCCACT | PGTSESATPESGPGSP | |
| | AGCACCGAGGAGGGAACCTCTGAAAGCGCCACACCCGAATCAGGG | AGSPTSTEEGSPAGSP | |
| | CCAGGGTCTGAGCCTGCTACCAGCGGCAGCGAGACACCAGGCACC | TSTEEGTSTEPSEGSA | |
| | TCTGAGTCCGCCACACCAGAGTCCGGACCCGGATCTCCCGCTGGG | PGTSESATPESGPGTS | |
| | AGCCCCACCTCCACTGAGGAGGGATCTCCTGCTGGCTCTCCAACA | ESATPESGPGTSESAT | |
| | TCTACTGAGGAAGGTACCTCAACCGAGCCATCCGAGGGATCAGCT | PESGPGSEPATSGSET | |
| | CCCGGCACCTCAGAGTCGGCAACCCCGGAGTCTGGACCCGGAACT | PGSEPATSGSETPGSP | |
| | TCCGAAAGTGCCACACCAGAGTCCGGTCCCGGGACTTCAGAATCA | AGSPTSTEEGTSTEPS | |
| | GCAACACCCGAGTCCGGCCCTGGGTCTGAACCCGCCACAAGTGGT | EGSAPGTSTEPSEGSA | |
| | AGTGAGACACCAGGATCAGAACCTGCTACCTCAGGGTCAGAGACA | PGSEPATSGSETPGTS | |
| | CCCGGATCTCCGGCAGGCTCACCAACCTCCACTGAGGAGGGCACC | ESATPESGPGTSTEPS | |
| | AGCACAGAACCAAGCGAGGGCTCCGCACCCGGAACAAGCACTGAA | EGSAP (SEQ ID | |
| | CCCAGTGAGGGTTCAGCACCCGGCTCTGAGCCGGCCACAAGTGGC | NO: 851) | |
| | AGTGAGACACCCGGCACTTCAGAGAGTGCCACCCCCGAGAGTGGC | | |
| | CCAGGCACTAGTACCGAGCCCTCTGAAGGCAGTGCGCCACAT | | |
| | (SEQ ID NO: 833) | | |
| XP04 | GGTTCTCCAGCCGGGTCCCCAACTTCGACCGAGGAAGGGACCTCC | GSPAGSPTSTEEGTSE | P40 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | GSPAGSPTSTEEGTSE | |
| | AGCGGGCCAGGTTCTCCTGCTGGCTCCCCCACCTCAACAGAAGAG | SATPESGPGSEPATSG | |
| | GGGACAAGCGAAAGCGCTACGCCTGAGAGTGGCCCTGGCTCTGAG | SETPGTSESATPESGP | |
| | CCAGCCACCTCCGGCTCTGAAACCCCTGGCACTAGTGAGTCTGCC | GTSTEPSEGSAPGTST | |
| | ACGCCTGAGTCCGGACCCGGGACCTCTACTGAGCCCTCGGAGGGG | EPSEGSAPGTSTEPSE | |
| | AGCGCTCCTGGCACGAGTACAGAACCTTCCGAAGGAAGTGCACCG | GSAPGTSTEAGRSANH | |
| | GGCACAAGCACCGAGCCTTCCGAAGGCTCTGCTCCCGGAACCTCT | TPAGLTGPGTAEAASA | |
| | ACCGAGGCCGGCCGGAGCGCCAACCACACCCCCGCCGGCCTGACC | SGMWELEKDVYVVEVD | |
| | GGCCCTGGCACAGCCGAGGCCGCTAGCGCCAGCGGCATGTGGGAG | WTPDAPGETVNLTCDT | |
| | CTGGAGAAGGACGTGTACGTGGTGGAGGTGGACTGGACACCAGAT | PEEDDITWTSDQRHGV | |
| | GCCCCCGGCGAGACCGTGAACCTGACATGCGACACCCCCGAGGAG | IGSGKTLTITVKEFLD | |
| | GACGATATCACCTGGACATCTGATCAGAGGCACGGCGTGATCGGA | AGQYTCHKGGETLSHS | |
| | AGCGGCAAGACCCTGACAATCACCGTGAAGGAGTTCCTGGATGCC | HLLLHKKENGIWSTEI | |
| | GGCCAGTACACATGTCACAAGGGCGGCGAGACCCTGTCCCACTCT | LKNFKNKTFLKCEAPN | |
| | CACCTGCTGCTGCACAAGAAGGAGAACGGCATCTGGTCCACAGAG | YSGRFTCSWLVQRNMD | |
| | ATCCTGAAGAACTTCAAGAATAAGACCTTTCTGAAGTGCGAGGCC | LKFNIKSSSSSPDSRA | |
| | CCTAATTATAGCGGCCGGTTCACCTGTTCCTGGCTGGTGCAGAGA | VTCGMASLSAEKVTLD | |
| | AACATGGACCTGAAGTTTAATATCAAGAGCTCCTCTAGCTCCCCA | QRDYEKYSVSCQEDVT | |
| | GATAGCCGGGCAGTGACATGCGGAATGGCCAGCCTGTCCGCCGAG | CPTAEETLPIELALEA | |
| | AAGGTGACCCTGGACCAGAGAGATTACGAGAAGTATTCTGTGAGC | RQQNKYENYSTSFFIR | |
| | TGCCAGGAGGACGTGACATGTCCCACCGCCGAGGAGACACTGCCT | DIIKPDPPKNLQMKPL | |
| | ATCGAGCTGGCCCTGGAGGCCAGGCAGCAGAACAAGTACGAGAAT | KNSQVEVSWEYPDSWS | |
| | TATTCCACCTCTTTCTTTATCCGCGACATCATCAAGCCAGATCCC | TPHSYFSLKFFVRIQR | |
| | CCTAAGAACCTGCAGATGAAGCCCCTGAAGAATTCCCAGGTCGAG | KKEKMKETEEGCNQKG | |
| | GTGTCTTGGGAGTACCCTGACAGCTGGTCCACACCACACTCTTAT | AFLVEKTSTEVQCKGG | |
| | TTCAGCCTGAAGTTCTTTGTGAGGATCCAGCGCAAGAAGGAGAAG | NVCVQAQDRYYNSSCS | |
| | ATGAAGGAGACCGAGGAGGGCTGCAATCAGAAGGGCGCCTTTCTG | KWACVPCRVRSGTAEA | |
| | GTGGAGAAGACATCCACCGAGGTGCAGTGCAAGGGAGGAAACGTG | ASASGEAGRSANHTPA | |
| | TGCGTGCAGGCACAGGATCGGTACTATAATTCTAGCTGTTCCAAG | GLTGPGTSESATPESG | |
| | TGGGCCTGCGTGCCTTGTCGGGTGAGATCTGGCACAGCCGAGGCC | PGSEPATSGSETPGTS | |
| | GCTAGCGCCAGCGGCGAGGCCGGCCGGAGCGCCAACCACACCCCC | ESATPESGPGSEPATS | |
| | GCCGGCCTGACCGGCCCTGGTACCTCAGAGTCTGCTACCCCCGAG | GSETPGTSESATPESG | |
| | TCAGGGCCAGGATCAGAGCCAGCCACCTCCGGGTCTGAGACACCC | PGTSTEPSEGSAPGSP | |
| | GGGACTTCCGAGAGTGCCACCCCTGAGTCCGGACCCGGGTCCGAG | AGSPTSTEEGTSESAT | |
| | CCCGCCACTTCCGGCTCCGAAACTCCCGGCACAAGCGAGAGCGCT | PESGPGSEPATSGSET | |
| | ACCCCAGAGTCAGGACCAGGAACATCTACAGAGCCCTCTGAAGGC | PGTSESATPESGPGSP | |
| | TCCGCTCCAGGGTCCCCAGCCGGCAGTCCCACTAGCACCGAGGAG | AGSPTSTEEGSPAGSP | |
| | GGAACCTCTGAAAGCGCCACACCCGAATCAGGGCCAGGGTCTGAG | TSTEEGTSTEPSEGSA | |
| | CCTGCTACCAGCGGCAGCGAGACACCAGGCACCTCTGAGTCCGCC | PGTSESATPESGPGTS | |
| | ACACCAGAGTCCGGACCCGGATCTCCCGCTGGGAGCCCCACCTCC | ESATPESGPGTSESAT | |
| | ACTGAGGAGGGATCTCCTGCTGGCTCTCCAACATCTACTGAGGAA | PESGPGSEPATSGSET | |
| | GGTACCTCAACCGAGCCATCCGAGGGATCAGCTCCCGGCACCTCA | PGSEPATSGSETPGSP | |
| | GAGTCGGCAACCCCGGAGTCTGGACCCGGAACTTCCGAAAGTGCC | AGSPTSTEEGTSTEPS | |
| | ACACCAGAGTCCGGTCCCGGGACTTCAGAATCAGCAACACCCGAG | EGSAPGTSTEPSEGSA | |
| | TCCGGCCCTGGGTCTGAACCCGCCACAAGTGGTAGTGAGACACCA | PGSEPATSGSETPGTS | |
| | GGATCAGAACCTGCTACCTCAGGGTCAGAGACACCCGGATCTCCG | ESATPESGPGTSTEPS | |
| | GCAGGCTCACCAACCTCCACTGAGGAGGGCACCAGCACAGAACCA | EGSAP (SEQ ID | |
| | AGCGAGGGCTCCGCACCCGGAACAAGCACTGAACCCAGTGAGGGT | NO: 852) | |
| | TCAGCACCCGGCTCTGAGCCGGCCACAAGTGGCAGTGAGACACCC | | |
| | GGCACTTCAGAGAGTGCCACCCCCGAGAGTGGCCCAGGCACTAGT | | |
| | ACCGAGCCCTCTGAAGGCAGTGCGCCA (SEQ ID NO: 834) | | |
| XP05 | GGTTCTCCAGCCGGGTCCCCAACTTCGACCGAGGAAGGGACCTCC | GSPAGSPTSTEEGTSE | P40 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGMWELEK | |
| | CTGACCGGCCCTGGCACAGCCGAGGCCGCTAGCGCCAGCGGCATG | DVYVVEVDWTPDAPGE | |
| | TGGGAGCTGGAGAAGGACGTGTACGTGGTGGAGGTGGACTGGACA | TVNLTCDTPEEDDITW | |
| | CCAGATGCCCCCGGCGAGACCGTGAACCTGACATGCGACACCCCC | TSDQRHGVIGSGKTLT | |
| | GAGGAGGACGATATCACCTGGACATCTGATCAGAGGCACGGCGTG | ITVKEFLDAGQYTCHK | |
| | ATCGGAAGCGGCAAGACCCTGACAATCACCGTGAAGGAGTTCCTG | GGETLSHSHLLLHKKE | |
| | GATGCCGGCCAGTACACATGTCACAAGGGCGGCGAGACCCTGTCC | NGIWSTEILKNFKNKT | |
| | CACTCTCACCTGCTGCTGCACAAGAAGGAGAACGGCATCTGGTCC | FLKCEAPNYSGRFTCS | |
| | ACAGAGATCCTGAAGAACTTCAAGAATAAGACCTTTCTGAAGTGC | WLVQRNMDLKFNIKSS | |
| | GAGGCCCCTAATTATAGCGGCCGGTTCACCTGTTCCTGGCTGGTG | SSSPDSRAVTCGMASL | |
| | CAGAGAAACATGGACCTGAAGTTTAATATCAAGAGCTCCTCTAGC | SAEKVTLDQRDYEKYS | |
| | TCCCCAGATAGCCGGGCAGTGACATGCGGAATGGCCAGCCTGTCC | VSCQEDVTCPTAEETL | |
| | GCCGAGAAGGTGACCCTGGACCAGAGAGATTACGAGAAGTATTCT | PIELALEARQQNKYEN | |
| | GTGAGCTGCCAGGAGGACGTGACATGTCCCACCGCCGAGGAGACA | YSTSFFIRDIIKPDPP | |
| | CTGCCTATCGAGCTGGCCCTGGAGGCCAGGCAGCAGAACAAGTAC | KNLQMKPLKNSQVEVS | |
| | GAGAATTATTCCACCTCTTTCTTTATCCGCGACATCATCAAGCCA | WEYPDSWSTPHSYFSL | |
| | GATCCCCCTAAGAACCTGCAGATGAAGCCCCTGAAGAATTCCCAG | KFFVRIQRKKEKMKET | |
| | GTCGAGGTGTCTTGGGAGTACCCTGACAGCTGGTCCACACCACAC | EEGCNQKGAFLVEKTS | |
| | TCTTATTTCAGCCTGAAGTTCTTTGTGAGGATCCAGCGCAAGAAG | TEVQCKGGNVCVQAQD | |
| | GAGAAGATGAAGGAGACCGAGGAGGGCTGCAATCAGAAGGGCGCC | RYYNSSCSKWACVPCR | |
| | TTTCTGGTGGAGAAGACATCCACCGAGGTGCAGTGCAAGGGAGGA | VRSGTAEAASASGEAG | |
| | AACGTGTGCGTGCAGGCACAGGATCGGTACTATAATTCTAGCTGT | RSANHTPAGLTGPGTS | |
| | TCCAAGTGGGCCTGCGTGCCTTGTCGGGTGAGATCTGGCACAGCC | ESATPESGPGSEPATS | |
| | GAGGCCGCTAGCGCCAGCGGCGAGGCCGGCCGGAGCGCCAACCAC | GSETPGTSESATPESG | |
| | ACCCCCGCCGGCCTGACCGGCCCTGGTACCTCAGAGTCTGCTACC | PGSEPATSGSETPGTS | |
| | CCCGAGTCAGGGCCAGGATCAGAGCCAGCCACCTCCGGGTCTGAG | ESATPESGPGTSTEPS | |
| | ACACCCGGGACTTCCGAGAGTGCCACCCCTGAGTCCGGACCCGGG | EGSAPGSPAGSPTSTE | |
| | TCCGAGCCCGCCACTTCCGGCTCCGAAACTCCCGGCACAAGCGAG | EGTSESATPESGPGSE | |
| | AGCGCTACCCCAGAGTCAGGACCAGGAACATCTACAGAGCCCTCT | PATSGSETPGTSESAT | |
| | GAAGGCTCCGCTCCAGGGTCCCCAGCCGGCAGTCCCACTAGCACC | PESGPGSPAGSPTSTE | |
| | GAGGAGGGAACCTCTGAAAGCGCCACACCCGAATCAGGGCCAGGG | EGSPAGSPTSTEEGTS | |
| | TCTGAGCCTGCTACCAGCGGCAGCGAGACACCAGGCACCTCTGAG | TEPSEGSAPGTSESAT | |
| | TCCGCCACACCAGAGTCCGGACCCGGATCTCCCGCTGGGAGCCCC | PESGPGTSESATPESG | |
| | ACCTCCACTGAGGAGGGATCTCCTGCTGGCTCTCCAACATCTACT | PGTSESATPESGPGSE | |
| | GAGGAAGGTACCTCAACCGAGCCATCCGAGGGATCAGCTCCCGGC | PATSGSETPGSEPATS | |
| | ACCTCAGAGTCGGCAACCCCGGAGTCTGGACCCGGAACTTCCGAA | GSETPGSPAGSPTSTE | |
| | AGTGCCACACCAGAGTCCGGTCCCGGGACTTCAGAATCAGCAACA | EGTSTEPSEGSAPGTS | |
| | CCCGAGTCCGGCCCTGGGTCTGAACCCGCCACAAGTGGTAGTGAG | TEPSEGSAPGSEPATS | |
| | ACACCAGGATCAGAACCTGCTACCTCAGGGTCAGAGACACCCGGA | GSETPGTSESATPESG | |
| | TCTCCGGCAGGCTCACCAACCTCCACTGAGGAGGGCACCAGCACA | PGTSTEPSEGSAP | |
| | GAACCAAGCGAGGGCTCCGCACCCGGAACAAGCACTGAACCCAGT | (SEQ ID NO: 853) | |
| | GAGGGTTCAGCACCCGGCTCTGAGCCGGCCACAAGTGGCAGTGAG | | |
| | ACACCCGGCACTTCAGAGAGTGCCACCCCCGAGAGTGGCCCAGGC | | |
| | ACTAGTACCGAGCCCTCTGAAGGCAGTGCGCCA (SEQ ID NO: | | |
| | 835) | | |
| XP06 | GGTTCTCCAGCCGGGTCCCCAACTTCGACCGAGGAAGGGACCTCC | GSPAGSPTSTEEGTSE | P40 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | GSPAGSPTSTEEGTSE | |
| | AGCGGGCCAGGTTCTCCTGCTGGCTCCCCCACCTCAACAGAAGAG | SATPESGPGSEPATSG | |
| | GGGACAAGCGAAAGCGCTACGCCTGAGAGTGGCCCTGGCTCTGAG | SETPGTSESATPESGP | |
| | CCAGCCACCTCCGGCTCTGAAACCCCTGGCACTAGTGAGTCTGCC | GTSTEPSEGSAPGTST | |
| | ACGCCTGAGTCCGGACCCGGGACCTCTACTGAGCCCTCGGAGGGG | EPSEGSAPGTSTEPSE | |
| | AGCGCTCCTGGCACGAGTACAGAACCTTCCGAAGGAAGTGCACCG | GSAPGTSTEPSEGSAP | |
| | GGCACAAGCACCGAGCCTTCCGAAGGCTCTGCTCCCGGAACCTCT | GTSTEPSEGSAPGTST | |
| | ACCGAACCCTCTGAAGGGTCTGCACCCGGCACGAGCACCGAACCC | EPSEGSAPGSPAGSPT | |
| | AGCGAAGGGTCAGCGCCTGGGACCTCAACAGAGCCCTCGGAAGGA | STEEGTSTEPSEGSAP | |
| | TCAGCGCCTGGAAGCCCTGCAGGGAGTCCAACTTCCACGGAAGAA | EAGRSANHTPAGLTGP | |
| | GGAACGTCTACAGAGCCATCAGAGGGGTCCGCACCAGAGGCCGGC | GTAEAASASGMWELEK | |
| | CGGAGCGCCAACCACACCCCCGCCGGCCTGACCGGCCCTGGCACA | DVYVVEVDWTPDAPGE | |
| | GCCGAGGCCGCTAGCGCCAGCGGCATGTGGGAGCTGGAGAAGGAC | TVNLTCDTPEEDDITW | |
| | GTGTACGTGGTGGAGGTGGACTGGACACCAGATGCCCCCGGCGAG | TSDQRHGVIGSGKTLT | |
| | ACCGTGAACCTGACATGCGACACCCCCGAGGAGGACGATATCACC | ITVKEFLDAGQYTCHK | |
| | TGGACATCTGATCAGAGGCACGGCGTGATCGGAAGCGGCAAGACC | GGETLSHSHLLLHKKE | |
| | CTGACAATCACCGTGAAGGAGTTCCTGGATGCCGGCCAGTACACA | NGIWSTEILKNFKNKT | |
| | TGTCACAAGGGCGGCGAGACCCTGTCCCACTCTCACCTGCTGCTG | FLKCEAPNYSGRFTCS | |
| | CACAAGAAGGAGAACGGCATCTGGTCCACAGAGATCCTGAAGAAC | WLVQRNMDLKFNIKSS | |
| | TTCAAGAATAAGACCTTTCTGAAGTGCGAGGCCCCTAATTATAGC | SSSPDSRAVTCGMASL | |
| | GGCCGGTTCACCTGTTCCTGGCTGGTGCAGAGAAACATGGACCTG | SAEKVTLDQRDYEKYS | |
| | AAGTTTAATATCAAGAGCTCCTCTAGCTCCCCAGATAGCCGGGCA | VSCQEDVTCPTAEETL | |
| | GTGACATGCGGAATGGCCAGCCTGTCCGCCGAGAAGGTGACCCTG | PIELALEARQQNKYEN | |
| | GACCAGAGAGATTACGAGAAGTATTCTGTGAGCTGCCAGGAGGAC | YSTSFFIRDIIKPDPP | |
| | GTGACATGTCCCACCGCCGAGGAGACACTGCCTATCGAGCTGGCC | KNLQMKPLKNSQVEVS | |
| | CTGGAGGCCAGGCAGCAGAACAAGTACGAGAATTATTCCACCTCT | WEYPDSWSTPHSYFSL | |
| | TTCTTTATCCGCGACATCATCAAGCCAGATCCCCCTAAGAACCTG | KFFVRIQRKKEKMKET | |
| | CAGATGAAGCCCCTGAAGAATTCCCAGGTCGAGGTGTCTTGGGAG | EEGCNQKGAFLVEKTS | |
| | TACCCTGACAGCTGGTCCACACCACACTCTTATTTCAGCCTGAAG | TEVQCKGGNVCVQAQD | |
| | TTCTTTGTGAGGATCCAGCGCAAGAAGGAGAAGATGAAGGAGACC | RYYNSSCSKWACVPCR | |
| | GAGGAGGGCTGCAATCAGAAGGGCGCCTTTCTGGTGGAGAAGACA | VRSGTAEAASASGEAG | |
| | TCCACCGAGGTGCAGTGCAAGGGAGGAAACGTGTGCGTGCAGGCA | RSANHTPAGLTGPGTS | |
| | CAGGATCGGTACTATAATTCTAGCTGTTCCAAGTGGGCCTGCGTG | ESATPESGPGSEPATS | |
| | CCTTGTCGGGTGAGATCTGGCACAGCCGAGGCCGCTAGCGCCAGC | GSETPGTSESATPESG | |
| | GGCGAGGCCGGCCGGAGCGCCAACCACACCCCCGCCGGCCTGACC | PGSEPATSGSETPGTS | |
| | GGCCCTGGTACCAGCGAATCCGCTACTCCCGAATCTGGCCCTGGG | ESATPESGPGTSTEPS | |
| | TCCGAACCTGCCACCTCCGGCTCTGAAACTCCAGGGACCTCCGAA | EGSAPGTSESATPESG | |
| | TCTGCCACACCCGAGAGCGGCCCTGGCTCCGAGCCCGCAACATCT | PGSPAGSPTSTEEGSP | |
| | GGCAGCGAGACACCTGGCACCTCCGAGAGCGCAACACCCGAGAGC | AGSPTSTEEGSPAGSP | |
| | GGCCCTGGCACCAGCACCGAGCCATCCGAGGGATCCGCCCCAGGC | TSTEEGTSESATPESG | |
| | ACTTCTGAGTCAGCCACACCCGAAAGCGGACCAGGATCACCCGCT | PGTSTEPSEGSAPGTS | |
| | GGCTCCCCCACCAGTACCGAGGAGGGGTCCCCCGCTGGAAGTCCA | ESATPESGPGSEPATS | |
| | ACAAGCACTGAGGAAGGGTCCCCTGCCGGCTCCCCCACAAGTACC | GSETPGTSESATPESG | |
| | GAAGAGGGCACAAGTGAGAGCGCCACTCCCGAGTCCGGGCCTGGC | PGSEPATSGSETPGTS | |
| | ACCAGCACAGAGCCTTCCGAGGGGTCCGCACCAGGTACCTCAGAG | ESATPESGPGTSTEPS | |
| | TCTGCTACCCCCGAGTCAGGGCCAGGATCAGAGCCAGCCACCTCC | EGSAPGSPAGSPTSTE | |
| | GGGTCTGAGACACCCGGGACTTCCGAGAGTGCCACCCCTGAGTCC | EGTSESATPESGPGSE | |
| | GGACCCGGGTCCGAGCCCGCCACTTCCGGCTCCGAAACTCCCGGC | PATSGSETPGTSESAT | |
| | ACAAGCGAGAGCGCTACCCCAGAGTCAGGACCAGGAACATCTACA | PESGPGSPAGSPTSTE | |
| | GAGCCCTCTGAAGGCTCCGCTCCAGGGTCCCCAGCCGGCAGTCCC | EGSPAGSPTSTEEGTS | |
| | ACTAGCACCGAGGAGGGAACCTCTGAAAGCGCCACACCCGAATCA | TEPSEGSAPGTSESAT | |
| | GGGCCAGGGTCTGAGCCTGCTACCAGCGGCAGCGAGACACCAGGC | PESGPGTSESATPESG | |
| | ACCTCTGAGTCCGCCACACCAGAGTCCGGACCCGGATCTCCCGCT | PGTSESATPESGPGSE | |
| | GGGAGCCCCACCTCCACTGAGGAGGGATCTCCTGCTGGCTCTCCA | PATSGSETPGSEPATS | |
| | ACATCTACTGAGGAAGGTACCTCAACCGAGCCATCCGAGGGATCA | GSETPGSPAGSPTSTE | |
| | GCTCCCGGCACCTCAGAGTCGGCAACCCCGGAGTCTGGACCCGGA | EGTSTEPSEGSAPGTS | |
| | ACTTCCGAAAGTGCCACACCAGAGTCCGGTCCCGGGACTTCAGAA | TEPSEGSAPGSEPATS | |
| | TCAGCAACACCCGAGTCCGGCCCTGGGTCTGAACCCGCCACAAGT | GSETPGTSESATPESG | |
| | GGTAGTGAGACACCAGGATCAGAACCTGCTACCTCAGGGTCAGAG | PGTSTEPSEGSAP | |
| | ACACCCGGATCTCCGGCAGGCTCACCAACCTCCACTGAGGAGGGC | (SEQ ID NO: 854) | |
| | ACCAGCACAGAACCAAGCGAGGGCTCCGCACCCGGAACAAGCACT | | |
| | GAACCCAGTGAGGGTTCAGCACCCGGCTCTGAGCCGGCCACAAGT | | |
| | GGCAGTGAGACACCCGGCACTTCAGAGAGTGCCACCCCCGAGAGT | | |
| | GGCCCAGGCACTAGTACCGAGCCCTCTGAAGGCAGTGCGCCA | | |
| | (SEQ ID NO: 836) | | |
| XP07 | GCAAGCTCCGCCACCCCCGAGTCTGGACCAGGCACCAGCACAGAG | ASSATPESGPGTSTEP | P40 |
| | CCTTCTGAGGGAAGCGCCCCAGGCACAAGCGAGTCCGCCACCCCT | SEGSAPGTSESATPES | |
| | GAGTCCGGACCAGGATCTGGACCAGCCACCTCTGAGAGCGCCACA | GPGSGPATSESATPGT | |
| | CCTGGCACCTCCGAGTCTGCCACACCTGAGAGCGGACCAGGATCC | SESATPESGPGSEPAT | |
| | GAGCCAGCCACCAGCGGCTCCGAGACACCAGGCACCTCTGAAAGC | SGSETPGTSESATPES | |
| | GCCACTCCTGAGTCCGGACCAGGCACCTCTACAGAGCCTTCCGAG | GPGTSTEPSEGSAPGS | |
| | GGATCTGCCCCAGGAAGCCCAGCAGGCAGCCCAACCTCCACAGAG | PAGSPTSTEEGTSESA | |
| | GAGGGCACATCCGAGTCTGCCACTCCTGAGTCTGGACCTGGAAGC | TPESGPGSEPATSGSE | |
| | GAGCCAGCCACAAGCGGAAGCGAAACACCAGGCACCTCTGAGAGC | TPGTSESATPESGPGS | |
| | GCCACGCCTGAGTCCGGACCTGGATCTCCAGCCGGCTCTCCTACC | PAGSPTSTEEGSPAGS | |
| | AGCACAGAGGAGGGATCCCCAGCAGGATCCCCTACCTCTACAGAG | PTSTEEGTSTEPSEGS | |
| | GAGGGCACCAGCACAGAGCCAAGCGAGGGATCCGCCCCTGGCACA | APGTSESATPESGPGT | |
| | TCCGAATCTGCCACCCCAGAGTCCGGACCTGGCACAAGCGAATCC | SESATPESGPGTSESA | |
| | GCCACCCCTGAGAGCGGACCAGGCACATCTGAGAGCGCCACCCCA | TPESGPGSEPATSGSE | |
| | GAGAGCGGACCTGGATCCGAGCCAGCCACATCCGGATCTGAGACC | TPGSEPATSGSETPGS | |
| | CCAGGATCCGAGCCTGCCACAAGCGGATCCGAGACCCCAGGAAGC | PAGSPTSTEEGTSTEP | |
| | CCTGCAGGATCTCCCACCAGCACCGAAGAAGGCACCAGCACCGAG | SEGSAPGTSTEPSEGS | |
| | CCCAGCGAAGGATCTGCCCCTGGCACCAGCACCGAGCCTAGCGAG | APGSEPATSGSETPGT | |
| | GGATCCGCCCCCGGCTCCGAGCCAGCCACCTCTGGAAGTGAAACA | SESATPEAGRSANHTP | |
| | CCAGGCACCTCCGAATCTGCCACACCAGAGGCAGGCCGGTCCGCC | AGLTGPGTSESATPES | |
| | AACCACACCCCAGCAGGACTGACAGGACCAGGCACCAGCGAATCC | MWELEKDVYVVEVDWT | |
| | GCCACTCCAGAGAGCATGTGGGAGCTGGAGAAGGACGTGTACGTG | PDAPGETVNLTCDTPE | |
| | GTGGAGGTGGACTGGACACCAGATGCCCCCGGCGAGACCGTGAAT | EDDITWTSDQRHGVIG | |
| | CTGACATGCGACACCCCCGAGGAGGACGATATCACCTGGACATCC | SGKTLTITVKEFLDAG | |
| | GATCAGAGACACGGCGTGATCGGCTCTGGCAAGACCCTGACAATC | QYTCHKGGETLSHSHL | |
| | ACCGTGAAGGAGTTCCTGGATGCCGGCCAGTACACATGTCACAAG | LLHKKENGIWSTEILK | |
| | GGCGGCGAGACCCTGTCTCACAGCCACCTGCTGCTGCACAAGAAG | NFKNKTFLKCEAPNYS | |
| | GAGAACGGCATCTGGTCCACAGAGATCCTGAAGAACTTCAAGAAT | GRFTCSWLVQRNMDLK | |
| | AAGACCTTTCTGAAGTGCGAGGCCCCAAATTATAGCGGCCGGTTC | FNIKSSSSSPDSRAVT | |
| | ACCTGTTCCTGGCTGGTGCAGAGAAACATGGACCTGAAGTTTAAT | CGMASLSAEKVTLDQR | |
| | ATCAAGTCTAGCTCCTCTAGCCCAGATAGCAGGGCAGTGACATGC | DYEKYSVSCQEDVTCP | |
| | GGAATGGCATCCCTGTCTGCCGAGAAGGTGACCCTGGACCAGAGA | TAEETLPIELALEARQ | |
| | GATTACGAGAAGTATAGCGTGTCCTGCCAGGAGGACGTGACATGT | QNKYENYSTSFFIRDI | |
| | CCTACCGCCGAGGAGACCCTGCCAATCGAGCTGGCCCTGGAGGCC | IKPDPPKNLQMKPLKN | |
| | AGGCAGCAGAACAAGTACGAGAATTATTCTACCAGCTTCTTTATC | SQVEVSWEYPDSWSTP | |
| | CGCGACATCATCAAGCCAGATCCCCCTAAGAACCTGCAGATGAAG | HSYFSLKFFVRIQRKK | |
| | CCCCTGAAGAATAGCCAGGTCGAGGTGTCCTGGGAGTACCCTGAC | EKMKETEEGCNQKGAF | |
| | TCCTGGTCTACCCCACACTCTTATTTCAGCCTGAAGTTCTTTGTG | LVEKTSTEVQCKGGNV | |
| | AGGATCCAGCGCAAGAAGGAGAAGATGAAGGAGACCGAGGAGGGC | CVQAQDRYYNSSCSKW | |
| | TGCAACCAGAAGGGCGCCTTTCTGGTGGAGAAGACATCCACCGAG | ACVPCRVRSGTATPES | |
| | GTGCAGTGCAAGGGAGGAAACGTGTGCGTGCAGGCACAGGATAGG | GPGEAGRSANHTPAGL | |
| | TACTATAATTCCTCTTGTAGCAAGTGGGCCTGCGTGCCCTGTCGG | TGPATPESGPGSPAGS | |
| | GTGAGATCTGGCACAGCTACTCCAGAAAGCGGACCAGGAGAGGCA | PTSTEEGSPAGSPTST | |
| | GGCCGCAGCGCCAATCATACTCCTGCCGGACTGACAGGACCTGCA | EEGSPAGSPTSTEEGT | |
| | ACTCCTGAGTCTGGACCCGGCAGCCCTGCAGGATCCCCCACATCT | SESATPESGPGTSTEP | |
| | ACCGAAGAAGGATCCCCAGCAGGAAGCCCTACATCCACCGAGGAG | SEGSAPGTSESATPES | |
| | GGAAGCCCAGCAGGATCTCCCACAAGCACCGAGGAGGGCACAAGC | GPGSEPATSGSETPGT | |
| | GAGTCCGCCACGCCTGAGTCTGGACCAGGCACAAGCACCGAGCCA | SESATPESGPGSEPAT | |
| | TCCGAGGGATCTGCCCCTGGCACATCTGAAAGCGCCACTCCCGAA | SGSETPGTSESATPES | |
| | AGCGGACCTGGATCTGAGCCAGCCACCTCCGGATCTGAGACACCA | GPGTSTEPSEGSAPGS | |
| | GGCACCAGCGAGTCCGCCACACCCGAATCCGGCCCAGGCAGCGAA | PAGSPTSTEEGTSESA | |
| | CCTGCCACCTCTGGAAGCGAGACCCCAGGCACCTCCGAGTCTGCC | TPESGPGSEPATSGSE | |
| | ACGCCCGAATCCGGACCTGGCACATCTACCGAACCTTCCGAAGGA | TPGTSESATPESGPGS | |
| | TCCGCCCCTGGCAGCCCAGCAGGATCTCCTACAAGCACTGAAGAG | PAGSPTSTEEGSPAGS | |
| | GGCACAAGCGAGTCCGCCACTCCAGAGTCTGGACCAGGAAGCGAG | PTSTEEGTSTEPSEGS | |
| | CCTGCCACCTCTGGCAGCGAGACCCCCGGCACCTCCGAGTCTGCC | APGTSESATPESGPGT | |
| | ACCCCTGAATCTGGCCCTGGATCTCCAGCCGGGTCCCCCACATCT | SESATPESGPGTSPSA | |
| | ACCGAGGAAGGCTCCCCAGCAGGAAGCCCCACATCCACTGAAGAA | TPESGPGSEPATSGSE | |
| | GGCACAAGCACTGAACCATCCGAAGGCAGCGCCCCTGGCACAAGC | TPGSEPATSGSETPGS | |
| | GAGTCCGCCACACCAGAGTCAGGCCCTGGCACATCTGAGAGCGCC | PAGSPTSTEEGTSTEP | |
| | ACGCCAGAGAGCGGACCTGGCACATCCCCATCTGCCACTCCTGAG | SEGSAPGTSTEPSEGS | |
| | AGTGGCCCCGGGTCTGAACCAGCCACAAGCGGCAGCGAAACTCCT | APGSEPATSGSETPGT | |
| | GGCTCCGAGCCTGCCACATCTGGGTCCGAAACTCCTGGCTCCCCA | SESAGEPEA (SEQ | |
| | GCCGGCAGCCCCACATCTACTGAGGAGGGCACAAGCACTGAACCC | ID NO: 855) | |
| | TCCGAGGGATCCGCCCCAGGCACATCTACCGAGCCCTCCGAAGGA | | |
| | AGCGCCCCAGGAAGCGAACCTGCCACCTCCGGCTCCGAAACCCCT | | |
| | GGCACCAGCGAATCCGCCGGAGAGCCTGAGGCC (SEQ ID NO: | | |
| | 837) | | |
| XP08 | GGTTCTCCAGCCGGGTCCCCAACTTCGACCGAGGAAGGGACCTCC | EPEAGTAEAASASGGT | P35 |
| | GAGTCAGCTACCCCGGAGTCCGGTCCTGGCACCTCCACCGAACCA | AEAASASGEAGRSANH | |
| | TCGGAGGGCAGCGCCCCTGGGAGCCCTGCCGGGAGCCCTACAAGC | TPAGLTGPEAGRSANH | |
| | ACCGAAGAGGGCACCAGTACAGAGCCAAGTGAGGGGAGCGCCCCT | TPAGLTGPRVIPVSGP | |
| | GGTACTAGTACTGAACCATCCGAGGGGTCAGCTCCAGGCACGAGT | ARCLSQSRNLLKTTDD | |
| | GAGTCCGCTACCCCCGAGAGCGGACCGGGCTCAGAGCCCGCCACG | MVKTAREKLKHYSCTA | |
| | AGTGGCAGTGAAACTCCAGGCTCAGAACCCGCCACTAGTGGGTCA | EDIDHEDITRDQTSTL | |
| | GAGACTCCAGGCAGCCCTGCCGGATCCCCTACGTCCACCGAGGAG | KTCLPLELHKNESCLA | |
| | GGAACATCTGAGTCCGCAACACCCGAATCCGGTCCAGGCACCTCC | TRETSSTTRGSCLPPQ | |
| | ACGGAACCTAGTGAAGGCTCGGCACCAGGTACAAGCACCGAACCT | KTSLMMTLCLGSIYED | |
| | AGCGAGGGCAGCGCTCCCGGCAGCCCTGCCGGCAGCCCAACCTCA | LKMYQTEFQAINAALQ | |
| | ACTGAGGAGGGCACCAGTACTGAGCCCAGCGAGGGATCAGCACCT | NHNHQQIILDKGMLVA | |
| | GGCACCAGCACCGAACCTAGCGAGGGGAGCGCCCCTGGGACTAGC | IDELMQSLNHNGETLR | |
| | GAGTCAGCTACACCAGAGAGCGGGCCTGGAACTTCTACCGAACCC | QKPPVGEADPYRVKMK | |
| | AGTGAGGGATCCGCTCCAGGCACCTCCGAATCCGCAACCCCCGAA | LCILLHAFSTRVVTIN | |
| | TCCGGACCTGGCTCAGAGCCCGCCACCAGCGGGAGCGAAACCCCT | RVMGYLSSAGTSESAT | |
| | GGCACATCCACCGAGCCTAGCGAAGGGTCCGCACCCGGCACCAGT | PESGPGSEPATSGSET | |
| | ACAGAGCCTAGCGAGGGATCAGCACCTGGCACCAGTGAATCTGCT | PGTSESATPESGPGSE | |
| | ACACCAGAGAGCGGCCCTGGAACCTCCGAGTCCGCTACCCCCGAG | PATSGSETPGTSESAT | |
| | AGCGGGCCAGAGGCCGGCCGGAGCGCCAACCACACCCCCGCCGGC | PESGPGTSTEPSEGSA | |
| | CTGACCGGCCCTGGCACAGCCGAGGCCGCTAGCGCCAGCGGCAGA | PGSPAGSPTSTEEGTS | |
| | GTGATCCCCGTGAGCGGACCAGCAAGGTGCCTGTCCCAGAGCCGG | ESATPESGPGSEPATS | |
| | AACCTGCTGAAGACCACAGACGATATGGTGAAGACCGCCCGGGAG | GSETPGTSESATPESG | |
| | AAGCTGAAGCACTACTCTTGTACAGCCGAGGACATCGATCACGAG | PGSPAGSPTSTEEGSP | |
| | GACATCACCCGGGATCAGACCTCTACACTGAAGACATGCCTGCCC | AGSPTSTEEGTSTEPS | |
| | CTGGAGCTGCACAAGAACGAGAGCTGTCTGGCCACCCGGGAGACA | EGSAPGTSESATPESG | |
| | AGCTCCACCACAAGAGGCAGCTGCCTGCCCCCTCAGAAGACCTCC | PGTSESATPESGPGTS | |
| | CTGATGATGACCCTGTGCCTGGGCTCTATCTACGAGGACCTGAAG | ESATPESGPGSEPATS | |
| | ATGTATCAGACCGAGTTCCAGGCCATCAATGCCGCCCTGCAGAAC | GGSPAGSPTSTEEGTS | |
| | CACAATCACCAGCAGATCATCCTGGACAAGGGCATGCTGGTGGCC | ESATPESGPGTSTEPS | |
| | ATCGATGAGCTGATGCAGAGCCTGAACCACAATGGCGAGACCCTG | EGSAPGSPAGSPTSTE | |
| | AGGCAGAAGCCACCAGTGGGAGAGGCAGATCCTTACAGGGTGAAG | EGTSTEPSEGSAPGTS | |
| | ATGAAGCTGTGCATCCTGCTGCACGCCTTTTCCACCAGGGTGGTG | TEPSEGSAPGTSESAT | |
| | ACAATCAATCGCGTGATGGGCTATCTGTCTAGCGCCGGCACAGCC | PESGPGSEPATSGSET | |
| | GAGGCCGCTAGCGCCAGCGGCGAGGCCGGCCGGAGCGCCAACCAC | PGSEPATSGSETPGSP | |
| | ACCCCCGCCGGCCTGACCGGCCCTGGTACCTCAGAGTCTGCTACC | AGSPTSTEEGTSESAT | |
| | CCCGAGTCAGGGCCAGGATCAGAGCCAGCCACCTCCGGGTCTGAG | PESGPGTSTEPSEGSA | |
| | ACACCCGGGACTTCCGAGAGTGCCACCCCTGAGTCCGGACCCGGG | PGTSTEPSEGSAPGSP | |
| | TCCGAGCCCGCCACTTCCGGCTCCGAAACTCCCGGCACAAGCGAG | AGSPTSTEEGTSTEPS | |
| | AGCGCTACCCCAGAGTCAGGACCAGGAACATCTACAGAGCCCTCT | EGSAPGTSTEPSEGSA | |
| | GAAGGCTCCGCTCCAGGGTCCCCAGCCGGCAGTCCCACTAGCACC | PGTSESATPESGPGTS | |
| | GAGGAGGGAACCTCTGAAAGCGCCACACCCGAATCAGGGCCAGGG | TEPSEGSAPGTSESAT | |
| | TCTGAGCCTGCTACCAGCGGCAGCGAGACACCAGGCACCTCTGAG | PESGPGSEPATSGSET | |
| | TCCGCCACACCAGAGTCCGGACCCGGATCTCCCGCTGGGAGCCCC | PGTSTEPSEGSAPGTS | |
| | ACCTCCACTGAGGAGGGATCTCCTGCTGGCTCTCCAACATCTACT | TEPSEGSAPGTSESAT | |
| | GAGGAAGGTACCTCAACCGAGCCATCCGAGGGATCAGCTCCCGGC | PESGPGTSESATPESG | |
| | ACCTCAGAGTCGGCAACCCCGGAGTCTGGACCCGGAACTTCCGAA | P (SEQ ID NO: | |
| | AGTGCCACACCAGAGTCCGGTCCCGGGACTTCAGAATCAGCAACA | 856) | |
| | CCCGAGTCCGGCCCTGGGTCTGAACCCGCCACAAGTGGTGAACCT | | |
| | GAGGCC (SEQ ID NO: 838) | | |
| XP09 | GGTTCTCCAGCCGGGTCCCCAACTTCGACCGAGGAAGGGACCTCC | GSPAGSPTSTEEGTSE | P35 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGRVIPVS | |
| | CTGACCGGCCCTGGCACAGCCGAGGCCGCTAGCGCCAGCGGCAGA | GPARCLSQSRNLLKTT | |
| | GTGATCCCCGTGAGCGGACCAGCAAGGTGCCTGTCCCAGAGCCGG | DDMVKTAREKLKHYSC | |
| | AACCTGCTGAAGACCACAGACGATATGGTGAAGACCGCCCGGGAG | TAEDIDHEDITRDQTS | |
| | AAGCTGAAGCACTACTCTTGTACAGCCGAGGACATCGATCACGAG | TLKTCLPLELHKNESC | |
| | GACATCACCCGGGATCAGACCTCTACACTGAAGACATGCCTGCCC | LATRETSSTTRGSCLP | |
| | CTGGAGCTGCACAAGAACGAGAGCTGTCTGGCCACCCGGGAGACA | PQKTSLMMTLCLGSTY | |
| | AGCTCCACCACAAGAGGCAGCTGCCTGCCCCCTCAGAAGACCTCC | EDLKMYQTEFQAINAA | |
| | CTGATGATGACCCTGTGCCTGGGCTCTATCTACGAGGACCTGAAG | LQNHNHQQIILDKGML | |
| | ATGTATCAGACCGAGTTCCAGGCCATCAATGCCGCCCTGCAGAAC | VAIDELMQSLNHNGET | |
| | CACAATCACCAGCAGATCATCCTGGACAAGGGCATGCTGGTGGCC | LRQKPPVGEADPYRVK | |
| | ATCGATGAGCTGATGCAGAGCCTGAACCACAATGGCGAGACCCTG | MKLCILLHAFSTRVVT | |
| | AGGCAGAAGCCACCAGTGGGAGAGGCAGATCCTTACAGGGTGAAG | INRVMGYLSSAGTAEA | |
| | ATGAAGCTGTGCATCCTGCTGCACGCCTTTTCCACCAGGGTGGTG | ASASGEAGRSANHTPA | |
| | ACAATCAATCGCGTGATGGGCTATCTGTCTAGCGCCGGCACAGCC | GLTGPESGPGTSTEPS | |
| | GAGGCCGCTAGCGCCAGCGGCGAGGCCGGCCGGAGCGCCAACCAC | EGSAPGTSESATPESG | |
| | ACCCCCGCCGGCCTGACCGGCCCTGAGAGCGGCCCTGGCACCAGC | PGSPAGSPTSTEEGSP | |
| | ACCGAGCCATCCGAGGGATCCGCCCCAGGCACTTCTGAGTCAGCC | AGSPTSTEEGSPAGSP | |
| | ACACCCGAAAGCGGACCAGGATCACCCGCTGGCTCCCCCACCAGT | TSTEEGTSESATPESG | |
| | ACCGAGGAGGGGTCCCCCGCTGGAAGTCCAACAAGCACTGAGGAA | PGTSTEPSEGSAPGTS | |
| | GGGTCCCCTGCCGGCTCCCCCACAAGTACCGAAGAGGGCACAAGT | ESATPESGPGSEPATS | |
| | GAGAGCGCCACTCCCGAGTCCGGGCCTGGCACCAGCACAGAGCCT | GSETPGTSESATPESG | |
| | TCCGAGGGGTCCGCACCAGGTACCTCAGAGTCTGCTACCCCCGAG | PGSEPATSGSETPGTS | |
| | TCAGGGCCAGGATCAGAGCCAGCCACCTCCGGGTCTGAGACACCC | ESATPESGPGTSTEPS | |
| | GGGACTTCCGAGAGTGCCACCCCTGAGTCCGGACCCGGGTCCGAG | EGSAPGSPAGSPTSTE | |
| | CCCGCCACTTCCGGCTCCGAAACTCCCGGCACAAGCGAGAGCGCT | EGTSESATPESGPGSE | |
| | ACCCCAGAGTCAGGACCAGGAACATCTACAGAGCCCTCTGAAGGC | PATSGSETPGTSESAT | |
| | TCCGCTCCAGGGTCCCCAGCCGGCAGTCCCACTAGCACCGAGGAG | PESGPGSPAGSPTSTE | |
| | GGAACCTCTGAAAGCGCCACACCCGAATCAGGGCCAGGGTCTGAG | EGSPAGSPTSTEEGTS | |
| | CCTGCTACCAGCGGCAGCGAGACACCAGGCACCTCTGAGTCCGCC | TEPSEGSAPGTSESAT | |
| | ACACCAGAGTCCGGACCCGGATCTCCCGCTGGGAGCCCCACCTCC | PESGPGTSESATPESG | |
| | ACTGAGGAGGGATCTCCTGCTGGCTCTCCAACATCTACTGAGGAA | PGTSESATPESGPGSE | |
| | GGTACCTCAACCGAGCCATCCGAGGGATCAGCTCCCGGCACCTCA | PATSGSETPGSEPATS | |
| | GAGTCGGCAACCCCGGAGTCTGGACCCGGAACTTCCGAAAGTGCC | GSETPGSPAGSPTSTE | |
| | ACACCAGAGTCCGGTCCCGGGACTTCAGAATCAGCAACACCCGAG | EGTSTEPSEGSAPGTS | |
| | TCCGGCCCTGGGTCTGAACCCGCCACAAGTGGTAGTGAGACACCA | TEPSEGSAPGSEPATS | |
| | GGATCAGAACCTGCTACCTCAGGGTCAGAGACACCCGGATCTCCG | GSETPGTSESATPESG | |
| | GCAGGCTCACCAACCTCCACTGAGGAGGGCACCAGCACAGAACCA | PGTSTEPSEGSAPEPE | |
| | AGCGAGGGCTCCGCACCCGGAACAAGCACTGAACCCAGTGAGGGT | A (SEQ ID NO: | |
| | TCAGCACCCGGCTCTGAGCCGGCCACAAGTGGCAGTGAGACACCC | 857) | |
| | GGCACTTCAGAGAGTGCCACCCCCGAGAGTGGCCCAGGCACTAGT | | |
| | ACCGAGCCCTCTGAAGGCAGTGCGCCAGAACCTGAGGCC (SEQ | | |
| | ID NO: 839) | | |
| XP10 | GGTTCTCCAGCCGGGTCCCCAACTTCGACCGAGGAAGGGACCTCC | GSPAGSPTSTEEGTSE | P35 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGRVIPVS | |
| | CTGACCGGCCCTGGCACAGCCGAGGCCGCTAGCGCCAGCGGCAGA | GPARCLSQSRNLLKTT | |
| | GTGATCCCCGTGAGCGGACCAGCAAGGTGCCTGTCCCAGAGCCGG | DDMVKTAREKLKHYSC | |
| | AACCTGCTGAAGACCACAGACGATATGGTGAAGACCGCCCGGGAG | TAEDIDHEDITRDOTS | |
| | AAGCTGAAGCACTACTCTTGTACAGCCGAGGACATCGATCACGAG | TLKTCLPLELHKNESC | |
| | GACATCACCCGGGATCAGACCTCTACACTGAAGACATGCCTGCCC | LATRETSSTTRGSCLP | |
| | CTGGAGCTGCACAAGAACGAGAGCTGTCTGGCCACCCGGGAGACA | PQKTSLMMTLCLGSIY | |
| | AGCTCCACCACAAGAGGCAGCTGCCTGCCCCCTCAGAAGACCTCC | EDLKMYQTEFQAINAA | |
| | CTGATGATGACCCTGTGCCTGGGCTCTATCTACGAGGACCTGAAG | LQNHNHQQIILDKGML | |
| | ATGTATCAGACCGAGTTCCAGGCCATCAATGCCGCCCTGCAGAAC | VAIDELMQSLNHNGET | |
| | CACAATCACCAGCAGATCATCCTGGACAAGGGCATGCTGGTGGCC | LRQKPPVGEADPYRVK | |
| | ATCGATGAGCTGATGCAGAGCCTGAACCACAATGGCGAGACCCTG | MKLCILLHAFSTRVVT | |
| | AGGCAGAAGCCACCAGTGGGAGAGGCAGATCCTTACAGGGTGAAG | INRVMGYLSSAGTAEA | |
| | ATGAAGCTGTGCATCCTGCTGCACGCCTTTTCCACCAGGGTGGTG | ASASGEAGRSANHTPA | |
| | ACAATCAATCGCGTGATGGGCTATCTGTCTAGCGCCGGCACAGCC | GLTGPGTSESATPESG | |
| | GAGGCCGCTAGCGCCAGCGGCGAGGCCGGCCGGAGCGCCAACCAC | PGSEPATSGSETPGTS | |
| | ACCCCCGCCGGCCTGACCGGCCCTGGTACCTCAGAGTCTGCTACC | ESATPESGPGSEPATS | |
| | CCCGAGTCAGGGCCAGGATCAGAGCCAGCCACCTCCGGGTCTGAG | GSETPGTSESATPESG | |
| | ACACCCGGGACTTCCGAGAGTGCCACCCCTGAGTCCGGACCCGGG | PGTSTEPSEGSAPGSP | |
| | TCCGAGCCCGCCACTTCCGGCTCCGAAACTCCCGGCACAAGCGAG | AGSPTSTEEGTSESAT | |
| | AGCGCTACCCCAGAGTCAGGACCAGGAACATCTACAGAGCCCTCT | PESGPGSEPATSGSET | |
| | GAAGGCTCCGCTCCAGGGTCCCCAGCCGGCAGTCCCACTAGCACC | PGTSESATPESGPGSP | |
| | GAGGAGGGAACCTCTGAAAGCGCCACACCCGAATCAGGGCCAGGG | AGSPTSTEEGSPAGSP | |
| | TCTGAGCCTGCTACCAGCGGCAGCGAGACACCAGGCACCTCTGAG | TSTEEGTSTEPSEGSA | |
| | TCCGCCACACCAGAGTCCGGACCCGGATCTCCCGCTGGGAGCCCC | PGTSESATPESGPGTS | |
| | ACCTCCACTGAGGAGGGATCTCCTGCTGGCTCTCCAACATCTACT | ESATPESGPGTSESAT | |
| | GAGGAAGGTACCTCAACCGAGCCATCCGAGGGATCAGCTCCCGGC | PESGPGSEPATSGEPE | |
| | ACCTCAGAGTCGGCAACCCCGGAGTCTGGACCCGGAACTTCCGAA | A (SEQ ID NO: | |
| | AGTGCCACACCAGAGTCCGGTCCCGGGACTTCAGAATCAGCAACA | 858) | |
| | CCCGAGTCCGGCCCTGGGTCTGAACCCGCCACAAGTGGTGAACCT | | |
| | GAGGCCTAA (SEQ ID NO: 840) | | |
| XP11 | GGTTCTCCAGCCGGGTCCCCAACTTCGACCGAGGAAGGGACCTCC | GSPAGSPTSTEEGTSE | P35 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGRVIPVS | |
| | CTGACCGGCCCTGGCACAGCCGAGGCCGCTAGCGCCAGCGGCAGA | GPARCLSQSRNLLKTT | |
| | GTGATCCCCGTGAGCGGACCAGCAAGGTGCCTGTCCCAGAGCCGG | DDMVKTAREKLKHYSC | |
| | AACCTGCTGAAGACCACAGACGATATGGTGAAGACCGCCCGGGAG | TAEDIDHEDITRDQTS | |
| | AAGCTGAAGCACTACTCTTGTACAGCCGAGGACATCGATCACGAG | TLKTCLPLELHKNESC | |
| | GACATCACCCGGGATCAGACCTCTACACTGAAGACATGCCTGCCC | LATRETSSTTRGSCLP | |
| | CTGGAGCTGCACAAGAACGAGAGCTGTCTGGCCACCCGGGAGACA | PQKTSLMMTLCLGSIY | |
| | AGCTCCACCACAAGAGGCAGCTGCCTGCCCCCTCAGAAGACCTCC | EDLKMYQTEFQAINAA | |
| | CTGATGATGACCCTGTGCCTGGGCTCTATCTACGAGGACCTGAAG | LQNHNHQQIILDKGML | |
| | ATGTATCAGACCGAGTTCCAGGCCATCAATGCCGCCCTGCAGAAC | VAIDELMQSLNHNGET | |
| | CACAATCACCAGCAGATCATCCTGGACAAGGGCATGCTGGTGGCC | LRQKPPVGEADPYRVK | |
| | ATCGATGAGCTGATGCAGAGCCTGAACCACAATGGCGAGACCCTG | MKLCILLHAFSTRVVT | |
| | AGGCAGAAGCCACCAGTGGGAGAGGCAGATCCTTACAGGGTGAAG | INRVMGYLSSA (SEQ | |
| | ATGAAGCTGTGCATCCTGCTGCACGCCTTTTCCACCAGGGTGGTG | ID NO: 859) | |
| | ACAATCAATCGCGTGATGGGCTATCTGTCTAGCGCCTAA (SEQ | | |
| | ID NO: 841) | | |
| XP12 | GGTTCTCCAGCCGGGTCCCCAACTTCGACCGAGGAAGGGACCTCC | GSPAGSPTSTEEGTSE | P35 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGRVIPVS | |
| | CTGACCGGCCCTGGCACAGCCGAGGCCGCTAGCGCCAGCGGCAGA | GPARCLSQSRNLLKTT | |
| | GTGATCCCCGTGAGCGGACCAGCAAGGTGCCTGTCCCAGAGCCGG | DDMVKTAREKLKHYSC | |
| | AACCTGCTGAAGACCACAGACGATATGGTGAAGACCGCCCGGGAG | TAEDIDHEDITRDQTS | |
| | AAGCTGAAGCACTACTCTTGTACAGCCGAGGACATCGATCACGAG | TLKTCLPLELHKNESC | |
| | GACATCACCCGGGATCAGACCTCTACACTGAAGACATGCCTGCCC | LATRETSSTTRGSCLP | |
| | CTGGAGCTGCACAAGAACGAGAGCTGTCTGGCCACCCGGGAGACA | PQKTSLMMTLCLGSIY | |
| | AGCTCCACCACAAGAGGCAGCTGCCTGCCCCCTCAGAAGACCTCC | EDLKMYQTEFQAINAA | |
| | CTGATGATGACCCTGTGCCTGGGCTCTATCTACGAGGACCTGAAG | LQNHNHQQIILDKGML | |
| | ATGTATCAGACCGAGTTCCAGGCCATCAATGCCGCCCTGCAGAAC | VAIDELMQSLNHNGET | |
| | CACAATCACCAGCAGATCATCCTGGACAAGGGCATGCTGGTGGCC | LRQKPPVGEADPYRVK | |
| | ATCGATGAGCTGATGCAGAGCCTGAACCACAATGGCGAGACCCTG | MKLCILLHAFSTRVVT | |
| | AGGCAGAAGCCACCAGTGGGAGAGGCAGATCCTTACAGGGTGAAG | INRVMGYLSSAGTAEA | |
| | ATGAAGCTGTGCATCCTGCTGCACGCCTTTTCCACCAGGGTGGTG | ASASGVLQSPGTAEAA | |
| | ACAATCAATCGCGTGATGGGCTATCTGTCTAGCGCCGGCACAGCC | SASGEAGRSANHTPAG | |
| | GAGGCCGCTAGCGCCAGCGGCGTGCTGCAGAGCCCAGGCACAGCC | LTGPGTSESATPESGP | |
| | GAGGCCGCTAGCGCCAGCGGCGAGGCCGGCCGGAGCGCCAACCAC | GSEPATSGSETPGTSE | |
| | ACCCCCGCCGGCCTGACCGGCCCTGGTACCTCAGAGTCTGCTACC | SATPESGPGSEPATSG | |
| | CCCGAGTCAGGGCCAGGATCAGAGCCAGCCACCTCCGGGTCTGAG | SETPGTSESATPESGP | |
| | ACACCCGGGACTTCCGAGAGTGCCACCCCTGAGTCCGGACCCGGG | GTSTEPSEGSAPGSPA | |
| | TCCGAGCCCGCCACTTCCGGCTCCGAAACTCCCGGCACAAGCGAG | GSPTSTEEGTSESATP | |
| | AGCGCTACCCCAGAGTCAGGACCAGGAACATCTACAGAGCCCTCT | ESGPGSEPATSGSETP | |
| | GAAGGCTCCGCTCCAGGGTCCCCAGCCGGCAGTCCCACTAGCACC | GTSESATPESGPGSPA | |
| | GAGGAGGGAACCTCTGAAAGCGCCACACCCGAATCAGGGCCAGGG | GSPTSTEEGSPAGSPT | |
| | TCTGAGCCTGCTACCAGCGGCAGCGAGACACCAGGCACCTCTGAG | STEEGTSTEPSEGSAP | |
| | TCCGCCACACCAGAGTCCGGACCCGGATCTCCCGCTGGGAGCCCC | GTSESATPESGPGTSE | |
| | ACCTCCACTGAGGAGGGATCTCCTGCTGGCTCTCCAACATCTACT | SATPESGPGTSESATP | |
| | GAGGAAGGTACCTCAACCGAGCCATCCGAGGGATCAGCTCCCGGC | ESGPGSEPATSGSETP | |
| | ACCTCAGAGTCGGCAACCCCGGAGTCTGGACCCGGAACTTCCGAA | GSEPATSGSETPGSPA | |
| | AGTGCCACACCAGAGTCCGGTCCCGGGACTTCAGAATCAGCAACA | GSPTSTEEGTSTEPSE | |
| | CCCGAGTCCGGCCCTGGGTCTGAACCCGCCACAAGTGGTAGTGAG | GSAPGTSTEPSEGSAP | |
| | ACACCAGGATCAGAACCTGCTACCTCAGGGTCAGAGACACCCGGA | GSEPATSGSETPGTSE | |
| | TCTCCGGCAGGCTCACCAACCTCCACTGAGGAGGGCACCAGCACA | SATPESGPGTSTEPSE | |
| | GAACCAAGCGAGGGCTCCGCACCCGGAACAAGCACTGAACCCAGT | GSAP (SEQ ID NO: | |
| | GAGGGTTCAGCACCCGGCTCTGAGCCGGCCACAAGTGGCAGTGAG | 860) | |
| | ACACCCGGCACTTCAGAGAGTGCCACCCCCGAGAGTGGCCCAGGC | | |
| | ACTAGTACCGAGCCCTCTGAAGGCAGTGCGCCA (SEQ ID NO: | | |
| | 842) | | |
| XP13 | GGTTCTCCAGCCGGGTCCCCAACTTCGACCGAGGAAGGGACCTCC | GSPAGSPTSTEEGTSE | P35 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | EAGRSANHTPAGLTGP | |
| | AGCGGGCCAGAGGCCGGCCGGAGCGCCAACCACACCCCCGCCGGC | GTAEAASASGRVIPVS | |
| | CTGACCGGCCCTGGCACAGCCGAGGCCGCTAGCGCCAGCGGCAGA | GPARCLSQSRNLLKTT | |
| | GTGATCCCCGTGAGCGGACCAGCAAGGTGCCTGTCCCAGAGCCGG | DDMVKTAREKLKHYSC | |
| | AACCTGCTGAAGACCACAGACGATATGGTGAAGACCGCCCGGGAG | TAEDIDHEDITRDQTS | |
| | AAGCTGAAGCACTACTCTTGTACAGCCGAGGACATCGATCACGAG | TLKTCLPLELHKNESC | |
| | GACATCACCCGGGATCAGACCTCTACACTGAAGACATGCCTGCCC | LATRETSSTTRGSCLP | |
| | CTGGAGCTGCACAAGAACGAGAGCTGTCTGGCCACCCGGGAGACA | PQKTSLMMTLCLGSIY | |
| | AGCTCCACCACAAGAGGCAGCTGCCTGCCCCCTCAGAAGACCTCC | EDLKMYQTEFQAINAA | |
| | CTGATGATGACCCTGTGCCTGGGCTCTATCTACGAGGACCTGAAG | LQNHNHQQIILDKGML | |
| | ATGTATCAGACCGAGTTCCAGGCCATCAATGCCGCCCTGCAGAAC | VAIDELMQSLNHNGET | |
| | CACAATCACCAGCAGATCATCCTGGACAAGGGCATGCTGGTGGCC | LRQKPPVGEADPYRVK | |
| | ATCGATGAGCTGATGCAGAGCCTGAACCACAATGGCGAGACCCTG | MKLCILLHAFSTRVVT | |
| | AGGCAGAAGCCACCAGTGGGAGAGGCAGATCCTTACAGGGTGAAG | INRVMGYLSSAGTAEA | |
| | ATGAAGCTGTGCATCCTGCTGCACGCCTTTTCCACCAGGGTGGTG | ASASGEAGRSANHTPA | |
| | ACAATCAATCGCGTGATGGGCTATCTGTCTAGCGCCGGCACAGCC | GLTGPGTSESATPESG | |
| | GAGGCCGCTAGCGCCAGCGGCGAGGCCGGCCGGAGCGCCAACCAC | PGSEPATSGSETPGTS | |
| | ACCCCCGCCGGCCTGACCGGCCCTGGTACCTCAGAGTCTGCTACC | ESATPESGPGSEPATS | |
| | CCCGAGTCAGGGCCAGGATCAGAGCCAGCCACCTCCGGGTCTGAG | GSETPGTSESATPESG | |
| | ACACCCGGGACTTCCGAGAGTGCCACCCCTGAGTCCGGACCCGGG | PGTSTEPSEGSAPGSP | |
| | TCCGAGCCCGCCACTTCCGGCTCCGAAACTCCCGGCACAAGCGAG | AGSPTSTEEGTSESAT | |
| | AGCGCTACCCCAGAGTCAGGACCAGGAACATCTACAGAGCCCTCT | PESGPGSEPATSGSET | |
| | GAAGGCTCCGCTCCAGGGTCCCCAGCCGGCAGTCCCACTAGCACC | PGTSESATPESGPGSP | |
| | GAGGAGGGAACCTCTGAAAGCGCCACACCCGAATCAGGGCCAGGG | AGSPTSTEEGSPAGSP | |
| | TCTGAGCCTGCTACCAGCGGCAGCGAGACACCAGGCACCTCTGAG | TSTEEGTSTEPSEGSA | |
| | TCCGCCACACCAGAGTCCGGACCCGGATCTCCCGCTGGGAGCCCC | PGTSESATPESGPGTS | |
| | ACCTCCACTGAGGAGGGATCTCCTGCTGGCTCTCCAACATCTACT | ESATPESGPGTSESAT | |
| | GAGGAAGGTACCTCAACCGAGCCATCCGAGGGATCAGCTCCCGGC | PESGPGSEPATSGSET | |
| | ACCTCAGAGTCGGCAACCCCGGAGTCTGGACCCGGAACTTCCGAA | PGSEPATSGSETPGSP | |
| | AGTGCCACACCAGAGTCCGGTCCCGGGACTTCAGAATCAGCAACA | AGSPTSTEEGTSTEPS | |
| | CCCGAGTCCGGCCCTGGGTCTGAACCCGCCACAAGTGGTAGTGAG | EGSAPGTSTEPSEGSA | |
| | ACACCAGGATCAGAACCTGCTACCTCAGGGTCAGAGACACCCGGA | PGSEPATSGSETPGTS | |
| | TCTCCGGCAGGCTCACCAACCTCCACTGAGGAGGGCACCAGCACA | ESATPESGPGTSTEPS | |
| | GAACCAAGCGAGGGCTCCGCACCCGGAACAAGCACTGAACCCAGT | EGSAP (SEQ ID | |
| | GAGGGTTCAGCACCCGGCTCTGAGCCGGCCACAAGTGGCAGTGAG | NO: 861) | |
| | ACACCCGGCACTTCAGAGAGTGCCACCCCCGAGAGTGGCCCAGGC | | |
| | ACTAGTACCGAGCCCTCTGAAGGCAGTGCGCCA (SEQ ID NO: | | |
| | 843) | | |
| XP14 | GGTTCTCCAGCCGGGTCCCCAACTTCGACCGAGGAAGGGACCTCC | GSPAGSPTSTEEGTSE | P35 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | GSPAGSPTSTEEGTSE | |
| | AGCGGGCCAGGTTCTCCTGCTGGCTCCCCCACCTCAACAGAAGAG | SATPESGPGSEPATSG | |
| | GGGACAAGCGAAAGCGCTACGCCTGAGAGTGGCCCTGGCTCTGAG | SETPGTSESATPESGP | |
| | CCAGCCACCTCCGGCTCTGAAACCCCTGGCACTAGTGAGTCTGCC | GTSTEPSEGSAPGTST | |
| | ACGCCTGAGTCCGGACCCGGGACCTCTACTGAGCCCTCGGAGGGG | EPSEGSAPGTSTEPSE | |
| | AGCGCTCCTGGCACGAGTACAGAACCTTCCGAAGGAAGTGCACCG | GSAPGTSTEPSEGSAP | |
| | GGCACAAGCACCGAGCCTTCCGAAGGCTCTGCTCCCGGAACCTCT | GTSTEPSEGSAPGTST | |
| | ACCGAACCCTCTGAAGGGTCTGCACCCGGCACGAGCACCGAACCC | EPSEGSAPGSPAGSPT | |
| | AGCGAAGGGTCAGCGCCTGGGACCTCAACAGAGCCCTCGGAAGGA | STEEGTSTEPSEGSAP | |
| | TCAGCGCCTGGAAGCCCTGCAGGGAGTCCAACTTCCACGGAAGAA | EAGRSANHTPAGLTGP | |
| | GGAACGTCTACAGAGCCATCAGAGGGGTCCGCACCAGAGGCCGGC | GTAEAASASGRVIPVS | |
| | CGGAGCGCCAACCACACCCCCGCCGGCCTGACCGGCCCTGGCACA | GPARCLSQSRNLLKTT | |
| | GCCGAGGCCGCTAGCGCCAGCGGCAGAGTGATCCCCGTGAGCGGA | DDMVKTAREKLKHYSC | |
| | CCAGCAAGGTGCCTGTCCCAGAGCCGGAACCTGCTGAAGACCACA | TAEDIDHEDITRDQTS | |
| | GACGATATGGTGAAGACCGCCCGGGAGAAGCTGAAGCACTACTCT | TLKTCLPLELHKNESC | |
| | TGTACAGCCGAGGACATCGATCACGAGGACATCACCCGGGATCAG | LATRETSSTTRGSCLP | |
| | ACCTCTACACTGAAGACATGCCTGCCCCTGGAGCTGCACAAGAAC | PQKTSLMMTLCLGSIY | |
| | GAGAGCTGTCTGGCCACCCGGGAGACAAGCTCCACCACAAGAGGC | EDLKMYQTEFQAINAA | |
| | AGCTGCCTGCCCCCTCAGAAGACCTCCCTGATGATGACCCTGTGC | LQNHNHQQIILDKGML | |
| | CTGGGCTCTATCTACGAGGACCTGAAGATGTATCAGACCGAGTTC | VAIDELMQSLNHNGET | |
| | CAGGCCATCAATGCCGCCCTGCAGAACCACAATCACCAGCAGATC | LRQKPPVGEADPYRVK | |
| | ATCCTGGACAAGGGCATGCTGGTGGCCATCGATGAGCTGATGCAG | MKLCILLHAFSTRVVT | |
| | AGCCTGAACCACAATGGCGAGACCCTGAGGCAGAAGCCACCAGTG | INRVMGYLSSAGTAEA | |
| | GGAGAGGCAGATCCTTACAGGGTGAAGATGAAGCTGTGCATCCTG | ASASGEAGRSANHTPA | |
| | CTGCACGCCTTTTCCACCAGGGTGGTGACAATCAATCGCGTGATG | GLTGPGTSESATPESG | |
| | GGCTATCTGTCTAGCGCCGGCACAGCCGAGGCCGCTAGCGCCAGC | PGSEPATSGSETPGTS | |
| | GGCGAGGCCGGCCGGAGCGCCAACCACACCCCCGCCGGCCTGACC | ESATPESGPGSEPATS | |
| | GGCCCTGGTACCAGCGAATCCGCTACTCCCGAATCTGGCCCTGGG | GSETPGTSESATPESG | |
| | TCCGAACCTGCCACCTCCGGCTCTGAAACTCCAGGGACCTCCGAA | PGTSTEPSEGSAPGTS | |
| | TCTGCCACACCCGAGAGCGGCCCTGGCTCCGAGCCCGCAACATCT | ESATPESGPGSPAGSP | |
| | GGCAGCGAGACACCTGGCACCTCCGAGAGCGCAACACCCGAGAGC | TSTEEGSPAGSPTSTE | |
| | GGCCCTGGCACCAGCACCGAGCCATCCGAGGGATCCGCCCCAGGC | EGSPAGSPTSTEEGTS | |
| | ACTTCTGAGTCAGCCACACCCGAAAGCGGACCAGGATCACCCGCT | ESATPESGPGTSTEPS | |
| | GGCTCCCCCACCAGTACCGAGGAGGGGTCCCCCGCTGGAAGTCCA | EGSAPGTSESATPESG | |
| | ACAAGCACTGAGGAAGGGTCCCCTGCCGGCTCCCCCACAAGTACC | PGSEPATSGSETPGTS | |
| | GAAGAGGGCACAAGTGAGAGCGCCACTCCCGAGTCCGGGCCTGGC | ESATPESGPGSEPATS | |
| | ACCAGCACAGAGCCTTCCGAGGGGTCCGCACCAGGTACCTCAGAG | GSETPGTSESATPESG | |
| | TCTGCTACCCCCGAGTCAGGGCCAGGATCAGAGCCAGCCACCTCC | PGTSTEPSEGSAPGSP | |
| | GGGTCTGAGACACCCGGGACTTCCGAGAGTGCCACCCCTGAGTCC | AGSPTSTEEGTSESAT | |
| | GGACCCGGGTCCGAGCCCGCCACTTCCGGCTCCGAAACTCCCGGC | PESGPGSEPATSGSET | |
| | ACAAGCGAGAGCGCTACCCCAGAGTCAGGACCAGGAACATCTACA | PGTSESATPESGPGSP | |
| | GAGCCCTCTGAAGGCTCCGCTCCAGGGTCCCCAGCCGGCAGTCCC | AGSPTSTEEGSPAGSP | |
| | ACTAGCACCGAGGAGGGAACCTCTGAAAGCGCCACACCCGAATCA | TSTEEGTSTEPSEGSA | |
| | GGGCCAGGGTCTGAGCCTGCTACCAGCGGCAGCGAGACACCAGGC | PGTSESATPESGPGTS | |
| | ACCTCTGAGTCCGCCACACCAGAGTCCGGACCCGGATCTCCCGCT | ESATPESGPGTSESAT | |
| | GGGAGCCCCACCTCCACTGAGGAGGGATCTCCTGCTGGCTCTCCA | PESGPGSEPATSGSET | |
| | ACATCTACTGAGGAAGGTACCTCAACCGAGCCATCCGAGGGATCA | PGSEPATSGSETPGSP | |
| | GCTCCCGGCACCTCAGAGTCGGCAACCCCGGAGTCTGGACCCGGA | AGSPTSTEEGTSTEPS | |
| | ACTTCCGAAAGTGCCACACCAGAGTCCGGTCCCGGGACTTCAGAA | EGSAPGTSTEPSEGSA | |
| | TCAGCAACACCCGAGTCCGGCCCTGGGTCTGAACCCGCCACAAGT | PGSEPATSGSETPGTS | |
| | GGTAGTGAGACACCAGGATCAGAACCTGCTACCTCAGGGTCAGAG | ESATPESGPGTSTEPS | |
| | ACACCCGGATCTCCGGCAGGCTCACCAACCTCCACTGAGGAGGGC | EGSAP (SEQ ID | |
| | ACCAGCACAGAACCAAGCGAGGGCTCCGCACCCGGAACAAGCACT | NO: 862) | |
| | GAACCCAGTGAGGGTTCAGCACCCGGCTCTGAGCCGGCCACAAGT | | |
| | GGCAGTGAGACACCCGGCACTTCAGAGAGTGCCACCCCCGAGAGT | | |
| | GGCCCAGGCACTAGTACCGAGCCCTCTGAAGGCAGTGCGCCA | | |
| | (SEQ ID NO: 844) | | |
| XP15 | GCAAGCTCCGCCACCCCTGAGTCTGGACCAGGCACCAGCACAGAG | ASSATPESGPGTSTEP | P35 |
| | CCTTCCGAGGGATCTGCCCCAGGCACCAGCGAGTCCGCCACACCA | SEGSAPGTSESATPES | |
| | GAGTCCGGACCTGGATCTGGACCAGGCACCTCTGAGAGCGCCACC | GPGSGPGTSESATPGT | |
| | CCAGGCACATCCGAGTCTGCCACCCCAGAGAGCGGACCTGGATCC | SESATPESGPGSEPAT | |
| | GAGCCAGCCACAAGCGGATCCGAGACCCCAGGCACATCTGAAAGC | SGSETPGTSESATPES | |
| | GCCACTCCAGAGTCCGGACCTGGCACCTCTACAGAGCCTAGCGAG | GPGTSTEPSEGSAPGS | |
| | GGATCCGCCCCTGGAAGCCCAGCCGGCTCTCCTACCAGCACAGAG | PAGSPTSTEEGTSESA | |
| | GAGGGCACCTCCGAGTCTGCCACACCAGAGTCTGGACCAGGAAGC | TPESGPGSEPATSGSE | |
| | GAGCCTGCCACCAGCGGCAGCGAAACTCCAGGCACATCTGAGAGC | TPGTSESATPESGPGS | |
| | GCCACCCCTGAGTCCGGACCAGGATCTCCTGCAGGATCCCCTACC | PAGSPTSTEEGSPAGS | |
| | TCTACAGAGGAGGGAAGCCCAGCAGGAAGCCCCACCTCCACCGAA | PTSTEEGTSTEPSEGS | |
| | GAGGGCACCTCCACAGAGCCATCTGAGGGAAGCGCCCCTGGCACC | APGTSESATPESGPGT | |
| | TCCGAATCTGCCACACCTGAGTCCGGACCCGGCACCAGCGAATCC | SESATPESGPGTSESA | |
| | GCCACCCCCGAGTCTGGACCTGGCACCTCTGAAAGCGCCACACCA | TPESGPGSEPATSGSE | |
| | GAGAGCGGACCAGGATCCGAGCCTGCCACCTCCGGATCTGAGACA | TPGSEPATSGSETPGS | |
| | CCAGGAAGCGAGCCAGCCACCAGCGGATCCGAGACACCAGGCTCC | PAGSPTSTEEGTSTEP | |
| | CCCGCCGGCTCCCCCACCTCTACAGAGGAGGGCACCAGCACCGAA | SEGSAPGTSTEPSEGS | |
| | CCTTCCGAGGGATCCGCCCCCGGCACCAGCACCGAGCCTTCCGAA | APGSEPATSGSETPGT | |
| | GGAAGCGCCCCAGGCTCCGAGCCAGCCACCTCTGGAAGTGAAACT | SESATPEAGRSANHTP | |
| | CCTGGCACATCCGAATCTGCCACCCCAGAGGCAGGCAGGTCCGCC | AGLTGPGTSESATPES | |
| | AACCACACACCAGCAGGACTGACCGGACCAGGCACAAGCGAGTCC | RVIPVSGPARCLSQSR | |
| | GCCACCCCAGAGAGCCGCGTGATCCCCGTGTCCGGACCTGCAAGG | NLLKTTDDMVKTAREK | |
| | TGCCTGTCTCAGAGCAGAAATCTGCTGAAGACCACAGACGATATG | LKHYSCTAEDIDHEDI | |
| | GTGAAGACCGCCCGGGAGAAGCTGAAGCACTACAGCTGTACAGCC | TRDQTSTLKTCLPLEL | |
| | GAGGACATCGATCACGAGGACATCACCAGAGATCAGACCAGCACA | HKNESCLATRETSSTT | |
| | CTGAAGACATGCCTGCCCCTGGAGCTGCACAAGAACGAGTCCTGT | RGSCLPPQKTSLMMTL | |
| | CTGGCCACCCGGGAGACATCTAGCACCACAAGAGGCTCTTGCCTG | CLGSIYEDLKMYQTEF | |
| | CCCCCTCAGAAGACCAGCCTGATGATGACCCTGTGCCTGGGCAGC | QAINAALQNHNHQQII | |
| | ATCTACGAGGACCTGAAGATGTATCAGACCGAGTTCCAGGCCATC | LDKGMLVAIDELMQSL | |
| | AATGCCGCCCTGCAGAACCACAATCACCAGCAGATCATCCTGGAC | NHNGETLRQKPPVGEA | |
| | AAGGGCATGCTGGTGGCCATCGATGAGCTGATGCAGAGCCTGAAC | DPYRVKMKLCILLHAF | |
| | CACAATGGCGAGACCCTGAGGCAGAAGCCACCAGTGGGAGAGGCA | STRVVTINRVMGYLSS | |
| | GATCCATACCGCGTGAAGATGAAGCTGTGCATCCTGCTGCACGCC | AGTATPESGPGEAGRS | |
| | TTTTCCACCAGGGTGGTGACAATCAACCGCGTGATGGGCTATCTG | ANHTPAGLTGPATPES | |
| | TCCTCTGCCGGCACTGCTACACCAGAGTCCGGACCAGGAGAGGCA | GPGSEPATSGSETPGT | |
| | GGCCGGTCTGCCAATCACACCCCTGCCGGACTGACCGGACCTGCA | SESATPESGPGSPAGS | |
| | ACACCAGAGTCTGGACCTGGCTCTGAGCCAGCCACCTCCGGCTCC | PTSTEEGSPAGSPTST | |
| | GAGACCCCTGGCACAAGCGAATCCGCCACCCCAGAAAGCGGCCCT | EEGTSTEPSEGSAPGT | |
| | GGCTCCCCAGCCGGCAGCCCTACCTCTACCGAAGAAGGCAGCCCA | SESATPESGPGTSESA | |
| | GCAGGAAGCCCTACCTCCACCGAGGAAGGCACCTCCACAGAGCCT | TPESGPGTSASATPES | |
| | TCTGAGGGAAGCGCCCCCGGCACCTCTGAAAGCGCCACGCCAGAA | GPGSEPATSGSETPGS | |
| | TCCGGACCAGGCACCTCCGAGTCTGCCACGCCTGAGTCCGGACCA | EPATSGSETPGSPAGS | |
| | GGCACCAGCGCCTCCGCCACACCCGAGAGCGGCCCAGGGAGCGAA | PTSTEEGTSTEPSEGS | |
| | CCAGCCACCTCTGGAAGCGAAACCCCTGGCAGTGAACCAGCCACC | APGTSTEPSEGSAPGS | |
| | TCCGGCTCTGAGACACCAGGATCCCCAGCCGGCTCACCTACCTCT | EPATSGSETPGTSESA | |
| | ACCGAGGAGGGCACCAGCACTGAACCTAGTGAGGGATCCGCCCCA | G (SEQ ID NO: | |
| | GGCACCTCTACCGAACCTAGCGAAGGCAGCGCCCCTGGCTCAGAG | 863) | |
| | CCAGCCACCAGCGGCAGCGAGACTCCTGGCACATCTGAAAGCGCC | | |
| | GGC (SEQ ID NO: 845) | | |
| XP16 | ATGTGGGAGCTGGAGAAGGACGTGTACGTGGTGGAGGTGGACTGG | MWELEKDVYVVEVDWT | IL12 |
| | ACACCAGATGCCCCCGGCGAGACCGTGAACCTGACATGCGACACC | PDAPGETVNLTCDTPE | |
| | CCCGAGGAGGACGATATCACCTGGACATCTGATCAGAGGCACGGC | EDDITWTSDQRHGVIG | |
| | GTGATCGGAAGCGGCAAGACCCTGACAATCACCGTGAAGGAGTTC | SGKTLTITVKEFLDAG | |
| | CTGGATGCCGGCCAGTACACATGTCACAAGGGCGGCGAGACCCTG | QYTCHKGGETLSHSHL | |
| | TCCCACTCTCACCTGCTGCTGCACAAGAAGGAGAACGGCATCTGG | LLHKKENGIWSTEILK | |
| | TCCACAGAGATCCTGAAGAACTTCAAGAATAAGACCTTTCTGAAG | NFKNKTFLKCEAPNYS | |
| | TGCGAGGCCCCTAATTATAGCGGCCGGTTCACCTGTTCCTGGCTG | GRFTCSWLVQRNMDLK | |
| | GTGCAGAGAAACATGGACCTGAAGTTTAATATCAAGAGCTCCTCT | FNIKSSSSSPDSRAVT | |
| | AGCTCCCCAGATAGCCGGGCAGTGACATGCGGAATGGCCAGCCTG | CGMASLSAEKVTLDQR | |
| | TCCGCCGAGAAGGTGACCCTGGACCAGAGAGATTACGAGAAGTAT | DYEKYSVSCQEDVTCP | |
| | TCTGTGAGCTGCCAGGAGGACGTGACATGTCCCACCGCCGAGGAG | TAEETLPIELALEARQ | |
| | ACACTGCCTATCGAGCTGGCCCTGGAGGCCAGGCAGCAGAACAAG | QNKYENYSTSFFIRDI | |
| | TACGAGAATTATTCCACCTCTTTCTTTATCCGCGACATCATCAAG | IKPDPPKNLQMKPLKN | |
| | CCAGATCCCCCTAAGAACCTGCAGATGAAGCCCCTGAAGAATTCC | SQVEVSWEYPDSWSTP | |
| | CAGGTCGAGGTGTCTTGGGAGTACCCTGACAGCTGGTCCACACCA | HSYFSLKFFVRIQRKK | |
| | CACTCTTATTTCAGCCTGAAGTTCTTTGTGAGGATCCAGCGCAAG | EKMKETEEGCNQKGAF | |
| | AAGGAGAAGATGAAGGAGACCGAGGAGGGCTGCAATCAGAAGGGC | LVEKTSTEVQCKGGNV | |
| | GCCTTTCTGGTGGAGAAGACATCCACCGAGGTGCAGTGCAAGGGA | CVQAQDRYYNSSCSKW | |
| | GGAAACGTGTGCGTGCAGGCACAGGATCGGTACTATAATTCTAGC | ACVPCRVRSGTAEAAS | |
| | TGTTCCAAGTGGGCCTGCGTGCCTTGTCGGGTGAGATCTGGCGGC | ASGEAGRSANHTPAGL | |
| | GGCGGCTCTGGCGGCGGCGGCTCCGGCGGCGGCGGCTCCAGAGTG | TGPGSPAGSPTSTEEG | |
| | ATCCCCGTGAGCGGACCAGCAAGGTGCCTGTCCCAGAGCCGGAAC | TSESATPESGPGTSTE | |
| | CTGCTGAAGACCACAGACGATATGGTGAAGACCGCCCGGGAGAAG | PSEGSAPGSPAGSPTS | |
| | CTGAAGCACTACTCTTGTACAGCCGAGGACATCGATCACGAGGAC | TEEGTSTEPSEGSAPG | |
| | ATCACCCGGGATCAGACCTCTACACTGAAGACATGCCTGCCCCTG | TSTEPSEGSAPGTSES | |
| | GAGCTGCACAAGAACGAGAGCTGTCTGGCCACCCGGGAGACAAGC | ATPESGPGSEPATSGS | |
| | TCCACCACAAGAGGCAGCTGCCTGCCCCCTCAGAAGACCTCCCTG | ETPGSEPATSGSETPG | |
| | ATGATGACCCTGTGCCTGGGCTCTATCTACGAGGACCTGAAGATG | SPAGSPTSTEEGTSES | |
| | TATCAGACCGAGTTCCAGGCCATCAATGCCGCCCTGCAGAACCAC | ATPESGPGTSTEPSEG | |
| | AATCACCAGCAGATCATCCTGGACAAGGGCATGCTGGTGGCCATC | SAPGTSTEPSEGSAPG | |
| | GATGAGCTGATGCAGAGCCTGAACCACAATGGCGAGACCCTGAGG | SPAGSPTSTEEGTSTE | |
| | CAGAAGCCACCAGTGGGAGAGGCAGATCCTTACAGGGTGAAGATG | PSEGSAPGTSTEPSEG | |
| | AAGCTGTGCATCCTGCTGCACGCCTTTTCCACCAGGGTGGTGACA | SAPGTSESATPESGPG | |
| | ATCAATCGCGTGATGGGCTATCTGTCTAGCGCCGGCACAGCCGAG | TSTEPSEGSAPGTSES | |
| | GCCGCTAGCGCCAGCGGCGAGGCCGGCCGGAGCGCCAACCACACC | ATPESGPGSEPATSGS | |
| | CCCGCCGGCCTGACCGGCCCTGGTTCTCCAGCCGGGTCCCCAACT | ETPGTSTEPSEGSAPG | |
| | TCGACCGAGGAAGGGACCTCCGAGTCAGCTACCCCGGAGTCCGGT | TSTEPSEGSAPGTSES | |
| | CCTGGCACCTCCACCGAACCATCGGAGGGCAGCGCCCCTGGGAGC | ATPESGPGTSESATPE | |
| | CCTGCCGGGAGCCCTACAAGCACCGAAGAGGGCACCAGTACAGAG | SGPGSPAGSPTSTEEG | |
| | CCAAGTGAGGGGAGCGCCCCTGGTACTAGTACTGAACCATCCGAG | TSESATPESGPGSEPA | |
| | GGGTCAGCTCCAGGCACGAGTGAGTCCGCTACCCCCGAGAGCGGA | TSGSETPGTSESATPE | |
| | CCGGGCTCAGAGCCCGCCACGAGTGGCAGTGAAACTCCAGGCTCA | SGPGTSTEPSEGSAPG | |
| | GAACCCGCCACTAGTGGGTCAGAGACTCCAGGCAGCCCTGCCGGA | TSTEPSEGSAPGTSTE | |
| | TCCCCTACGTCCACCGAGGAGGGAACATCTGAGTCCGCAACACCC | PSEGSAPGTSTEPSEG | |
| | GAATCCGGTCCAGGCACCTCCACGGAACCTAGTGAAGGCTCGGCA | SAPGTSTEPSEGSAPG | |
| | CCAGGTACAAGCACCGAACCTAGCGAGGGCAGCGCTCCCGGCAGC | TSTEPSEGSAPGSPAG | |
| | CCTGCCGGCAGCCCAACCTCAACTGAGGAGGGCACCAGTACTGAG | SPTSTEEGTSTEPSEG | |
| | CCCAGCGAGGGATCAGCACCTGGCACCAGCACCGAACCTAGCGAG | SAPGTSESATPESGPG | |
| | GGGAGCGCCCCTGGGACTAGCGAGTCAGCTACACCAGAGAGCGGG | SEPATSGSETPGTSES | |
| | CCTGGAACTTCTACCGAACCCAGTGAGGGATCCGCTCCAGGCACC | ATPESGPGSEPATSGS | |
| | TCCGAATCCGCAACCCCCGAATCCGGACCTGGCTCAGAGCCCGCC | ETPGTSESATPESGPG | |
| | ACCAGCGGGAGCGAAACCCCTGGCACATCCACCGAGCCTAGCGAA | TSTEPSEGSAPGTSES | |
| | GGGTCCGCACCCGGCACCAGTACAGAGCCTAGCGAGGGATCAGCA | ATPESGPGSPAGSPTS | |
| | CCTGGCACCAGTGAATCTGCTACACCAGAGAGCGGCCCTGGAACC | TEEGSPAGSPTSTEEG | |
| | TCCGAGTCCGCTACCCCCGAGAGCGGGCCAGGTTCTCCTGCTGGC | SPAGSPTSTEEGTSES | |
| | TCCCCCACCTCAACAGAAGAGGGGACAAGCGAAAGCGCTACGCCT | ATPESGPGTSTEPSEG | |
| | GAGAGTGGCCCTGGCTCTGAGCCAGCCACCTCCGGCTCTGAAACC | SAPGTSESATPESGPG | |
| | CCTGGCACTAGTGAGTCTGCCACGCCTGAGTCCGGACCCGGGACC | SEPATSGSETPGTSES | |
| | TCTACTGAGCCCTCGGAGGGGAGCGCTCCTGGCACGAGTACAGAA | ATPESGPGSEPATSGS | |
| | CCTTCCGAAGGAAGTGCACCGGGCACAAGCACCGAGCCTTCCGAA | ETPGTSESATPESGPG | |
| | GGCTCTGCTCCCGGAACCTCTACCGAACCCTCTGAAGGGTCTGCA | TSTEPSEGSAPGSPAG | |
| | CCCGGCACGAGCACCGAACCCAGCGAAGGGTCAGCGCCTGGGACC | SPTSTEEGTSESATPE | |
| | TCAACAGAGCCCTCGGAAGGATCAGCGCCTGGAAGCCCTGCAGGG | SGPGSEPATSGSETPG | |
| | AGTCCAACTTCCACGGAAGAAGGAACGTCTACAGAGCCATCAGAG | TSESATPESGPGSPAG | |
| | GGGTCCGCACCAGGTACCAGCGAATCCGCTACTCCCGAATCTGGC | SPTSTEEGSPAGSPTS | |
| | CCTGGGTCCGAACCTGCCACCTCCGGCTCTGAAACTCCAGGGACC | TEEGTSTEPSEGSAPG | |
| | TCCGAATCTGCCACACCCGAGAGCGGCCCTGGCTCCGAGCCCGCA | TSESATPESGPGTSES | |
| | ACATCTGGCAGCGAGACACCTGGCACCTCCGAGAGCGCAACACCC | ATPESGPGTSESATPE | |
| | GAGAGCGGCCCTGGCACCAGCACCGAGCCATCCGAGGGATCCGCC | SGPGSEPATSGSETPG | |
| | CCAGGCACTTCTGAGTCAGCCACACCCGAAAGCGGACCAGGATCA | SEPATSGSETPGSPAG | |
| | CCCGCTGGCTCCCCCACCAGTACCGAGGAGGGGTCCCCCGCTGGA | SPTSTEEGTSTEPSEG | |
| | AGTCCAACAAGCACTGAGGAAGGGTCCCCTGCCGGCTCCCCCACA | SAPGTSTEPSEGSAPG | |
| | AGTACCGAAGAGGGCACAAGTGAGAGCGCCACTCCCGAGTCCGGG | SEPATSGSETPGTSES | |
| | CCTGGCACCAGCACAGAGCCTTCCGAGGGGTCCGCACCAGGTACC | ATPESGPGTSTEPSEG | |
| | TCAGAGTCTGCTACCCCCGAGTCAGGGCCAGGATCAGAGCCAGCC | SAP (SEQ ID NO: | |
| | ACCTCCGGGTCTGAGACACCCGGGACTTCCGAGAGTGCCACCCCT | 864) | |
| | GAGTCCGGACCCGGGTCCGAGCCCGCCACTTCCGGCTCCGAAACT | | |
| | CCCGGCACAAGCGAGAGCGCTACCCCAGAGTCAGGACCAGGAACA | | |
| | TCTACAGAGCCCTCTGAAGGCTCCGCTCCAGGGTCCCCAGCCGGC | | |
| | AGTCCCACTAGCACCGAGGAGGGAACCTCTGAAAGCGCCACACCC | | |
| | GAATCAGGGCCAGGGTCTGAGCCTGCTACCAGCGGCAGCGAGACA | | |
| | CCAGGCACCTCTGAGTCCGCCACACCAGAGTCCGGACCCGGATCT | | |
| | CCCGCTGGGAGCCCCACCTCCACTGAGGAGGGATCTCCTGCTGGC | | |
| | TCTCCAACATCTACTGAGGAAGGTACCTCAACCGAGCCATCCGAG | | |
| | GGATCAGCTCCCGGCACCTCAGAGTCGGCAACCCCGGAGTCTGGA | | |
| | CCCGGAACTTCCGAAAGTGCCACACCAGAGTCCGGTCCCGGGACT | | |
| | TCAGAATCAGCAACACCCGAGTCCGGCCCTGGGTCTGAACCCGCC | | |
| | ACAAGTGGTAGTGAGACACCAGGATCAGAACCTGCTACCTCAGGG | | |
| | TCAGAGACACCCGGATCTCCGGCAGGCTCACCAACCTCCACTGAG | | |
| | GAGGGCACCAGCACAGAACCAAGCGAGGGCTCCGCACCCGGAACA | | |
| | AGCACTGAACCCAGTGAGGGTTCAGCACCCGGCTCTGAGCCGGCC | | |
| | ACAAGTGGCAGTGAGACACCCGGCACTTCAGAGAGTGCCACCCCC | | |
| | GAGAGTGGCCCAGGCACTAGTACCGAGCCCTCTGAAGGCAGTGCG | | |
| | CCA (SEQ ID NO: 846) | | |
| XP17 | GGTTCTCCAGCCGGGTCCCCAACTTCGACCGAGGAAGGGACCTCC | GSPAGSPTSTEEGTSE | IL12 |
| | GAGTCAGCTACCCCGGAGTCCGGTCCTGGCACCTCCACCGAACCA | SATPESGPGTSTEPSE | |
| | TCGGAGGGCAGCGCCCCTGGGAGCCCTGCCGGGAGCCCTACAAGC | GSAPGSPAGSPTSTEE | |
| | ACCGAAGAGGGCACCAGTACAGAGCCAAGTGAGGGGAGCGCCCCT | GTSTEPSEGSAPGTST | |
| | GGTACTAGTACTGAACCATCCGAGGGGTCAGCTCCAGGCACGAGT | EPSEGSAPGTSESATP | |
| | GAGTCCGCTACCCCCGAGAGCGGACCGGGCTCAGAGCCCGCCACG | ESGPGSEPATSGSETP | |
| | AGTGGCAGTGAAACTCCAGGCTCAGAACCCGCCACTAGTGGGTCA | GSEPATSGSETPGSPA | |
| | GAGACTCCAGGCAGCCCTGCCGGATCCCCTACGTCCACCGAGGAG | GSPTSTEEGTSESATP | |
| | GGAACATCTGAGTCCGCAACACCCGAATCCGGTCCAGGCACCTCC | ESGPGTSTEPSEGSAP | |
| | ACGGAACCTAGTGAAGGCTCGGCACCAGGTACAAGCACCGAACCT | GTSTEPSEGSAPGSPA | |
| | AGCGAGGGCAGCGCTCCCGGCAGCCCTGCCGGCAGCCCAACCTCA | GSPTSTEEGTSTEPSE | |
| | ACTGAGGAGGGCACCAGTACTGAGCCCAGCGAGGGATCAGCACCT | GSAPGTSTEPSEGSAP | |
| | GGCACCAGCACCGAACCTAGCGAGGGGAGCGCCCCTGGGACTAGC | GTSESATPESGPGTST | |
| | GAGTCAGCTACACCAGAGAGCGGGCCTGGAACTTCTACCGAACCC | EPSEGSAPGTSESATP | |
| | AGTGAGGGATCCGCTCCAGGCACCTCCGAATCCGCAACCCCCGAA | ESGPGSEPATSGSETP | |
| | TCCGGACCTGGCTCAGAGCCCGCCACCAGCGGGAGCGAAACCCCT | GTSTEPSEGSAPGTST | |
| | GGCACATCCACCGAGCCTAGCGAAGGGTCCGCACCCGGCACCAGT | EPSEGSAPGTSESATP | |
| | ACAGAGCCTAGCGAGGGATCAGCACCTGGCACCAGTGAATCTGCT | ESGPGTSESATPESGP | |
| | ACACCAGAGAGCGGCCCTGGAACCTCCGAGTCCGCTACCCCCGAG | GSPAGSPTSTEEGTSE | |
| | AGCGGGCCAGGTTCTCCTGCTGGCTCCCCCACCTCAACAGAAGAG | SATPESGPGSEPATSG | |
| | GGGACAAGCGAAAGCGCTACGCCTGAGAGTGGCCCTGGCTCTGAG | SETPGTSESATPESGP | |
| | CCAGCCACCTCCGGCTCTGAAACCCCTGGCACTAGTGAGTCTGCC | GTSTEPSEGSAPGTST | |
| | ACGCCTGAGTCCGGACCCGGGACCTCTACTGAGCCCTCGGAGGGG | EPSEGSAPGTSTEPSE | |
| | AGCGCTCCTGGCACGAGTACAGAACCTTCCGAAGGAAGTGCACCG | GSAPGTSTEPSEGSAP | |
| | GGCACAAGCACCGAGCCTTCCGAAGGCTCTGCTCCCGGAACCTCT | GTSTEPSEGSAPGTST | |
| | ACCGAACCCTCTGAAGGGTCTGCACCCGGCACGAGCACCGAACCC | EPSEGSAPGSPAGSPT | |
| | AGCGAAGGGTCAGCGCCTGGGACCTCAACAGAGCCCTCGGAAGGA | STEEGTSTEPSEGSAP | |
| | TCAGCGCCTGGAAGCCCTGCAGGGAGTCCAACTTCCACGGAAGAA | GTSESATPESGPGSEP | |
| | GGAACGTCTACAGAGCCATCAGAGGGGTCCGCACCAGGTACCAGC | ATSGSETPGTSESATP | |
| | GAATCCGCTACTCCCGAATCTGGCCCTGGGTCCGAACCTGCCACC | ESGPGSEPATSGSETP | |
| | TCCGGCTCTGAAACTCCAGGGACCTCCGAATCTGCCACACCCGAG | GTSESATPESGPGTST | |
| | AGCGGCCCTGGCTCCGAGCCCGCAACATCTGGCAGCGAGACACCT | EPSEGSAPGTSESATP | |
| | GGCACCTCCGAGAGCGCAACACCCGAGAGCGGCCCTGGCACCAGC | ESGPGSPAGSPTSTEE | |
| | ACCGAGCCATCCGAGGGATCCGCCCCAGGCACTTCTGAGTCAGCC | GSPAGSPTSTEEGSPA | |
| | ACACCCGAAAGCGGACCAGGATCACCCGCTGGCTCCCCCACCAGT | GSPTSTEEGTSESATP | |
| | ACCGAGGAGGGGTCCCCCGCTGGAAGTCCAACAAGCACTGAGGAA | ESGPGTSTEPSEGSAP | |
| | GGGTCCCCTGCCGGCTCCCCCACAAGTACCGAAGAGGGCACAAGT | GTSESATPESGPGSEP | |
| | GAGAGCGCCACTCCCGAGTCCGGGCCTGGCACCAGCACAGAGCCT | ATSGSETPGTSESATP | |
| | TCCGAGGGGTCCGCACCAGGTACCTCAGAGTCTGCTACCCCCGAG | ESGPGSEPATSGSETP | |
| | TCAGGGCCAGGATCAGAGCCAGCCACCTCCGGGTCTGAGACACCC | GTSESATPESGPGTST | |
| | GGGACTTCCGAGAGTGCCACCCCTGAGTCCGGACCCGGGTCCGAG | EPSEGSAPGSPAGSPT | |
| | CCCGCCACTTCCGGCTCCGAAACTCCCGGCACAAGCGAGAGCGCT | STEEGTSESATPESGP | |
| | ACCCCAGAGTCAGGACCAGGAACATCTACAGAGCCCTCTGAAGGC | GSEPATSGSETPGTSE | |
| | TCCGCTCCAGGGTCCCCAGCCGGCAGTCCCACTAGCACCGAGGAG | SATPESGPGSPAGSPT | |
| | GGAACCTCTGAAAGCGCCACACCCGAATCAGGGCCAGGGTCTGAG | STEEGSPAGSPTSTEE | |
| | CCTGCTACCAGCGGCAGCGAGACACCAGGCACCTCTGAGTCCGCC | GTSTEPSEGSAPGTSE | |
| | ACACCAGAGTCCGGACCCGGATCTCCCGCTGGGAGCCCCACCTCC | SATPESGPGTSESATP | |
| | ACTGAGGAGGGATCTCCTGCTGGCTCTCCAACATCTACTGAGGAA | ESGPGTSESATPESGP | |
| | GGTACCTCAACCGAGCCATCCGAGGGATCAGCTCCCGGCACCTCA | GSEPATSGSETPGSEP | |
| | GAGTCGGCAACCCCGGAGTCTGGACCCGGAACTTCCGAAAGTGCC | ATSGSETPGSPAGSPT | |
| | ACACCAGAGTCCGGTCCCGGGACTTCAGAATCAGCAACACCCGAG | STEEGTSTEPSEGSAP | |
| | TCCGGCCCTGGGTCTGAACCCGCCACAAGTGGTAGTGAGACACCA | GTSTEPSEGSAPGSEP | |
| | GGATCAGAACCTGCTACCTCAGGGTCAGAGACACCCGGATCTCCG | ATSGSETPGTSESATP | |
| | GCAGGCTCACCAACCTCCACTGAGGAGGGCACCAGCACAGAACCA | ESGPGTSTEPSEGSAP | |
| | AGCGAGGGCTCCGCACCCGGAACAAGCACTGAACCCAGTGAGGGT | EAGRSANHTPAGLTGP | |
| | TCAGCACCCGGCTCTGAGCCGGCCACAAGTGGCAGTGAGACACCC | GTAEAASASGMWELEK | |
| | GGCACTTCAGAGAGTGCCACCCCCGAGAGTGGCCCAGGCACTAGT | DVYVVEVDWTPDAPGE | |
| | ACCGAGCCCTCTGAAGGCAGTGCGCCAGAGGCCGGCCGGAGCGCC | TVNLTCDTPEEDDITW | |
| | AACCACACCCCCGCCGGCCTGACCGGCCCTGGCACAGCCGAGGCC | TSDQRHGVIGSGKTLT | |
| | GCTAGCGCCAGCGGCATGTGGGAGCTGGAGAAGGACGTGTACGTG | ITVKEFLDAGQYTCHK | |
| | GTGGAGGTGGACTGGACACCAGATGCCCCCGGCGAGACCGTGAAC | GGETLSHSHLLLHKKE | |
| | CTGACATGCGACACCCCCGAGGAGGACGATATCACCTGGACATCT | NGIWSTEILKNFKNKT | |
| | GATCAGAGGCACGGCGTGATCGGAAGCGGCAAGACCCTGACAATC | FLKCEAPNYSGRFTCS | |
| | ACCGTGAAGGAGTTCCTGGATGCCGGCCAGTACACATGTCACAAG | WLVQRNMDLKFNIKSS | |
| | GGCGGCGAGACCCTGTCCCACTCTCACCTGCTGCTGCACAAGAAG | SSSPDSRAVTCGMASL | |
| | GAGAACGGCATCTGGTCCACAGAGATCCTGAAGAACTTCAAGAAT | SAEKVTLDQRDYEKYS | |
| | AAGACCTTTCTGAAGTGCGAGGCCCCTAATTATAGCGGCCGGTTC | VSCQEDVTCPTAEETL | |
| | ACCTGTTCCTGGCTGGTGCAGAGAAACATGGACCTGAAGTTTAAT | PIELALEARQQNKYEN | |
| | ATCAAGAGCTCCTCTAGCTCCCCAGATAGCCGGGCAGTGACATGC | YSTSFFIRDIIKPDPP | |
| | GGAATGGCCAGCCTGTCCGCCGAGAAGGTGACCCTGGACCAGAGA | KNLQMKPLKNSQVEVS | |
| | GATTACGAGAAGTATTCTGTGAGCTGCCAGGAGGACGTGACATGT | WEYPDSWSTPHSYFSL | |
| | CCCACCGCCGAGGAGACACTGCCTATCGAGCTGGCCCTGGAGGCC | KFFVRIQRKKEKMKET | |
| | AGGCAGCAGAACAAGTACGAGAATTATTCCACCTCTTTCTTTATC | EEGCNQKGAFLVEKTS | |
| | CGCGACATCATCAAGCCAGATCCCCCTAAGAACCTGCAGATGAAG | TEVQCKGGNVCVQAQD | |
| | CCCCTGAAGAATTCCCAGGTCGAGGTGTCTTGGGAGTACCCTGAC | RYYNSSCSKWACVPCR | |
| | AGCTGGTCCACACCACACTCTTATTTCAGCCTGAAGTTCTTTGTG | VRSGGGGSGGGGSGGG | |
| | AGGATCCAGCGCAAGAAGGAGAAGATGAAGGAGACCGAGGAGGGC | GSRVIPVSGPARCLSQ | |
| | TGCAATCAGAAGGGCGCCTTTCTGGTGGAGAAGACATCCACCGAG | SRNLLKTTDDMVKTAR | |
| | GTGCAGTGCAAGGGAGGAAACGTGTGCGTGCAGGCACAGGATCGG | EKLKHYSCTAEDIDHE | |
| | TACTATAATTCTAGCTGTTCCAAGTGGGCCTGCGTGCCTTGTCGG | DITRDQTSTLKTCLPL | |
| | GTGAGATCTGGCGGCGGCGGCTCTGGCGGCGGCGGCTCCGGCGGC | ELHKNESCLATRETSS | |
| | GGCGGCTCCAGAGTGATCCCCGTGAGCGGACCAGCAAGGTGCCTG | TTRGSCLPPQKTSLMM | |
| | TCCCAGAGCCGGAACCTGCTGAAGACCACAGACGATATGGTGAAG | TLCLGSIYEDLKMYQT | |
| | ACCGCCCGGGAGAAGCTGAAGCACTACTCTTGTACAGCCGAGGAC | EFQAINAALQNHNHQQ | |
| | ATCGATCACGAGGACATCACCCGGGATCAGACCTCTACACTGAAG | IILDKGMLVAIDELMQ | |
| | ACATGCCTGCCCCTGGAGCTGCACAAGAACGAGAGCTGTCTGGCC | SLNHNGETLRQKPPVG | |
| | ACCCGGGAGACAAGCTCCACCACAAGAGGCAGCTGCCTGCCCCCT | EADPYRVKMKLCILLH | |
| | CAGAAGACCTCCCTGATGATGACCCTGTGCCTGGGCTCTATCTAC | AFSTRVVTINRVMGYL | |
| | GAGGACCTGAAGATGTATCAGACCGAGTTCCAGGCCATCAATGCC | SSA (SEQ ID NO: | |
| | GCCCTGCAGAACCACAATCACCAGCAGATCATCCTGGACAAGGGC | 865) | |
| | ATGCTGGTGGCCATCGATGAGCTGATGCAGAGCCTGAACCACAAT | | |
| | GGCGAGACCCTGAGGCAGAAGCCACCAGTGGGAGAGGCAGATCCT | | |
| | TACAGGGTGAAGATGAAGCTGTGCATCCTGCTGCACGCCTTTTCC | | |
| | ACCAGGGTGGTGACAATCAATCGCGTGATGGGCTATCTGTCTAGC | | |
| | GCC (SEQ ID NO: 847) | | |
| XP18 | GCAAGCTCCGCCACCCCAGAGTCCGGACCTGGCACCTCTACAGAG | ASSATPESGPGTSTEP | IL12 |
| | CCAAGCGAGGGATCCGCCCCAGGCACAAGCGAGTCCGCCACCCCA | SEGSAPGTSESATPES | |
| | GAGTCTGGACCAGGAAGCGGACCTGCCACCTCTGAGAGCGCCACA | GPGSGPATSESATPGT | |
| | CCAGGCACCTCCGAGTCTGCCACACCAGAGTCCGGACCAGGATCT | SESATPESGPGSEPAT | |
| | GAGCCTGCCACCAGCGGATCCGAGACACCTGGCACCTCTGAAAGC | SGSETPGTSESATPES | |
| | GCCACTCCAGAGAGCGGACCAGGCACCTCCACCGAGCCTTCTGAG | GPGTSTEPSEGSAPGS | |
| | GGAAGCGCCCCAGGAAGCCCTGCAGGATCCCCAACCTCTACAGAG | PAGSPTSTEEGTSESA | |
| | GAGGGCACATCCGAGTCTGCCACCCCTGAGAGCGGACCAGGATCC | TPESGPGSEPATSGSE | |
| | GAGCCAGCCACAAGCGGATCCGAGACACCAGGCACCTCTGAGAGC | TPGTSESATPESGPGS | |
| | GCCACGCCTGAATCCGGACCAGGAAGCCCAGCAGGAAGCCCCACC | PAGSPTSTEEGSPAGS | |
| | TCCACAGAGGAGGGATCCCCTGCAGGATCTCCAACCAGCACAGAG | PTSTEEGTSTEPSEGS | |
| | GAGGGCACCAGCACAGAGCCTTCCGAGGGCTCTGCCCCAGGCACA | APGTSESATPESGPGT | |
| | TCCGAATCTGCCACTCCTGAGTCTGGACCTGGCACAAGCGAATCC | SESATPESGPGTSESA | |
| | GCCACCCCCGAAAGCGGACCAGGCACATCTGAGAGCGCCACCCCT | TPESGPGSEPATSGSE | |
| | GAGTCTGGCCCAGGATCTGAGCCAGCCACATCCGGCTCTGAGACC | TPGSEPATSGSETPGS | |
| | CCTGGCAGCGAACCTGCCACAAGCGGCAGCGAGACCCCTGGAAGC | PAGSPTSTEEGTSTEP | |
| | CCAGCAGGCTCCCCCACCTCCACCGAAGAAGGCACCAGCACAGAG | SEGSAPGTSTEPSEGS | |
| | CCATCTGAGGGAAGCGCCCCTGGCACCAGCACCGAACCATCCGAG | APGSEPATSGSETPGT | |
| | GGATCTGCCCCAGGATCCGAGCCTGCCACCTCTGGCAGTGAAACC | SESATPEAGRSANHTP | |
| | CCTGGCACCTCCGAATCTGCCACACCCGAGGCAGGCCGGTCCGCC | AGLTGPGTSESATPES | |
| | AACCACACCCCAGCCGGCCTGACAGGACCTGGCACCAGCGAATCC | MWELEKDVYVVEVDWT | |
| | GCCACTCCAGAGAGCATGTGGGAGCTGGAGAAGGACGTGTACGTG | PDAPGETVNLTCDTPE | |
| | GTGGAGGTGGACTGGACACCCGATGCCCCTGGCGAGACCGTGAAT | EDDITWTSDQRHGVIG | |
| | CTGACATGCGACACCCCTGAGGAGGACGATATCACCTGGACATCC | SGKTLTITVKEFLDAG | |
| | GATCAGAGACACGGCGTGATCGGCTCTGGCAAGACCCTGACAATC | QYTCHKGGETLSHSHL | |
| | ACCGTGAAGGAGTTCCTGGATGCCGGCCAGTACACATGTCACAAG | LLHKKENGIWSTEILK | |
| | GGCGGCGAGACCCTGTCTCACAGCCACCTGCTGCTGCACAAGAAG | NFKNKTFLKCEAPNYS | |
| | GAGAACGGCATCTGGTCCACAGAGATCCTGAAGAACTTCAAGAAT | GRFTCSWLVQRNMDLK | |
| | AAGACCTTTCTGAAGTGCGAGGCCCCCAATTATAGCGGCAGGTTC | FNIKSSSSSPDSRAVT | |
| | ACCTGTTCCTGGCTGGTGCAGCGCAACATGGACCTGAAGTTTAAT | CGMASLSAEKVTLDQR | |
| | ATCAAGTCTAGCTCCTCTAGCCCTGATAGCAGGGCAGTGACATGC | DYEKYSVSCQEDVTCP | |
| | GGAATGGCATCCCTGTCTGCCGAGAAGGTGACCCTGGACCAGAGA | TAEETLPIELALEARQ | |
| | GATTACGAGAAGTATAGCGTGTCCTGCCAGGAGGACGTGACATGT | QNKYENYSTSFFIRDI | |
| | CCTACCGCCGAGGAGACCCTGCCAATCGAGCTGGCCCTGGAGGCC | IKPDPPKNLQMKPLKN | |
| | AGGCAGCAGAACAAGTACGAGAATTATTCTACCAGCTTCTTTATC | SQVEVSWEYPDSWSTP | |
| | CGCGACATCATCAAGCCAGATCCCCCTAAGAACCTGCAGATGAAG | HSYFSLKFFVRIQRKK | |
| | CCCCTGAAGAATTCCCAGGTGGAGGTGAGCTGGGAGTACCCAGAC | EKMKETEEGCNQKGAF | |
| | TCCTGGTCTACCCCCCACAGCTATTTCTCCCTGAAGTTCTTTGTG | LVEKTSTEVQCKGGNV | |
| | AGGATCCAGCGCAAGAAGGAGAAGATGAAGGAGACCGAGGAGGGC | CVQAQDRYYNSSCSKW | |
| | TGCAACCAGAAGGGCGCCTTTCTGGTGGAGAAGACATCCACCGAG | ACVPCRVRSGTATPES | |
| | GTGCAGTGCAAGGGAGGAAACGTGTGCGTGCAGGCACAGGATAGG | GPGEAGRSANHTPAGL | |
| | TACTATAATTCCTCTTGTAGCAAGTGGGCATGCGTGCCATGTCGG | TGPATPESGPGSPAGS | |
| | GTGAGATCCGGCACAGCTACTCCTGAATCTGGACCAGGAGAGGCA | PTSTEEGSPAGSPTST | |
| | GGCCGCAGCGCCAACCACACCCCTGCAGGACTGACAGGACCAGCA | EEGSPAGSPTSTEEGT | |
| | ACCCCAGAGAGCGGACCTGGATCCCCAGCCGGCTCTCCAACAAGC | SESATPESGPGTSTEP | |
| | ACCGAAGAAGGATCTCCAGCAGGATCCCCAACATCTACCGAGGAG | SEGSAPGTSESATPES | |
| | GGCTCCCCAGCAGGAAGCCCTACATCCACCGAGGAGGGCACAAGC | GPGSEPATSGSETPGT | |
| | GAGTCCGCCACGCCAGAGTCCGGACCAGGCACATCTACCGAACCA | SESATPESGPGSEPAT | |
| | AGCGAAGGAAGCGCCCCTGGCACATCTGAAAGCGCCACTCCCGAA | SGSETPGTSESATPES | |
| | AGCGGACCAGGAAGCGAGCCAGCCACCTCCGGATCTGAGACACCA | GPGTSTEPSEGSAPGS | |
| | GGCACCAGCGAGTCCGCCACACCTGAGTCTGGGCCTGGCTCTGAG | PAGSPTSTEEGTSESA | |
| | CCAGCCACCTCTGGAAGTGAAACCCCCGGCACCTCCGAGTCTGCC | TPESGPGSEPATSGSE | |
| | ACGCCTGAGAGCGGACCAGGCACATCCACCGAGCCTAGCGAAGGC | TPGTSESATPESGPGS | |
| | TCTGCCCCTGGCAGCCCTGCCGGCTCCCCTACATCCACTGAGGAG | PAGSPTSTEEGSPAGS | |
| | GGCACAAGCGAGTCCGCCACTCCTGAAAGCGGACCTGGATCCGAA | PTSTEEGTSTEPSEGS | |
| | CCTGCCACCTCTGGAAGTGAGACCCCTGGCACCTCCGAGTCTGCC | APGTSESATPESGPGT | |
| | ACCCCCGAATCTGGCCCTGGCTCCCCAGCAGGCTCTCCCACAAGC | SESATPESGPGTSPSA | |
| | ACCGAGGAGGGATCCCCAGCAGGATCCCCTACATCTACTGAAGAG | TPESGPGSEPATSGSE | |
| | GGCACAAGCACCGAACCTAGCGAGGGATCCGCCCCTGGCACAAGC | TPGSEPATSGSETPGS | |
| | GAGTCCGCCACACCCGAATCTGGCCCCGGCACATCTGAAAGCGCC | PAGSPTSTEEGTSTEP | |
| | ACGCCAGAATCCGGCCCAGGCACATCCCCATCTGCCACCCCTGAG | SEGSAPGTSTEPSEGS | |
| | TCTGGGCCTGGGTCTGAACCTGCCACAAGCGGGAGCGAGACCCCT | APGSEPATSGSETPGT | |
| | GGCAGCGAGCCAGCCACATCTGGATCCGAAACTCCAGGCTCCCCA | SESAGASSATPESGPG | |
| | GCAGGATCCCCCACAAGCACTGAAGAAGGCACAAGCACCGAGCCT | TSTEPSEGSAPGTSES | |
| | AGCGAGGGGTCTGCCCCTGGCACATCTACCGAGCCCTCCGAAGGC | ATPESGPGSGPGTSES | |
| | TCCGCCCCAGGAAGCGAGCCTGCCACCTCCGGCTCTGAGACACCT | ATPGTSESATPESGPG | |
| | GGCACCAGCGAGTCCGCCGGAGCCTCCTCCGCCACTCCTGAATCC | SEPATSGSETPGTSES | |
| | GGACCTGGCACAAGCACTGAACCTTCCGAAGGAAGCGCCCCCGGC | ATPESGPGTSTEPSEG | |
| | ACATCTGAGAGCGCCACTCCAGAATCCGGACCAGGATCCGGCCCC | SAPGSPAGSPTSTEEG | |
| | GGCACCTCCGAGTCTGCCACTCCCGGCACCAGCGAATCCGCCACG | TSESATPESGPGSEPA | |
| | CCTGAGTCCGGCCCTGGGAGCGAACCCGCCACCTCTGGAAGCGAA | TSGSETPGTSESATPE | |
| | ACCCCAGGCACCTCCGAATCTGCCACGCCTGAGTCTGGCCCAGGC | SGPGSPAGSPTSTEEG | |
| | ACATCTACTGAACCTAGCGAAGGGTCTGCCCCTGGGAGCCCTGCA | SPAGSPTSTEEGTSTE | |
| | GGCAGCCCCACATCCACAGAAGAAGGCACAAGCGAATCCGCCACA | PSEGSAPGTSESATPE | |
| | CCTGAGTCCGGACCTGGATCCGAGCCCGCCACCTCTGGCTCCGAA | SGPGTSESATPESGPG | |
| | ACTCCTGGCACCTCCGAGTCTGCCACGCCGGAATCTGGACCAGGA | TSESATPESGPGSEPA | |
| | TCTCCTGCCGGATCCCCCACAAGCACAGAAGAAGGGAGCCCTGCC | TSGSETPGSEPATSGS | |
| | GGATCCCCTACATCTACAGAAGAGGGCACAAGCACTGAGCCCTCC | ETPGSPAGSPTSTEEG | |
| | GAAGGGTCCGCCCCCGGCACAAGCGAGTCCGCCACGCCGGAAAGT | TSTEPSEGSAPGTSTE | |
| | GGCCCTGGCACATCTGAGAGCGCCACACCCGAGTCTGGGCCAGGC | PSEGSAPGSEPATSGS | |
| | ACATCCGAGTCTGCCACGCCAGAGTCTGGACCTGGAAGTGAACCC | ETPGTSESATPEAGRS | |
| | GCCACAAGCGGCTCCGAGACTCCTGGCAGCGAGCCTGCCACATCT | ANHTPAGLTGPGTSES | |
| | GGATCCGAGACTCCTGGAAGCCCAGCAGGATCACCCACAAGCACT | ATPESRVIPVSGPARC | |
| | GAGGAGGGCACATCCACCGAGCCCAGCGAGGGATCTGCCCCTGGC | LSQSRNLLKTTDDMVK | |
| | ACATCCACAGAACCTTCCGAAGGATCCGCCCCTGGCTCCGAACCT | TAREKLKHYSCTAEDI | |
| | GCCACCTCCGGGAGCGAAACCCCAGGCACCAGCGAATCCGCCACC | DHEDITRDQTSTLKTC | |
| | CCAGAGGCAGGCCGGAGCGCCAACCACACCCCCGCTGGACTGACC | LPLELHKNESCLATRE | |
| | GGCCCTGGCACCTCTGAGAGCGCCACCCCAGAGTCTAGAGTGATC | TSSTTRGSCLPPQKTS | |
| | CCTGTGAGCGGACCAGCAAGGTGCCTGTCCCAGTCTAGAAATCTG | LMMTLCLGSIYEDLKM | |
| | CTGAAGACCACAGACGATATGGTGAAGACAGCCAGGGAGAAGCTG | YQTEFQAINAALQNHN | |
| | AAGCACTACAGCTGTACCGCCGAGGACATCGATCACGAGGACATC | HQQIILDKGMLVAIDE | |
| | ACACGCGATCAGACATCCACCCTGAAGACCTGCCTGCCCCTGGAG | LMQSLNHNGETLRQKP | |
| | CTGCACAAGAACGAGAGCTGTCTGGCCACACGGGAGACCTCTAGC | PVGEADPYRVKMKLCI | |
| | ACCACAAGAGGCAGCTGCCTGCCACCCCAGAAGACATCCCTGATG | LLHAFSTRVVTINRVM | |
| | ATGACCCTGTGCCTGGGCAGCATCTACGAGGACCTGAAGATGTAT | GYLSSAGTATPESGPG | |
| | CAGACCGAGTTCCAGGCCATCAATGCCGCCCTGCAGAACCACAAT | EAGRSANHTPAGLTGP | |
| | CACCAGCAGATCATCCTGGACAAGGGCATGCTGGTGGCCATCGAT | ATPESGPGSEPATSGS | |
| | GAGCTGATGCAGTCCCTGAACCACAATGGCGAGACCCTGAGGCAG | ETPGTSESATPESGPG | |
| | AAGCCTCCAGTGGGAGAGGCCGATCCCTACAGAGTGAAGATGAAG | SPAGSPTSTEEGSPAG | |
| | CTGTGCATCCTGCTGCACGCCTTTAGCACAAGGGTGGTGACCATC | SPTSTEEGTSTEPSEG | |
| | AACCGCGTGATGGGCTATCTGTCCTCTGCCGGAACAGCAACCCCT | SAPGTSESATPESGPG | |
| | GAATCTGGACCTGGAGAGGCAGGCAGGAGCGCCAATCACACCCCA | TSESATPESGPGTSAS | |
| | GCCGGGCTGACCGGCCCAGCAACCCCTGAGTCCGGCCCAGGGTCC | ATPESGPGSEPATSGS | |
| | GAGCCAGCCACCAGCGGCAGCGAAACTCCAGGCACCTCTGAGAGC | ETPGSEPATSGSETPG | |
| | GCCACTCCTGAGTCCGGGCCAGGATCCCCAGCAGGATCTCCTACA | SPAGSPTSTEEGTSTE | |
| | AGCACTGAAGAAGGGTCTCCCGCCGGCAGCCCAACATCTACTGAG | PSEGSAPGTSTEPSEG | |
| | GAAGGCACAAGCACTGAACCCTCCGAAGGATCCGCCCCCGGCACA | SAPGSEPATSGSETPG | |
| | TCCGAGTCTGCCACTCCTGAGAGCGGACCCGGCACAAGCGAGTCC | TSESAGEPEA (SEQ | |
| | GCCACGCCTGAAAGTGGACCAGGCACATCTGCCAGCGCCACTCCA | ID NO: 866) | |
| | GAAAGCGGCCCTGGAAGCGAACCTGCCACATCCGGCTCCGAGACC | | |
| | CCCGGCTCTGAACCAGCCACAAGCGGCAGCGAAACTCCCGGAAGC | | |
| | CCAGCAGGATCTCCCACAAGCACTGAAGAGGGCACAAGCACGGAG | | |
| | CCTAGCGAAGGATCTGCCCCCGGCACAAGCACTGAACCCAGTGAA | | |
| | GGATCCGCCCCAGGCAGCGAACCAGCCACCTCTGGAAGCGAGACC | | |
| | CCTGGCACCTCCGAGTCTGCCGGAGAGCCTGAGGCCTGA (SEQ | | |
| | ID NO: 848) | | |

### Example 11: In Vivo Effects of IL12-XPAC-4X Test Compound on Mouse Model

Toxicity of IL-12-XPAC-4X was monitored in C27/Blk6 mouse model bearing MC38 tumors. This murine model was used to compare the toxicity effects of the test compound with muIL2. The test article was administered every 3 days in non-tumor bearing mice (D03, D13, D16, and D19). No significant toxicity (as measured by body weight loss) was seen in this model at the doses administered. These data are shown in FIG. 151B, which shows that in these non-tumor bearing mice, there was no sign of toxicity in the mice treated with XPAC as measured by changes in body weight. In the mice treated with IL12, however, there was a dose-dependent toxicity as evidenced by a percentage loss of body weight.

The following Table shows the in vivo study design for testing rIL-12 and IL-12 XPAC efficacy:

| | | | | Daily | Molar | |
|---|---|---|---|---|---|---|
| Group | Group | | | Molar | Dose over | No. |
| No. | Identity | Dose | Frequency | Dose | 4 Days | of Mice |
| 1 | Control | Diluent | Every 4 | 0 | 0 | 8 |
| | | | day | | | |
| 2 | muIL-12 | 30 ug | Daily | 516 pmol | 2064 pmol | 5 |
| 3 | muIL-12 | 50 ug | Daily | 860 pmol | 3440 pmol | 5 |
| 4 | muIL-12 | 50 ug | Every 4 | 860 pmol | 860 pmol | 5 |
| | | | Days | | | |
| 5 | IL-12- | 300 ug | Every 4 | 1720 pmol | 1720 pmol | 5 |
| | XPAC | | Days | | | |
| 6 | IL-12- | 450 ug | Every 4 | 2580 pmol | 2580 pmol | 5 |
| | XPAC | | Days | | | |

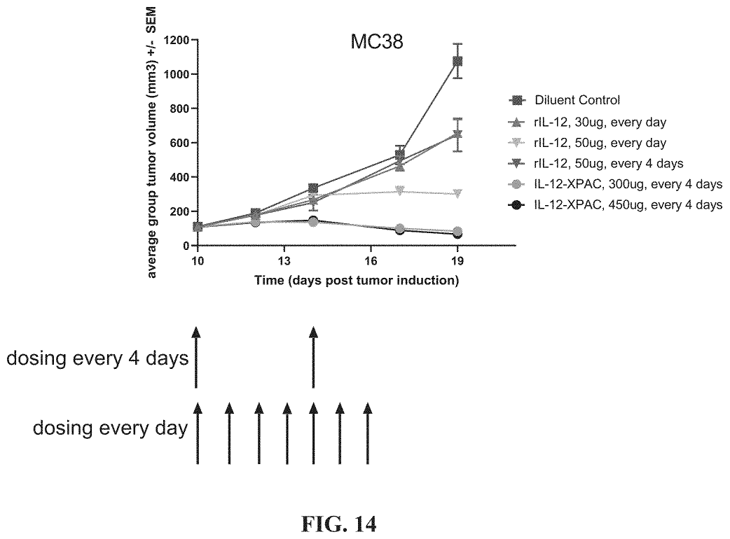

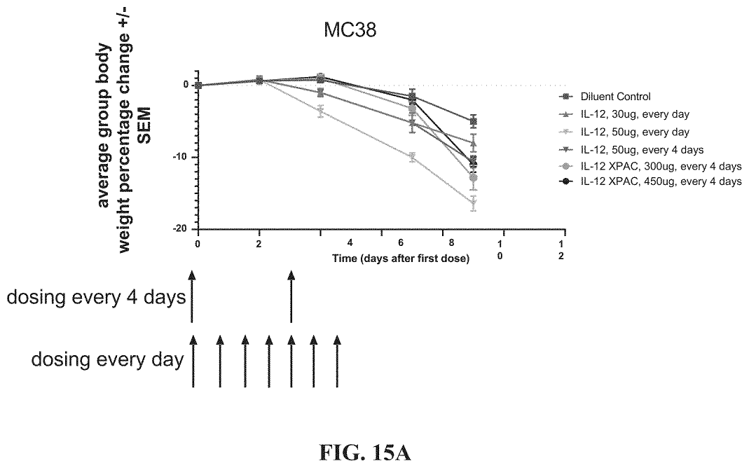

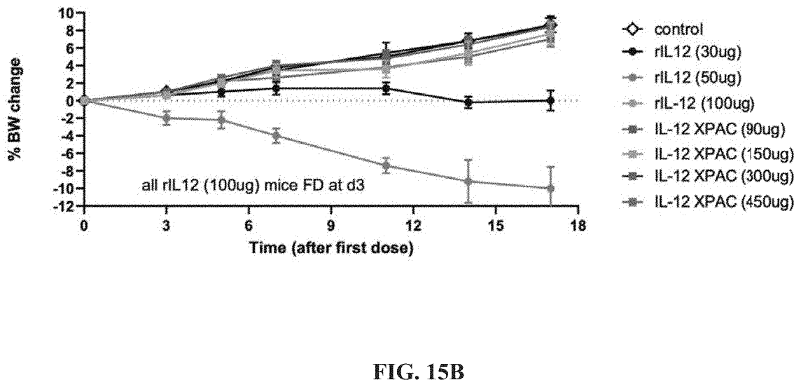

FIG. 14 shows the tumor regression data generated from the above-outlined study. There was a significant decrease in tumor volume in the mice treated with IL-12 XPAC (Groups 5 and 6) as compared to mice in the Control group (Group 1). Comparatively, there was little to no tumor regression in mice treated with rIL-12 (Groups 2, 3, and 4). FIG. 15A shows the toxicity/body weight data for the aforementioned groups and showed there were no changes in body weight as a result of administration of the test article.

### Example 12: Xtenylated IL12 Constructs Comprising a Tumor Targeting Domain

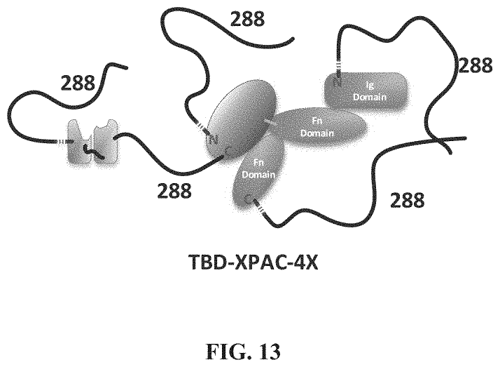

FIG. 13 shows an additional exemplary embodiment of the present disclosure in which an XPAC further comprises a tumor targeting domain. While this figure shows the tumor targeting domain on one chain, it should be understood that the tumor targeting domain may be present on more than one chain and may be present on one of the other XTEN chains. The position of the tumor targeting domain should be such that it does not interfere with the masking of the cytokine and also such that it is able to recognize the antigen against which the tumor targeting domain is targeted.

The tumor targeting domain may in exemplary embodiments also be Xtenylated. Ideally, the tumor targeting domain is one that is expressed on tumor cells but is absent in healthy tissue. For example, in tumors and in chronic inflammatory conditions, tissue remodeling and neovascularization processes expose antigens, which are otherwise virtually undetectable in healthy organs. One example is represented by splice variants of fibronectin, a glycoprotein a glycoprotein of the extracellular matrix (ECM). The extra-domains A and B (EDA and EDB) of fibronectin are strongly expressed in tumors, at sites of tissue remodeling and during fetal development, but are otherwise not found in normal tissues, exception made for the female reproductive system. Similarly, splice variants of tenascin-C are specifically found in tissues and tumors undergoing neo-angiogenesis, in a process which is regulated by intracellular pH. Therefore, EDA, EDB and splice variants of tenascin-C represent suitable targets for the delivery of bioactive payloads like cytokines. In oncological malignancies molecular targets may include fibroblast activation protein (FAP), cellular antigens (e.g., CEA and PSMA) or proteins, which become accessible in necrotic lesions, such as histones. Antibodies which have been extensively characterized in the context of cytokine fusions include F8 (targeting EDA-fibronectin; See US Publication 20210163579 for exemplary EDA targeting antibodies), L19 (targeting EDB-fibronectin; US Publication 20200397915), F16 (targeting the A1 domain of tenascin-C), scFv36 (targeting FAP), hu14.18 (targeting the GD2 ganglioside), chCLL-1 (targeting CD20) and anti-HER2/neu.

Simply by way of example, those of skill in the art are referred to US Publication 20200397915 which provides a detailed description of IL-12 constructs designed to target fibronectin EDB. US Publication 20210163579 shows exemplary constructs that target ED-A of fibronectin. The ED-A of fibronectin has been shown to be a marker of tumor angiogenesis, and the F8 antibody has been used for tumor targeting alone (WO2008/12001, WO2009/0136619, WO2011/015333) or fused to TNF or IL2 or both (Villa et al. (2008) Int. J. Cancer 122, 2405-2413; Hemmerle et al. (2013) Br. J. Cancer 109, 1206-1213; Frey et al. (2008) J. Urol. 184, 2540-2548, WO2010/078945, WO2008/120101, WO2016/180715), to IL4 (WO2014/173570), orto IL12 (WO2013/014149).

A particularly preferred tumor targeting domain for use in the XPACs of the invention is the L19 antibody or functional variants thereof described in US Publication 20200397915. The following Table 23 shows the sequences of the variable heavy and light chains of L19 as well as the CDR sequences from those chains.

TABLE 23
Exemplary L19 Antibody Sequences for Use as Tumor Binding Domain
in XPACs

| | |
|---|---|
| L19 VH | EVQLLESGGGLVQPGGSLRLSCAASGFTFSSFSMSWVRQAPGKGLEWVSS |
| | ISGSSGTTYYADSVKGRFTISRDNSKNTLYLQMNSLRAEDTAVYYCAKPF |
| | PYFDYWGQGTLVTVSS (SEQ ID NO: 159) |
| L19 VL | EIVLTQSPGTLSLSPGERATLSCRASQSVSSSFLAWYQQKPGQAPRLLIY |
| | YASSRATGIPDRFSGSGSGTDFTLTISRLEPEDFAVYYCQQTGRIPPTFG |
| | QGTKVEIK (SEQ ID NO: 160) |
| L19 CDR1 VH | SFSMS (SEQ ID NO: 161) |
| L19 CDR2 VH | SISGSSGTTYYADSVKG (SEQ ID NO: 162) |
| L19 CDR3 VH | PFPYFDY (SEQ ID NO: 163) |
| L19 CDR1 VL | RASQSVSSSFLA (SEQ ID NO: 164) |
| L19 CDR2 VL | YASSRAT (SEQ ID NO: 165) |
| L19 CDR3 VL | QQTGRIPPT (SEQ ID NO: 166) |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A fusion protein comprising:
(a) an extended recombinant polypeptide characterized in that:
i) it comprises at least 12 amino acid residues;
ii) at least 90% of the amino acid residues of the extended recombinant polypeptide are selected from glycine (G), alanine (A), serine (S), threonine (T), glutamate (E) and proline (P); and
iii) it has 4-6 different amino acid residues selected from G, A, S, T, E and P;
(b) a cytokine; and
(c) a linker joining the cytokine and the extended recombinant polypeptide, wherein the linker comprises a release segment (RS).

2. The fusion protein of claim 1, wherein said fusion protein comprises 1, 2, 3, 4 or more extended recombinant polypeptides.

3. The fusion protein of claim 1, wherein said fusion protein further comprises a tumor targeting domain.

4. The fusion protein of claim 1, wherein said RS is capable of being cleaved by at least one mammalian protease.

5-7. (canceled)

8. The fusion protein of claim 1, wherein the fusion protein has a structural arrangement, from N- to C-terminus of XTEN-RS-cytokine or cytokine-RS-XTEN.

9. The fusion protein of claim 1, wherein the cytokine is selected from a group consisting of interleukins, chemokines, interferons, tumor necrosis factors, colony-stimulating factors, or TGF-Beta superfamily members.

10-11. (canceled)

12. The fusion protein of claim 9, wherein the cytokine is IL-12 or an IL-12 variant.

13. The fusion protein of claim 1, wherein the cytokine comprises a first cytokine fragment (Cy1) and a second cytokine fragment (Cy2).

14. The fusion protein of claim 13, wherein Cy1 comprises an IL-12 p35 subunit.

15. The fusion protein of claim 14, wherein Cy2 comprises an IL-12 p40 subunit sequence identity to an interleukin-12 subunit alpha.

16-17. (canceled)

18. The fusion protein of claim 13, wherein the cytokine comprises a linker positioned between the first cytokine fragment (Cy1) and the second cytokine fragment (Cy2).

19. The fusion protein of claim 18 wherein said fusion protein comprises a Cy1 fragment that comprises an extended recombinant polypeptide at the N terminus and an extended recombinant polypeptide at the C-terminus.

20. The fusion protein of claim 18 wherein said fusion protein comprises a Cy2 fragment that comprises an extended recombinant polypeptide at the N terminus and an extended recombinant polypeptide at the C-terminus.

21. (canceled)

22. The fusion protein of claim 1, wherein the extended recombinant polypeptide sequence consists of multiple non-overlapping sequence motifs, wherein the sequence motifs are selected from the sequence motifs of Table 1.

23-27. (canceled)

28. A pharmaceutical composition, comprising the fusion protein of claim 1 and at least one pharmaceutically acceptable carrier.

29-30. (canceled)

31. A method of treating or preventing a disease or condition in a subject, the method comprising administering to a subject a therapeutically effective amount of the fusion protein of claim 1.

32-34. (canceled)

35. A fusion protein comprising a disulfide-linked heterodimer,
wherein the disulfide-linked heterodimer comprises a first subunit and a second subunit,
wherein the first subunit comprises the following elements in a N-to-C terminal or a C-to-N terminal orientation:
(a) an extended recombinant polypeptide characterized in that:
i) it comprises at least 12 amino acid residues;
ii) at least 90% of the amino acid residues of the extended recombinant polypeptide are selected from glycine (G), alanine (A), serine (S), threonine (T), glutamate (E) and proline (P); and
iii) it has 4-6 different amino acid residues selected from G, A, S, T, E and P;
(b) a release segment (RS); and
(c) a first cytokine fragment (Cy1);
wherein the second subunit comprises the following elements in a N-to-C terminal or a C-to-N terminal orientation:
(d) an extended recombinant polypeptide characterized in that:
i) it comprises at least 12 amino acid residues;
ii) at least 90% of the amino acid residues of the extended recombinant polypeptide are selected from glycine (G), alanine (A), serine (S), threonine (T), glutamate (E) and proline (P); and
iii) it has 4-6 different amino acid residues selected from G, A, S, T, E and P;
(e) a release segment (RS); and
(f) a second cytokine fragment (Cy2).

36. The fusion protein of claim 35, wherein Cy1 is an IL-12 p35 subunit.

37. The fusion protein of claim 35, wherein Cy2 is an IL-12 p40 subunit.

38. A kit comprising at least a first container, the first container comprising:
(a) an amount of a fusion protein sufficient to treat a disease, condition, or disorder upon administration to a subject in need thereof,
(b) an amount of a pharmaceutically acceptable carrier, together in a formulation ready for injection or for reconstitution with sterile water, buffer, or dextrose.

39. The kit of claim 35, wherein the kit further comprises:
(a) a label identifying the fusion protein, storage and handling conditions,
(b) a sheet of the approved indications for the fusion protein,
(c) instructions for the reconstitution and/or administration of the fusion protein for the use for the prevention and/or treatment of an approved indication,
(d) appropriate dosage and safety information,
(e) information identifying the lot and expiration of the drug, or
(f) any combination of (a)-(e).
